(19) United States
(12) Patent Application Publication
(10) Pub. No.: US 2022/0115598 A1
(43) Pub. Date: Apr. 14, 2022

(54) LUMINESCENCE DEVICE AND AMINE COMPOUND FOR LUMINESCENCE DEVICE

(71) Applicant: Samsung Display Co., Ltd., Yongin-si (KR)

(72) Inventor: Takuya UNO, Yokohama (JP)

(73) Assignee: Samsung Display Co., Ltd., Yongin-si (KR)

(21) Appl. No.: 17/365,486

(22) Filed: Jul. 1, 2021

(30) Foreign Application Priority Data
Oct. 12, 2020 (KR) 10-2020-0130927

(51) Int. Cl. H01L 51/00

(52) U.S. Cl. CPC H01L 51/0061; H01L 51/0073; H01L 51/5056; H01L 51/006; H01L 51/0058; H01L 51/0074

(57) ABSTRACT

A luminescence device of an embodiment includes a first electrode, a hole transport region disposed on the first electrode, an emission layer disposed on the hole transport region, an electron transport region disposed on the emission layer, and a second electrode disposed on the electron transport region. The hole transport region includes an amine compound represented by Formula 1, thereby providing high emission efficiency.

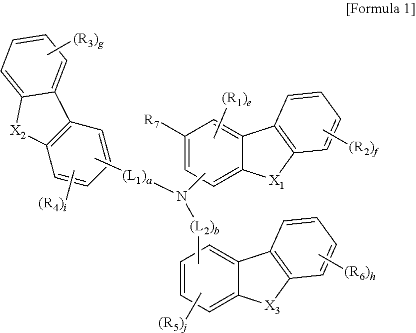

### CROSS-REFERENCE TO RELATED APPLICATION(S)

This application claims priority to and benefits of Korean Patent Application No. 10-2020-0130927 under 35 U.S.C. § 119, filed on Oct. 12, 2020 in the Korean Intellectual Property Office, the entire contents of which are incorporated herein by reference.

### BACKGROUND

### 1. Technical Field

The disclosure relates to a luminescence device and an amine compound for a luminescence device.

### 2. Description of the Related Art

Active development continues for an organic electroluminescence display as an image display. The organic electroluminescence display is different from a liquid crystal display and is a so-called self-luminescent display in which holes and electrons respectively injected from a first electrode and a second electrode recombine in an emission layer so that a light-emitting material including an organic compound in the emission layer emits light to achieve display.

In the application of an organic electroluminescence device to a display, there is a need to increase the emission efficiency and life of an organic electroluminescence device, and continuous development is required for materials for an organic electroluminescence device which stably achieves such characteristics.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

The disclosure provides a luminescence device with high efficiency and an amine compound included in a hole transport region of a luminescence device.

An embodiment provides an amine compound represented by Formula 1 below.

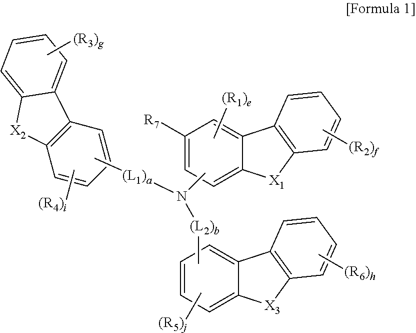

In Formula 1, X₁, X₂, and X₃ may each independently be O or S, and R₁ to R₆ may each independently be a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, or may be combined with an adjacent group to form a ring. In Formula 1, R₇ may be a hydrogen atom, a deuterium atom, a halogen atom, or a substituted or unsubstituted alkyl group of 1 to 20 carbon atoms, and L₁ and L₂ may each independently be a substituted or unsubstituted arylene group of 6 to 30 ring-forming carbon atoms, where a heteroaryl group is excluded. In Formula 1, a and b may each independently be an integer from 1 to 3, e may be an integer from 0 to 2, f to h may each independently be an integer from 0 to 4, i and j may each independently be an integer from 0 to 3, and X₁, X₂, and X₃ may not be O at the same time.

In an embodiment, Formula 1 may be represented by Formula 2 below.

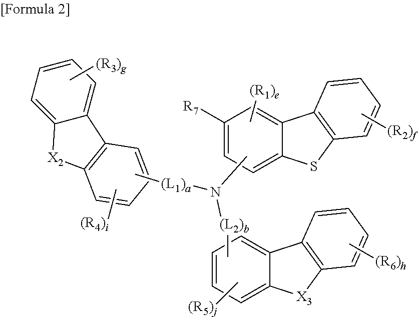

In Formula 2, X₂, X₃, R₁ to R₇, L₁, L₂, a, b, and e to j may be the same as defined in connection with Formula 1.

In an embodiment, Formula 1 may be represented by Formula 3 below.

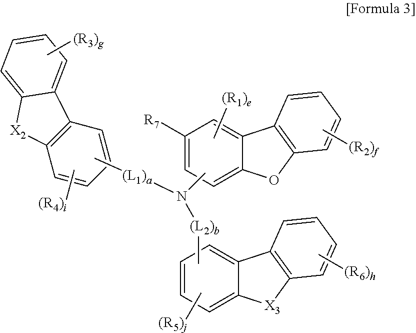

In Formula 3, X₂, X₃, R₁ to R₇, L₁, L₂, a, b, and e to j may be the same as defined in connection with Formula 1.

In an embodiment, Formula 2 may be represented by any one among Formula 4-1 to Formula 4-3 below.

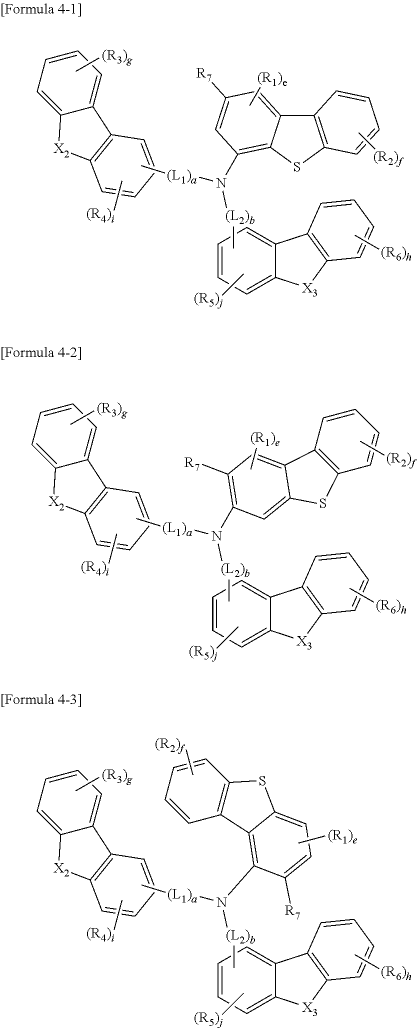

In Formula 4-1 to Formula 4-3, X₂, X₃, R₁ to R₇, L₁, L₂, a, b, and e to j may be the same as defined in connection with Formula 2.

In an embodiment, Formula 3 may be represented by any one among Formula 5-1 to Formula 5-3 below.

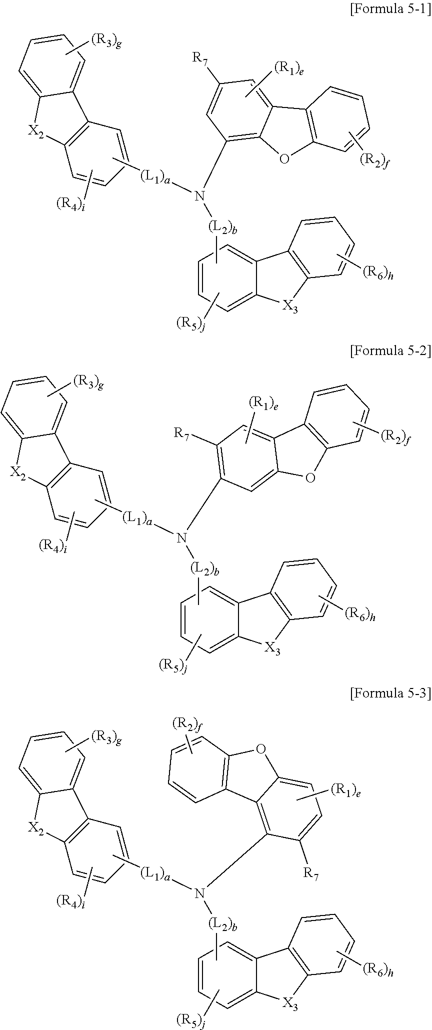

In Formula 5-1 to Formula 5-3, X₂, X₃, R₁ to R₇, L₁, L₂, a, b, and e to j may be the same as defined in connection with Formula 3.

In an embodiment, a and b may each be 1, and L₁ and L₂ may each independently be a substituted or unsubstituted phenylene group, a substituted or unsubstituted biphenylene group, a substituted or unsubstituted terphenyl group, a substituted or unsubstituted naphthylene group, or a substituted or unsubstituted phenanthrylene group.

In an embodiment, L₁ and L₂ may each independently be represented by any one among L-1 to L-11 below.

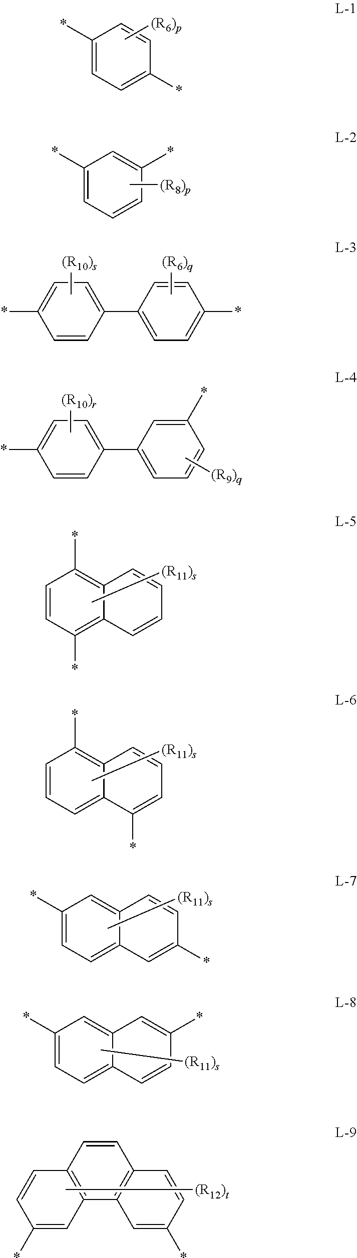

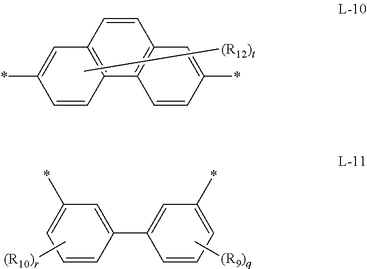

In L-1 to L-11, R₈ to R₁₂ may each independently be a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, p to r may each independently be an integer from 0 to 4, s may be an integer from 0 to 6, t may be an integer from 0 to 8, and * indicates a binding site to a neighboring atom.

In an embodiment, the amine compound represented by Formula 1 may be at least one selected among the compounds represented in Compound Group 1.

In an embodiment, the amine compound represented by Formula 1 may be at least one selected among the compounds represented in Compound Group 2.

An embodiment provides a luminescence device which may include a first electrode, a hole transport region disposed on the first electrode, an emission layer disposed on the hole transport region, an electron transport region disposed on the emission layer, and a second electrode disposed on the electron transport region, wherein the hole transport region may include an amine compound according to an embodiment.

In an embodiment, the hole transport region may include a hole injection layer disposed on the first electrode, and a hole transport layer disposed on the hole injection layer, wherein the hole transport layer may include an amine compound according to an embodiment.

In an embodiment, the hole transport region may include a hole transport layer disposed on the first electrode, and an electron blocking layer disposed on the hole transport layer, wherein the electron blocking layer may include an amine compound according to an embodiment.

### BRIEF DESCRIPTION OF THE DRAWINGS

The accompanying drawings are included to provide a further understanding of the embodiments, and are incorporated in and constitute a part of this specification. The drawings illustrate embodiments of the disclosure. The above and other aspects and features of the disclosure will become more apparent by describing in detail embodiments thereof with reference to the attached drawings, in which:

FIG. 1 is a plan view showing a display apparatus according to an embodiment;

FIG. 2 is a schematic cross-sectional view showing a display apparatus according to an embodiment;

FIG. 3 is a schematic cross-sectional view showing a luminescence device according to an embodiment;

FIG. 4 is a schematic cross-sectional view showing a luminescence device according to an embodiment;

FIG. 5 is a schematic cross-sectional view showing a luminescence device according to an embodiment;

FIG. 6 is a schematic cross-sectional view showing a luminescence device according to an embodiment;

FIG. 7 is a schematic cross-sectional view showing a display apparatus according to an embodiment; and

FIG. 8 is a schematic cross-sectional view showing a display apparatus according to an embodiment.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like numbers refer to like elements throughout.

In the description, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the description, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

The term “at least one of” is intended to include the meaning of “at least one selected from” for the purpose of its meaning and interpretation. For example, “at least one of A and B” may be understood to mean “A, B, or A and B.” When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the invention. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, 10%, or 5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

Hereinafter, embodiments will be explained with reference to attached drawings.

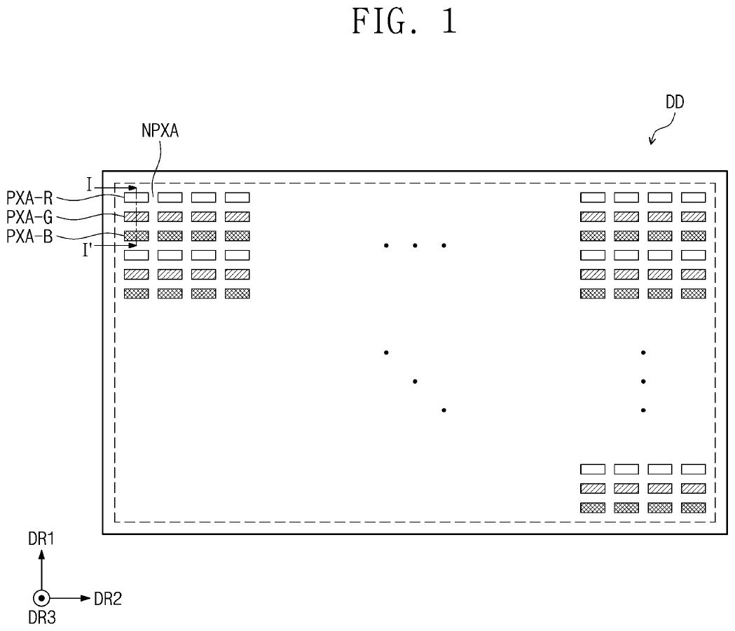

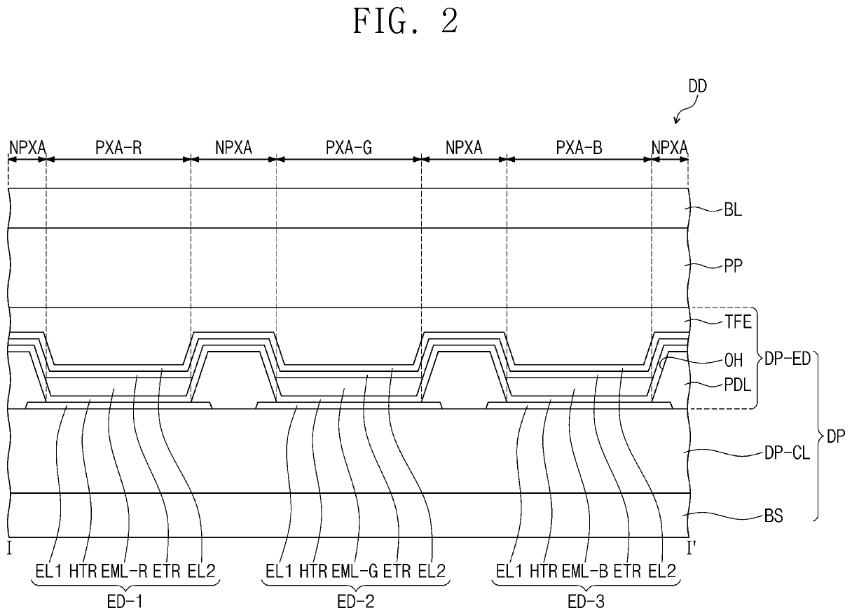

FIG. 1 is a plan view showing an embodiment of a display apparatus DD. FIG. 2 is a schematic cross-sectional view showing a display apparatus DD of an embodiment. FIG. 2 is a schematic cross-sectional view showing a part corresponding to line I-I′ in FIG. 1.

A display apparatus DD may include a display panel DP and an optical layer PP disposed on the display panel DP. The display panel DP includes luminescence devices ED-1, ED-2, and ED-3. The display apparatus DD may include multiple luminescence devices ED-1, ED-2, and ED-3. The optical layer PP may be disposed on the display panel DP and control light reflected from an external light at the display panel DP. The optical layer PP may include, for example, a polarization layer or a color filter layer. While not shown in the drawings, the optical layer PP may be omitted in the display apparatus DD in another embodiment.

The display panel DP may include a base layer BS, a circuit layer DP-CL provided on the base layer BS and a display device layer DP-ED. The display device layer DP-ED may include a pixel definition layer PDL, luminescence devices ED-1, ED-2, and ED-3 disposed between the pixel definition layers PDL, and an encapsulating layer TFE disposed on the luminescence devices ED-1, ED-2, and ED-3.

The base layer BS may be a member providing a base surface where the display device layer DP-ED is disposed. The base layer BS may be a glass substrate, a metal substrate, a plastic substrate, etc. However, embodiments are not limited thereto, and the base layer BS may be an inorganic layer, an organic layer, or a composite material layer.

In an embodiment, the circuit layer DP-CL may be disposed on the base layer BS, and the circuit layer DP-CL may include multiple transistors (not shown). Each of the transistors (not shown) may include a control electrode, an input electrode, and an output electrode. For example, the circuit layer DP-CL may include switching transistors and driving transistors for driving the luminescence devices ED-1, ED-2, and ED-3 of the display device layer DP-ED.

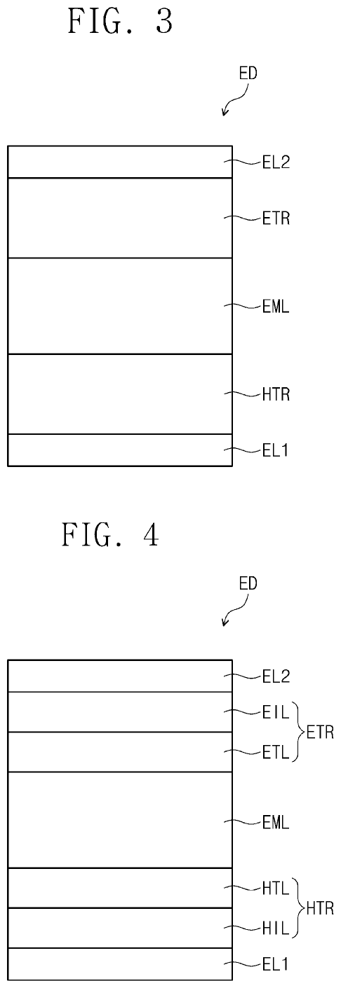

Each of the luminescence devices ED-1, ED-2, and ED-3 may have the structure of a luminescence device ED of an embodiment according to FIG. 3 to FIG. 6, which will be explained later. Each of the luminescence devices ED-1, ED-2, and ED-3 may include a first electrode EL1, a hole transport region HTR, emission layers EML-R, EML-G, and EML-B, an electron transport region ETR, and a second electrode EL2.

In FIG. 2, shown is an embodiment where the emission layers EML-R, EML-G, and EML-B of the luminescence devices ED-1, ED-2, and ED-3, are disposed in opening parts OH defined in the pixel definition layer PDL, and the hole transport region HTR, the electron transport region ETR and the second electrode EL2 are provided as common layers in all luminescence devices ED-1, ED-2, and ED-3. However, embodiments are not limited thereto. Different from what is illustrated in FIG. 2, in an embodiment, the hole transport region HTR and the electron transport region ETR may be patterned and provided in the opening parts OH defined in the pixel definition layer PDL. For example, in an embodiment, the hole transport region HTR, the emission layers EML-R, EML-G, and EML-B, and the electron transport region ETR of the luminescence devices ED-1, ED-2, and ED-3 may be patterned by an ink jet printing method and provided.

The encapsulating layer TFE may cover the luminescence devices ED-1, ED-2, and ED-3. The encapsulating layer TFE may encapsulate the display device layer DP-ED. The encapsulating layer TFE may be a thin film encapsulating layer. The encapsulating layer TFE may be one layer or a stack of multiple layers. The encapsulating layer TFE may include at least one insulating layer. The encapsulating layer TFE according to an embodiment may include at least one inorganic layer (hereinafter, encapsulating inorganic layer). The encapsulating layer TFE according to an embodiment may include at least one organic layer (hereinafter, encapsulating organic layer) and at least one encapsulating inorganic layer.

The encapsulating inorganic layer may protect the display device layer DP-ED from moisture and/or oxygen, and the encapsulating organic layer may protect the display device layer DP-ED from foreign materials such as dust particles. The encapsulating inorganic layer may include silicon nitride, silicon oxy nitride, silicon oxide, titanium oxide, or aluminum oxide, without specific limitation. The encapsulating organic layer may include an acrylic compound, an epoxy-based compound, etc. The encapsulating organic layer may include a photopolymerizable organic material, without specific limitation.

The encapsulating layer TFE may be disposed on the second electrode EL2 and may be disposed to fill an opening part OH.

Referring to FIG. 1 and FIG. 2, the display apparatus DD may include a non-luminous area NPXA and luminous areas PXA-R, PXA-G, and PXA-B. The luminous areas PXA-R, PXA-G, and PXA-B may be areas emitting light produced from the luminescence devices ED-1, ED-2, and ED-3, respectively. The luminous areas PXA-R, PXA-G, and PXA-B may be separated from each other on a plane.

The luminous areas PXA-R, PXA-G, and PXA-B may be areas separated by the pixel definition layer PDL. The non-luminous areas NPXA may be areas between neighboring luminous areas PXA-R, PXA-G, and PXA-B and may be areas corresponding to the pixel definition layer PDL. In the disclosure, each of the luminous areas PXA-R, PXA-G, and PXA-B may each correspond to a pixel. The pixel definition layer PDL may divide the luminescence devices ED-1, ED-2, and ED-3. The emission layers EML-R, EML-G, and EML-B of the luminescence devices ED-1, ED-2, and ED-3 may be disposed and divided in the opening parts OH defined in the pixel definition layer PDL.

The luminous areas PXA-R, PXA-G, and PXA-B may be divided into numbers of groups according to the color of light produced from the luminescence devices ED-1, ED-2, and ED-3. In the display apparatus DD of an embodiment, shown in FIG. 1 and FIG. 2, three luminous areas PXA-R, PXA-G, and PXA-B emitting red light, green light, and blue light are illustrated as an embodiment. For example, the display apparatus DD of an embodiment may include a red luminous area PXA-R, a green luminous area PXA-G, and a blue luminous area PXA-B, which are separated from each other.

In the display apparatus DD according to an embodiment, multiple luminescence devices ED-1, ED-2, and ED-3 may emit light having different wavelength regions. For example, in an embodiment, the display apparatus DD may include a first luminescence device ED-1 emitting red light, a second luminescence device ED-2 emitting green light, and a third luminescence device ED-3 emitting blue light. For example, each of the red luminous area PXA-R, the green luminous area PXA-G, and the blue luminous area PXA-B of the display apparatus DD may respectively correspond to the first luminescence device ED-1, the second luminescence device ED-2, and the third luminescence device ED-3.

However, embodiments are not limited thereto, and the first to third luminescence devices ED-1, ED-2, and ED-3 may emit light in the same wavelength region, or at least one thereof may emit light in a different wavelength region. For example, all the first to third luminescence devices ED-1, ED-2, and ED-3 may emit blue light.

The luminous areas PXA-R, PXA-G, and PXA-B in the display apparatus DD according to an embodiment may be arranged in a stripe shape. Referring to FIG. 1, multiple red luminous areas PXA-R, multiple green luminous areas PXA-G, and multiple blue luminous areas PXA-B may be arranged along a second directional axis DR2. The red luminous area PXA-R, the green luminous area PXA-G, and the blue luminous area PXA-B may be arranged by turns along a first directional axis DR1.

In FIG. 1 and FIG. 2, the areas of the luminous areas PXA-R, PXA-G, and PXA-B are shown similar, but embodiments are not limited thereto. The areas of the luminous areas PXA-R, PXA-G, and PXA-B may be different from each other according to the wavelength region of light emitted. For example, the areas of the luminous areas PXA-R, PXA-G, and PXA-B may be areas in a plan view that are defined by the first directional axis DR1 and the second directional axis DR2.

The arrangement type of the luminous areas PXA-R, PXA-G, and PXA-B is not limited to the configuration shown in FIG. 1, and the arrangement order of the red luminous areas PXA-R, the green luminous areas PXA-G, and the blue luminous areas PXA-B may be provided in various combinations according to the properties of display quality required for the display apparatus DD. For example, the arrangement type of the luminous areas PXA-R, PXA-G, and PXA-B may be a PenTile® arrangement type, or a diamond arrangement type.

The areas of the luminous areas PXA-R, PXA-G, and PXA-B may be different from each other. For example, in an embodiment, the area of the green luminous area PXA-G may be smaller than the area of the blue luminous area PXA-B, but embodiments are not limited thereto.

Hereinafter, FIG. 3 to FIG. 6 are schematic cross-sectional views each showing luminescence devices according to embodiments. The luminescence device ED according to an embodiment may include a first electrode EL1, a hole transport region HTR, an emission layer EML, an electron transport region ETR, and a second electrode EL2, stacked in that order.

The luminescence device ED of an embodiment includes a monoamine compound of an embodiment, which will be explained later, in a hole transport region HTR disposed between a first electrode EL1 and a second electrode EL2. However, embodiments are not limited thereto, and the luminescence device ED of an embodiment may include a compound according to an embodiment, which will be explained later, in an emission layer EML, which may include multiple functional layers disposed between a first electrode EL1 and a second electrode EL2, or in an electron transport region ETR in addition to the hole transport region HTR, or may include a compound according to an embodiment, which will be explained later, in a capping layer CPL disposed on a second electrode EL2.

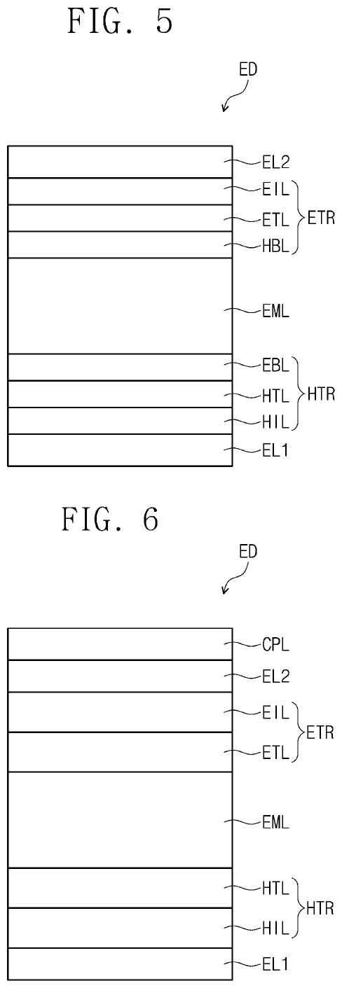

In comparison to FIG. 3, FIG. 4 shows a schematic cross-sectional view of a luminescence device ED of an embodiment, wherein a hole transport region HTR includes a hole injection layer HIL and a hole transport layer HTL, and an electron transport region ETR includes an electron injection layer EIL and an electron transport layer ETL. In comparison to FIG. 3, FIG. 5 shows a schematic cross-sectional view of a luminescence device ED of an embodiment, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. In comparison to FIG. 4, FIG. 6 shows a schematic cross-sectional view of a luminescence device ED of an embodiment, including a capping layer CPL disposed on the second electrode EL2.

The first electrode EL1 has conductivity. The first electrode EL1 may be formed using a metal alloy or a conductive compound. The first electrode EL1 may be an anode or a cathode. However, embodiments are not limited thereto. In an embodiment, the first electrode EL1 may be a pixel electrode. The first electrode EL1 may be a transmissive electrode, a transflective electrode, or a reflective electrode. If the first electrode EL1 is a transmissive electrode, the first electrode EL1 may be formed using a transparent metal oxide such as indium tin oxide (ITO), indium zinc oxide (IZO), zinc oxide (ZnO), and indium tin zinc oxide (ITZO). If the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca, LiF/Al, Mo, Ti, compounds thereof, or mixtures thereof (for example, a mixture of Ag and Mg). In another embodiment, a structure including multiple layers including a reflective layer or a transflective layer formed using the above materials, and a transmissive conductive layer formed using ITO, IZO, ZnO, or ITZO may be formed. For example, the first electrode EL1 may include a three-layer structure of ITO/Ag/ITO. However, embodiments are not limited thereto. A thickness of the first electrode EL1 may be in a range of about 700 Å to about 10,000 Å. For example, the thickness of the first electrode EL1 may be in a range of about 1,000 Å to about 3,000 Å.

The hole transport region HTR is provided on the first electrode ELL The hole transport region HTR may include at least one of a hole injection layer HIL, a hole transport layer HTL, a hole buffer layer (not shown), and an electron blocking layer EBL. A thickness of the hole transport region HTR may be in a range of about 50 Å to about 15,000 Å.

The hole transport region HTR may have a single layer formed using a single material, a single layer formed using different materials, or a multilayer structure including multiple layers formed using different materials.

For example, the hole transport region HTR may have the structure of a single layer of a hole injection layer HIL or a hole transport layer HTL, and may have a structure of a single layer formed using a hole injection material and a hole transport material. In another embodiment, the hole transport region HTR may have a structure of a single layer formed using multiple different materials, or a structure stacked from the first electrode EL1 of hole injection layer HIL/hole transport layer HTL, hole injection layer HIL/hole transport layer HTL/buffer layer (not shown), hole injection layer HIL/buffer layer (not shown), hole transport layer HTL/buffer layer, or hole injection layer HIL/hole transport layer HTL/electron blocking layer EBL, without limitation.

The hole transport region HTR of the luminescence device ED of an embodiment includes a monoamine compound according to an embodiment.

In the description, the term “substituted or unsubstituted” corresponds to substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkoxy group, a hydrocarbon ring group, an aryl group, and a heterocyclic group. Each of the substituents may be substituted or unsubstituted. For example, a biphenyl group may be interpreted as an aryl group or a phenyl group substituted with a phenyl group.

In the description, the term “bonded to an adjacent group to form a ring” may indicate that one is bonded to an adjacent group to form a substituted or unsubstituted hydrocarbon ring, or a substituted or unsubstituted heterocycle. The hydrocarbon ring may include an aliphatic hydrocarbon ring and an aromatic hydrocarbon ring. The heterocycle may include an aliphatic heterocycle and an aromatic heterocycle. Rings formed by being bonded to an adjacent group may be monocyclic or polycyclic. The rings formed by being bonded to each other may be connected to another ring to form a spiro structure.

In the description, the term “an adjacent group” may mean a substituent substituted for an atom which is directly connected to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as mutually “adjacent groups” and two ethyl groups in 1,1-diethylcyclopentane may be interpreted as mutually “adjacent groups”.

In the description, the halogen atom may be a fluorine atom, a chlorine atom, a bromine atom, or an iodine atom.

In the description, the alkyl group may be a linear, branched, or cyclic type. The number of carbon atoms in an alkyl group may be, for example, 1 to 50, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methyiheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the description, the alkenyl group means a hydrocarbon group including one or more carbon double bonds in the middle of or at the terminal of an alkyl group of 2 or more carbon atoms. The alkenyl group may be a linear chain or a branched chain. The number of carbon atoms in an alkenyl group is not specifically limited, but may be 2 to 30, 2 to 20 or 2 to 10. Examples of the alkenyl group may include a vinyl group, a 1-butenyl group, a 1-pentenyl group, a 1,3-butadienyl aryl group, a styrenyl group, a styrylvinyl group, etc., without limitation.

In the description, the alkynyl group means a hydrocarbon group including one or more carbon triple bonds in the middle of or at the terminal of an alkyl group of 2 or more carbon atoms. The alkynyl group may be a linear chain or a branched chain. The number of carbon atoms in an alkynyl is not specifically limited, but may be 2 to 30, 2 to 20 or 2 to 10. Examples of the alkenyl group may include an ethynyl group, a propynyl group, etc., without limitation.

In the description, the hydrocarbon ring group means an optional functional group or substituent derived from an aliphatic hydrocarbon ring, or an optional functional group or substituent derived from an aromatic hydrocarbon ring. The number of ring-forming carbon atoms in the hydrocarbon ring group may be 5 to 60, 5 to 30, or 5 to 20.

In the description, the aryl group means an optional functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include phenyl, naphthyl, fluorenyl, anthracenyl, phenanthryl, biphenyl, terphenyl, quaterphenyl, quinqphenyl, sexiphenyl, triphenylenyl, pyrenyl, benzofluoranthenyl, chrysenyl, etc., without limitation.

In the description, the fluorenyl group may be substituted, and two substituents may be combined with each other to form a spiro structure. Examples of a substituted fluorenyl group are as follows. However, embodiments are not limited thereto.

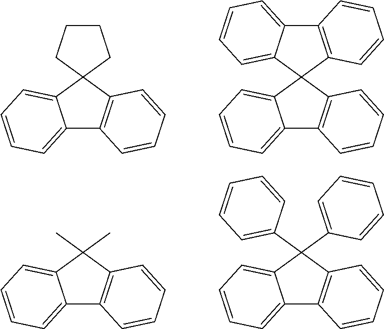

In the description, the heterocyclic group means an optional functional group or substituent derived from a ring including one or more among B, O, N, P, Si, and S as heteroatoms. The heterocyclic group includes an aliphatic heterocyclic group and an aromatic heterocyclic group. The aromatic heterocyclic group may be a heteroaryl group. The aliphatic heterocyclic group and the aromatic heterocyclic group may be a monocycle or a polycycle.

In the description, the heterocyclic group may include one or more among B, O, N, P, Si, and S as heteroatoms. If the heterocyclic group includes two or more heteroatoms, two or more heteroatoms may be the same or different. The heterocyclic group may be a monocyclic heterocyclic group or a polycyclic heterocyclic group, and has the concept including a heteroaryl group. The number of ring-forming carbon atoms of the heteroaryl group may be 2 to 30, 2 to 20, 2 to 12, or 2 to 10.

In the description, the aliphatic heterocyclic group may include one or more among B, O, N, P, Si, and S as heteroatoms. The number of ring-forming carbon atoms of the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., without limitation.

In the description, the heteroaryl group may include one or more among B, O, N, P, Si, and S as heteroatoms. If the heteroaryl group includes two or more heteroatoms, two or more heteroatoms may be the same or different. The heteroaryl group may be a monocyclic heteroaryl group or polycyclic heteroaryl group. The number of ring-forming carbon atoms in the heteroaryl group may be 2 to 30, 2 to 20, or 2 to 10. Examples of the heteroaryl group may include thiophene, furan, pyrrole, imidazole, triazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofurane, phenanthroline, thiazole, isooxazole, oxazole, oxadiazole, thiadiazole, phenothiazine, dibenzosilole, dibenzofuran, etc., without limitation.

In the description, the number of carbon atoms in an amine group is not specifically limited, but may be 1 to 30. The amine group may include an alkyl amine group, an aryl amine group, or a heteroaryl amine group. Examples of the amine group may include a methylamine group, a dimethylamine group, a phenylamine group, a diphenylamine group, a naphthylamine group, a 9-methyl-anthracenylamine group, etc., without limitation.

In the description, the explanation on the aryl group may be applied to the arylene group except that the arylene group is a divalent group.

The explanation on the heteroaryl group may be applied to the heteroarylene group except that the heteroarylene group is a divalent group.

In the description, “-*” and “*” each indicate a binding site to a neighboring atom.

The amine compound according to an embodiment may be represented by Formula 1 below.

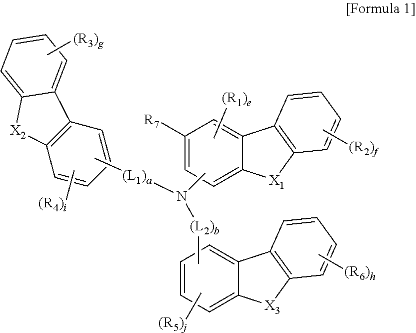

In Formula 1, X₁, X₂, and X₃ may each independently be O or S.

In Formula 1, R₁ to R₆ may each independently be a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, or may be combined with an adjacent group to form a ring.

In Formula 1, R₇ may be a hydrogen atom, a deuterium atom, a halogen atom, or a substituted or unsubstituted alkyl group of 1 to 20 carbon atoms.

In Formula 1, L₁ and L₂ may each independently be a substituted or unsubstituted arylene group of 6 to 30 ring-forming carbon atoms, where L₁ and L₂ do not include a heteroaryl group.

In Formula 1, a and b may each independently be an integer from 1 to 3. If a is 2 or more, then multiple L₁ groups may be the same or different, and if b is 2 or more, then multiple L₂ groups may be the same or different.

In Formula 1, e may be an integer from 0 to 2. If e is 2 or more, then multiple R₁ groups may be the same or different.

In Formula 1, f to h may each independently be an integer from 0 to 4. If f is 2 or more, then multiple R₂ groups may be the same or different, if g is 2 or more, then multiple R₃ groups may be the same or different, and if h is 2 or more, then multiple R₄ groups may be the same or different.

In Formula 1, i and j may each independently be an integer from 0 to 3. If i is 2 or more, then multiple R₅ groups may be the same or different, and if j is 2 or more, then multiple R₆ groups may be the same or different.

However, X₁, X₂, and X₃ are not O at the same time.

In an embodiment, X₁ of Formula 1 may be S, and thus, Formula 1 may be represented by Formula 2 below.

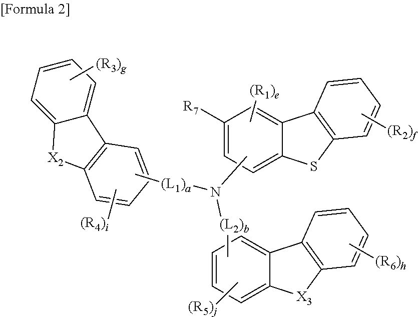

In Formula 2, X₂, X₃, R₁ to R₇, L₁, L₂, a, b, and e to j may be the same as defined in connection with Formula 1.

In an embodiment, X₁ of Formula 1 may be S, and X₂ and X₃ of Formula 1 may each be O.

In an embodiment, X₁ and X₃ of Formula 1 may each be S, and X₂ may be 0.

In an embodiment, X₁ to X₃ of Formula 1 may each be S.

In an embodiment, X₁ of Formula 1 may be 0, and thus, Formula 1 may be represented by Formula 3 below.

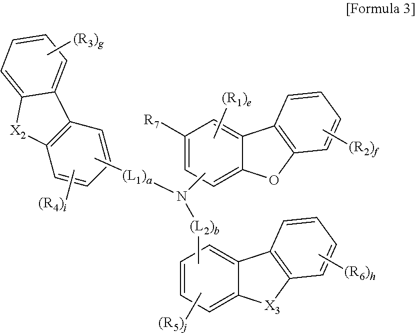

In Formula 3, X₂, X₃, R₁ to R₇, L₁, L₂, a, b, and e to j may be the same as defined in connection with Formula 1.

In an embodiment, X₁ and X₂ of Formula 1 may each be O, and X₃ may be S.

In an embodiment, X₁ of Formula 1 may be 0, and X₂ and X₃ may each be S.

In an embodiment, Formula 2 may be represented by any one among Formula 4-1 to Formula 4-3 below.

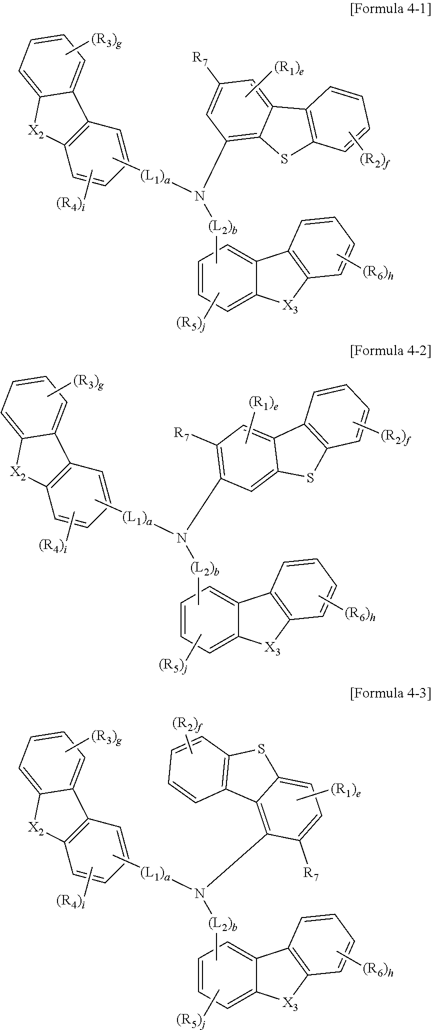

In Formula 4-1 to Formula 4-3, X₂, X₃, R₁ to R₇, L₁, L₂, a, b, and e to j may be the same as defined in connection with Formula 2.

In an embodiment, Formula 3 may be represented by any one among Formula 5-1 to Formula 5-3 below.

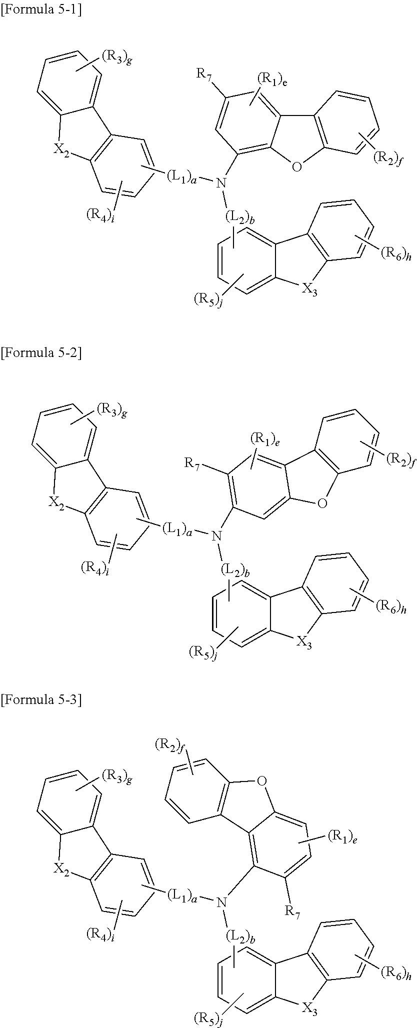

In Formula 5-1 to Formula 5-3, X₂, X₃, R₁ to R₇, L₁, L₂, a, b, and e to j may be the same as defined in connection with Formula 3.

In an embodiment, in any one formula among Formula 1 to Formula 5-3, a and b may each be 1, and L₁ and L₂ may each independently be a substituted or unsubstituted phenylene group, a substituted or unsubstituted biphenylene group, a substituted or unsubstituted terphenyl group, a substituted or unsubstituted naphthylene group, or a substituted or unsubstituted phenanthrylene group.

In an embodiment, in any one formula among Formula 1 to Formula 5-3, a and b may each be 1, and L₁, and L₂ may each independently be represented by any one among L-1 to L-11 below.

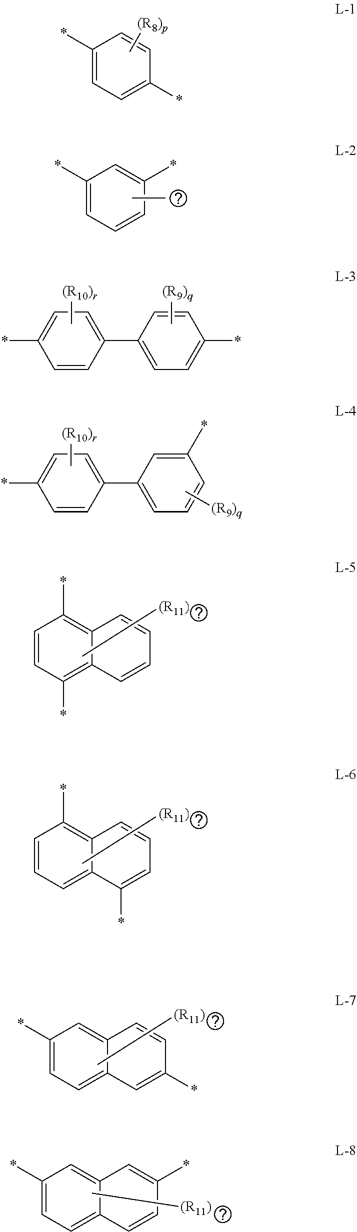

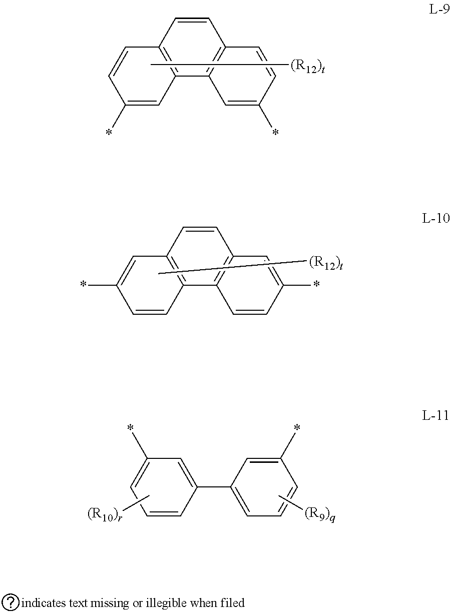

In L-1 to L-11, R₈ to R₁₂ may each independently be a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms. In L-1 to L-11, * indicates a bonding site to a neighboring atom. In L-1 to L-4 and L-11, p to r may each independently be an integer from 0 to 4. If p is 2 or more, then multiple R₈ groups may be the same or different, if q is 2 or more, then multiple R₉ groups may be the same or different, and if r is 2 or more, then multiple R₁₀ groups may be the same or different.

In L-5 to L-8, s may be an integer from 0 to 6. If s is 2 or more, then multiple R₁₁ groups may be the same or different.

In L-9 to L-10, t may be an integer from 0 to 8. If t is 2 or more, then multiple R₁₂ groups may be the same or different.

The amine compound represented by Formula 1 according to an embodiment may be any one selected from the compounds represented in Compound Groups 1 and 2 below. However, embodiments are not limited thereto.

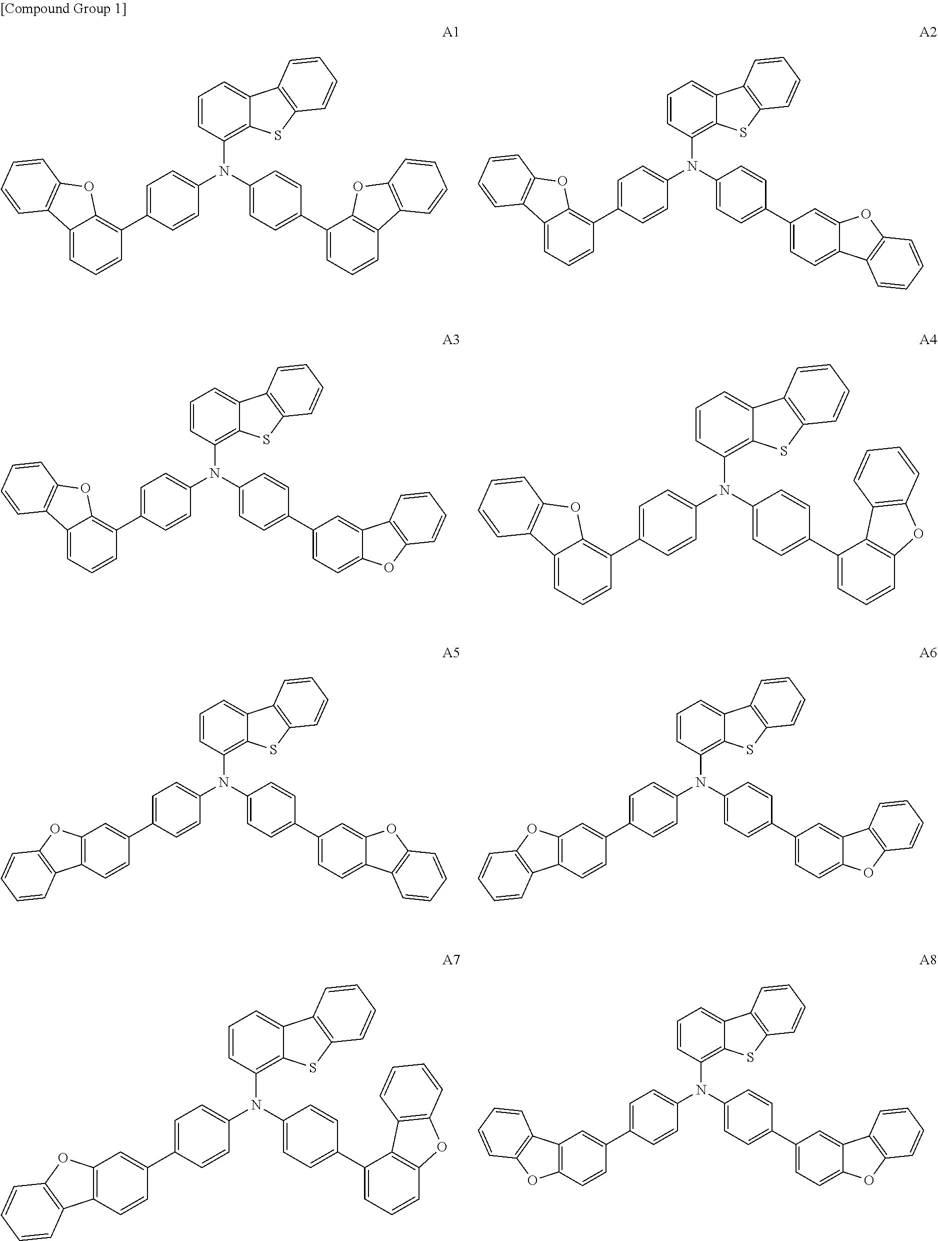

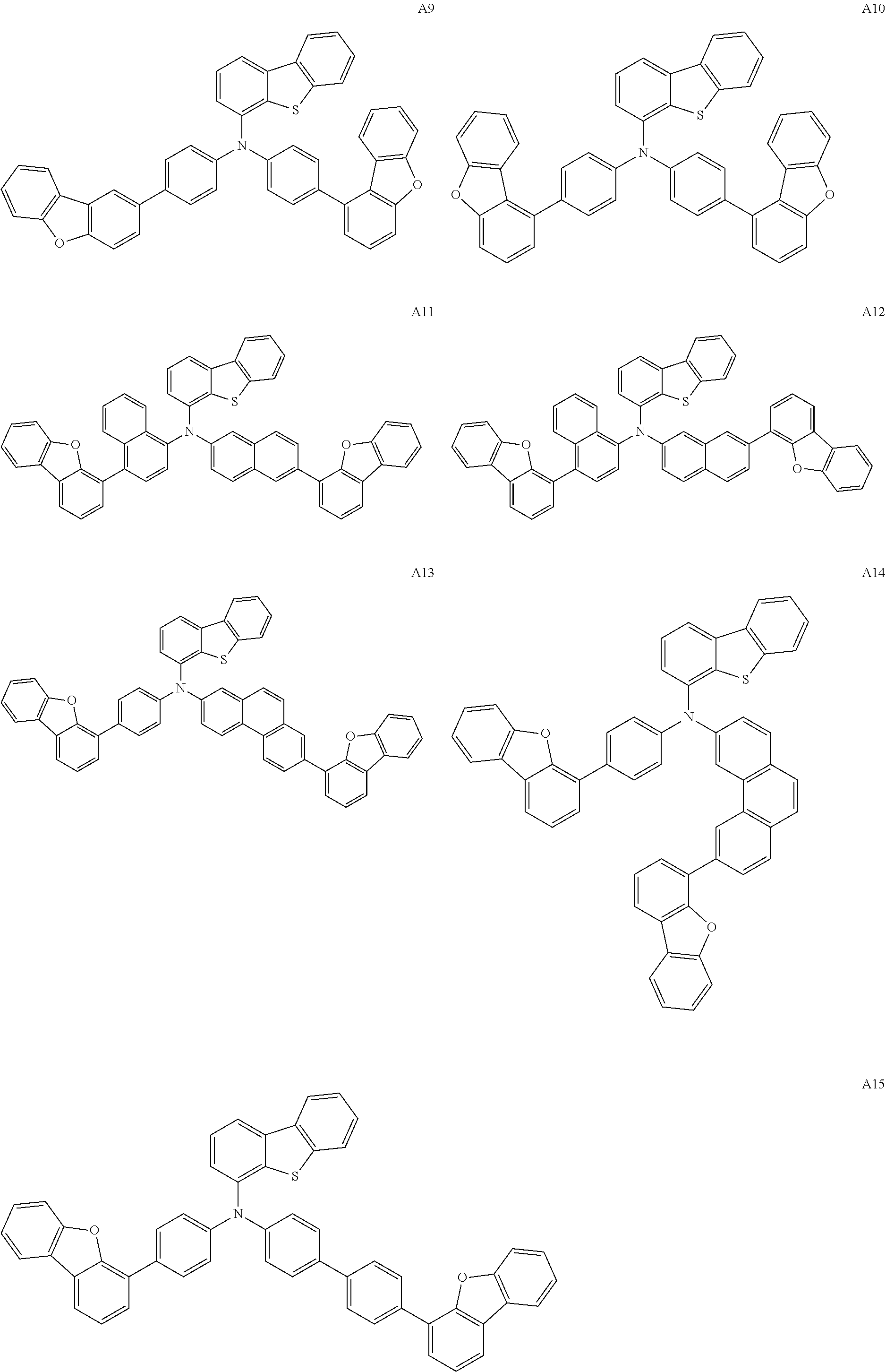

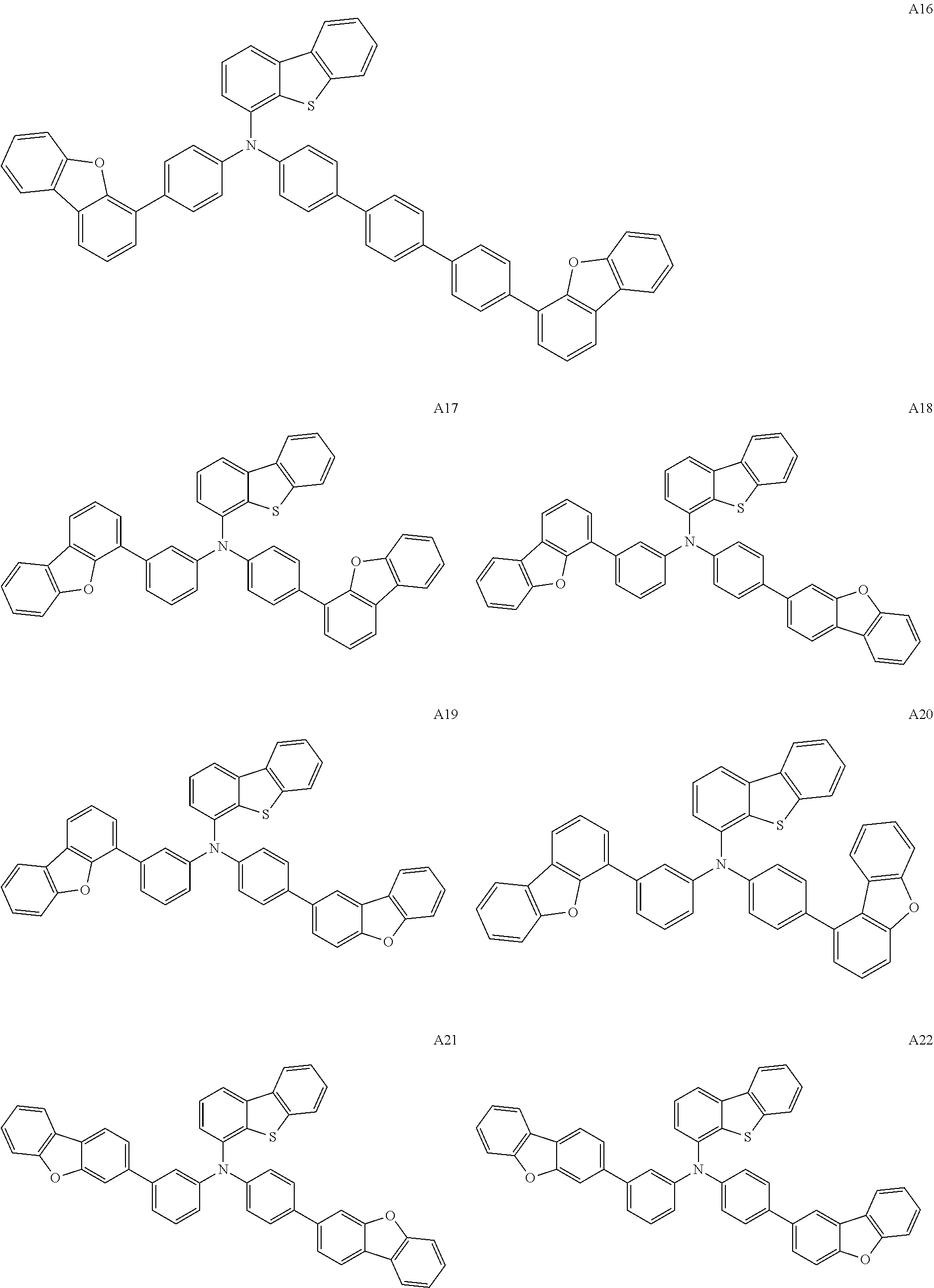

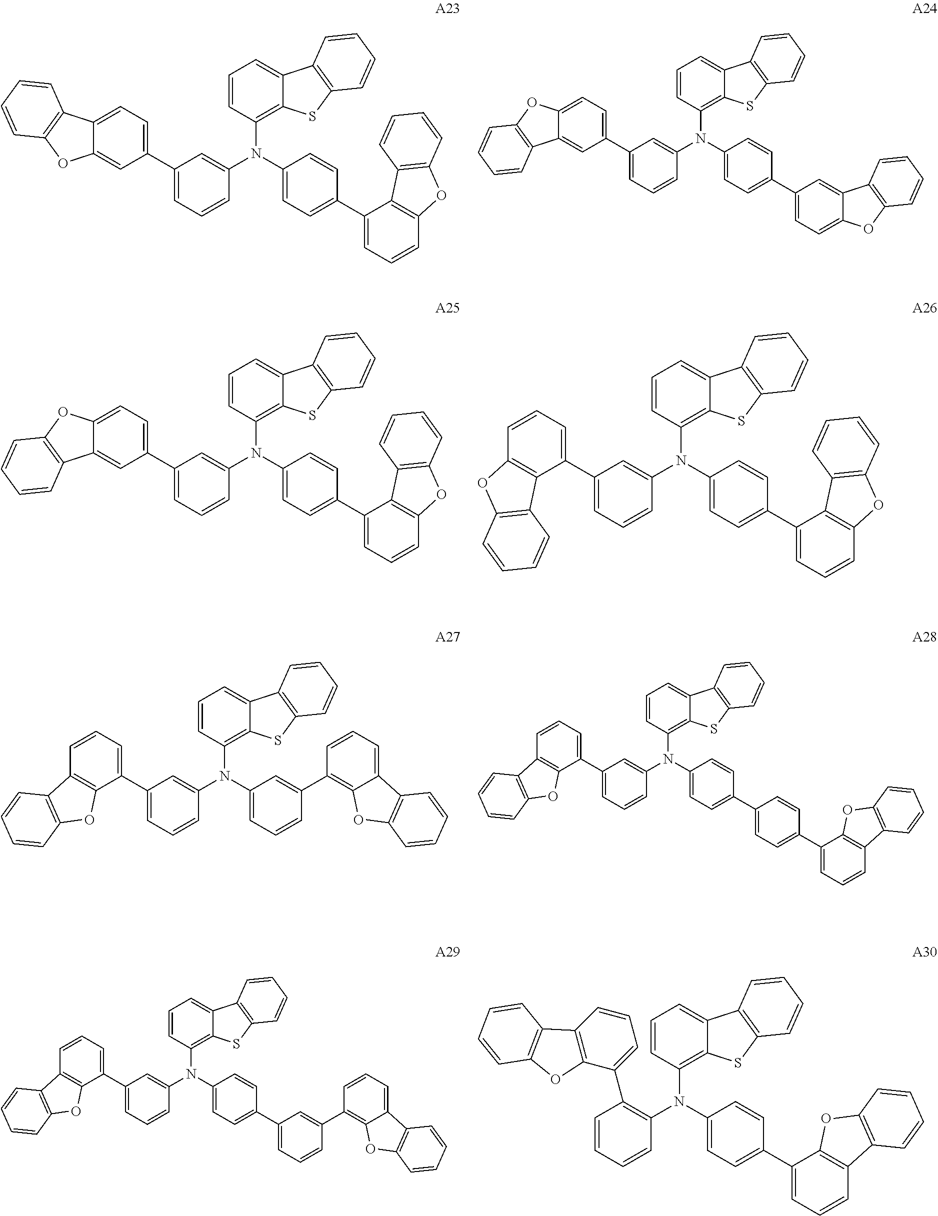

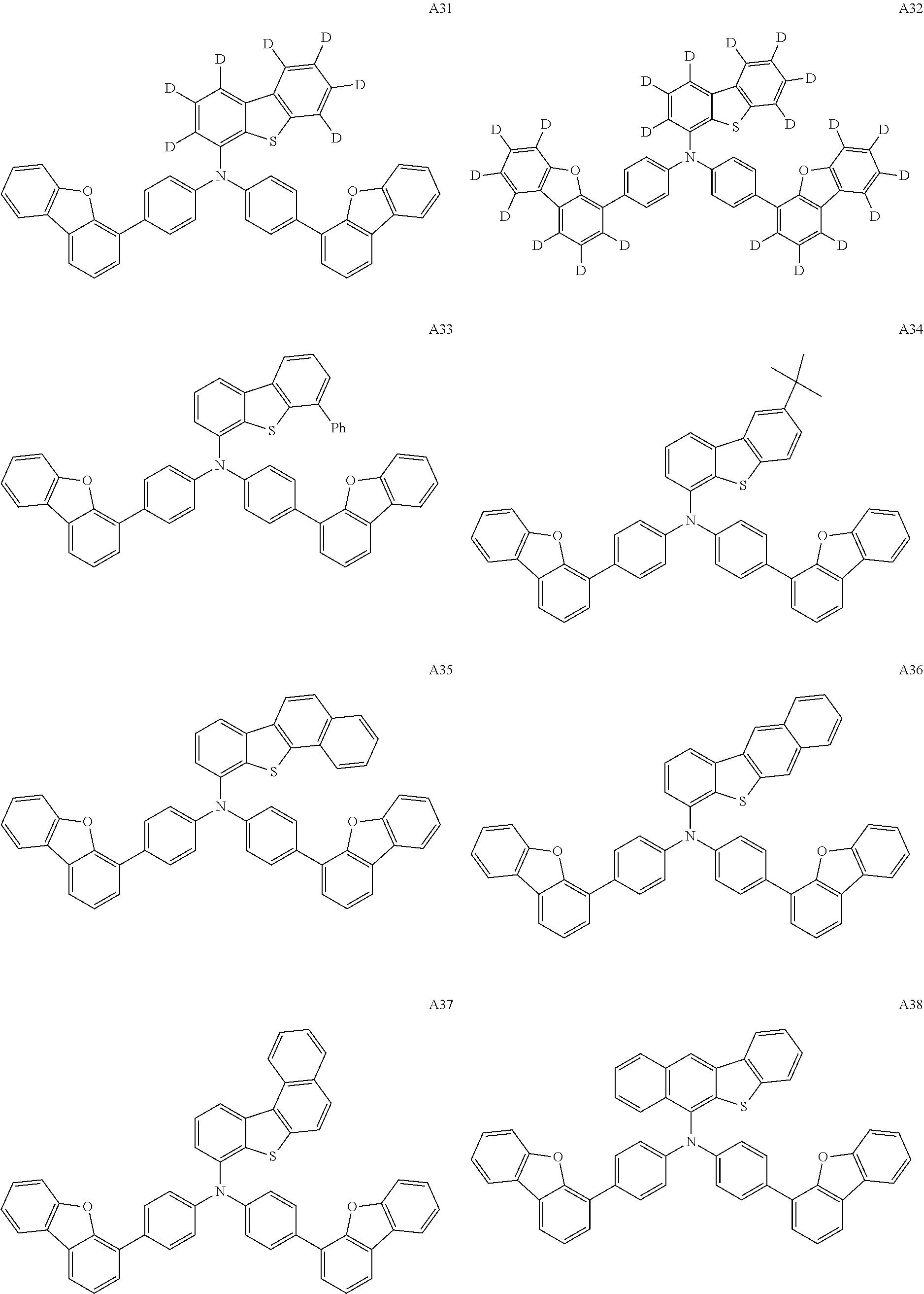

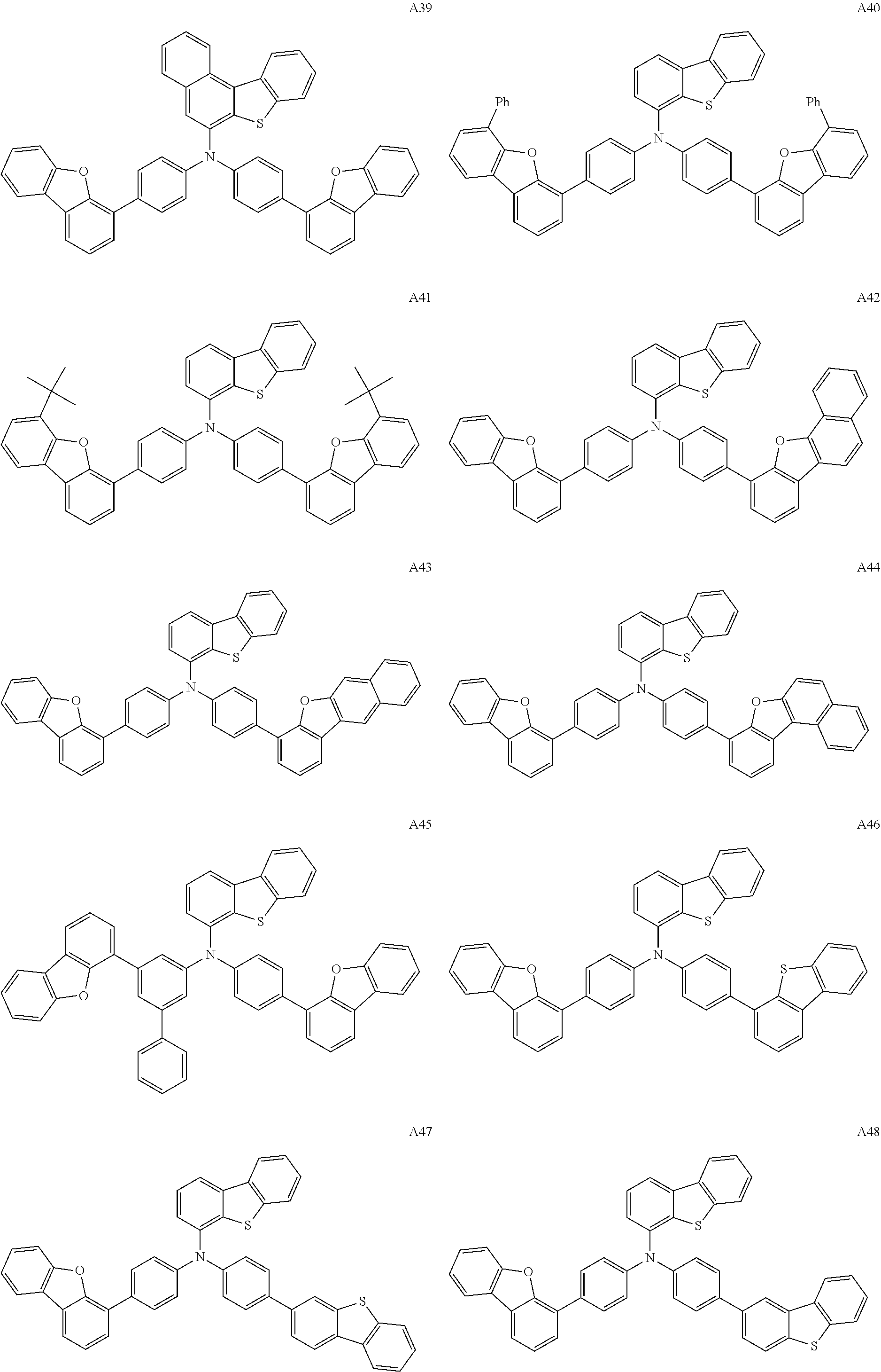

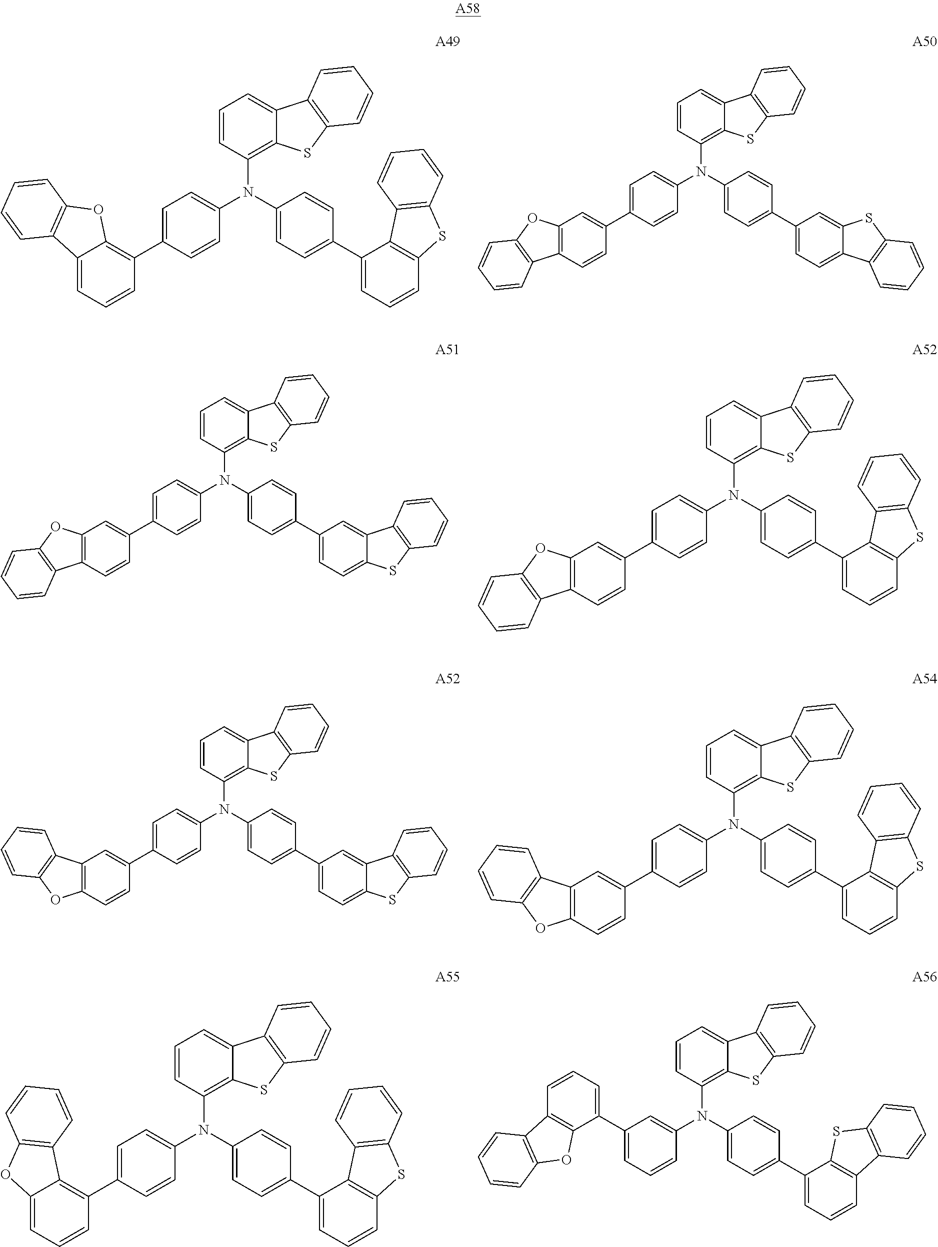

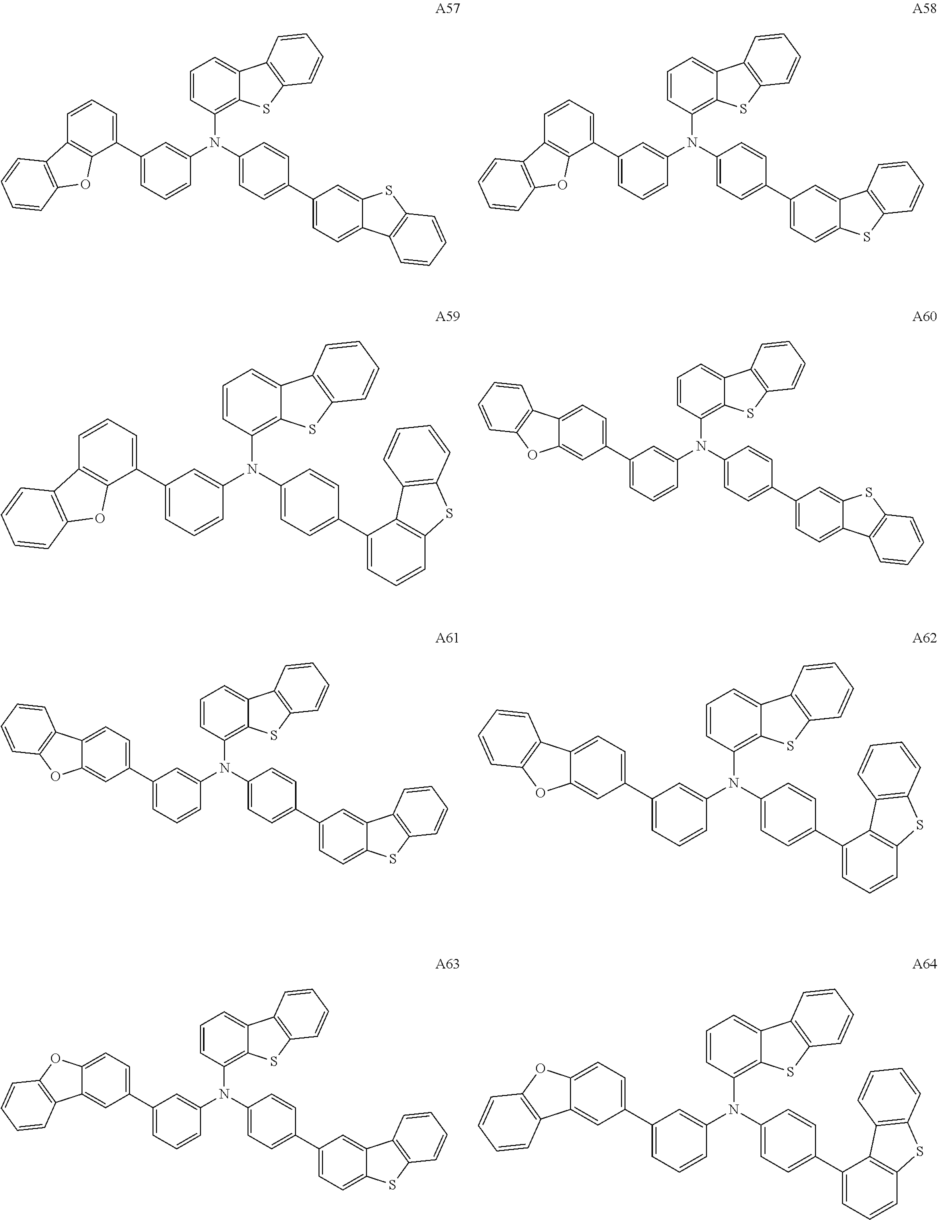

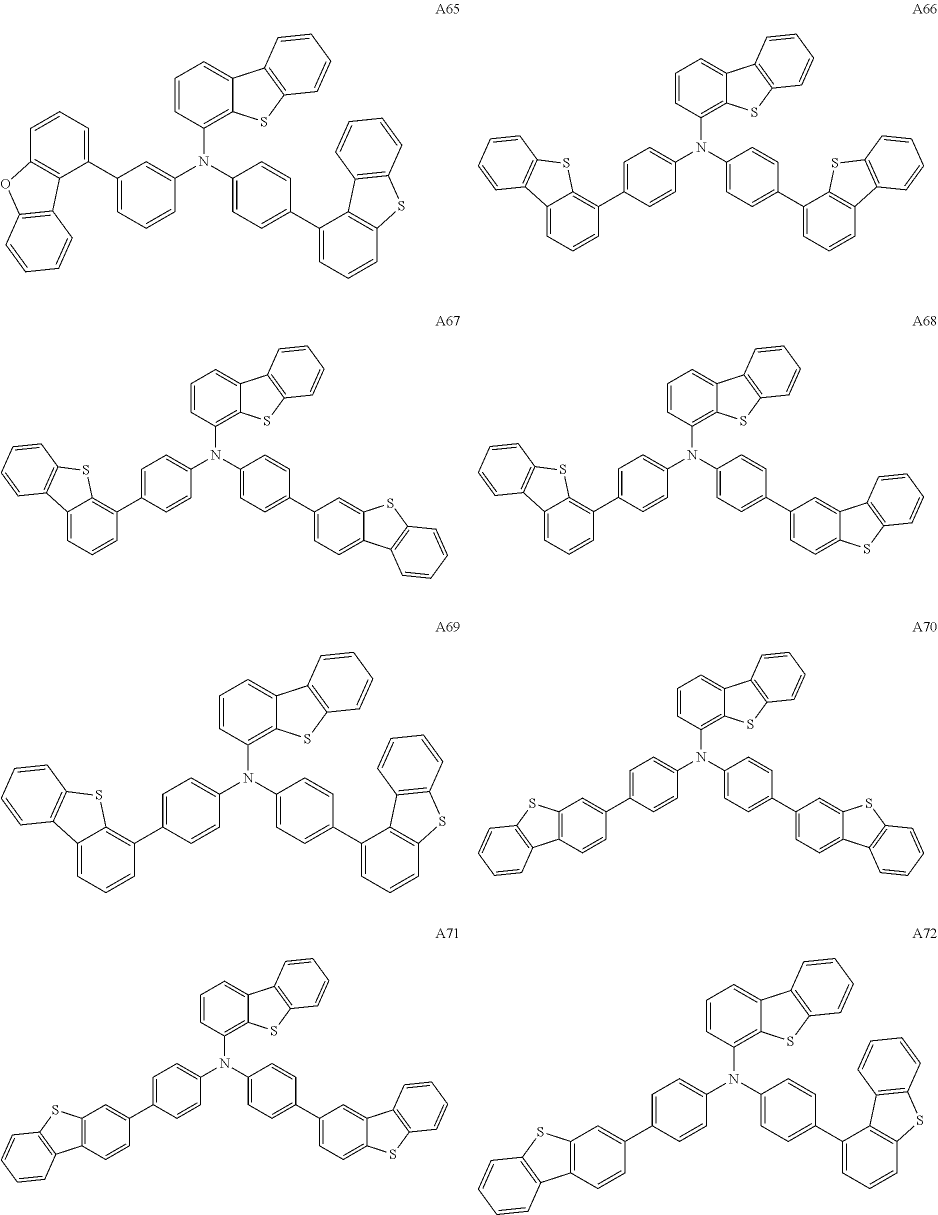

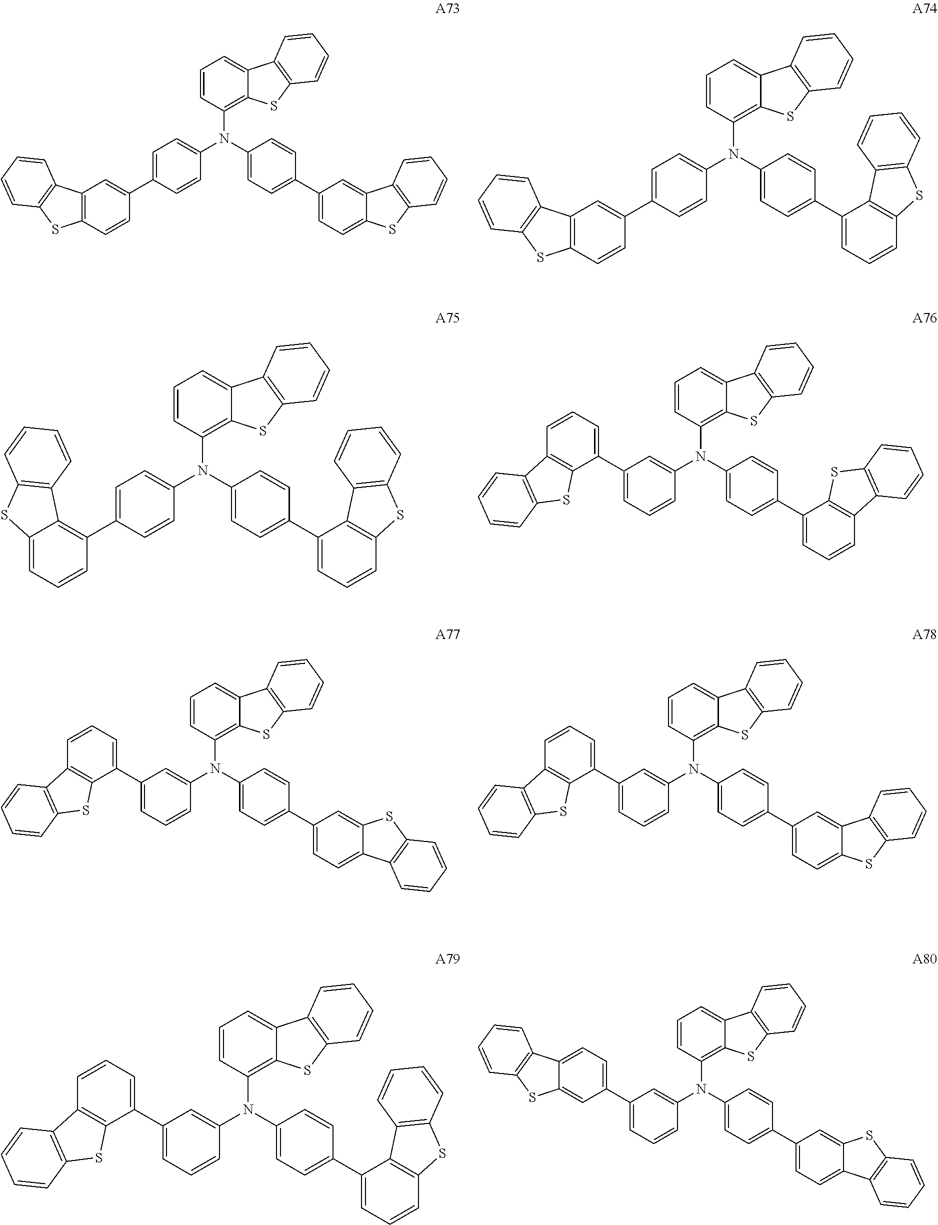

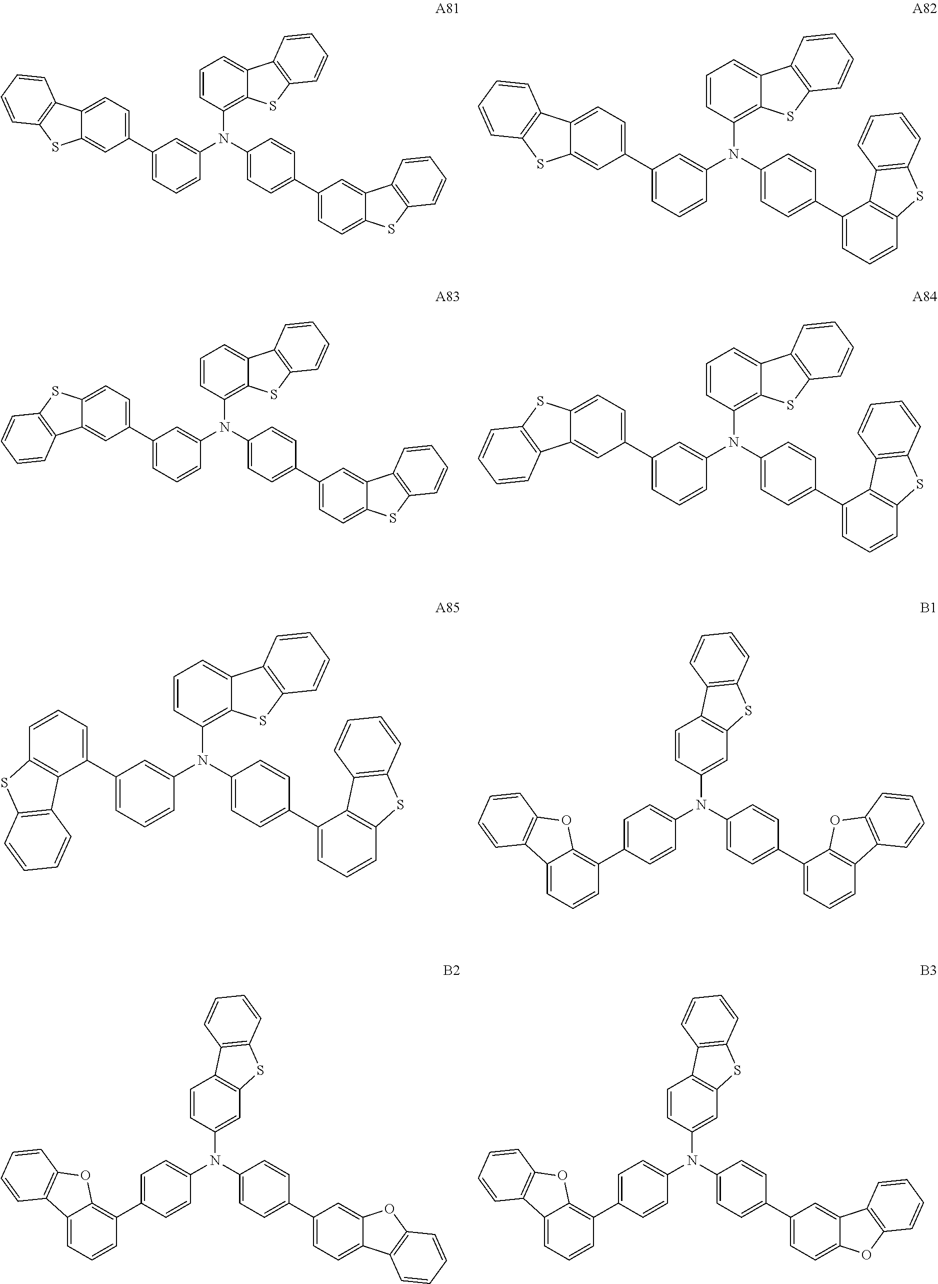

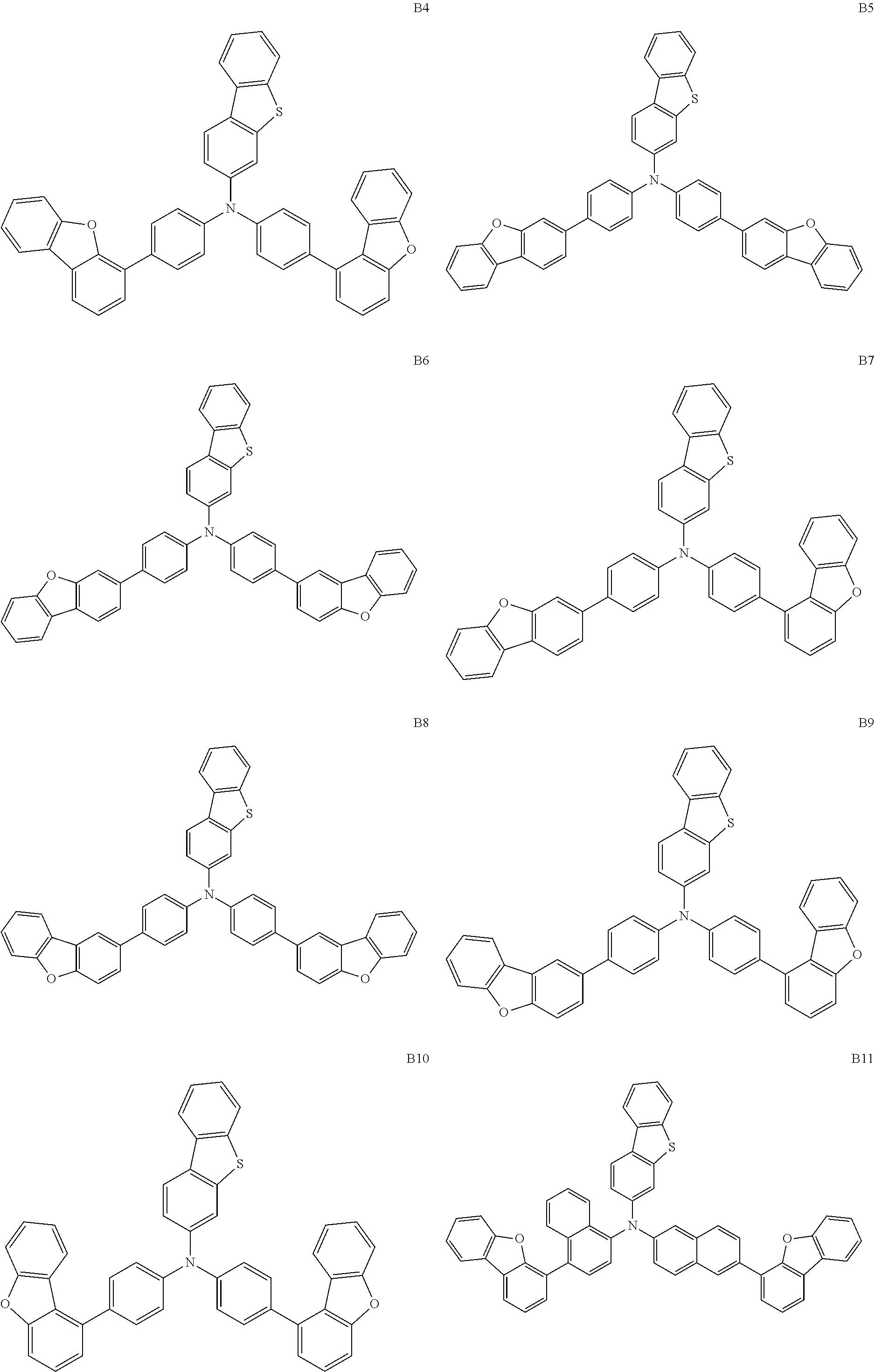

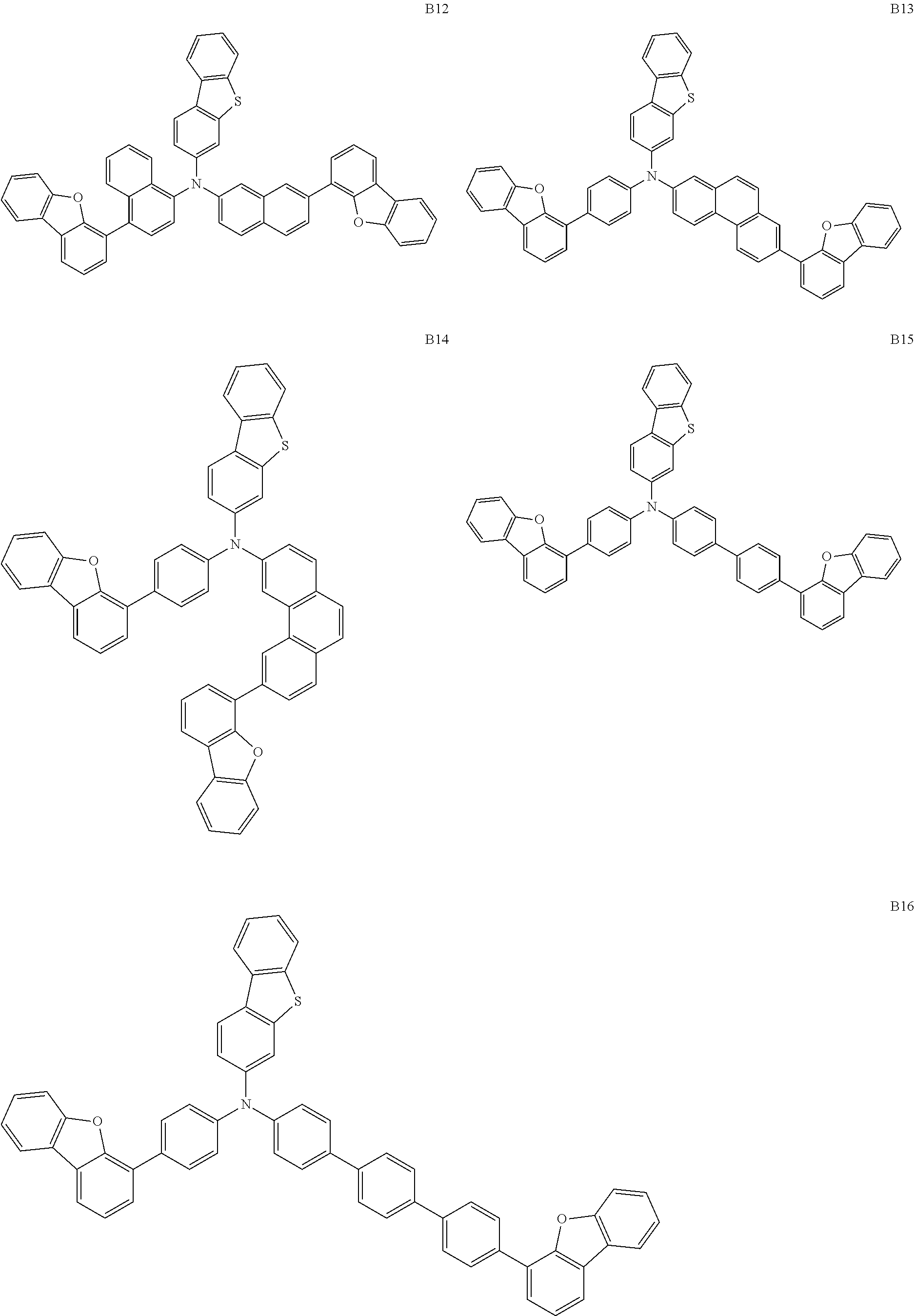

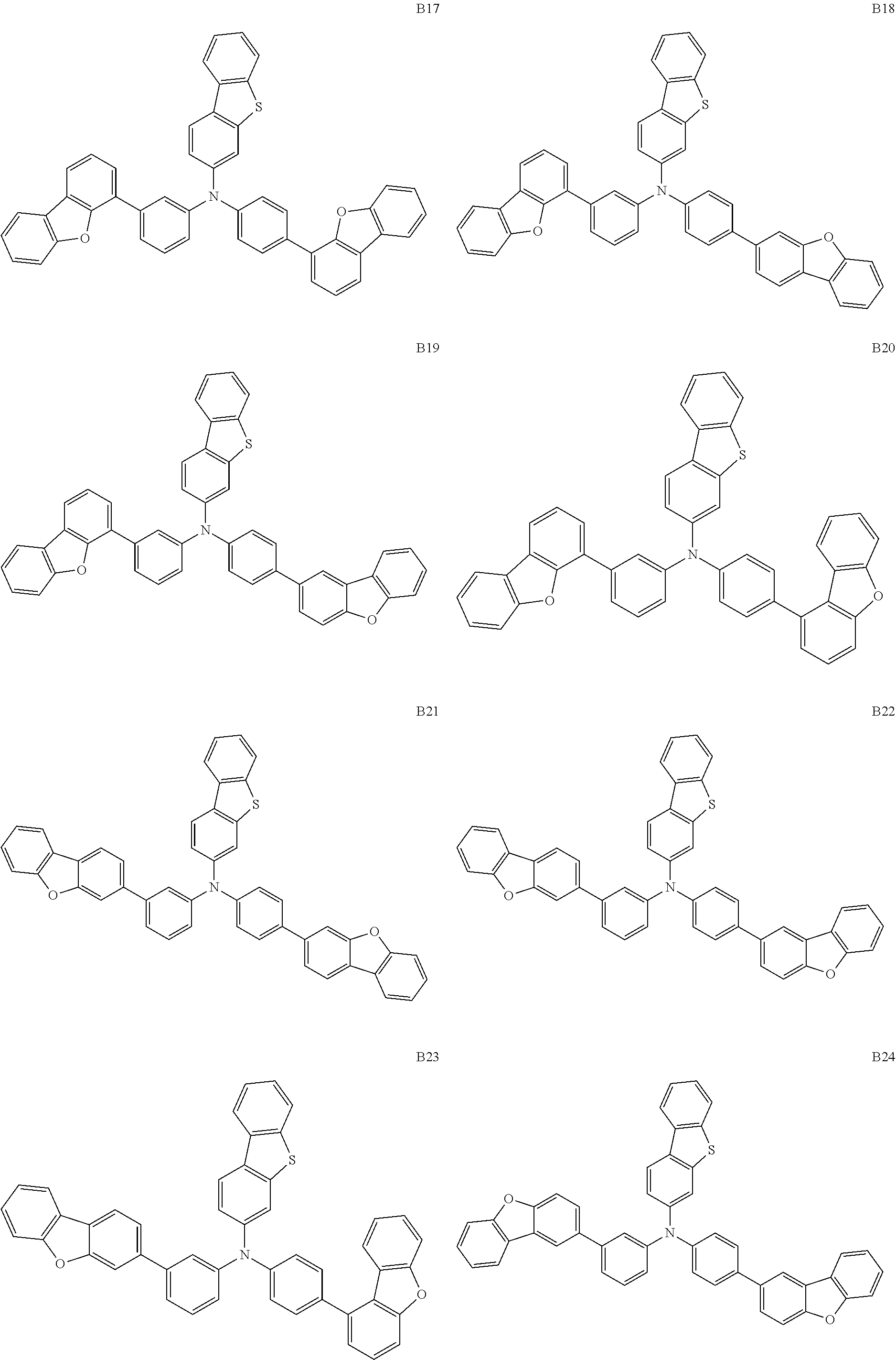

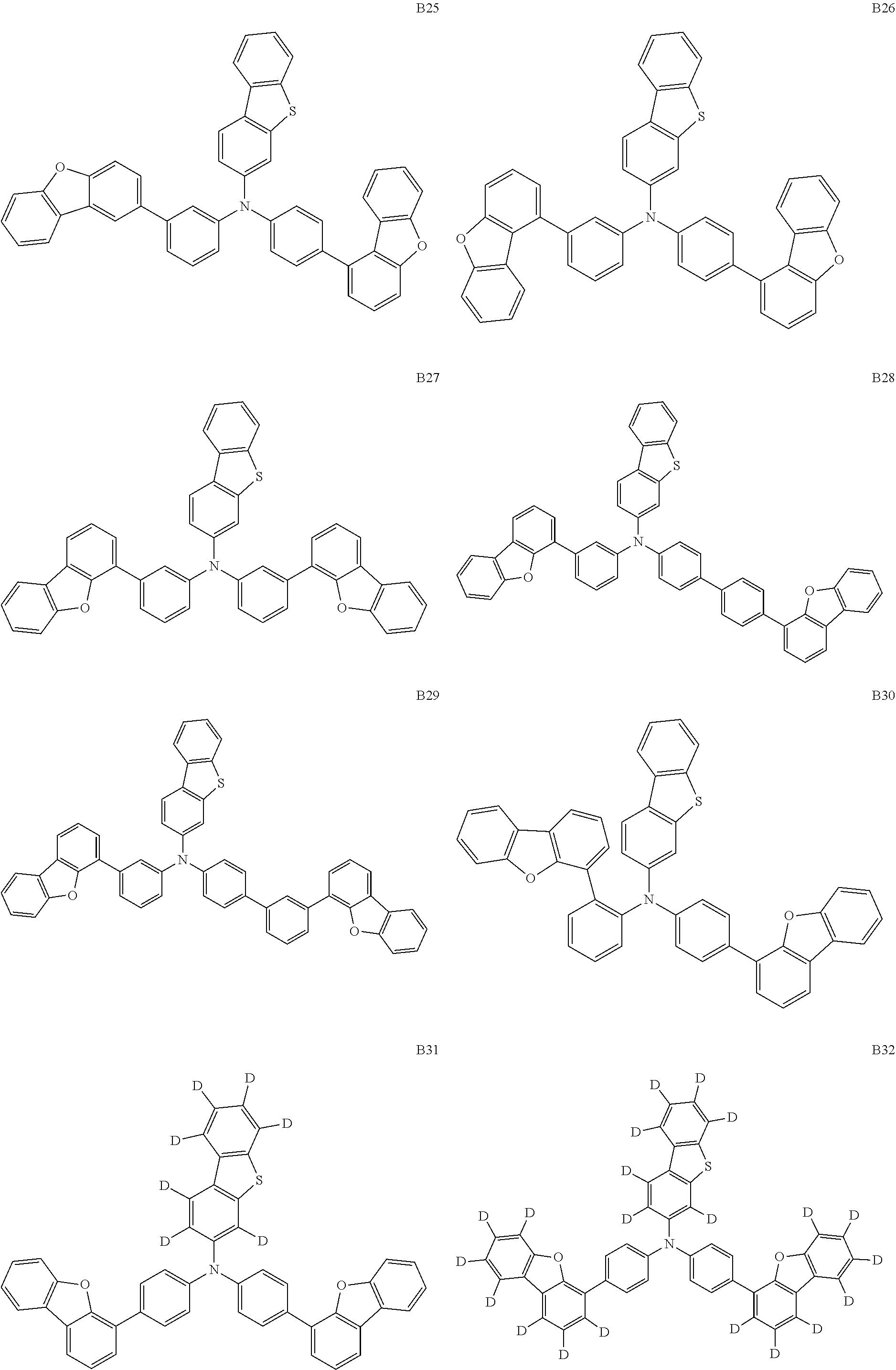

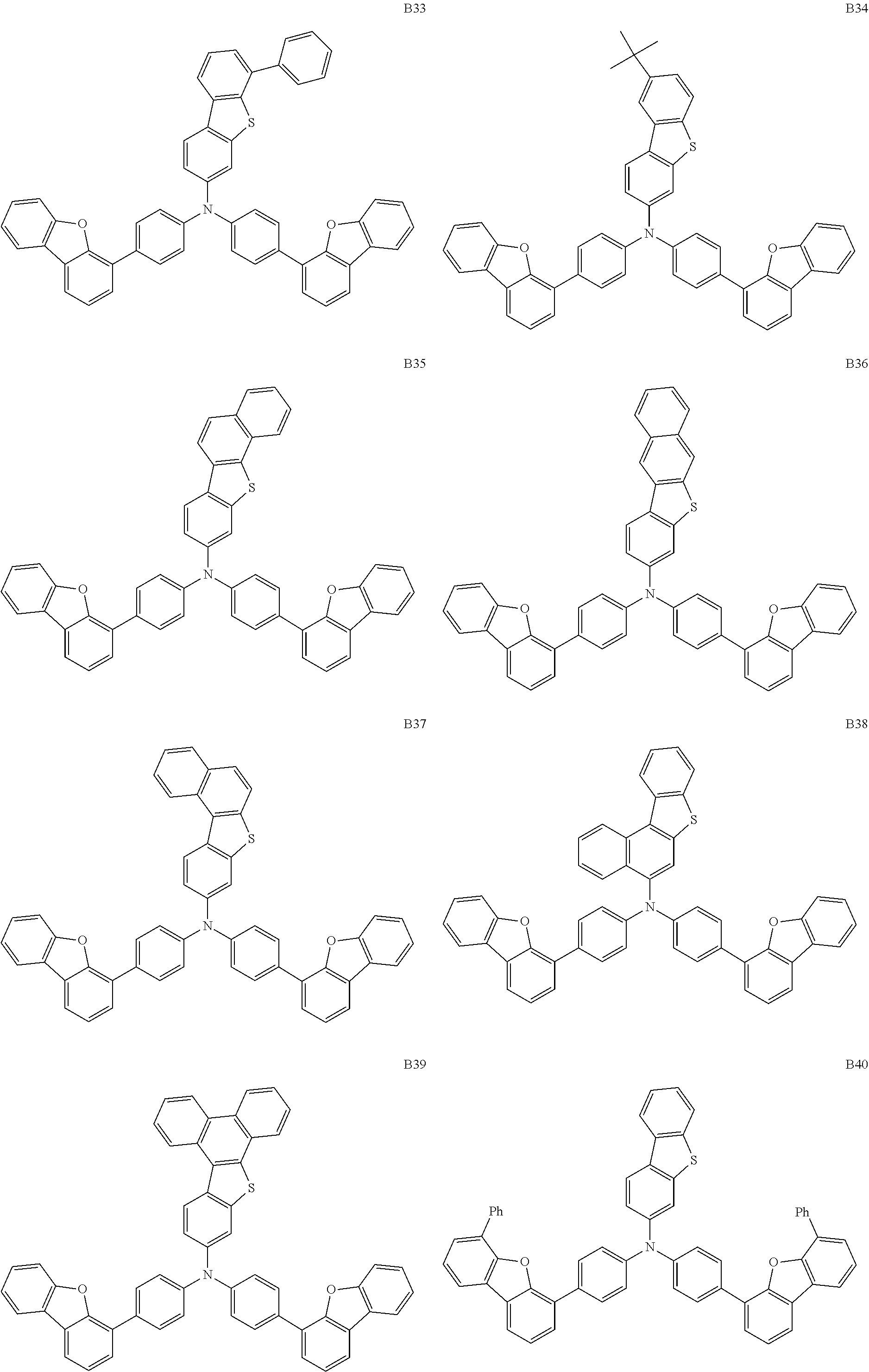

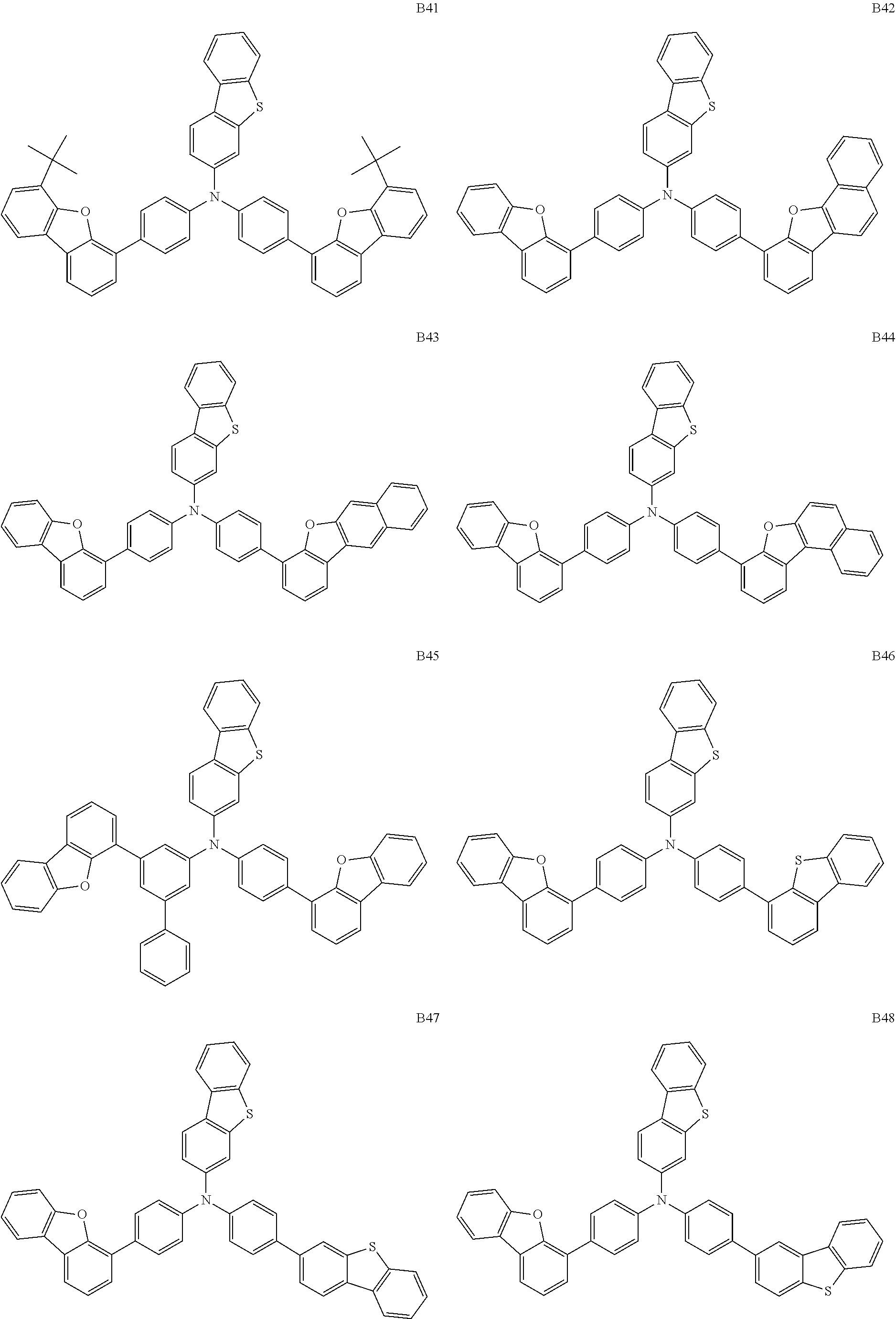

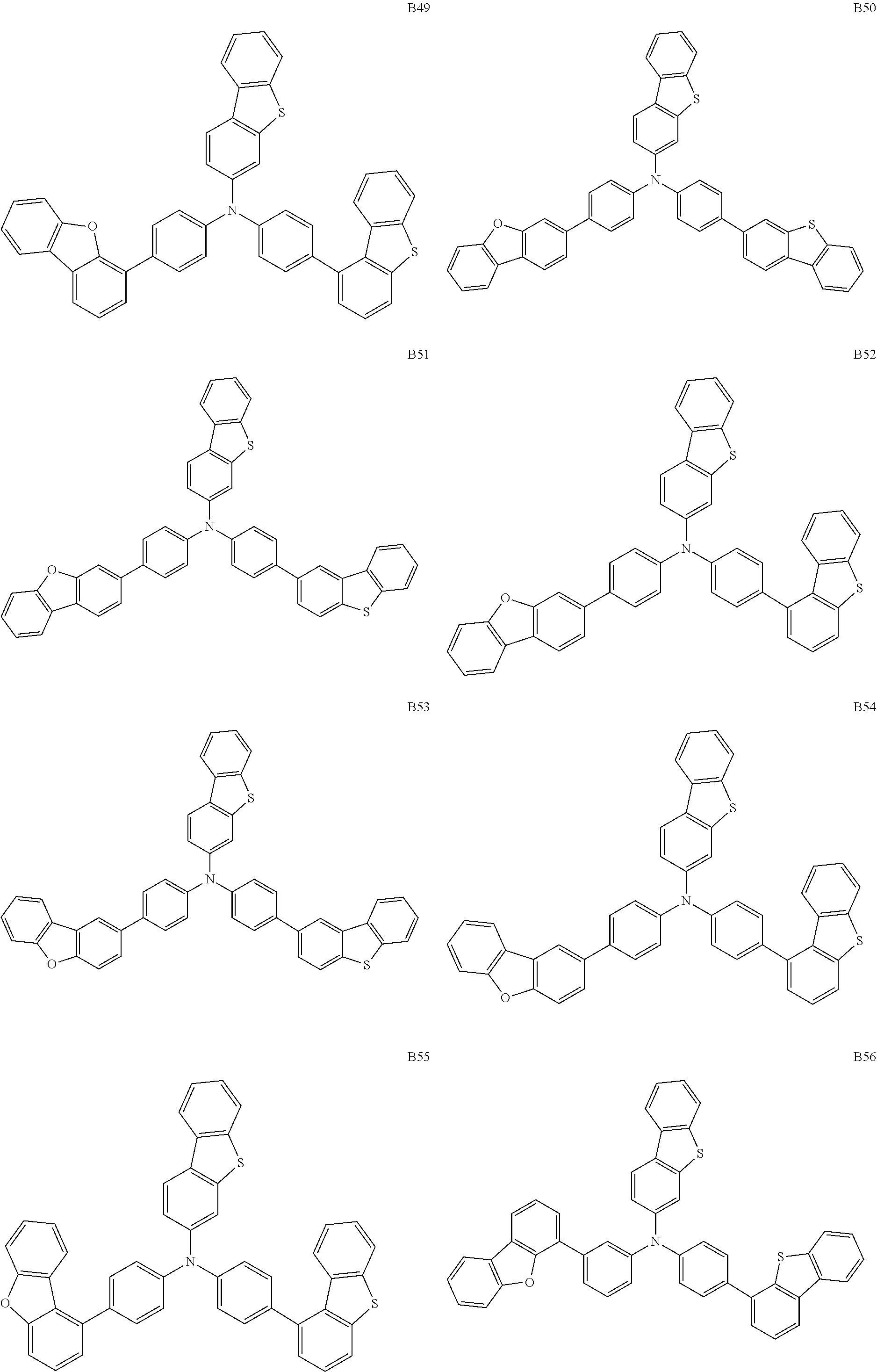

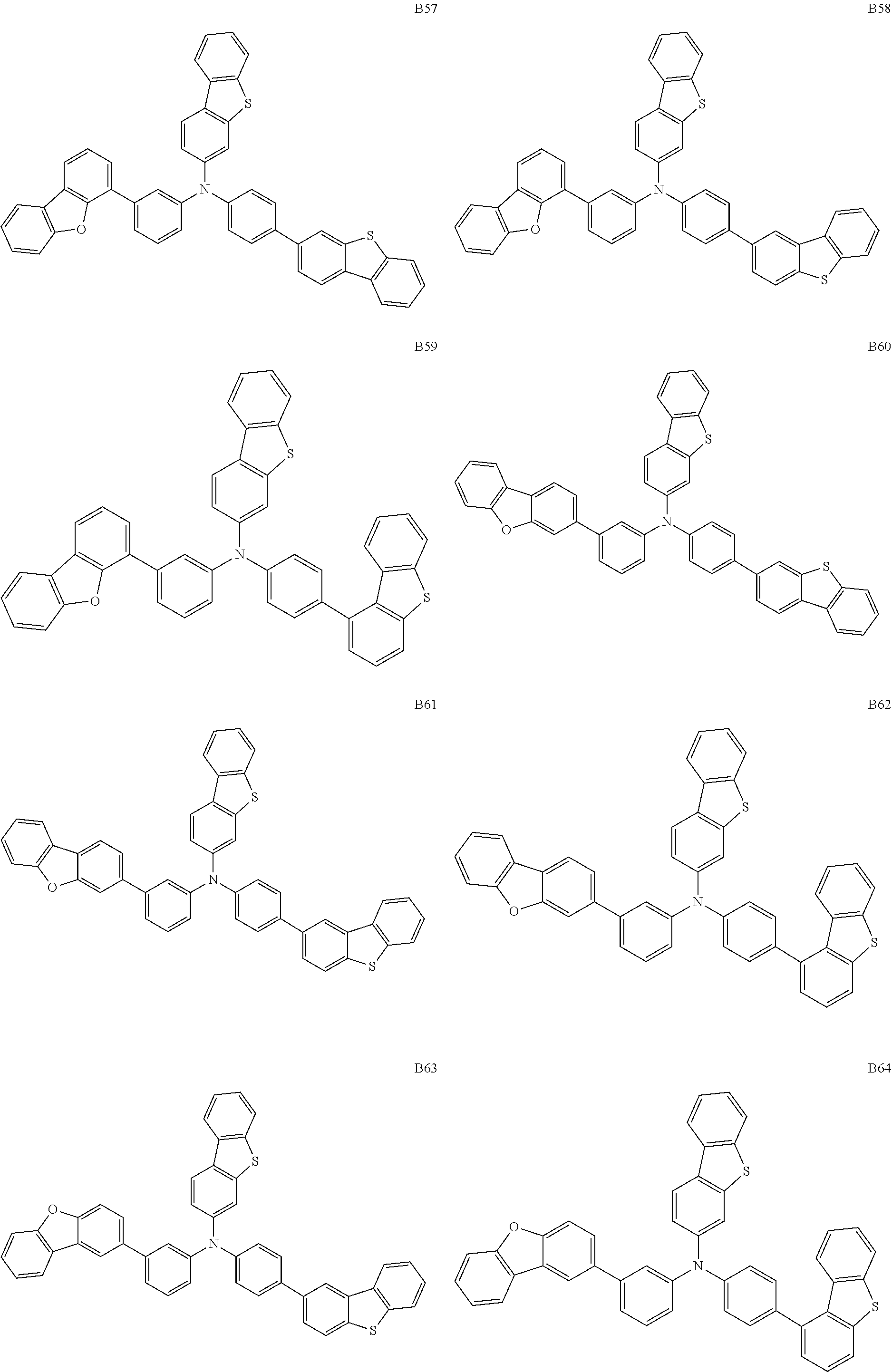

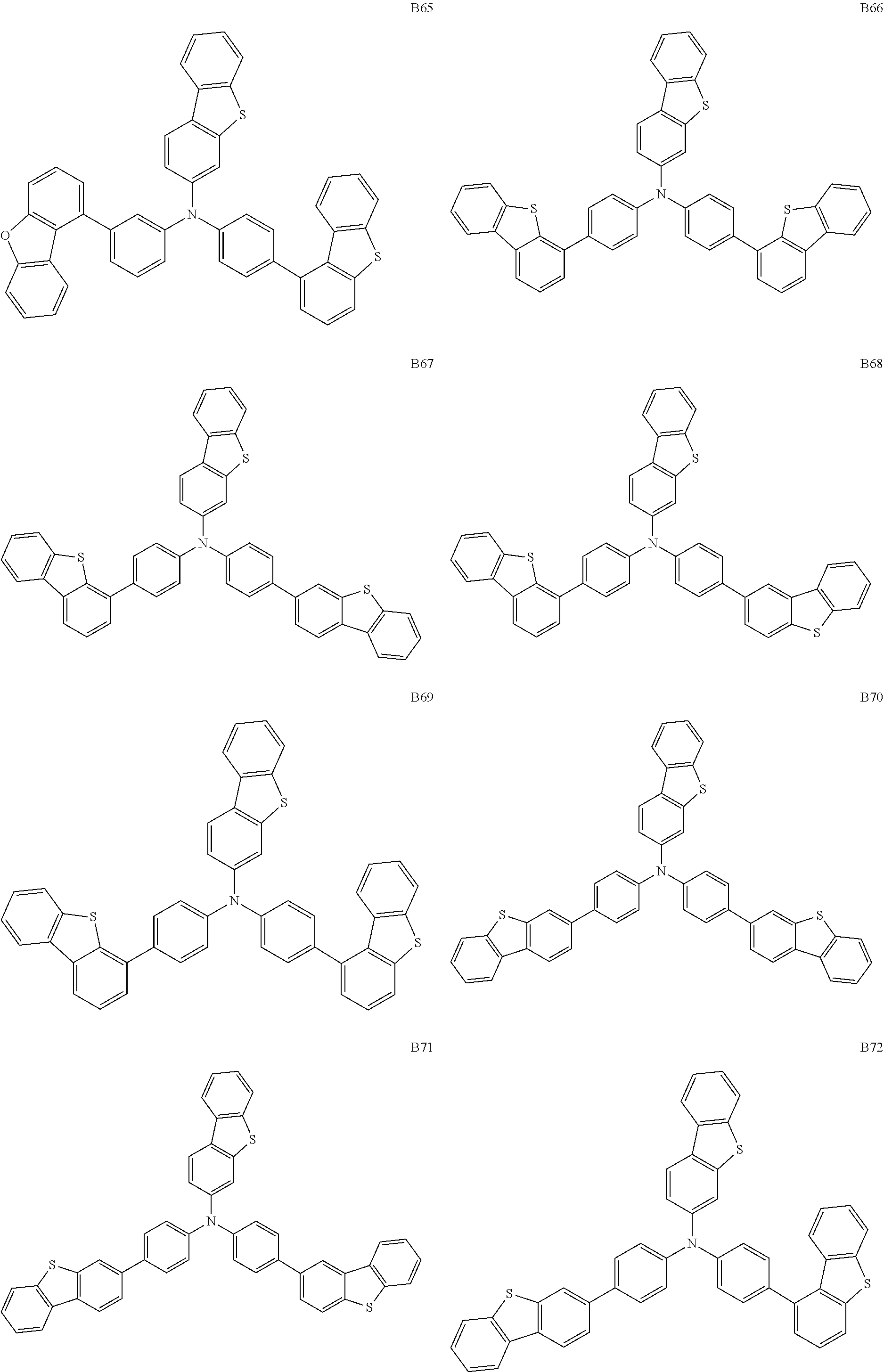

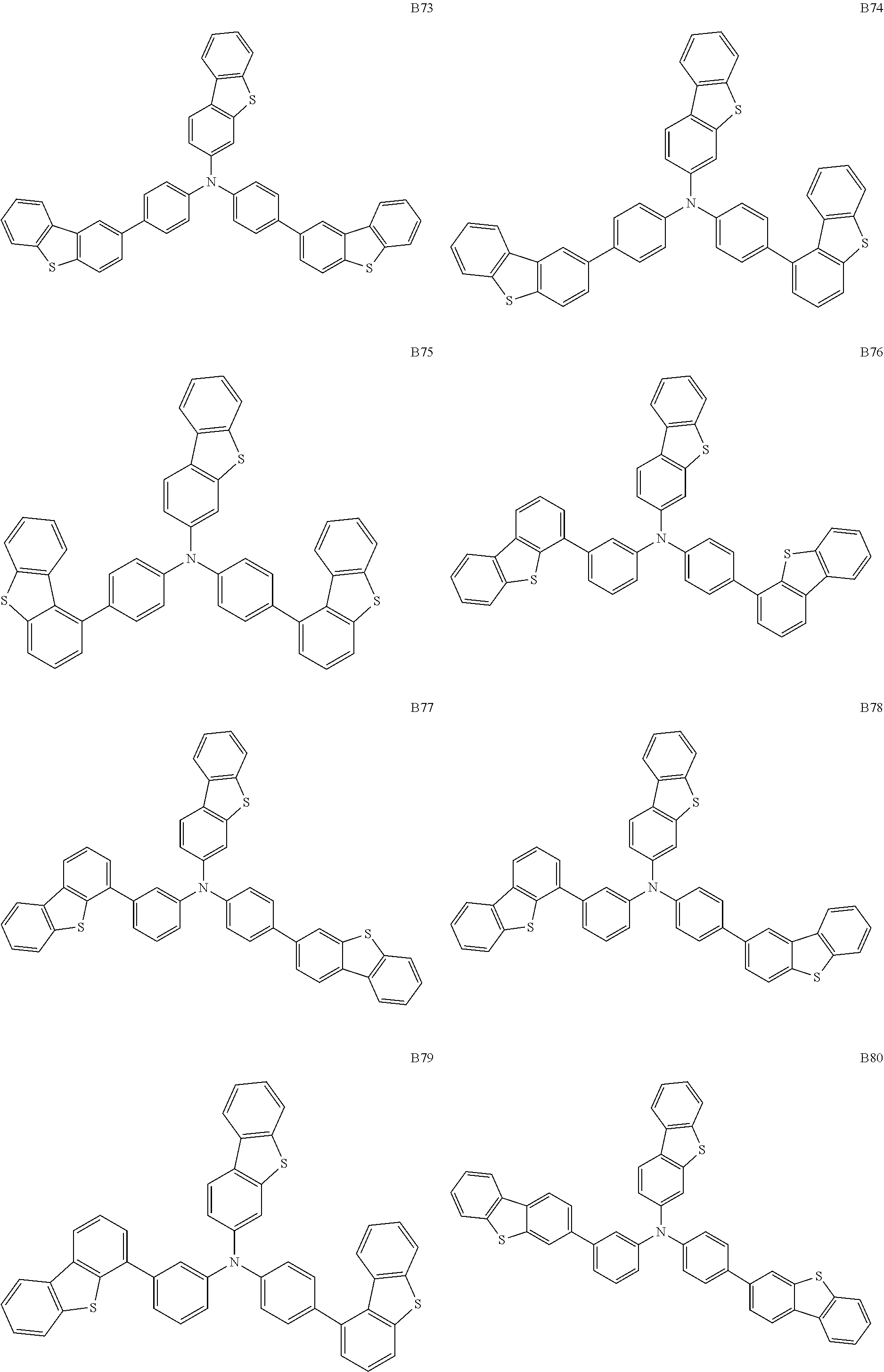

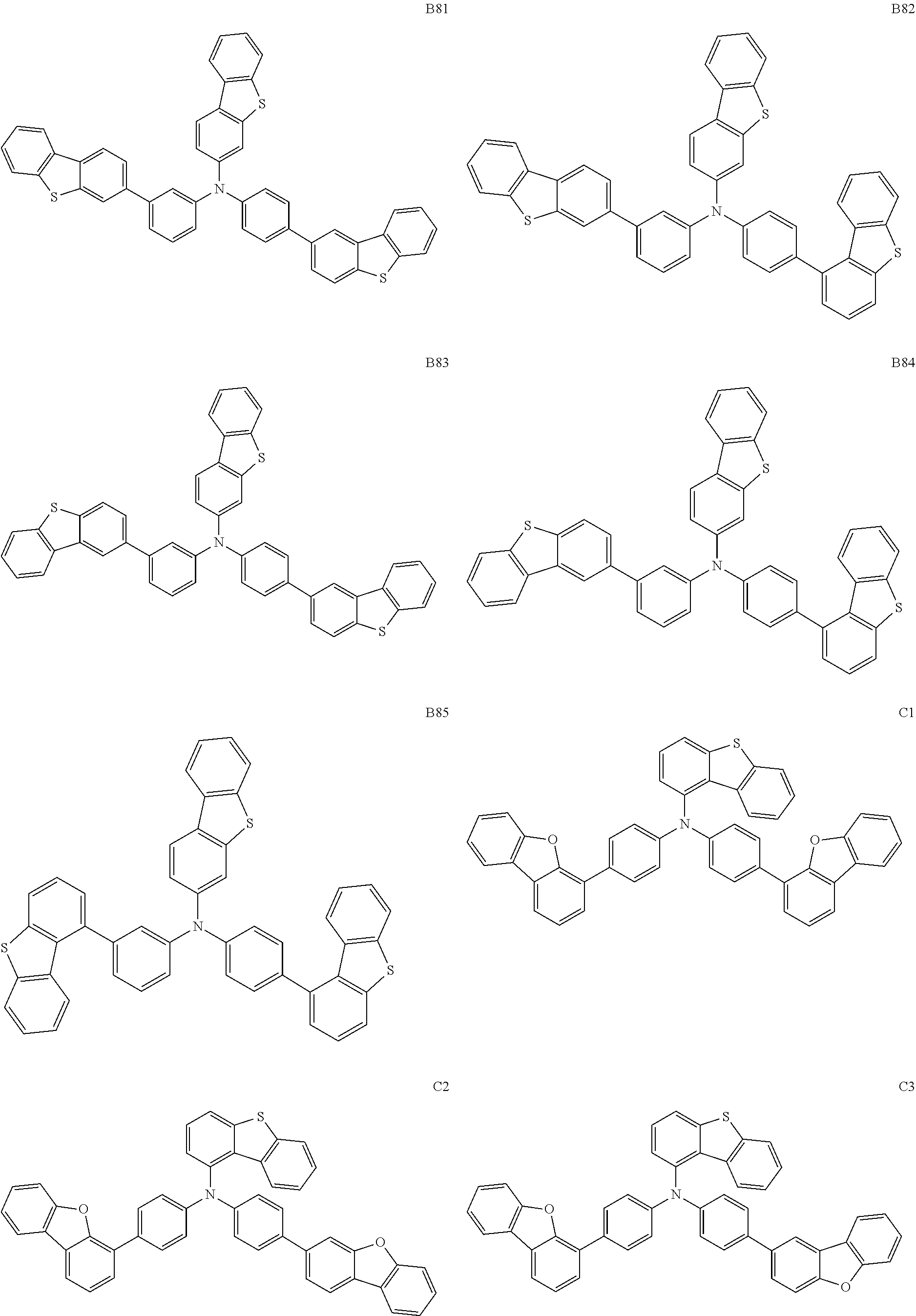

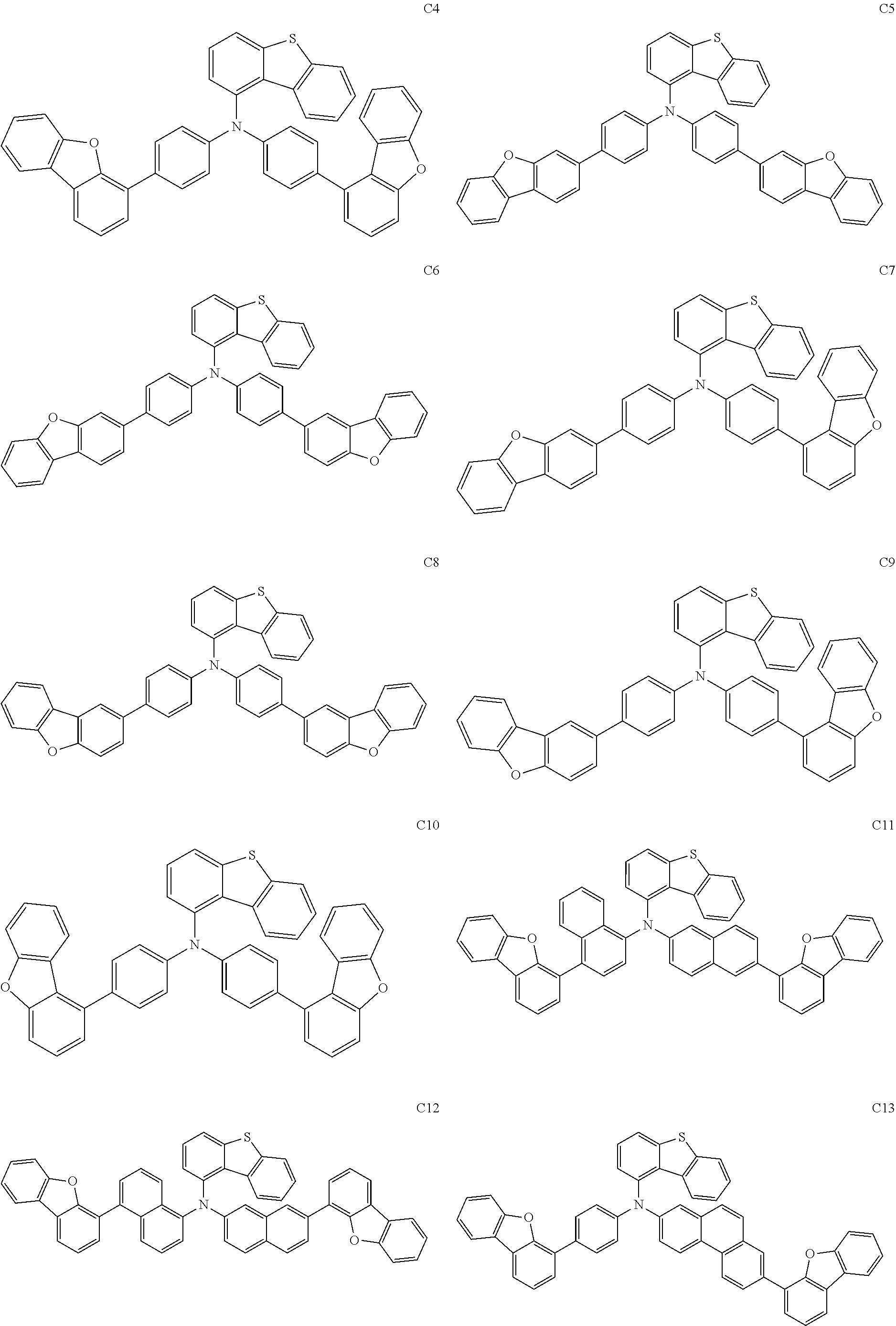

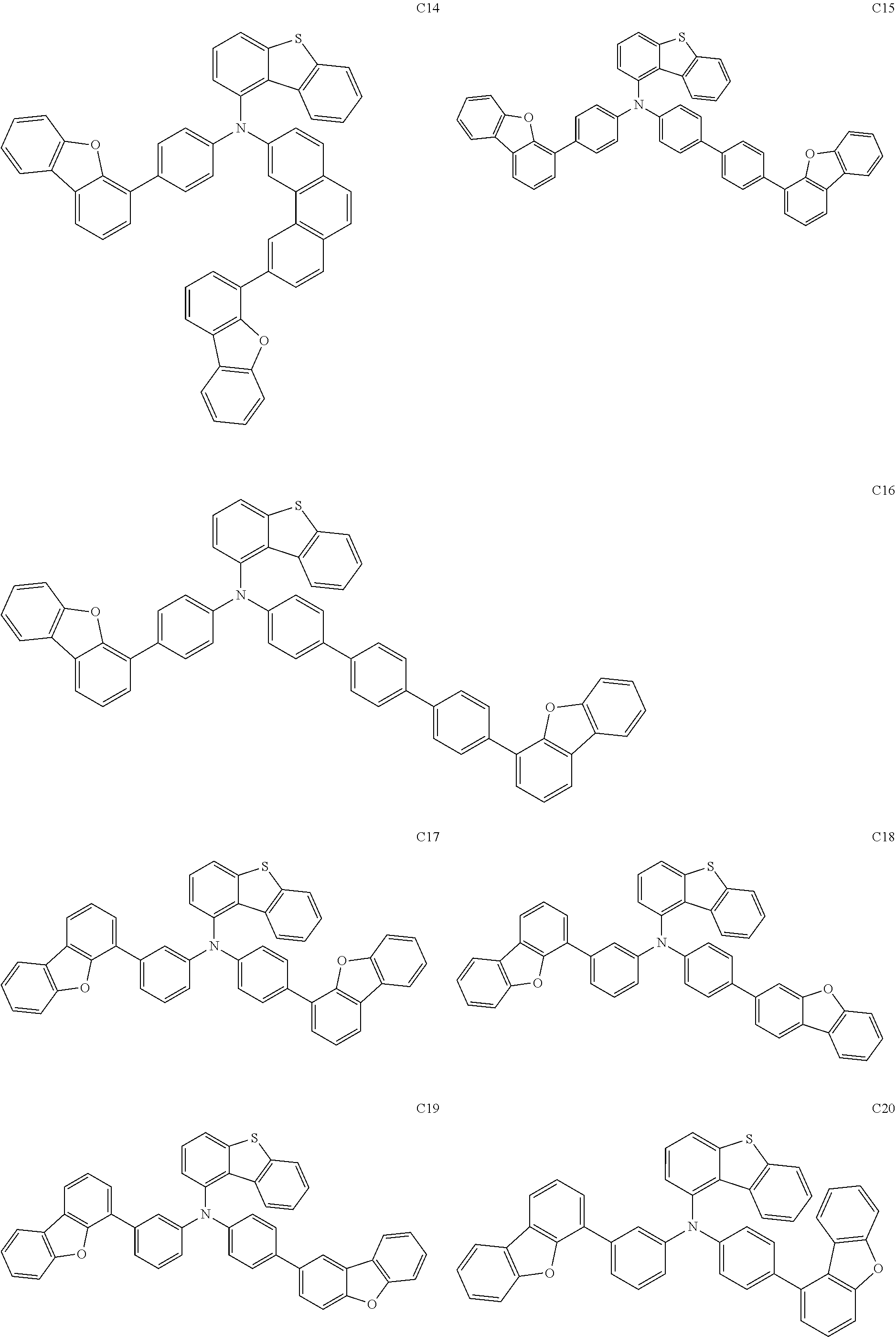

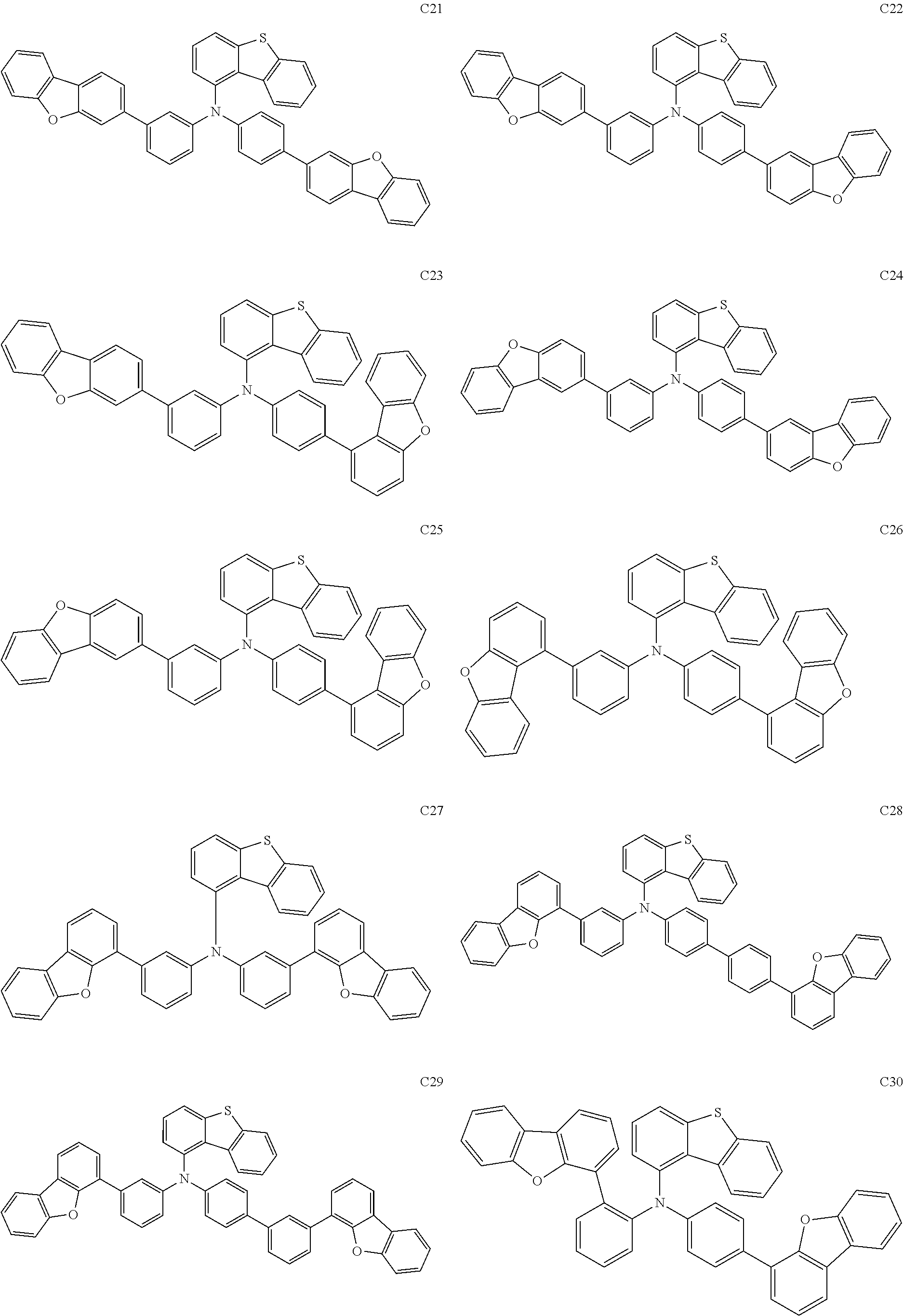

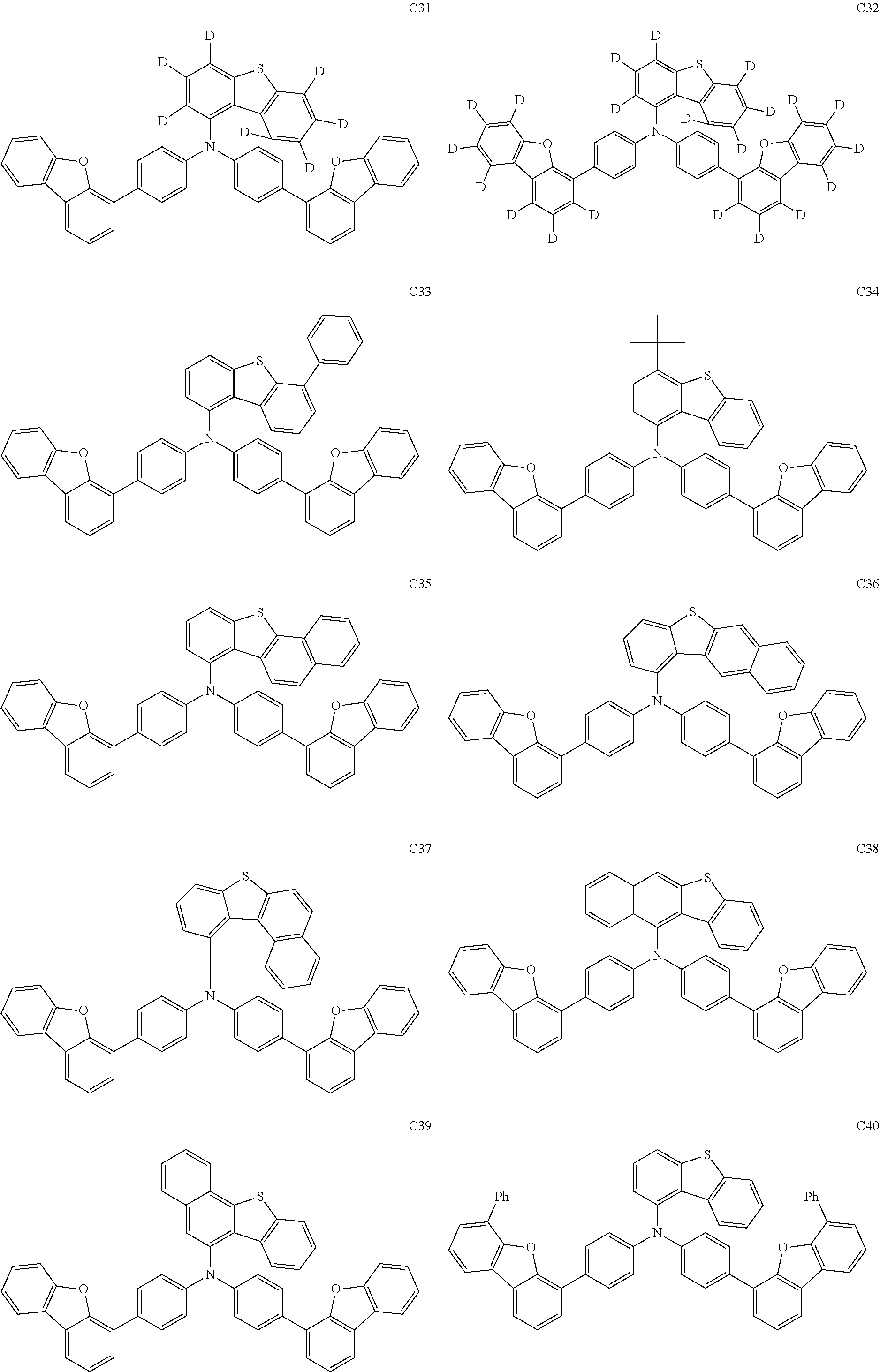

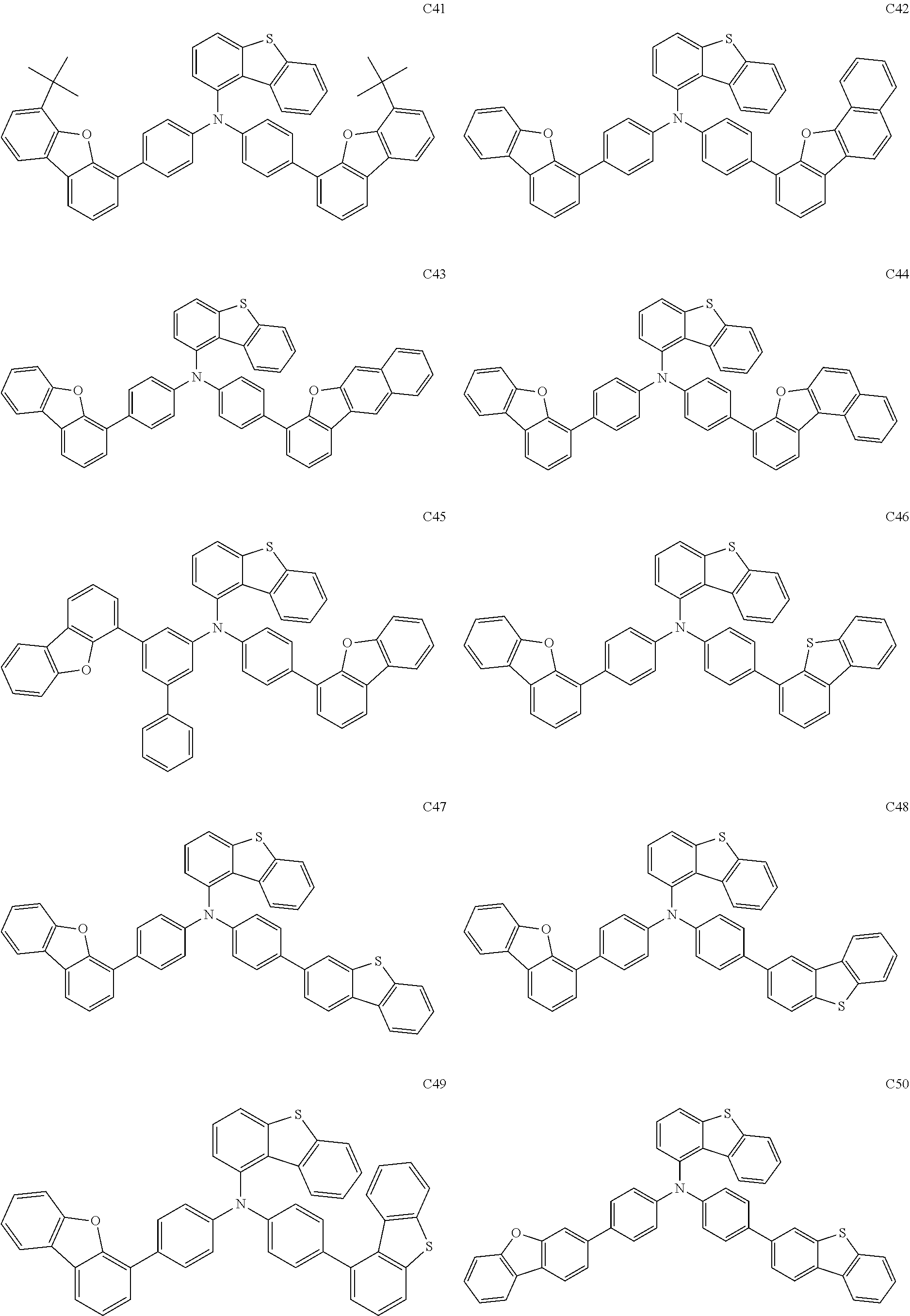

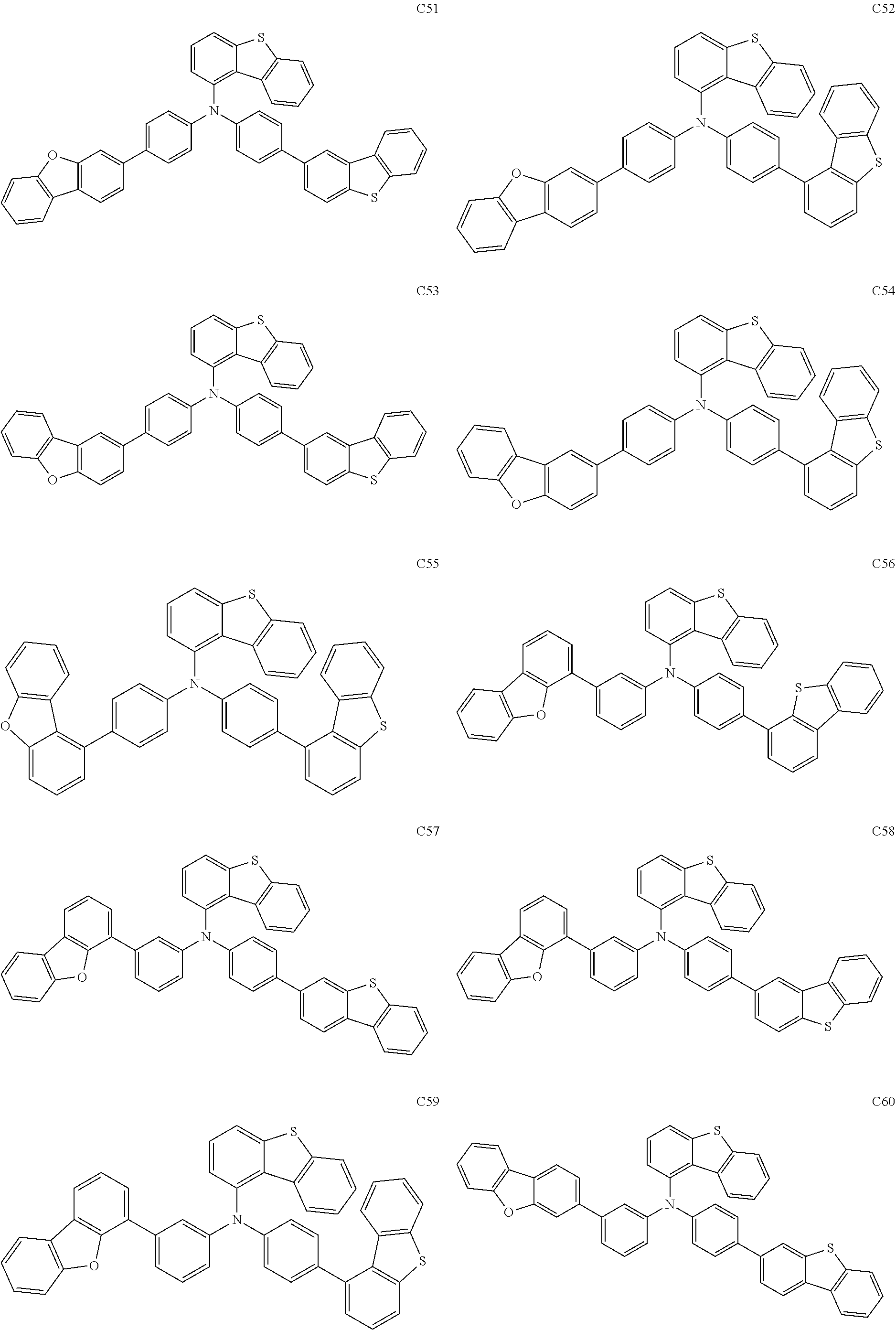

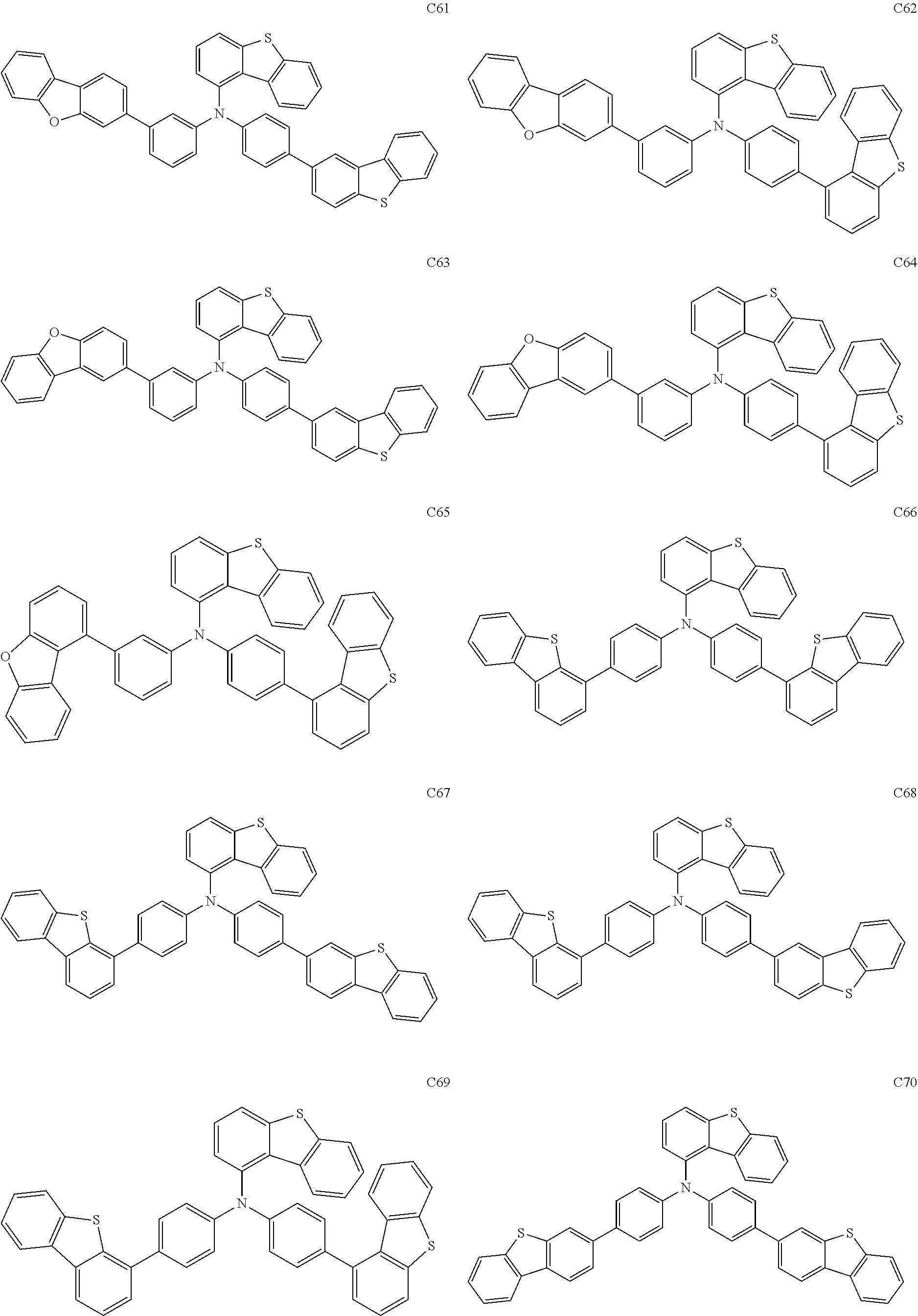

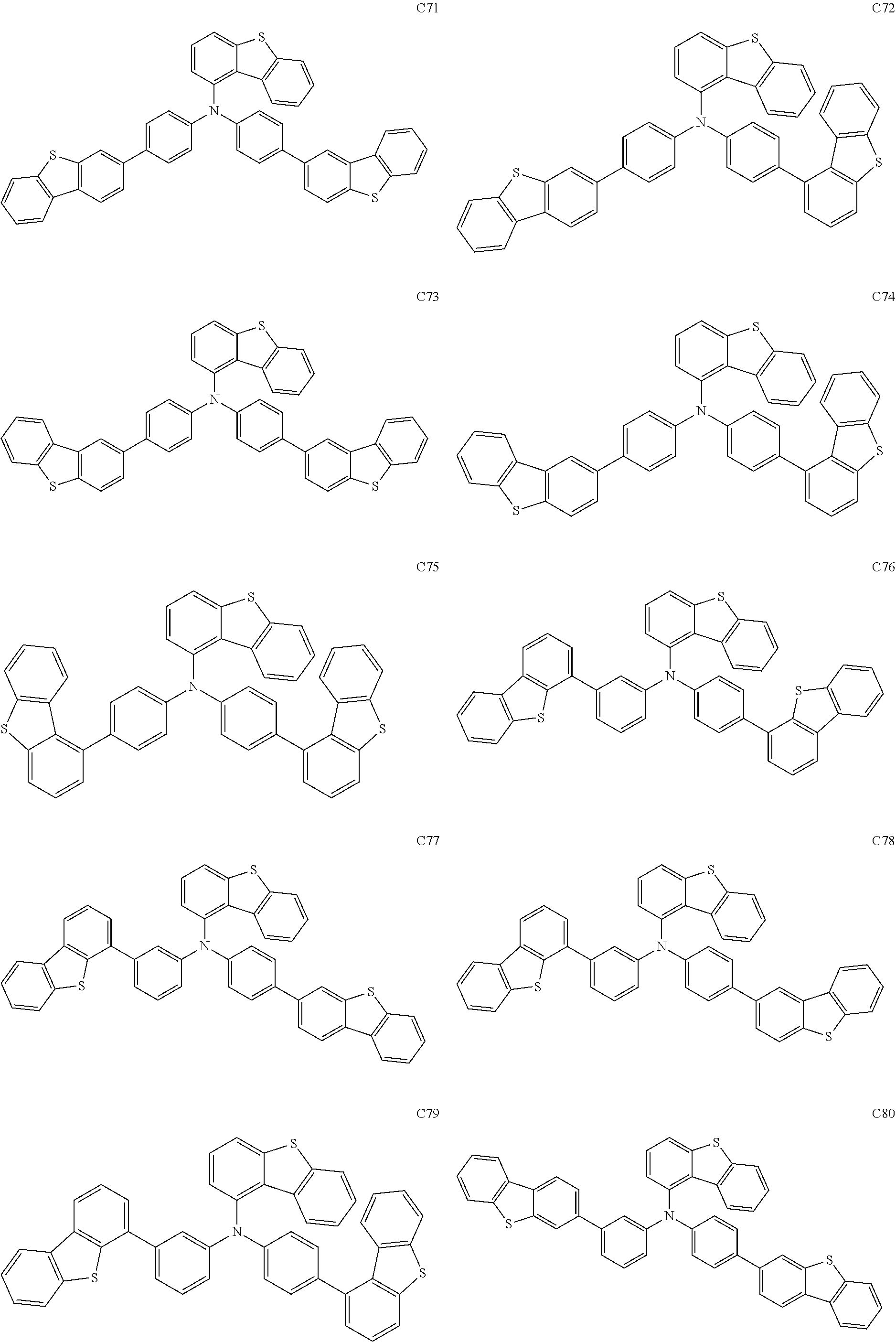

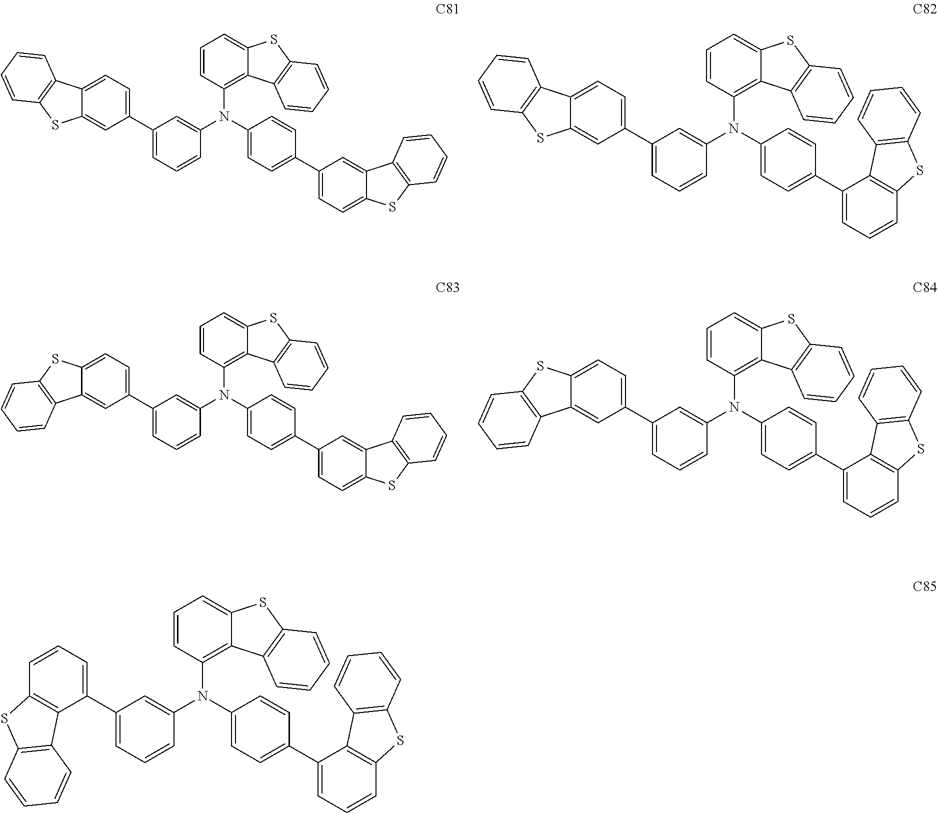

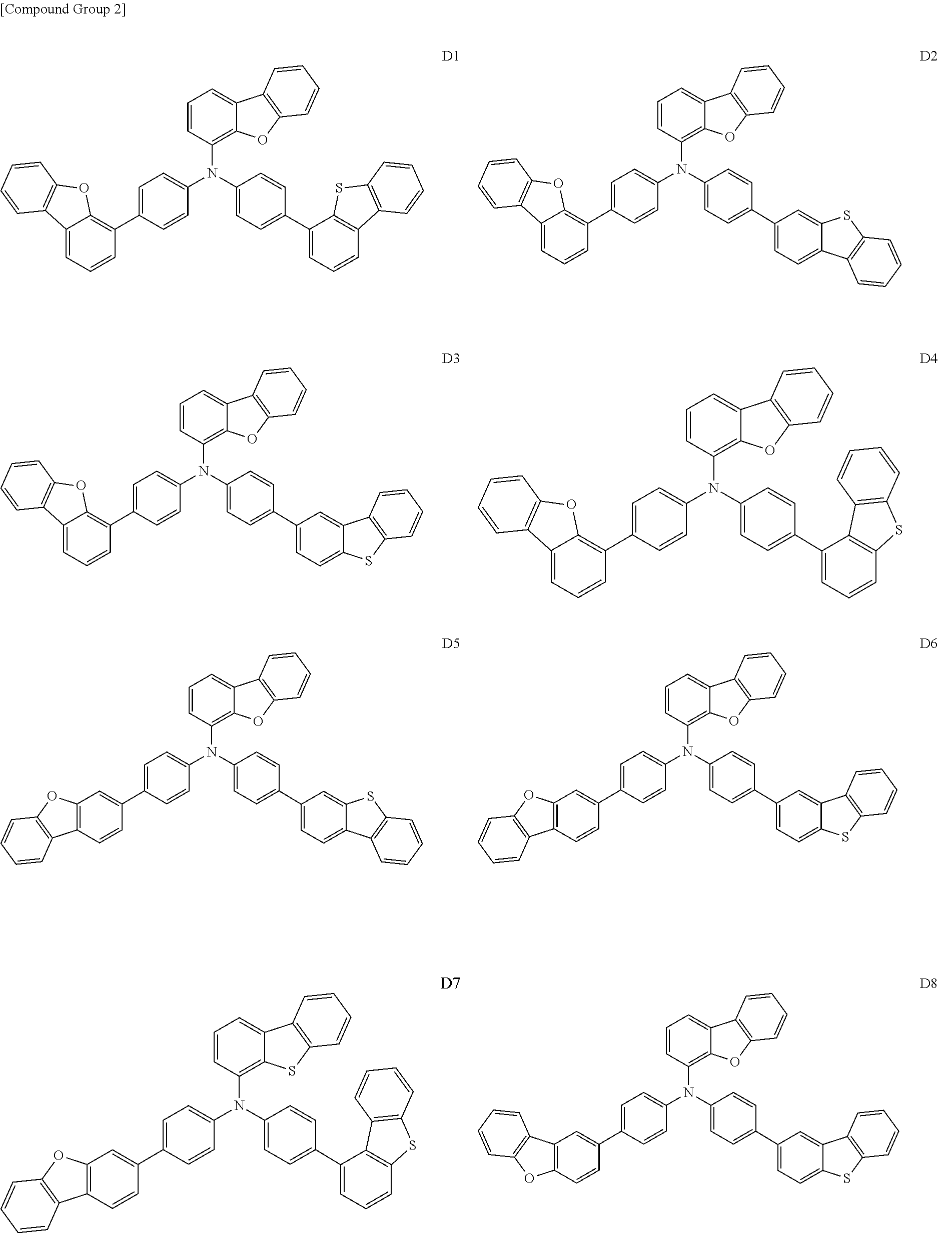

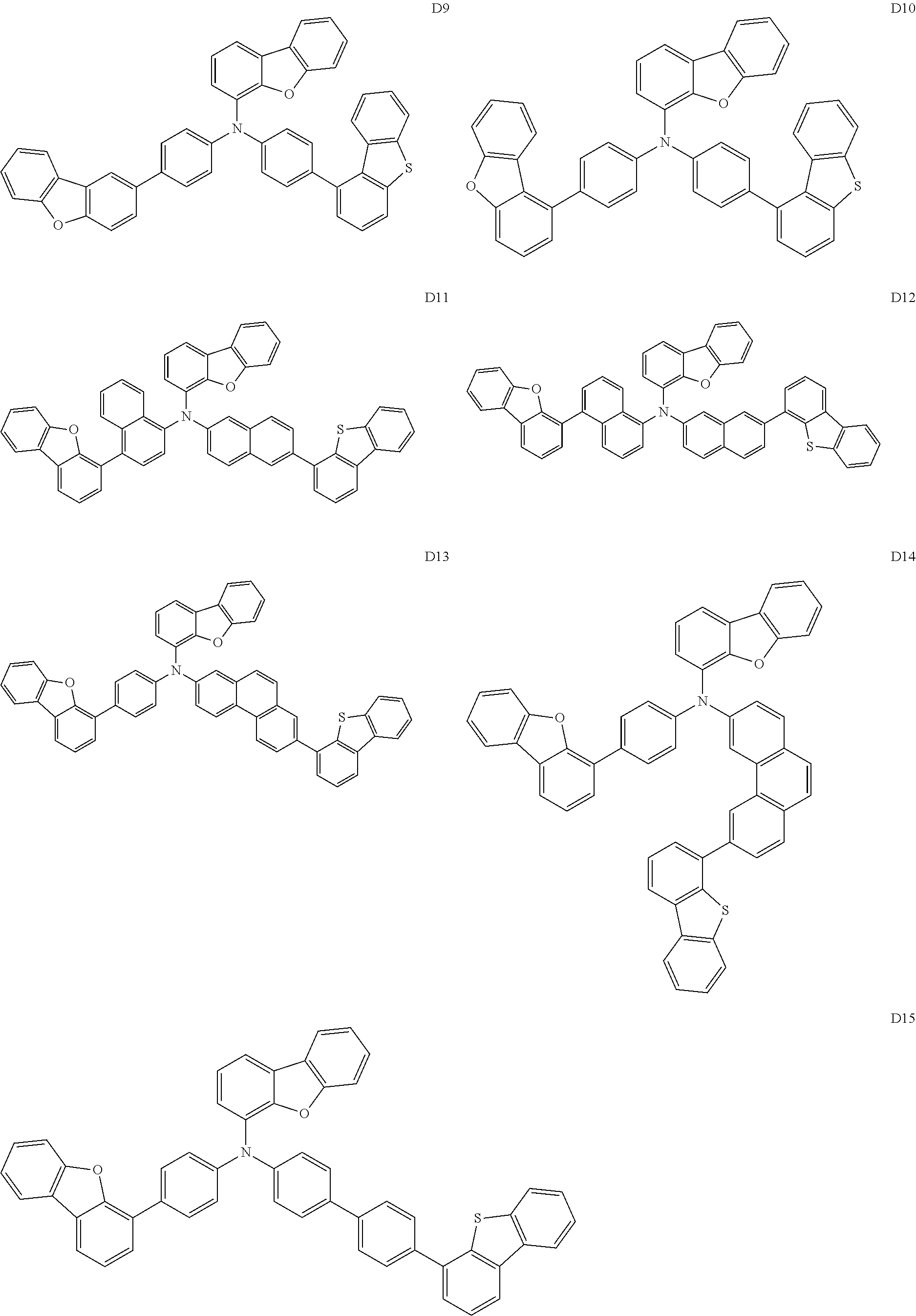

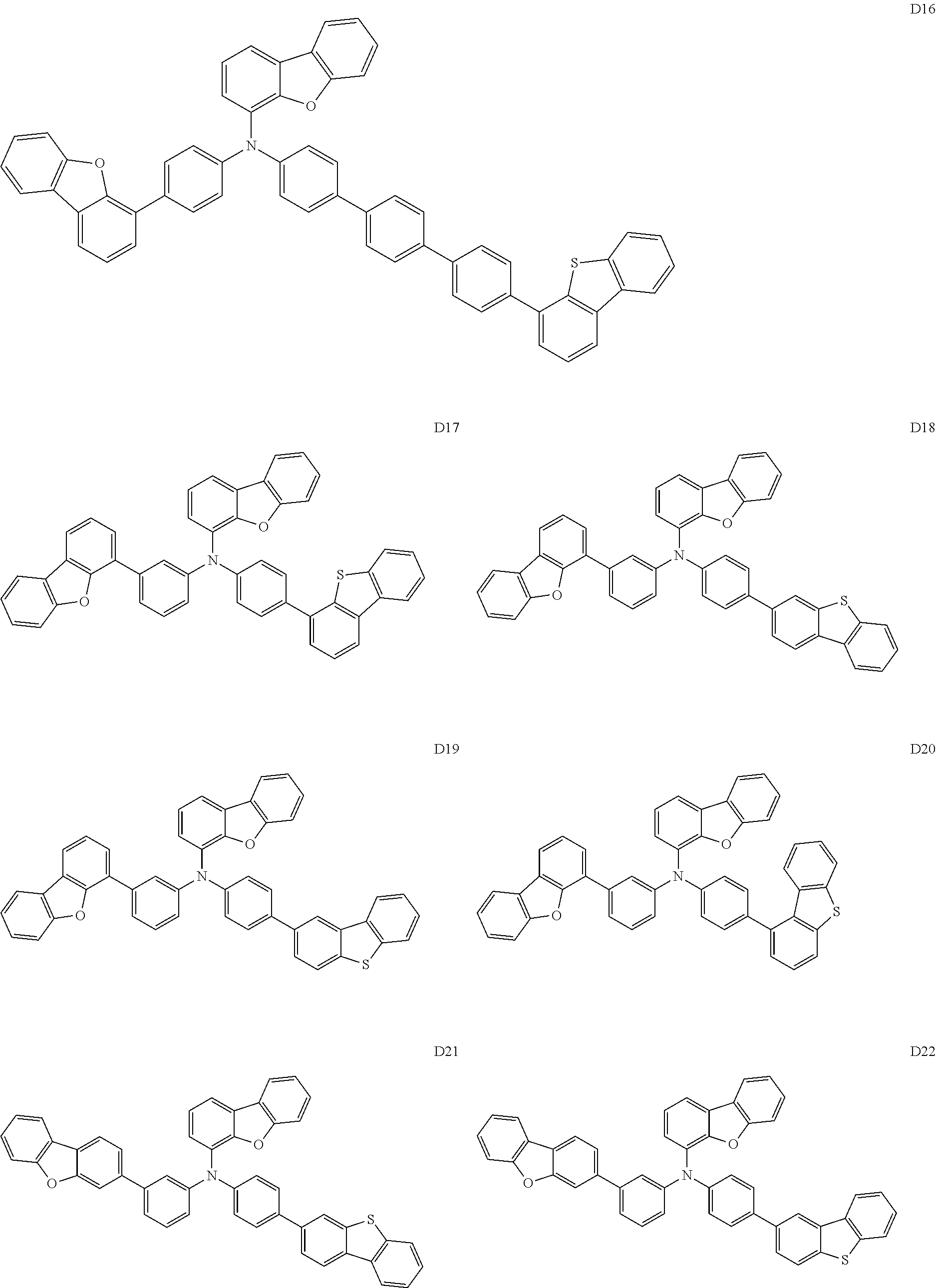

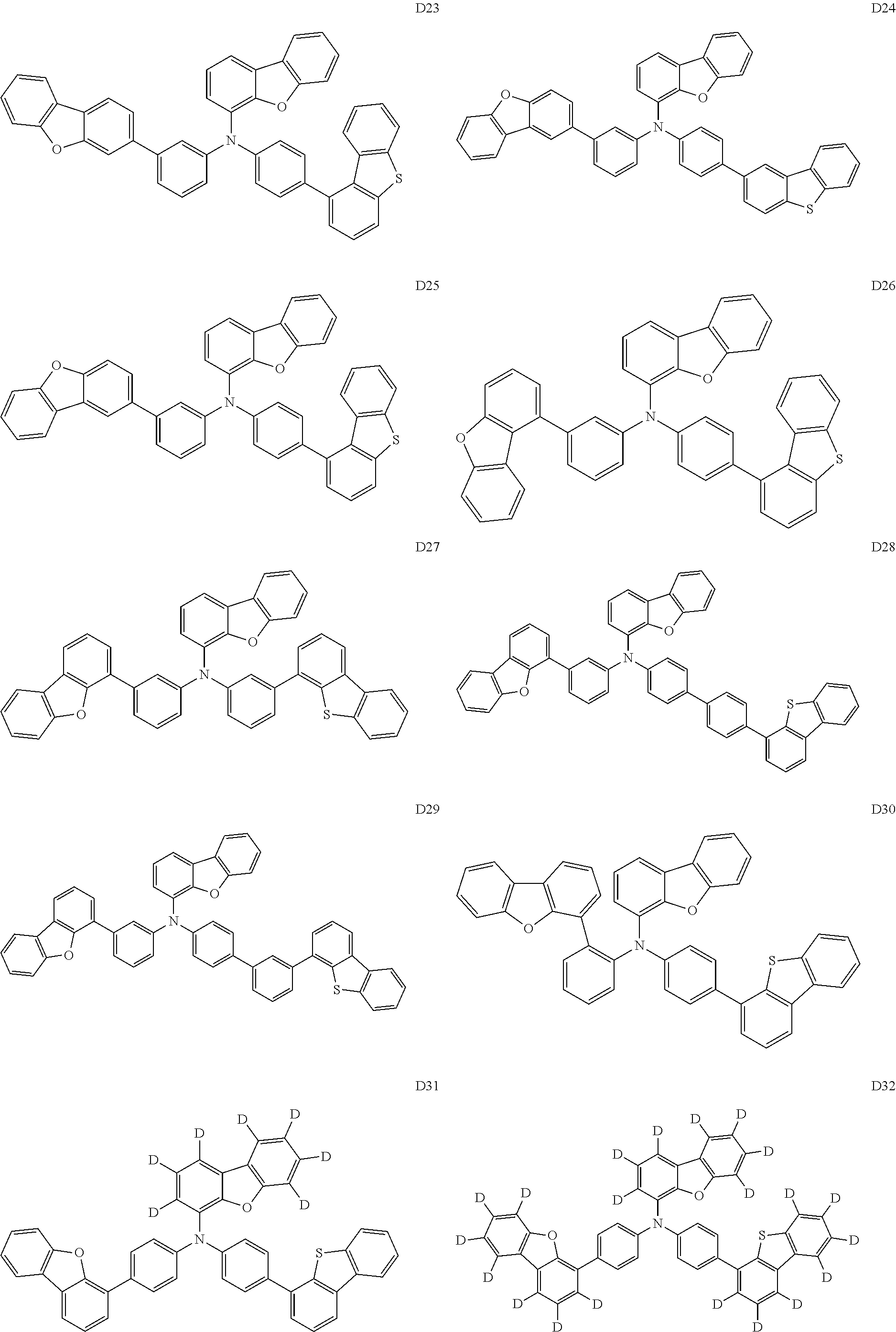

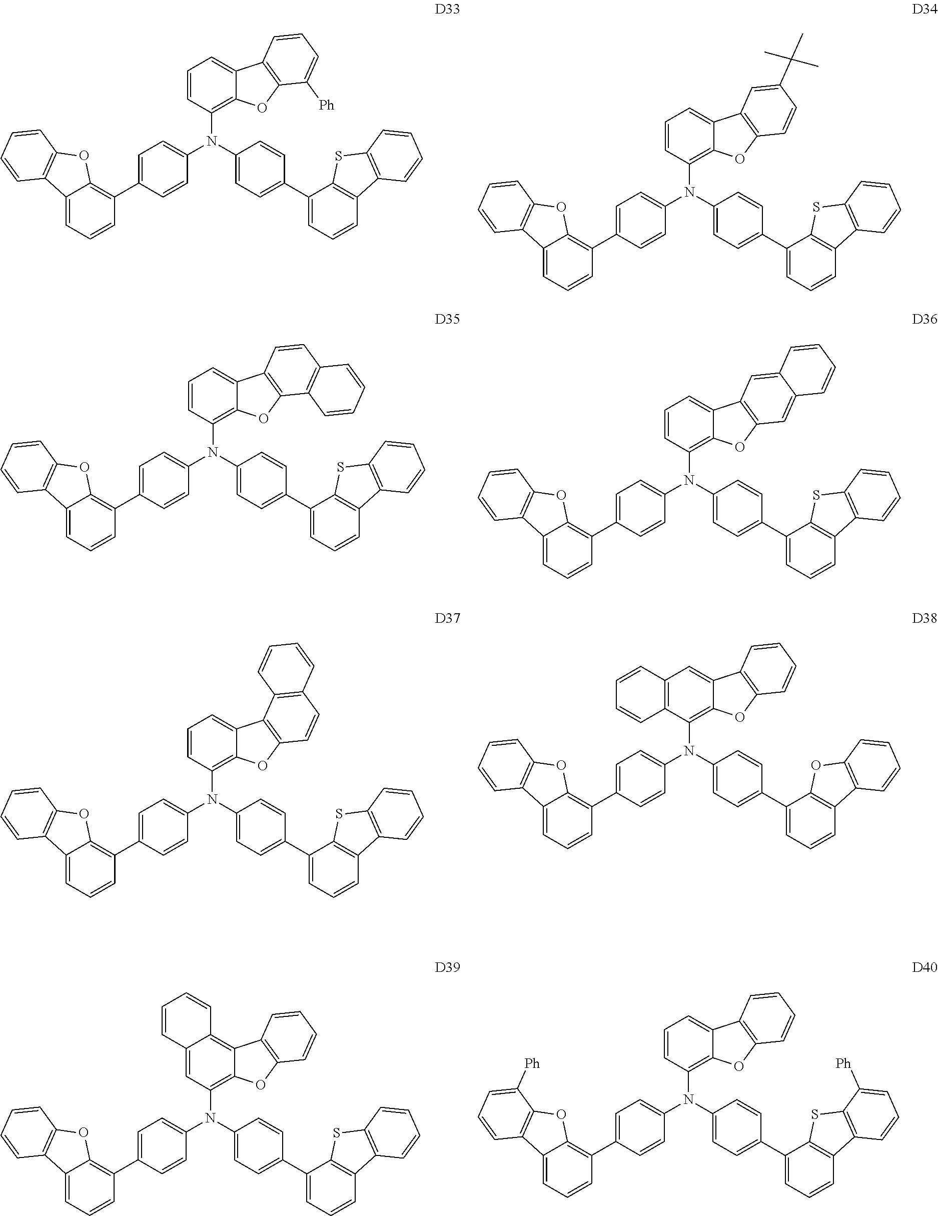

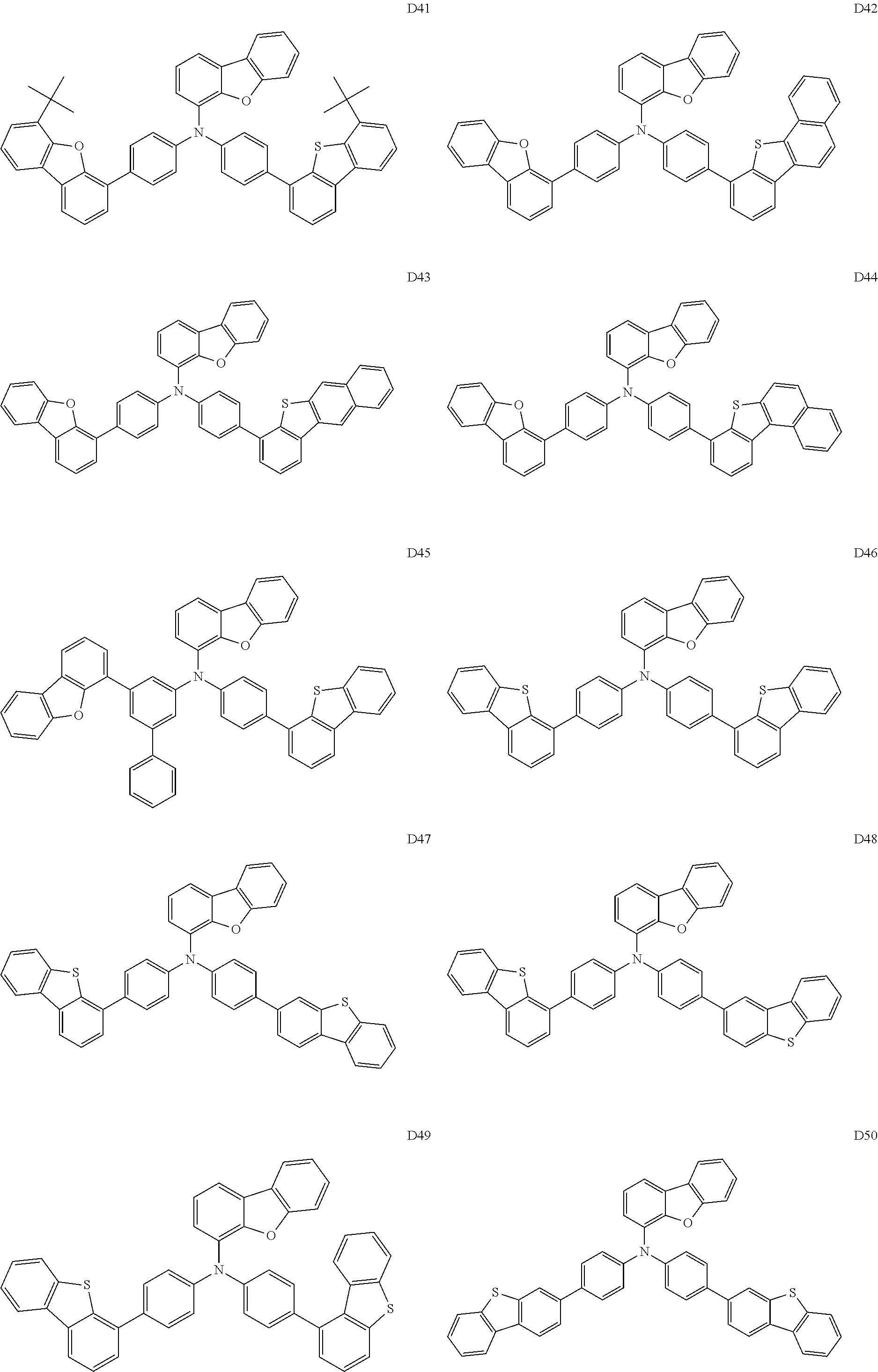

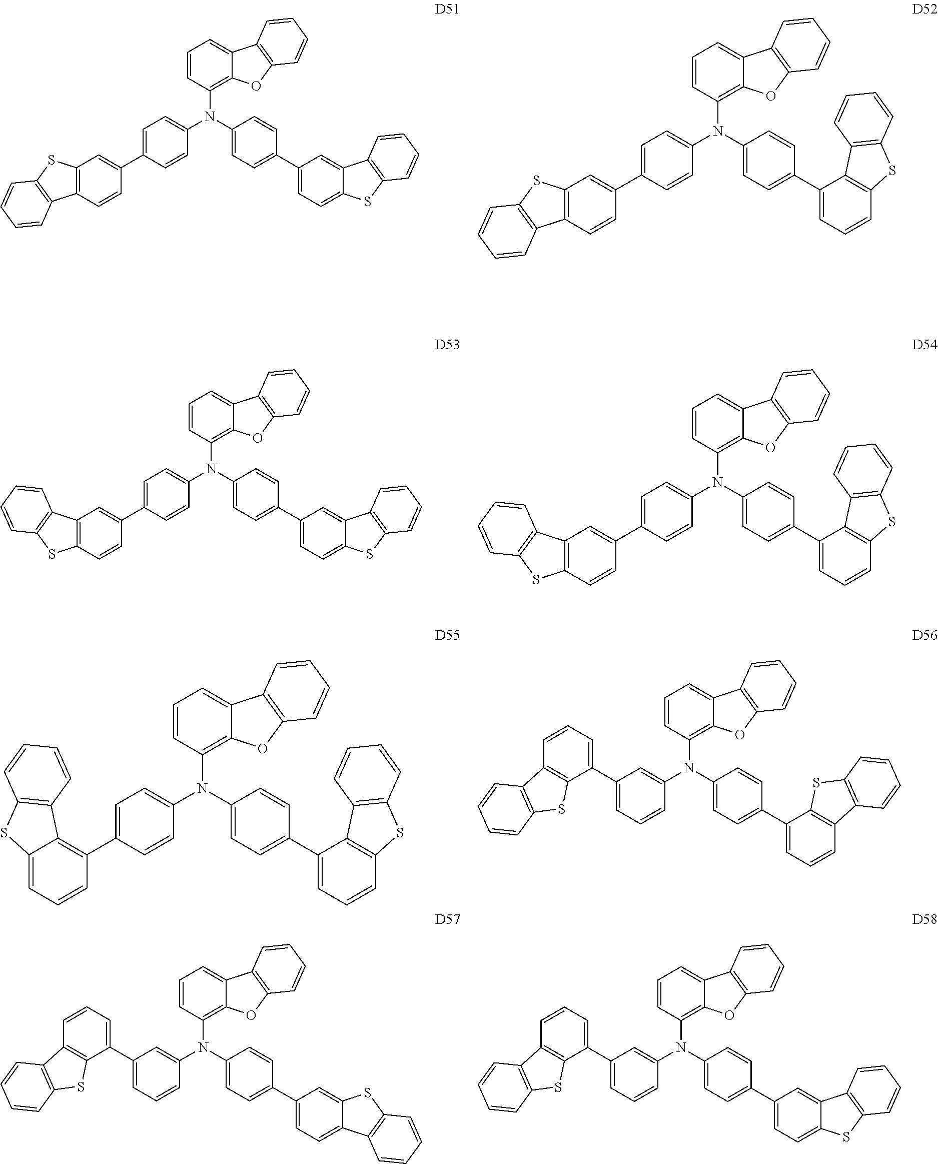

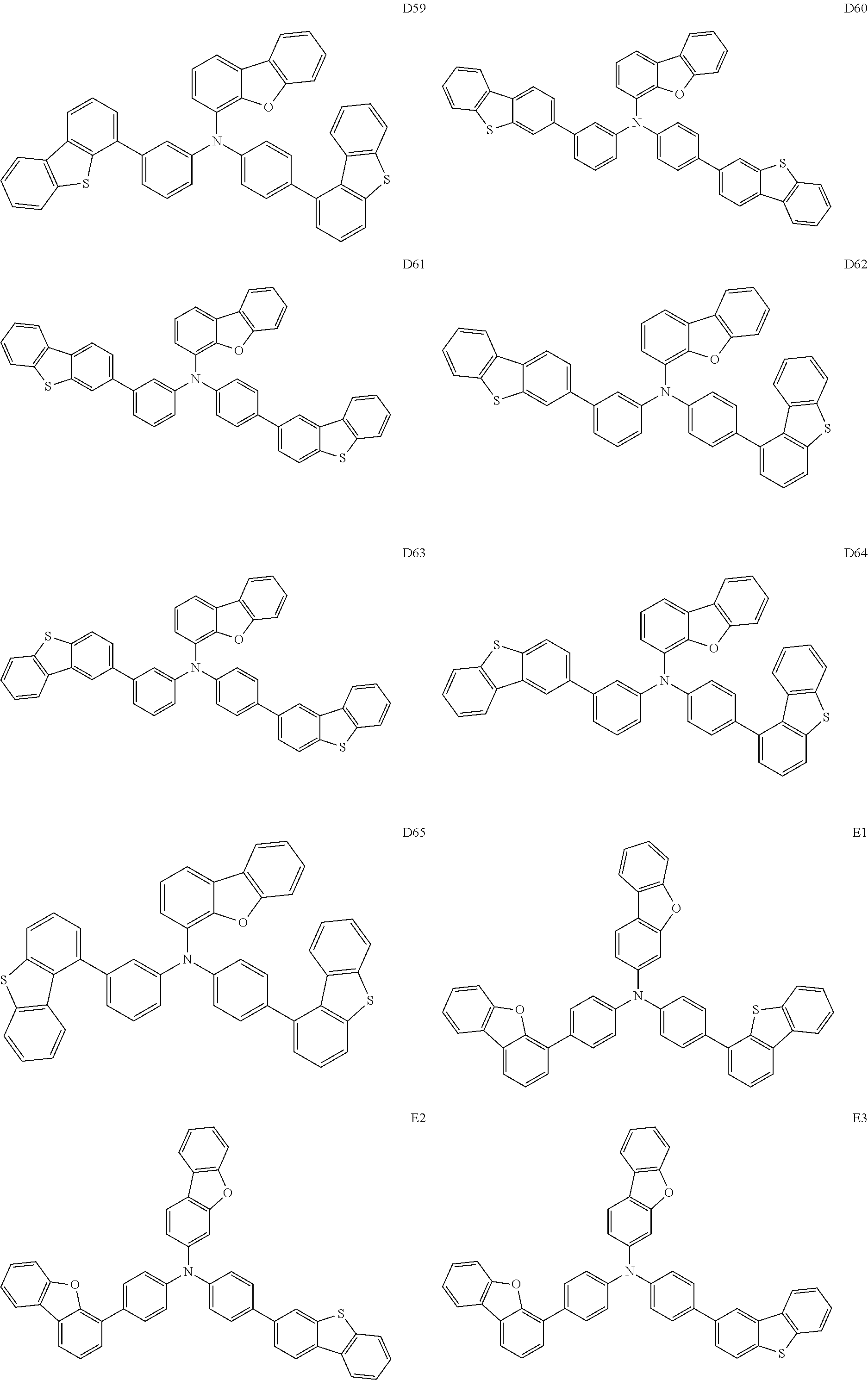

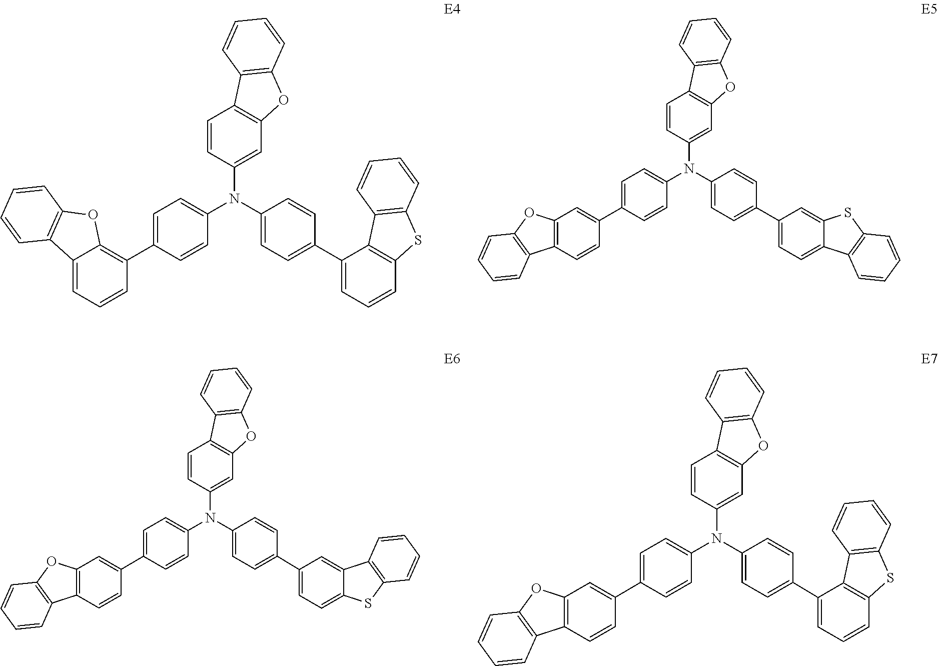 [SMILES: c1ccc2c(c1)oc1cc(N(c3ccc(-c4cccc5c4oc4ccccc45)cc3)c3ccc(-c4cccc5sc6ccccc6c45)cc3)ccc12 ; c1ccc2c(c1)oc1cc(-c3ccc(N(c4ccc(-c5ccc6c(c5)sc5ccccc56)cc4)c4ccc5c(c4)oc4ccccc45)cc3)ccc12 ; c1ccc2c(c1)oc1cc(-c3ccc(N(c4ccc(-c5ccc6sc7ccccc7c6c5)cc4)c4ccc5c(c4)oc4ccccc45)cc3)ccc12 ; c1ccc2c(c1)oc1cc(-c3ccc(N(c4ccc(-c5cccc6sc7ccccc7c56)cc4)c4ccc5c(c4)oc4ccccc45)cc3)ccc12]

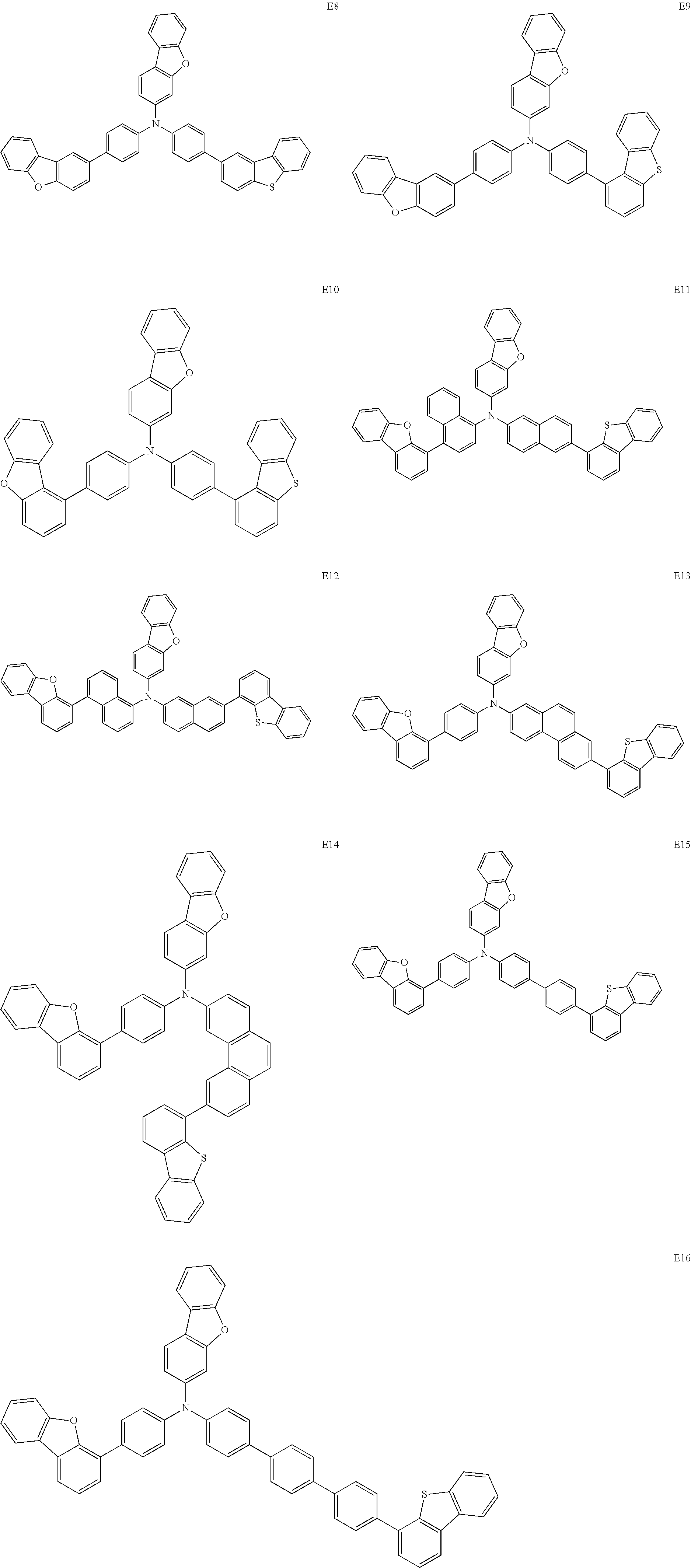

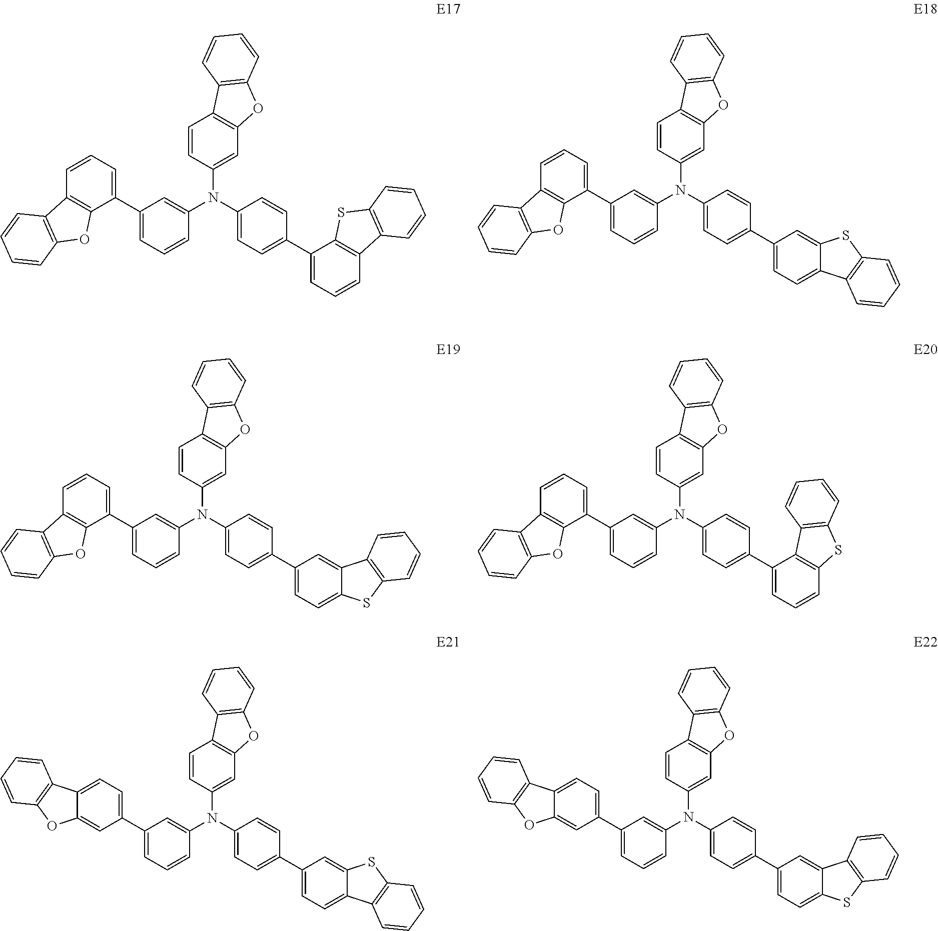

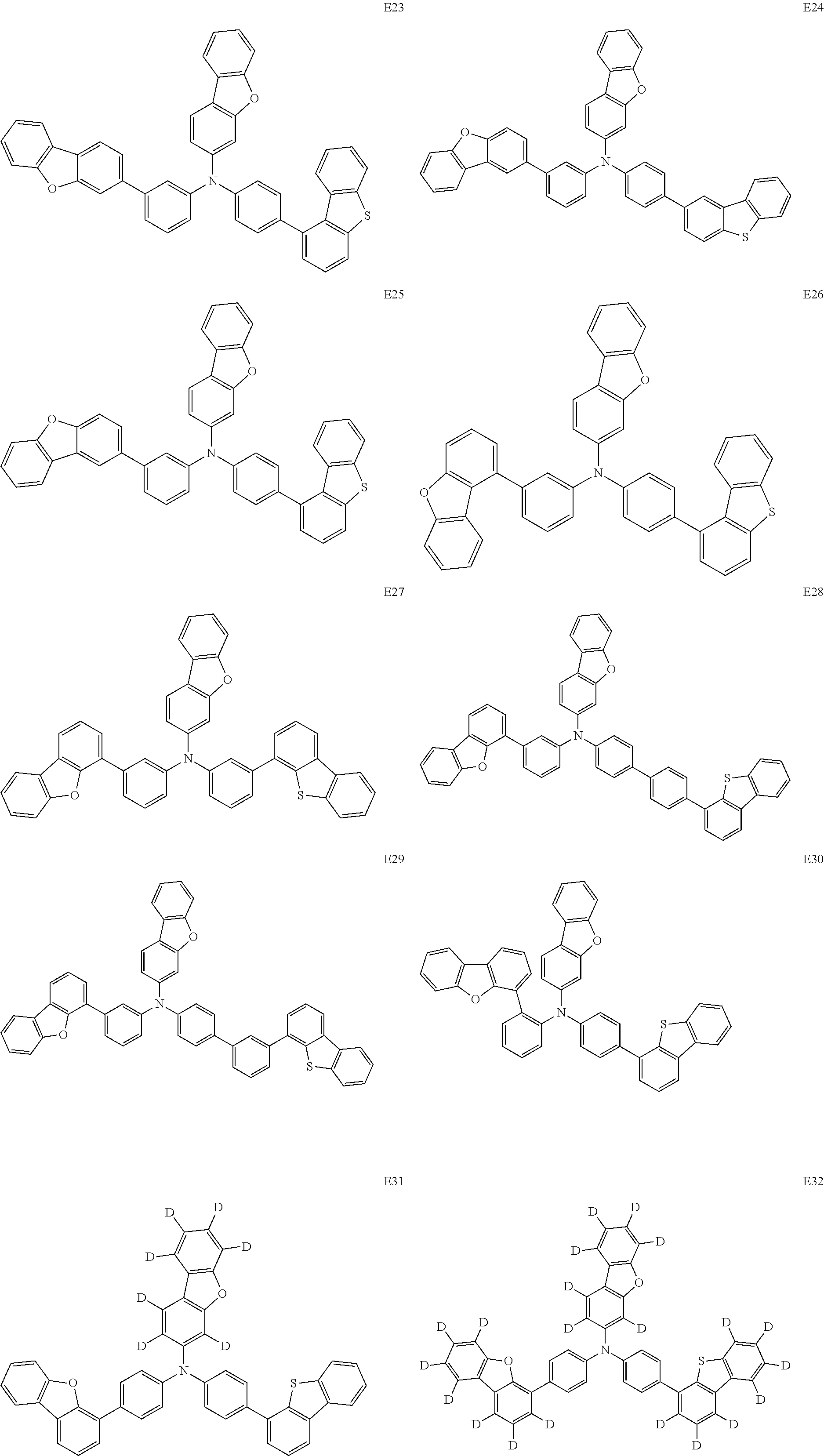

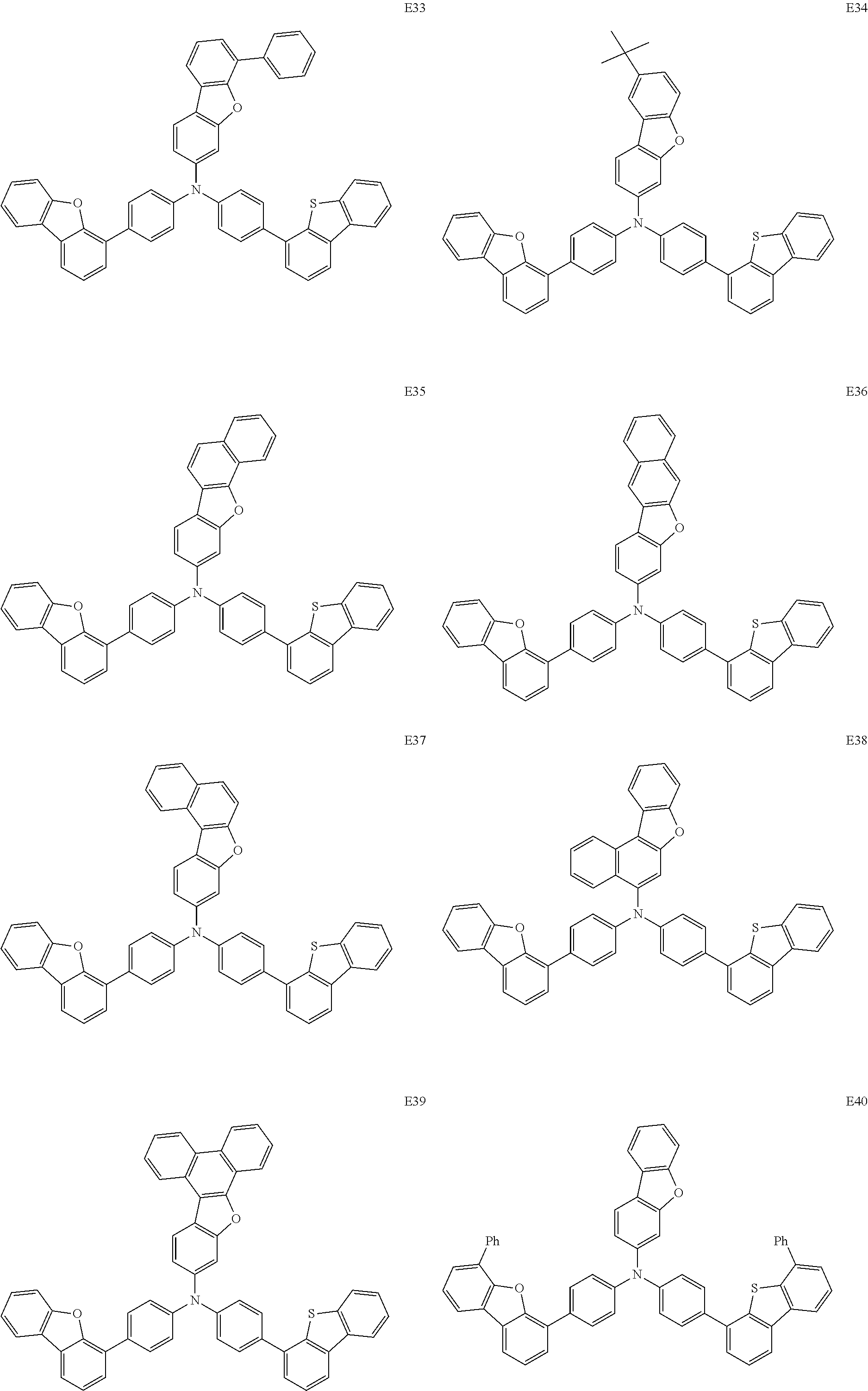

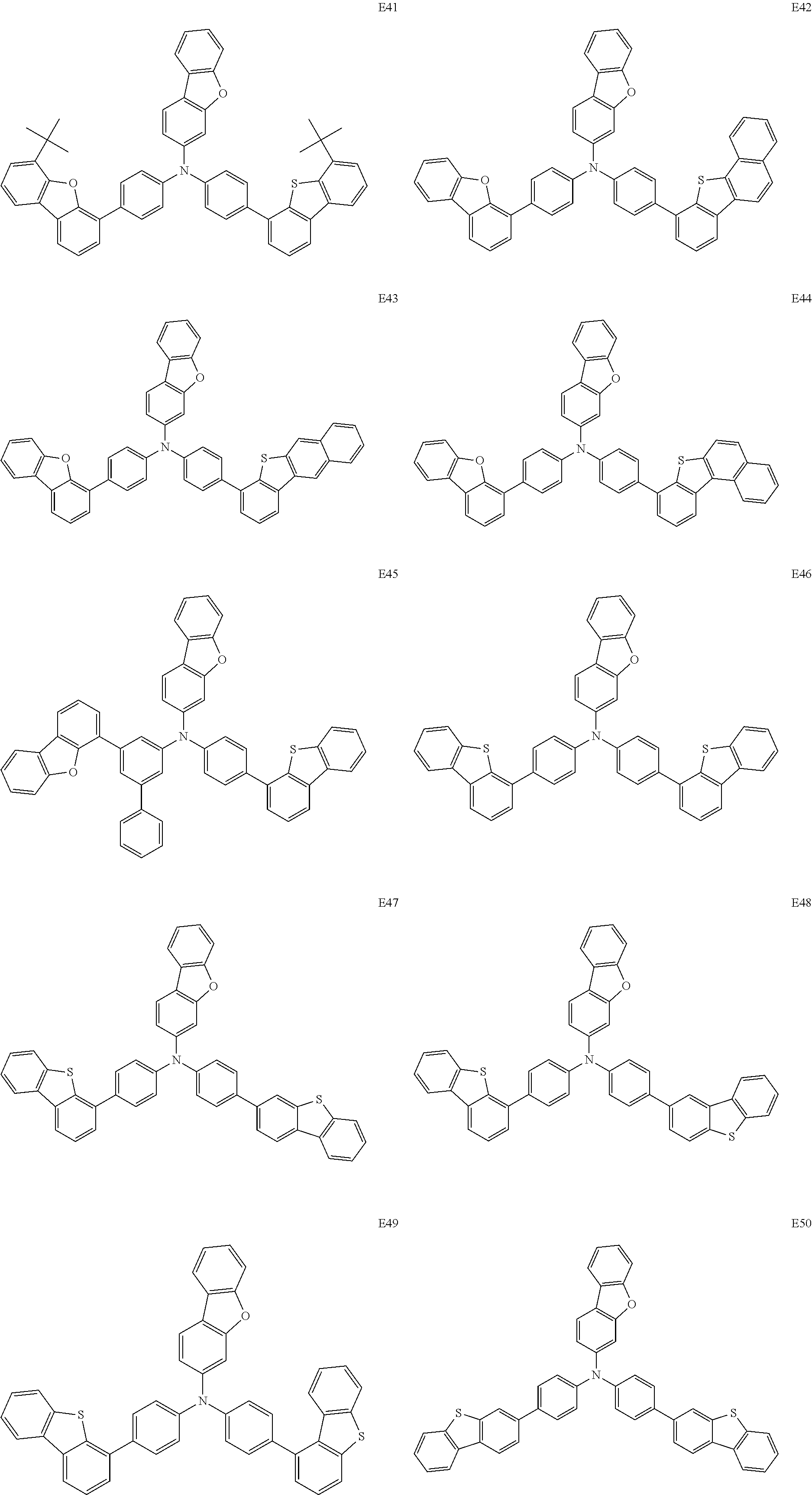

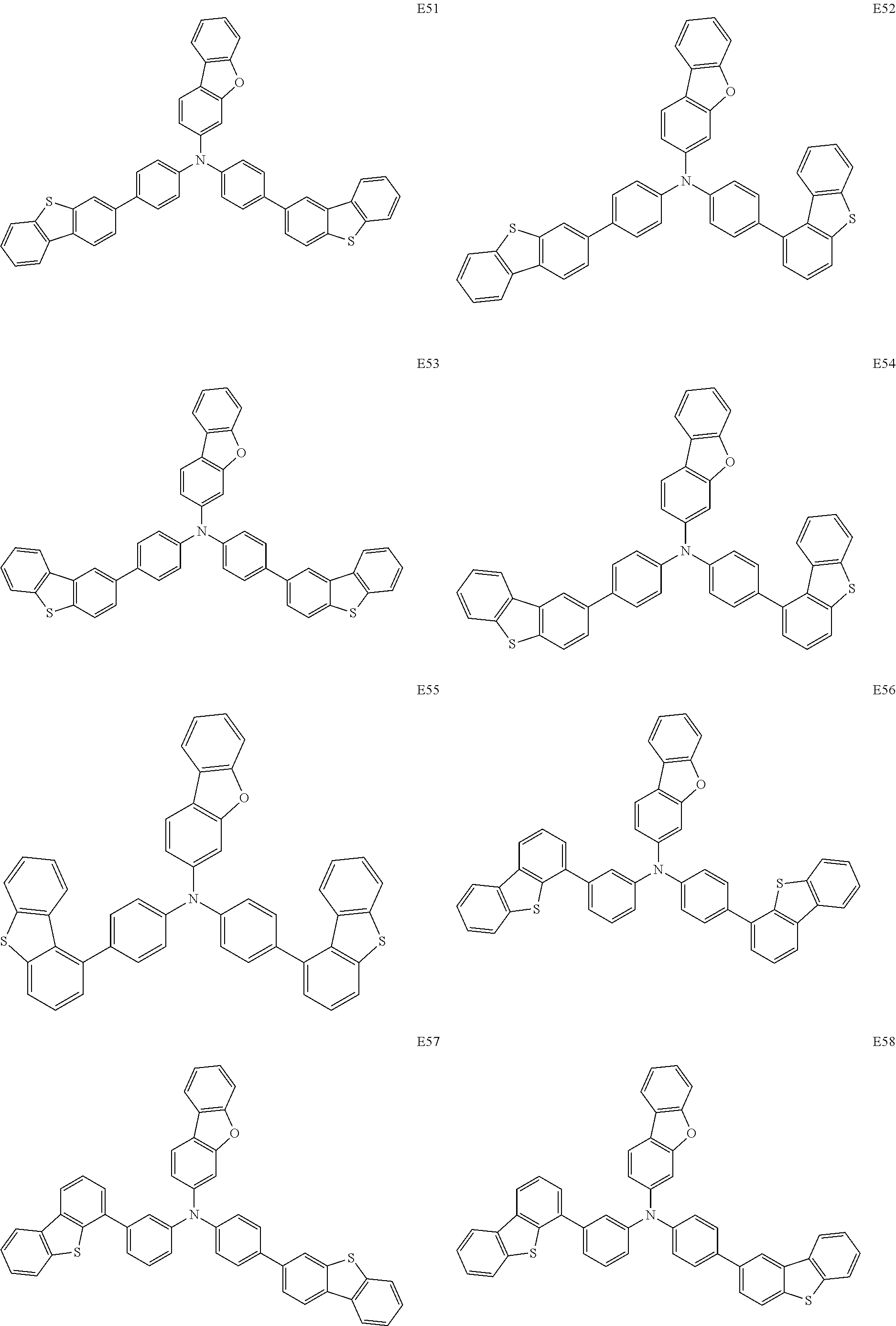

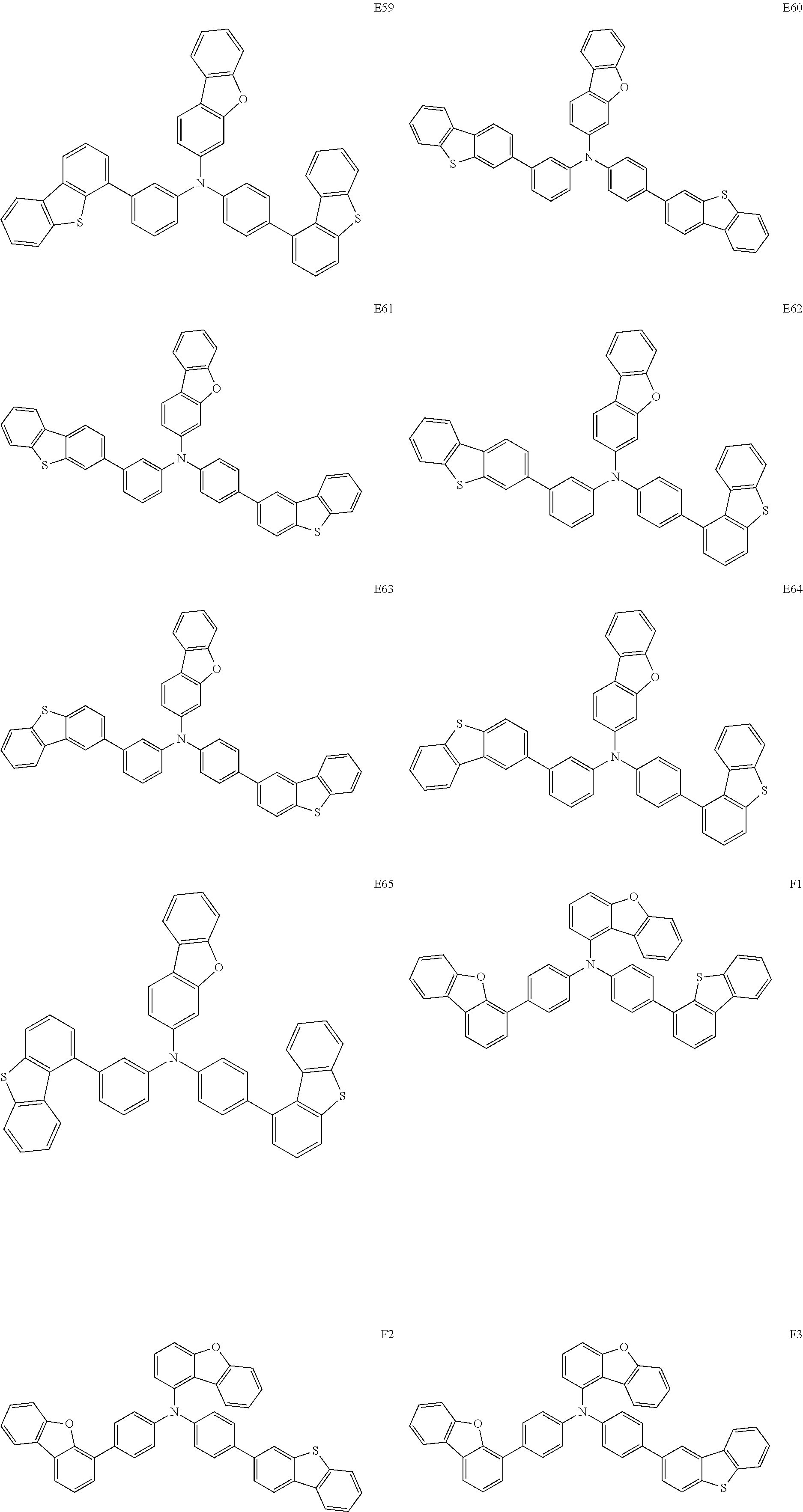

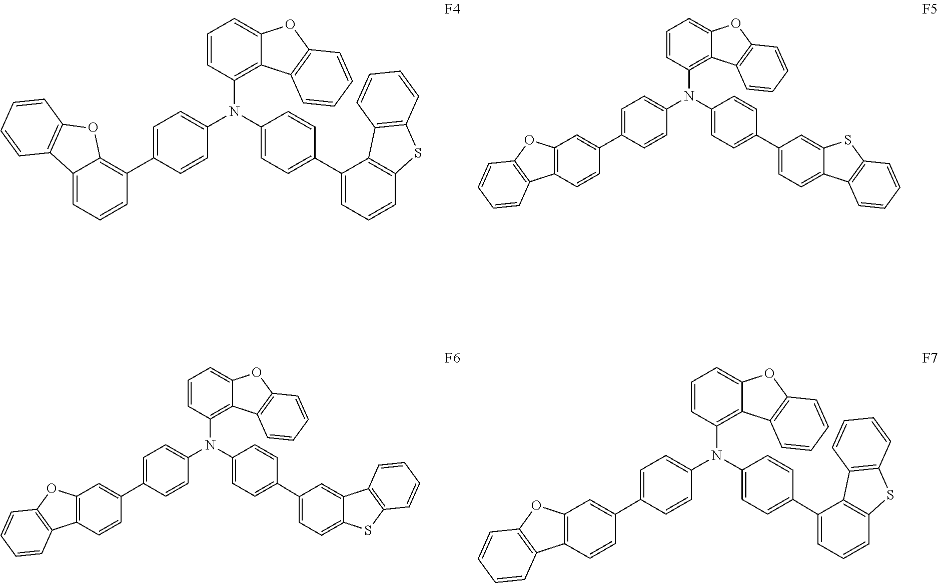 [SMILES: c1ccc2c(c1)oc1c(-c3ccc(N(c4ccc(-c5cccc6sc7ccccc7c56)cc4)c4cccc5oc6ccccc6c45)cc3)cccc12 ; c1ccc2c(c1)oc1cc(-c3ccc(N(c4ccc(-c5ccc6c(c5)sc5ccccc56)cc4)c4cccc5oc6ccccc6c45)cc3)ccc12 ; c1ccc2c(c1)oc1cc(-c3ccc(N(c4ccc(-c5ccc6sc7ccccc7c6c5)cc4)c4cccc5oc6ccccc6c45)cc3)ccc12 ; c1ccc2c(c1)oc1cc(-c3ccc(N(c4ccc(-c5cccc6sc7ccccc7c56)cc4)c4cccc5oc6ccccc6c45)cc3)ccc12]

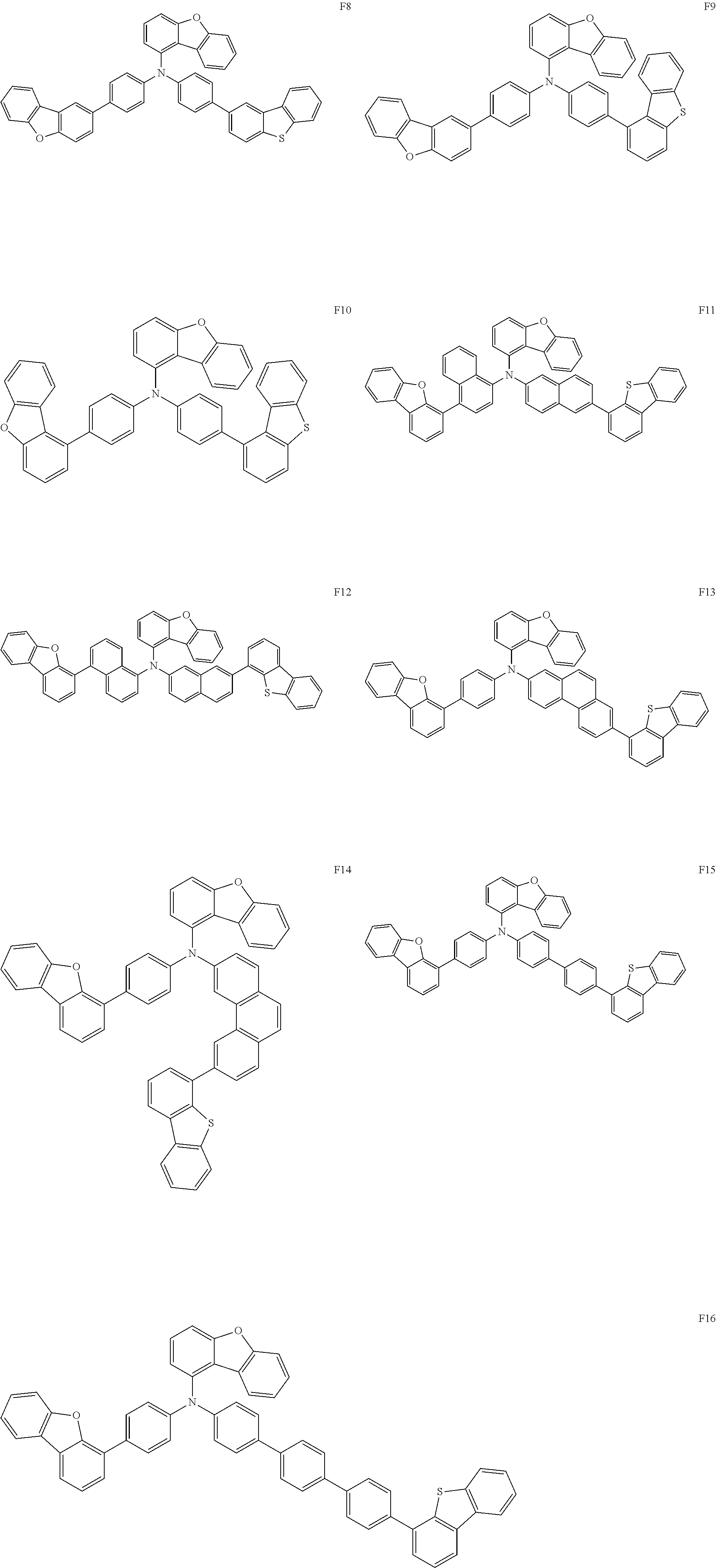

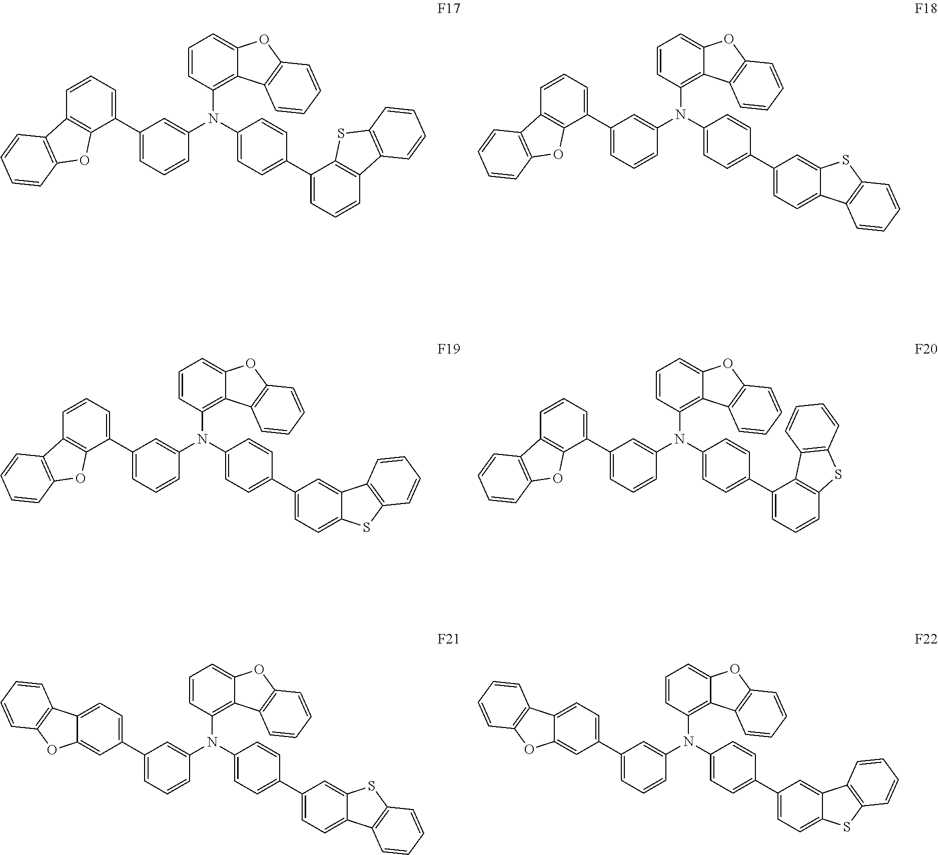

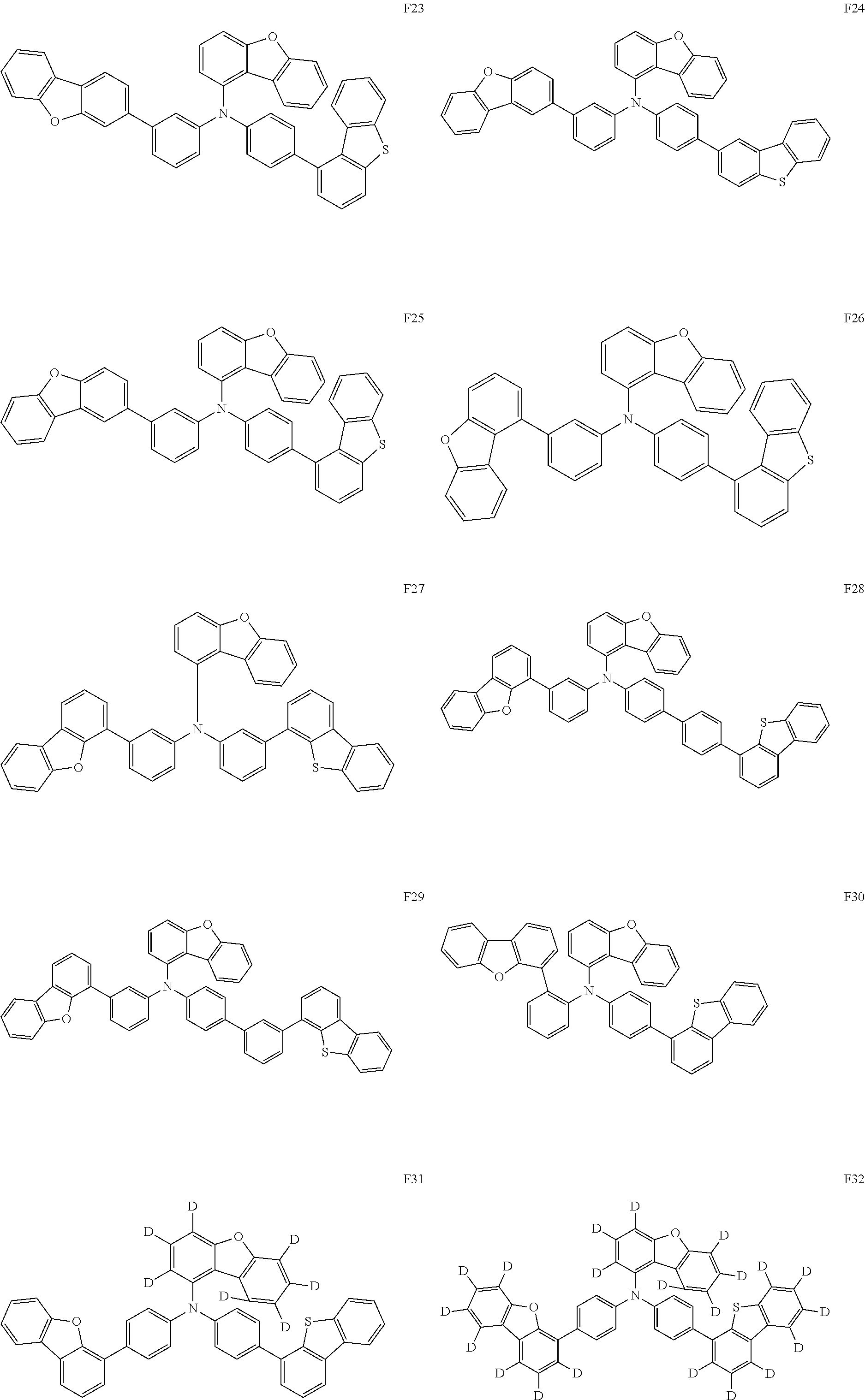

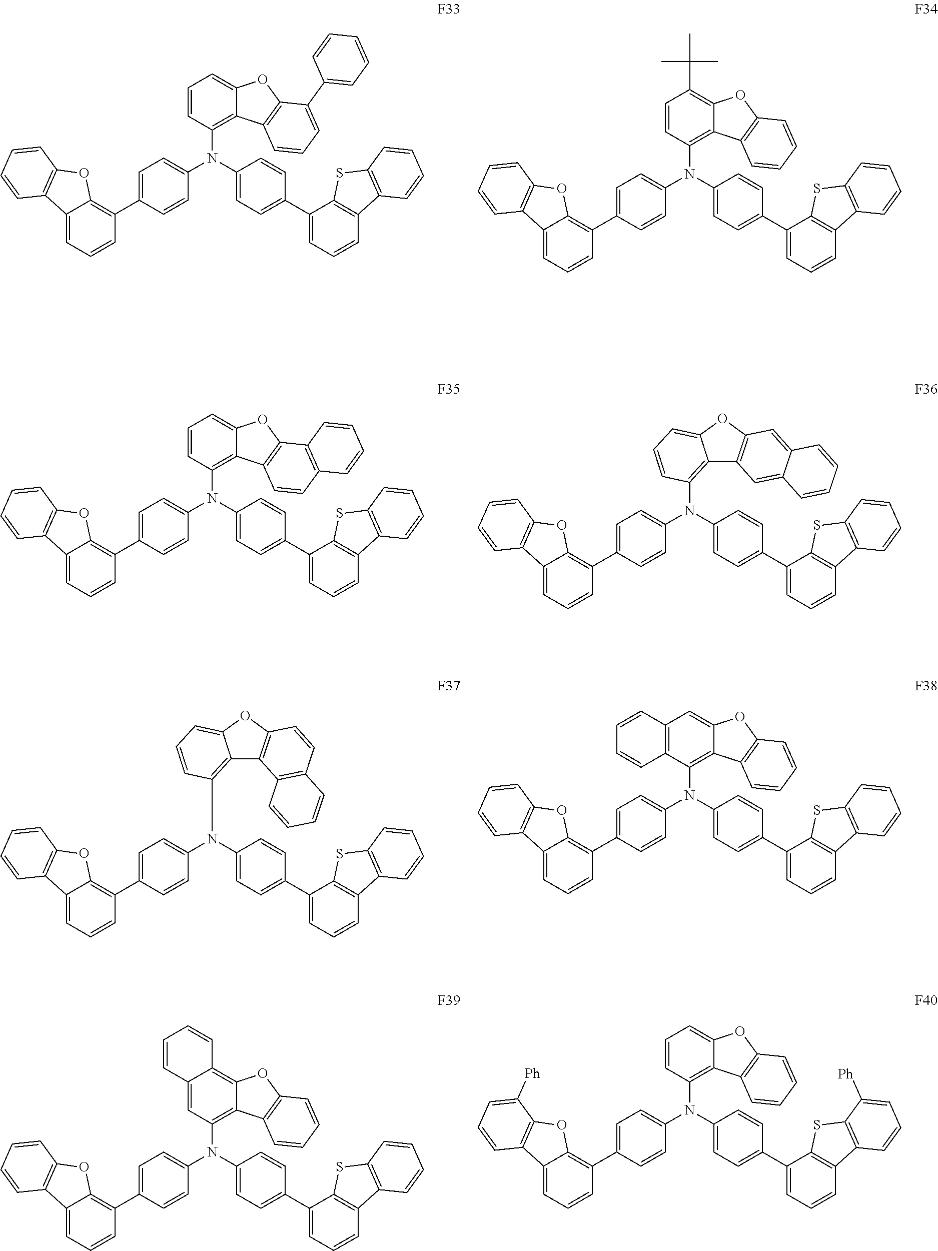

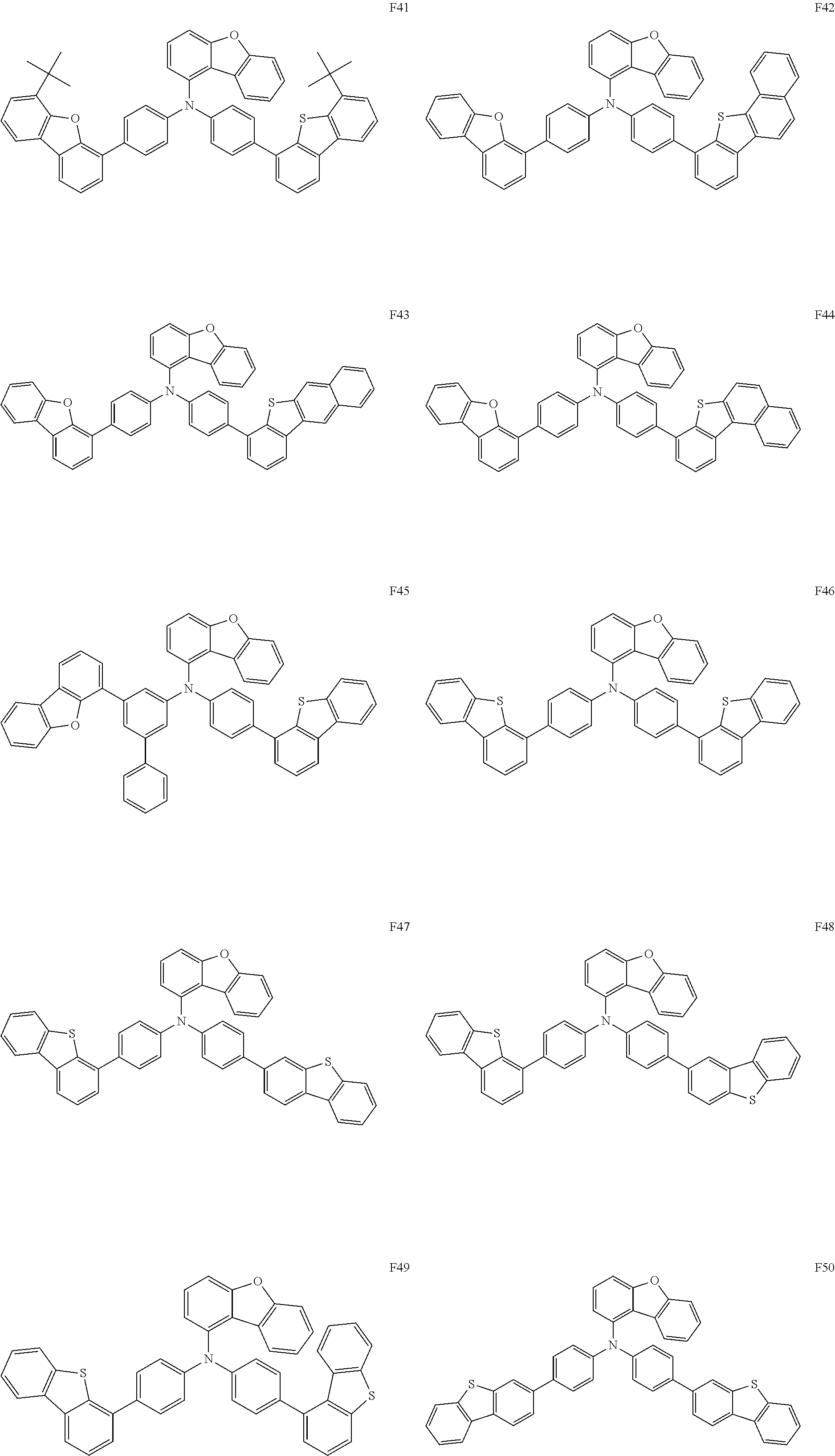

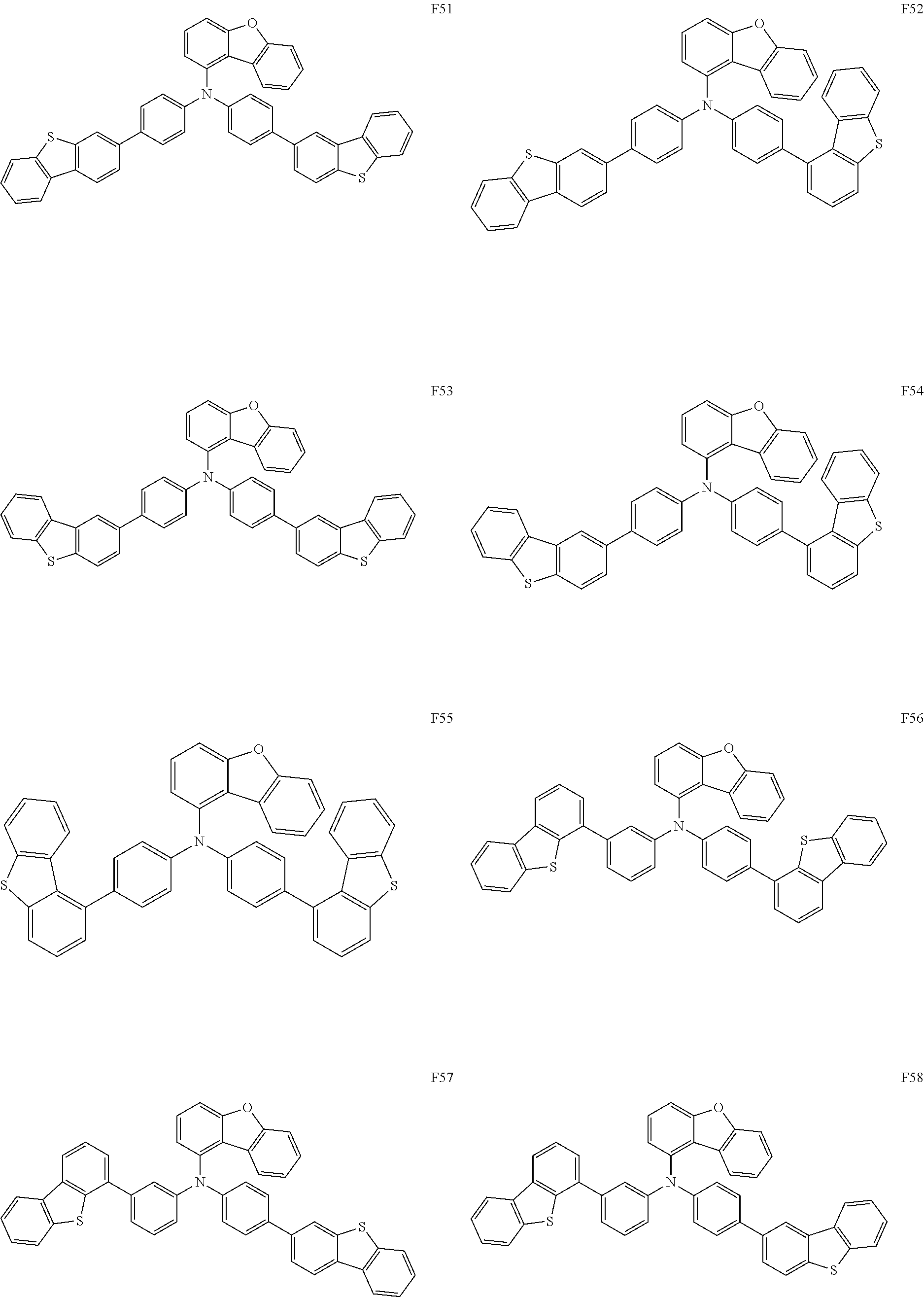

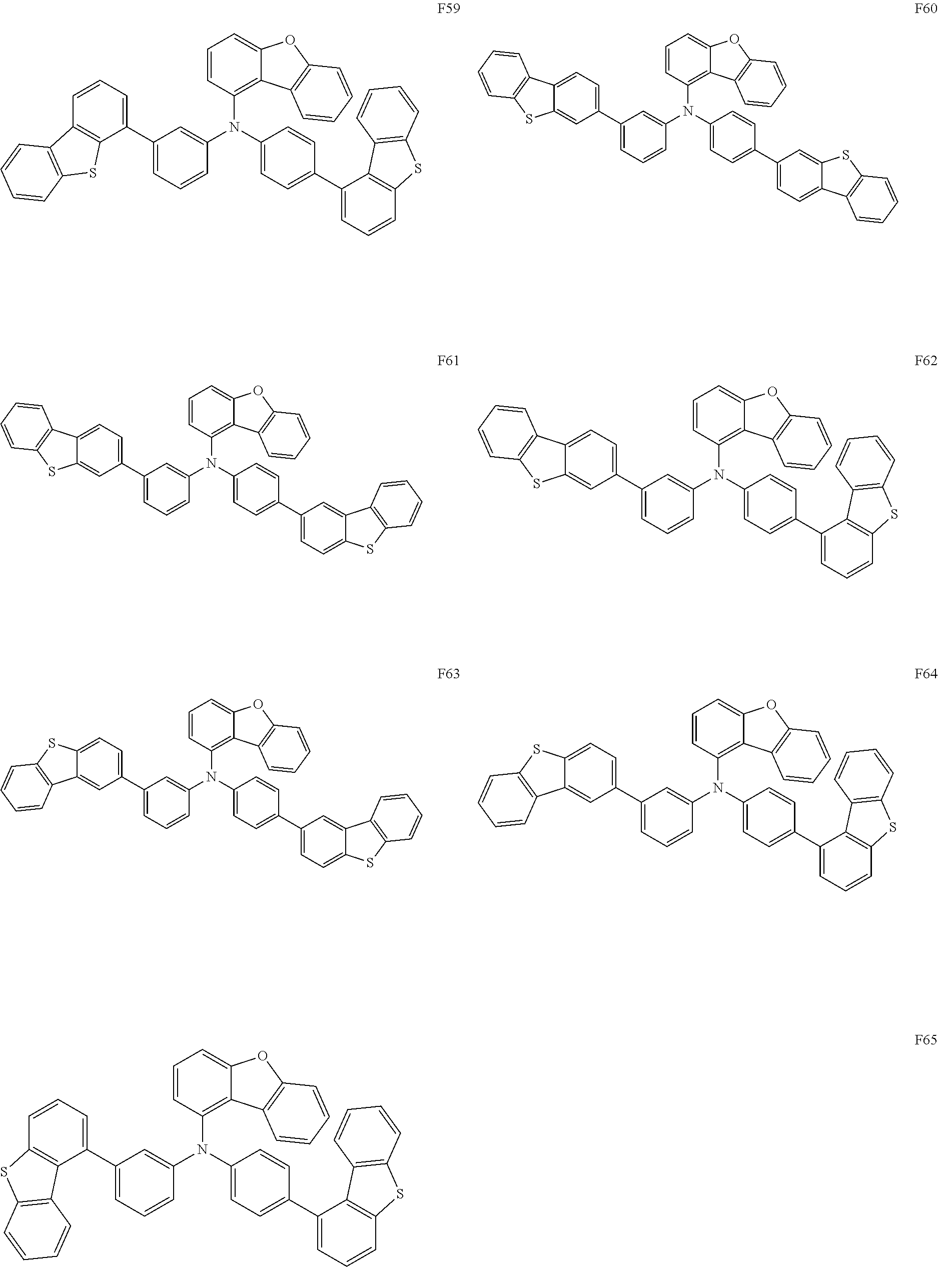

Referring to FIG. 3 to FIG. 6 again, the luminescence device ED according to an embodiment will be explained.

As described above, the hole transport region HTR may include the amine compound according to an embodiment. For example, the hole transport region HTR may include the amine compound represented by Formula 1.

If the hole transport region HTR has a multilayer structure having multiple layers, any one layer among the multiple layers may include the amine compound represented by Formula 1. For example, the hole transport region HTR may include a hole injection layer HIL disposed on the first electrode EL1 and a hole transport layer HTL disposed on the hole injection layer HIL, and the hole transport layer HTL may include the amine compound represented by Formula 1. However, embodiments are not limited thereto. For example, the hole injection layer HIL may include the amine compound represented by Formula 1.

The hole transport region HTR may include one type, or two or more types of the amine compound represented by Formula 1. For example, the hole transport region HTR may include at least one selected from the compounds represented in Compound Group 1 and Compound Group 2.

The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

The hole injection region HIL may include, for example, a phthalocyanine compound such as copper phthalocyanine; N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(1-naphthyl)-N-phenylamino]-triphenylamine (1-TNATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f:2′,3′-h] quinoxaline-2,3,6,7,10,11-hexacarbonitrile (HAT-CN), etc.

The hole transport layer HTL may further include, for example, carbazole derivatives such as N-phenyl carbazole and polyvinyl carbazole, fluorene-based derivatives, N,N′-bis(3-methylphenyl)-N,N′-diphenyl-[1,1′-biphenyl]-4,4′-diamine (TPD), triphenylamine-based derivatives such as 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl)benzeneamine (TAPC), 4,4′-bis[N,N′-(3-tolyl)amino]-3,3′-dimethylbiphenyl (HMTPD), 1,3-bis(N-carbazolyl)benzene (mCP), etc.

The electron blocking layer EBL may include, for example, carbazole derivatives such as N-phenyl carbazole and polyvinyl carbazole, fluorene-based derivatives, N,N′-bis(3-methylphenyl)-N,N′-diphenyl-[1,1′-biphenyl]-4,4′-diamine (TPD), triphenylamine-based derivatives such as 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 9-(4-tert-butylphenyl)-3,6-bis(triphenylsilyl)-9H-carbazole (CzSi), 9-phenyl-9H-3,9′-bicarbazole (CCP), 1,3-bis(N-carbazolyl)benzene (mCP), 1,3-bis(1,8-dimethyl-9H-carbazol-9-yl)benzene (mDCP), etc.

The hole transport region HTR may further include a compound represented by Formula H-1 below.

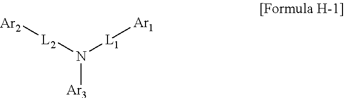

In Formula H-1, L₁ and L₂ may each independently be a direct linkage, a substituted or unsubstituted arylene group of 6 to 30 ring-forming carbon atoms, or a substituted or unsubstituted heteroarylene group of 2 to 30 ring-forming carbon atoms. In Formula H-1, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. In Formula H-1, Ar₃ may be a substituted or unsubstituted aryl group of 6 to 30 ring-forming carbon atoms or a substituted or unsubstituted heteroaryl group of 2 to 30 ring-forming carbon atoms.

The compound represented by Formula H-1 may be a monoamine compound. Otherwise, the compound represented by Formula H-1 may be a diamine compound in which at least one among Ar₁ to Ar₃ includes an amine group as a substituent. Further, the compound represented by Formula H-1 may be a carbazole-based compound in which a substituted or unsubstituted carbazole group is included in at least one among Ar₁ and Ar₂, or a fluorene-based compound in which a substituted or unsubstituted fluorene group is included in at least one among Ar₁ and Ar₂.

The compound represented by Formula H-1 may be represented by any one among the compounds represented in Compound Group H below. However, the compounds illustrated in Compound Group H are only embodiments, and the compound represented by Formula H-1 is not limited to the compounds represented in Compound Group H below.

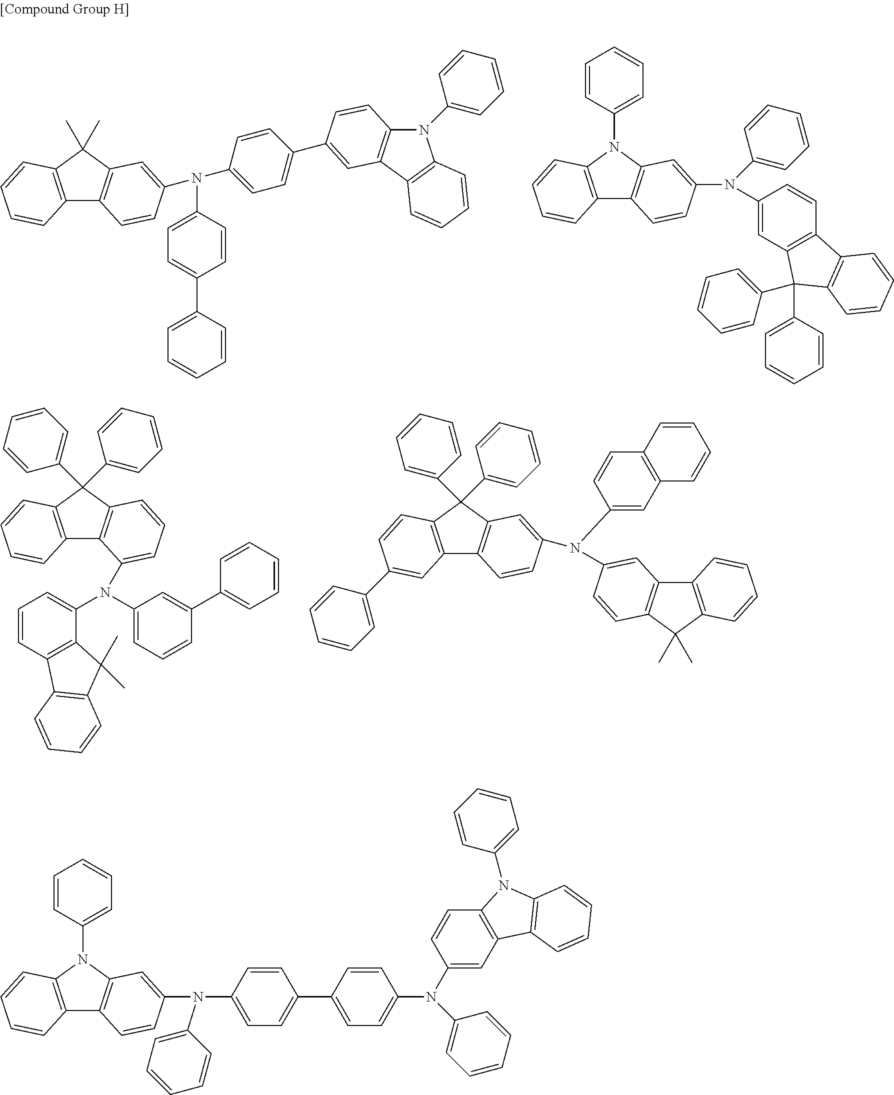

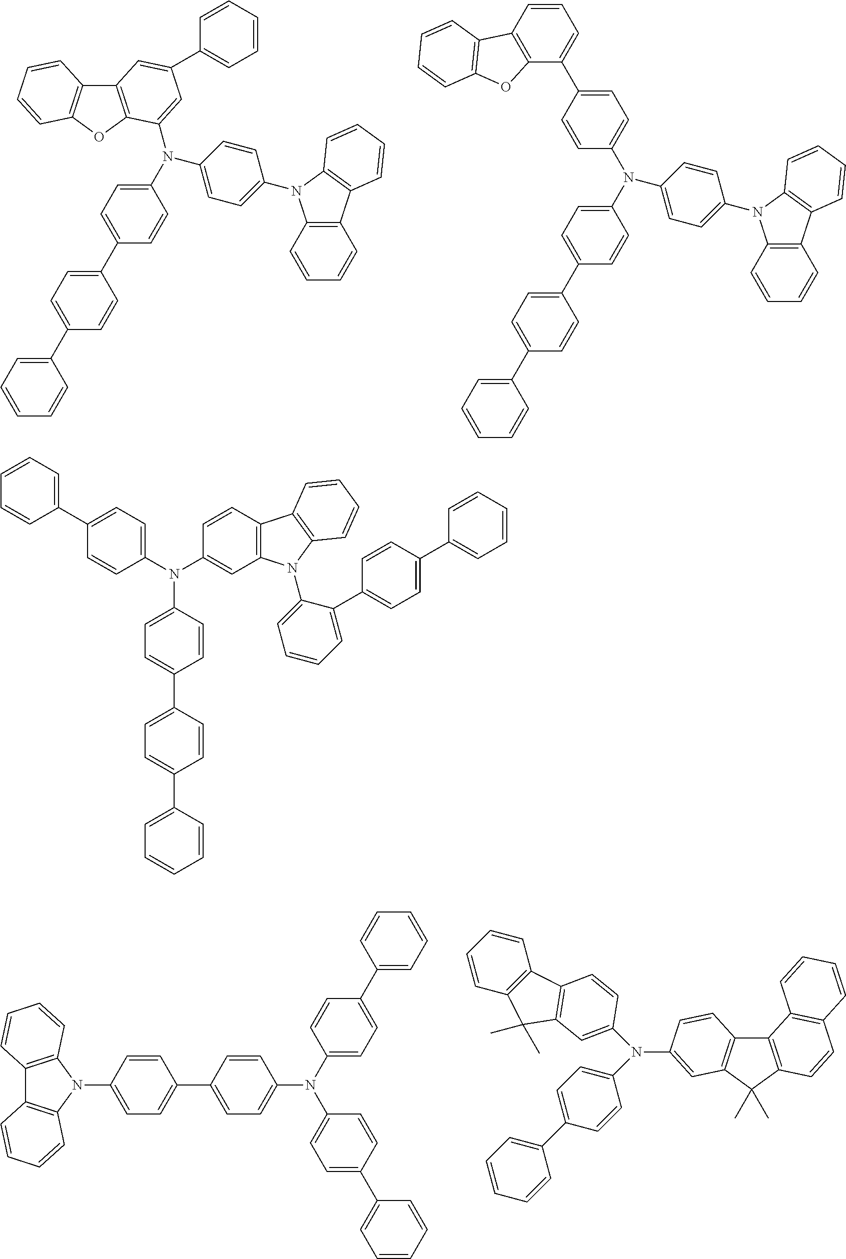

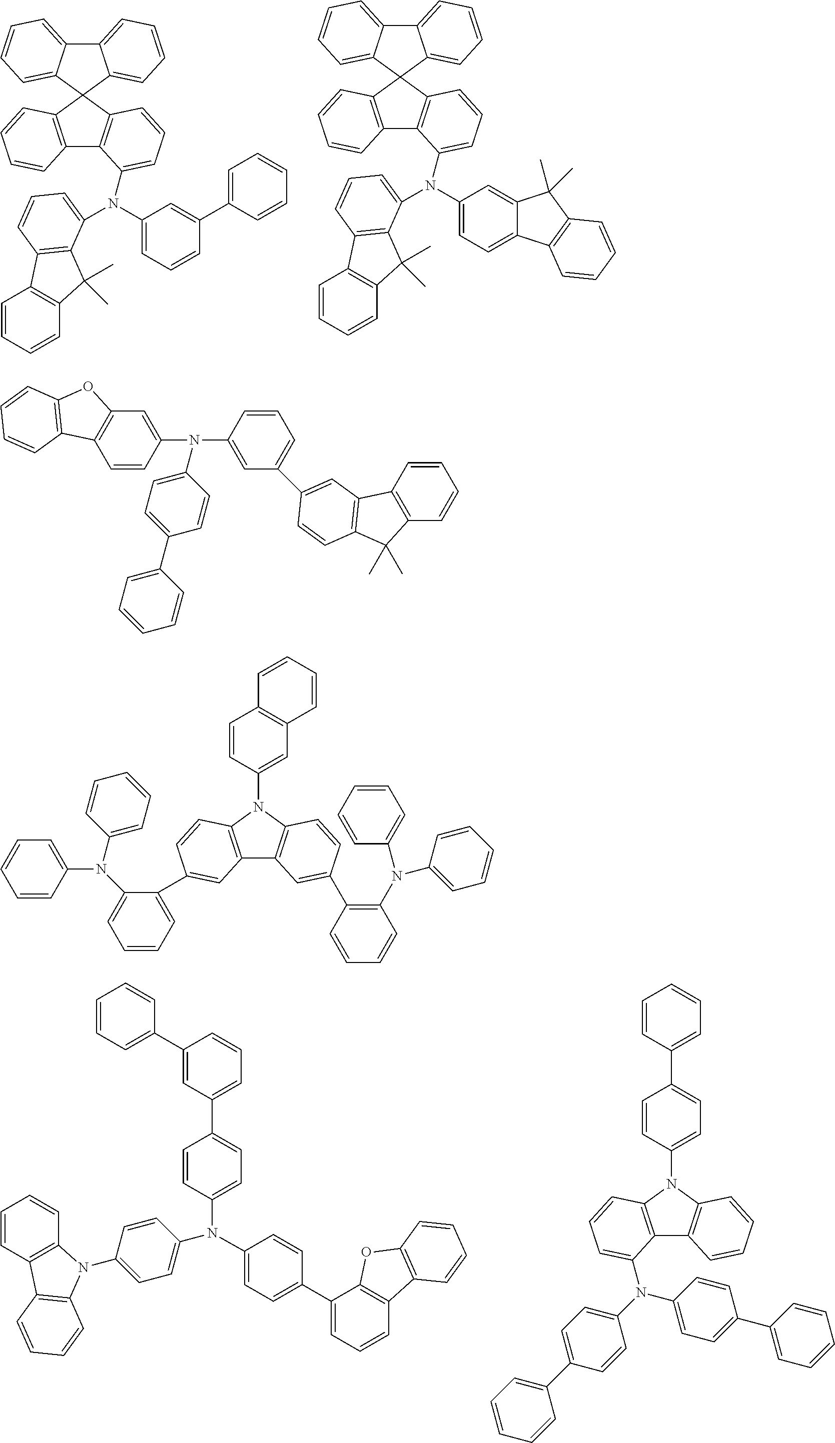

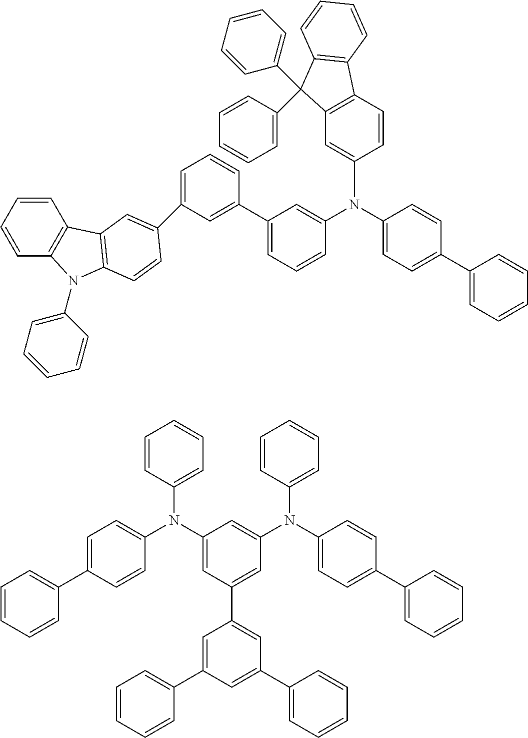 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1]

In an embodiment, a thickness of the hole transport region HTR may be in a range of about 100 Å to about 10,000 Å. For example, the thickness of the hole transport region HTR may be in a range of about 100 Å to about 5,000 Å. A thickness of the hole injection region HIL may be, for example, in a range of about 30 Å to about 1,000 Å, and a thickness of the hole transport layer HTL may be in a range of about 30 Å to about 1,000 Å. For example, a thickness of the electron blocking layer EBL may be in a range of about 10 Å to about 1,000 Å. If the thicknesses of the hole transport region HTR, the hole injection layer HIL, the hole transport layer HTL and the electron blocking layer EBL satisfy the above-described ranges, satisfactory hole transport properties may be obtained without substantial increase of a driving voltage.

The hole transport region HTR may further include a charge generating material in addition to the above-described materials to increase conductivity. The charge generating material may be dispersed uniformly or non-uniformly in the hole transport region HTR. The charge generating material may be, for example, a p-dopant. The p-dopant may be any one among quinone derivatives, metal oxides, and cyano group-containing compounds, without limitation. For example, non-limiting examples of the p-dopant may include quinone derivatives such as tetracyanoquinodimethane (TCNQ) and 2,3,5,6-tetrafluoro-7,7′,8,8-tetracyanoquinodimethane (F4-TCNQ), metal oxides such as tungsten oxide, and molybdenum oxide, etc., without limitation.

As described above, the hole transport region HTR may further include at least one of a hole buffer layer (now shown) and an electron blocking layer EBL in addition to the hole injection layer HIL and the hole transport layer HTL. The hole buffer layer (not shown) may compensate an optical resonance distance according to the wavelength of light emitted from an emission layer EML and may increase light emission efficiency. Materials which may be included in a hole transport region HTR may be used as materials included in a hole buffer layer (not shown). The electron blocking layer EBL is a layer that may prevent electron injection from the electron transport region ETR to the hole transport region HTR.

The emission layer EML is provided on the hole transport region HTR. For example, the emission layer EML may have a thickness in a range of about 100 Å to about 1,000 Å. For example, the thickness of the emission layer EML may be in a range of about 100 Å to about 300 Å. The emission layer EML may have a single layer formed using a single material, a single layer formed using different materials, or a multilayer structure having multiple layers formed using different materials.

In the luminescence device ED of an embodiment, the emission layer EML may include anthracene derivatives, pyrene derivatives, fluoranthene derivatives, chrysene derivatives, dihydrobenzanthracene derivatives, or triphenylene derivatives. For example, the emission layer EML may include anthracene derivatives or pyrene derivatives.

In the luminescence devices ED of embodiments, shown in FIG. 3 to FIG. 6, the emission layer EML may include a host and a dopant, and the emission layer EML may include a compound represented by Formula E-1 below. The compound represented by Formula E-1 below may be used as a fluorescence host material.

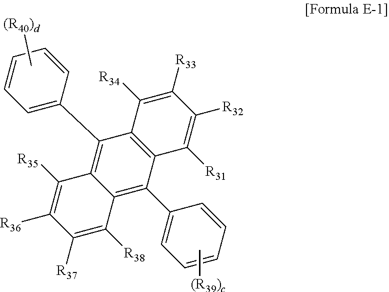

In Formula E-1, R₃₁ to R₄₀ may each independently be a hydrogen atom, a deuterium atom, a halogen atom, a substituted or unsubstituted silyl group, a substituted or unsubstituted alkyl group of 1 to 10 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula E-1, R₃₁ to R₄₀ may be combined with an adjacent group to form a saturated hydrocarbon ring or an unsaturated hydrocarbon ring.

In Formula E-1, c and d may each independently be an integer from 0 to 5.

The compound represented by Formula E-1 may be selected from any one among Compound E1 to Compound E19 below.

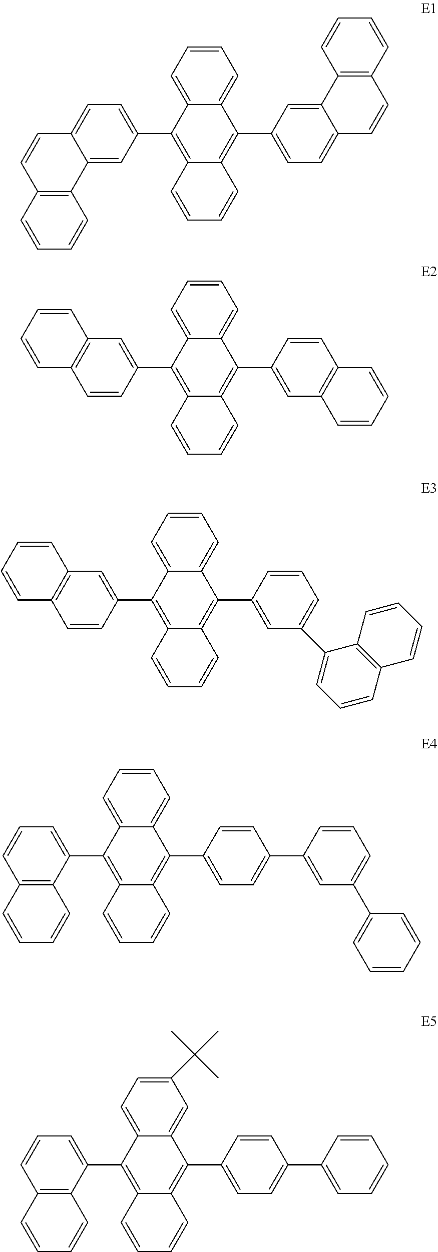

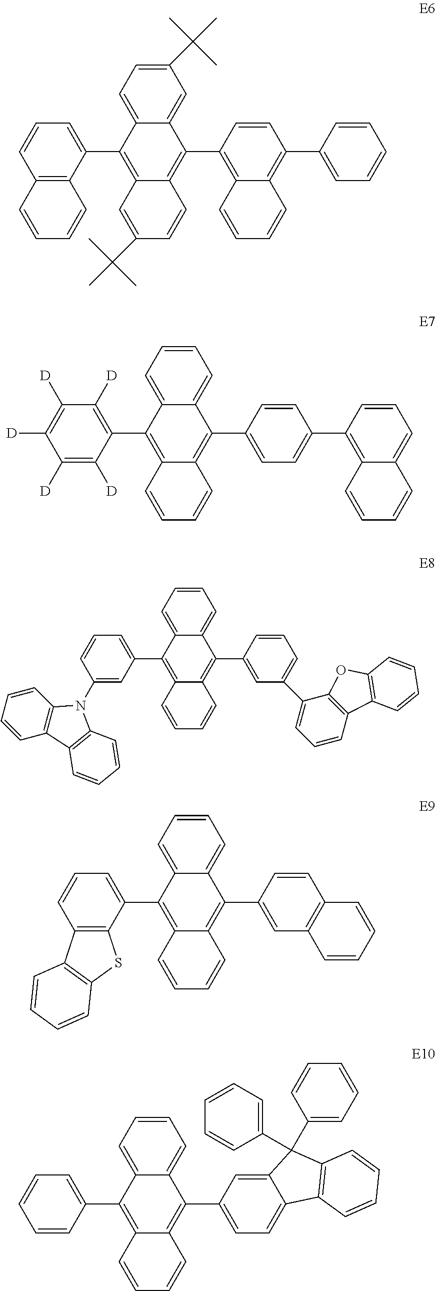

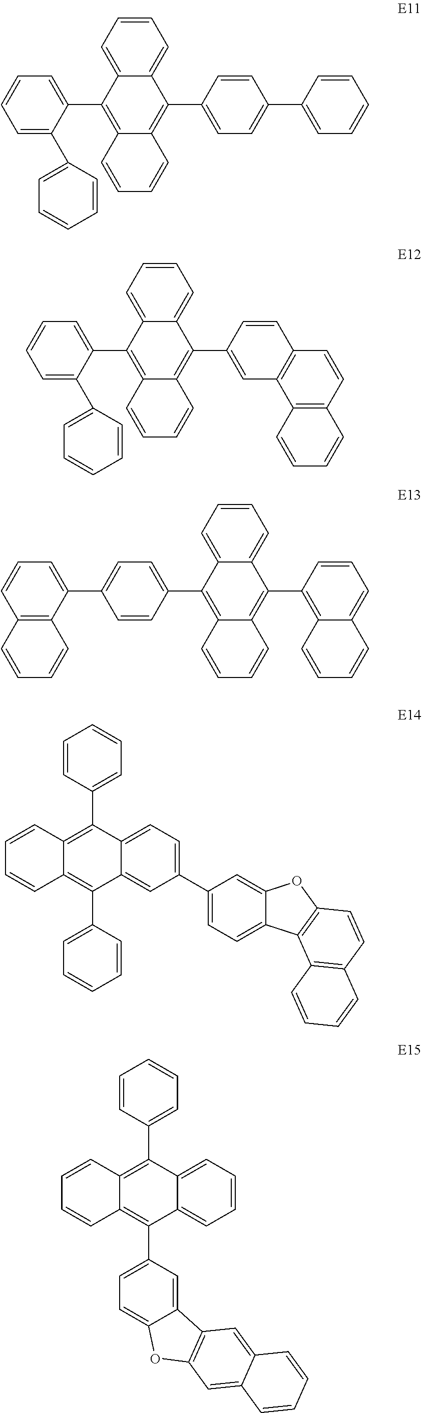

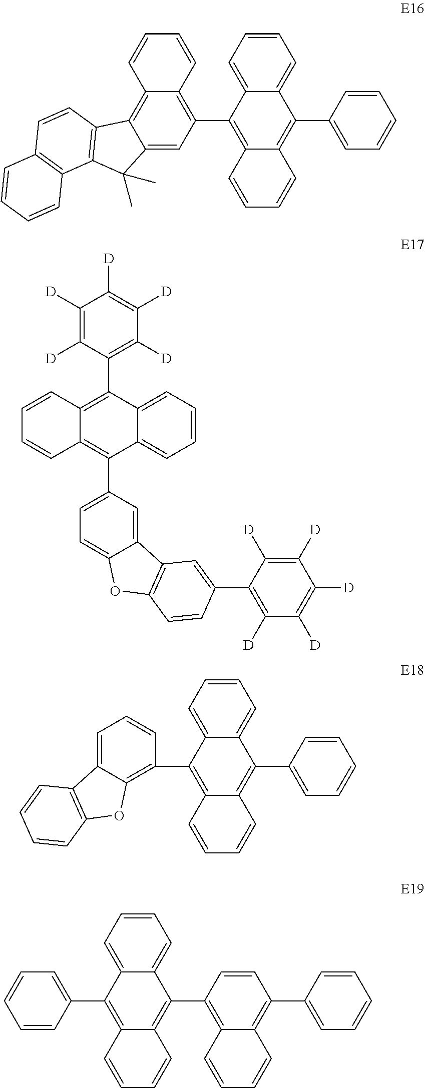

In an embodiment, the emission layer EML may include a compound represented by Formula E-2a or Formula E-2b below. The compound represented by Formula E-2a or Formula E-2b may be used as a phosphorescence host material.

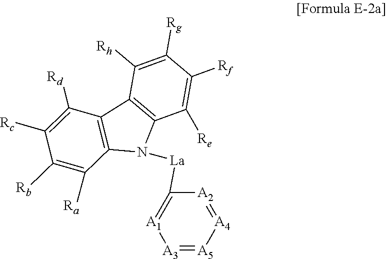

In Formula E-2a, Lₐ may be a direct linkage or a substituted or unsubstituted arylene group of 6 to 30 ring-forming carbon atoms. In Formula E-2a, A₁ to A₅ may each independently be N or C(Ri). Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group with each other to form a ring. Rₐ to Rᵢ may be combined with an adjacent group to form a hydrocarbon ring or a heterocycle including N, O, S, etc. as a ring-forming atom.

In Formula E-2a, two or three of A₁ to A₅ may be N, and the remainder of A₁ to A₅ may be C(Rᵢ).

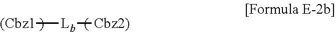

In Formula E-2b, Cbz1 and Cbz2 may each independently be an unsubstituted carbazole group, or a carbazole group substituted with an aryl group of 6 to 30 ring-forming carbon atoms. L_{b} may be a direct linkage, or a substituted or unsubstituted arylene group of 6 to 30 ring-forming carbon atoms.

The compound represented by Formula E-2a or Formula E-2b may be represented by any one among the compounds in Compound Group E-2 below. However, the compounds shown in Compound Group E-2 below are only illustrations, and the compound represented by Formula E-2a or Formula E-2b is not limited to the compounds represented in Compound Group E-2 below.

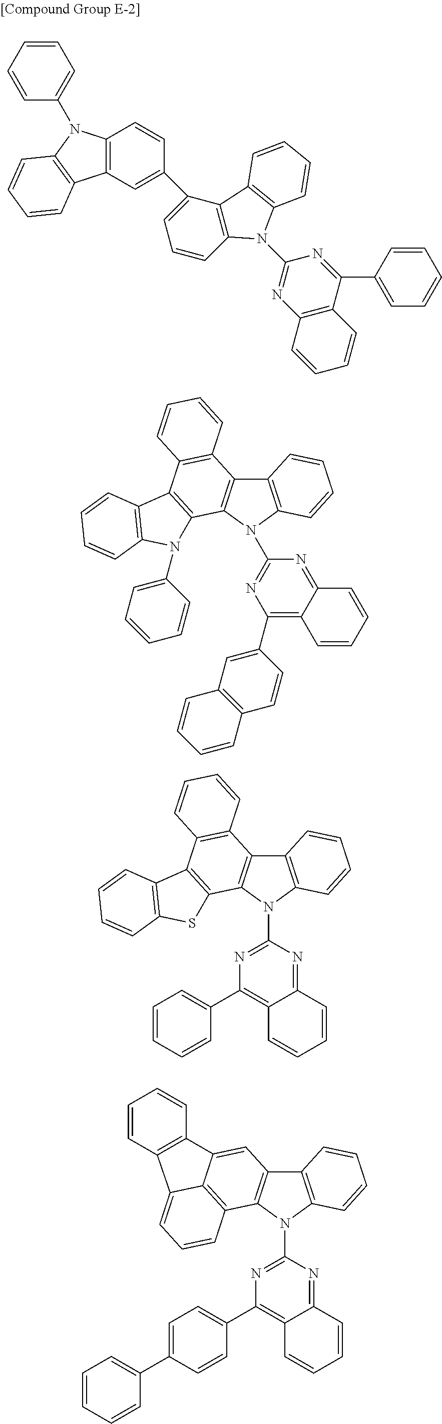

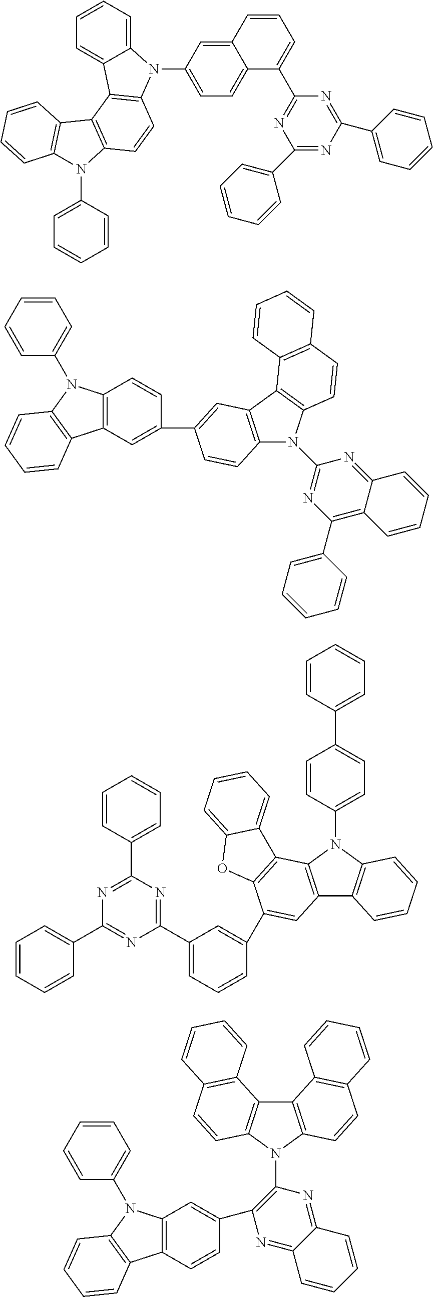

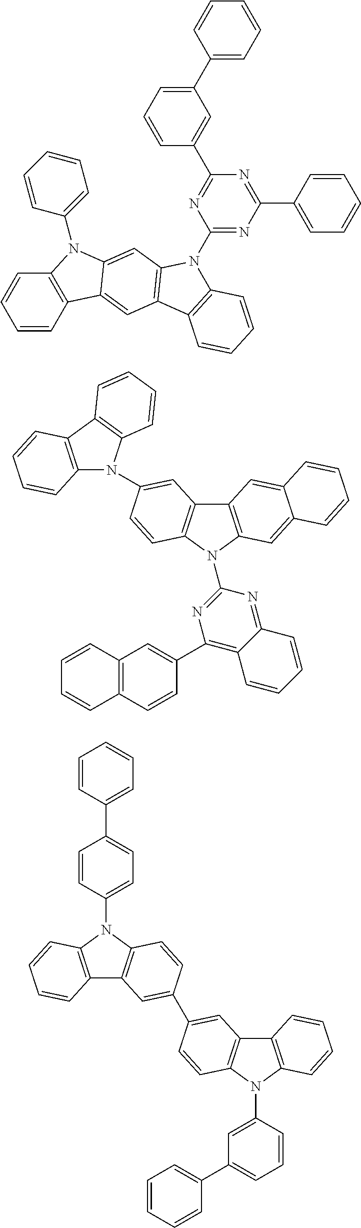

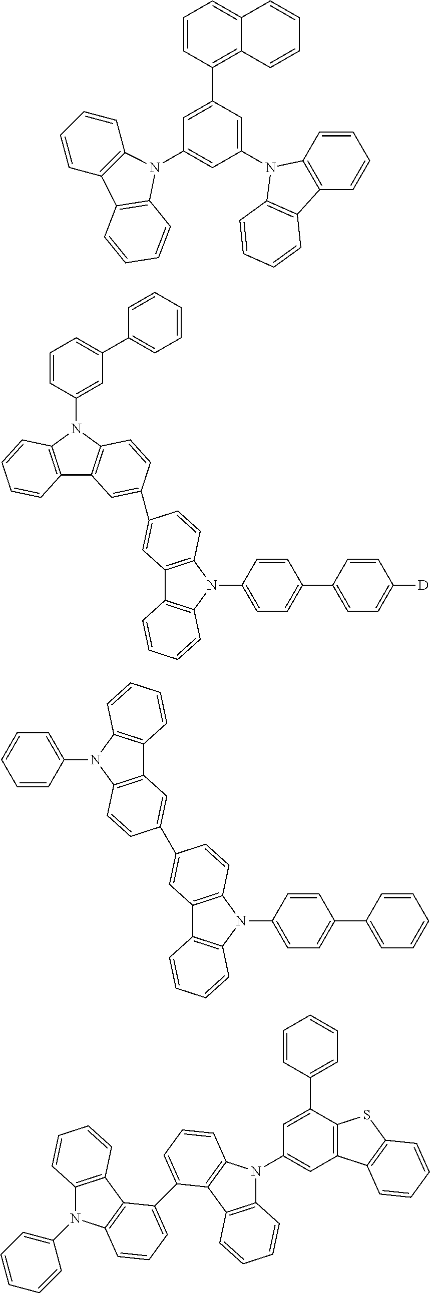

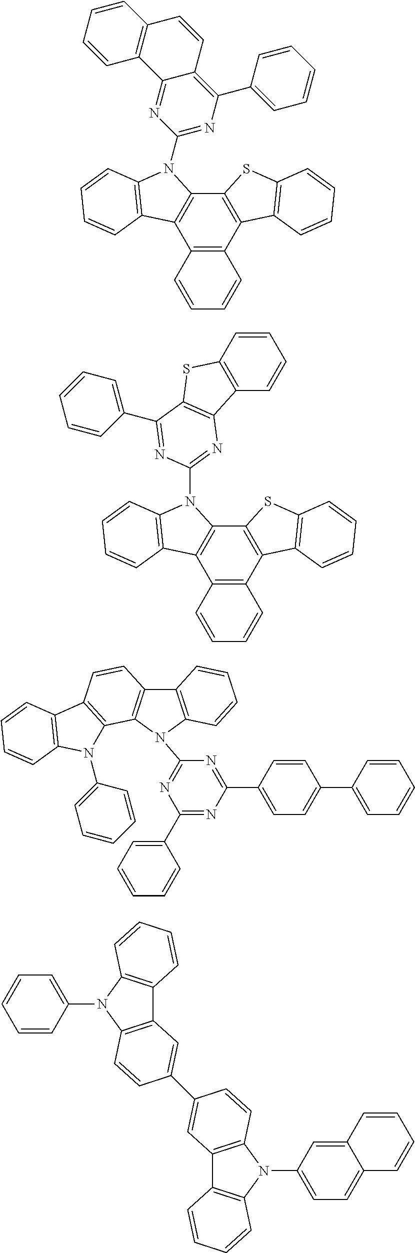

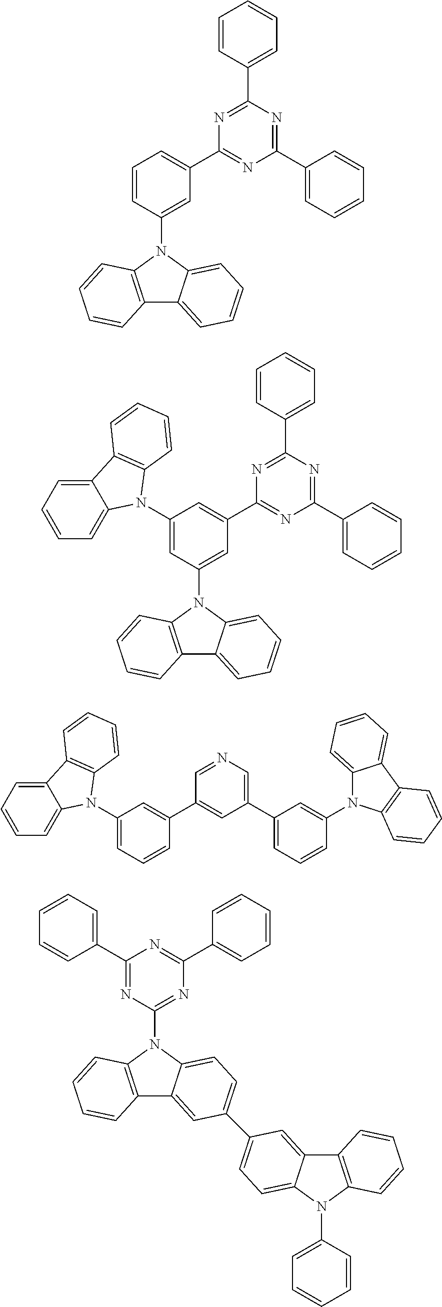 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a common material in the art as a host material. For example, the emission layer EML may include as a host material, at least one of bis[2-(diphenylphosphino)phenyl] ether oxide (DPEPO), 4,4′-bis(N-carbazol-9-yl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), and 1,3,5-tris(1-phenyl-1H-benzo[d]imidazole-2-yl)benzene (TPBi). However, embodiments are not limited thereto. For example, tris(8-hydroxyquinolino)aluminum (Alq₃), poly(N-vinylcarbazole) (PVK), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as the host material.

The emission layer EML may include a compound represented by Formula M-a or Formula M-b below. The compound represented by Formula M-a or Formula M-b may be used as a phosphorescence dopant material.

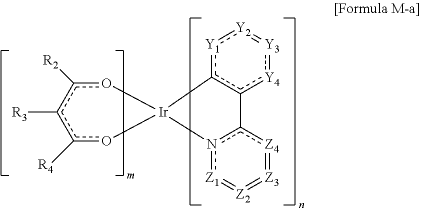

In Formula M-a, Y₁ to Y₄ and Z₁ to Z₄ may each independently be C(R₁) or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula M-a, m may be 0 or 1, and n may be 2 or 3. In Formula M-a, if m is 0, n may be 3, and if m is 1, n may be 2.

The compound represented by Formula M-a may be used as a red phosphorescence dopant or a green phosphorescence dopant.

The compound represented by Formula M-a may be represented by any one among Compounds M-a1 to M-a19 below. However, Compounds M-a1 to M-a19 below are only illustrations, and the compound represented by Formula M-a is not limited to the compounds represented by Compounds M-a1 to M-a19 below.

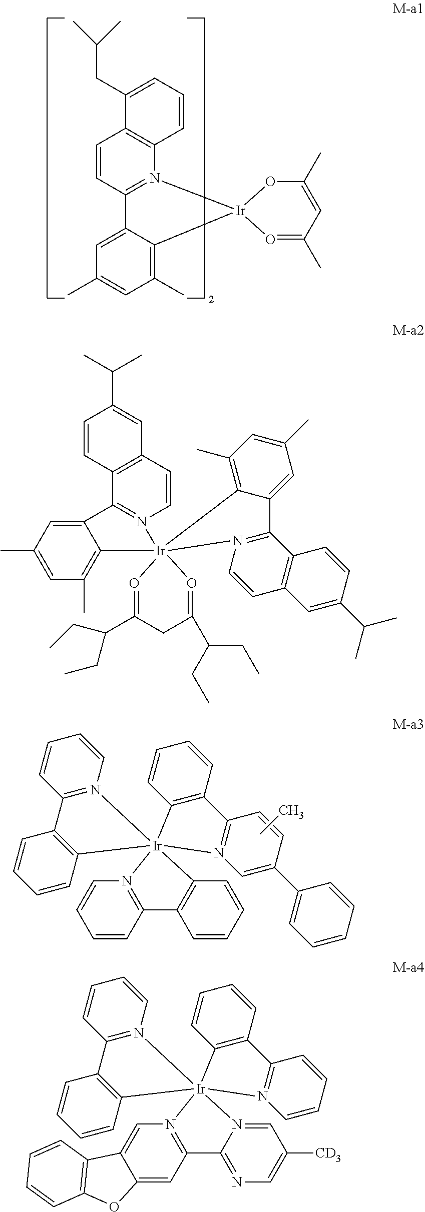

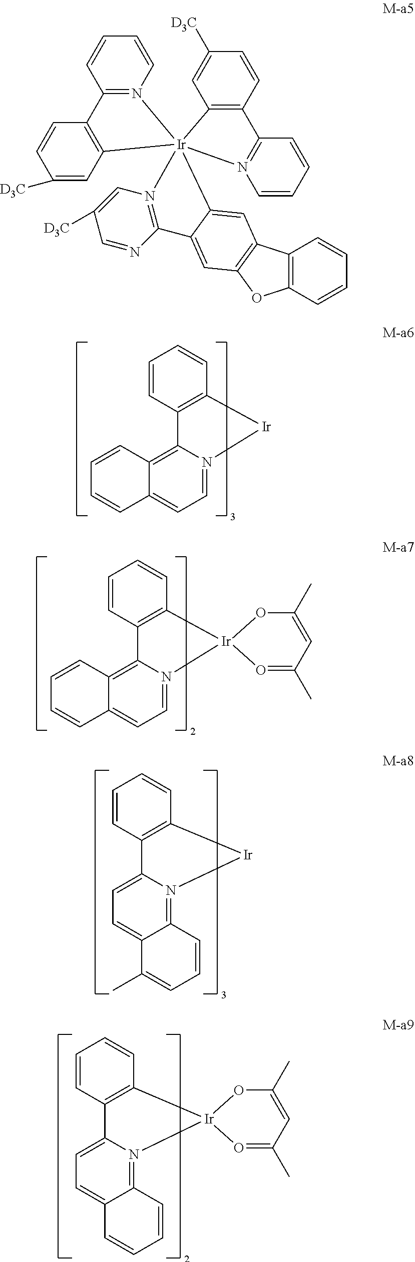

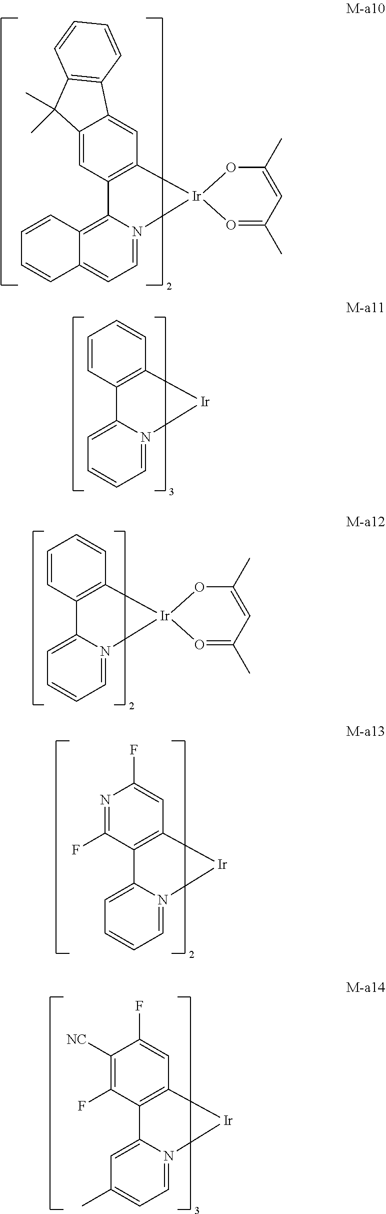

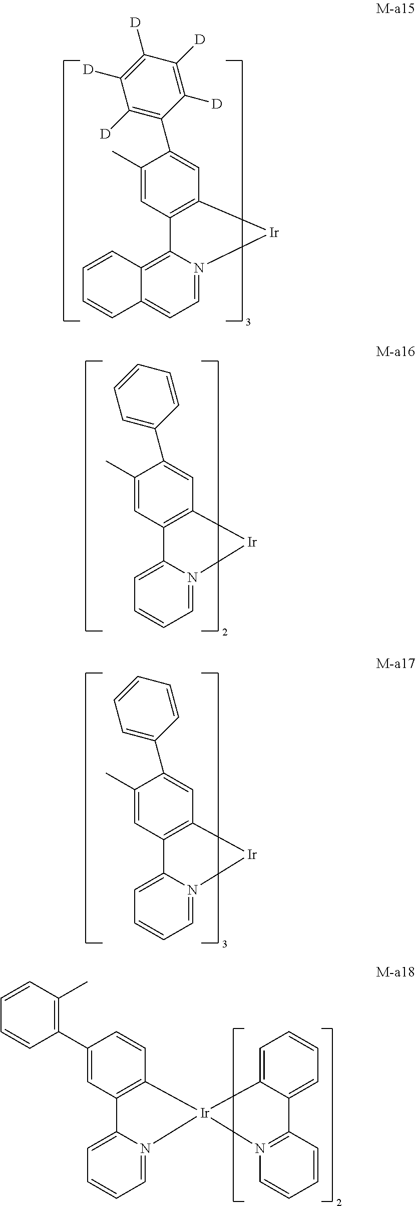

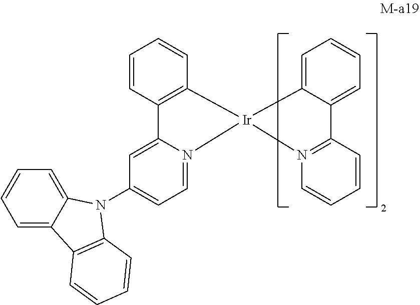

Compound M-a1 and Compound M-a2 may be used as red dopant materials, and Compound M-a3 to Compound M-a5 may be used as green dopant materials.

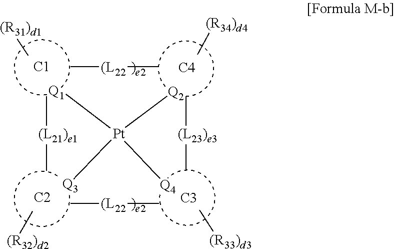

In Formula M-b, Q₁ to Q₄ may each independently be C or N, C1 to C4 may each independently be a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ may each independently be a direct linkage,

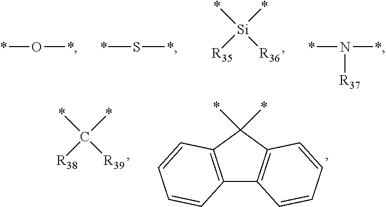

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring, and d1 to d4 may each independently be an integer from 0 to 4.

The compound represented by Formula M-b may be used as a blue phosphorescence dopant or a green phosphorescence dopant.

The compound represented by Formula M-b may be represented by any one among the compounds below. However, the compounds below are only illustrations, and the compound represented by Formula M-b is not limited to the compounds represented below.

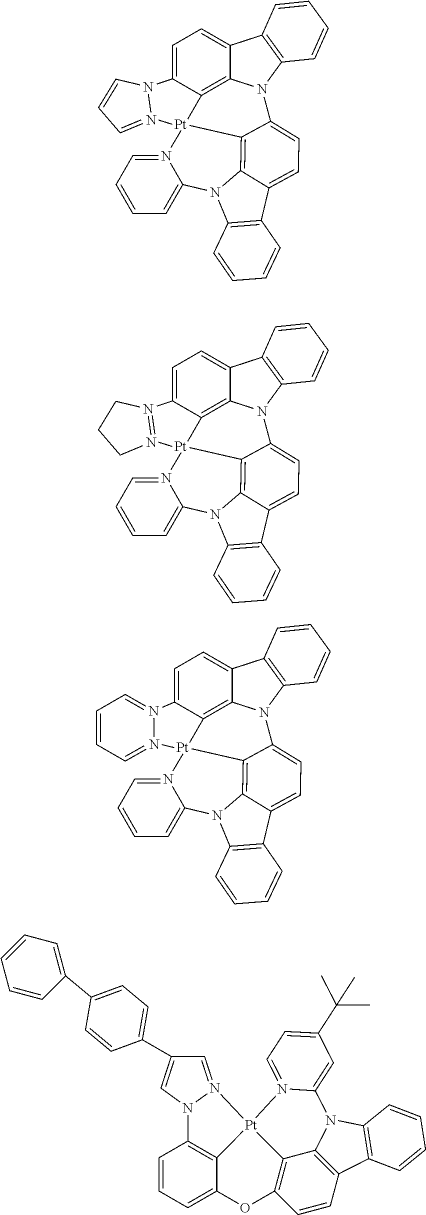

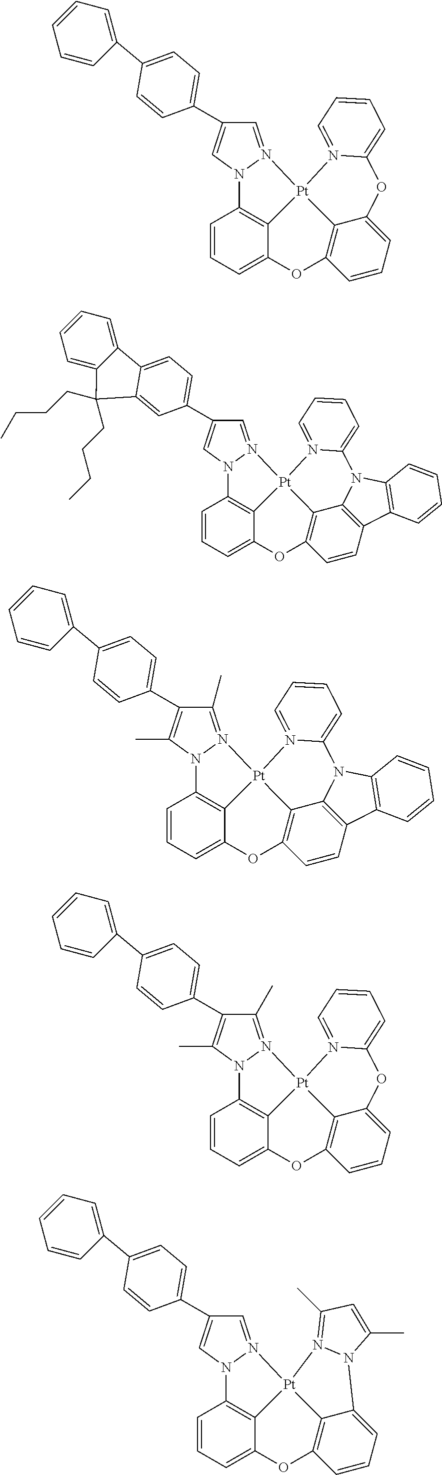

The emission layer EML may include a compound represented by any one among Formula F-a to Formula F-c below. The compounds represented by Formula F-a to Formula F-c below may be used as fluorescence dopant materials.

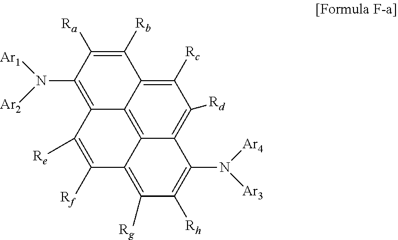

In Formula F-a, Rₐ to Rₕ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. An to Ara may each independently be a substituted or unsubstituted aryl group of 6 to 30 ring-forming carbon atoms, or a substituted or unsubstituted heteroaryl group of 2 to 30 ring-forming carbon atoms. For example, at least one among Ar₁ to Ar₄ may be a heteroaryl group including 0 or S as a ring-forming atom.

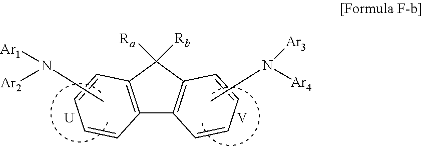

In Formula F-b, Rₐ and R_{b} may each independently be a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring.

In Formula F-b, U and V may each independently be 0 or 1. In Formula F-b, U means the number of rings bonded at the position of U, and V means the number of rings bonded at the position of V. For example, if U or V is 1, the ring marked with U or V may form a fused ring, and if U or V is 0, it means that no ring marked with U or V is present. For example, if U is 0 and V is 1, or if U is 1 and V is 0, a fused ring having a fluorene core of Formula F-b may be a ring compound having four rings. In an embodiment, if both U and V are 0, the fused ring of Formula F-b may be a ring compound having three rings. In an embodiment, if both U and V are 1, the fused ring having a fluorene core of Formula F-b may be a ring compound having five rings.

In Formula F-b, if U or V is 1, U and V may each independently be a substituted or unsubstituted hydrocarbon ring of 5 to 30 ring-forming carbon atoms, or a substituted or unsubstituted heterocycle of 2 to 30 ring-forming carbon atoms.

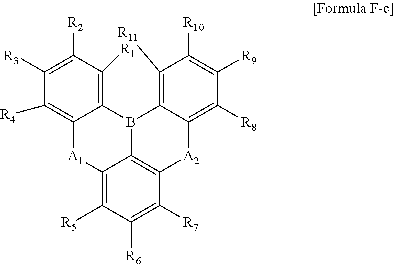

In Formula F-c, A₁ and A₂ may each independently be O, S, Se, or N(Rₘ), and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring.

In Formula F-c, A₁ and A₂ may each independently be combined with the substituents of an adjacent ring to form a fused ring. For example, if A₁ and A₂ are each independently N(Rₘ), A₁ may be combined with R₄ or R₅ to form a ring. In an embodiment, in Formula F-c, A₂ may be combined with R₇ or R₈ to form a ring.

In an embodiment, the emission layer EML may include as a dopant material, styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenamine (N-BDAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino)pyrene), etc.

The emission layer EML may further include a phosphorescence dopant material. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb) or thulium (Tm). For example, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), or platinum octaethyl porphyrin (PtOEP) may be used as the phosphorescence dopant. However, embodiments are not limited thereto.

The emission layer EML may include a quantum dot material. The core of the quantum dot may be selected from a II-VI group compound, a III-VI group compound, a Group I-III-VI compound, a Group III-V compound, a Group III-II-V compound, a IV-VI group compound, a IV group element, a IV group compound, and combinations thereof.

The II-VI group compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and mixtures thereof.

The III-V group compound may include a binary compound such as In₂S₃, and In₂Se₃, a ternary compound such as InGaS₃, and InGaSe₃, or optional combinations thereof.

The I-III-VI group compound may be selected from a ternary compound selected from the group consisting of AgInS, AgInS₂, CuInS, CuInS₂, AgGaS₂, CuGaS₂, CuGaO₂, AgGaO₂, AgAlO₂ and mixtures thereof, or a quaternary compound such as AgInGaS₂, and CuInGaS₂.

The III-V group compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. The III-V group compound may further include a II group metal. For example, InZnP, etc. may be selected as a III-II-V group compound.

The IV-VI group compound may be selected from the group consisting of a binary compound selected from the group consisting of SnS, SnSe, SnTe, PbS, PbSe, PbTe, and mixtures thereof; a ternary compound selected from the group consisting of SnSeS, SnSeTe, SnSTe, PbSeS, PbSeTe, PbSTe, SnPbS, SnPbSe, SnPbTe; and mixtures thereof, and a quaternary compound selected from the group consisting of SnPbSSe, SnPbSeTe, SnPbSTe, and mixtures thereof. The IV group element may be selected from the group consisting of Si, Ge, and a mixture thereof. The IV group compound may be a binary compound selected from the group consisting of SiC, SiGe, and a mixture thereof.

For example, a binary compound, a ternary compound, or a quaternary compound may be present at uniform concentration in a particle or may be present at a partially different concentration distribution state in the same particle. In an embodiment, a quantum dot may have a core/shell structure in which one quantum dot surrounds another quantum dot. The interface of the core and the shell may have a concentration gradient in which the concentration of an element present in the shell is decreased toward the center.

In embodiments, the quantum dot may have the above-described core-shell structure including a core including a nanocrystal and a shell wrapping the core. The shell of the quantum dot may function as a protection layer for preventing the chemical deformation of the core to maintain semiconductor properties and/or a charging layer for imparting the quantum dot with electrophoretic properties. The shell may have a single layer or a multilayer. The interface of the core and the shell may have a concentration gradient in which the concentration of an element present in the shell is decreased toward the center. Examples of the shell of the quantum dot may include a metal oxide, a non-metal oxide, a semiconductor compound, or combinations thereof.

For example, the metal or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, COO, Co₃O₄ and NiO, or a ternary compound such as MgAl₂O₄, CoFe₂O₄, NiFe₂O₄ and CoMn₂O₄, but embodiments are not limited thereto.

In an embodiment, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments are not limited thereto.

The quantum dot may have a full width of half maximum (FWHM) of emission wavelength spectrum equal to or less than about 45 nm. For example, the quantum dot may have a FWHM of a light emission wavelength spectrum equal to or less than about 40 nm. For example, the quantum dot may have a FWHM of a light emission wavelength spectrum equal to or less than about 30 nm. Within this range, color purity or color reproducibility may be improved. Light emitted via such quantum dot may be emitted in all directions, and light view angle properties may be improved.

The shape of the quantum dot may be selected from among generally used shapes in the art, without specific limitation. For example, the quantum dot may have a spherical, a pyramidal, a multi-arm, or a cubic shape, or the quantum dot may be in the form of a nanoparticle, a nanotube, a nanowire, a nanofiber, a nanoplate, etc.

The quantum dot may control the color of light emitted according to the particle size, and accordingly, the quantum dot may have various emission colors such as blue, red, and green.

In the luminescence devices ED of embodiments, shown in FIG. 3 to FIG. 6, the electron transport region ETR is provided on the emission layer EML. The electron transport region ETR may include at least one of an electron blocking layer HBL, an electron transport layer ETL, and an electron injection layer EIL. However, embodiments are not limited thereto.

The electron transport region ETR may have a single layer formed using a single material, a single layer formed using different materials, or a multilayer structure having multiple layers formed using different materials.

For example, the electron transport region ETR may have a single layer structure of an electron injection layer EIL or an electron transport layer ETL, or a single layer structure formed using an electron injection material and an electron transport material. The electron transport region ETR may have a single layer structure having different materials, or a structure stacked from the emission layer EML of electron transport layer ETL/electron injection layer EIL, or hole blocking layer HBL/electron transport layer ETL/electron injection layer EIL, without limitation. A thickness of the electron transport region ETR may be, for example, in a range of about 1,000 Å to about 1,500 Å.

The electron transport region ETR may be formed using various methods such as a vacuum deposition method, a spin coating method, a cast method, a Langmuir-Blodgett (LB) method, an inkjet printing method, a laser printing method, and a laser induced thermal imaging (LITI) method.

If the electron transport region ETR includes an electron transport layer ETL, the electron transport region ETR may include an anthracene-based compound. However, embodiments are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbnzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), and mixtures thereof, without limitation.

For example, the electron transport layer ETL may include a compound represented by Formula ET-1 below.

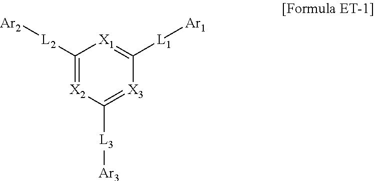

In Formula ET-1, at least one of X₁ to X₃ may be N, and the remainder of X₁ to X₃ may be C(Rₐ). Rₐ may be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ara may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-1, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms.

A thickness of the electron transport layer ETL may be in a range of about 100 Å to about 1,000 Å. For example, the thickness of the electron transport layer ETL may be in a range of about 150 Å to about 500 Å. If the thickness of the electron transport layer ETL satisfies the above-described range, satisfactory electron transport properties may be obtained without substantial increase of a driving voltage.

If the electron transport region ETR includes an electron injection layer EIL, the electron transport region ETR may include a metal halide such as LiF, NaCl, CsF, RbCl, RbI, CuI, and KI, a lanthanide metal such as Yb, or a co-depositing material of the metal halide and the lanthanide metal. For example, the electron transport region ETR may include KI:Yb, RbI:Yb, etc., as the co-depositing material. The electron transport region ETR may use a metal oxide such as Li₂O and BaO, or 8-hydroxy-lithium quinolate (Liq). However, embodiments are not limited thereto. The electron injection layer EIL may also be formed using a mixture material of an electron transport material and an insulating organo metal salt. The organo metal salt may be a material having an energy band gap of equal to or greater than about 4 eV. The organo metal salt may include, for example, metal acetates, metal benzoates, metal acetoacetates, metal acetylacetonates, or metal stearates. A thickness of the electron injection layer EIL may be in a range of about 1 Å to about 100 Å. For example, the thickness of the electron injection layer EIL may be in a range of about 3 Å to about 90 Å. If the thickness of the electron injection layer EIL satisfies the above described range, satisfactory electron injection properties may be obtained without inducing substantial increase of a driving voltage.

The electron transport region ETR may include a hole blocking layer HBL as described above. The hole blocking layer HBL may include, for example, at least one of 2,9-dimethyl-4,7-diphenyl-1,10-phenanthroline (BCP), and 4,7-diphenyl-1,10-phenanthroline (Bphen). However, embodiments are not limited thereto.

The second electrode EL2 is provided on the electron transport region ETR. The second electrode EL2 may be a common electrode. The second electrode EL2 may be a cathode or an anode, but embodiments are not limited thereto. For example, if the first electrode EL1 is an anode, the second cathode EL2 may be a cathode, and if the first electrode EL1 is a cathode, the second electrode EL2 may be an anode.

The second electrode EL2 may be a transmissive electrode, a transflective electrode, or a reflective electrode. If the second electrode EL2 is a transmissive electrode, the second electrode EL2 may include a transparent metal oxide, for example, ITO, IZO, ZnO, ITZO, etc.

If the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, compounds thereof, or mixtures thereof (for example, AgMg, AgYb, or MgAg). A multilayered structure including a reflective layer or a transflective layer formed using the above-described materials and a transparent conductive layer formed using ITO, IZO, ZnO, ITZO, etc. may be formed.

Though not shown, the second electrode EL2 may be electrically connected to an auxiliary electrode. If the second electrode EL2 is electrically connected to the auxiliary electrode, the resistance of the second electrode EL2 may decrease.

In an embodiment, a capping layer CPL may be further disposed on the second electrode EL2 of the luminescence device ED. The capping layer CPL may include a multilayer or a single layer.

In an embodiment, the capping layer CPL may include an organic layer or an inorganic layer. For example, if the capping layer CPL includes an inorganic material, the inorganic material may include an alkali metal compound such as LiF, an alkaline earth metal compound such as MgF₂, SiON, SiNx, SiOy, etc.

For example, if the capping layer CPL includes an organic material, the organic material may include α-NPD, NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol sol-9-yl) triphenylamine (TCTA), etc., or may include an epoxy resin, or acrylate such as methacrylate. The capping layer CPL may include at least one of Compounds P1 to P5 below, but embodiments are not limited thereto.

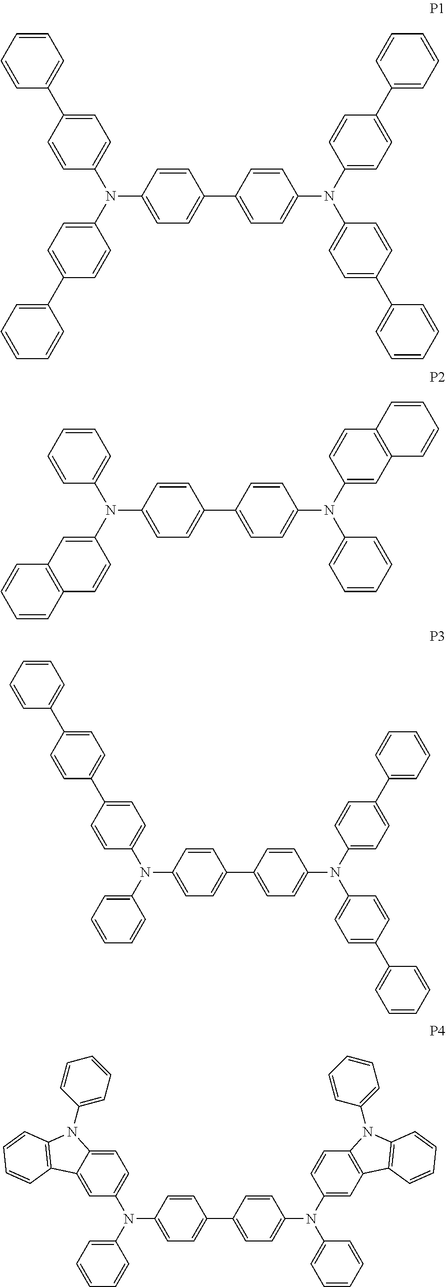 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

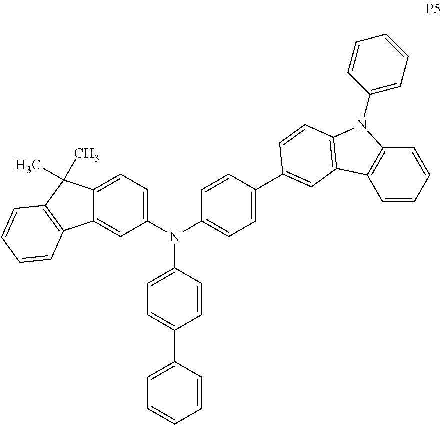 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

The refractive index of the capping layer CPL may be equal to or greater than about 1.6. For example, the refractive index of the capping layer CPL with respect to light in a wavelength range of about 550 nm to about 660 nm may be equal to or greater than about 1.6.

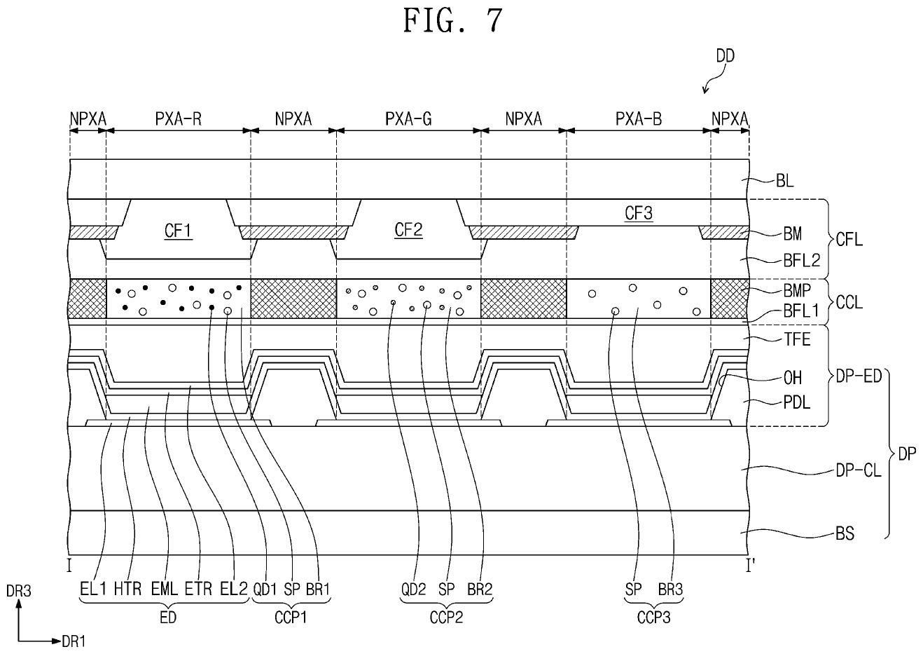

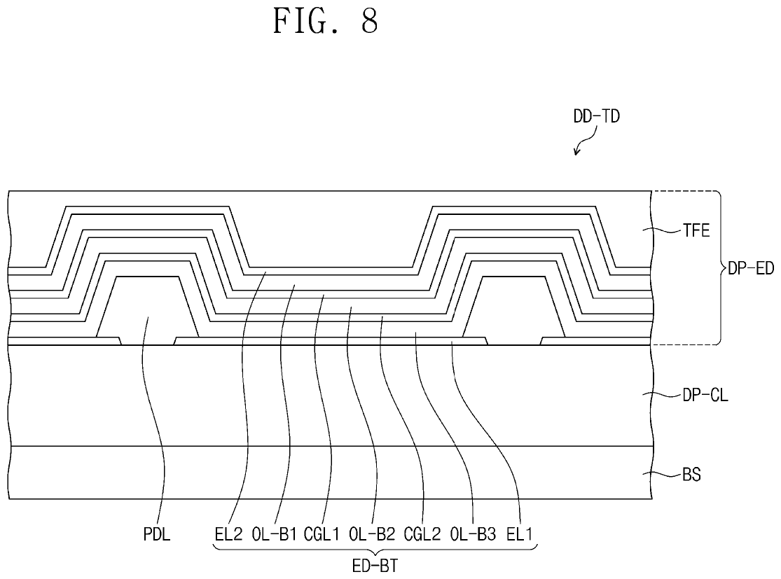

FIG. 7 and FIG. 8 are schematic cross-sectional views of display apparatuses according to embodiments. In the explanation on the display apparatuses of embodiments referring to FIG. 7 and FIG. 8, the overlapping parts with the explanation on FIG. 1 to FIG. 6 will not be explained again, and the different features will be explained chiefly.

Referring to FIG. 7, the display apparatus DD according to an embodiment may include a display panel DP including a display device layer DP-ED, a light controlling layer CCL disposed on the display panel DP, and a color filter layer CFL.

In an embodiment shown in FIG. 7, the display panel DP includes a base layer BS, a circuit layer DP-CL provided on the base layer BS, and a display device layer DP-ED, and the display device layer DP-ED may include a luminescence device ED.

The luminescence device ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. The same structures of the luminescence devices of FIG. 3 to FIG. 6 may be applied to the structure of the luminescence device ED shown in FIG. 7.

Referring to FIG. 7, the emission layer EML may be disposed in an opening part OH defined in a pixel definition layer PDL. For example, the emission layer EML divided by the pixel definition layer PDL and correspondingly provided to each of luminous areas PXA-R, PXA-G, and PXA-B may emit light in a same wavelength region. In the display apparatus DD of an embodiment, the emission layer EML may emit blue light. In contrast to the drawings, in an embodiment, the emission layer EML may be provided as a common layer for all luminous areas PXA-R, PXA-G, and PXA-B.

The light controlling layer CCL may be disposed on the display panel DP. The light controlling layer CCL may include a light converter. The light converter may include a quantum dot or a phosphor. The light converter may convert the wavelength of light provided and then emit converted light. For example, the light controlling layer CCL may be a layer including a quantum dot or a layer including a phosphor.

The light controlling layer CCL may include multiple light controlling parts CCP1, CCP2, and CCP3. The light controlling parts CCP1, CCP2, and CCP3 may be separated from one another.

Referring to FIG. 7, a partition pattern BMP may be disposed between the separated light controlling parts CCP1, CCP2, and CCP3, but embodiments are not limited thereto. In FIG. 7, the partition pattern BMP is shown not to be overlapped with the light controlling parts CCP1, CCP2, and CCP3, but in an embodiment, at least a portion of the edge of the light controlling parts CCP1, CCP2, and CCP3 may be overlapped with the partition pattern BMP.

The light controlling layer CCL may include a first light controlling part CCP1 including a first quantum dot QD1 converting first color light provided from the luminescence device ED into second color light, a second light controlling part CCP2 including a second quantum dot QD2 converting first color light into third color light, and a third light controlling part CCP3 transmitting first color light.

In an embodiment, the first light controlling part CCP1 provides red light which is the second color light, and the second light controlling part CCP2 may provide green light which is the third color light. The third color controlling part CCP3 may transmit and provide blue light which is the first color light provided from the luminescence device ED. For example, the first quantum dot QD1 may be a red quantum dot, and the second quantum dot QD2 may be a green quantum dot. For the quantum dots QD1 and QD2, the same explanation as described above may be applied.

The light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include a quantum dot but include the scatterer SP.

The scatterer SP may be an inorganic particle. For example, the scatterer SP may include at least one of TiO₂, ZnO, Al₂O₃, SiO₂, and hollow silica. The scatterer SP may include at least one of TiO₂, ZnO, Al₂O₃, SiO₂, and hollow silica, or may be a mixture of two or more materials selected from TiO₂, ZnO, Al₂O₃, SiO₂, and hollow silica.

The first light controlling part CCP1, the second light controlling part CCP2, and the third light controlling part CCP3 may respectively include base resins BR1, BR2, and BR3 in which the quantum dots QD1 and QD2 and the scatterer SP are dispersed. In an embodiment, the first light controlling part CCP1 may include the first quantum dot QD1 and the scatterer SP dispersed in a first base resin BR1, the second light controlling part CCP2 may include the second quantum dot QD2 and the scatterer SP dispersed in a second base resin BR2, and the third light controlling part CCP3 may include the scatterer SP dispersed in a third base resin BR3. The base resins BR1, BR2, and BR3 are media in which the quantum dots QD1 and QD2 and the scatterer SP are dispersed, and may be formed of various resin compositions, which may be generally referred to as a binder. The base resins BR1, BR2, and BR3 may be transparent resins. In an embodiment, the first base resin BR1, the second base resin BR2, and the third base resin BR3 each may be the same as or different from each other.

The light controlling layer CCL may include a barrier layer BFL1. The barrier layer BFL1 may block the penetration of moisture and/or oxygen (hereinafter, will be referred to as “humidity/oxygen”). The barrier layer BFL1 may be disposed on the light controlling parts CCP1, CCP2, and CCP3 to block the exposure of the light controlling parts CCP1, CCP2, and CCP3 to humidity/oxygen. The barrier layer BFL1 may cover the light controlling parts CCP1, CCP2, and CCP3. A barrier layer BFL2 may be provided between the light controlling parts CCP1, CCP2, and CCP3 and a color filter layer CFL.

Barrier layers BFL1 and BFL2 may include at least one inorganic layer. For example, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide and silicon oxynitride, or a metal thin film securing light transmittance. The barrier layers BFL1 and BFL2 may further include an organic layer. The barrier layers BFL1 and BFL2 may be composed of a single layer or of multiple layers.

In the display apparatus DD of an embodiment, the color filter layer CFL may be disposed on the light controlling layer CCL. For example, the color filter layer CFL may be disposed directly on the light controlling layer CCL. In an embodiment, the barrier layer BFL2 may be omitted.

The color filter layer CFL may include a light blocking part BM and filters CF1, CF2, and CF3. The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. Each of the filters CF1, CF2, and CF3 may include a polymer photosensitive resin and a pigment or dye. The first filter CF1 may include a red pigment or dye, the second filter CF2 may include a green pigment or dye, and the third filter CF3 may include a blue pigment or dye. However, embodiments are not limited thereto, and the third filter CF3 may not include a pigment or dye. The third filter CF3 may include a polymer photosensitive resin and not include a pigment or dye. The third filter CF3 may be transparent. The third filter CF3 may be formed using a transparent photosensitive resin.

In an embodiment, the first filter CF1 and the second filter CF2 may be yellow filters. The first filter CF1 and the second filter CF2 may be provided in one body without distinction.

The light blocking part BM may be a black matrix. The light blocking part BM may be formed by including an organic light blocking material or an inorganic light blocking material including a black pigment or black dye. The light blocking part BM may prevent light leakage phenomenon and divide the boundaries among adjacent filters CF1, CF2, and CF3. In an embodiment, the light blocking part BM may be formed as a blue filter.

Each of the first to third filters CF1, CF2, and CF3 may be disposed corresponding to each of a red light-emitting area PXA-R, green light-emitting area PXA-G, and blue light-emitting area PXA-B, respectively.

On the color filter layer CFL, a base substrate BL may be disposed. The base substrate BL may be a member providing a base surface on which the color filter layer CFL, the light controlling layer CCL, etc. are disposed. The base substrate BL may be a glass substrate, a metal substrate, a plastic substrate, etc. However, embodiments are not limited thereto, and the base substrate BL may be an inorganic layer, an organic layer, or a composite material layer. In contrast to the drawing, in another embodiment, the base substrate BL of an embodiment may be omitted.

FIG. 8 is a schematic cross-sectional view showing a portion of the display apparatus according to an embodiment. In FIG. 8, the schematic cross-sectional view of a portion corresponding to the display panel DP in FIG. 7 is shown. In a display apparatus DD-TD of an embodiment, a luminescence device ED-BT may include luminous structures OL-B1, OL-B2, and OL-B3. The luminescence device ED-BT may include oppositely disposed first electrode EL1 and second electrode EL2, and the luminous structures OL-B1, OL-B2, and OL-B3 stacked in order in a thickness direction and provided between the first electrode EL1 and the second electrode EL2. Each of the luminous structures OL-B1, OL-B2, and OL-B3 may include an emission layer EML (FIG. 7), and a hole transport region HTR and an electron transport region ETR disposed with the emission layer EML (FIG. 7) therebetween.

For example, the luminescence device ED-BT included in the display apparatus DD-TD of an embodiment may be a luminescence device of a tandem structure including multiple emission layers.

In an embodiment shown in FIG. 8, light emitted from the luminous structures OL-B1, OL-B2, and OL-B3 may be all blue light. However, embodiments are not limited thereto, and the wavelength regions of light emitted from the luminous structures OL-B1, OL-B2, and OL-B3 may be different from each other. For example, the luminescence device ED-BT including the multiple luminous structures OL-B1, OL-B2, and OL-B3 emitting light in different wavelength regions may emit white light.

A charge generating layer CGL1 and CGL2 may be disposed between neighboring luminous structures OL-B1, OL-B2, and OL-B3. The charge generating layer CGL1 and CGL2 may include a p-type charge generating layer and/or an n-type charge generating layer.

Hereinafter, the disclosure will be explained referring to embodiments and comparative embodiments. The following embodiments are only illustrations to assist the understanding of the disclosure, and the scope of the disclosure is not limited thereto.

### Synthesis Examples

The amine compound according to an embodiment may be synthesized, for example, as follows. However, the synthesis method of the amine compound according to an embodiment is not limited thereto.

1. Synthesis of Compound A2

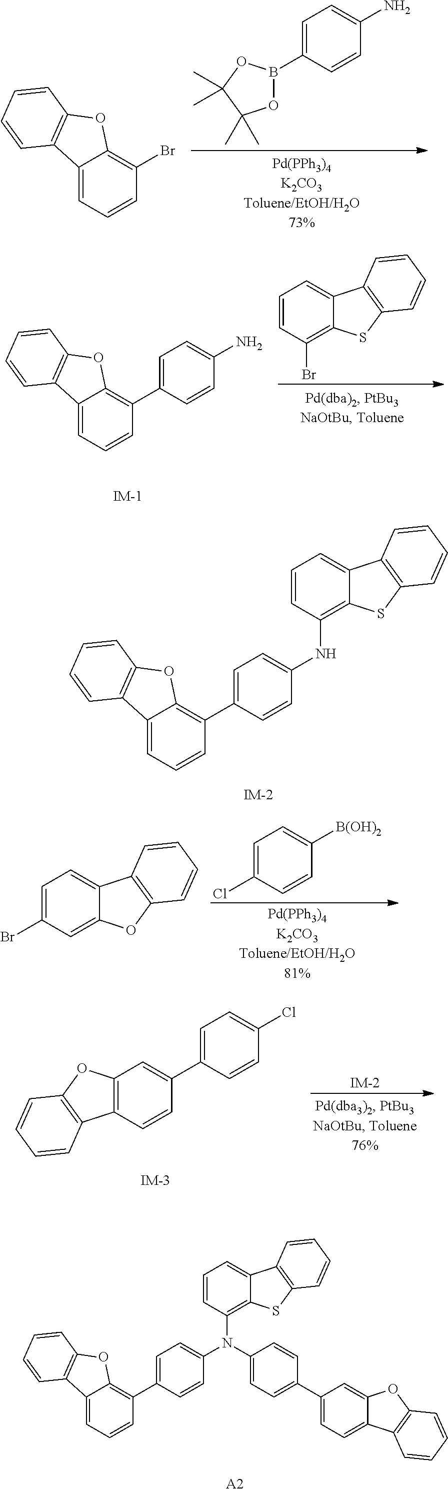

(1) Synthesis of Intermediate IM-1

Under an Ar atmosphere, to a 1,000 mL, three-neck flask, 20.00 g (80.9 mmol) of 4-bromodibenzofuran, 19.51 g (1.1 equiv, 89.0 mmol) of 4-(4,4,5,5-tetramethyl-1,3,2-dioxaborolan-2-yl)aniline, 33.56 g (3.0 equiv, 242.8 mmol) of K₂CO₃, 4.68 g (0.05 equiv, 4.1 mmol) of Pd(PPh₃)₄, and 567 mL of a mixture solution of toluene/EtOH/H₂O (4/2/1) were added in order and heated and stirred at about 80° C. After cooling to room temperature, the reaction solution was extracted with toluene. An aqueous layer was removed, and an organic layer was washed with a saturated saline solution and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-1 (16.58 g, yield 79%).

FAB-MS measurement was conducted, and Intermediate IM-1 was identified by observing a mass number of m/z=259 as a molecular ion peak.

(2) Synthesis of Intermediate IM-2

Under an Ar atmosphere, to a 500 ml, three-neck flask, 10.00 g (38.6 mmol) of IM-1, 0.67 g (0.03 equiv, 1.2 mmol) of Pd(dba)₂, 3.71 g (1.0 equiv, 38.6 mmol) of NaOtBu, 193 mL of toluene, 11.16 g (1.1 equiv, 42.4 mmol) of 4-bromodibenzothiophene and 0.78 g (0.1 equiv, 3.9 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-2 (12.77 g, yield 75%).

FAB-MS measurement was conducted, and Intermediate IM-2 was identified by observing a mass number of m/z=441 as a molecular ion peak.

(3) Synthesis of Intermediate IM-3

Under an Ar atmosphere, to a 1,000 mL, three-neck flask, 20.00 g (80.9 mmol) of 3-bromodibenzofuran, 13.92 g (1.1 equiv, 89.0 mmol) of 4-chlorophenylboronic acid, 33.56 g (3.0 equiv, 242.8 mmol) of K₂CO₃, 4.68 g (0.05 equiv, 4.1 mmol) of Pd(PPh₃)₄, and 567 mL of a mixture solution of toluene/EtOH/H₂O (4/2/1) were added in order and heated and stirred at about 80° C. After cooling to room temperature, the reaction solution was extracted with toluene. An aqueous layer was removed, and an organic layer was washed with a saturated saline solution and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-3 (18.28 g, yield 81%).

FAB-MS measurement was conducted, and Intermediate IM-3 was identified by observing a mass number of m/z=278 as a molecular ion peak.

(4) Synthesis of Compound A2

Under an Ar atmosphere, to a 300 ml, three-neck flask, 10.00 g (22.6 mmol) of IM-2, 0.39 g (0.03 equiv, 0.7 mmol) of Pd(dba)₂, 4.35 g (2.0 equiv, 45.3 mmol) of NaOtBu, 113 mL of toluene, 6.94 g (1.1 equiv, 24.9 mmol) of IM-3 and 0.46 g (0.1 equiv, 2.3 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Compound A2 (11.77 g, yield 76%) as a solid.

FAB-MS measurement was conducted, and Compound A2 was identified by observing a mass number of m/z=683 as a molecular ion peak.

2. Synthesis of Compound A58

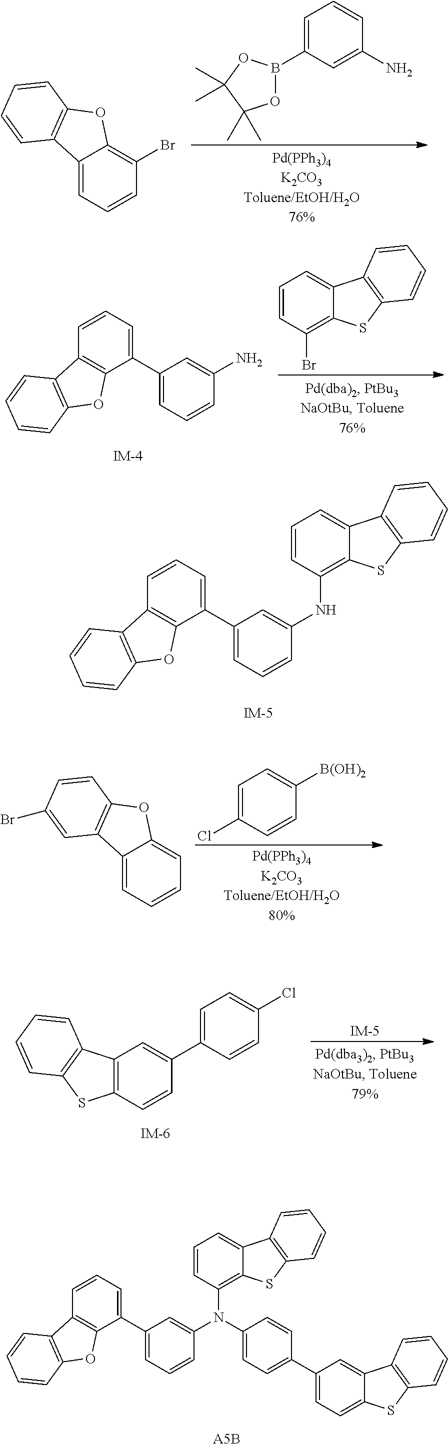

(1) Synthesis of Intermediate IM-4

Under an Ar atmosphere, to a 1,000 mL, three-neck flask, 20.00 g (80.9 mmol) of 4-bromodibenzofuran, 19.51 g (1.1 equiv, 89.0 mmol) of 3-(4,4,5,5-tetramethyl-1,3,2-dioxaborolan-2-yl)aniline, 33.56 g (3.0 equiv, 242.8 mmol) of K₂CO₃, 4.68 g (0.05 equiv, 4.1 mmol) of Pd(PPh₃)₄, and 567 mL of a mixture solution of toluene/EtOH/H₂O (4/2/1) were added in order and heated and stirred at about 80° C. After cooling to room temperature, the reaction solution was extracted with toluene. An aqueous layer was removed, and an organic layer was washed with a saturated saline solution and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-4 (15.95 g, yield 76%).

FAB-MS measurement was conducted, and Intermediate IM-4 was identified by observing a mass number of m/z=259 as a molecular ion peak.

(2) Synthesis of Intermediate IM-5

Under an Ar atmosphere, to a 500 ml, three-neck flask, 10.00 g (38.6 mmol) of IM-4, 0.67 g (0.03 equiv, 1.2 mmol) of Pd(dba)₂, 3.71 g (1.0 equiv, 38.6 mmol) of NaOtBu, 193 mL of toluene, 11.16 g (1.1 equiv, 42.4 mmol) of 4-bromodibenzothiophene and 0.78 g (0.1 equiv, 3.9 mmol) of tBu₃P were added in order, and heated, refluxed, and stirred.

After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-5 (12.94 g, yield 76%).

FAB-MS measurement was conducted, and Intermediate IM-5 was identified by observing a mass number of m/z=441 as a molecular ion peak.

(3) Synthesis of Intermediate IM-6

Under an Ar atmosphere, to a 500 mL, three-neck flask, 20.00 g (76.0 mmol) of 2-bromodibenzothiophene, 13.07 g (1.1 equiv, 83.6 mmol) of 4-chlorophenylboronic acid, 31.51 g (3.0 equiv, 228.0 mmol) of K₂CO₃, 4.39 g (0.05 equiv, 3.8 mmol) of Pd(PPh₃)₄, and 532 mL of a mixture solution of toluene/EtOH/H₂O (4/2/1) were added in order and heated and stirred at about 80° C. After cooling to room temperature, the reaction solution was extracted with toluene. An aqueous layer was removed, and an organic layer was washed with a saturated saline solution and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-6 (17.92 g, yield 80%).

FAB-MS measurement was conducted, and Intermediate IM-6 was identified by observing a mass number of m/z=294 as a molecular ion peak.

(4) Synthesis of Compound A58

Under an Ar atmosphere, to a 300 ml, three-neck flask, 10.00 g (22.6 mmol) of IM-5, 0.39 g (0.03 equiv, 0.7 mmol) of Pd(dba)₂, 4.35 g (2.0 equiv, 45.3 mmol) of NaOtBu, 113 mL of toluene, 7.34 g (1.1 equiv, 24.9 mmol) of IM-6 and 0.46 g (0.1 equiv, 2.3 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Compound A58 (12.52 g, yield 79%) as a solid.

FAB-MS measurement was conducted, and Compound A58 was identified by observing a mass number of m/z=699 as a molecular ion peak.

3. Synthesis of Compound B15

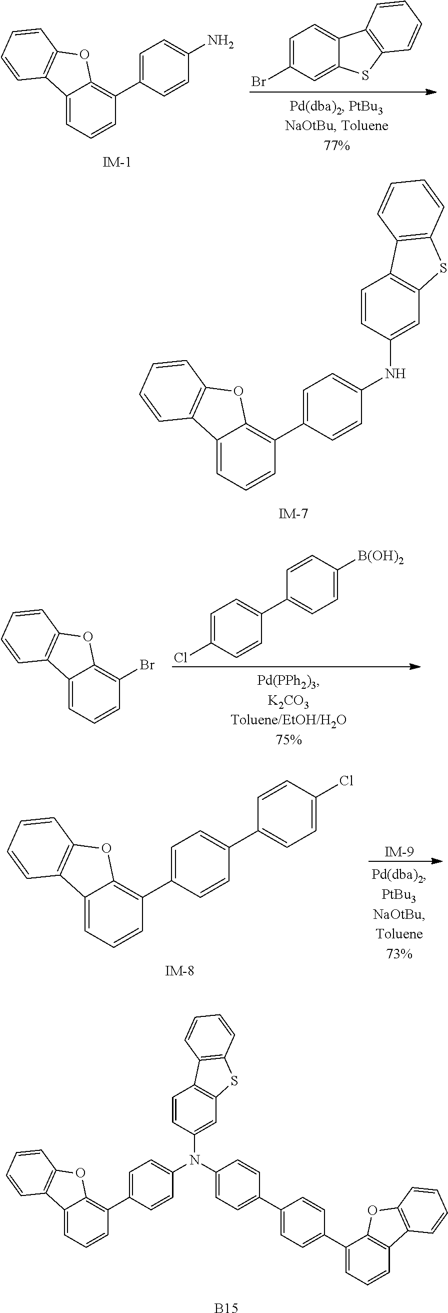

(1) Synthesis of Intermediate IM-7

Under an Ar atmosphere, to a 500 ml, three-neck flask, 10.00 g (38.6 mmol) of IM-1, 0.67 g (0.03 equiv, 1.2 mmol) of Pd(dba)₂, 3.71 g (1.0 equiv, 38.6 mmol) of NaOtBu, 193 mL of toluene, 11.16 g (1.1 equiv, 42.4 mmol) of 3-bromodibenzothiophene and 0.78 g (0.1 equiv, 3.9 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-7 (13.11 g, yield 77%).

FAB-MS measurement was conducted, and Intermediate IM-7 was identified by observing a mass number of m/z=441 as a molecular ion peak.

(2) Synthesis of Intermediate IM-8

Under an Ar atmosphere, to a 1,000 mL, three-neck flask, 20.00 g (80.9 mmol) of 4-bromodibenzofuran, 20.70 g (1.1 equiv, 89.0 mmol) of [4′-chloro-(1,1′-biphenyl)-4-yl]boronic acid, 33.56 g (3.0 equiv, 242.8 mmol) of K₂CO₃, 4.68 g (0.05 equiv, 4.1 mmol) of Pd(PPh₃)₄, and 567 mL of a mixture solution of toluene/EtOH/H₂O (4/2/1) were added in order and heated and stirred at about 80° C. After cooling to room temperature, the reaction solution was extracted with toluene. An aqueous layer was removed, and an organic layer was washed with a saturated saline solution and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-8 (17.90 g, yield 75%).

FAB-MS measurement was conducted, and Intermediate IM-8 was identified by observing a mass number of m/z=354 as a molecular ion peak.

(3) Synthesis of Compound B15

Under an Ar atmosphere, to a 300 ml, three-neck flask, 10.00 g (22.6 mmol) of IM-7, 0.39 g (0.03 equiv, 0.7 mmol) of Pd(dba)₂, 4.35 g (2.0 equiv, 45.3 mmol) of NaOtBu, 113 mL of toluene, 8.84 g (1.1 equiv, 24.9 mmol) of IM-8 and 0.46 g (0.1 equiv, 2.3 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Compound B15 (12.56 g, yield 73%) as a solid.

FAB-MS measurement was conducted, and Compound B15 was identified by observing a mass number of m/z=759 as a molecular ion peak.

4. Synthesis of Compound B40

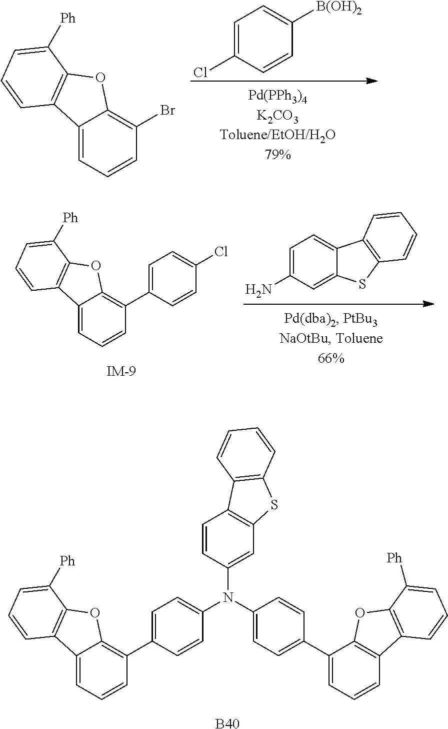

(1) Synthesis of Intermediate IM-9

Under an Ar atmosphere, to a 1,000 mL, three-neck flask, 20.00 g (61.9 mmol) of 4-bromo-6-phenyldibenzofuran, 10.64 g (1.1 equiv, 68.1 mmol) of 4-chlorophenylboronic acid, 25.7 g (3.0 equiv, 185.6 mmol) of K₂CO₃, 3.58 g (0.05 equiv, 3.1 mmol) of Pd(PPh₃)₄, and 433 mL of a mixture solution of toluene/EtOH/H₂O (4/2/1) were added in order and heated and stirred at about 80° C. After cooling to room temperature, the reaction solution was extracted with toluene. An aqueous layer was removed, and an organic layer was washed with a saturated saline solution and dried with MgSO₄.

MgSO₄ was filtered and separated, and an organic layer was concentrated. The crude product thus obtained was separated by silica gel column chromatography (using a mixture solvent of hexane and toluene as a developing layer) to obtain Intermediate IM-9 (17.35 g, yield 79%).

FAB-MS measurement was conducted, and Intermediate IM-9 was identified by observing a mass number of m/z=354 as a molecular ion peak.

(2) Synthesis of Compound B40

Under an Ar atmosphere, to a 300 ml, three-neck flask, 4.00 g (20.7 mmol) of 3-aminodibenzothiophene, 0.69 g (0.06 equiv, 1.2 mmol) of Pd(dba)₂, 7.72 g (4.0 equiv, 80.3 mmol) of NaOtBu, 100 mL of toluene, 15.67 g (2.2 equiv, 44.2 mmol) of IM-9 and 0.81 g (0.2 equiv, 4.0 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Compound B40 (11.41 g, yield 68%) as a solid.

FAB-MS measurement was conducted, and Compound B40 was identified by observing a mass number of m/z=836 as a molecular ion peak.

5. Synthesis of Compound C47

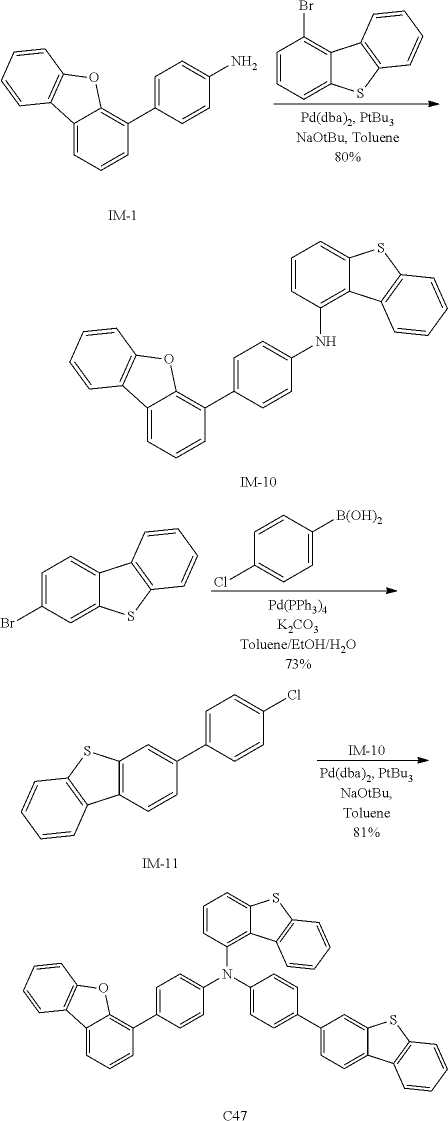

(1) Synthesis of Intermediate IM-10

Under an Ar atmosphere, to a 500 ml, three-neck flask, 10.00 g (38.6 mmol) of IM-1, 0.67 g (0.03 equiv, 1.2 mmol) of Pd(dba)₂, 3.71 g (1.0 equiv, 38.6 mmol) of NaOtBu, 193 mL of toluene, 11.16 g (1.1 equiv, 42.4 mmol) of 1-bromodibenzothiophene and 0.78 g (0.1 equiv, 3.9 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-10 (13.62 g, yield 80%).

FAB-MS measurement was conducted, and Intermediate IM-10 was identified by observing a mass number of m/z=441 as a molecular ion peak.

(2) Synthesis of Intermediate IM-11

Under an Ar atmosphere, to a 1,000 mL, three-neck flask, 20.00 g (76.0 mmol) of 3-bromodibenzothiophene, 13.07 g (1.1 equiv, 83.6 mmol) of 4-chlorophenylboronic acid, 31.51 g (3.0 equiv, 228.0 mmol) of K₂CO₃, 4.39 g (0.05 equiv, 3.8 mmol) of Pd(PPh₃)₄, and 532 mL of a mixture solution of toluene/EtOH/H₂O (4/2/1) were added in order and heated and stirred at about 80° C. After cooling to room temperature, the reaction solution was extracted with toluene. An aqueous layer was removed, and an organic layer was washed with a saturated saline solution and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-11 (16.36 g, yield 73%).

FAB-MS measurement was conducted, and Intermediate IM-11 was identified by observing a mass number of m/z=294 as a molecular ion peak.

(3) Synthesis of Compound C47

Under an Ar atmosphere, to a 300 ml, three-neck flask, 10.00 g (22.6 mmol) of IM-10, 0.39 g (0.03 equiv, 0.7 mmol) of Pd(dba)₂, 4.35 g (2.0 equiv, 45.3 mmol) of NaOtBu, 113 mL of toluene, 7.34 g (1.1 equiv, 24.9 mmol) of IM-11 and 0.46 g (0.1 equiv, 2.3 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Compound C47 (12.84 g, yield 81%) as a solid.

FAB-MS measurement was conducted, and Compound C47 was identified by observing a mass number of m/z=699 as a molecular ion peak.

6. Synthesis of Compound C70

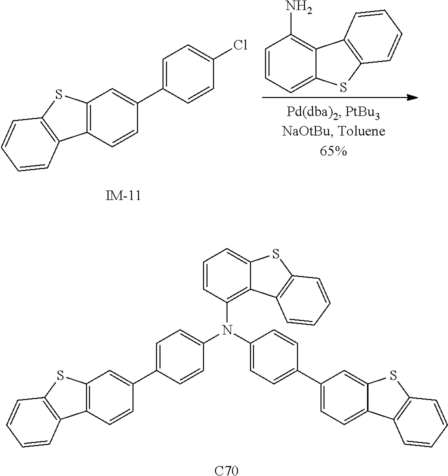

(1) Synthesis of Compound C70

Under an Ar atmosphere, to a 300 ml, three-neck flask, 4.00 g (20.7 mmol) of 1-aminodibenzothiophene, 0.69 g (0.06 equiv, 1.2 mmol) of Pd(dba)₂, 7.72 g (4.0 equiv, 80.3 mmol) of NaOtBu, 100 mL of toluene, 13.02 g (2.2 equiv, 44.2 mmol) of IM-11 and 0.81 g (0.2 equiv, 4.0 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Compound C70 (9.34 g, yield 65%) as a solid.

FAB-MS measurement was conducted, and Compound C70 was identified by observing a mass number of m/z=715 as a molecular ion peak.

7. Synthesis of Compound D1

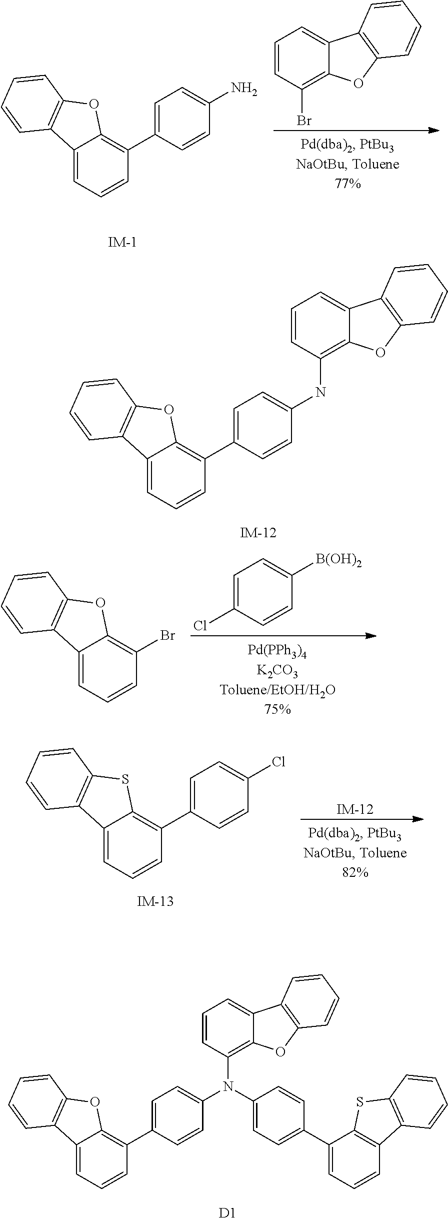

(1) Synthesis of Intermediate IM-12

Under an Ar atmosphere, to a 500 ml, three-neck flask, 10.00 g (38.6 mmol) of IM-1, 0.67 g (0.03 equiv, 1.2 mmol) of Pd(dba)₂, 3.71 g (1.0 equiv, 38.6 mmol) of NaOtBu, 193 mL of toluene, 10.48 g (1.1 equiv, 42.4 mmol) of 4-bromodibenzofuran and 0.78 g (0.1 equiv, 3.9 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-12 (12.63 g, yield 77%).

FAB-MS measurement was conducted, and Intermediate IM-12 was identified by observing a mass number of m/z=425 as a molecular ion peak.

(2) Synthesis of Intermediate IM-13

Under an Ar atmosphere, to a 1,000 mL, three-neck flask, 20.00 g (76.0 mmol) of 4-bromodibenzothiophene, 13.07 g (1.1 equiv, 83.6 mmol) of 4-chlorophenylboronic acid, 31.51 g (3.0 equiv, 228.0 mmol) of K₂CO₃, 4.39 g (0.05 equiv, 3.8 mmol) of Pd(PPh₃)₄, and 532 mL of a mixture solution of toluene/EtOH/H₂O (4/2/1) were added in order and heated and stirred at about 80° C. After cooling to room temperature, the reaction solution was extracted with toluene. An aqueous layer was removed, and an organic layer was washed with a saturated saline solution and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-13 (17.70 g, yield 79%).

FAB-MS measurement was conducted, and Intermediate IM-13 was identified by observing a mass number of m/z=294 as a molecular ion peak.

(3) Synthesis of Compound D1

Under an Ar atmosphere, to a 300 ml, three-neck flask, 10.00 g (23.5 mmol) of IM-12, 0.41 g (0.03 equiv, 0.7 mmol) of Pd(dba)₂, 4.52 g (2.0 equiv, 47.0 mmol) of NaOtBu, 113 mL of toluene, 7.62 g (1.1 equiv, 25.9 mmol) of IM-13 and 0.48 g (0.1 equiv, 2.4 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Compound D1 (13.18 g, yield 82%) as a solid.

FAB-MS measurement was conducted, and Compound D1 was identified by observing a mass number of m/z=683 as a molecular ion peak.

8. Synthesis of Compound D44

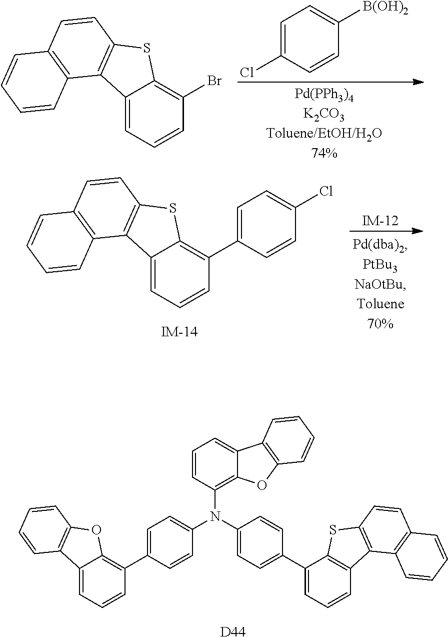

(1) Synthesis of Intermediate IM-14

Under an Ar atmosphere, to a 1,000 ml, three-neck flask, 20.00 g (63.9 mmol) of 8-bromodibenzo[b]naphtho[1,2-d]thiophene, 10.98 g (1.1 equiv, 70.2 mmol) of 4-chlorophenylboronic acid, 26.48 g (3.0 equiv, 191.6 mmol) of K₂CO₃, 3.69 g (0.05 equiv, 3.2 mmol) of Pd(PPh₃)₄, and 447 mL of a mixture solution of toluene/EtOH/H₂O (4/2/1) were added in order and heated and stirred at about 80° C. After cooling to room temperature, the reaction solution was extracted with toluene. An aqueous layer was removed, and an organic layer was washed with a saturated saline solution and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-14 (16.30 g, yield 74%).

FAB-MS measurement was conducted, and Intermediate IM-14 was identified by observing a mass number of m/z=344 as a molecular ion peak.

(2) Synthesis of Compound D44

Under an Ar atmosphere, to a 300 ml, three-neck flask, 10.00 g (23.5 mmol) of IM-12, 0.41 g (0.03 equiv, 0.7 mmol) of Pd(dba)₂, 4.52 g (2.0 equiv, 47.0 mmol) of NaOtBu, 113 mL of toluene, 8.92 g (1.1 equiv, 25.9 mmol) of IM-14 and 0.48 g (0.1 equiv, 2.4 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Compound D44 (12.07 g, yield 70%) as a solid.

FAB-MS measurement was conducted, and Compound D44 was identified by observing a mass number of m/z=733 as a molecular ion peak.

9. Synthesis of Compound E18

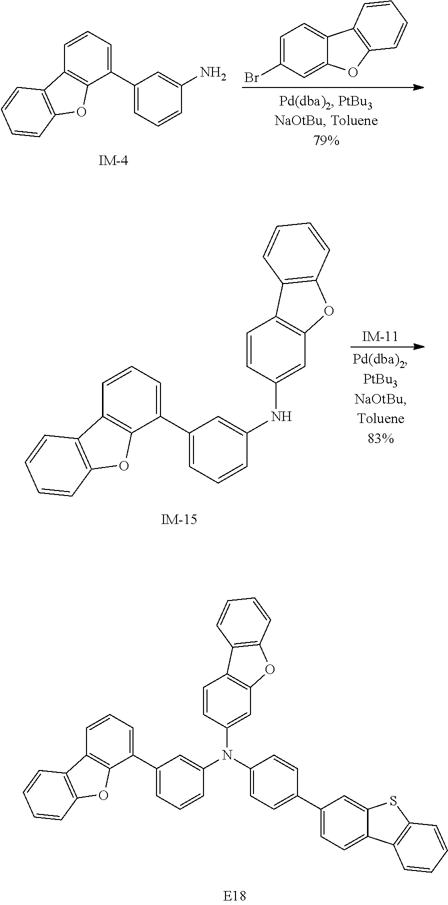

(1) Synthesis of Intermediate IM-15

Under an Ar atmosphere, to a 500 ml, three-neck flask, 10.00 g (38.6 mmol) of IM-4, 0.67 g (0.03 equiv, 1.2 mmol) of Pd(dba)₂, 3.71 g (1.0 equiv, 38.6 mmol) of NaOtBu, 193 mL of toluene, 10.48 g (1.1 equiv, 42.4 mmol) of 3-bromodibenzofuran and 0.78 g (0.1 equiv, 3.9 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-15 (12.96 g, yield 79%).

FAB-MS measurement was conducted, and Intermediate IM-15 was identified by observing a mass number of m/z=425 as a molecular ion peak.

(2) Synthesis of Compound E18

Under an Ar atmosphere, to a 300 ml, three-neck flask, 10.00 g (23.5 mmol) of IM-15, 0.41 g (0.03 equiv, 0.7 mmol) of Pd(dba)₂, 4.52 g (2.0 equiv, 47.0 mmol) of NaOtBu, 113 mL of toluene, 7.62 g (1.1 equiv, 25.9 mmol) of IM-11 and 0.48 g (0.1 equiv, 2.4 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Compound E18 (13.34 g, yield 83%) as a solid.

FAB-MS measurement was conducted, and Compound E18 was identified by observing a mass number of m/z=683 as a molecular ion peak.

10. Synthesis of Compound E35

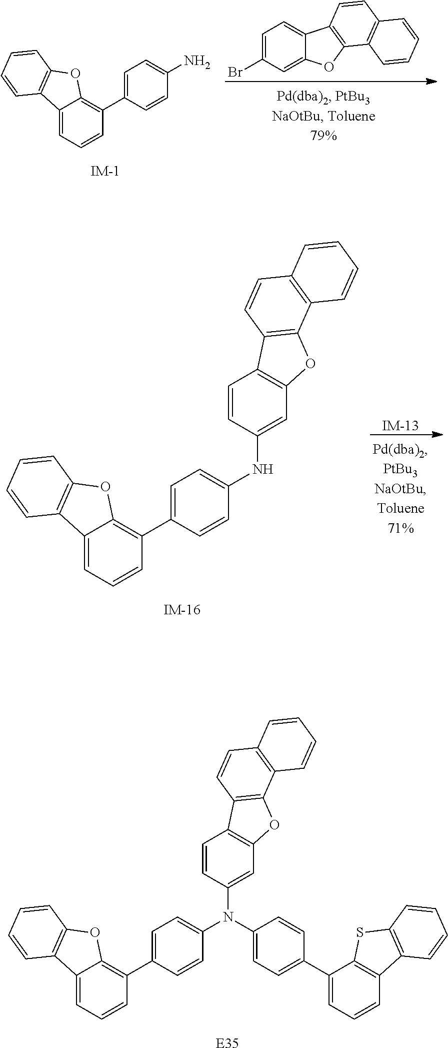

(1) Synthesis of Intermediate IM-16

Under an Ar atmosphere, to a 500 ml, three-neck flask, 10.00 g (38.6 mmol) of IM-1, 0.67 g (0.03 equiv, 1.2 mmol) of Pd(dba)₂, 3.71 g (1.0 equiv, 38.6 mmol) of NaOtBu, 193 mL of toluene, 12.61 g (1.1 equiv, 42.4 mmol) of 9-bromonaphtho[1,2-b]benzofuran and 0.78 g (0.1 equiv, 3.9 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-16 (14.49 g, yield 79%).

FAB-MS measurement was conducted, and Intermediate IM-16 was identified by observing a mass number of m/z=475 as a molecular ion peak.

(2) Synthesis of Compound E35

Under an Ar atmosphere, to a 300 ml, three-neck flask, 10.00 g (21.0 mmol) of IM-16, 0.36 g (0.03 equiv, 0.6 mmol) of Pd(dba)₂, 4.04 g (2.0 equiv, 42.1 mmol) of NaOtBu, 105 mL of toluene, 6.82 g (1.1 equiv, 23.1 mmol) of IM-13 and 0.43 g (0.1 equiv, 2.1 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Compound E35 (10.96 g, yield 71%) as a solid.

FAB-MS measurement was conducted, and Compound E35 was identified by observing a mass number of m/z=733 as a molecular ion peak.

11. Synthesis of Compound F9

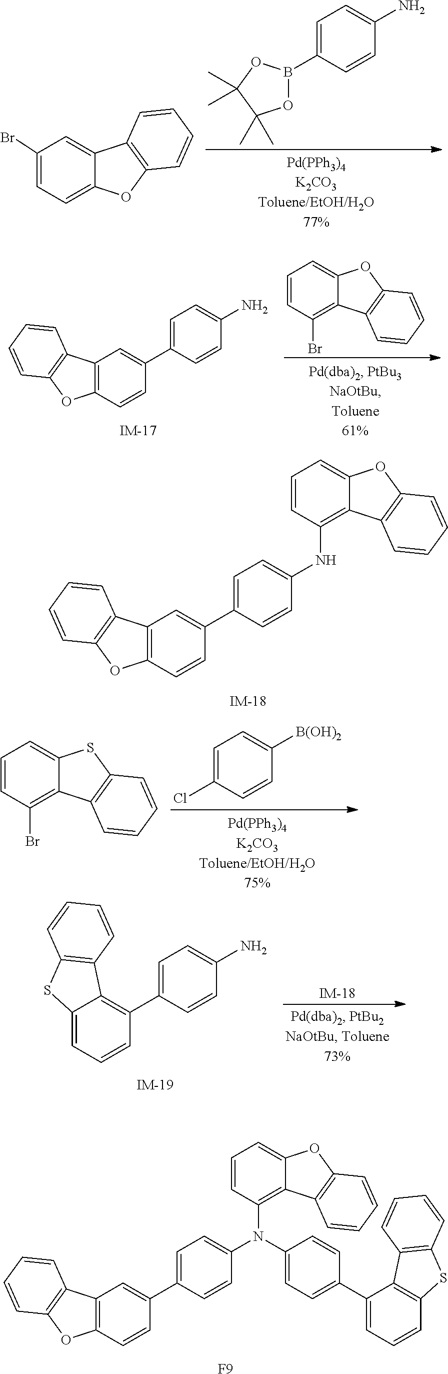

(1) Synthesis of Intermediate IM-17

Under an Ar atmosphere, to a 1,000 mL, three-neck flask, 20.00 g (80.9 mmol) of 4-bromodibenzofuran, 19.51 g (1.1 equiv, 89.0 mmol) of 4-(4,4,5,5-tetramethyl-1,3,2-dioxaborolan-2-yl)aniline, 33.56 g (3.0 equiv, 242.8 mmol) of K₂CO₃, 4.68 g (0.05 equiv, 4.1 mmol) of Pd(PPh₃)₄, and 567 mL of a mixture solution of toluene/EtOH/H₂O (4/2/1) were added in order and heated and stirred at about 80° C. After cooling to room temperature, the reaction solution was extracted with toluene. An aqueous layer was removed, and an organic layer was washed with a saturated saline solution and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-17 (16.16 g, yield 77%).

FAB-MS measurement was conducted, and Intermediate IM-17 was identified by observing a mass number of m/z=259 as a molecular ion peak.

(2) Synthesis of Intermediate IM-18

Under an Ar atmosphere, to a 500 ml, three-neck flask, 10.00 g (38.6 mmol) of IM-17, 0.67 g (0.03 equiv, 1.2 mmol) of Pd(dba)₂, 3.71 g (1.0 equiv, 38.6 mmol) of NaOtBu, 193 mL of toluene, 10.48 g (1.1 equiv, 42.4 mmol) of 4-bromodibenzofuran and 0.78 g (0.1 equiv, 3.9 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-18 (13.29 g, yield 81%).

FAB-MS measurement was conducted, and Intermediate IM-18 was identified by observing a mass number of m/z=425 as a molecular ion peak.

(3) Synthesis of Intermediate IM-19

Under an Ar atmosphere, to a 1,000 mL, three-neck flask, 20.00 g (76.0 mmol) of 1-bromodibenzothiophene, 13.07 g (1.1 equiv, 83.6 mmol) of 4-chlorophenylboronic acid, 31.51 g (3.0 equiv, 228.0 mmol) of K₂CO₃, 4.39 g (0.05 equiv, 3.8 mmol) of Pd(PPh₃)₄, and 532 mL of a mixture solution of toluene/EtOH/H₂O (4/2/1) were added in order and heated and stirred at about 80° C. After cooling to room temperature, the reaction solution was extracted with toluene. An aqueous layer was removed, and an organic layer was washed with a saturated saline solution and dried with MgSO₄. MgSO₄ was filtered and separated, and an organic layer was concentrated. The crude product thus obtained was separated by silica gel column chromatography (using a mixture solvent of hexane and toluene as a developing layer) to obtain Intermediate IM-19 (16.80 g, yield 75%).

FAB-MS measurement was conducted, and Intermediate IM-19 was identified by observing a mass number of m/z=294 as a molecular ion peak.

(4) Synthesis of Compound F9

Under an Ar atmosphere, to a 300 ml, three-neck flask, 10.00 g (23.5 mmol) of IM-18, 0.41 g (0.03 equiv, 0.7 mmol) of Pd(dba)₂, 4.52 g (2.0 equiv, 47.0 mmol) of NaOtBu, 117 mL of toluene, 7.62 g (1.1 equiv, 25.9 mmol) of IM-19 and 0.48 g (0.1 equiv, 2.4 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Compound F9 (11.73 g, yield 73%) as a solid.

FAB-MS measurement was conducted, and Compound F9 was identified by observing a mass number of m/z=683 as a molecular ion peak.

12. Synthesis of Compound F46

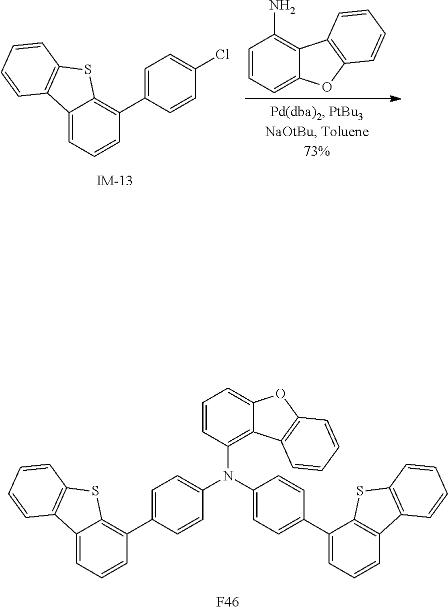

(1) Synthesis of Compound F46

Under an Ar atmosphere, to a 300 ml, three-neck flask, 4.00 g (21.8 mmol) of 1-aminodibenzofuran, 0.75 g (0.06 equiv, 1.3 mmol) of Pd(dba)₂, 8.39 g (4.0 equiv, 87.3 mmol) of NaOtBu, 109 mL of toluene, 14.16 g (2.2 equiv, 48.0 mmol) of IM-13 and 0.88 g (0.2 equiv, 4.4 mmol) of tBu₃P were added in order, and heated, refluxed, and stirred. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered and separated, and an organic layer was concentrated. The crude product thus obtained was separated by silica gel column chromatography (using a mixture solvent of hexane and toluene as a developing layer) to obtain Compound F46 (11.15 g, yield 73%) as a solid.

FAB-MS measurement was conducted, and Compound F46 was identified by observing a mass number of m/z=699 as a molecular ion peak.

(Example of Manufacturing Device)

Organic electroluminescence devices were manufactured using the Example Compounds and Comparative Compounds below as materials for a hole transport region.

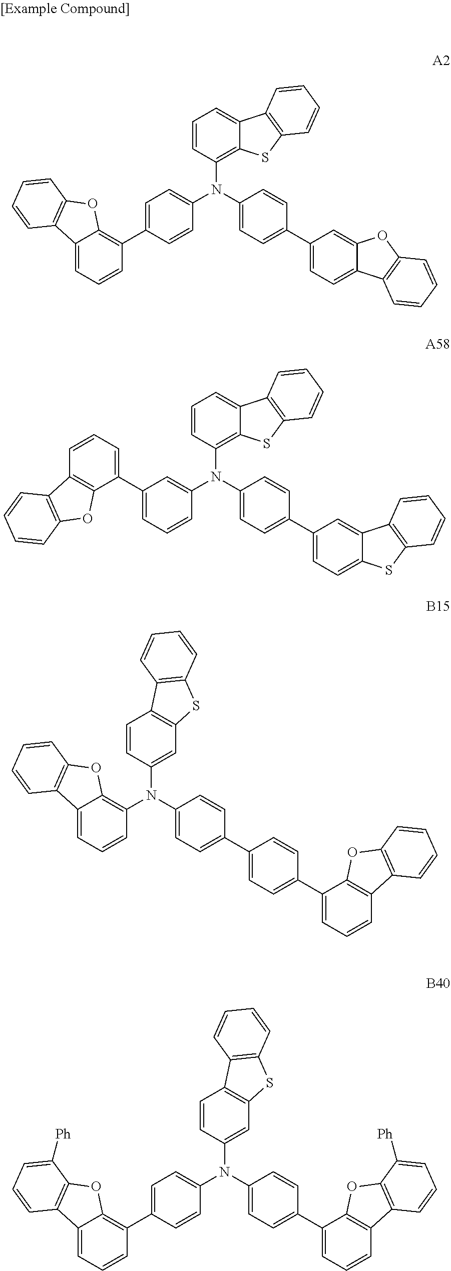 [SMILES: c1ccc2c(c1)oc1cc(-c3ccc(N(c4ccc(-c5cccc6c5oc5ccccc56)cc4)c4cccc5c4sc4ccccc45)cc3)ccc12 ; c1cc(-c2cccc3c2oc2ccccc23)cc(N(c2ccc(-c3ccc4sc5ccccc5c4c3)cc2)c2cccc3c2sc2ccccc23)c1 ; c1ccc2c(c1)oc1c(-c3ccc(-c4ccc(N(c5ccc6c(c5)sc5ccccc56)c5cccc6c5oc5ccccc56)cc4)cc3)cccc12 ; c1ccc(-c2cccc3c2oc2c(-c4ccc(N(c5ccc(-c6cccc7c6oc6c(-c8ccccc8)cccc67)cc5)c5ccc6c(c5)sc5ccccc56)cc4)cccc23)cc1]

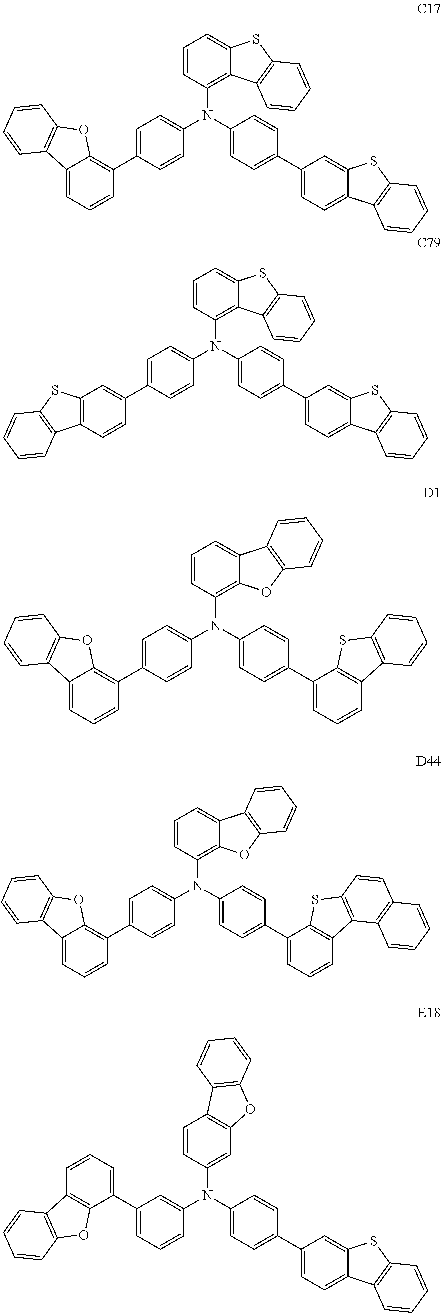 [SMILES: c1ccc2c(c1)oc1c(-c3ccc(N(c4ccc(-c5ccc6c(c5)sc5ccccc56)cc4)c4cccc5sc6ccccc6c45)cc3)cccc12 ; c1ccc2c(c1)sc1cc(-c3ccc(N(c4ccc(-c5ccc6c(c5)sc5ccccc56)cc4)c4cccc5sc6ccccc6c45)cc3)ccc12 ; c1ccc2c(c1)oc1c(-c3ccc(N(c4ccc(-c5cccc6c5sc5ccccc56)cc4)c4cccc5c4oc4ccccc45)cc3)cccc12 ; c1ccc2c(c1)ccc1sc3c(-c4ccc(N(c5ccc(-c6cccc7c6oc6ccccc67)cc5)c5cccc6c5oc5ccccc56)cc4)cccc3c12 ; c1cc(-c2cccc3c2oc2ccccc23)cc(N(c2ccc(-c3ccc4c(c3)sc3ccccc34)cc2)c2ccc3c(c2)oc2ccccc23)c1]

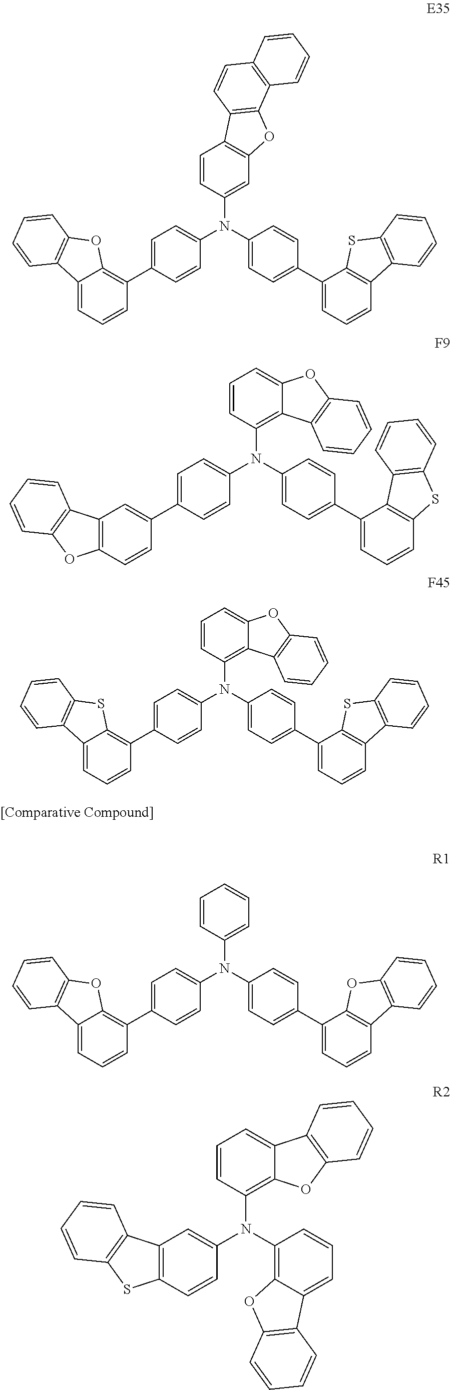

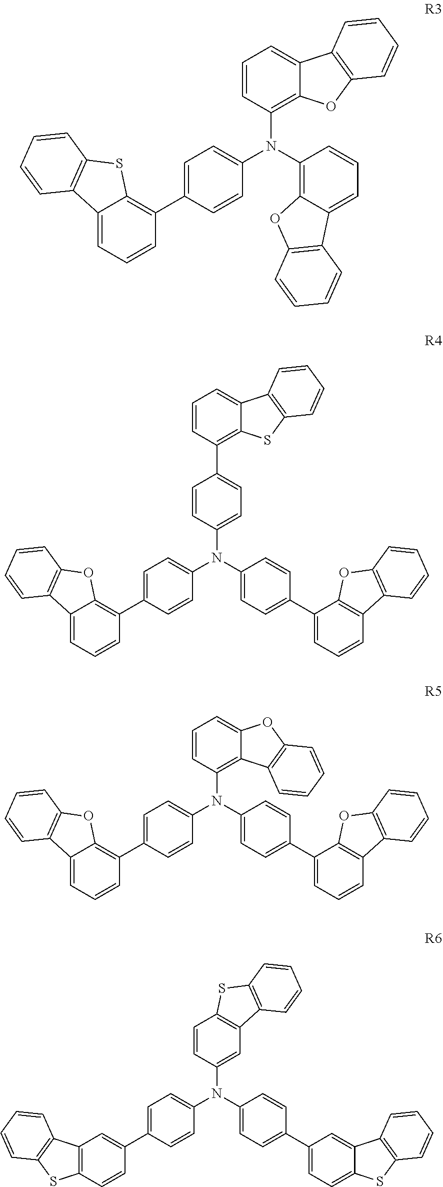 [SMILES: c1ccc2c(c1)oc1c(N(c3ccc(-c4cccc5c4sc4ccccc45)cc3)c3cccc4c3oc3ccccc34)cccc12 ; c1ccc2c(c1)oc1c(-c3ccc(N(c4ccc(-c5cccc6c5oc5ccccc56)cc4)c4ccc(-c5cccc6c5sc5ccccc56)cc4)cc3)cccc12 ; c1ccc2c(c1)oc1c(-c3ccc(N(c4ccc(-c5cccc6c5oc5ccccc56)cc4)c4cccc5oc6ccccc6c45)cc3)cccc12 ; c1ccc2c(c1)sc1ccc(-c3ccc(N(c4ccc(-c5ccc6sc7ccccc7c6c5)cc4)c4ccc5sc6ccccc6c5c4)cc3)cc12]

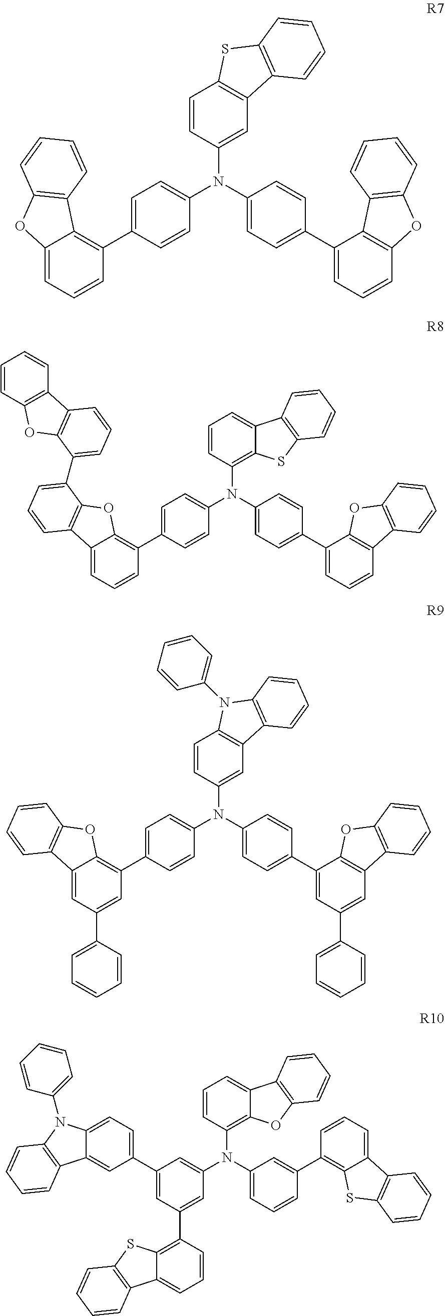 [SMILES: c1ccc2c(c1)oc1cccc(-c3ccc(N(c4ccc(-c5cccc6oc7ccccc7c56)cc4)c4ccc5sc6ccccc6c5c4)cc3)c12 ; c1ccc2c(c1)oc1c(-c3ccc(N(c4ccc(-c5cccc6c5oc5c(-c7cccc8c7oc7ccccc78)cccc56)cc4)c4cccc5c4sc4ccccc45)cc3)cccc12 ; c1ccc(-c2cc(-c3ccc(N(c4ccc(-c5cc(-c6ccccc6)cc6c5oc5ccccc56)cc4)c4ccc5c(c4)c4ccccc4n5-c4ccccc4)cc3)c3oc4ccccc4c3c2)cc1 ; c1ccc(-n2c3ccccc3c3cc(-c4cc(-c5cccc6c5sc5ccccc56)cc(N(c5cccc(-c6cccc7c6sc6ccccc67)c5)c5cccc6c5oc5ccccc56)c4)ccc32)cc1]

The organic electroluminescence devices of the Examples and Comparative Examples were manufactured by a method below. ITO with a thickness of about 150 nm was patterned on a glass substrate, and cleaned with ultrapure water, treated with UV ozone for about 10 minutes to form a first electrode. After that, 2-TNATA was deposited to a thickness of about 60 nm, and using the Example Compound or Comparative Compound, a hole transport layer with thickness of about 30 nm was formed. An emission layer with a thickness of about 25 nm was formed using ADN doped with 3% TBP, and on the emission layer, a layer with a thickness of about 25 nm was formed using Alq₃, and a layer with a thickness of about 1 nm was formed using LiF to form an electron transport region. A second electrode EL2 with a thickness of about 100 nm was formed using aluminum (Al). All layers were formed by a vacuum deposition method.

The measurement values according to Examples 1 to 12 and Comparative Examples 1 to 10 are shown in Table 1 below. The emission efficiency corresponds to values measured at 10 mA/cm², and half-life corresponds to test results at 1.0 mA/cm².

TABLE 1

| | | | Emission | |
|---|---|---|---|---|
| | | Voltage | efficiency | Life |
| | Hole transport layer | (V) | (%) | LT50(h) |
| Example 1 | Example | 5.6 | 7.6 | 1950 |
| | Compound A2 | | | |
| Example 2 | Example | 5.7 | 7.7 | 1900 |
| | Compound A58 | | | |
| Example 3 | Example | 5.5 | 7.4 | 2050 |
| | Compound B15 | | | |
| Example 4 | Example | 5.6 | 7.5 | 2100 |
| | Compound B40 | | | |
| Example 5 | Example | 5.6 | 7.9 | 1900 |
| | Compound C47 | | | |
| Example 6 | Example | 5.7 | 7.8 | 1950 |
| | Compound C70 | | | |
| Example 7 | Example | 5.4 | 7.7 | 1950 |
| | Compound D1 | | | |
| Example 8 | Example | 5.5 | 7.7 | 2000 |
| | Compound D44 | | | |
| Example 9 | Example | 5.6 | 7.6 | 2050 |
| | Compound E18 | | | |
| Example 10 | Example | 5.6 | 7.5 | 2150 |
| | Compound E35 | | | |
| Example 11 | Example | 5.7 | 8.0 | 1950 |
| | Compound F9 | | | |
| Example 12 | Example | 5.5 | 7.9 | 1900 |
| | Compound F46 | | | |
| Comparative | Comparative | 6.1 | 6.2 | 1550 |
| Example 1 | Compound R1 | | | |
| Comparative | Comparative | 6.0 | 6.1 | 1650 |
| Example 2 | Compound R2 | | | |
| Comparative | Comparative | 5.9 | 6.7 | 1700 |
| Example 3 | Compound R3 | | | |
| Comparative | Comparative | 6.1 | 6.5 | 1600 |
| Example 4 | Compound R4 | | | |
| Comparative | Comparative | 6.0 | 6.6 | 1650 |
| Example 5 | Compound R5 | | | |
| Comparative | Comparative | 6.2 | 6.2 | 1550 |
| Example 6 | Compound R6 | | | |
| Comparative | Comparative | 6.2 | 6.1 | 1550 |
| Example 7 | Compound R7 | | | |
| Comparative | Comparative | 6.4 | 6.2 | 1500 |
| Example 8 | Compound R8 | | | |
| Comparative | Comparative | 5.8 | 5.9 | 1600 |
| Example 9 | Compound R9 | | | |
| Comparative | Comparative | 5.9 | 6.1 | 1550 |
| Example 10 | Compound R10 | | | |

Referring to Table 1 above, it could be confirmed that Examples 1 to 12 achieved a lower voltage, longer life and higher efficiency at the same time when compared with Comparative Examples 1 to 10.

The amine compound according to an embodiment is used in a hole transport region to contribute to the decrease of a driving voltage, the increase of efficiency and life of an organic electroluminescence device. In the amine compound according to an embodiment, three dibenzoheterole groups are bonded to nitrogen via two linkers and one direct linkage. Accordingly, the amine compound according to an embodiment has excellent balance between the glass transition temperature and deposition temperature and improved heat resistance and charge tolerance. A heteroatom included in the dibenzoheterole skeleton improves the hole transport capacity of a whole molecule, and the recombination probability of holes and electrons in an emission layer is increased, and high emission efficiency may be achieved.

Comparative Example 1 corresponded to an amine material having two dibenzofuran groups, but when compared with the materials shown in Examples 1 to 12, the dibenzoheterole groups were small, and hole transport capacity was insufficient, and due to the delay of the injection of holes into an emission layer, emission efficiency was deteriorated in contrast to the Examples.

All of Comparative Examples 2 to 4 corresponded to amine materials having three dibenzoheterole groups, but both device efficiency and life were deteriorated when compared with the Examples.

When compared with the materials shown in Examples 1 to 12, it is thought that the connecting groups between dibenzoheterole and a nitrogen atom were small in Comparative Examples 2 and 3, and the glass transition temperature of the materials themselves was insufficient, the materials were deteriorated during continuous driving, the conjugation length of a HOMO orbital was short, and the stability in a radical state was insufficient.

Comparative Example 4 was an amine material in which all three dibenzoheterole groups were bonded to a nitrogen atom via connecting groups, but it is thought that intermolecular stacking was increased, the elevation of the deposition temperature of the material and the deterioration of layer-forming properties were induced, and accordingly, the material was deteriorated.

Comparative Example 5 was an amine material in which all heterocyclic groups at terminals were dibenzofuran groups, but when compared with the Examples, both device efficiency and life were degraded. An oxygen atom included in the dibenzofuran had high electronegativity, and if three dibenzofuran groups are introduced in the same molecule, the electron density of a nitrogen atom may be excessively degraded, and stability during applying electric current and driving may be deteriorated. As shown in the Examples, in case where at least one among three dibenzoheterole groups was a dibenzothiophene group, since a sulfur atom had smaller electronegativity than an oxygen atom, the destabilization due to the decrease of electron density of central nitrogen atom was solved, and excellent device properties could be shown.

Comparative Examples 6 and 7 were amine materials in which all dibenzoheterole groups directly bonded to a nitrogen atom were bonded at position 2, and when compared with the Examples, both device efficiency and life were degraded. If the dibenzoheterole makes a direct bond with the nitrogen atom at position 2, a heteroatom included in the dibenzoheterole and the nitrogen atom may be positioned at a para position, and the stability in a radical state may be degraded. As shown in Examples 2 and 11, in case where the dibenzoheterole was bonded to the nitrogen atom via a connecting group even at position 2, the number of interposed bond therebetween increased, and instability in a radical state was solved, and excellent device properties could be shown.

Comparative Example 8 corresponded to an amine material in which a dibenzoheterole group is additionally substituted at a dibenzoheterole group, and had a twisted stereostructure between two heterocycles. Accordingly, stability at high temperature conditions was low, and according to the increase of the deposition temperature, decomposition occurred during deposition. When compared with the Examples, both device efficiency and life were degraded.

Comparative Examples 9 and 10 corresponded to amine materials including a carbazole group, and according to the collapse of carrier balance, both device efficiency and life were degraded when compared with the Examples.

The amine compound according to an embodiment is used in a hole transport region and contributes to the decrease of a driving voltage and the increase of efficiency and life of an organic electroluminescence device.

The luminescence device according to an embodiment has excellent efficiency.

The amine compound according to an embodiment may be used as a material for a hole transport region of a luminescence device, and by using the same, the efficiency of the luminescence device may be improved.

Embodiments have been disclosed herein, and although terms are employed, they are used and are to be interpreted in a generic and descriptive sense only and not for purpose of limitation. In some instances, as would be apparent by one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the following claims.

What is claimed is:

1. A luminescence device, comprising:
a first electrode;
a hole transport region disposed on the first electrode;
an emission layer disposed on the hole transport region;
an electron transport region disposed on the emission layer; and
a second electrode disposed on the electron transport region,
wherein the hole transport region comprises an amine compound represented by Formula 1: 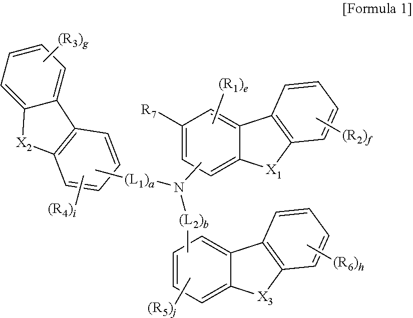
wherein in Formula 1,
X₁, X₂, and X₃ are each independently O or S,
R₁ to R₆ are each independently a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, or are combined with an adjacent group to form a ring,
R₇ is a hydrogen atom, a deuterium atom, a halogen atom, or a substituted or unsubstituted alkyl group of 1 to 20 carbon atoms,
L₁ and L₂ are each independently a substituted or unsubstituted arylene group of 6 to 30 ring-forming carbon atoms, where a heteroaryl group is excluded,
a and b are each independently an integer from 1 to 3,
e is an integer from 0 to 2,
f to h are each independently an integer from 0 to 4,
i and j are each independently an integer from 0 to 3, and
X₁, X₂, and X₃ are not O at the same time.

2. The luminescence device of claim 1, wherein
the hole transport region comprises:
a hole injection layer disposed on the first electrode; and
a hole transport layer disposed on the hole injection layer, and
the hole transport layer comprises the amine compound represented by Formula 1.

3. The luminescence device of claim 1, wherein
the hole transport region comprises:
a hole transport layer disposed on the first electrode; and
an electron blocking layer disposed on the hole transport layer, and
the electron blocking layer comprises the amine compound represented by Formula 1.

4. The luminescence device of claim 1, wherein Formula 1 is represented by Formula 2: 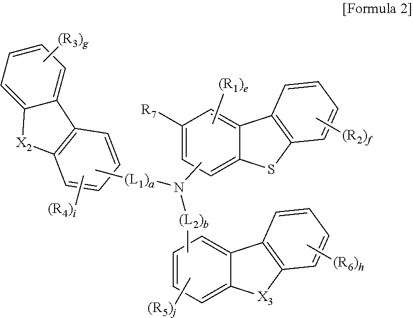
wherein in Formula 2,
X₂, X₃, R₁ to R₇, L₁, L₂, a, b, and e to j are the same as defined in connection with Formula 1.

5. The luminescence device of claim 1, wherein Formula 1 is represented by Formula 3: 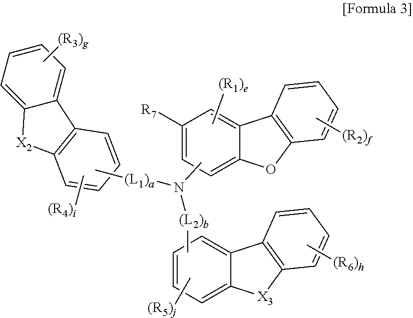
wherein in Formula 3,
X₂, X₃, R₁ to R₇, L₁, L₂, a, b, and e to j are the same as defined in connection with Formula 1.

6. The luminescence device of claim 4, wherein Formula 2 is represented by one of Formula 4-1 to Formula 4-3: 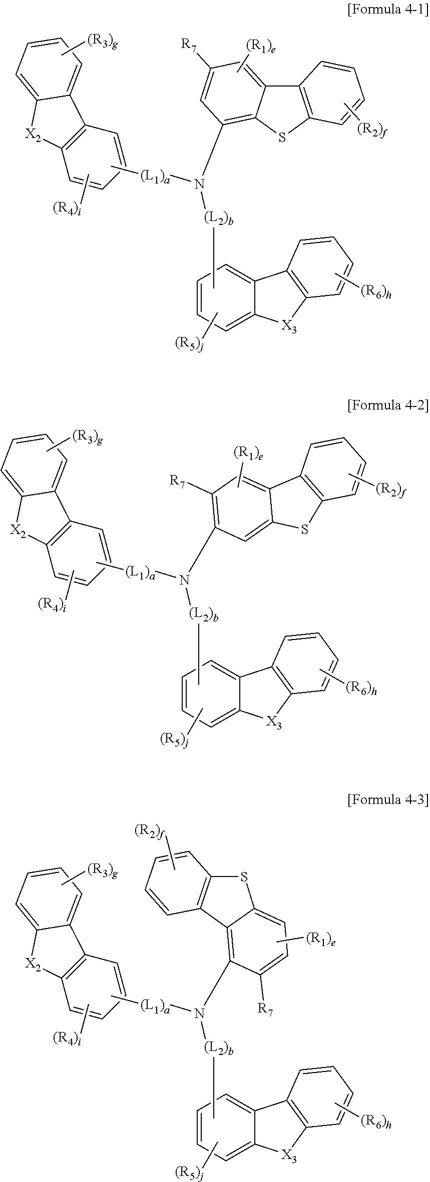
wherein in Formula 4-1 to Formula 4-3,
X₂, X₃, R₁ to R₇, L₁, L₂, a, b, and e to j are the same as defined in connection with Formula 2.

7. The luminescence device of claim 5, wherein Formula 3 is represented by one of Formula 5-1 to Formula 5-3: 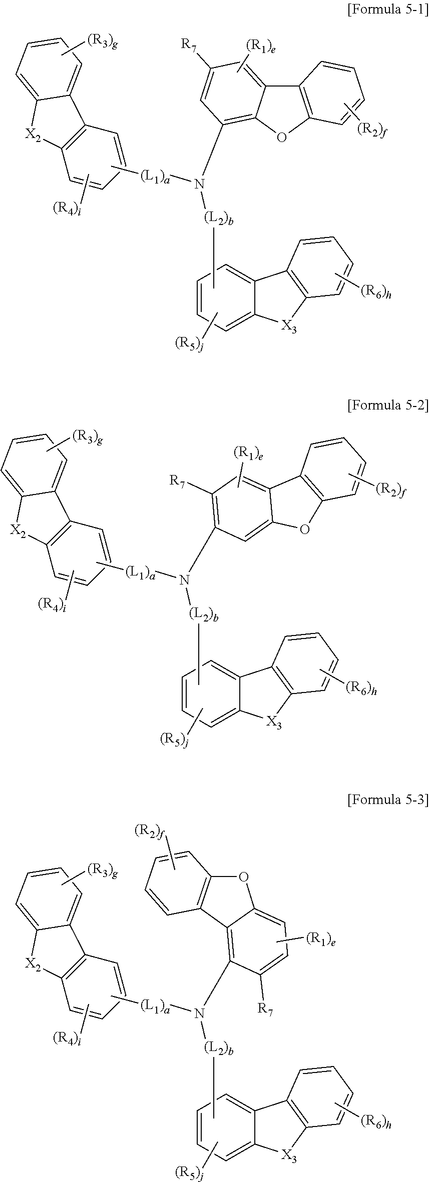
wherein in Formula 5-1 to Formula 5-3,
X₂, X₃, R₁ to R₇, L₁, L₂, a, b, and e to j are the same as defined in connection with Formula 3.

8. The luminescence device of claim 1, wherein
a and b are each 1, and
L₁ and L₂ are each independently a substituted or unsubstituted phenylene group, a substituted or unsubstituted biphenylene group, a substituted or unsubstituted terphenyl group, a substituted or unsubstituted naphthylene group, or a substituted or unsubstituted phenanthrylene group.

9. The luminescence device of claim 1, wherein L₁ and L₂ are each independently a group represented by one of L-1 to L-11: 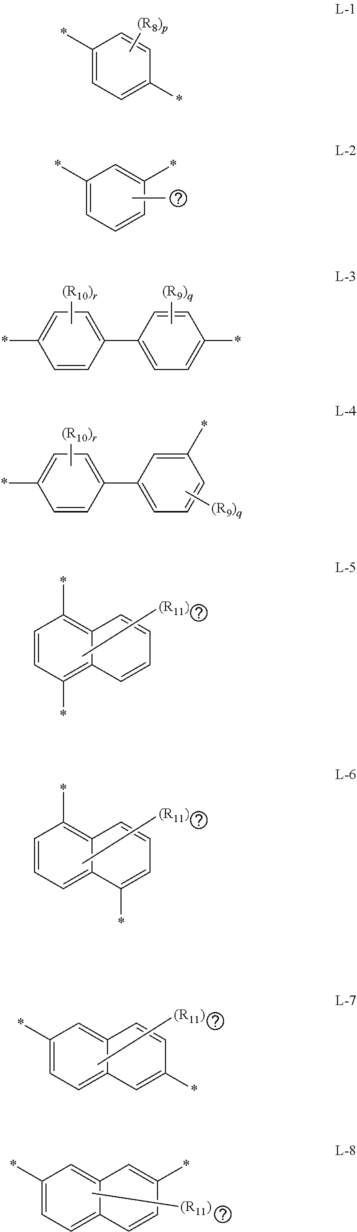 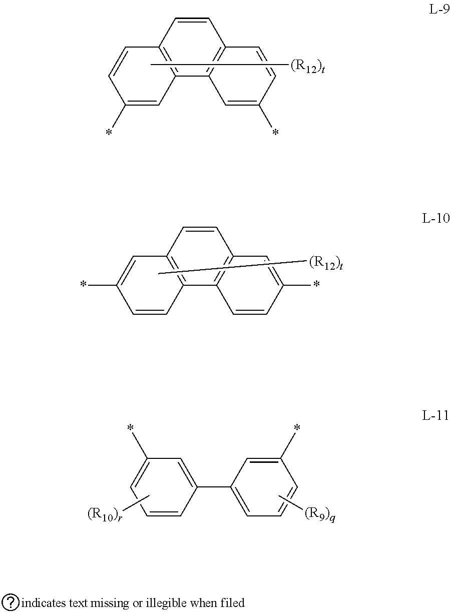
wherein in L-1 to L-11,
R₈ to R₁₂ are each independently a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms,
p to r are each independently an integer from 0 to 4,
s is an integer from 0 to 6,
t is an integer from 0 to 8, and
* indicates a binding site to a neighboring atom.

10. The luminescence device of claim 1, wherein the amine compound represented by Formula 1 is at least one selected from Compound Group 1:

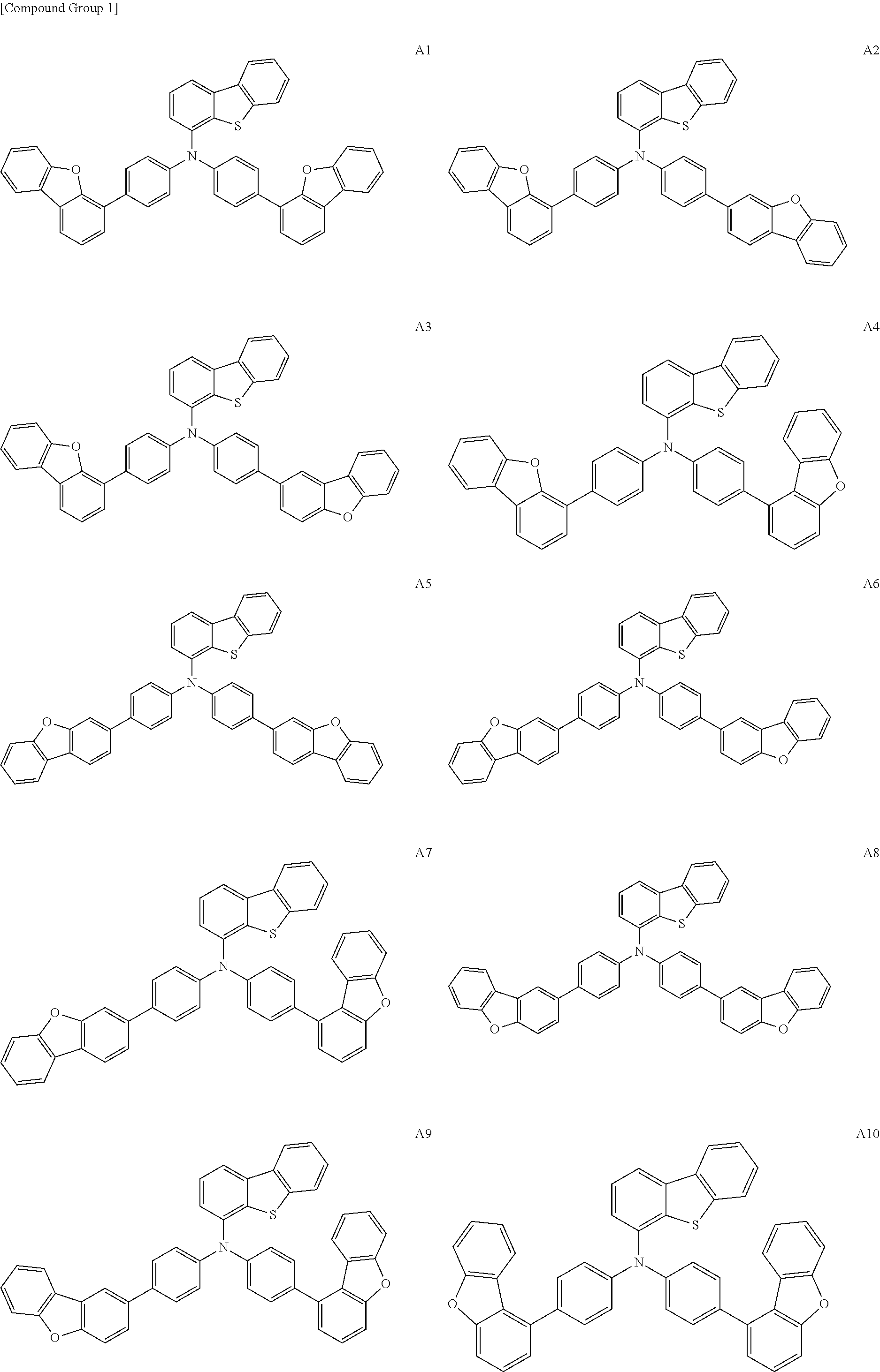

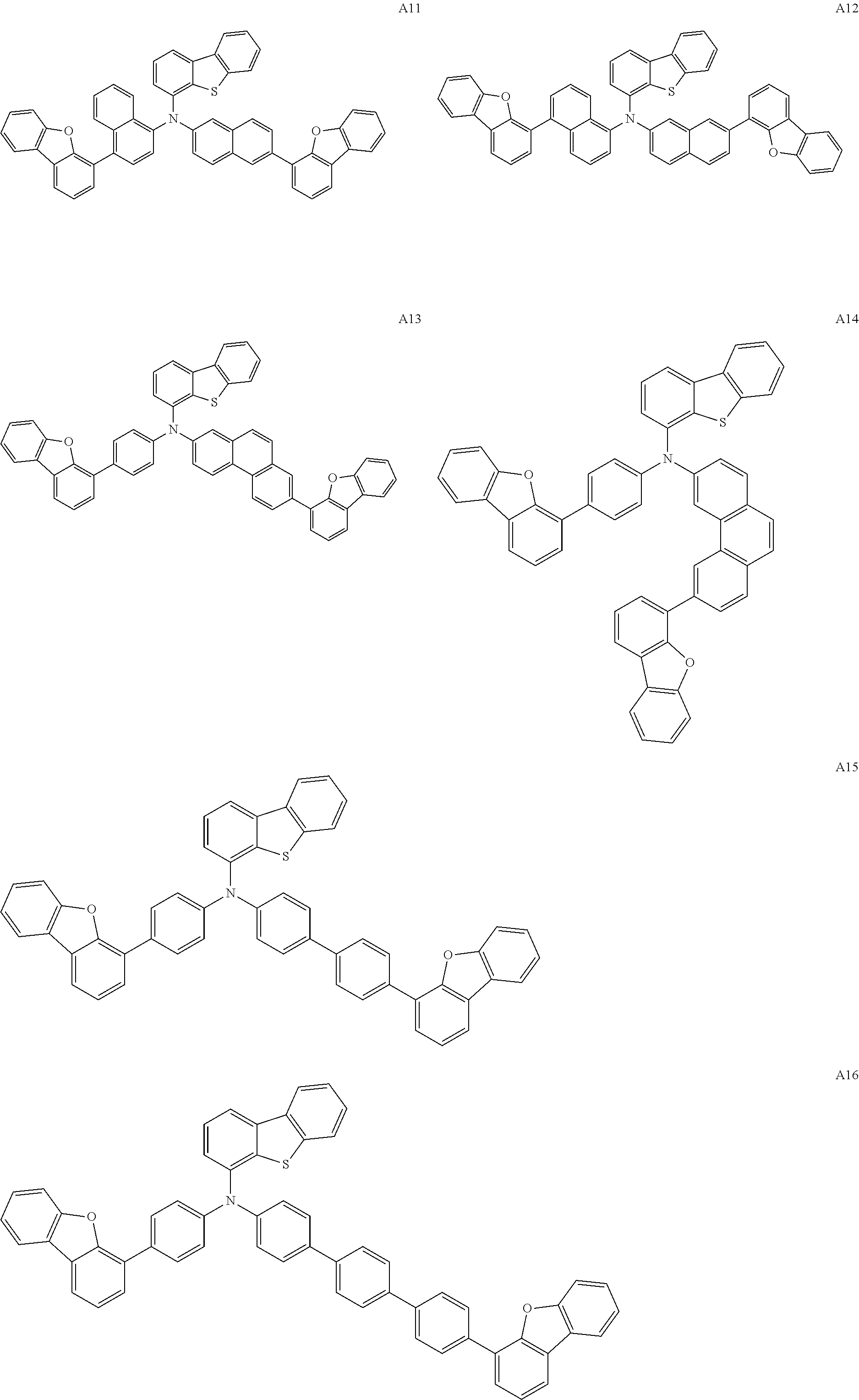

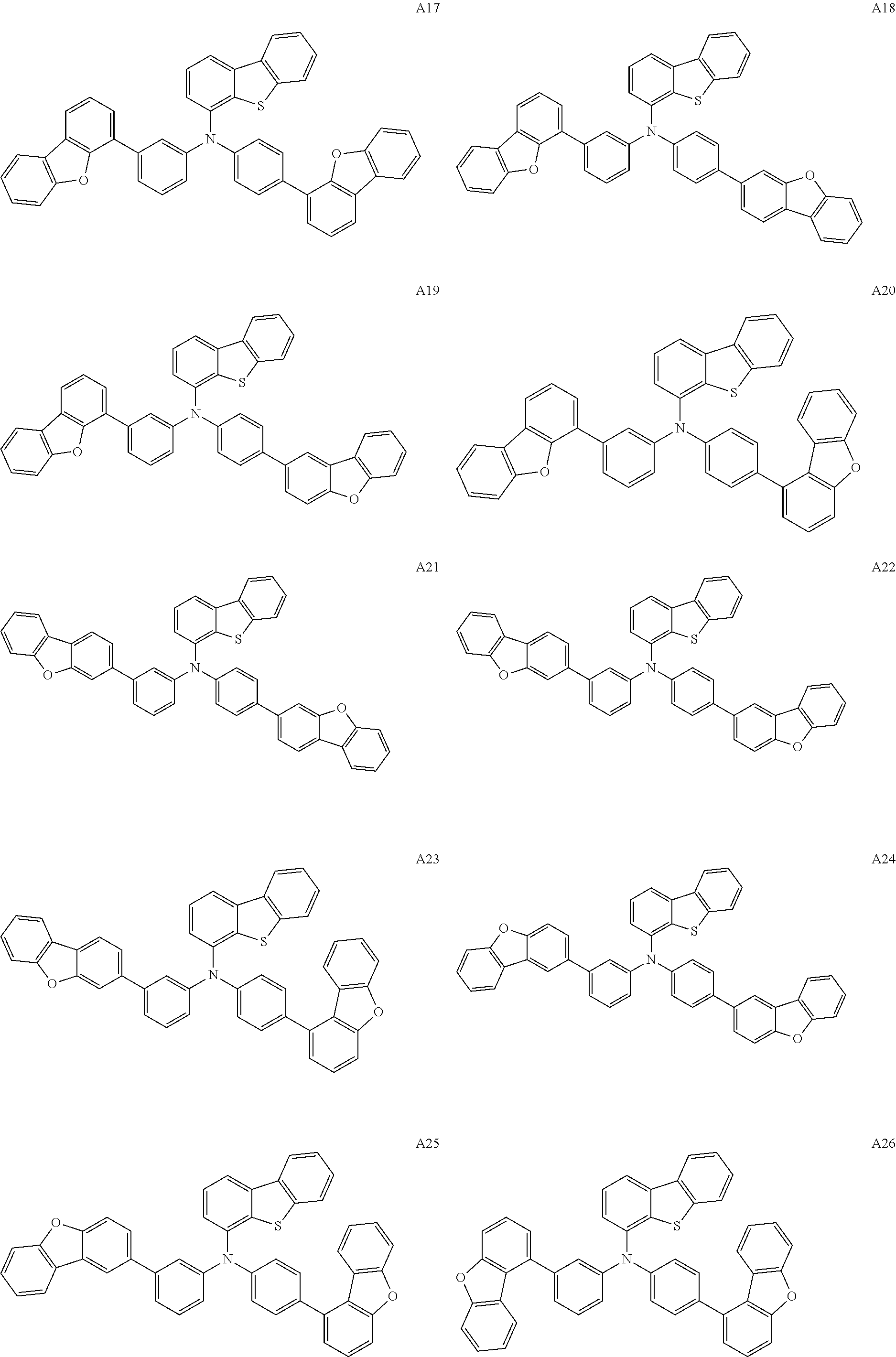

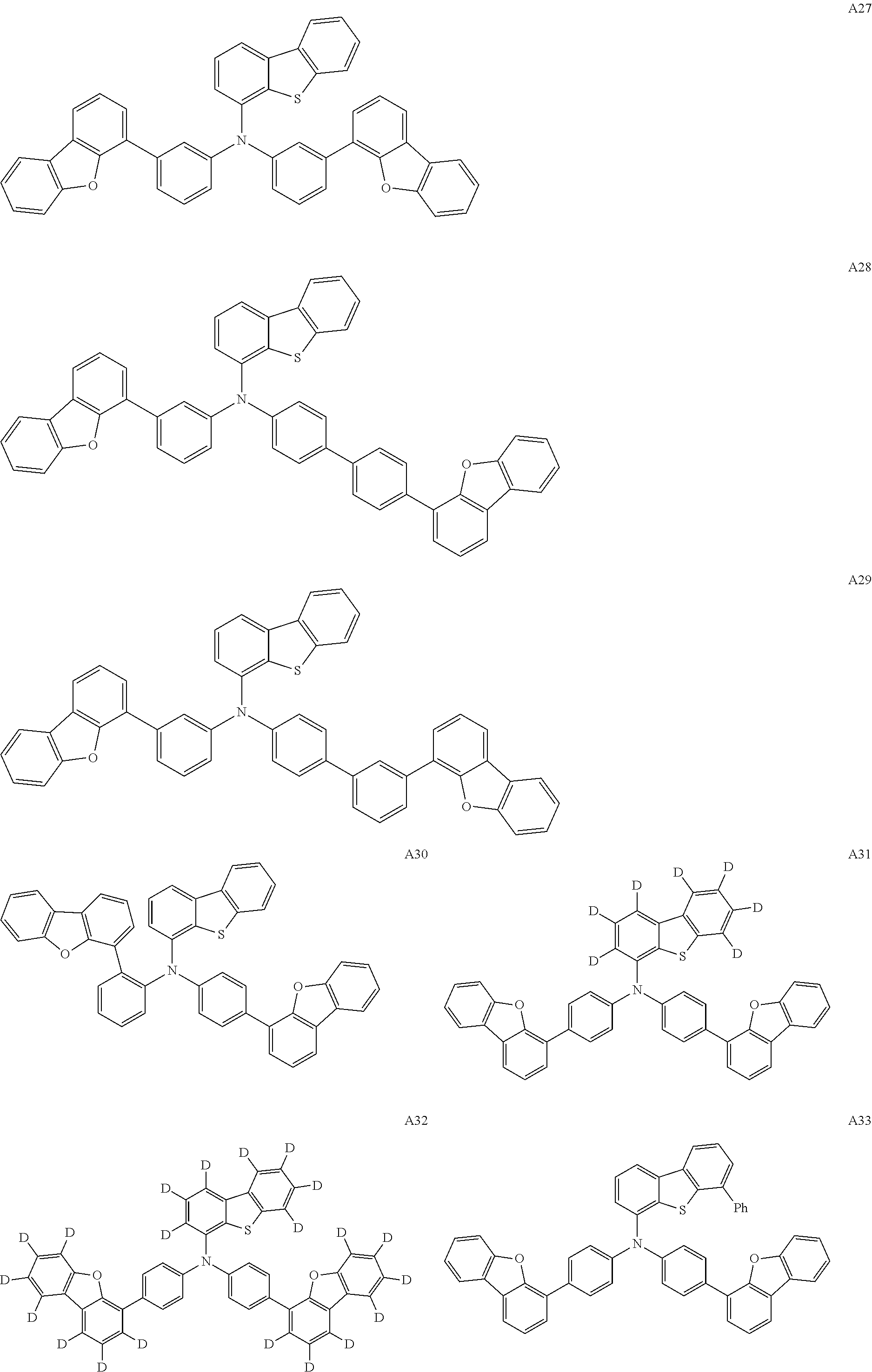

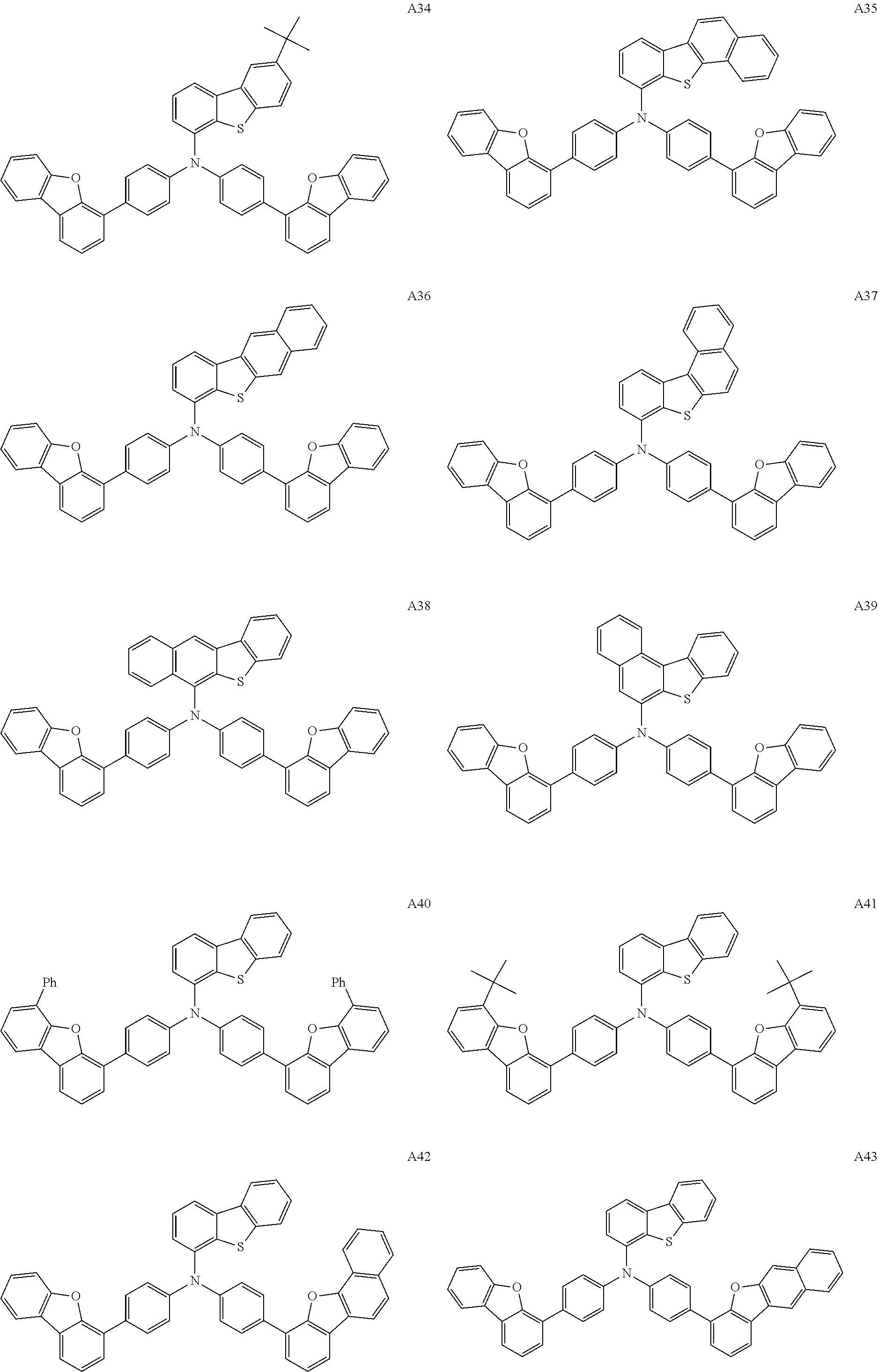

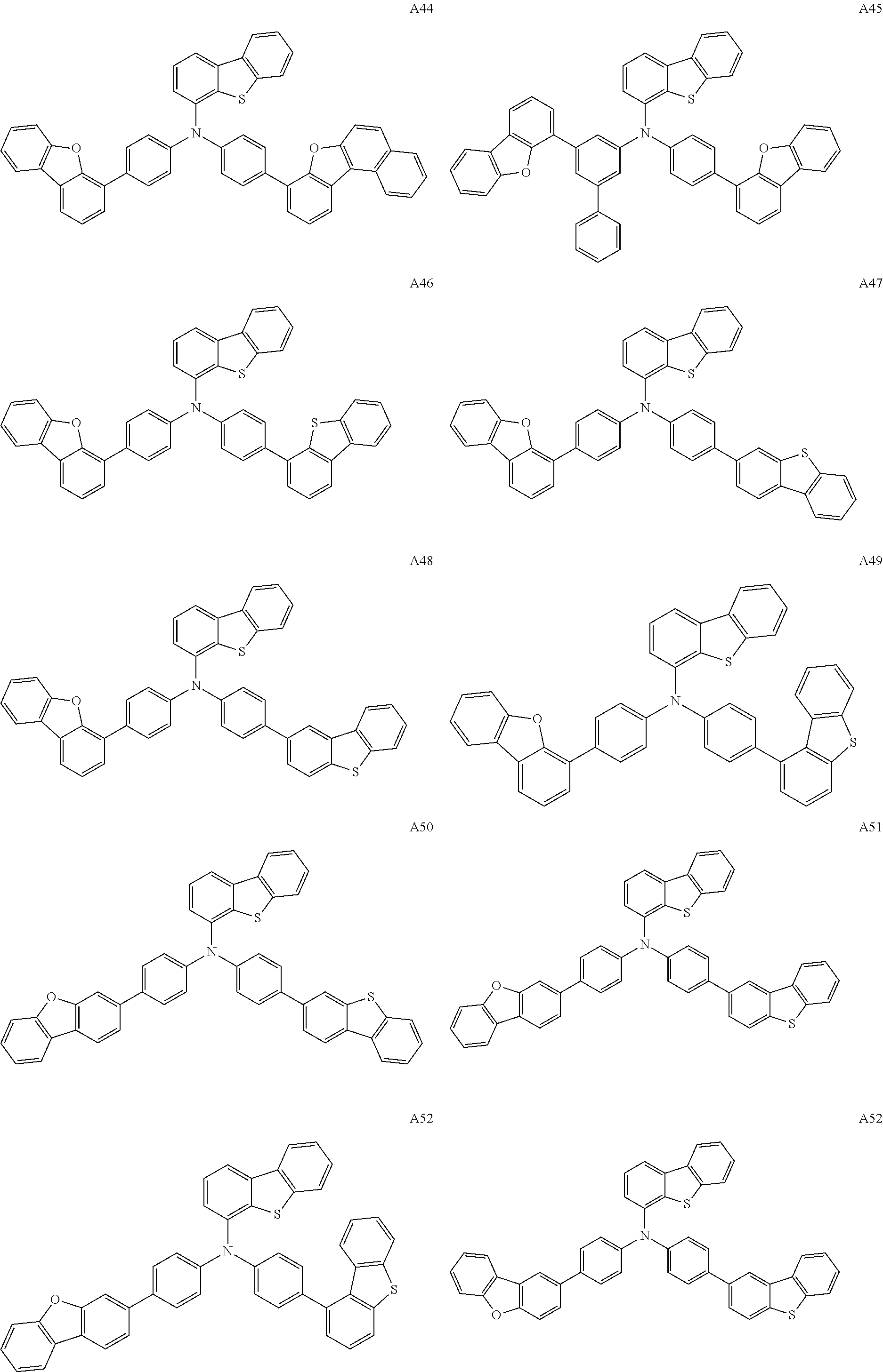

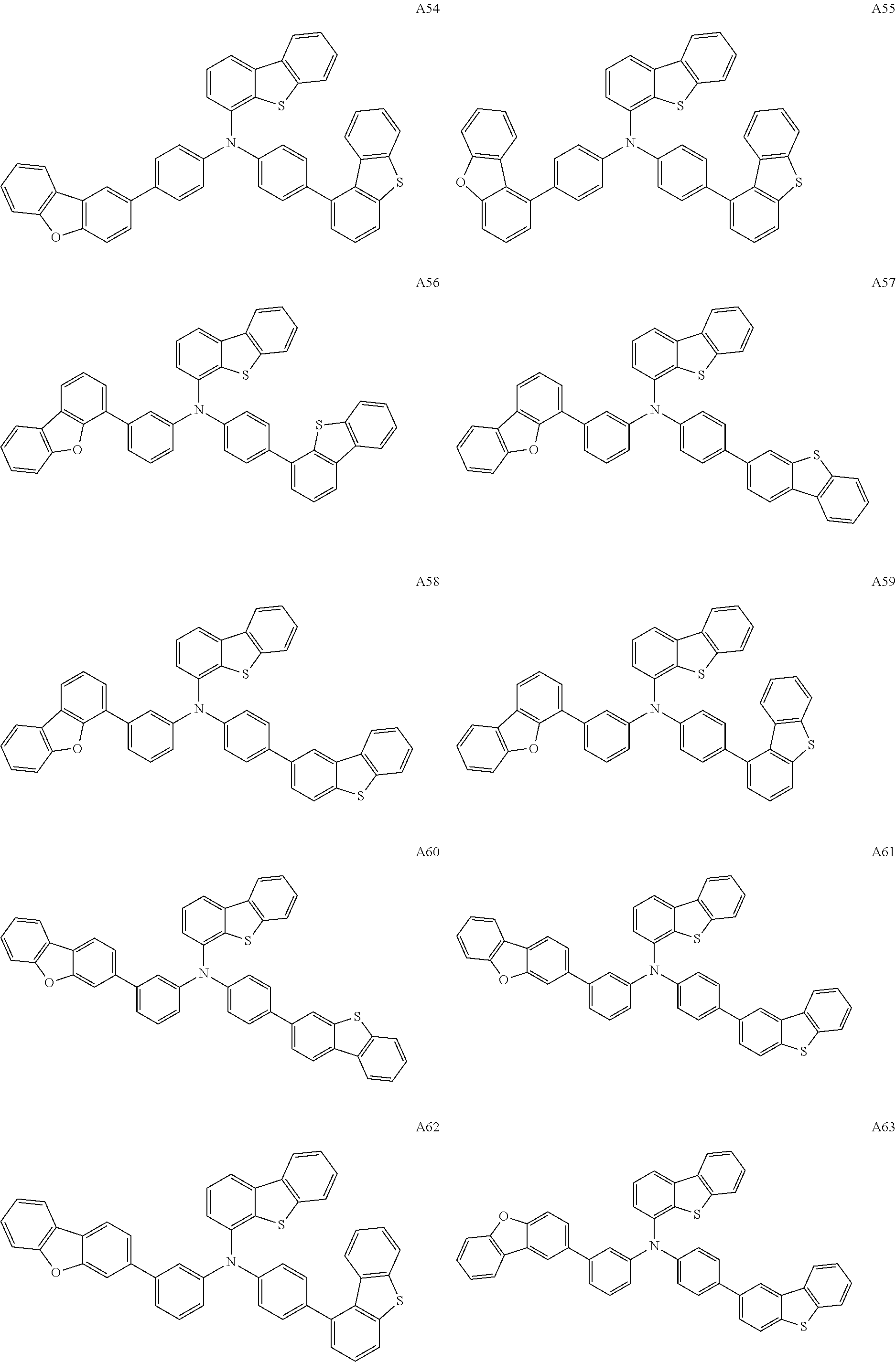

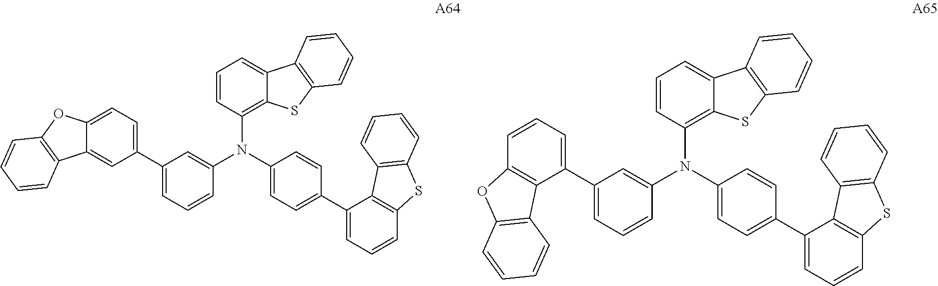 [SMILES: c1cc(-c2ccc3oc4ccccc4c3c2)cc(N(c2ccc(-c3cccc4sc5ccccc5c34)cc2)c2cccc3c2sc2ccccc23)c1 ; c1cc(-c2cccc3oc4ccccc4c23)cc(N(c2ccc(-c3cccc4sc5ccccc5c34)cc2)c2cccc3c2sc2ccccc23)c1]

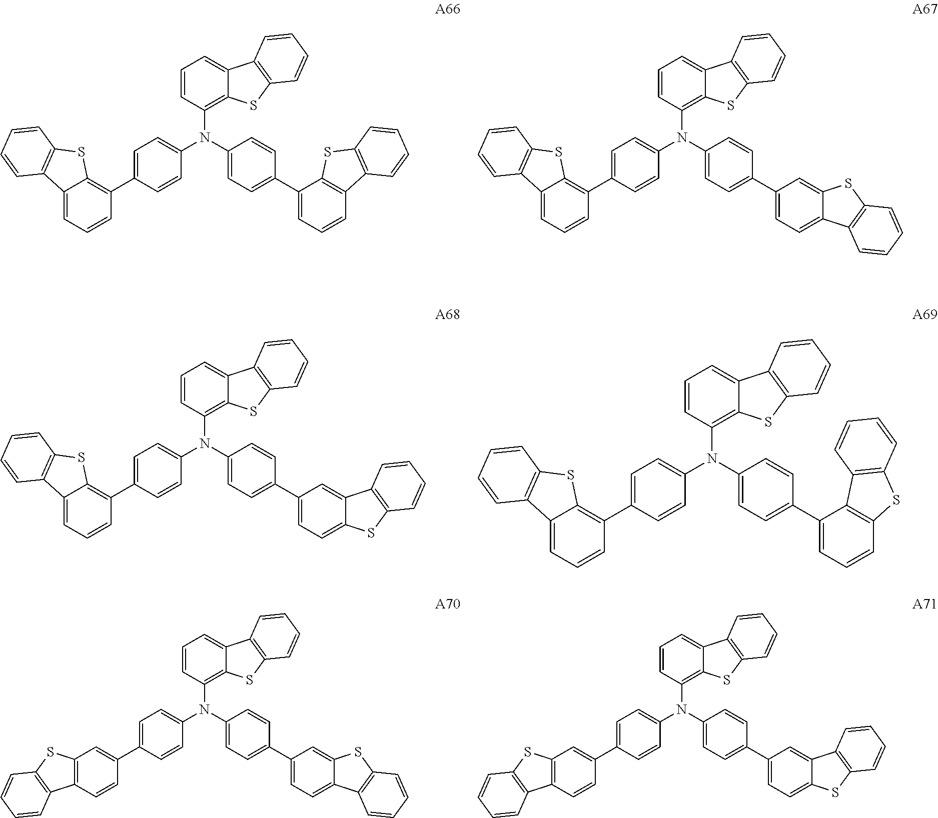

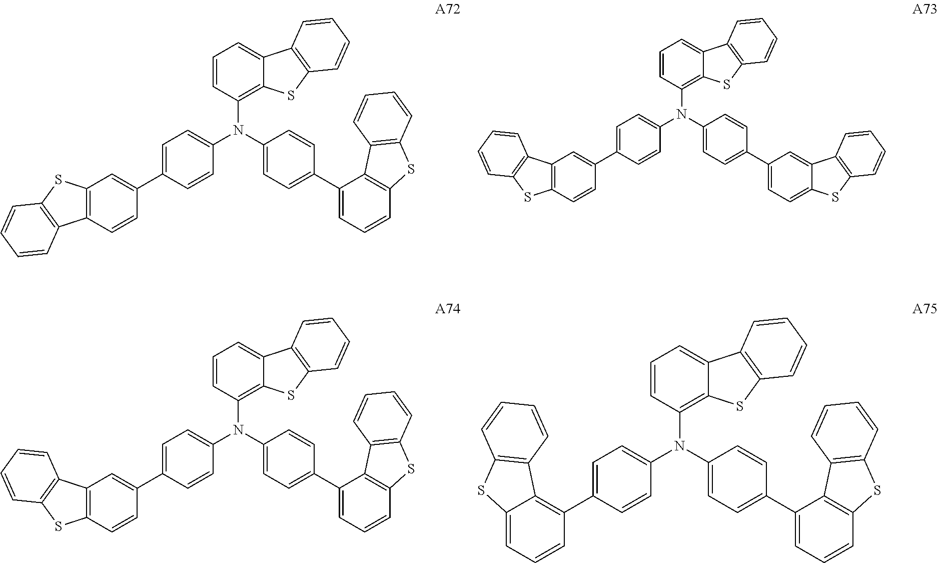 [SMILES: c1ccc2c(c1)sc1cc(-c3ccc(N(c4ccc(-c5cccc6sc7ccccc7c56)cc4)c4cccc5c4sc4ccccc45)cc3)ccc12 ; c1ccc2c(c1)sc1ccc(-c3ccc(N(c4ccc(-c5ccc6sc7ccccc7c6c5)cc4)c4cccc5c4sc4ccccc45)cc3)cc12 ; c1ccc2c(c1)sc1ccc(-c3ccc(N(c4ccc(-c5cccc6sc7ccccc7c56)cc4)c4cccc5c4sc4ccccc45)cc3)cc12 ; c1ccc2c(c1)sc1c(N(c3ccc(-c4cccc5sc6ccccc6c45)cc3)c3ccc(-c4cccc5sc6ccccc6c45)cc3)cccc12]

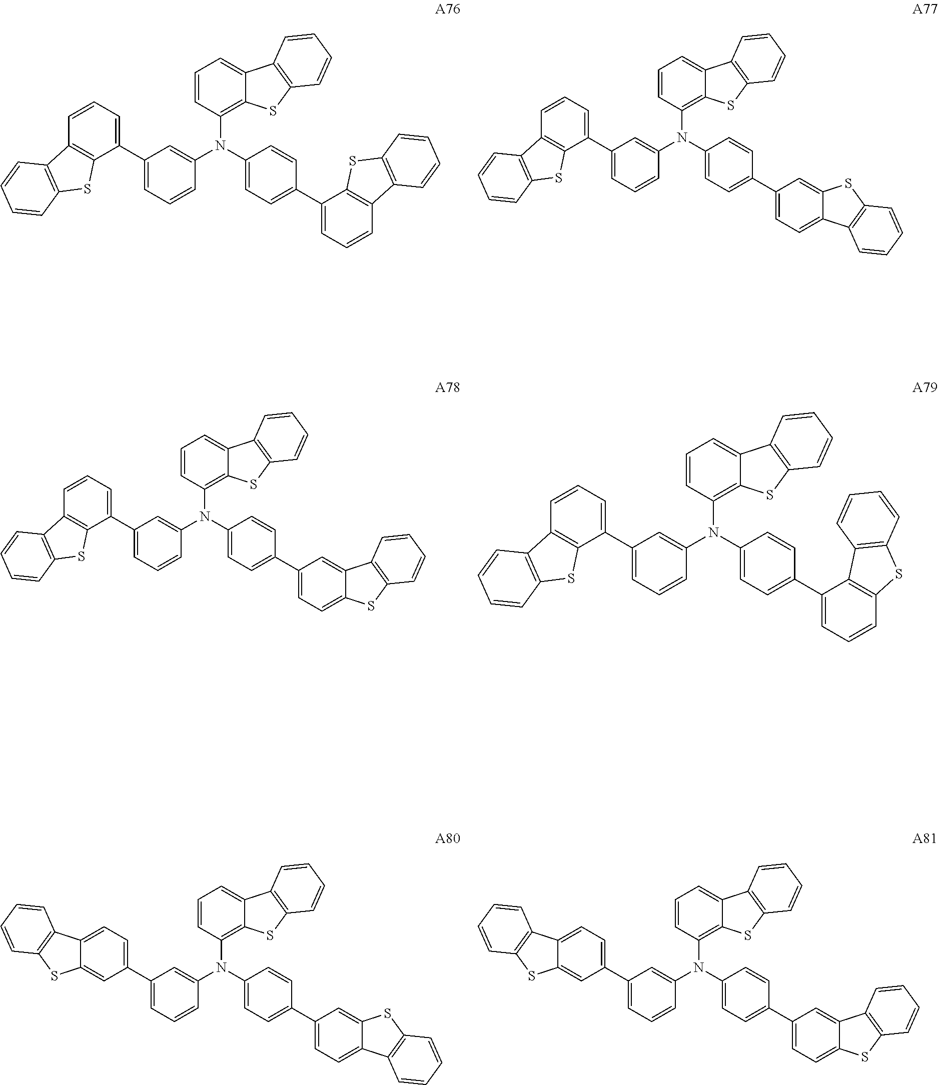

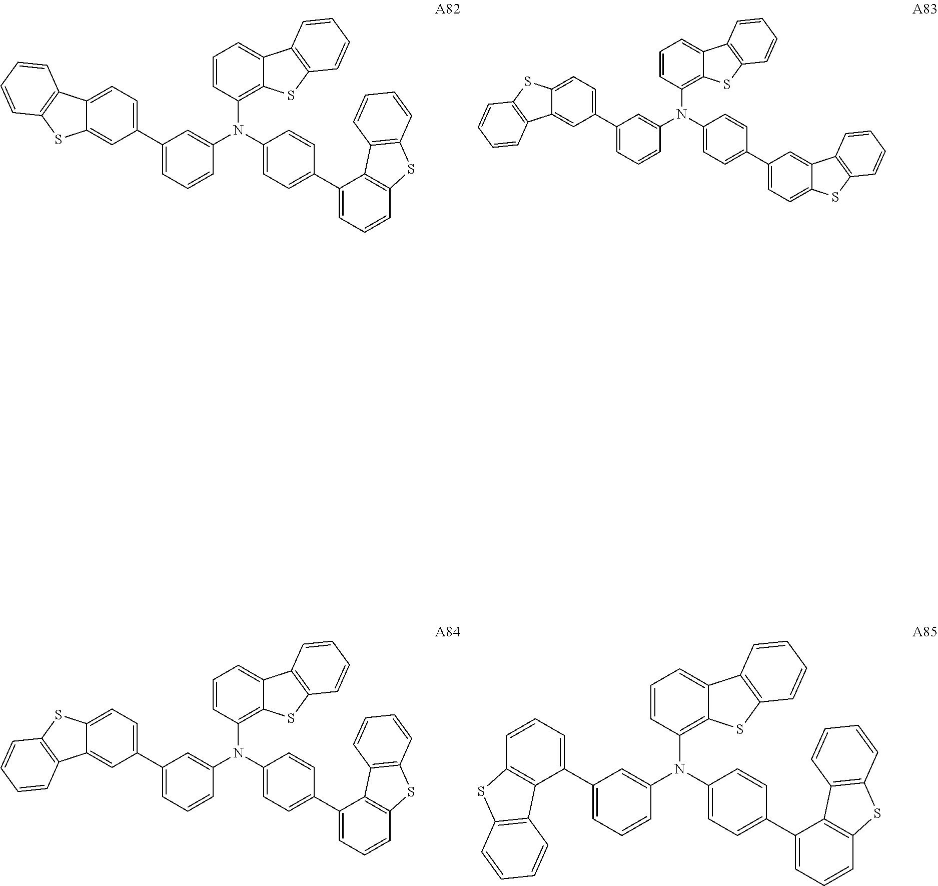 [SMILES: c1cc(-c2ccc3c(c2)sc2ccccc23)cc(N(c2ccc(-c3cccc4sc5ccccc5c34)cc2)c2cccc3c2sc2ccccc23)c1 ; c1cc(-c2ccc3sc4ccccc4c3c2)cc(N(c2ccc(-c3ccc4sc5ccccc5c4c3)cc2)c2cccc3c2sc2ccccc23)c1 ; c1cc(-c2ccc3sc4ccccc4c3c2)cc(N(c2ccc(-c3cccc4sc5ccccc5c34)cc2)c2cccc3c2sc2ccccc23)c1 ; c1cc(-c2cccc3sc4ccccc4c23)cc(N(c2ccc(-c3cccc4sc5ccccc5c34)cc2)c2cccc3c2sc2ccccc23)c1]

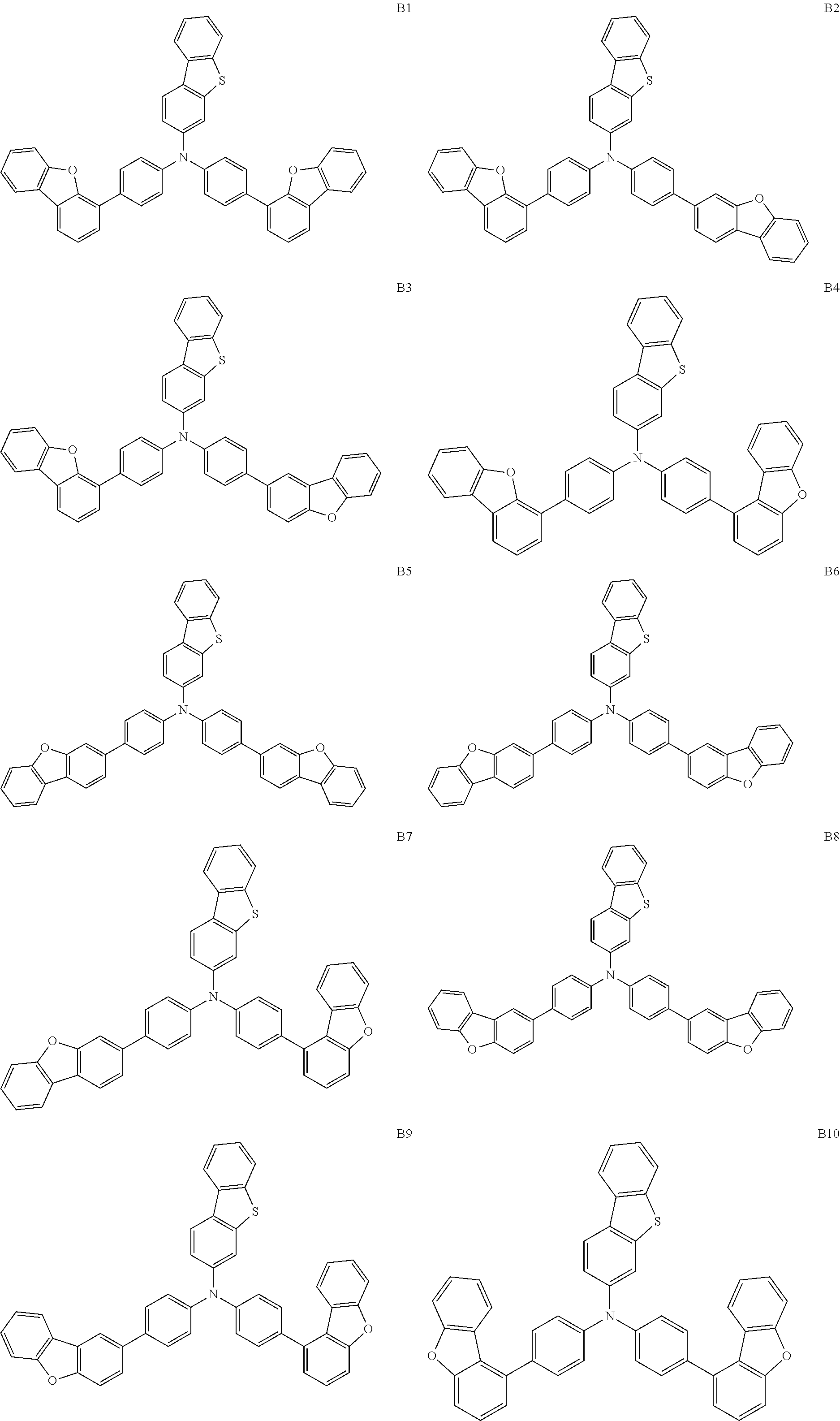

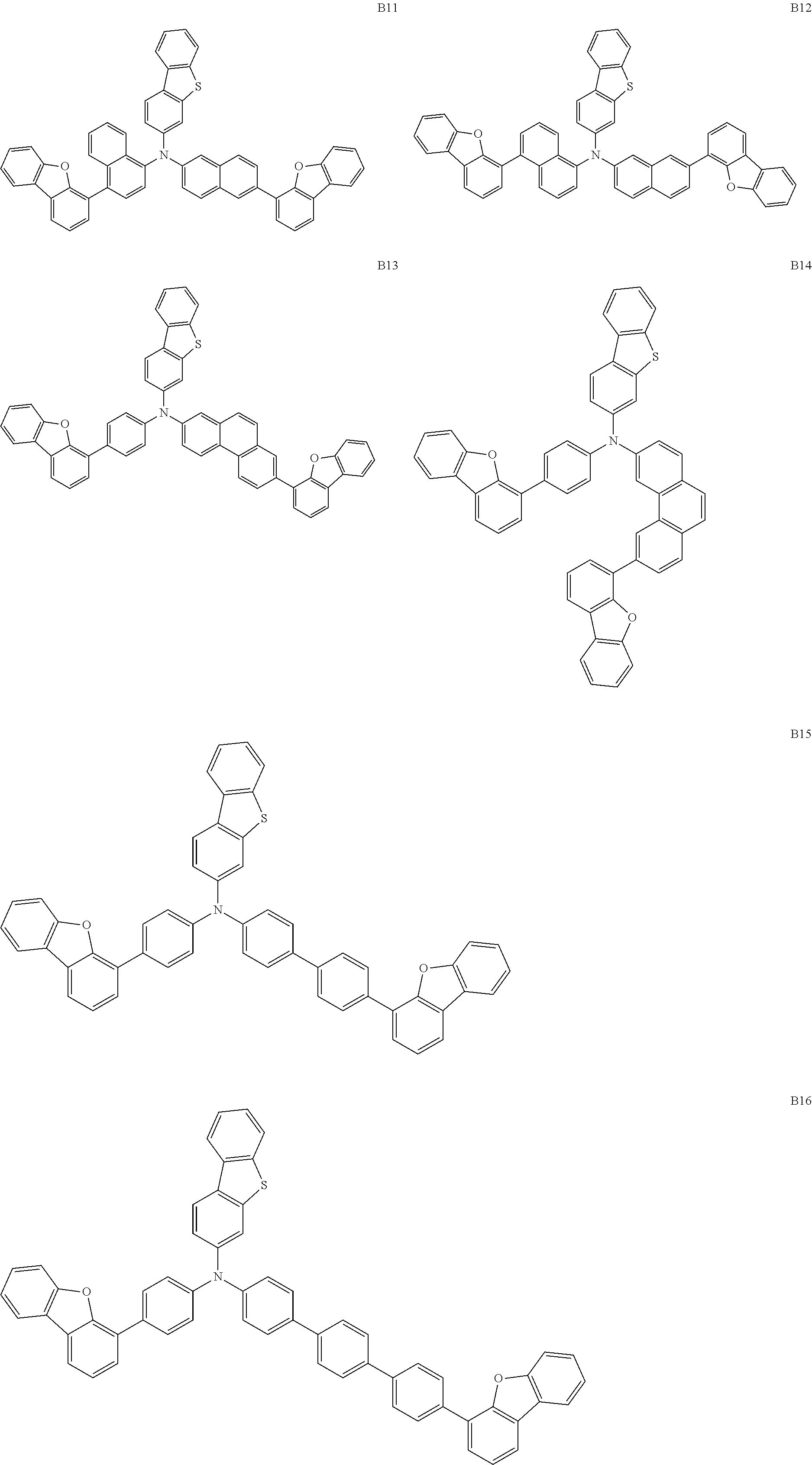

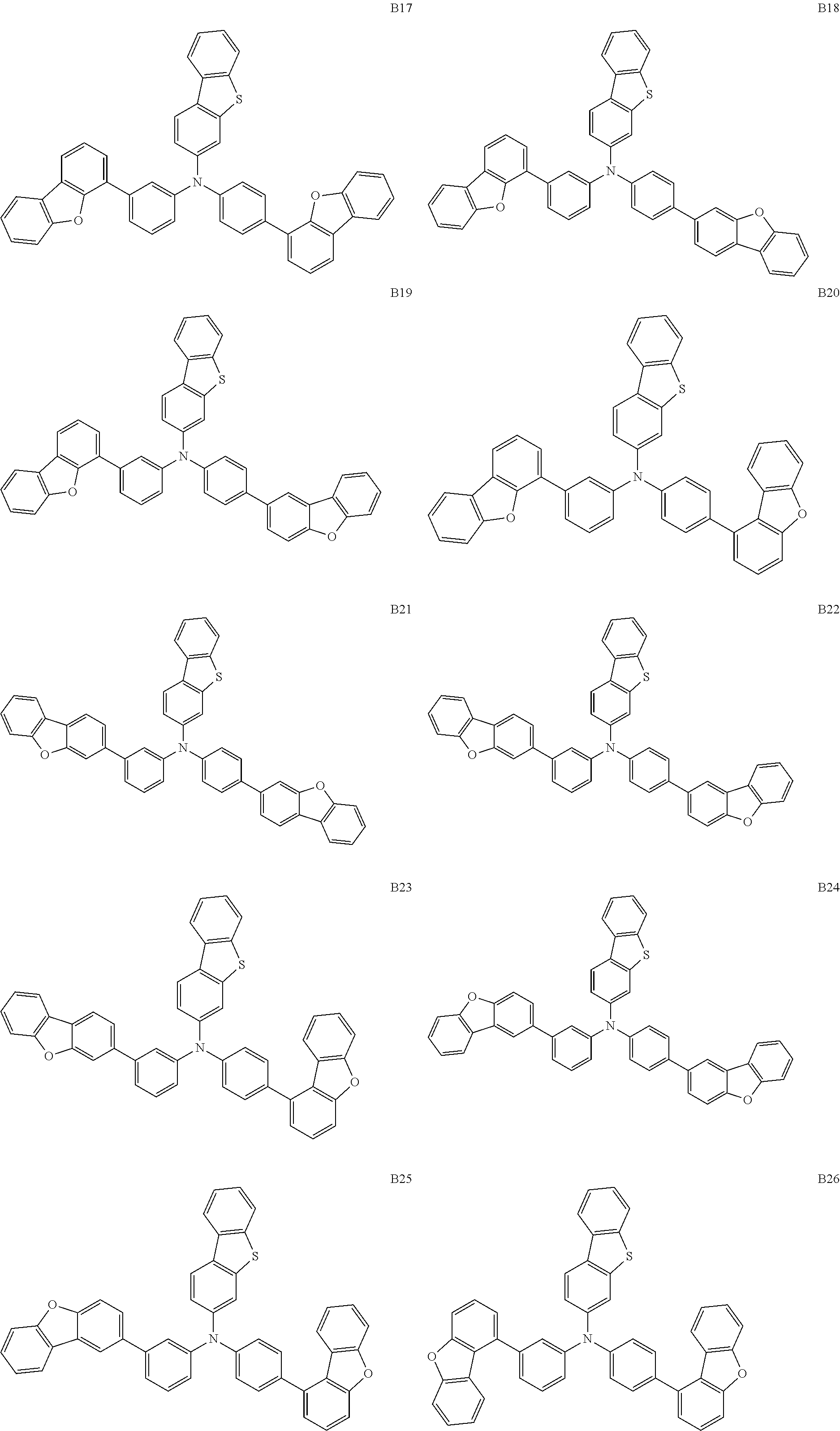

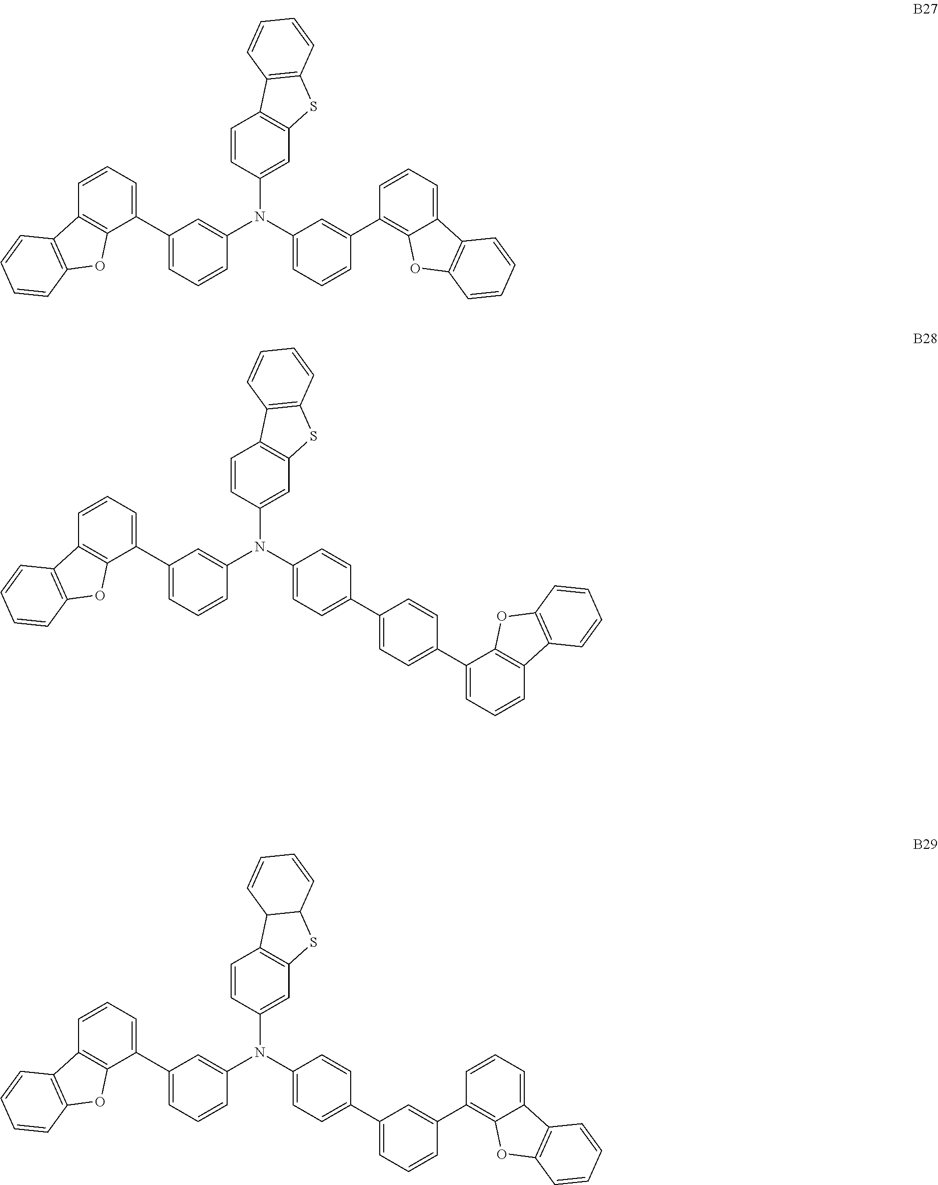 [SMILES: c1cc(-c2cccc3c2oc2ccccc23)cc(N(c2cccc(-c3cccc4c3oc3ccccc34)c2)c2ccc3c(c2)sc2ccccc23)c1 ; c1cc(-c2cccc3c2oc2ccccc23)cc(N(c2ccc(-c3ccc(-c4cccc5c4oc4ccccc45)cc3)cc2)c2ccc3c(c2)sc2ccccc23)c1 ; C1=CC2Sc3cc(N(c4ccc(-c5cccc(-c6cccc7c6oc6ccccc67)c5)cc4)c4cccc(-c5cccc6c5oc5ccccc56)c4)ccc3C2C=C1]

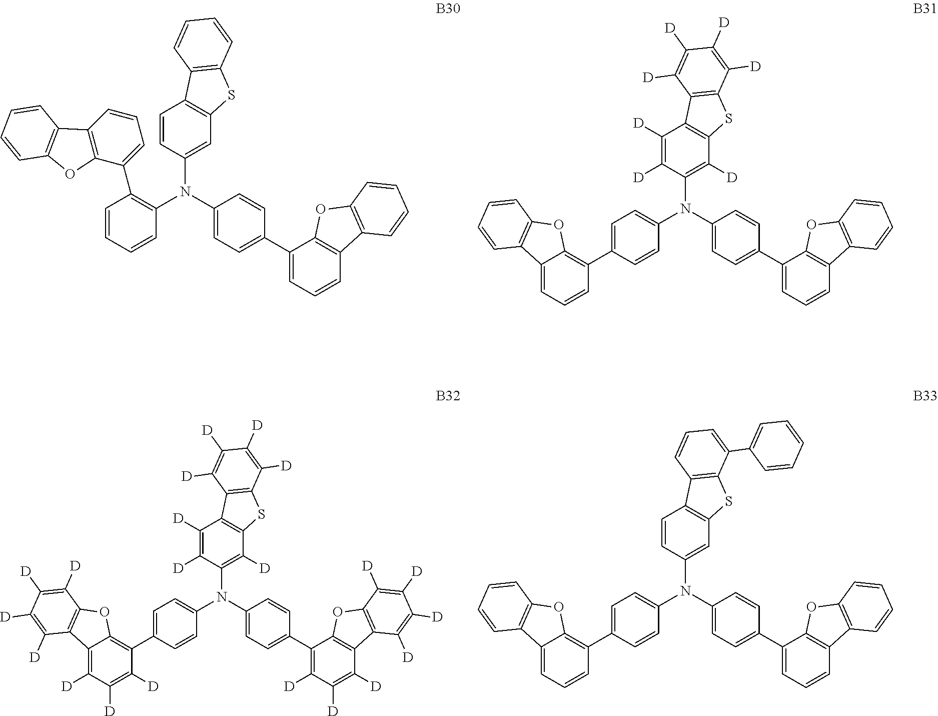

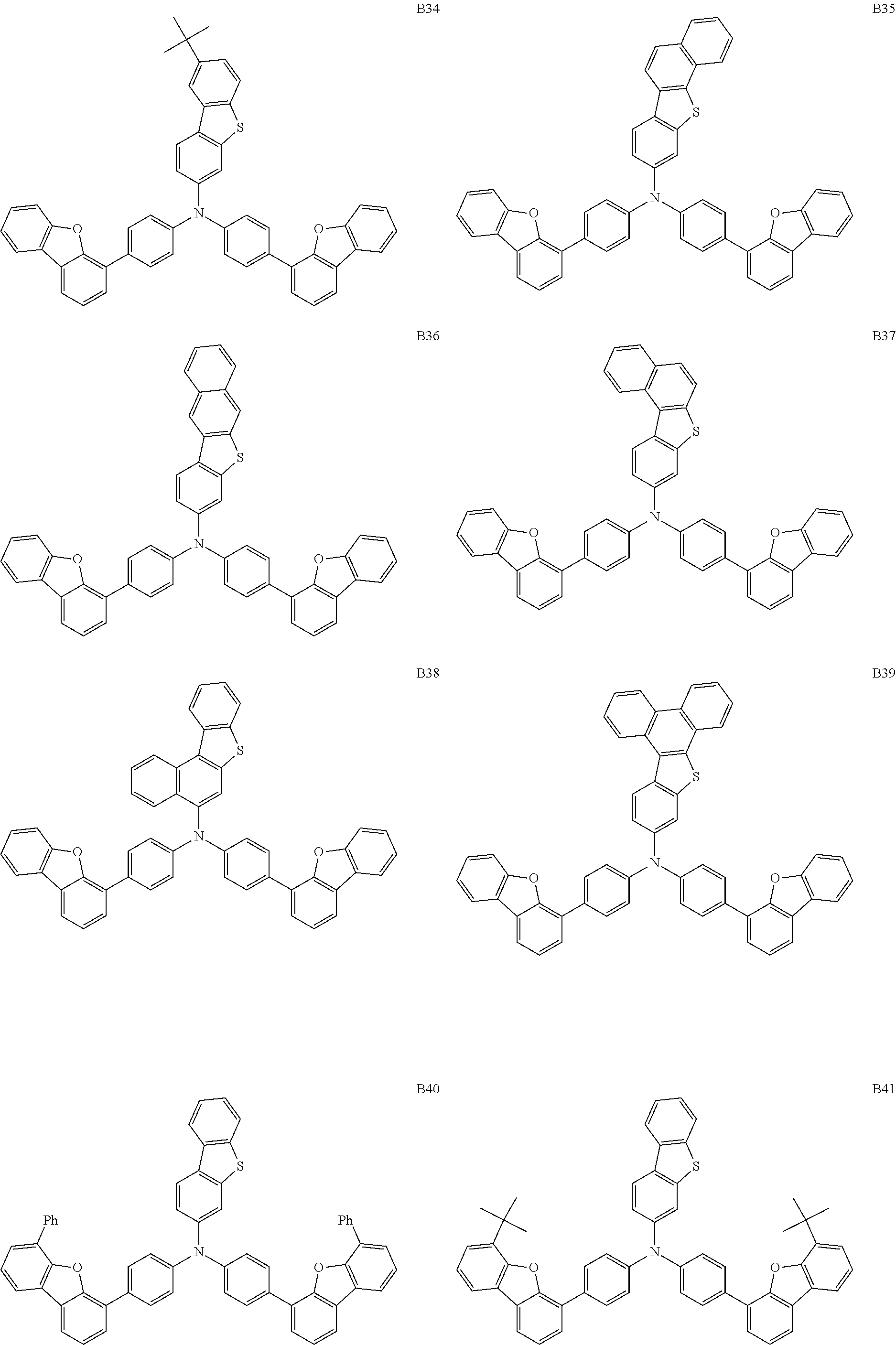

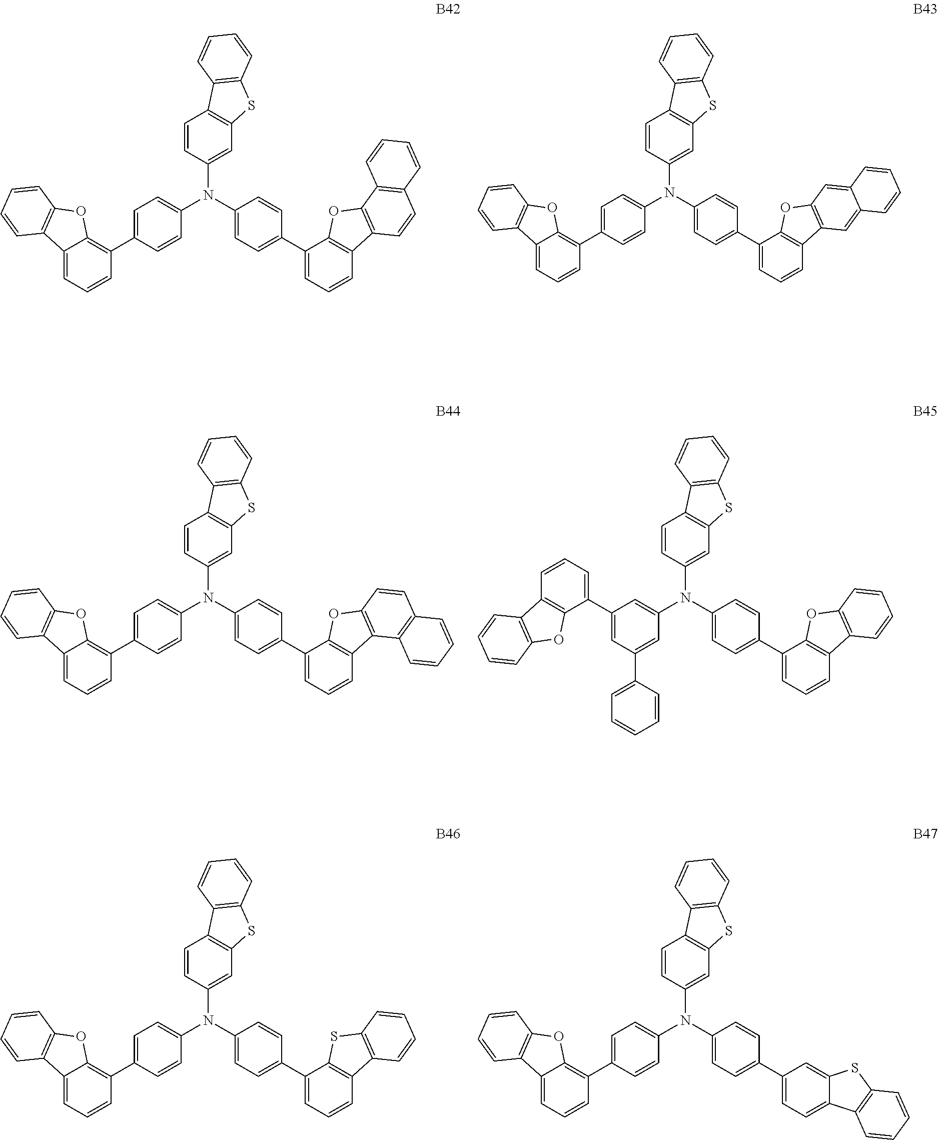

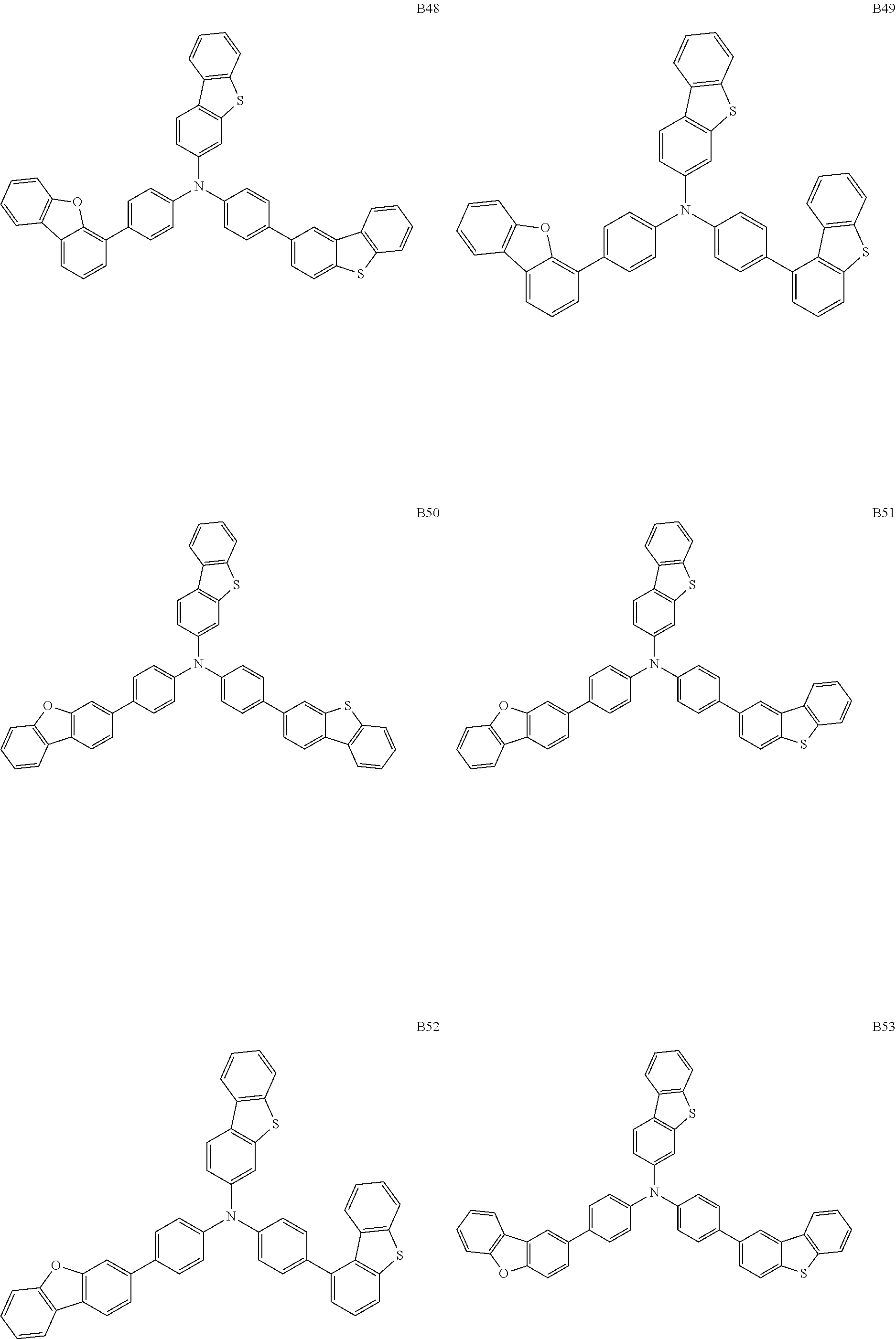

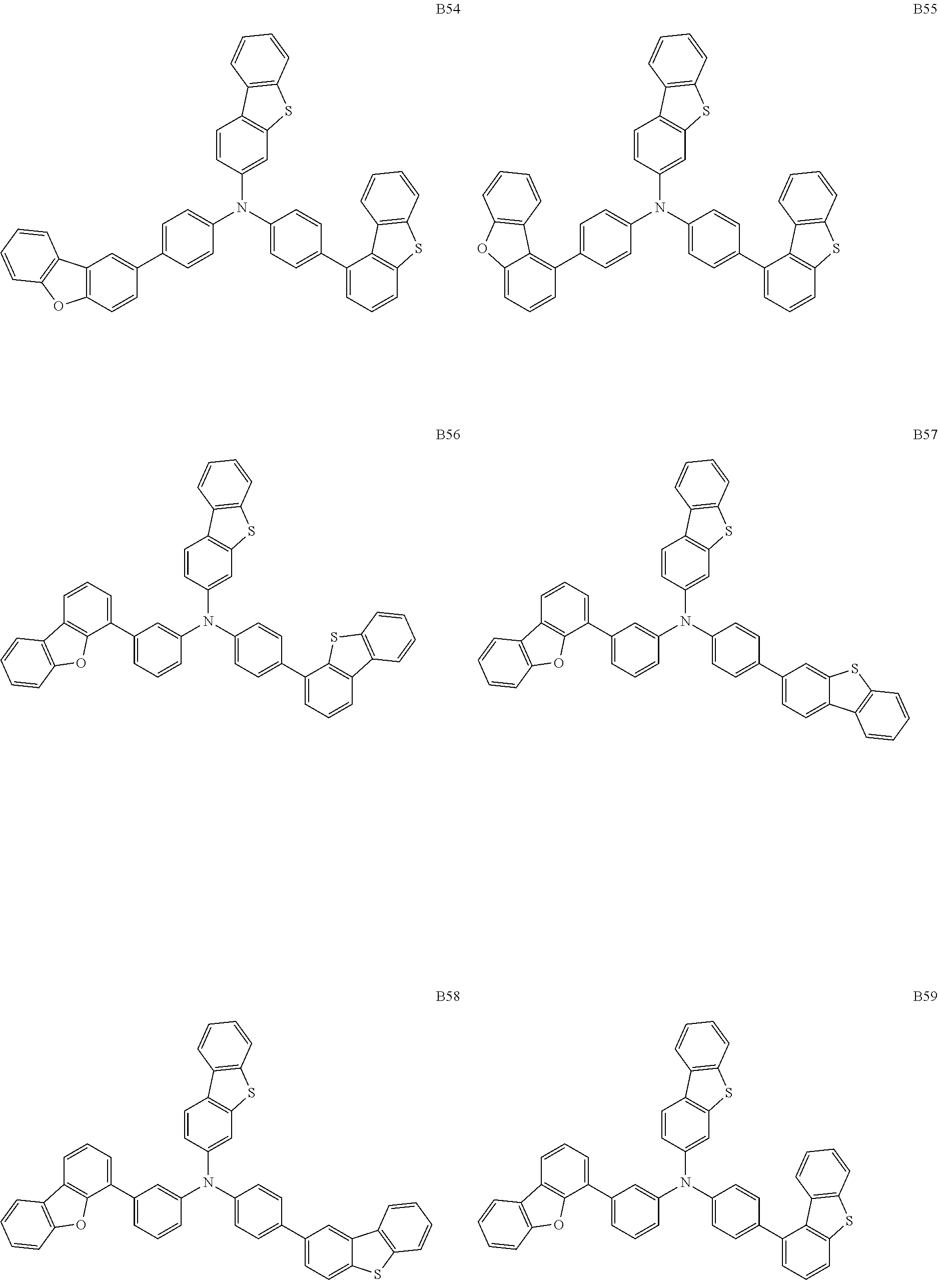

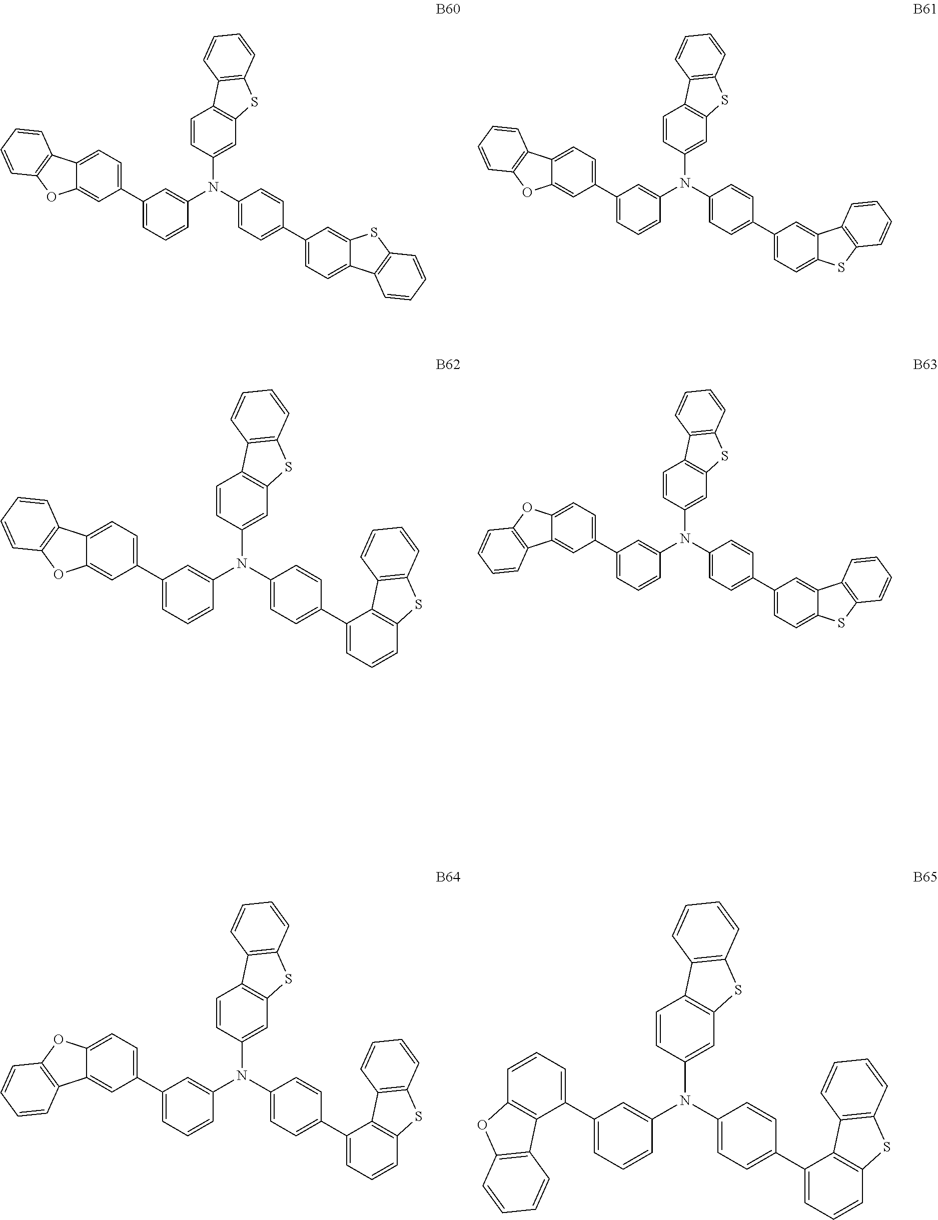

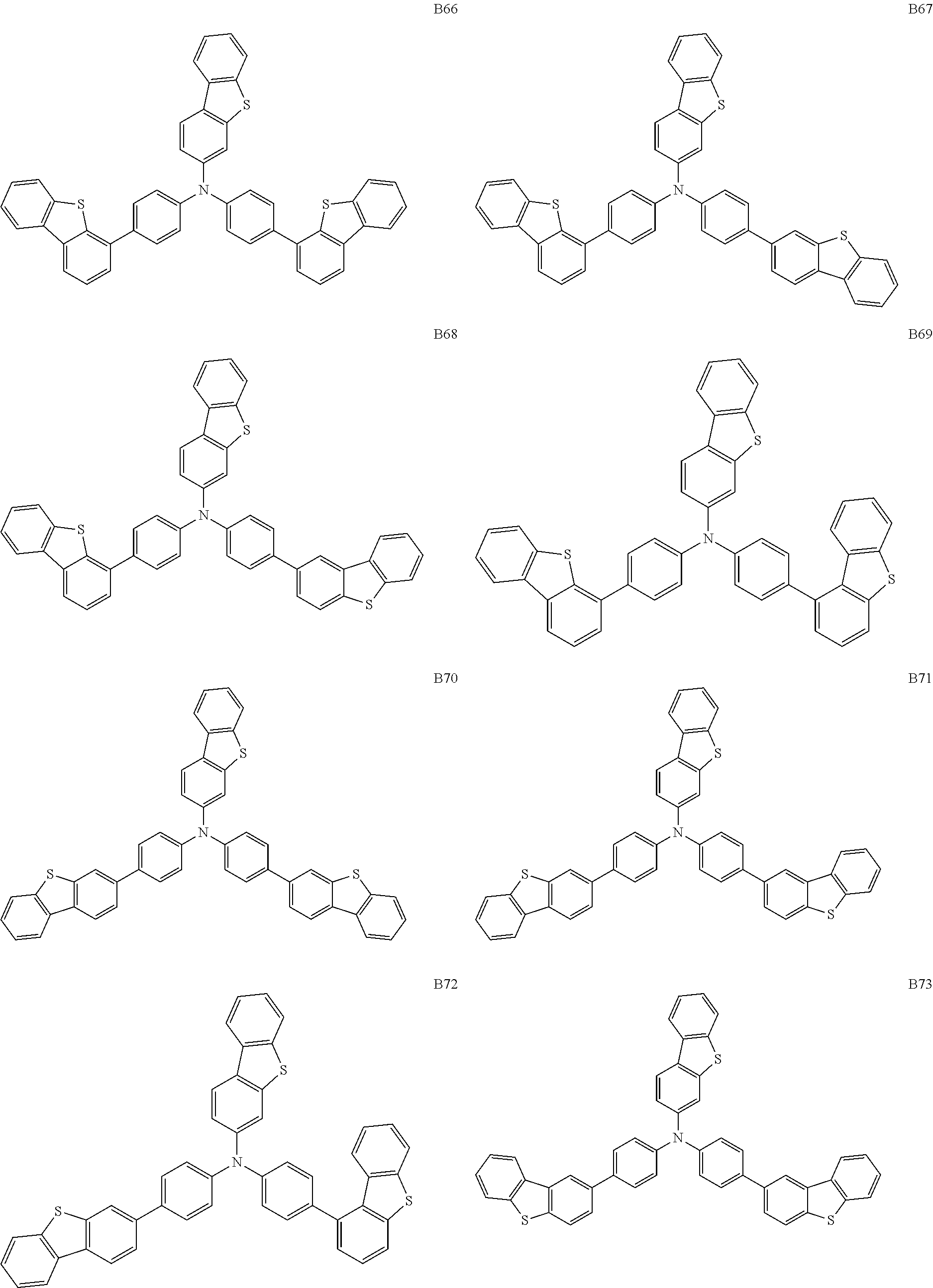

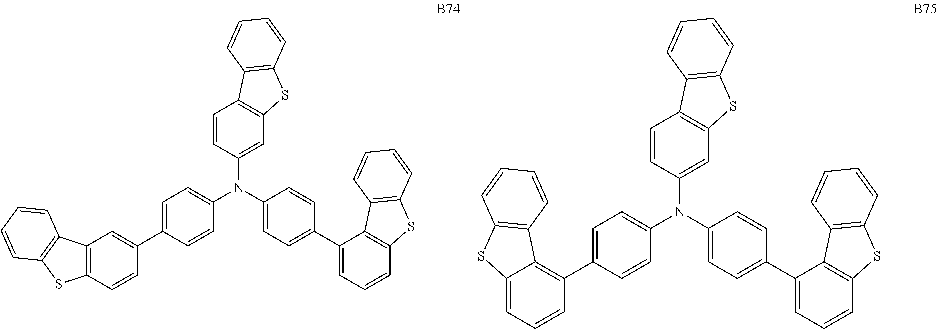 [SMILES: c1ccc2c(c1)sc1cc(N(c3ccc(-c4ccc5sc6ccccc6c5c4)cc3)c3ccc(-c4cccc5sc6ccccc6c45)cc3)ccc12 ; c1ccc2c(c1)sc1cc(N(c3ccc(-c4cccc5sc6ccccc6c45)cc3)c3ccc(-c4cccc5sc6ccccc6c45)cc3)ccc12]

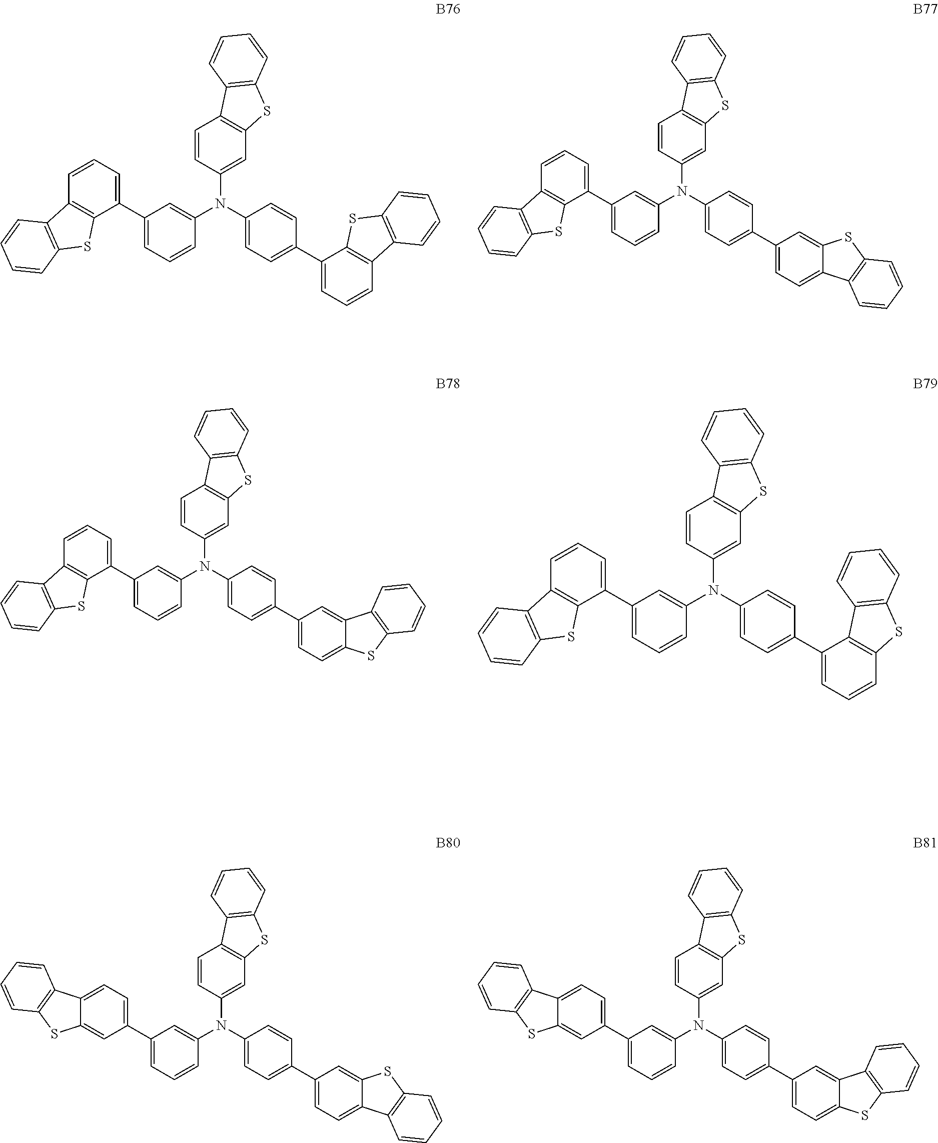

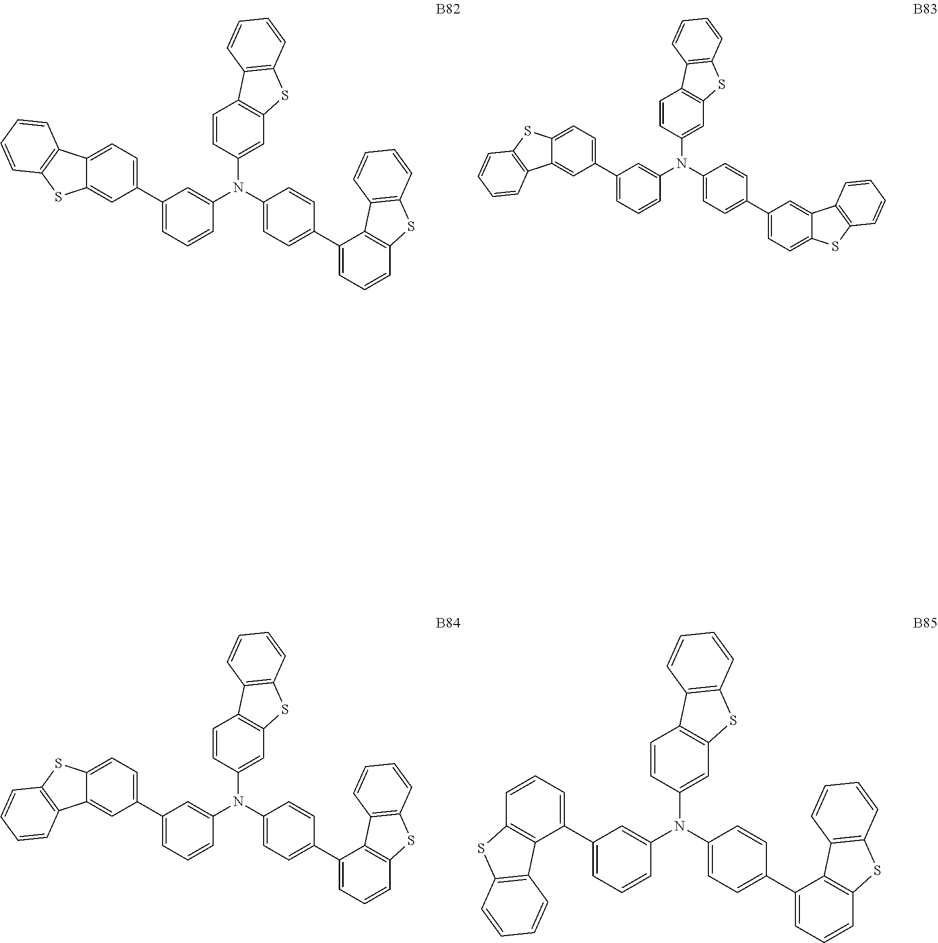 [SMILES: c1cc(-c2ccc3c(c2)sc2ccccc23)cc(N(c2ccc(-c3cccc4sc5ccccc5c34)cc2)c2ccc3c(c2)sc2ccccc23)c1 ; c1cc(-c2ccc3sc4ccccc4c3c2)cc(N(c2ccc(-c3ccc4sc5ccccc5c4c3)cc2)c2ccc3c(c2)sc2ccccc23)c1 ; c1cc(-c2ccc3sc4ccccc4c3c2)cc(N(c2ccc(-c3cccc4sc5ccccc5c34)cc2)c2ccc3c(c2)sc2ccccc23)c1 ; c1cc(-c2cccc3sc4ccccc4c23)cc(N(c2ccc(-c3cccc4sc5ccccc5c34)cc2)c2ccc3c(c2)sc2ccccc23)c1]

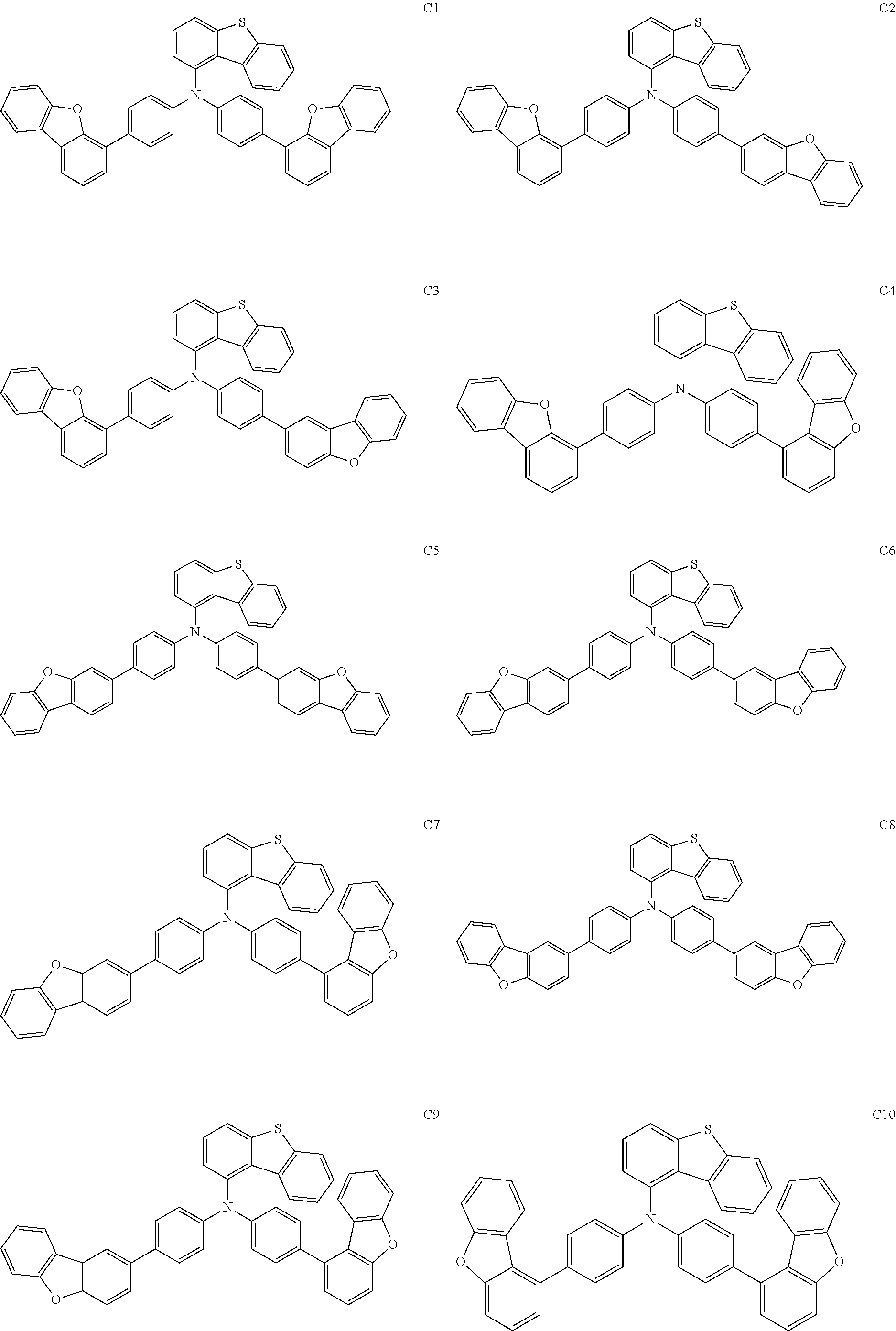

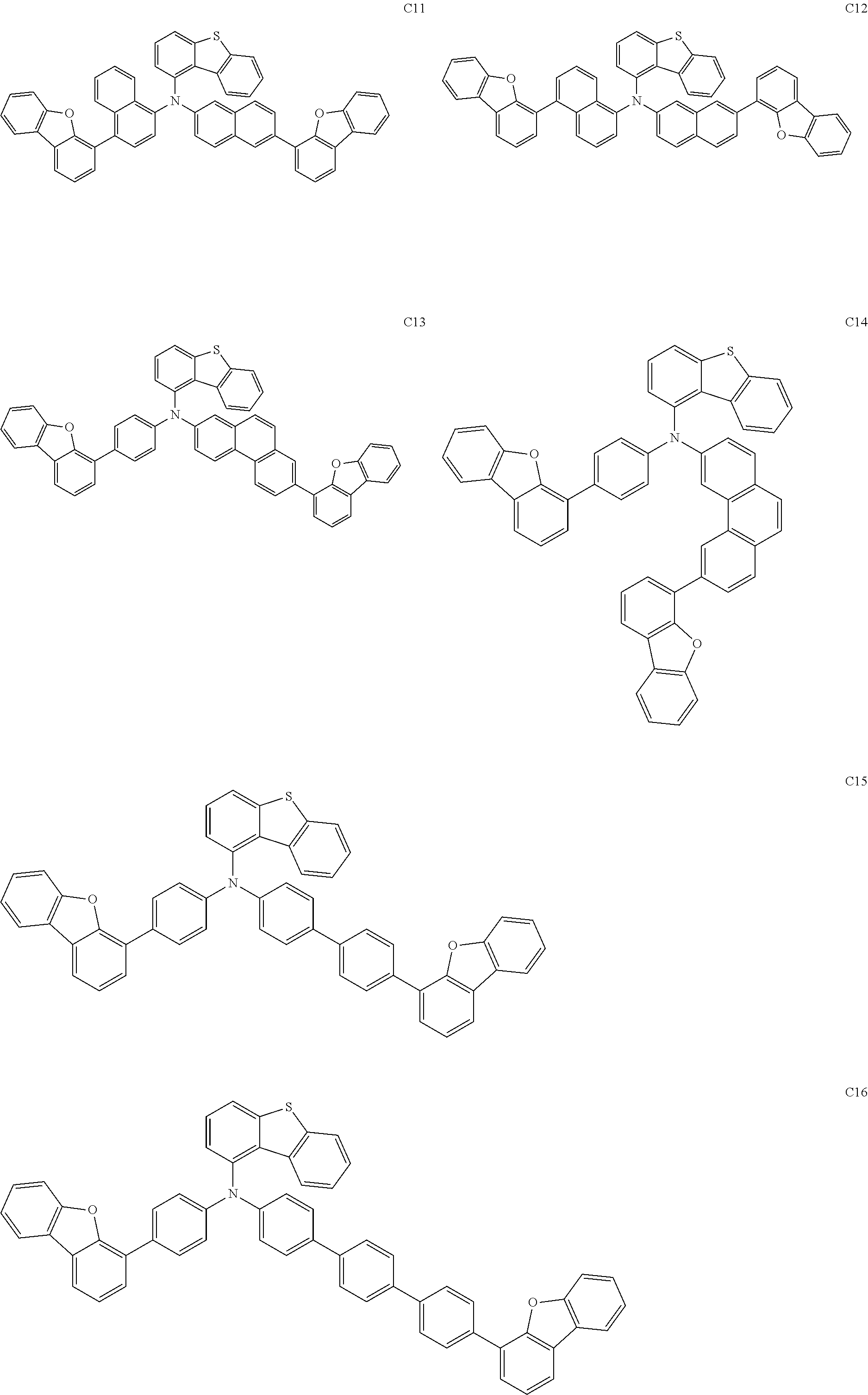

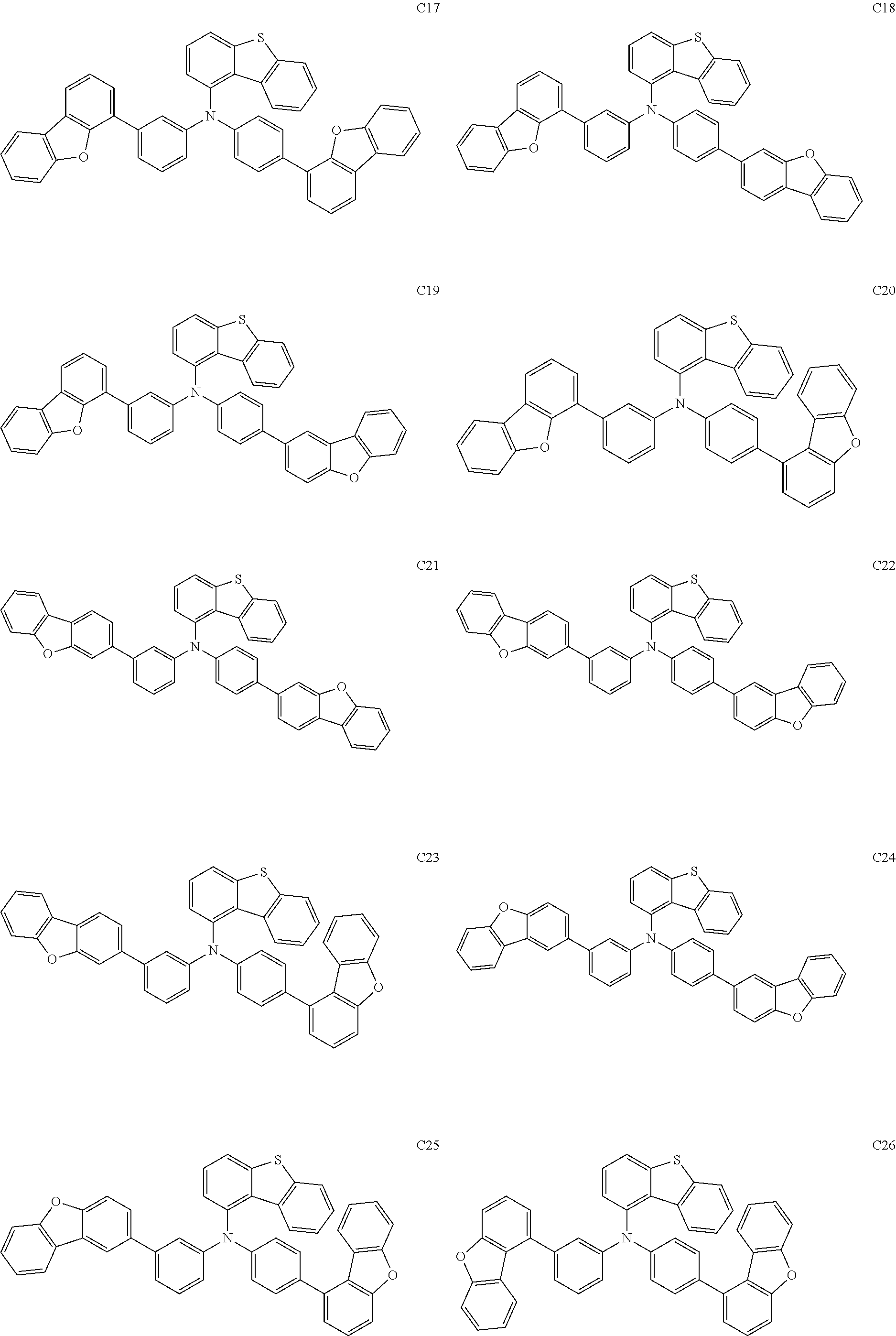

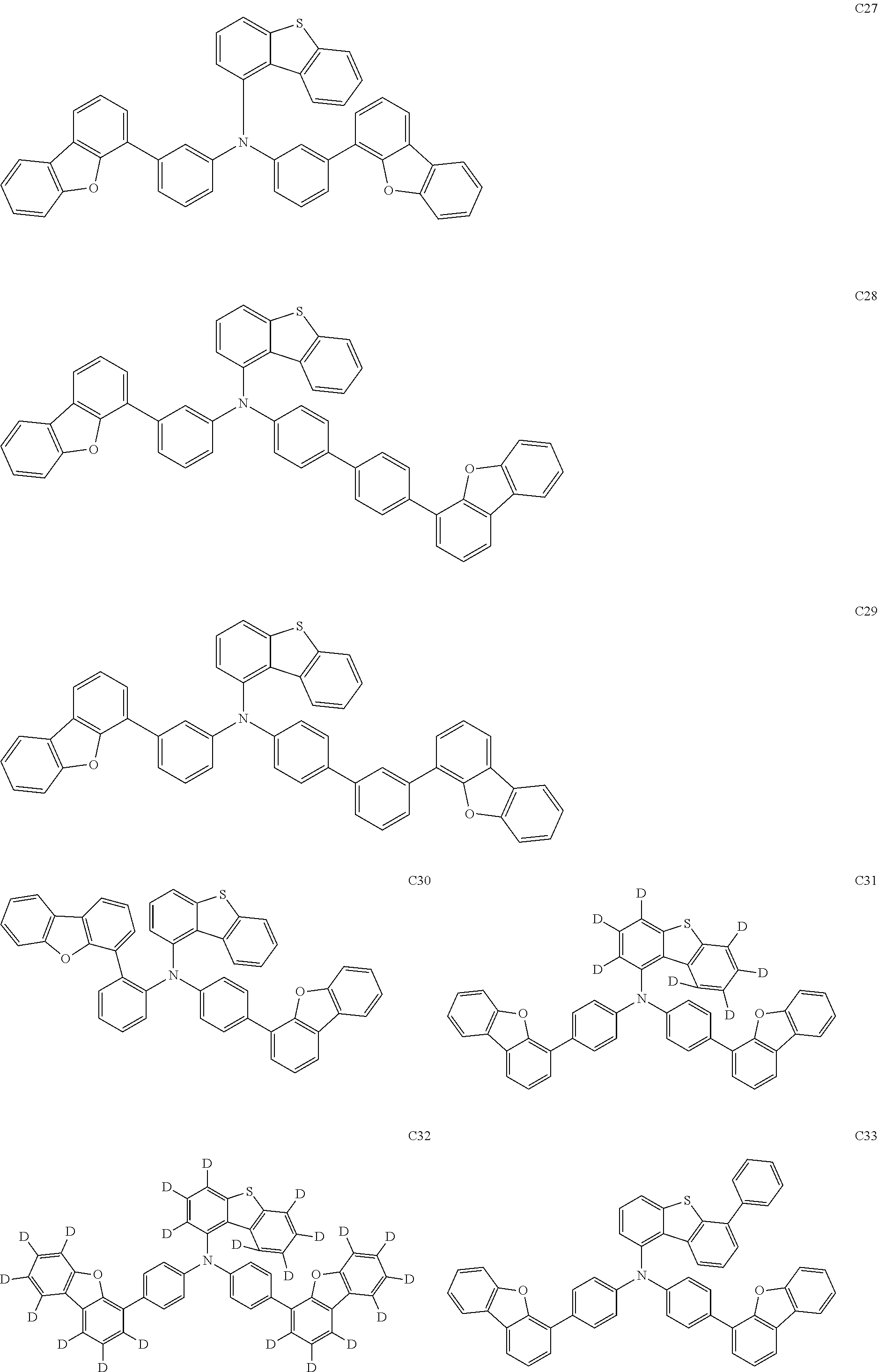

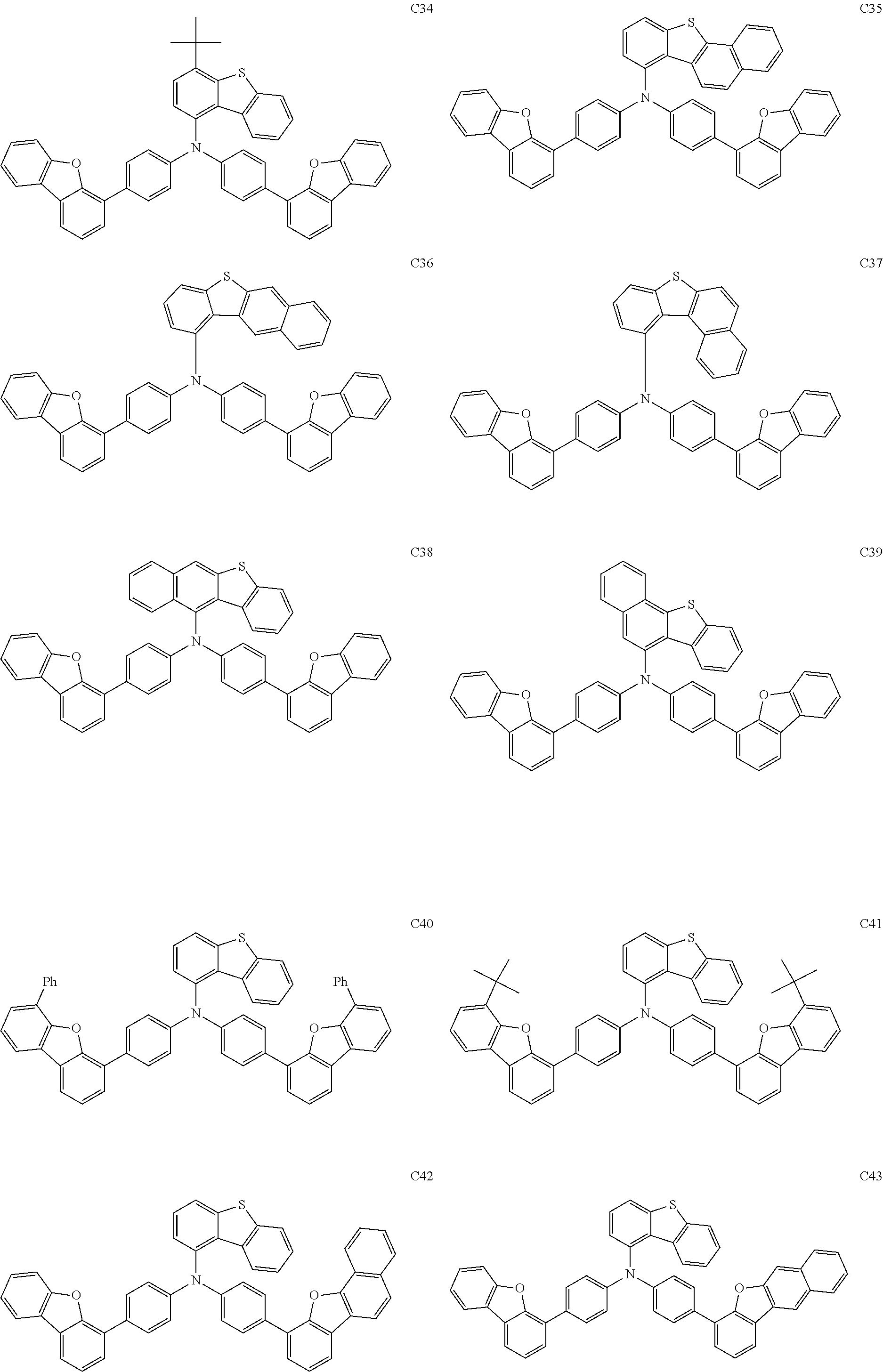

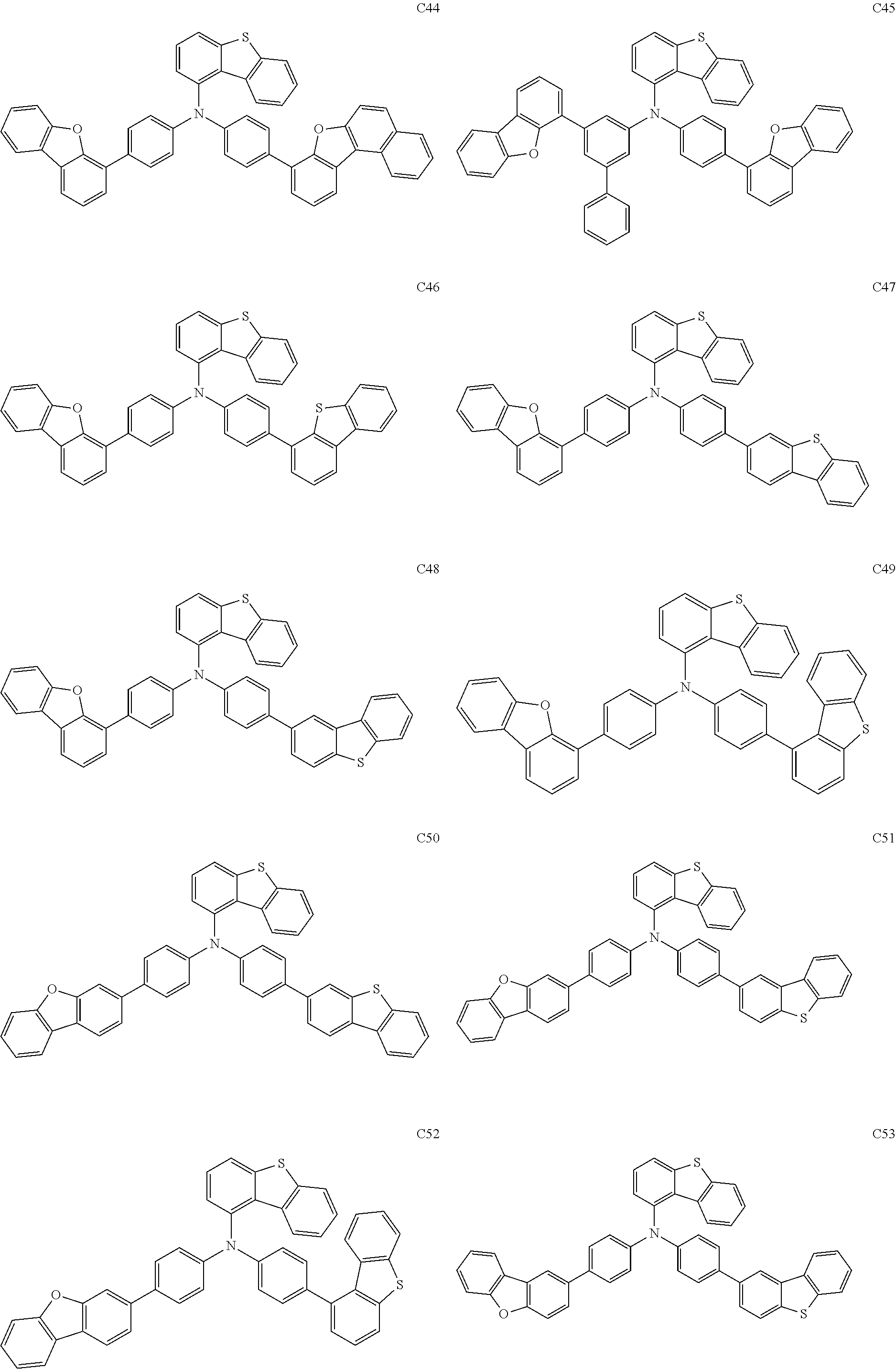

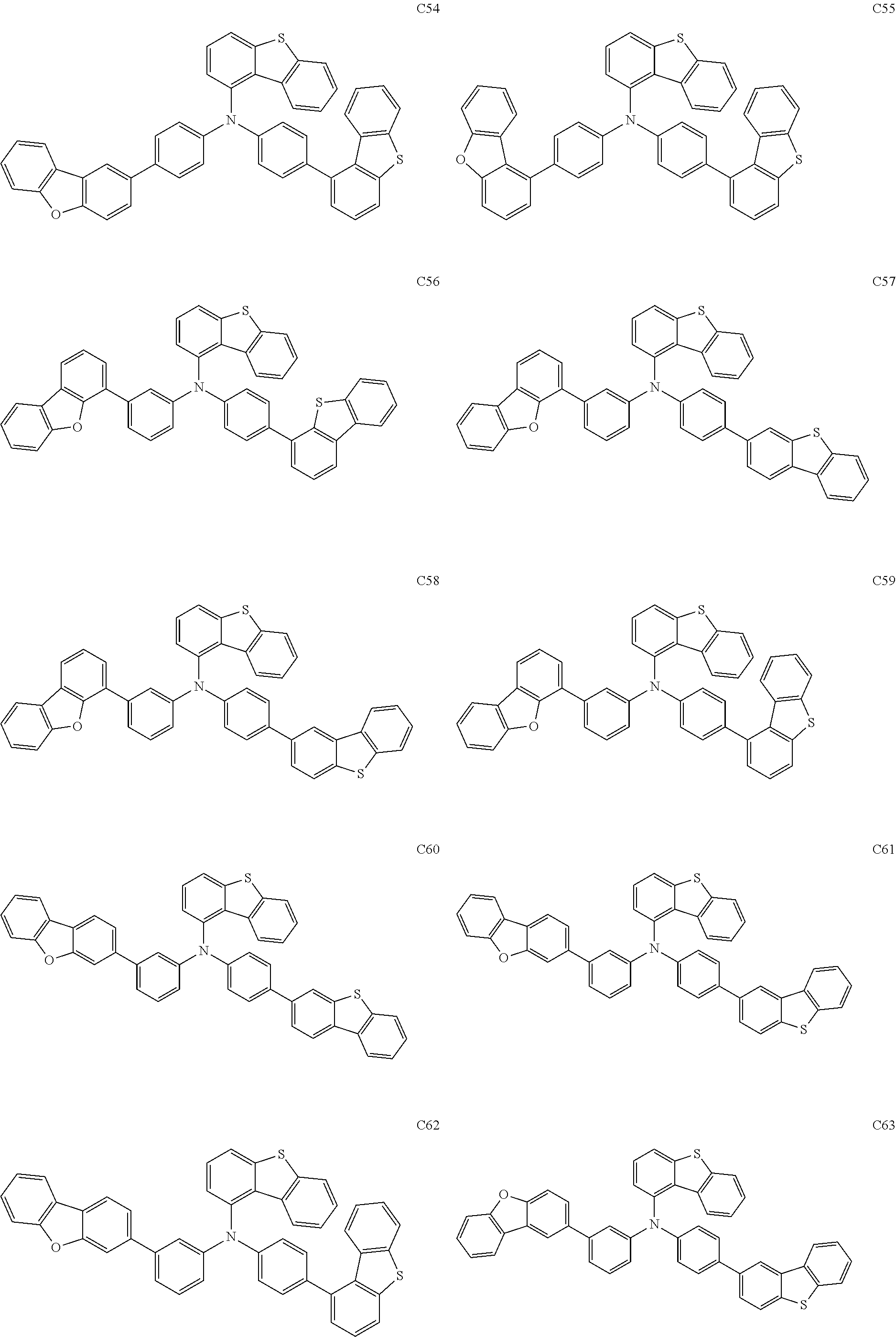

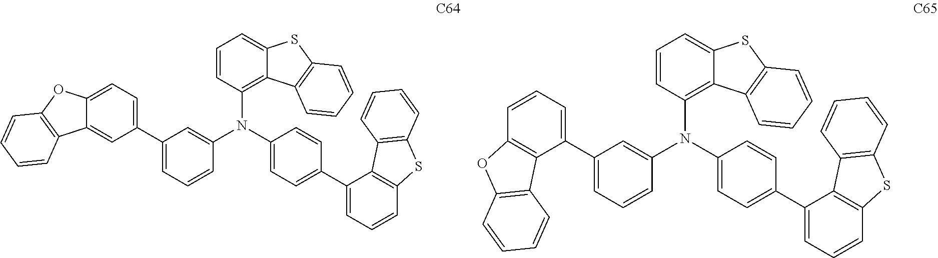 [SMILES: c1cc(-c2ccc3oc4ccccc4c3c2)cc(N(c2ccc(-c3cccc4sc5ccccc5c34)cc2)c2cccc3sc4ccccc4c23)c1 ; c1cc(-c2cccc3oc4ccccc4c23)cc(N(c2ccc(-c3cccc4sc5ccccc5c34)cc2)c2cccc3sc4ccccc4c23)c1]

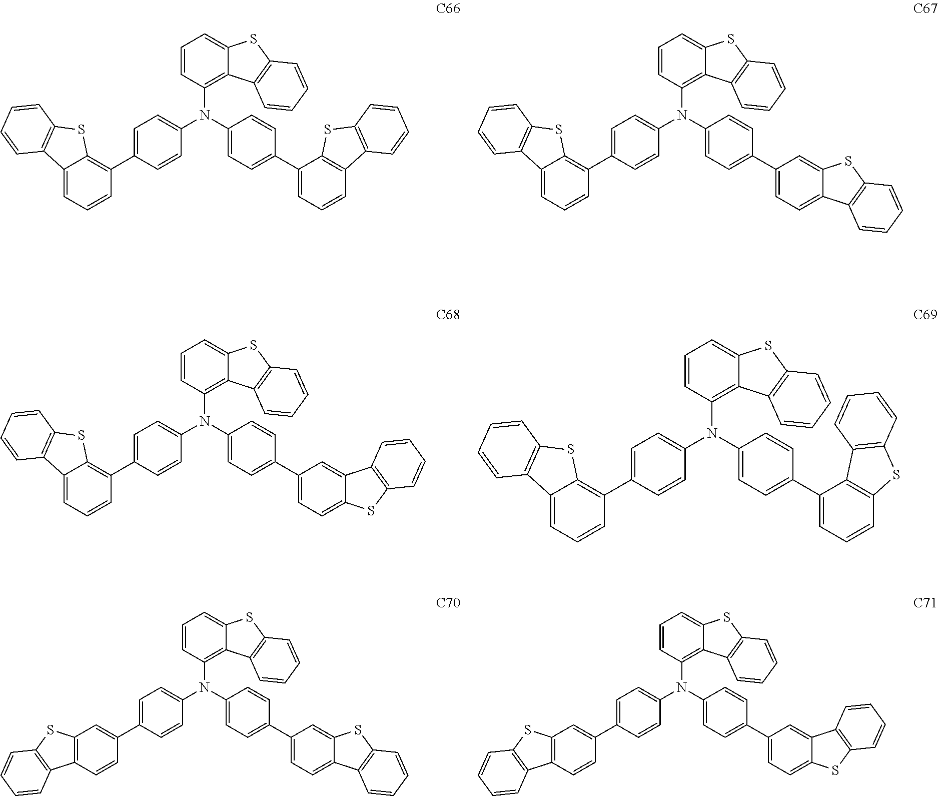

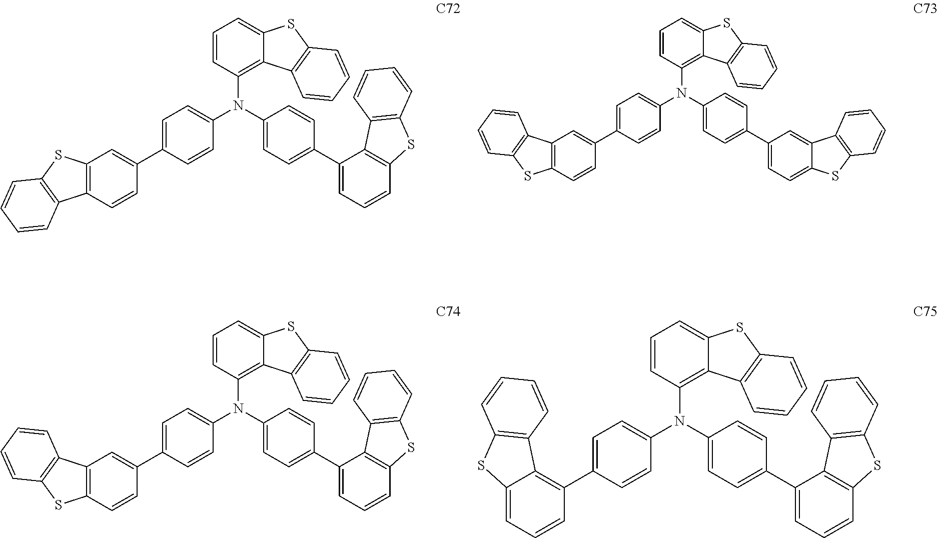 [SMILES: c1ccc2c(c1)sc1cc(-c3ccc(N(c4ccc(-c5cccc6sc7ccccc7c56)cc4)c4cccc5sc6ccccc6c45)cc3)ccc12 ; c1ccc2c(c1)sc1ccc(-c3ccc(N(c4ccc(-c5ccc6sc7ccccc7c6c5)cc4)c4cccc5sc6ccccc6c45)cc3)cc12 ; c1ccc2c(c1)sc1ccc(-c3ccc(N(c4ccc(-c5cccc6sc7ccccc7c56)cc4)c4cccc5sc6ccccc6c45)cc3)cc12 ; c1ccc2c(c1)sc1cccc(-c3ccc(N(c4ccc(-c5cccc6sc7ccccc7c56)cc4)c4cccc5sc6ccccc6c45)cc3)c12]

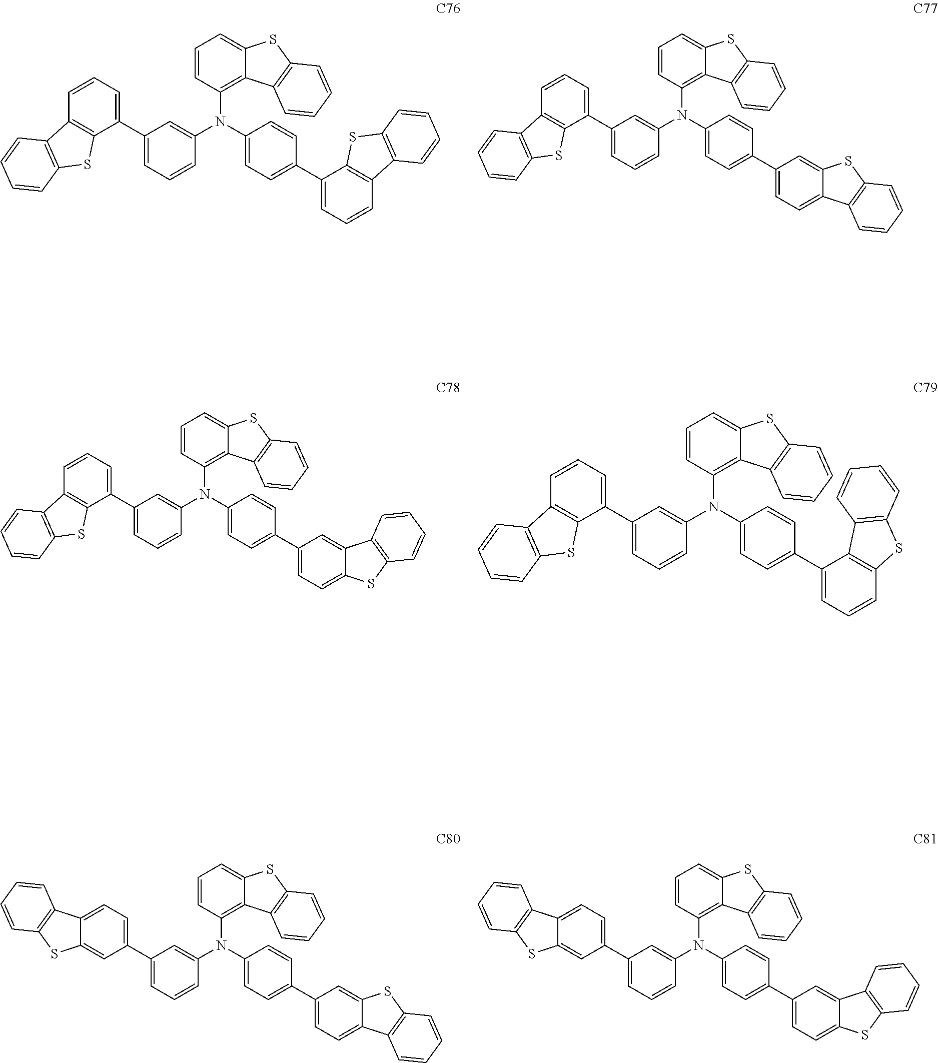

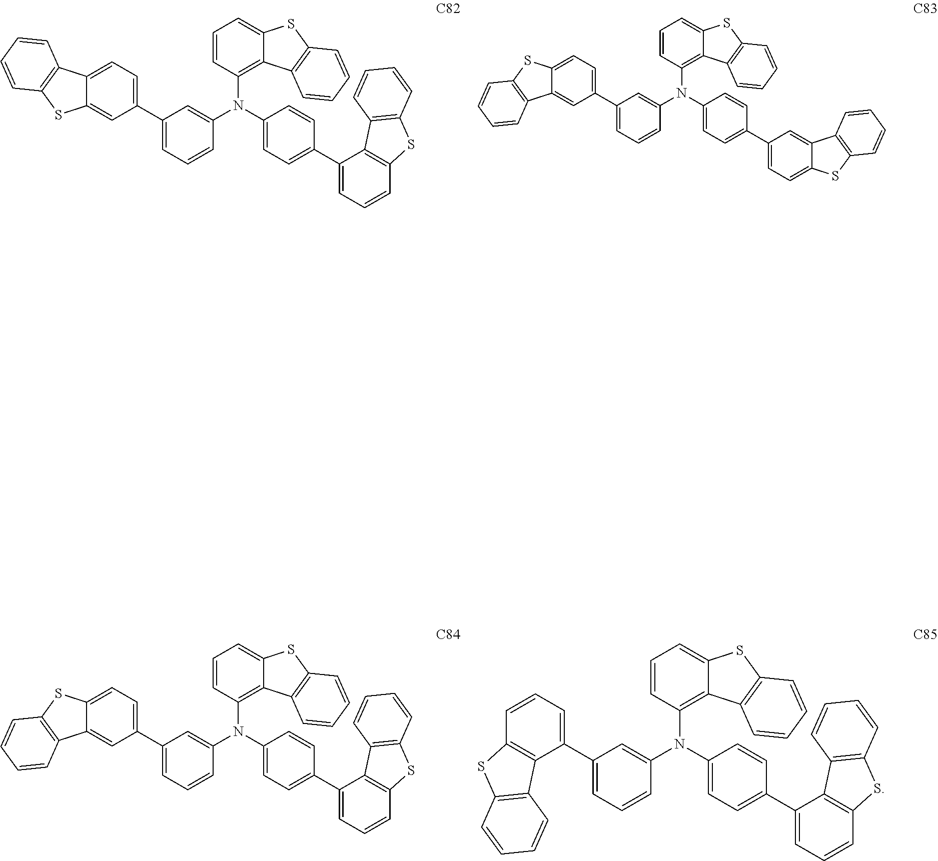 [SMILES: c1cc(-c2ccc3c(c2)sc2ccccc23)cc(N(c2ccc(-c3cccc4sc5ccccc5c34)cc2)c2cccc3sc4ccccc4c23)c1 ; c1cc(-c2ccc3sc4ccccc4c3c2)cc(N(c2ccc(-c3ccc4sc5ccccc5c4c3)cc2)c2cccc3sc4ccccc4c23)c1 ; c1cc(-c2ccc3sc4ccccc4c3c2)cc(N(c2ccc(-c3cccc4sc5ccccc5c34)cc2)c2cccc3sc4ccccc4c23)c1 ; c1cc(-c2cccc3sc4ccccc4c23)cc(N(c2ccc(-c3cccc4sc5ccccc5c34)cc2)c2cccc3sc4ccccc4c23)c1]

11. The luminescence device of claim 1, wherein the amine compound represented by Formula 1 is at least one selected from Compound Group 2:

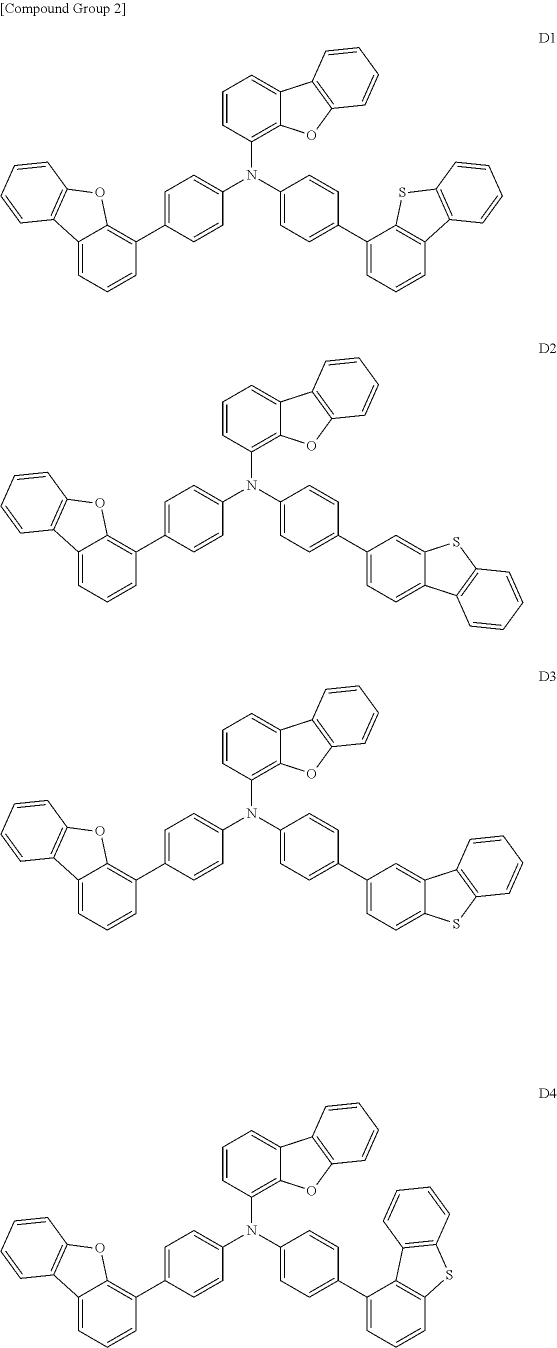 [SMILES: c1ccc2c(c1)oc1c(-c3ccc(N(c4ccc(-c5cccc6c5sc5ccccc56)cc4)c4cccc5c4oc4ccccc45)cc3)cccc12 ; c1ccc2c(c1)oc1c(-c3ccc(N(c4ccc(-c5ccc6c(c5)sc5ccccc56)cc4)c4cccc5c4oc4ccccc45)cc3)cccc12 ; c1ccc2c(c1)oc1c(-c3ccc(N(c4ccc(-c5ccc6sc7ccccc7c6c5)cc4)c4cccc5c4oc4ccccc45)cc3)cccc12 ; c1ccc2c(c1)oc1c(-c3ccc(N(c4ccc(-c5cccc6sc7ccccc7c56)cc4)c4cccc5c4oc4ccccc45)cc3)cccc12]

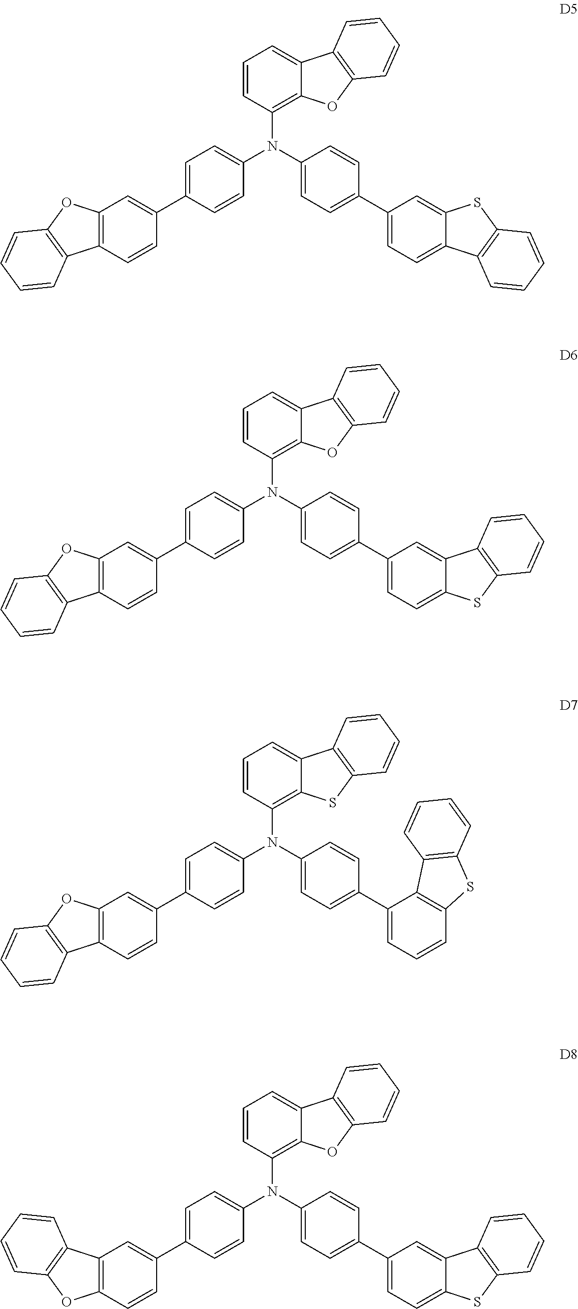 [SMILES: c1ccc2c(c1)oc1cc(-c3ccc(N(c4ccc(-c5ccc6c(c5)sc5ccccc56)cc4)c4cccc5c4oc4ccccc45)cc3)ccc12 ; c1ccc2c(c1)oc1cc(-c3ccc(N(c4ccc(-c5ccc6sc7ccccc7c6c5)cc4)c4cccc5c4oc4ccccc45)cc3)ccc12 ; c1ccc2c(c1)oc1cc(-c3ccc(N(c4ccc(-c5cccc6sc7ccccc7c56)cc4)c4cccc5c4sc4ccccc45)cc3)ccc12 ; c1ccc2c(c1)oc1ccc(-c3ccc(N(c4ccc(-c5ccc6sc7ccccc7c6c5)cc4)c4cccc5c4oc4ccccc45)cc3)cc12]

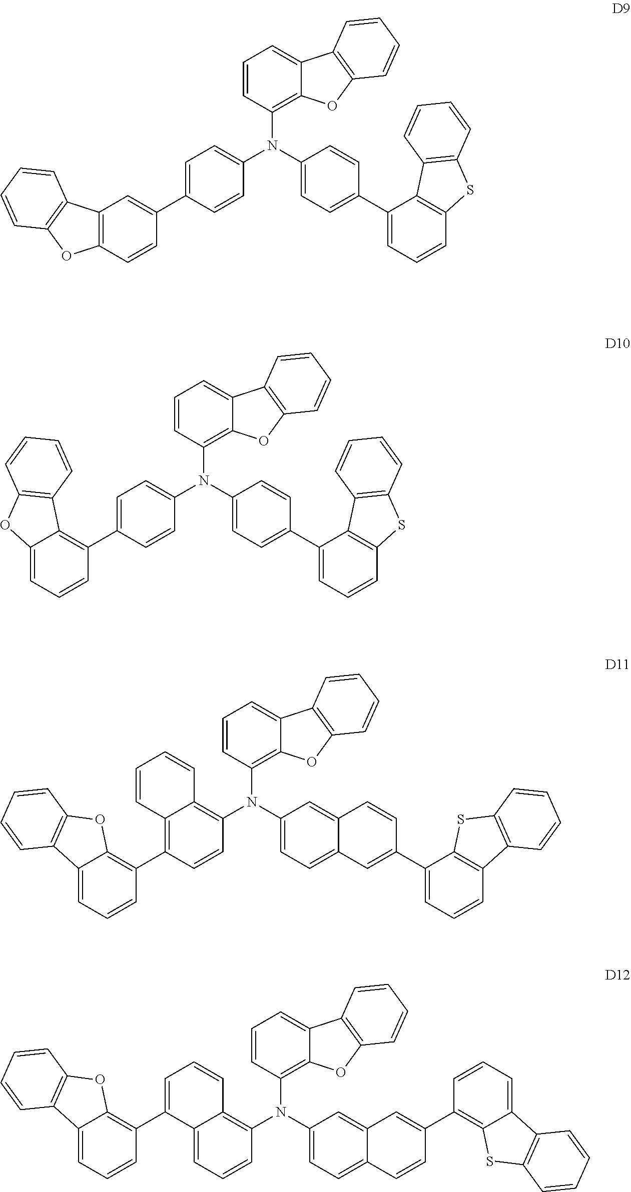 [SMILES: c1ccc2c(c1)oc1ccc(-c3ccc(N(c4ccc(-c5cccc6sc7ccccc7c56)cc4)c4cccc5c4oc4ccccc45)cc3)cc12 ; c1ccc2c(c1)oc1c(N(c3ccc(-c4cccc5oc6ccccc6c45)cc3)c3ccc(-c4cccc5sc6ccccc6c45)cc3)cccc12 ; c1ccc2c(c1)oc1c(-c3ccc(N(c4ccc5cc(-c6cccc7c6sc6ccccc67)ccc5c4)c4cccc5c4oc4ccccc45)c4ccccc34)cccc12 ; c1ccc2c(c1)oc1c(-c3cccc4c(N(c5ccc6ccc(-c7cccc8c7sc7ccccc78)cc6c5)c5cccc6c5oc5ccccc56)cccc34)cccc12]

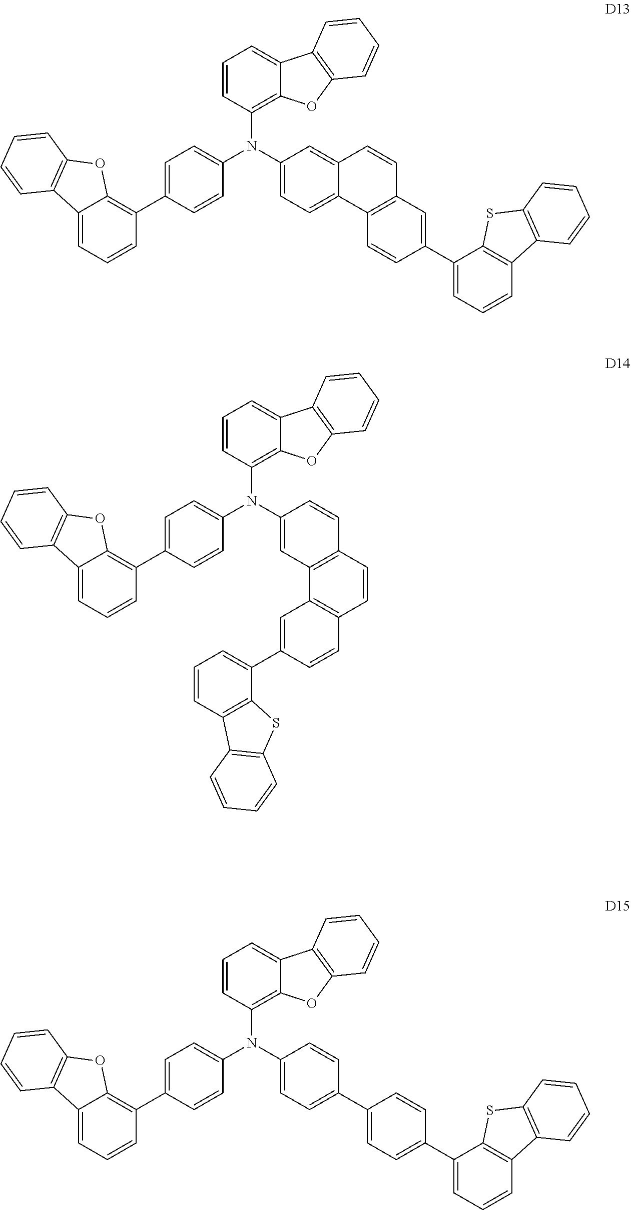 [SMILES: c1ccc2c(c1)oc1c(-c3ccc(N(c4ccc5c(ccc6cc(-c7cccc8c7sc7ccccc78)ccc65)c4)c4cccc5c4oc4ccccc45)cc3)cccc12 ; c1ccc2c(c1)oc1c(-c3ccc(N(c4ccc5ccc6ccc(-c7cccc8c7sc7ccccc78)cc6c5c4)c4cccc5c4oc4ccccc45)cc3)cccc12 ; c1ccc2c(c1)oc1c(-c3ccc(N(c4ccc(-c5ccc(-c6cccc7c6sc6ccccc67)cc5)cc4)c4cccc5c4oc4ccccc45)cc3)cccc12]

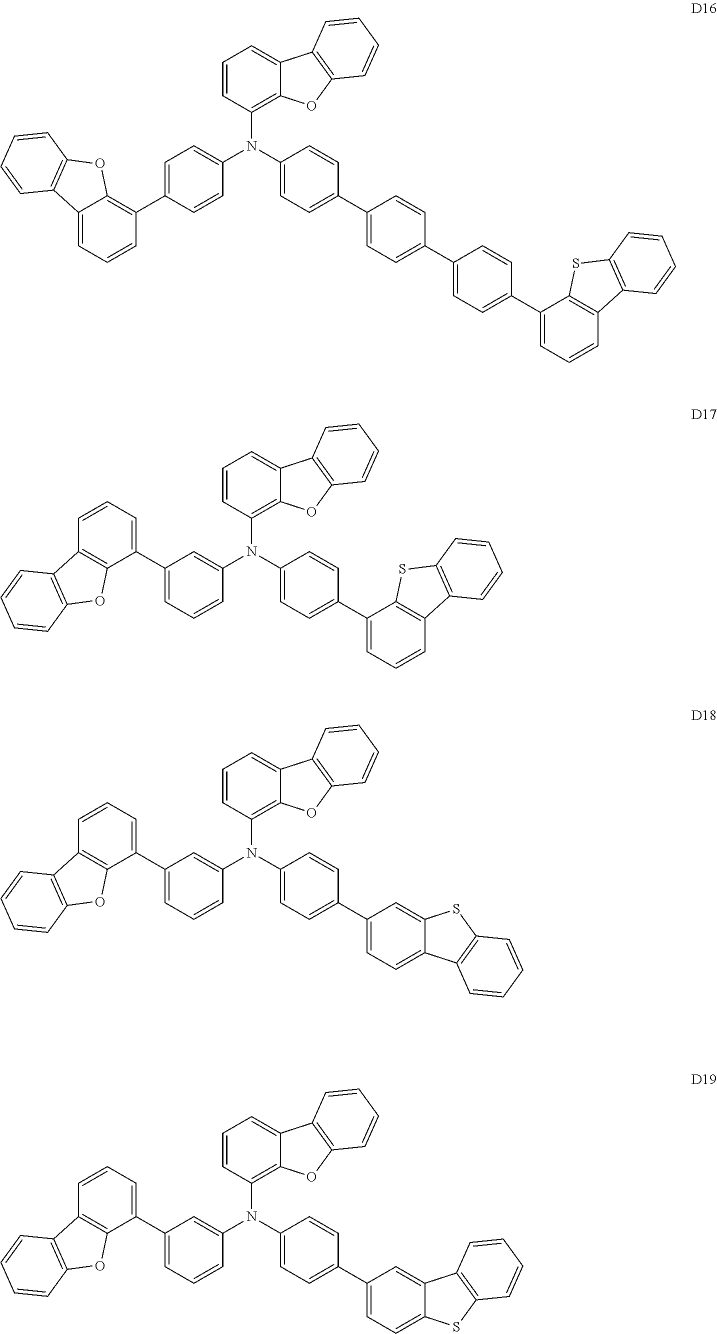 [SMILES: c1ccc2c(c1)oc1c(-c3ccc(N(c4ccc(-c5ccc(-c6ccc(-c7cccc8c7sc7ccccc78)cc6)cc5)cc4)c4cccc5c4oc4ccccc45)cc3)cccc12 ; c1cc(-c2cccc3c2oc2ccccc23)cc(N(c2ccc(-c3cccc4c3sc3ccccc34)cc2)c2cccc3c2oc2ccccc23)c1 ; c1cc(-c2cccc3c2oc2ccccc23)cc(N(c2ccc(-c3ccc4c(c3)sc3ccccc34)cc2)c2cccc3c2oc2ccccc23)c1 ; c1cc(-c2cccc3c2oc2ccccc23)cc(N(c2ccc(-c3ccc4sc5ccccc5c4c3)cc2)c2cccc3c2oc2ccccc23)c1]

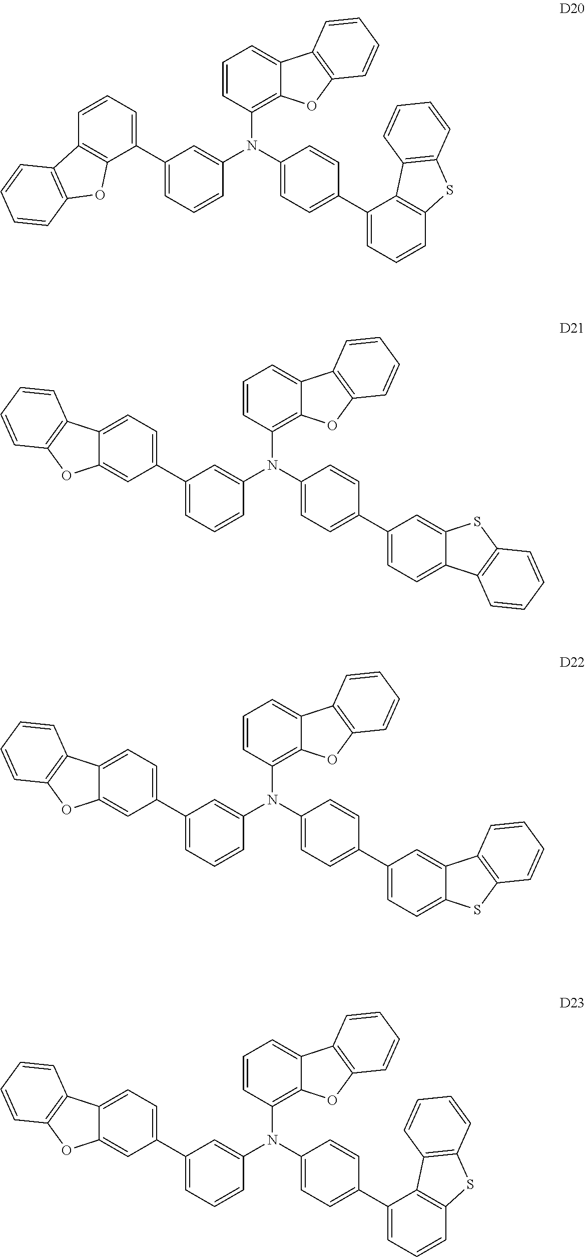 [SMILES: c1cc(-c2cccc3c2oc2ccccc23)cc(N(c2ccc(-c3cccc4sc5ccccc5c34)cc2)c2cccc3c2oc2ccccc23)c1 ; c1cc(-c2ccc3c(c2)oc2ccccc23)cc(N(c2ccc(-c3ccc4c(c3)sc3ccccc34)cc2)c2cccc3c2oc2ccccc23)c1 ; c1cc(-c2ccc3c(c2)oc2ccccc23)cc(N(c2ccc(-c3ccc4sc5ccccc5c4c3)cc2)c2cccc3c2oc2ccccc23)c1 ; c1cc(-c2ccc3c(c2)oc2ccccc23)cc(N(c2ccc(-c3cccc4sc5ccccc5c34)cc2)c2cccc3c2oc2ccccc23)c1]

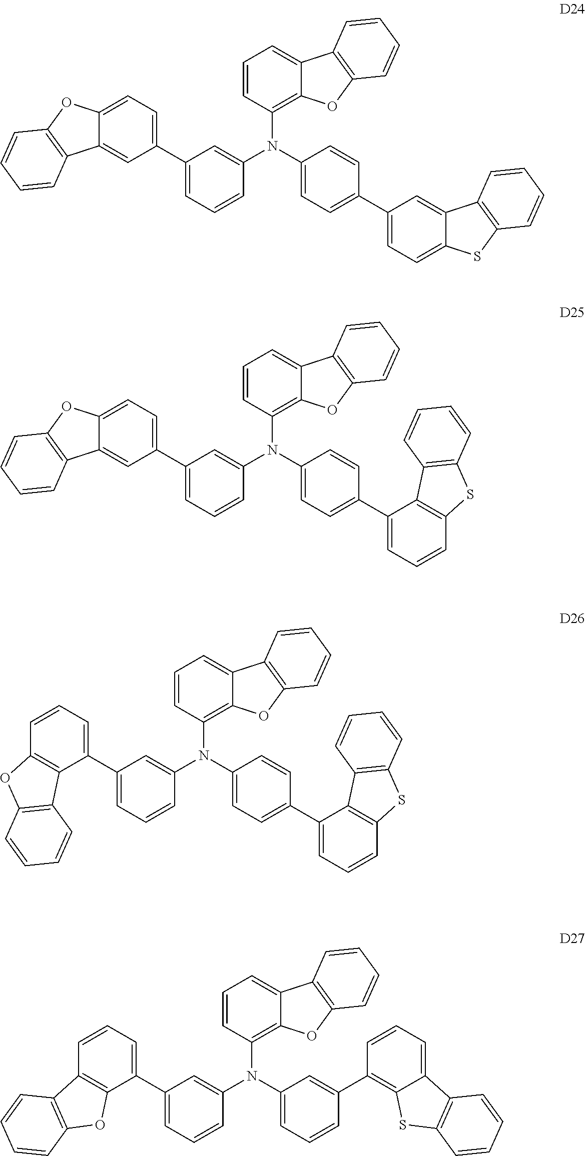 [SMILES: c1cc(-c2ccc3oc4ccccc4c3c2)cc(N(c2ccc(-c3ccc4sc5ccccc5c4c3)cc2)c2cccc3c2oc2ccccc23)c1 ; c1cc(-c2ccc3oc4ccccc4c3c2)cc(N(c2ccc(-c3cccc4sc5ccccc5c34)cc2)c2cccc3c2oc2ccccc23)c1 ; c1cc(-c2cccc3oc4ccccc4c23)cc(N(c2ccc(-c3cccc4sc5ccccc5c34)cc2)c2cccc3c2oc2ccccc23)c1 ; c1cc(-c2cccc3c2oc2ccccc23)cc(N(c2cccc(-c3cccc4c3sc3ccccc34)c2)c2cccc3c2oc2ccccc23)c1]

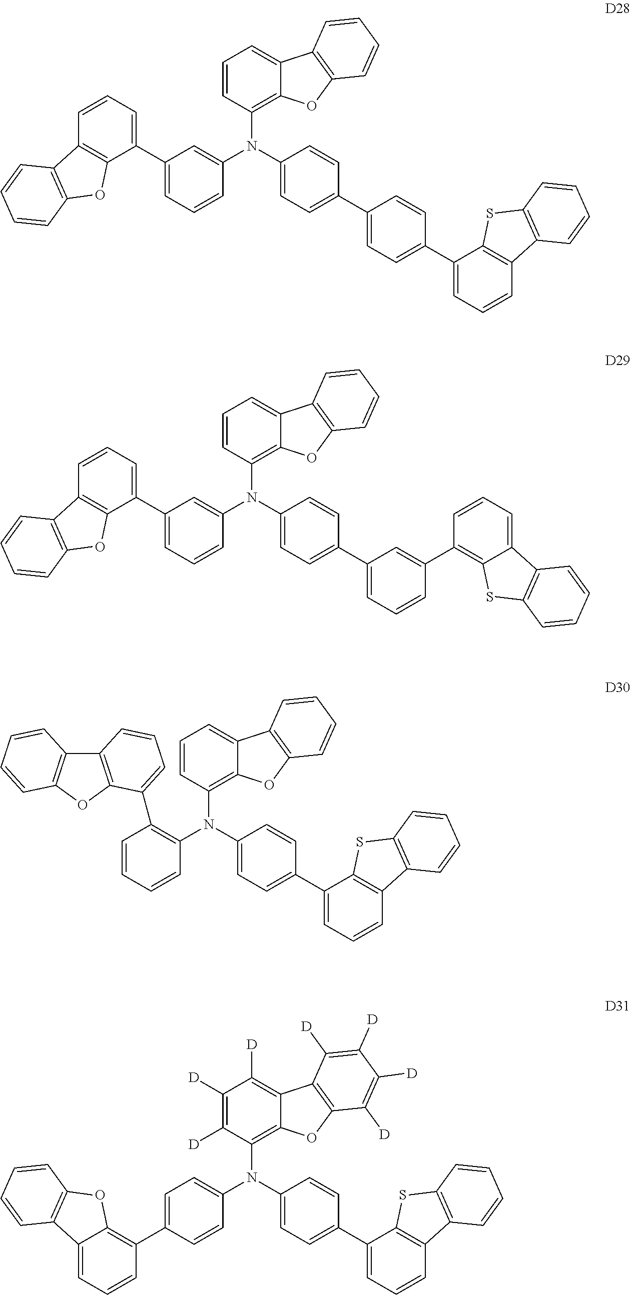 [SMILES: c1cc(-c2cccc3c2oc2ccccc23)cc(N(c2ccc(-c3ccc(-c4cccc5c4sc4ccccc45)cc3)cc2)c2cccc3c2oc2ccccc23)c1 ; c1cc(-c2ccc(N(c3cccc(-c4cccc5c4oc4ccccc45)c3)c3cccc4c3oc3ccccc34)cc2)cc(-c2cccc3c2sc2ccccc23)c1 ; c1ccc(N(c2ccc(-c3cccc4c3sc3ccccc34)cc2)c2cccc3c2oc2ccccc23)c(-c2cccc3c2oc2ccccc23)c1 ; [2H]c1c([2H])c([2H])c2c(oc3c(N(c4ccc(-c5cccc6c5oc5ccccc56)cc4)c4ccc(-c5cccc6c5sc5ccccc56)cc4)c([2H])c([2H])c([2H])c32)c1[2H]]

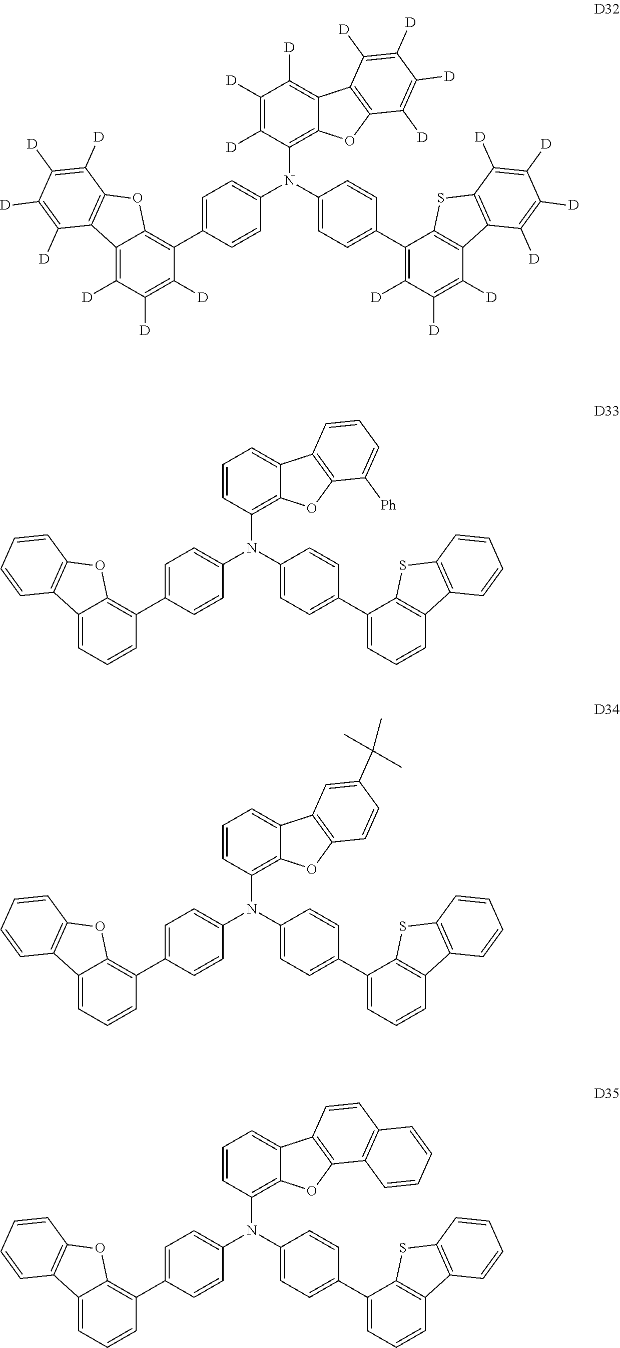 [SMILES: [2H]c1c([2H])c([2H])c2c(oc3c(-c4ccc(N(c5ccc(-c6c([2H])c([2H])c([2H])c7c6sc6c([2H])c([2H])c([2H])c([2H])c67)cc5)c5c([2H])c([2H])c([2H])c6c5oc5c([2H])c([2H])c([2H])c([2H])c56)cc4)c([2H])c([2H])c([2H])c32)c1[2H] ; c1ccc(-c2cccc3c2oc2c(N(c4ccc(-c5cccc6c5oc5ccccc56)cc4)c4ccc(-c5cccc6c5sc5ccccc56)cc4)cccc23)cc1 ; CC(C)(C)c1ccc2oc3c(N(c4ccc(-c5cccc6c5oc5ccccc56)cc4)c4ccc(-c5cccc6c5sc5ccccc56)cc4)cccc3c2c1 ; c1ccc2c(c1)ccc1c3cccc(N(c4ccc(-c5cccc6c5oc5ccccc56)cc4)c4ccc(-c5cccc6c5sc5ccccc56)cc4)c3oc21]

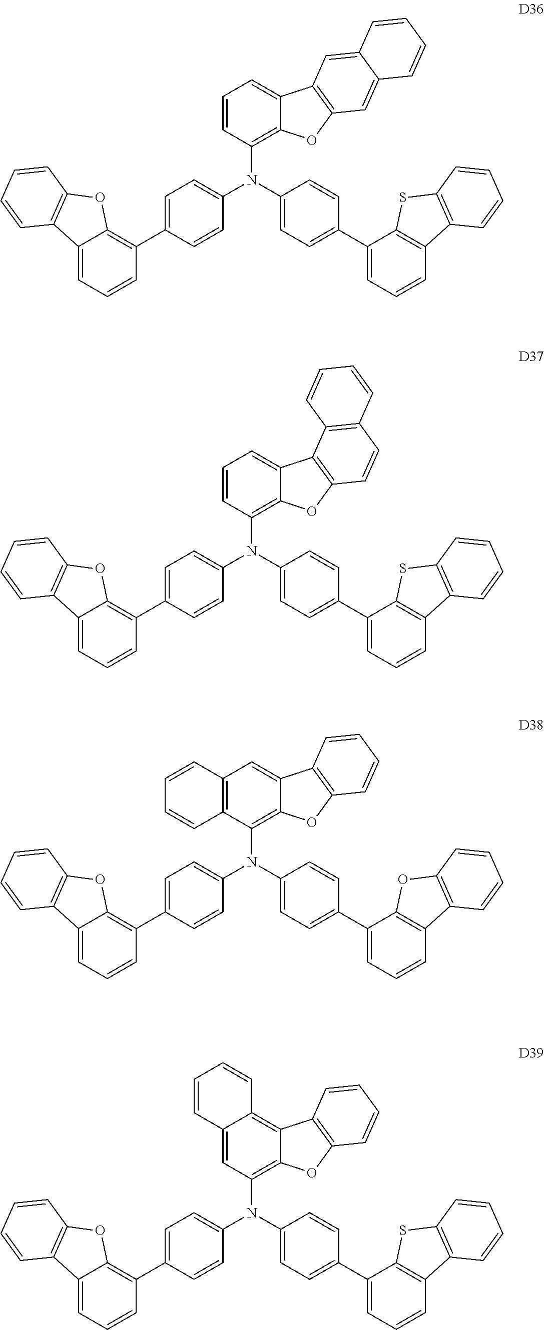 [SMILES: c1ccc2cc3c(cc2c1)oc1c(N(c2ccc(-c4cccc5c4oc4ccccc45)cc2)c2ccc(-c4cccc5c4sc4ccccc45)cc2)cccc13 ; c1ccc2c(c1)ccc1oc3c(N(c4ccc(-c5cccc6c5oc5ccccc56)cc4)c4ccc(-c5cccc6c5sc5ccccc56)cc4)cccc3c12 ; c1ccc2c(N(c3ccc(-c4cccc5c4oc4ccccc45)cc3)c3ccc(-c4cccc5c4oc4ccccc45)cc3)c3oc4ccccc4c3cc2c1 ; c1ccc2c(c1)cc(N(c1ccc(-c3cccc4c3oc3ccccc34)cc1)c1ccc(-c3cccc4c3sc3ccccc34)cc1)c1oc3ccccc3c12]

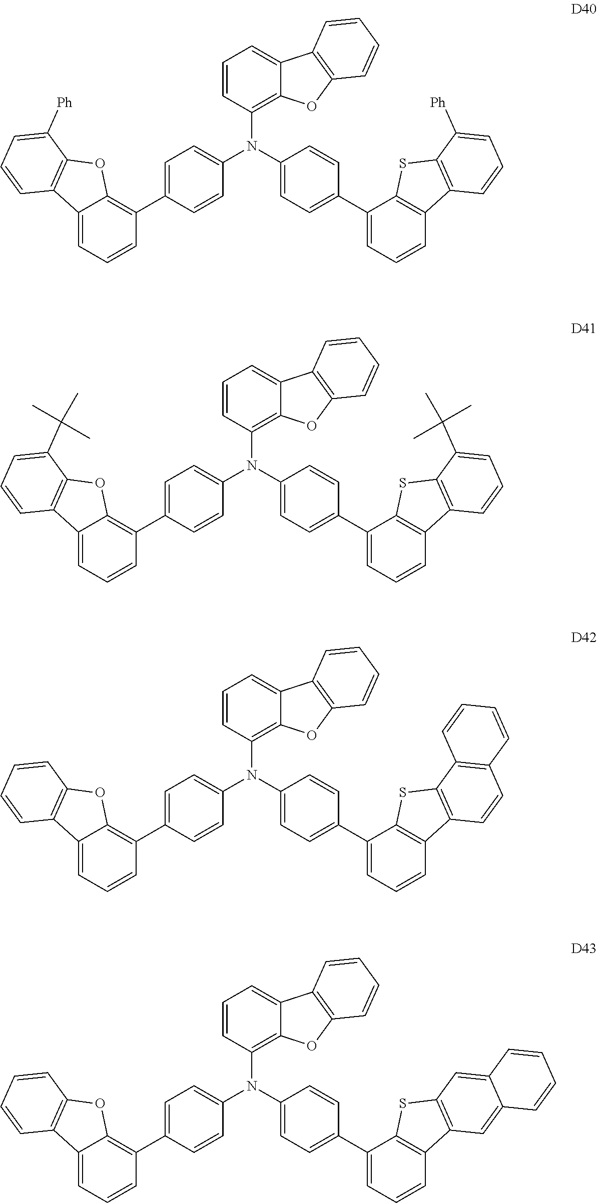 [SMILES: c1ccc(-c2cccc3c2oc2c(-c4ccc(N(c5ccc(-c6cccc7c6sc6c(-c8ccccc8)cccc67)cc5)c5cccc6c5oc5ccccc56)cc4)cccc23)cc1 ; CC(C)(C)c1cccc2c1oc1c(-c3ccc(N(c4ccc(-c5cccc6c5sc5c(C(C)(C)C)cccc56)cc4)c4cccc5c4oc4ccccc45)cc3)cccc12 ; c1ccc2c(c1)ccc1c3cccc(-c4ccc(N(c5ccc(-c6cccc7c6oc6ccccc67)cc5)c5cccc6c5oc5ccccc56)cc4)c3sc21 ; c1ccc2cc3c(cc2c1)sc1c(-c2ccc(N(c4ccc(-c5cccc6c5oc5ccccc56)cc4)c4cccc5c4oc4ccccc45)cc2)cccc13]

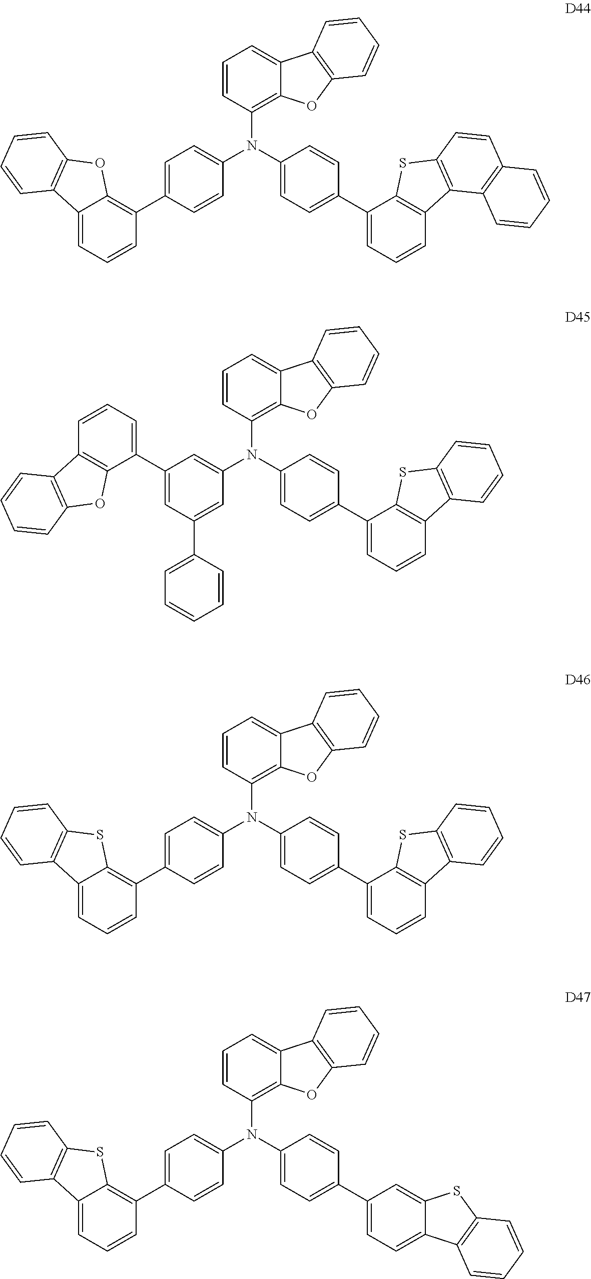 [SMILES: c1ccc2c(c1)ccc1sc3c(-c4ccc(N(c5ccc(-c6cccc7c6oc6ccccc67)cc5)c5cccc6c5oc5ccccc56)cc4)cccc3c12 ; c1ccc(-c2cc(-c3cccc4c3oc3ccccc34)cc(N(c3ccc(-c4cccc5c4sc4ccccc45)cc3)c3cccc4c3oc3ccccc34)c2)cc1 ; c1ccc2c(c1)oc1c(N(c3ccc(-c4cccc5c4sc4ccccc45)cc3)c3ccc(-c4cccc5c4sc4ccccc45)cc3)cccc12 ; c1ccc2c(c1)oc1c(N(c3ccc(-c4ccc5c(c4)sc4ccccc45)cc3)c3ccc(-c4cccc5c4sc4ccccc45)cc3)cccc12]

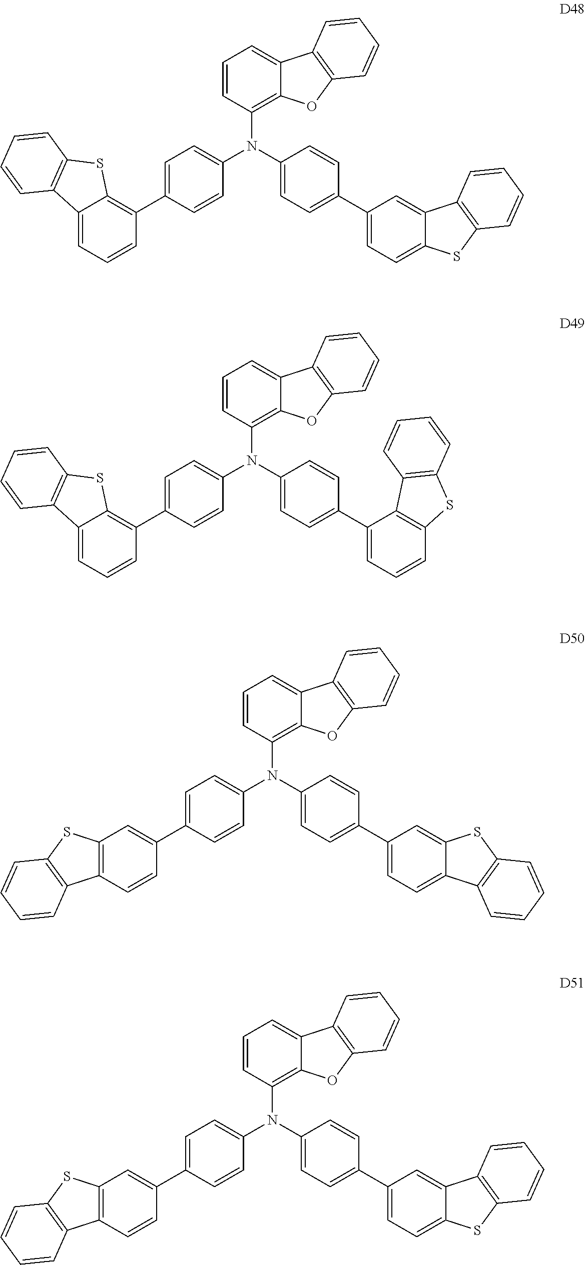 [SMILES: c1ccc2c(c1)oc1c(N(c3ccc(-c4ccc5sc6ccccc6c5c4)cc3)c3ccc(-c4cccc5c4sc4ccccc45)cc3)cccc12 ; c1ccc2c(c1)oc1c(N(c3ccc(-c4cccc5c4sc4ccccc45)cc3)c3ccc(-c4cccc5sc6ccccc6c45)cc3)cccc12 ; c1ccc2c(c1)oc1c(N(c3ccc(-c4ccc5c(c4)sc4ccccc45)cc3)c3ccc(-c4ccc5c(c4)sc4ccccc45)cc3)cccc12 ; c1ccc2c(c1)oc1c(N(c3ccc(-c4ccc5c(c4)sc4ccccc45)cc3)c3ccc(-c4ccc5sc6ccccc6c5c4)cc3)cccc12]

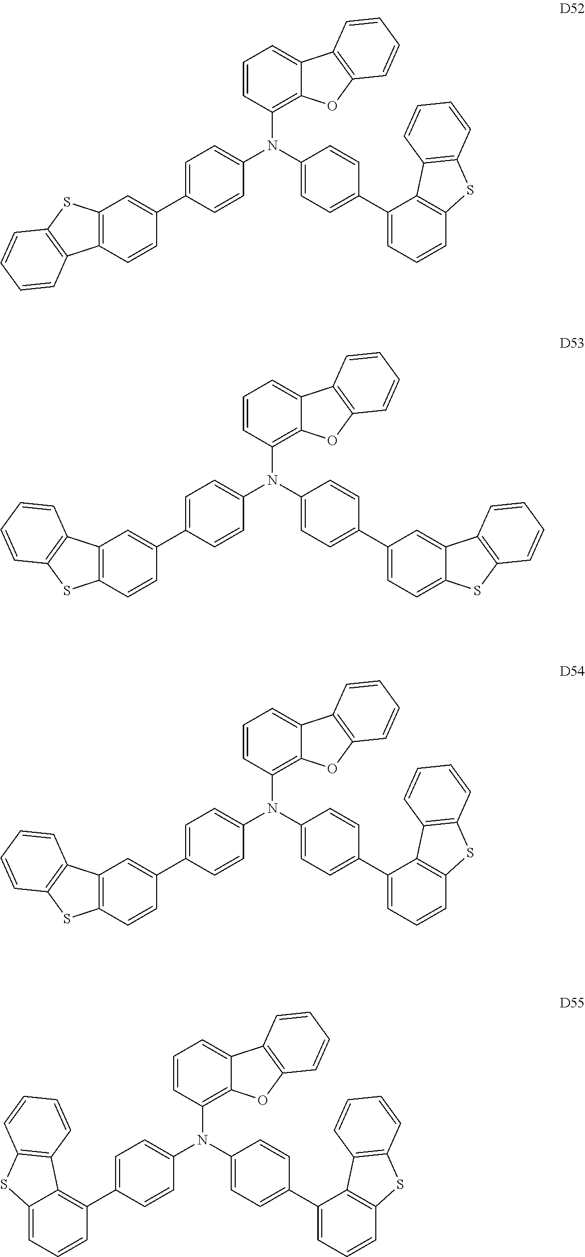 [SMILES: c1ccc2c(c1)oc1c(N(c3ccc(-c4ccc5c(c4)sc4ccccc45)cc3)c3ccc(-c4cccc5sc6ccccc6c45)cc3)cccc12 ; c1ccc2c(c1)oc1c(N(c3ccc(-c4ccc5sc6ccccc6c5c4)cc3)c3ccc(-c4ccc5sc6ccccc6c5c4)cc3)cccc12 ; c1ccc2c(c1)oc1c(N(c3ccc(-c4ccc5sc6ccccc6c5c4)cc3)c3ccc(-c4cccc5sc6ccccc6c45)cc3)cccc12 ; c1ccc2c(c1)oc1c(N(c3ccc(-c4cccc5sc6ccccc6c45)cc3)c3ccc(-c4cccc5sc6ccccc6c45)cc3)cccc12]

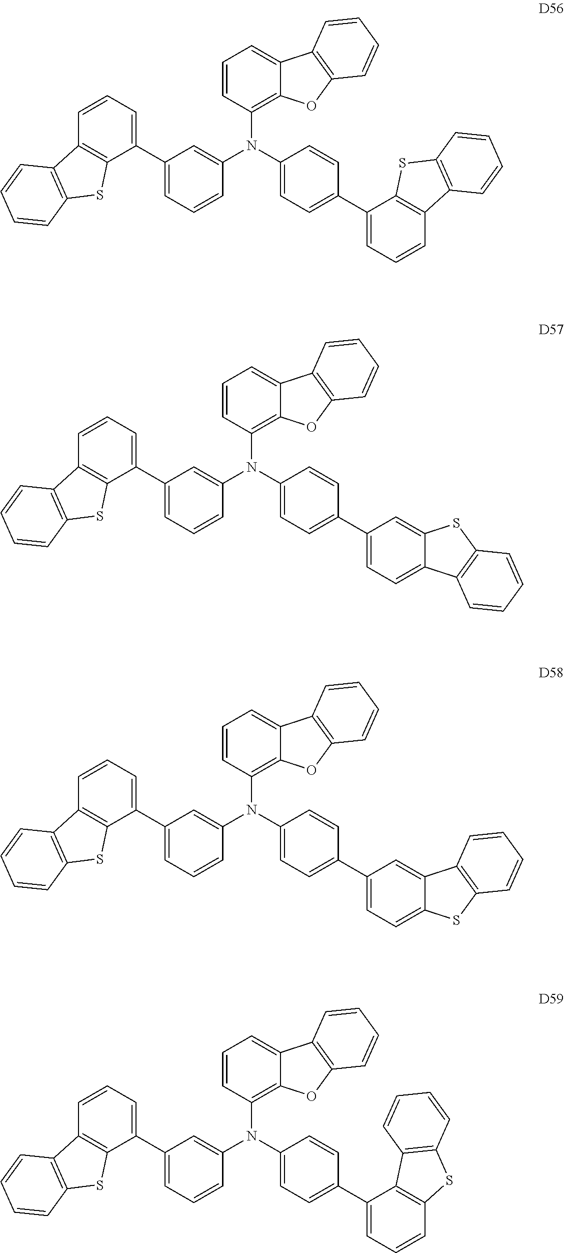 [SMILES: c1cc(-c2cccc3c2sc2ccccc23)cc(N(c2ccc(-c3cccc4c3sc3ccccc34)cc2)c2cccc3c2oc2ccccc23)c1 ; c1cc(-c2cccc3c2sc2ccccc23)cc(N(c2ccc(-c3ccc4c(c3)sc3ccccc34)cc2)c2cccc3c2oc2ccccc23)c1 ; c1cc(-c2cccc3c2sc2ccccc23)cc(N(c2ccc(-c3ccc4sc5ccccc5c4c3)cc2)c2cccc3c2oc2ccccc23)c1 ; c1cc(-c2cccc3c2sc2ccccc23)cc(N(c2ccc(-c3cccc4sc5ccccc5c34)cc2)c2cccc3c2oc2ccccc23)c1]

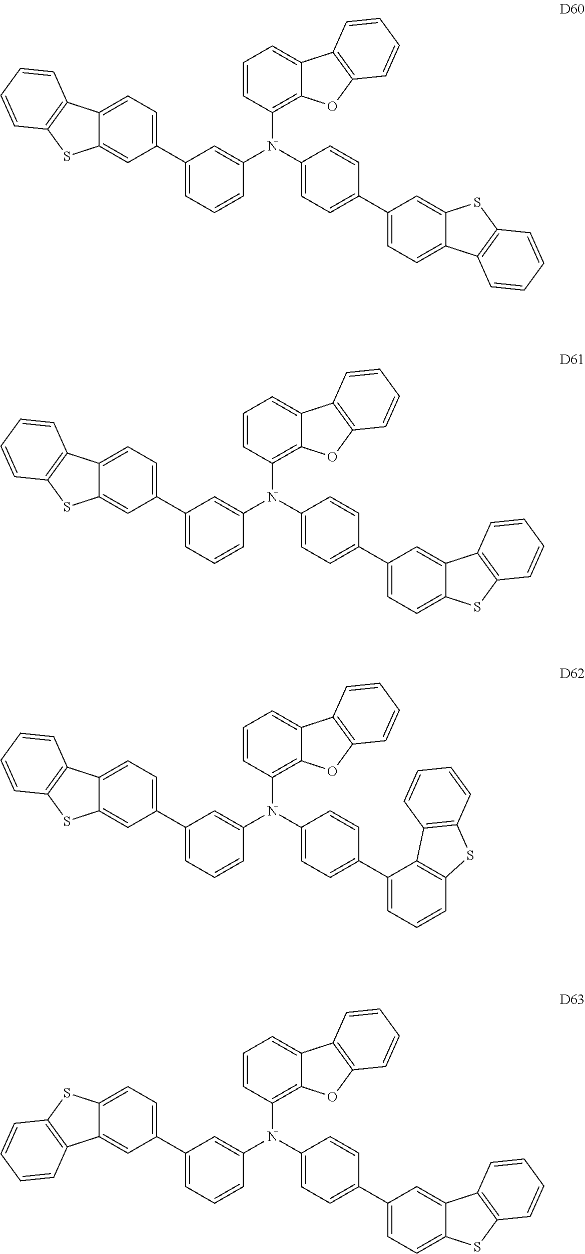 [SMILES: c1cc(-c2ccc3c(c2)sc2ccccc23)cc(N(c2ccc(-c3ccc4c(c3)sc3ccccc34)cc2)c2cccc3c2oc2ccccc23)c1 ; c1cc(-c2ccc3c(c2)sc2ccccc23)cc(N(c2ccc(-c3ccc4sc5ccccc5c4c3)cc2)c2cccc3c2oc2ccccc23)c1 ; c1cc(-c2ccc3c(c2)sc2ccccc23)cc(N(c2ccc(-c3cccc4sc5ccccc5c34)cc2)c2cccc3c2oc2ccccc23)c1 ; c1cc(-c2ccc3sc4ccccc4c3c2)cc(N(c2ccc(-c3ccc4sc5ccccc5c4c3)cc2)c2cccc3c2oc2ccccc23)c1]

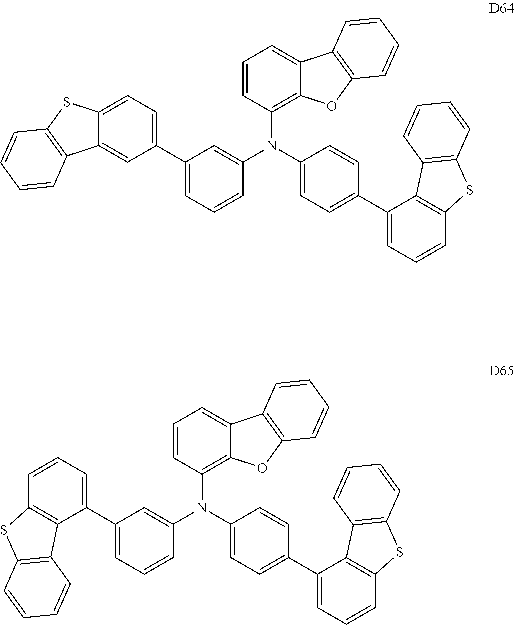 [SMILES: c1cc(-c2ccc3sc4ccccc4c3c2)cc(N(c2ccc(-c3cccc4sc5ccccc5c34)cc2)c2cccc3c2oc2ccccc23)c1 ; c1cc(-c2cccc3sc4ccccc4c23)cc(N(c2ccc(-c3cccc4sc5ccccc5c34)cc2)c2cccc3c2oc2ccccc23)c1]

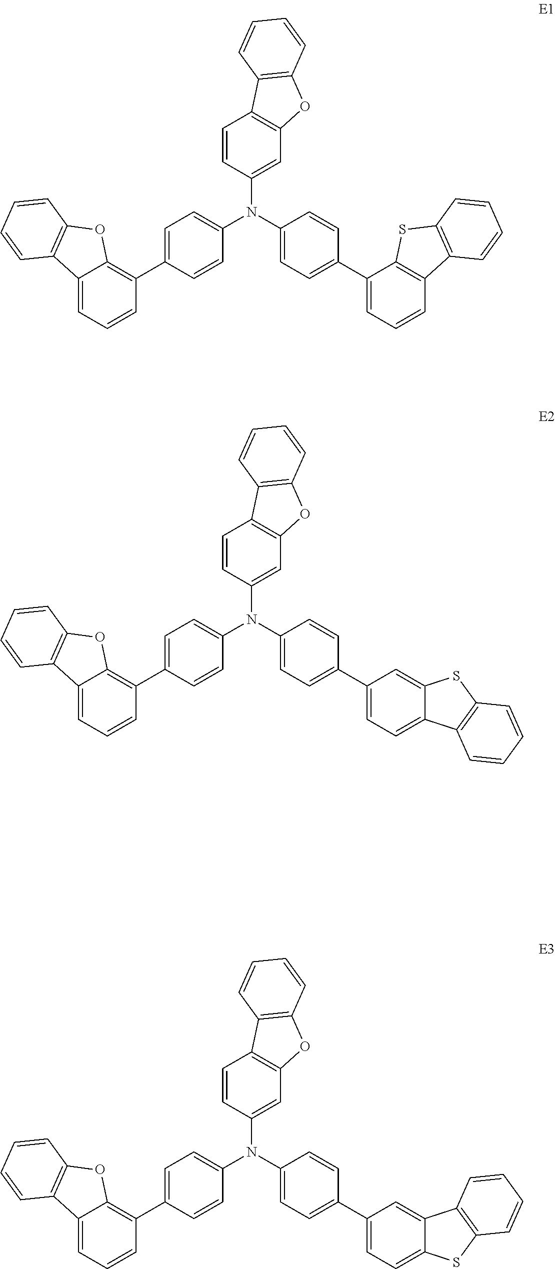 [SMILES: c1ccc2c(c1)oc1cc(N(c3ccc(-c4cccc5c4oc4ccccc45)cc3)c3ccc(-c4cccc5c4sc4ccccc45)cc3)ccc12 ; c1ccc2c(c1)oc1cc(N(c3ccc(-c4ccc5c(c4)sc4ccccc45)cc3)c3ccc(-c4cccc5c4oc4ccccc45)cc3)ccc12 ; c1ccc2c(c1)oc1cc(N(c3ccc(-c4ccc5sc6ccccc6c5c4)cc3)c3ccc(-c4cccc5c4oc4ccccc45)cc3)ccc12]

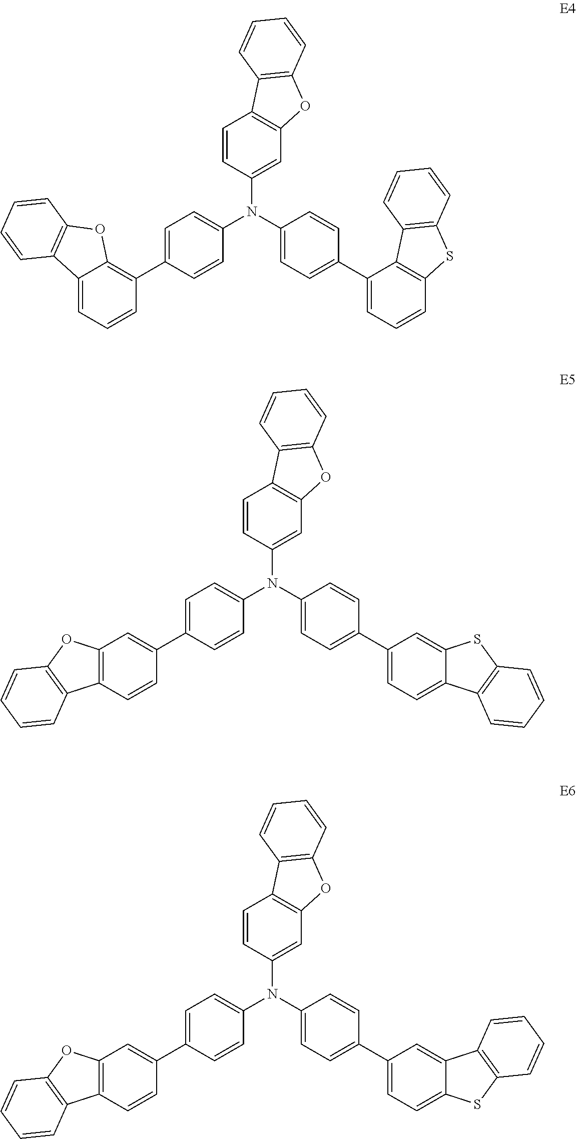 [SMILES: c1ccc2c(c1)oc1cc(N(c3ccc(-c4cccc5c4oc4ccccc45)cc3)c3ccc(-c4cccc5sc6ccccc6c45)cc3)ccc12 ; c1ccc2c(c1)oc1cc(-c3ccc(N(c4ccc(-c5ccc6c(c5)sc5ccccc56)cc4)c4ccc5c(c4)oc4ccccc45)cc3)ccc12 ; c1ccc2c(c1)oc1cc(-c3ccc(N(c4ccc(-c5ccc6sc7ccccc7c6c5)cc4)c4ccc5c(c4)oc4ccccc45)cc3)ccc12]

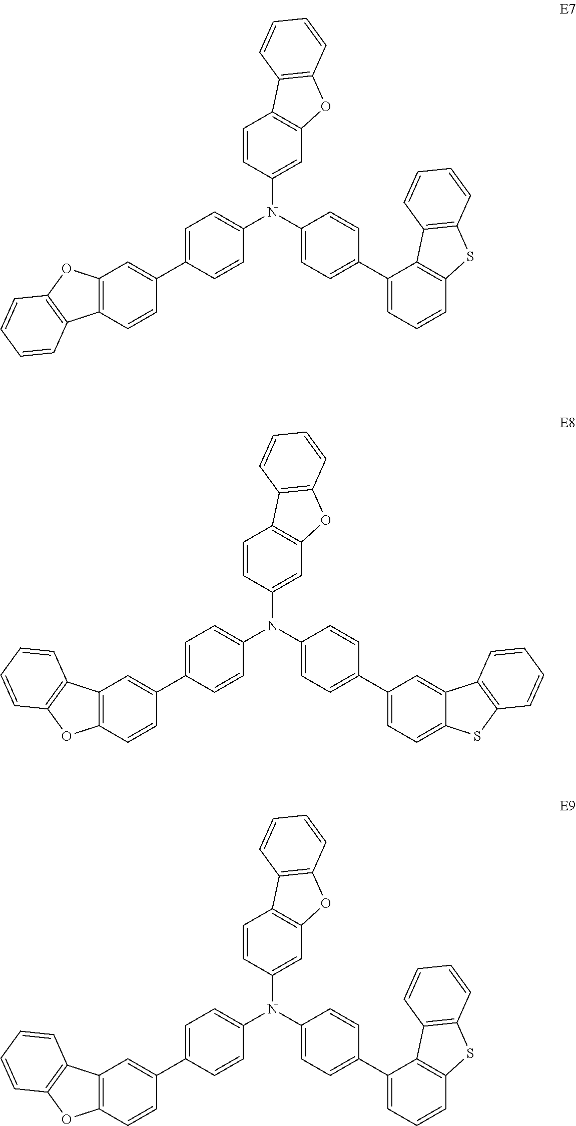 [SMILES: c1ccc2c(c1)oc1cc(-c3ccc(N(c4ccc(-c5cccc6sc7ccccc7c56)cc4)c4ccc5c(c4)oc4ccccc45)cc3)ccc12 ; c1ccc2c(c1)oc1cc(N(c3ccc(-c4ccc5oc6ccccc6c5c4)cc3)c3ccc(-c4ccc5sc6ccccc6c5c4)cc3)ccc12 ; c1ccc2c(c1)oc1cc(N(c3ccc(-c4ccc5oc6ccccc6c5c4)cc3)c3ccc(-c4cccc5sc6ccccc6c45)cc3)ccc12]

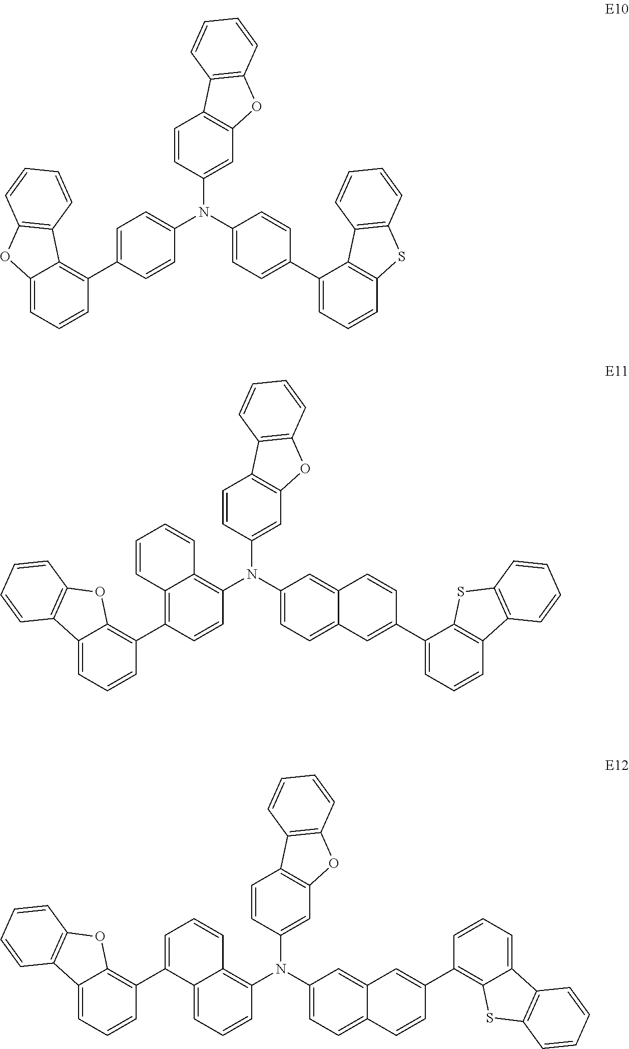 [SMILES: c1ccc2c(c1)oc1cc(N(c3ccc(-c4cccc5oc6ccccc6c45)cc3)c3ccc(-c4cccc5sc6ccccc6c45)cc3)ccc12 ; c1ccc2c(c1)oc1cc(N(c3ccc4cc(-c5cccc6c5sc5ccccc56)ccc4c3)c3ccc(-c4cccc5c4oc4ccccc45)c4ccccc34)ccc12 ; c1ccc2c(c1)oc1cc(N(c3ccc4ccc(-c5cccc6c5sc5ccccc56)cc4c3)c3cccc4c(-c5cccc6c5oc5ccccc56)cccc34)ccc12]

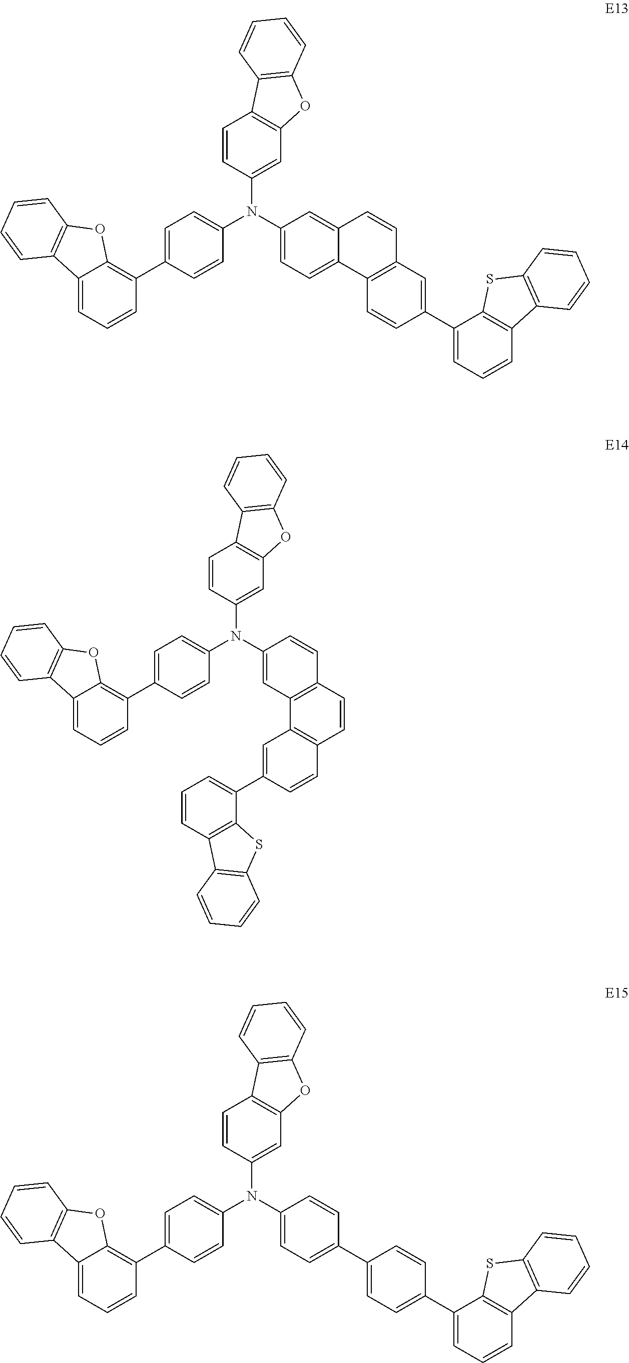 [SMILES: c1ccc2c(c1)oc1cc(N(c3ccc(-c4cccc5c4oc4ccccc45)cc3)c3ccc4c(ccc5cc(-c6cccc7c6sc6ccccc67)ccc54)c3)ccc12 ; c1ccc2c(c1)oc1cc(N(c3ccc(-c4cccc5c4oc4ccccc45)cc3)c3ccc4ccc5ccc(-c6cccc7c6sc6ccccc67)cc5c4c3)ccc12 ; c1ccc2c(c1)oc1cc(N(c3ccc(-c4ccc(-c5cccc6c5sc5ccccc56)cc4)cc3)c3ccc(-c4cccc5c4oc4ccccc45)cc3)ccc12]

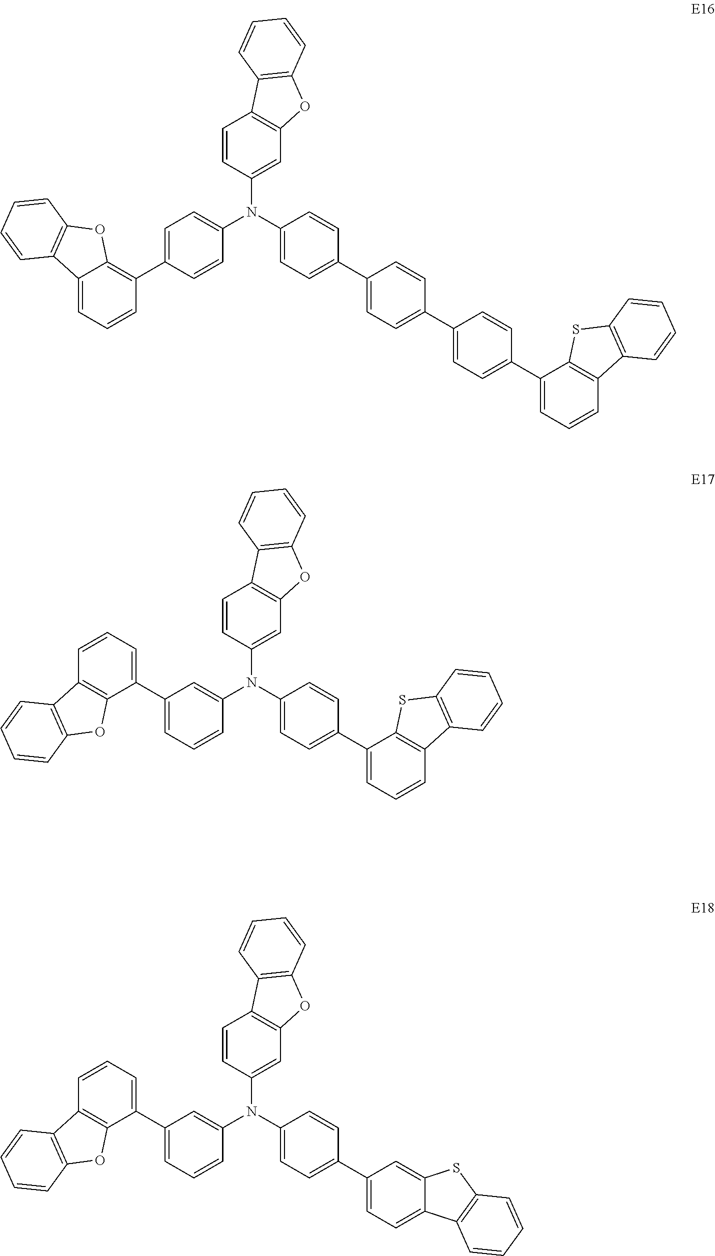 [SMILES: c1ccc2c(c1)oc1cc(N(c3ccc(-c4ccc(-c5ccc(-c6cccc7c6sc6ccccc67)cc5)cc4)cc3)c3ccc(-c4cccc5c4oc4ccccc45)cc3)ccc12 ; c1cc(-c2cccc3c2oc2ccccc23)cc(N(c2ccc(-c3cccc4c3sc3ccccc34)cc2)c2ccc3c(c2)oc2ccccc23)c1 ; c1cc(-c2cccc3c2oc2ccccc23)cc(N(c2ccc(-c3ccc4c(c3)sc3ccccc34)cc2)c2ccc3c(c2)oc2ccccc23)c1]

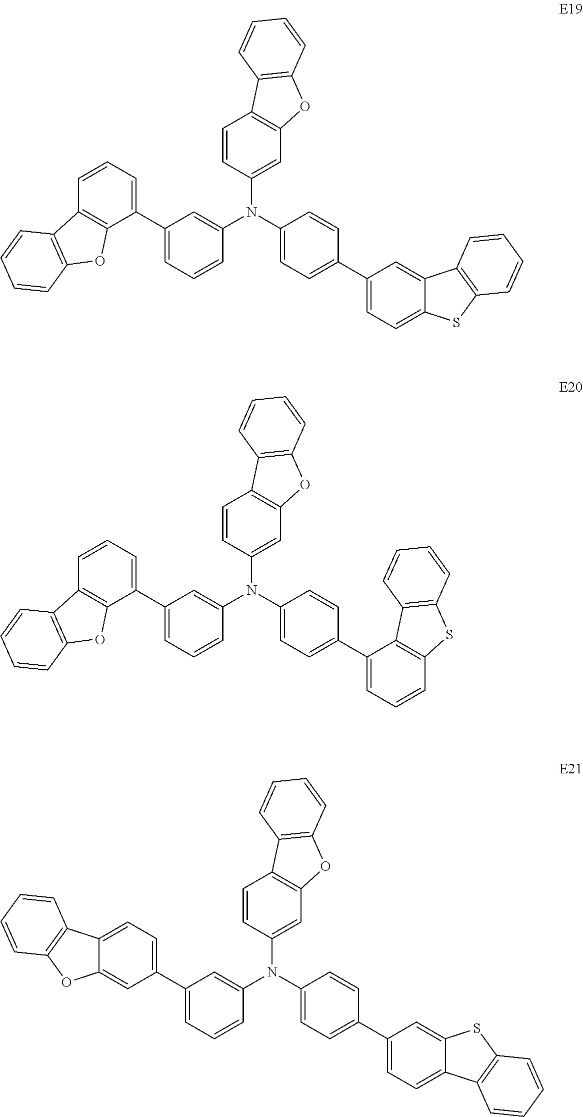 [SMILES: c1cc(-c2cccc3c2oc2ccccc23)cc(N(c2ccc(-c3ccc4sc5ccccc5c4c3)cc2)c2ccc3c(c2)oc2ccccc23)c1 ; c1cc(-c2cccc3c2oc2ccccc23)cc(N(c2ccc(-c3cccc4sc5ccccc5c34)cc2)c2ccc3c(c2)oc2ccccc23)c1 ; c1cc(-c2ccc3c(c2)oc2ccccc23)cc(N(c2ccc(-c3ccc4c(c3)sc3ccccc34)cc2)c2ccc3c(c2)oc2ccccc23)c1]

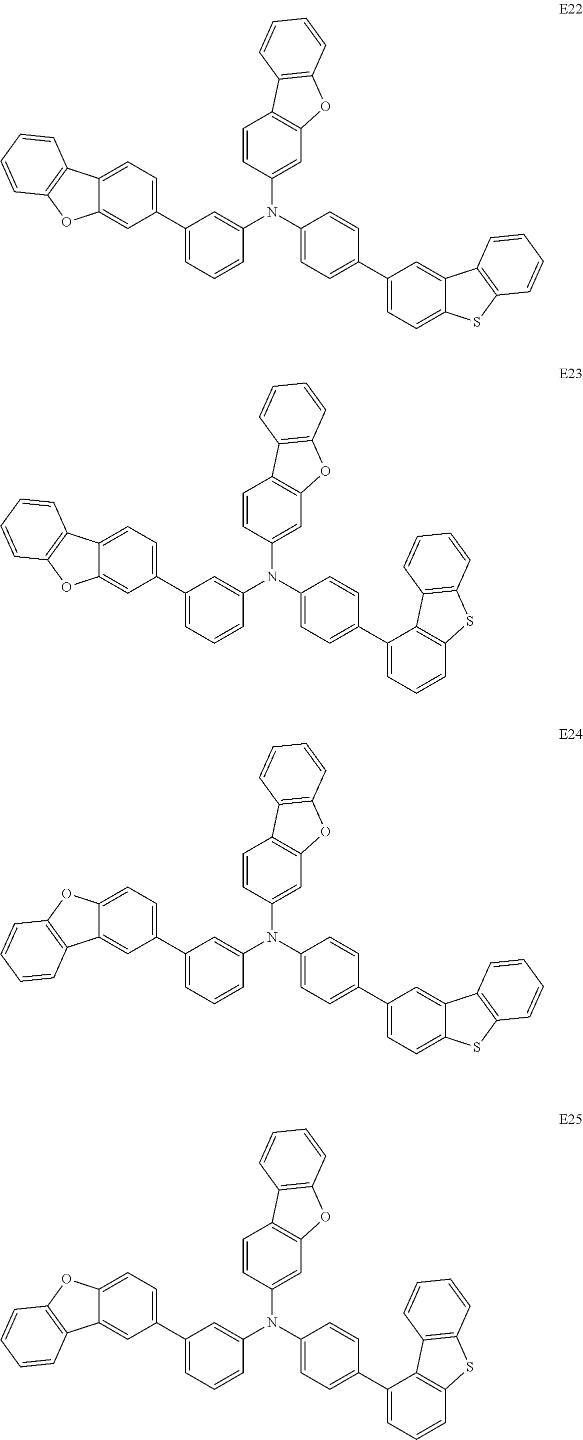 [SMILES: c1cc(-c2ccc3c(c2)oc2ccccc23)cc(N(c2ccc(-c3ccc4sc5ccccc5c4c3)cc2)c2ccc3c(c2)oc2ccccc23)c1 ; c1cc(-c2ccc3c(c2)oc2ccccc23)cc(N(c2ccc(-c3cccc4sc5ccccc5c34)cc2)c2ccc3c(c2)oc2ccccc23)c1 ; c1cc(-c2ccc3oc4ccccc4c3c2)cc(N(c2ccc(-c3ccc4sc5ccccc5c4c3)cc2)c2ccc3c(c2)oc2ccccc23)c1 ; c1cc(-c2ccc3oc4ccccc4c3c2)cc(N(c2ccc(-c3cccc4sc5ccccc5c34)cc2)c2ccc3c(c2)oc2ccccc23)c1]

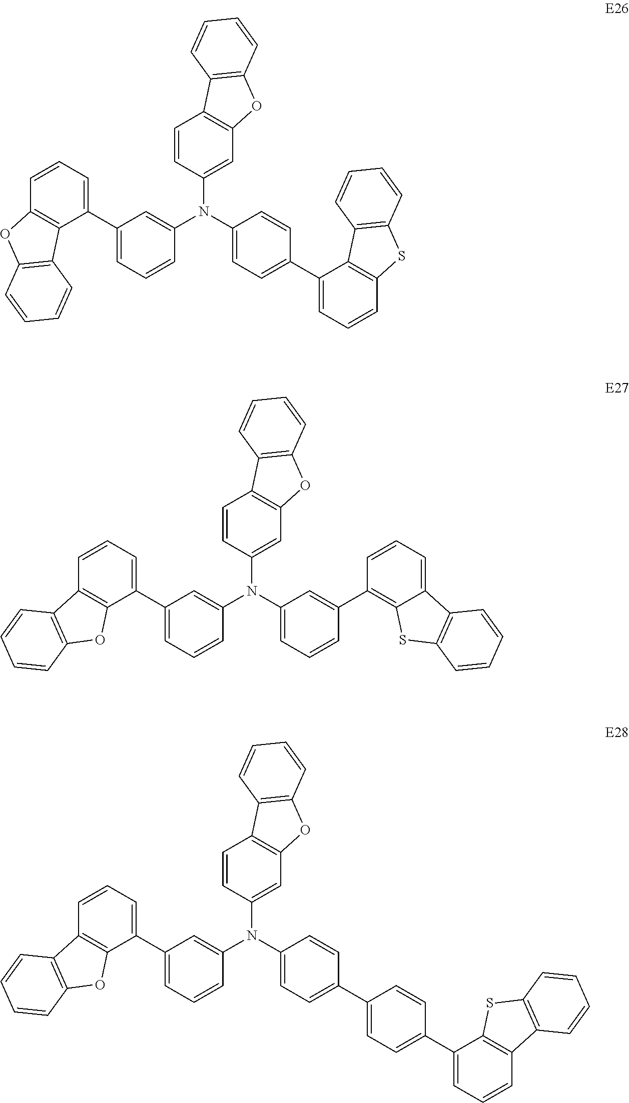 [SMILES: c1cc(-c2cccc3oc4ccccc4c23)cc(N(c2ccc(-c3cccc4sc5ccccc5c34)cc2)c2ccc3c(c2)oc2ccccc23)c1 ; c1cc(-c2cccc3c2oc2ccccc23)cc(N(c2cccc(-c3cccc4c3sc3ccccc34)c2)c2ccc3c(c2)oc2ccccc23)c1 ; c1cc(-c2cccc3c2oc2ccccc23)cc(N(c2ccc(-c3ccc(-c4cccc5c4sc4ccccc45)cc3)cc2)c2ccc3c(c2)oc2ccccc23)c1]

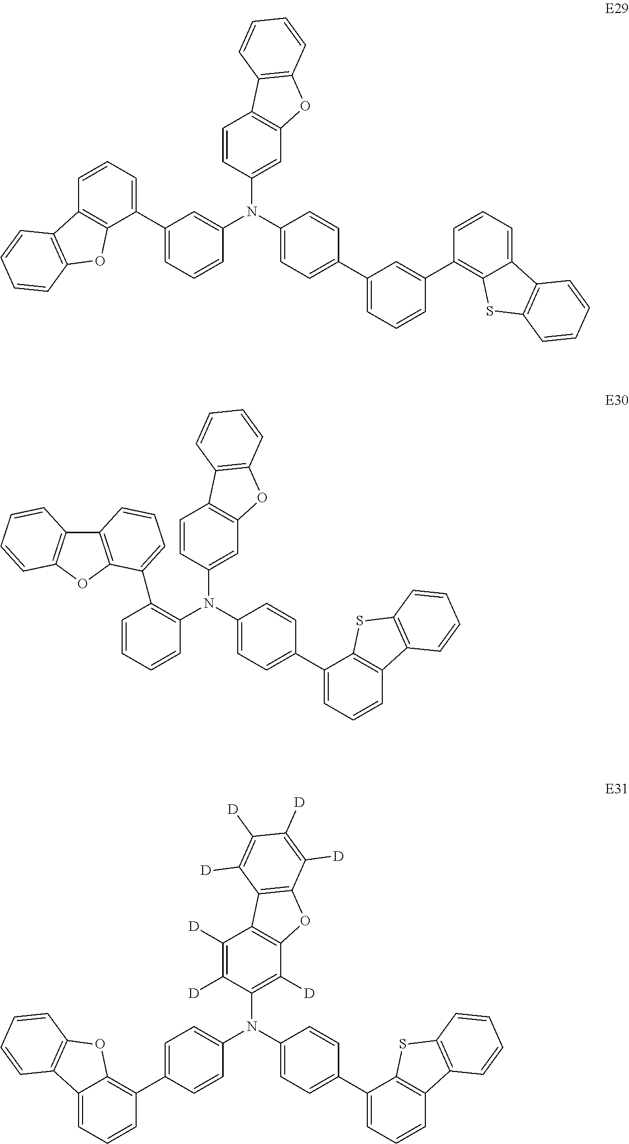 [SMILES: c1cc(-c2ccc(N(c3cccc(-c4cccc5c4oc4ccccc45)c3)c3ccc4c(c3)oc3ccccc34)cc2)cc(-c2cccc3c2sc2ccccc23)c1 ; c1ccc(N(c2ccc(-c3cccc4c3sc3ccccc34)cc2)c2ccc3c(c2)oc2ccccc23)c(-c2cccc3c2oc2ccccc23)c1 ; [2H]c1c([2H])c([2H])c2c(oc3c([2H])c(N(c4ccc(-c5cccc6c5oc5ccccc56)cc4)c4ccc(-c5cccc6c5sc5ccccc56)cc4)c([2H])c([2H])c32)c1[2H]]

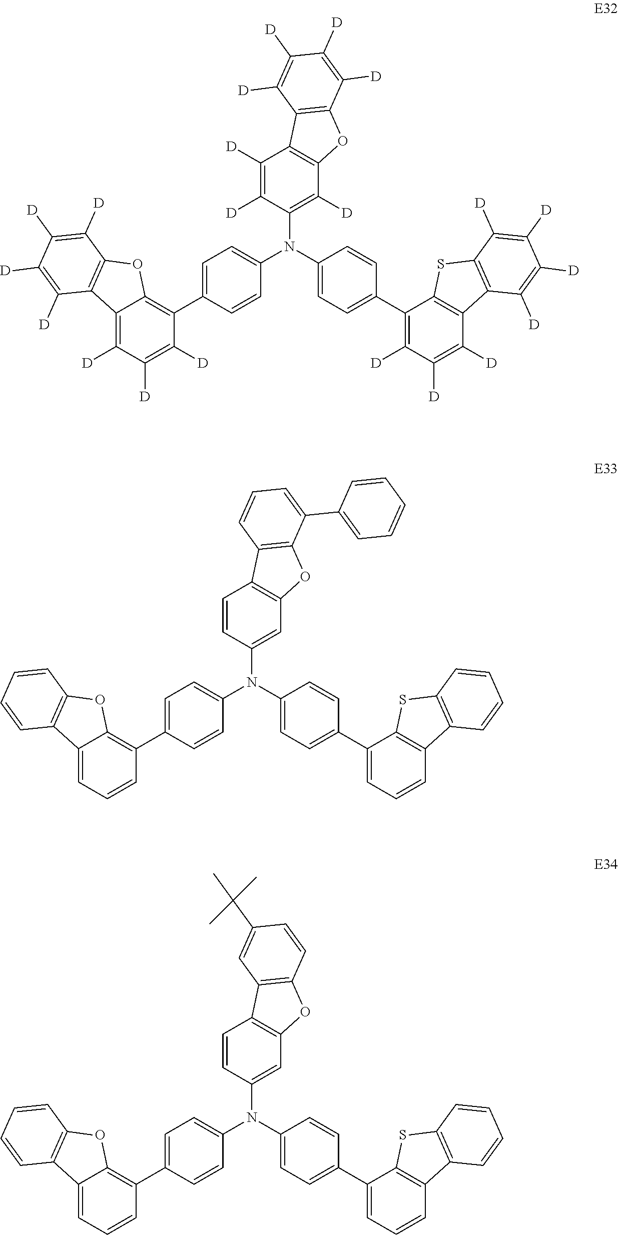 [SMILES: [2H]c1c([2H])c([2H])c2c(oc3c([2H])c(N(c4ccc(-c5c([2H])c([2H])c([2H])c6c5oc5c([2H])c([2H])c([2H])c([2H])c56)cc4)c4ccc(-c5c([2H])c([2H])c([2H])c6c5sc5c([2H])c([2H])c([2H])c([2H])c56)cc4)c([2H])c([2H])c32)c1[2H] ; c1ccc(-c2cccc3c2oc2cc(N(c4ccc(-c5cccc6c5oc5ccccc56)cc4)c4ccc(-c5cccc6c5sc5ccccc56)cc4)ccc23)cc1 ; CC(C)(C)c1ccc2oc3cc(N(c4ccc(-c5cccc6c5oc5ccccc56)cc4)c4ccc(-c5cccc6c5sc5ccccc56)cc4)ccc3c2c1]

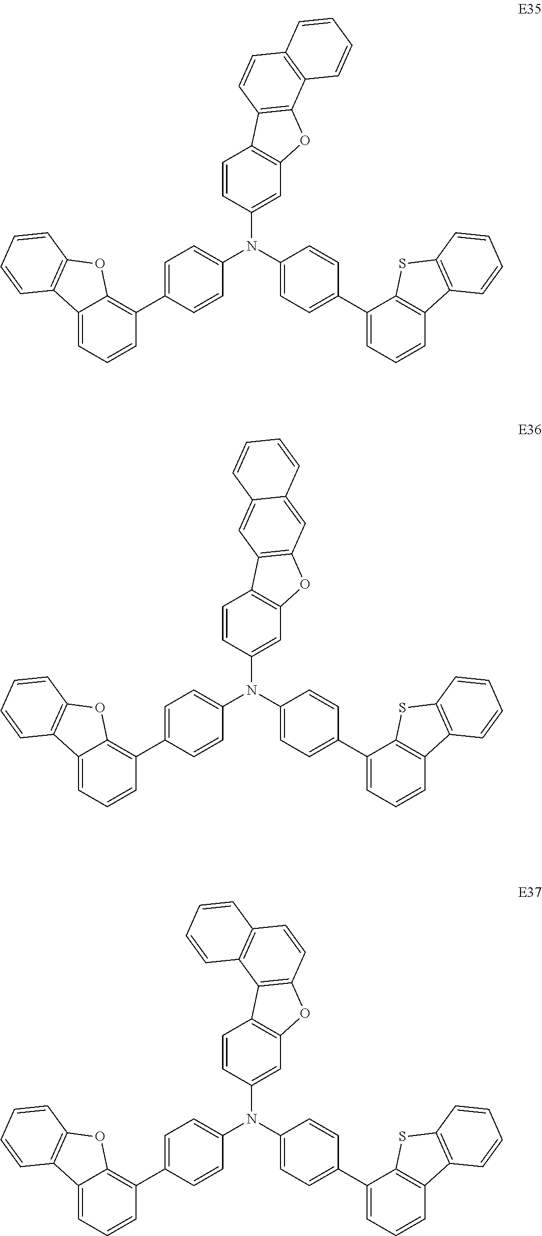 [SMILES: c1ccc2c(c1)ccc1c3ccc(N(c4ccc(-c5cccc6c5oc5ccccc56)cc4)c4ccc(-c5cccc6c5sc5ccccc56)cc4)cc3oc21 ; c1ccc2cc3c(cc2c1)oc1cc(N(c2ccc(-c4cccc5c4oc4ccccc45)cc2)c2ccc(-c4cccc5c4sc4ccccc45)cc2)ccc13 ; c1ccc2c(c1)ccc1oc3cc(N(c4ccc(-c5cccc6c5oc5ccccc56)cc4)c4ccc(-c5cccc6c5sc5ccccc56)cc4)ccc3c12]

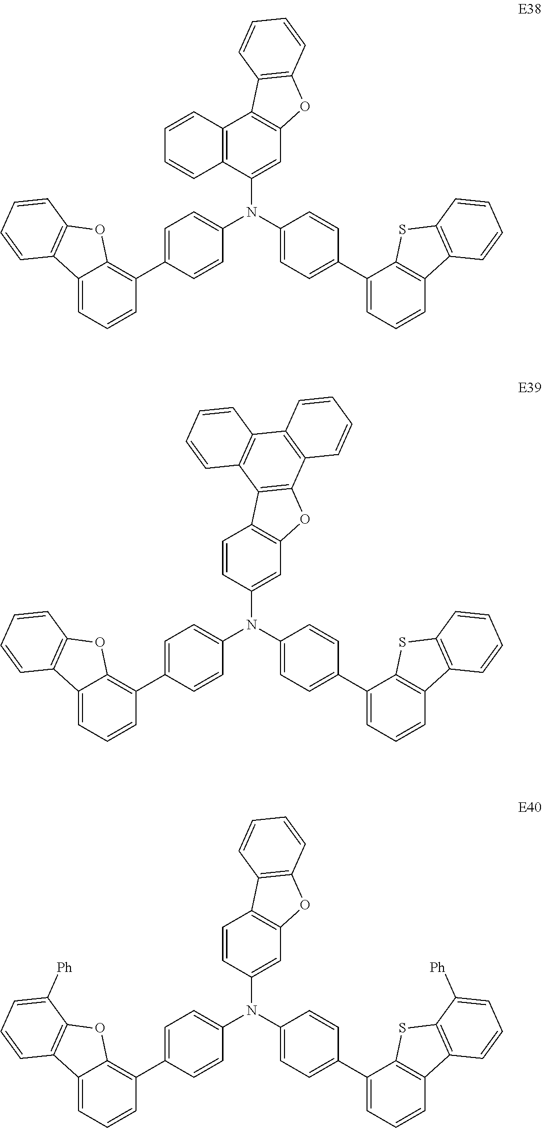 [SMILES: c1ccc2c(c1)oc1c(-c3ccc(N(c4ccc(-c5cccc6c5sc5ccccc56)cc4)c4cc5oc6ccccc6c5c5ccccc45)cc3)cccc12 ; c1ccc2c(c1)oc1c(-c3ccc(N(c4ccc(-c5cccc6c5sc5ccccc56)cc4)c4ccc5c(c4)oc4c6ccccc6c6ccccc6c54)cc3)cccc12 ; c1ccc(-c2cccc3c2oc2c(-c4ccc(N(c5ccc(-c6cccc7c6sc6c(-c8ccccc8)cccc67)cc5)c5ccc6c(c5)oc5ccccc56)cc4)cccc23)cc1]

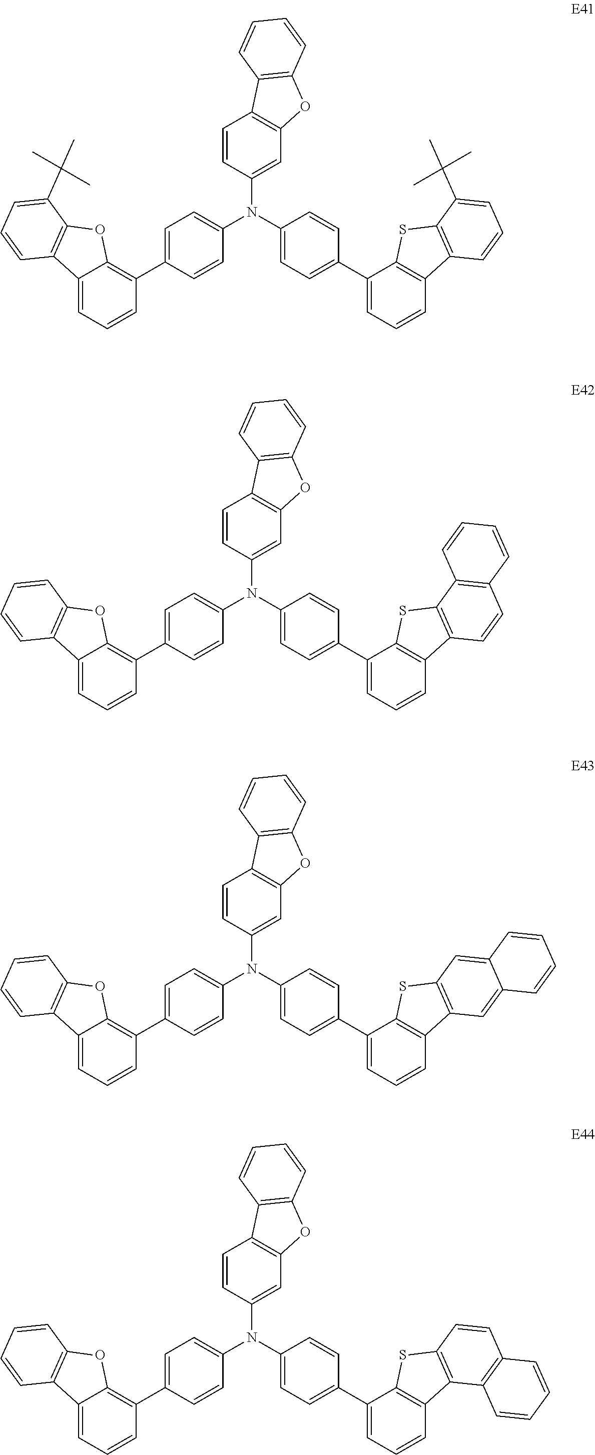 [SMILES: CC(C)(C)c1cccc2c1oc1c(-c3ccc(N(c4ccc(-c5cccc6c5sc5c(C(C)(C)C)cccc56)cc4)c4ccc5c(c4)oc4ccccc45)cc3)cccc12 ; c1ccc2c(c1)ccc1c3cccc(-c4ccc(N(c5ccc(-c6cccc7c6oc6ccccc67)cc5)c5ccc6c(c5)oc5ccccc56)cc4)c3sc21 ; c1ccc2cc3c(cc2c1)sc1c(-c2ccc(N(c4ccc(-c5cccc6c5oc5ccccc56)cc4)c4ccc5c(c4)oc4ccccc45)cc2)cccc13 ; c1ccc2c(c1)ccc1sc3c(-c4ccc(N(c5ccc(-c6cccc7c6oc6ccccc67)cc5)c5ccc6c(c5)oc5ccccc56)cc4)cccc3c12]

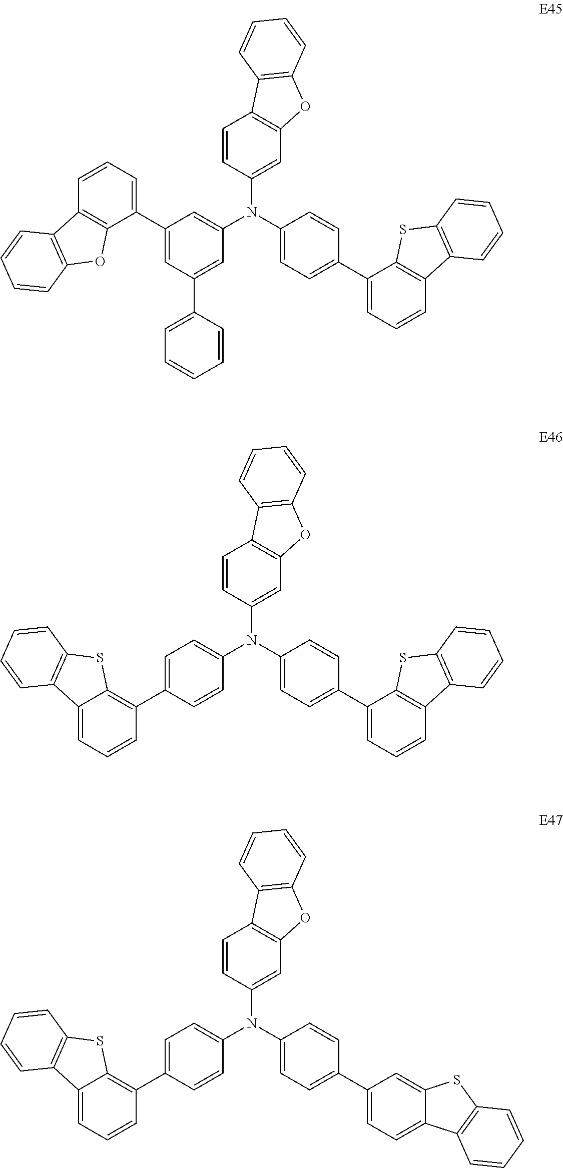 [SMILES: c1ccc(-c2cc(-c3cccc4c3oc3ccccc34)cc(N(c3ccc(-c4cccc5c4sc4ccccc45)cc3)c3ccc4c(c3)oc3ccccc34)c2)cc1 ; c1ccc2c(c1)oc1cc(N(c3ccc(-c4cccc5c4sc4ccccc45)cc3)c3ccc(-c4cccc5c4sc4ccccc45)cc3)ccc12 ; c1ccc2c(c1)oc1cc(N(c3ccc(-c4ccc5c(c4)sc4ccccc45)cc3)c3ccc(-c4cccc5c4sc4ccccc45)cc3)ccc12]

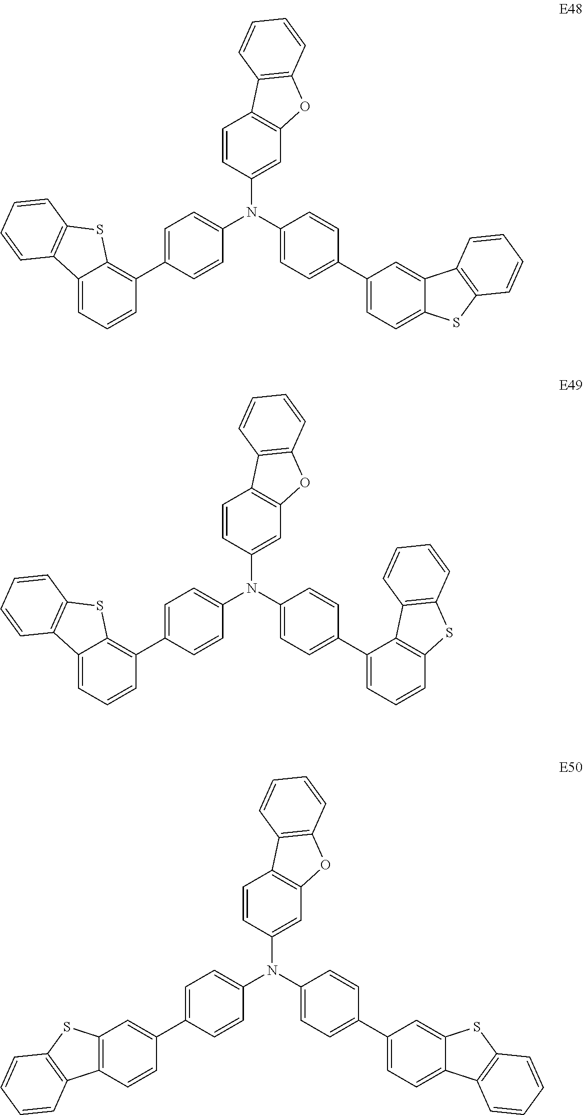 [SMILES: c1ccc2c(c1)oc1cc(N(c3ccc(-c4ccc5sc6ccccc6c5c4)cc3)c3ccc(-c4cccc5c4sc4ccccc45)cc3)ccc12 ; c1ccc2c(c1)oc1cc(N(c3ccc(-c4cccc5c4sc4ccccc45)cc3)c3ccc(-c4cccc5sc6ccccc6c45)cc3)ccc12 ; c1ccc2c(c1)oc1cc(N(c3ccc(-c4ccc5c(c4)sc4ccccc45)cc3)c3ccc(-c4ccc5c(c4)sc4ccccc45)cc3)ccc12]

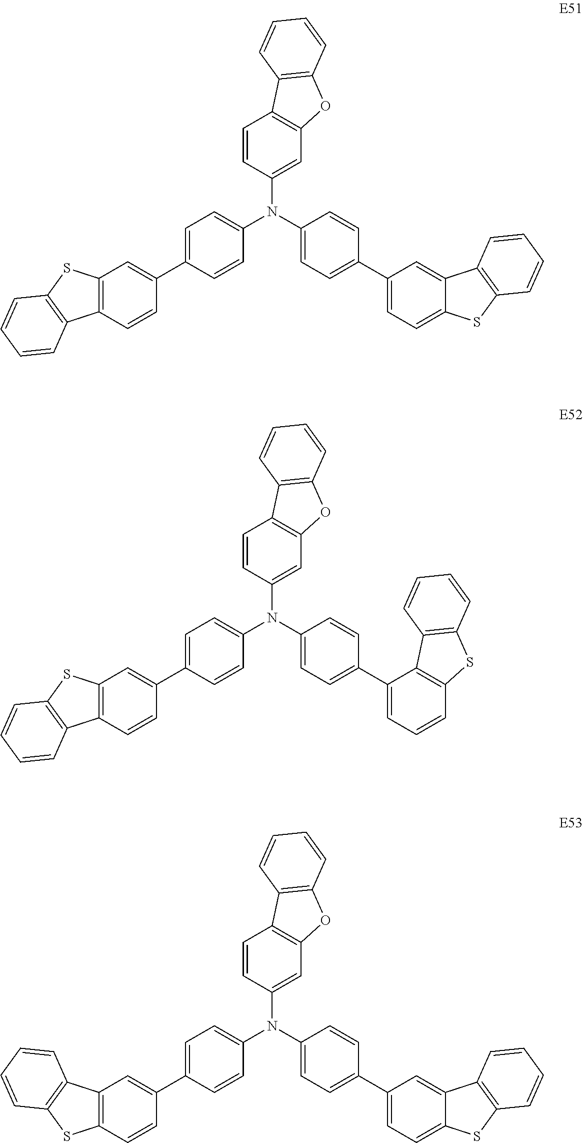 [SMILES: c1ccc2c(c1)oc1cc(N(c3ccc(-c4ccc5c(c4)sc4ccccc45)cc3)c3ccc(-c4ccc5sc6ccccc6c5c4)cc3)ccc12 ; c1ccc2c(c1)oc1cc(N(c3ccc(-c4ccc5c(c4)sc4ccccc45)cc3)c3ccc(-c4cccc5sc6ccccc6c45)cc3)ccc12 ; c1ccc2c(c1)oc1cc(N(c3ccc(-c4ccc5sc6ccccc6c5c4)cc3)c3ccc(-c4ccc5sc6ccccc6c5c4)cc3)ccc12]

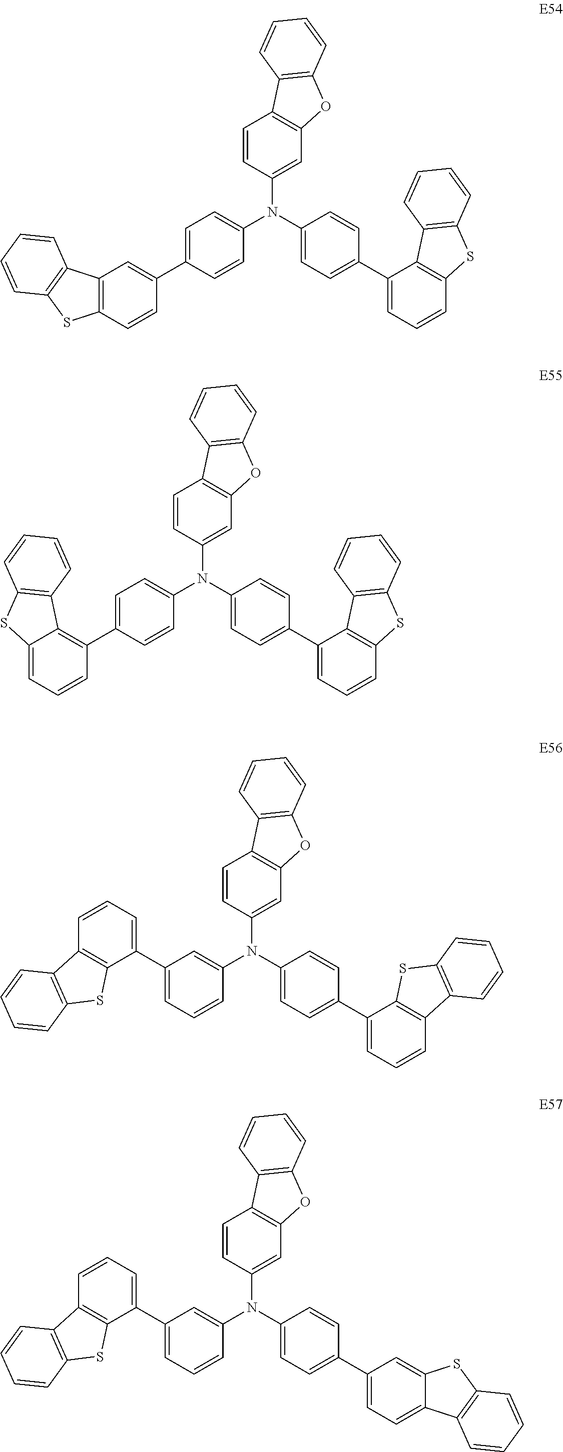 [SMILES: c1ccc2c(c1)oc1cc(N(c3ccc(-c4ccc5sc6ccccc6c5c4)cc3)c3ccc(-c4cccc5sc6ccccc6c45)cc3)ccc12 ; c1ccc2c(c1)oc1cc(N(c3ccc(-c4cccc5sc6ccccc6c45)cc3)c3ccc(-c4cccc5sc6ccccc6c45)cc3)ccc12 ; c1cc(-c2cccc3c2sc2ccccc23)cc(N(c2ccc(-c3cccc4c3sc3ccccc34)cc2)c2ccc3c(c2)oc2ccccc23)c1 ; c1cc(-c2cccc3c2sc2ccccc23)cc(N(c2ccc(-c3ccc4c(c3)sc3ccccc34)cc2)c2ccc3c(c2)oc2ccccc23)c1]

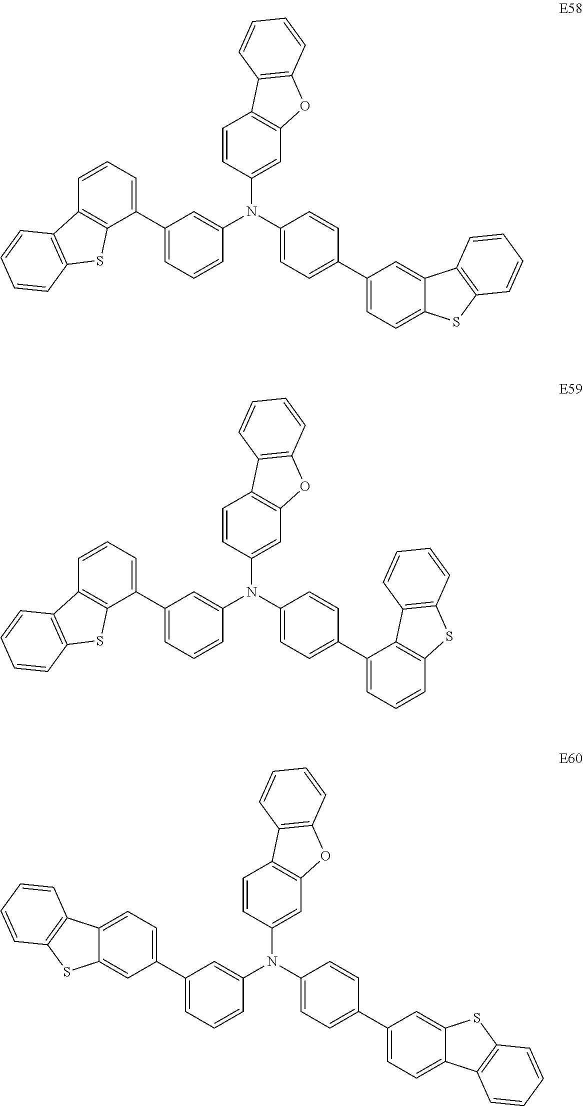 [SMILES: c1cc(-c2cccc3c2sc2ccccc23)cc(N(c2ccc(-c3ccc4sc5ccccc5c4c3)cc2)c2ccc3c(c2)oc2ccccc23)c1 ; c1cc(-c2cccc3c2sc2ccccc23)cc(N(c2ccc(-c3cccc4sc5ccccc5c34)cc2)c2ccc3c(c2)oc2ccccc23)c1 ; c1cc(-c2ccc3c(c2)sc2ccccc23)cc(N(c2ccc(-c3ccc4c(c3)sc3ccccc34)cc2)c2ccc3c(c2)oc2ccccc23)c1]

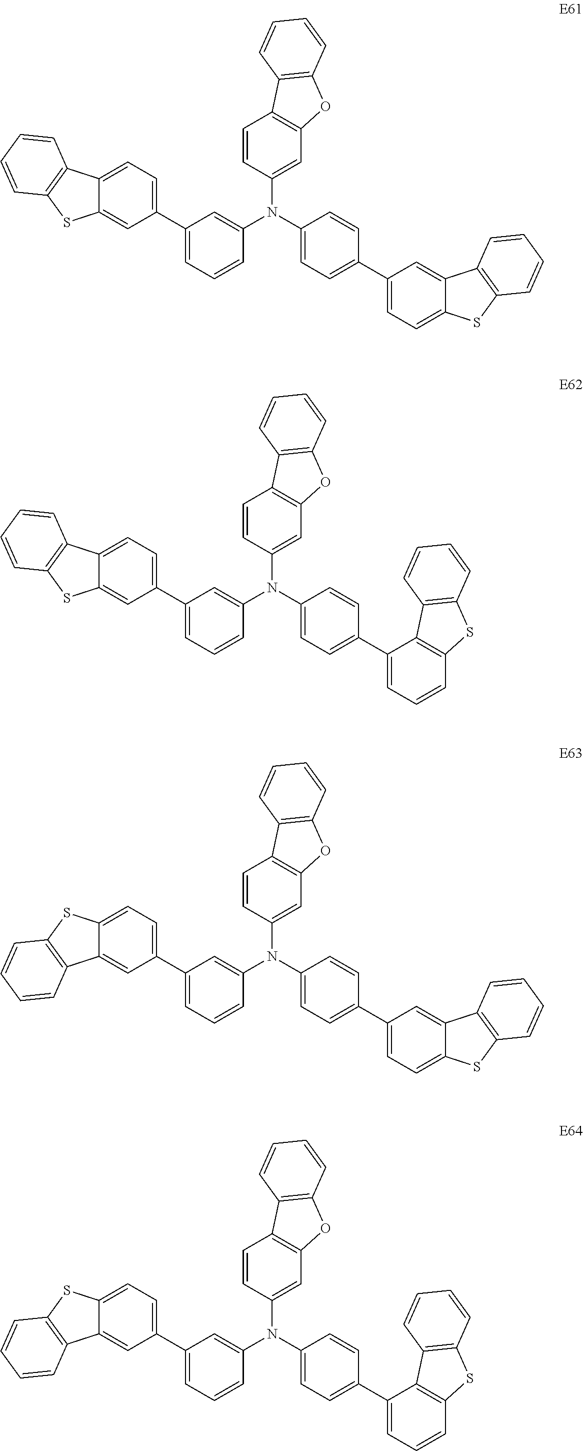 [SMILES: c1cc(-c2ccc3c(c2)sc2ccccc23)cc(N(c2ccc(-c3ccc4sc5ccccc5c4c3)cc2)c2ccc3c(c2)oc2ccccc23)c1 ; c1cc(-c2ccc3c(c2)sc2ccccc23)cc(N(c2ccc(-c3cccc4sc5ccccc5c34)cc2)c2ccc3c(c2)oc2ccccc23)c1 ; c1cc(-c2ccc3sc4ccccc4c3c2)cc(N(c2ccc(-c3ccc4sc5ccccc5c4c3)cc2)c2ccc3c(c2)oc2ccccc23)c1 ; c1cc(-c2ccc3sc4ccccc4c3c2)cc(N(c2ccc(-c3cccc4sc5ccccc5c34)cc2)c2ccc3c(c2)oc2ccccc23)c1]

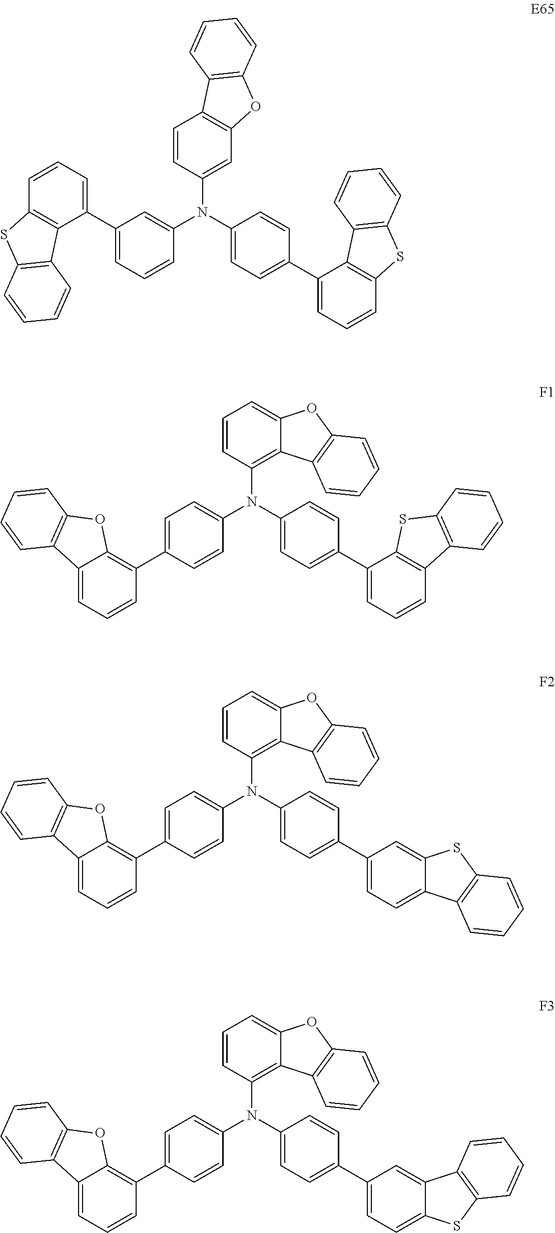 [SMILES: c1cc(-c2cccc3sc4ccccc4c23)cc(N(c2ccc(-c3cccc4sc5ccccc5c34)cc2)c2ccc3c(c2)oc2ccccc23)c1 ; c1ccc2c(c1)oc1c(-c3ccc(N(c4ccc(-c5cccc6c5sc5ccccc56)cc4)c4cccc5oc6ccccc6c45)cc3)cccc12 ; c1ccc2c(c1)oc1c(-c3ccc(N(c4ccc(-c5ccc6c(c5)sc5ccccc56)cc4)c4cccc5oc6ccccc6c45)cc3)cccc12 ; c1ccc2c(c1)oc1c(-c3ccc(N(c4ccc(-c5ccc6sc7ccccc7c6c5)cc4)c4cccc5oc6ccccc6c45)cc3)cccc12]

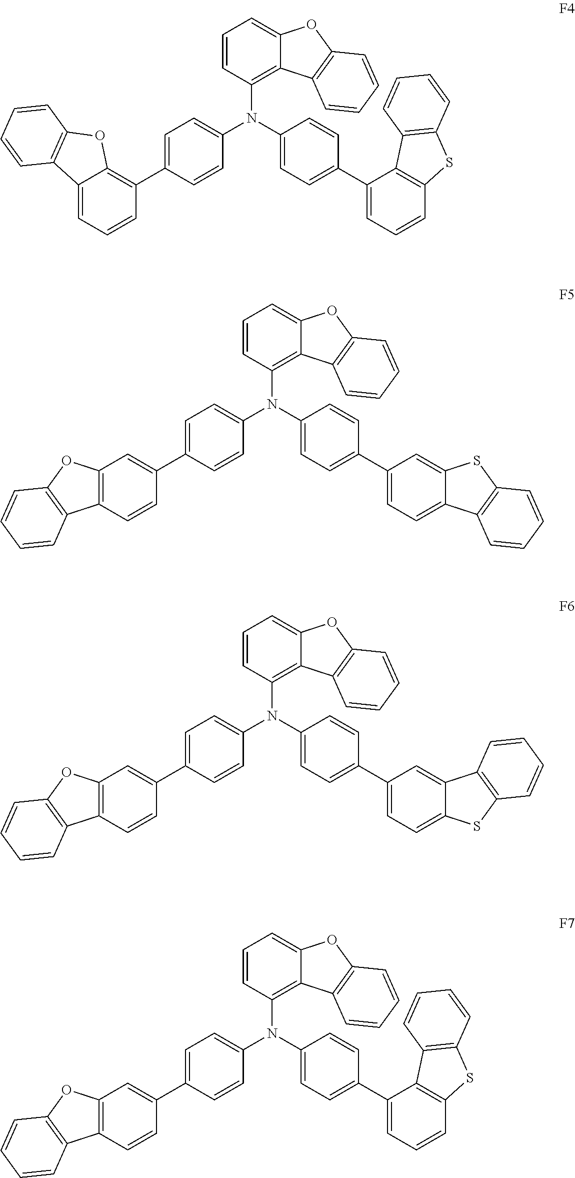 [SMILES: c1ccc2c(c1)oc1c(-c3ccc(N(c4ccc(-c5cccc6sc7ccccc7c56)cc4)c4cccc5oc6ccccc6c45)cc3)cccc12 ; c1ccc2c(c1)oc1cc(-c3ccc(N(c4ccc(-c5ccc6c(c5)sc5ccccc56)cc4)c4cccc5oc6ccccc6c45)cc3)ccc12 ; c1ccc2c(c1)oc1cc(-c3ccc(N(c4ccc(-c5ccc6sc7ccccc7c6c5)cc4)c4cccc5oc6ccccc6c45)cc3)ccc12 ; c1ccc2c(c1)oc1cc(-c3ccc(N(c4ccc(-c5cccc6sc7ccccc7c56)cc4)c4cccc5oc6ccccc6c45)cc3)ccc12]

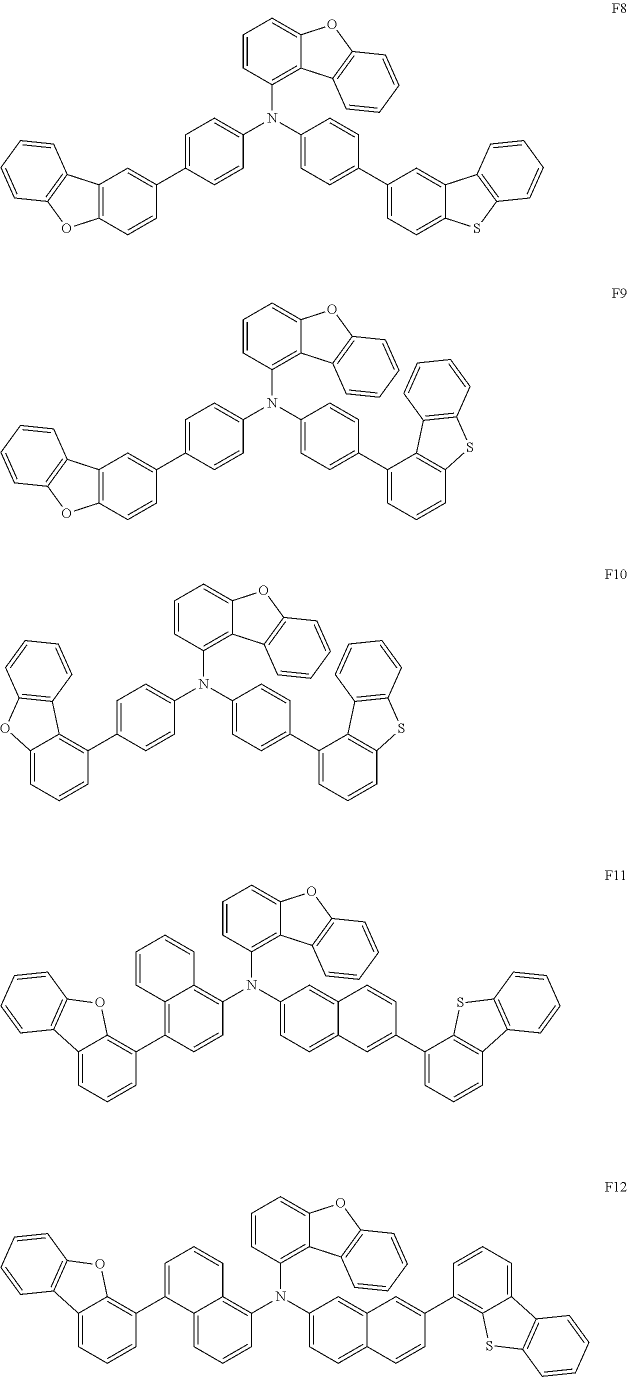 [SMILES: c1ccc2c(c1)oc1ccc(-c3ccc(N(c4ccc(-c5ccc6sc7ccccc7c6c5)cc4)c4cccc5oc6ccccc6c45)cc3)cc12 ; c1ccc2c(c1)oc1ccc(-c3ccc(N(c4ccc(-c5cccc6sc7ccccc7c56)cc4)c4cccc5oc6ccccc6c45)cc3)cc12 ; c1ccc2c(c1)oc1cccc(-c3ccc(N(c4ccc(-c5cccc6sc7ccccc7c56)cc4)c4cccc5oc6ccccc6c45)cc3)c12 ; c1ccc2c(c1)oc1c(-c3ccc(N(c4ccc5cc(-c6cccc7c6sc6ccccc67)ccc5c4)c4cccc5oc6ccccc6c45)c4ccccc34)cccc12 ; c1ccc2c(c1)oc1c(-c3cccc4c(N(c5ccc6ccc(-c7cccc8c7sc7ccccc78)cc6c5)c5cccc6oc7ccccc7c56)cccc34)cccc12]

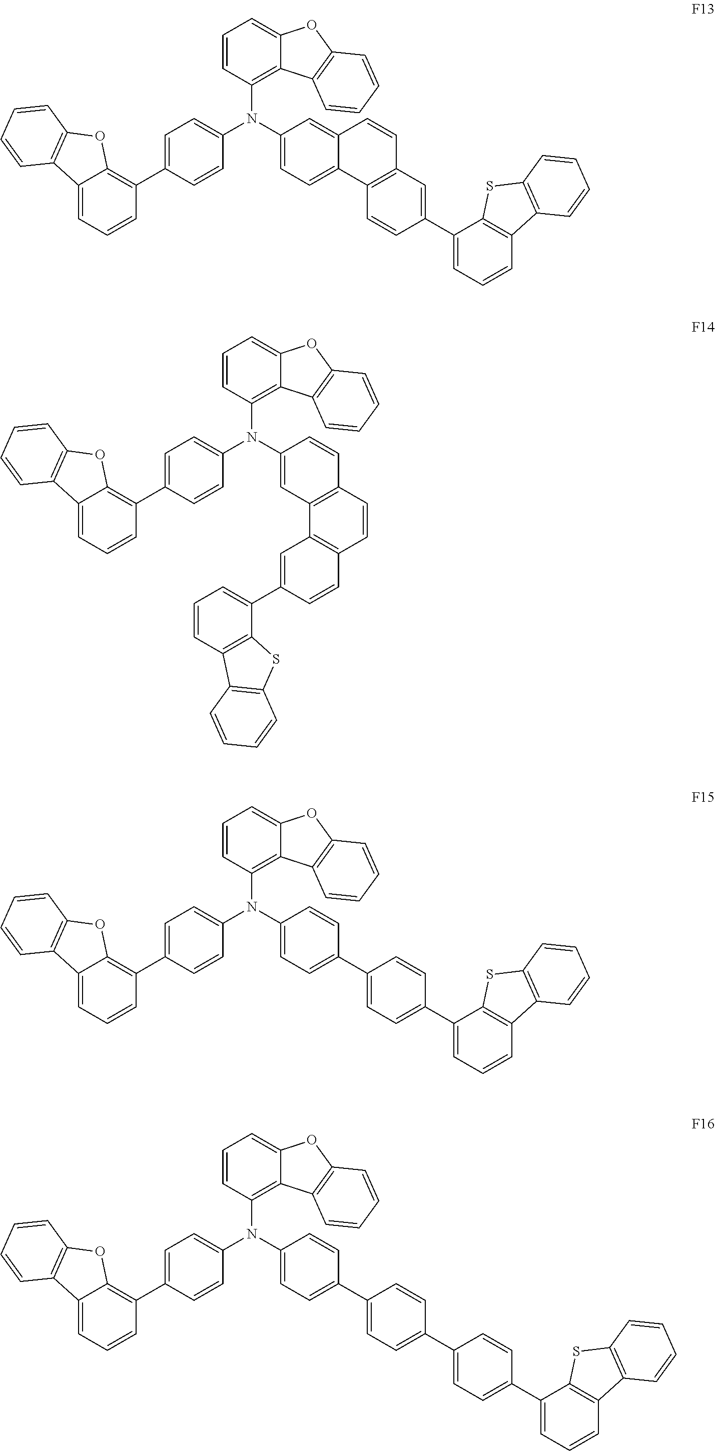 [SMILES: c1ccc2c(c1)oc1c(-c3ccc(N(c4ccc5c(ccc6cc(-c7cccc8c7sc7ccccc78)ccc65)c4)c4cccc5oc6ccccc6c45)cc3)cccc12 ; c1ccc2c(c1)oc1c(-c3ccc(N(c4ccc5ccc6ccc(-c7cccc8c7sc7ccccc78)cc6c5c4)c4cccc5oc6ccccc6c45)cc3)cccc12 ; c1ccc2c(c1)oc1c(-c3ccc(N(c4ccc(-c5ccc(-c6cccc7c6sc6ccccc67)cc5)cc4)c4cccc5oc6ccccc6c45)cc3)cccc12 ; c1ccc2c(c1)oc1c(-c3ccc(N(c4ccc(-c5ccc(-c6ccc(-c7cccc8c7sc7ccccc78)cc6)cc5)cc4)c4cccc5oc6ccccc6c45)cc3)cccc12]

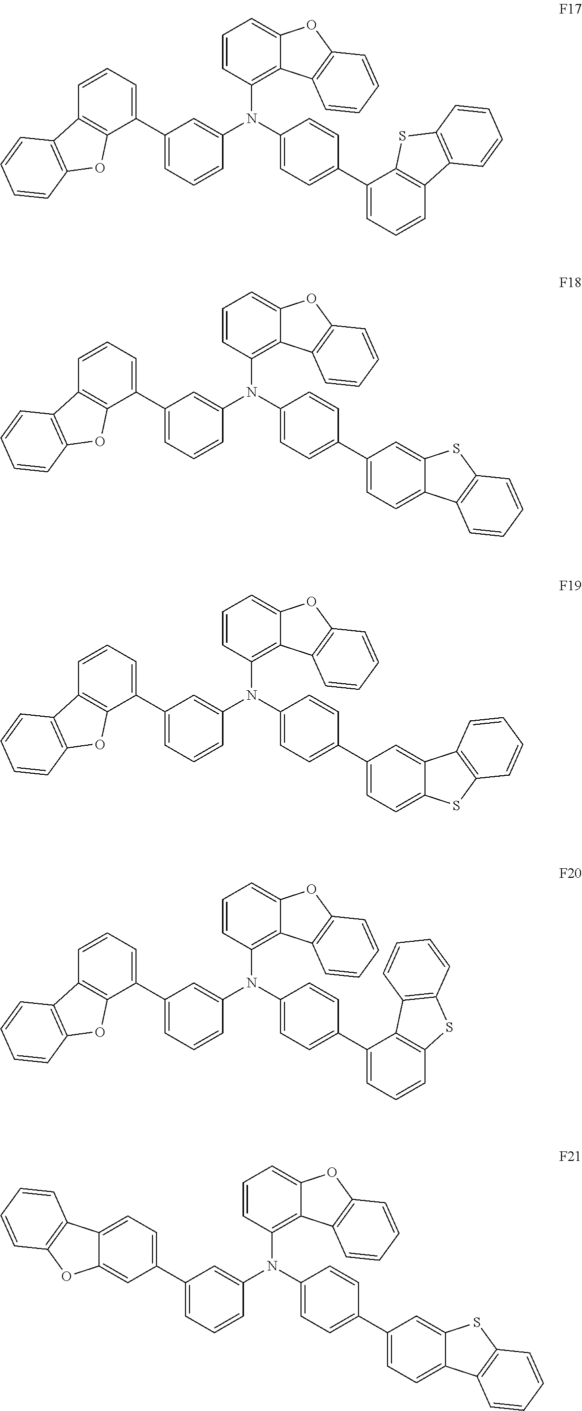 [SMILES: c1cc(-c2cccc3c2oc2ccccc23)cc(N(c2ccc(-c3cccc4c3sc3ccccc34)cc2)c2cccc3oc4ccccc4c23)c1 ; c1cc(-c2cccc3c2oc2ccccc23)cc(N(c2ccc(-c3ccc4c(c3)sc3ccccc34)cc2)c2cccc3oc4ccccc4c23)c1 ; c1cc(-c2cccc3c2oc2ccccc23)cc(N(c2ccc(-c3ccc4sc5ccccc5c4c3)cc2)c2cccc3oc4ccccc4c23)c1 ; c1cc(-c2cccc3c2oc2ccccc23)cc(N(c2ccc(-c3cccc4sc5ccccc5c34)cc2)c2cccc3oc4ccccc4c23)c1 ; c1cc(-c2ccc3c(c2)oc2ccccc23)cc(N(c2ccc(-c3ccc4c(c3)sc3ccccc34)cc2)c2cccc3oc4ccccc4c23)c1]

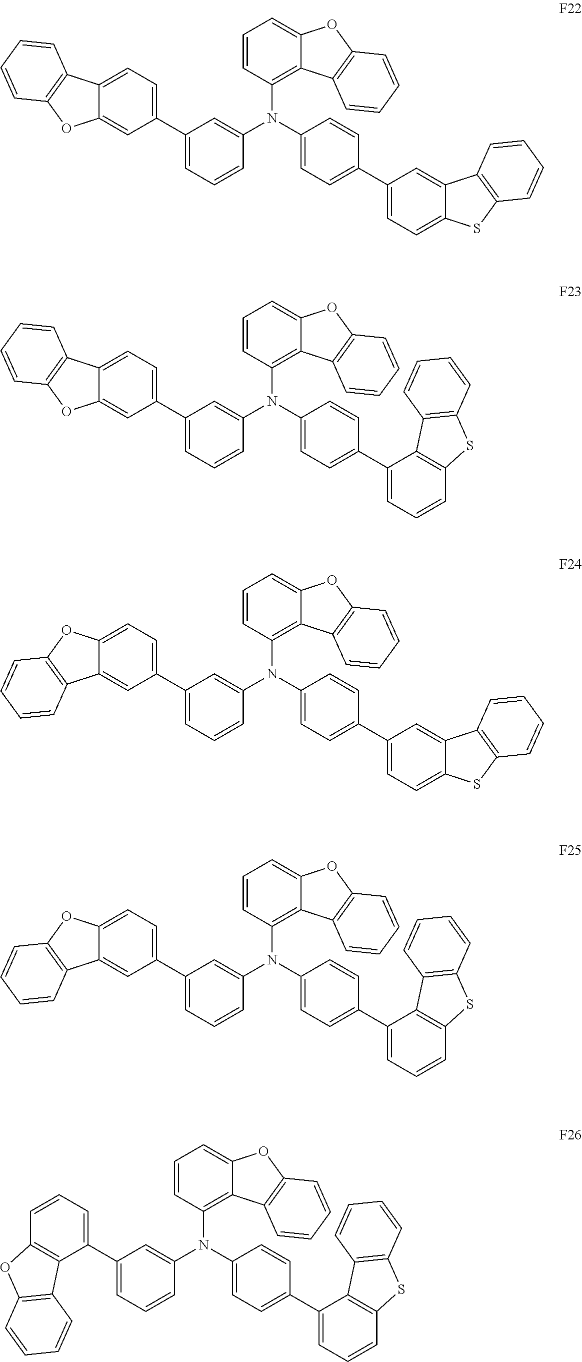 [SMILES: c1cc(-c2ccc3c(c2)oc2ccccc23)cc(N(c2ccc(-c3ccc4sc5ccccc5c4c3)cc2)c2cccc3oc4ccccc4c23)c1 ; c1cc(-c2ccc3c(c2)oc2ccccc23)cc(N(c2ccc(-c3cccc4sc5ccccc5c34)cc2)c2cccc3oc4ccccc4c23)c1 ; c1cc(-c2ccc3oc4ccccc4c3c2)cc(N(c2ccc(-c3ccc4sc5ccccc5c4c3)cc2)c2cccc3oc4ccccc4c23)c1 ; c1cc(-c2ccc3oc4ccccc4c3c2)cc(N(c2ccc(-c3cccc4sc5ccccc5c34)cc2)c2cccc3oc4ccccc4c23)c1 ; c1cc(-c2cccc3oc4ccccc4c23)cc(N(c2ccc(-c3cccc4sc5ccccc5c34)cc2)c2cccc3oc4ccccc4c23)c1]

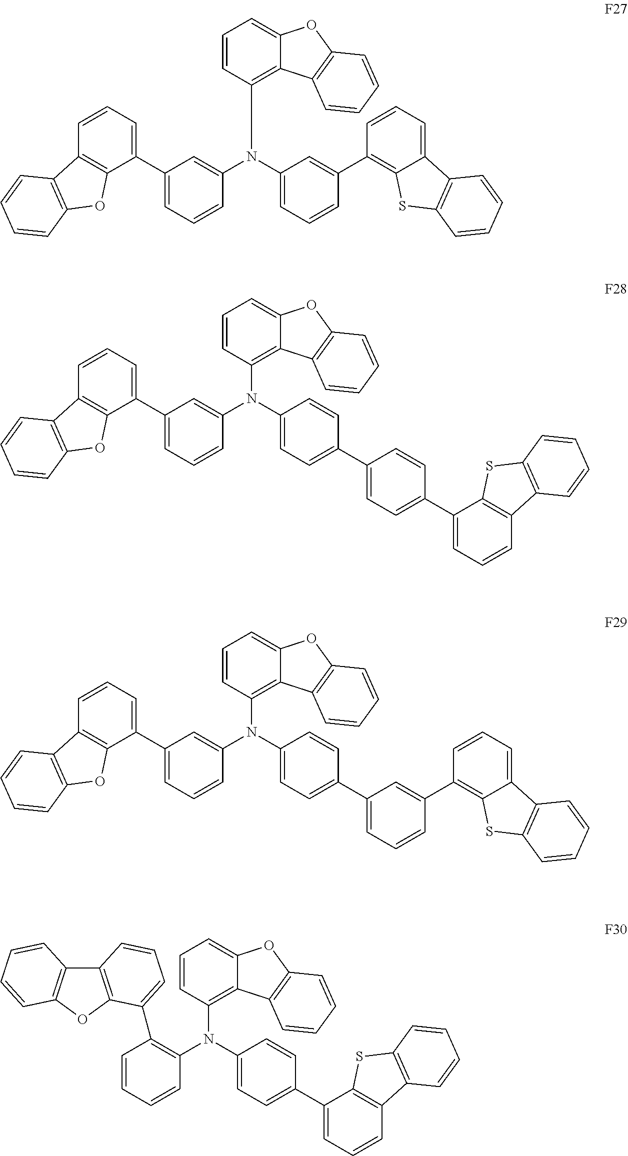 [SMILES: c1cc(-c2cccc3c2oc2ccccc23)cc(N(c2cccc(-c3cccc4c3sc3ccccc34)c2)c2cccc3oc4ccccc4c23)c1 ; c1cc(-c2cccc3c2oc2ccccc23)cc(N(c2ccc(-c3ccc(-c4cccc5c4sc4ccccc45)cc3)cc2)c2cccc3oc4ccccc4c23)c1 ; c1cc(-c2ccc(N(c3cccc(-c4cccc5c4oc4ccccc45)c3)c3cccc4oc5ccccc5c34)cc2)cc(-c2cccc3c2sc2ccccc23)c1 ; c1ccc(N(c2ccc(-c3cccc4c3sc3ccccc34)cc2)c2cccc3oc4ccccc4c23)c(-c2cccc3c2oc2ccccc23)c1]

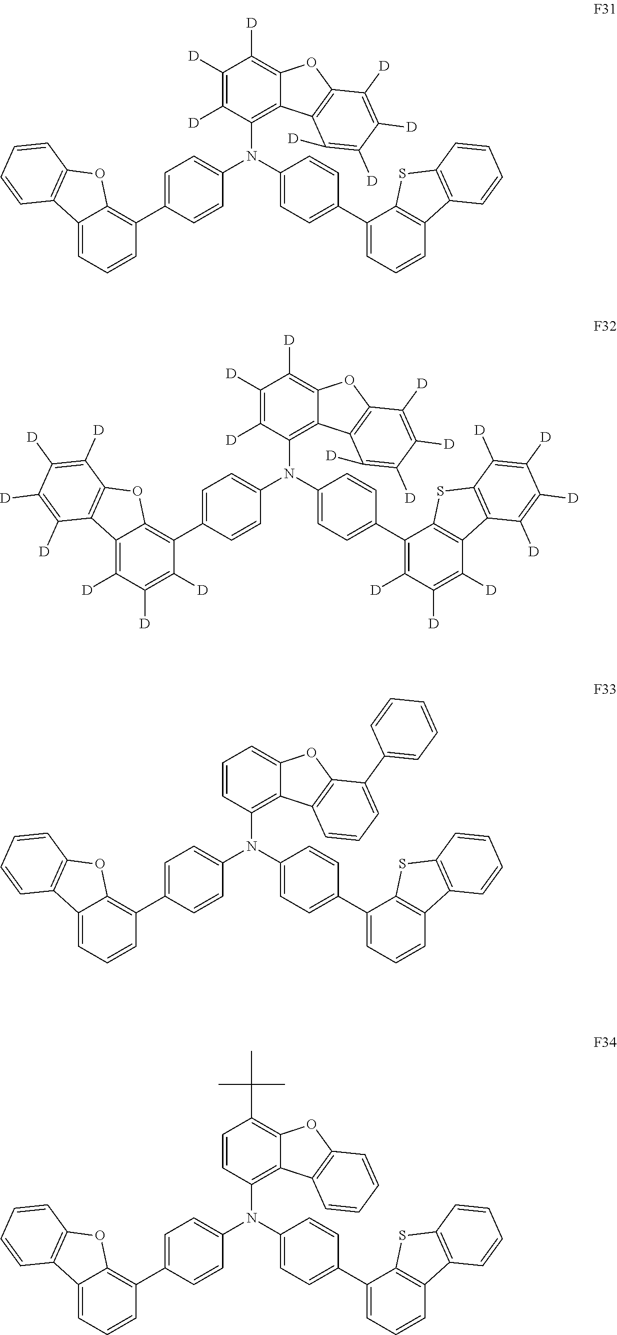

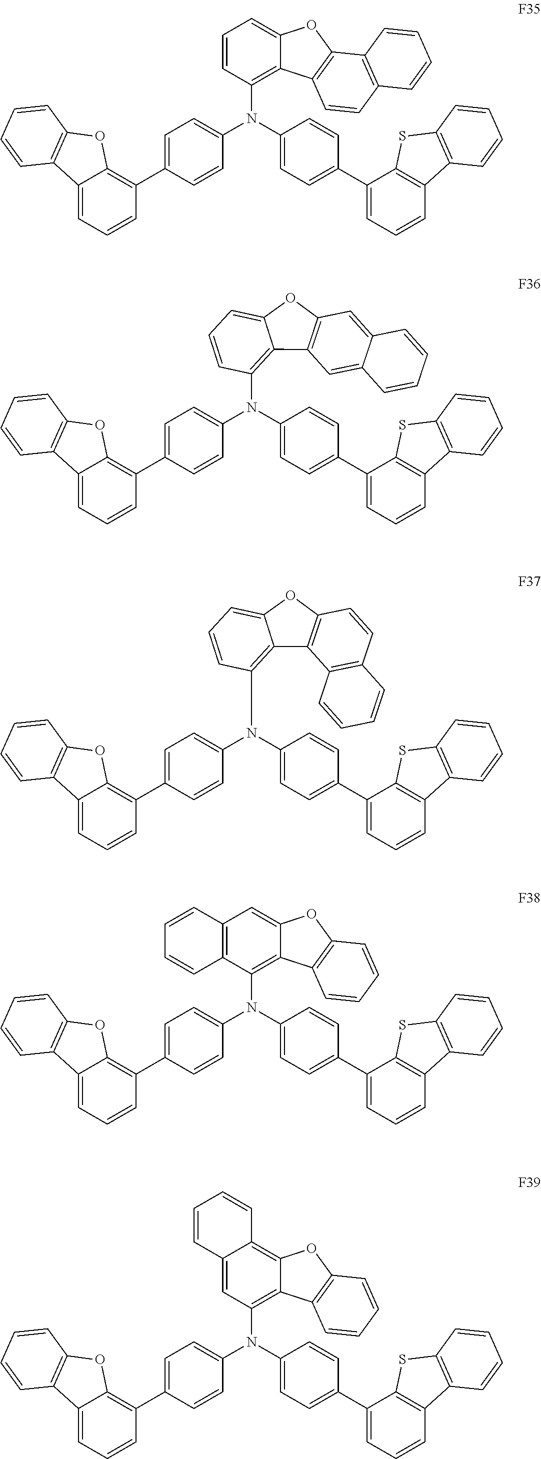 [SMILES: c1ccc2c(c1)ccc1c2oc2cccc(N(c3ccc(-c4cccc5c4oc4ccccc45)cc3)c3ccc(-c4cccc5c4sc4ccccc45)cc3)c21 ; c1ccc2cc3c(cc2c1)oc1cccc(N(c2ccc(-c4cccc5c4oc4ccccc45)cc2)c2ccc(-c4cccc5c4sc4ccccc45)cc2)c13 ; c1ccc2c(c1)ccc1oc3cccc(N(c4ccc(-c5cccc6c5oc5ccccc56)cc4)c4ccc(-c5cccc6c5sc5ccccc56)cc4)c3c12 ; c1ccc2c(N(c3ccc(-c4cccc5c4oc4ccccc45)cc3)c3ccc(-c4cccc5c4sc4ccccc45)cc3)c3c(cc2c1)oc1ccccc13 ; c1ccc2c(c1)cc(N(c1ccc(-c3cccc4c3oc3ccccc34)cc1)c1ccc(-c3cccc4c3sc3ccccc34)cc1)c1c3ccccc3oc21]

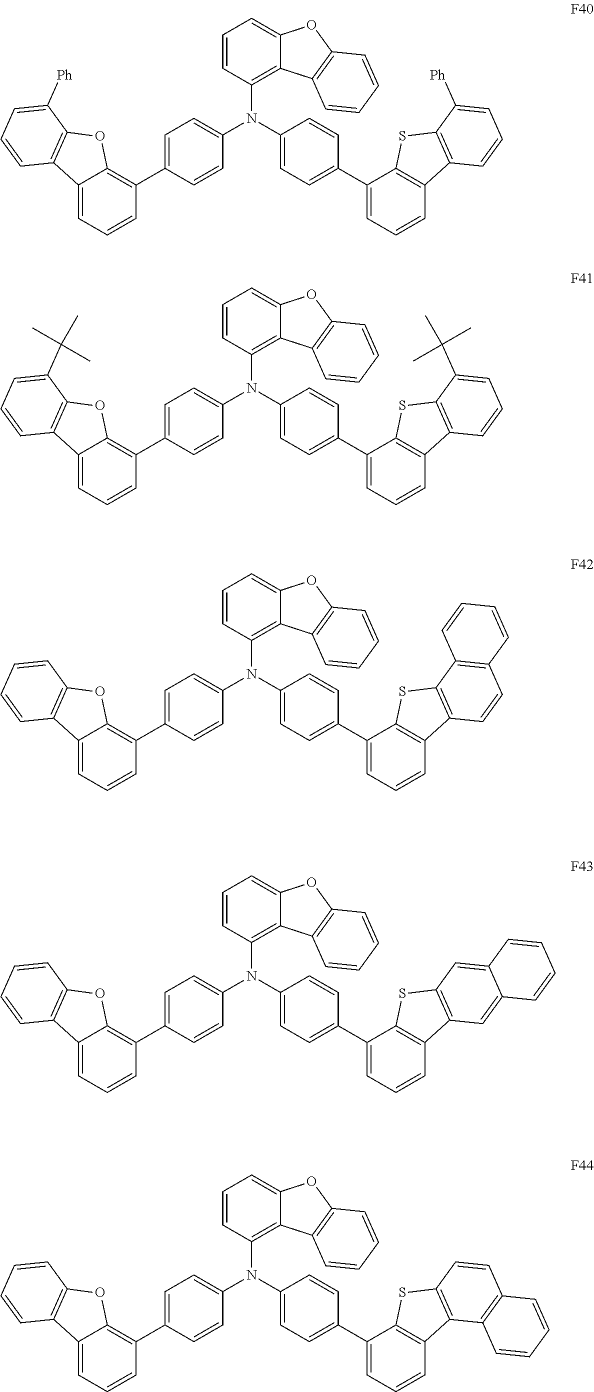 [SMILES: c1ccc(-c2cccc3c2oc2c(-c4ccc(N(c5ccc(-c6cccc7c6sc6c(-c8ccccc8)cccc67)cc5)c5cccc6oc7ccccc7c56)cc4)cccc23)cc1 ; CC(C)(C)c1cccc2c1oc1c(-c3ccc(N(c4ccc(-c5cccc6c5sc5c(C(C)(C)C)cccc56)cc4)c4cccc5oc6ccccc6c45)cc3)cccc12 ; c1ccc2c(c1)ccc1c3cccc(-c4ccc(N(c5ccc(-c6cccc7c6oc6ccccc67)cc5)c5cccc6oc7ccccc7c56)cc4)c3sc21 ; c1ccc2cc3c(cc2c1)sc1c(-c2ccc(N(c4ccc(-c5cccc6c5oc5ccccc56)cc4)c4cccc5oc6ccccc6c45)cc2)cccc13 ; c1ccc2c(c1)ccc1sc3c(-c4ccc(N(c5ccc(-c6cccc7c6oc6ccccc67)cc5)c5cccc6oc7ccccc7c56)cc4)cccc3c12]

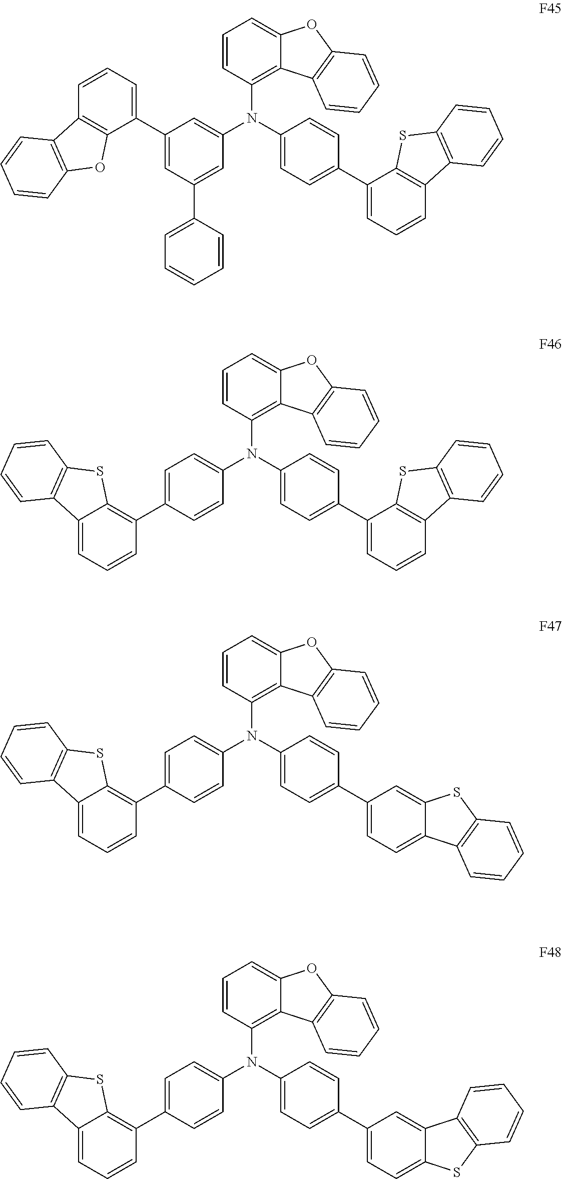 [SMILES: c1ccc(-c2cc(-c3cccc4c3oc3ccccc34)cc(N(c3ccc(-c4cccc5c4sc4ccccc45)cc3)c3cccc4oc5ccccc5c34)c2)cc1 ; c1ccc2c(c1)oc1cccc(N(c3ccc(-c4cccc5c4sc4ccccc45)cc3)c3ccc(-c4cccc5c4sc4ccccc45)cc3)c12 ; c1ccc2c(c1)oc1cccc(N(c3ccc(-c4ccc5c(c4)sc4ccccc45)cc3)c3ccc(-c4cccc5c4sc4ccccc45)cc3)c12 ; c1ccc2c(c1)oc1cccc(N(c3ccc(-c4ccc5sc6ccccc6c5c4)cc3)c3ccc(-c4cccc5c4sc4ccccc45)cc3)c12]

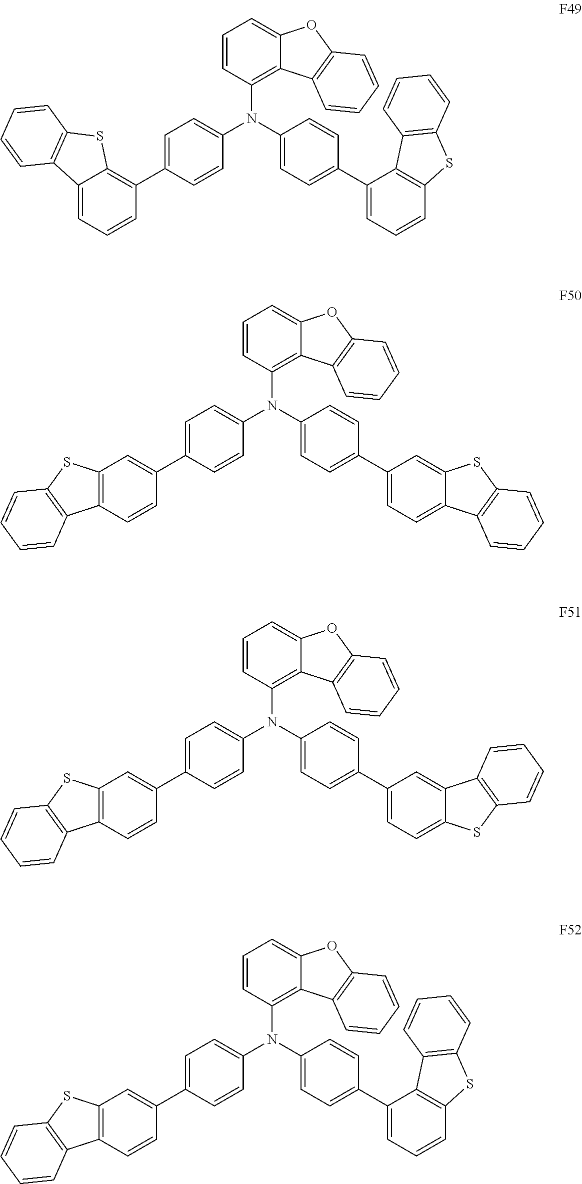 [SMILES: c1ccc2c(c1)oc1cccc(N(c3ccc(-c4cccc5c4sc4ccccc45)cc3)c3ccc(-c4cccc5sc6ccccc6c45)cc3)c12 ; c1ccc2c(c1)oc1cccc(N(c3ccc(-c4ccc5c(c4)sc4ccccc45)cc3)c3ccc(-c4ccc5c(c4)sc4ccccc45)cc3)c12 ; c1ccc2c(c1)oc1cccc(N(c3ccc(-c4ccc5c(c4)sc4ccccc45)cc3)c3ccc(-c4ccc5sc6ccccc6c5c4)cc3)c12 ; c1ccc2c(c1)oc1cccc(N(c3ccc(-c4ccc5c(c4)sc4ccccc45)cc3)c3ccc(-c4cccc5sc6ccccc6c45)cc3)c12]

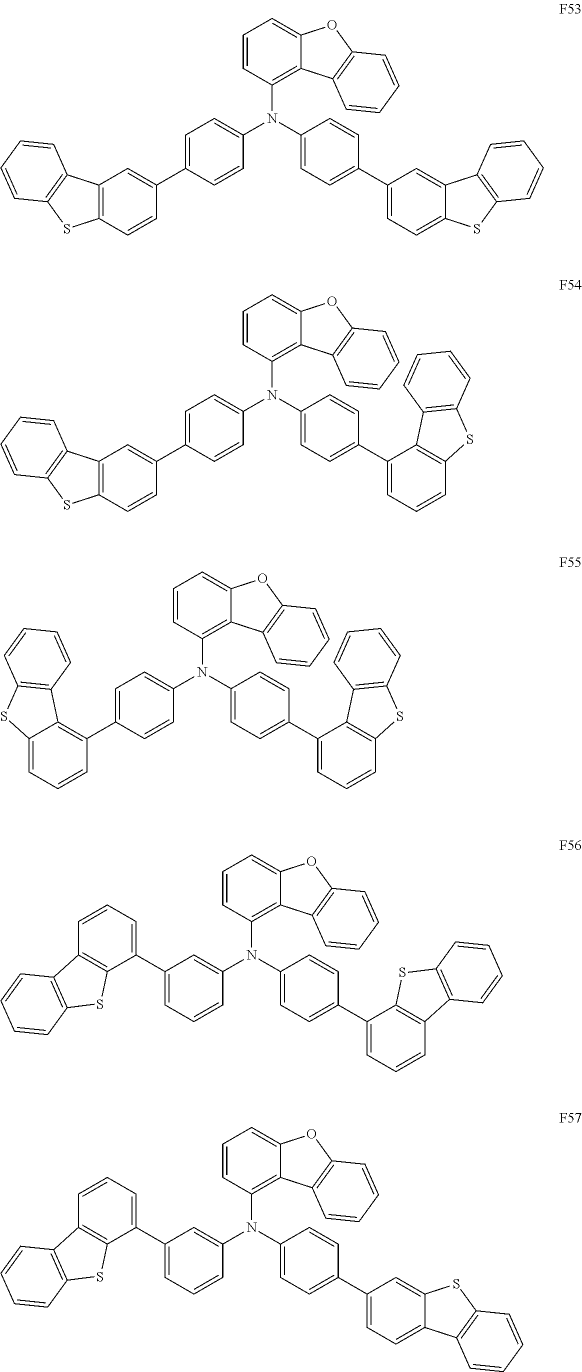 [SMILES: c1ccc2c(c1)oc1cccc(N(c3ccc(-c4ccc5sc6ccccc6c5c4)cc3)c3ccc(-c4ccc5sc6ccccc6c5c4)cc3)c12 ; c1ccc2c(c1)oc1cccc(N(c3ccc(-c4ccc5sc6ccccc6c5c4)cc3)c3ccc(-c4cccc5sc6ccccc6c45)cc3)c12 ; c1ccc2c(c1)oc1cccc(N(c3ccc(-c4cccc5sc6ccccc6c45)cc3)c3ccc(-c4cccc5sc6ccccc6c45)cc3)c12 ; c1cc(-c2cccc3c2sc2ccccc23)cc(N(c2ccc(-c3cccc4c3sc3ccccc34)cc2)c2cccc3oc4ccccc4c23)c1 ; c1cc(-c2cccc3c2sc2ccccc23)cc(N(c2ccc(-c3ccc4c(c3)sc3ccccc34)cc2)c2cccc3oc4ccccc4c23)c1]

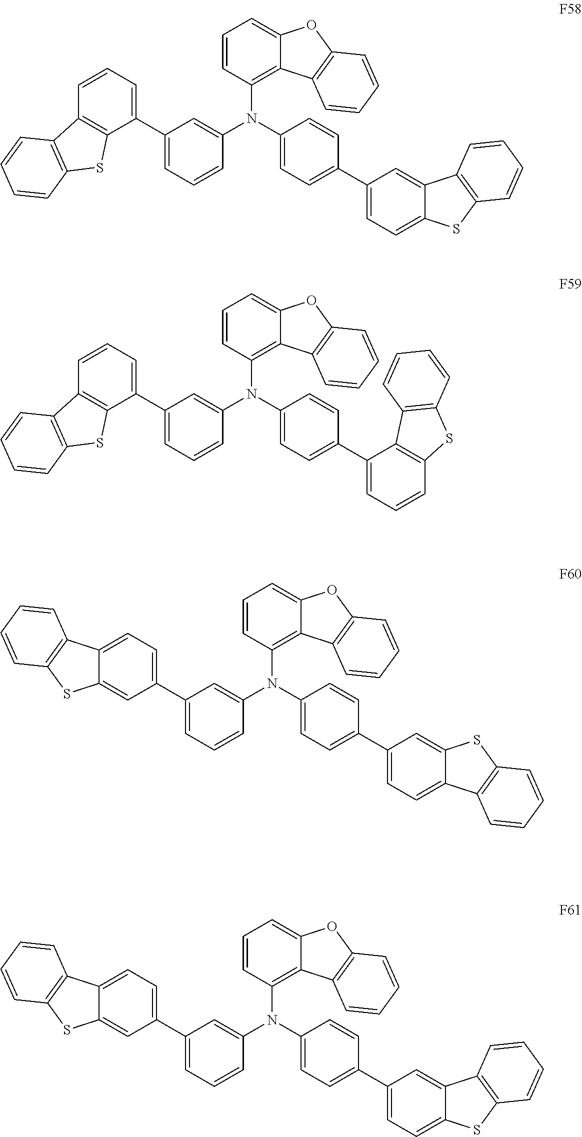 [SMILES: c1cc(-c2cccc3c2sc2ccccc23)cc(N(c2ccc(-c3ccc4sc5ccccc5c4c3)cc2)c2cccc3oc4ccccc4c23)c1 ; c1cc(-c2cccc3c2sc2ccccc23)cc(N(c2ccc(-c3cccc4sc5ccccc5c34)cc2)c2cccc3oc4ccccc4c23)c1 ; c1cc(-c2ccc3c(c2)sc2ccccc23)cc(N(c2ccc(-c3ccc4c(c3)sc3ccccc34)cc2)c2cccc3oc4ccccc4c23)c1 ; c1cc(-c2ccc3c(c2)sc2ccccc23)cc(N(c2ccc(-c3ccc4sc5ccccc5c4c3)cc2)c2cccc3oc4ccccc4c23)c1]

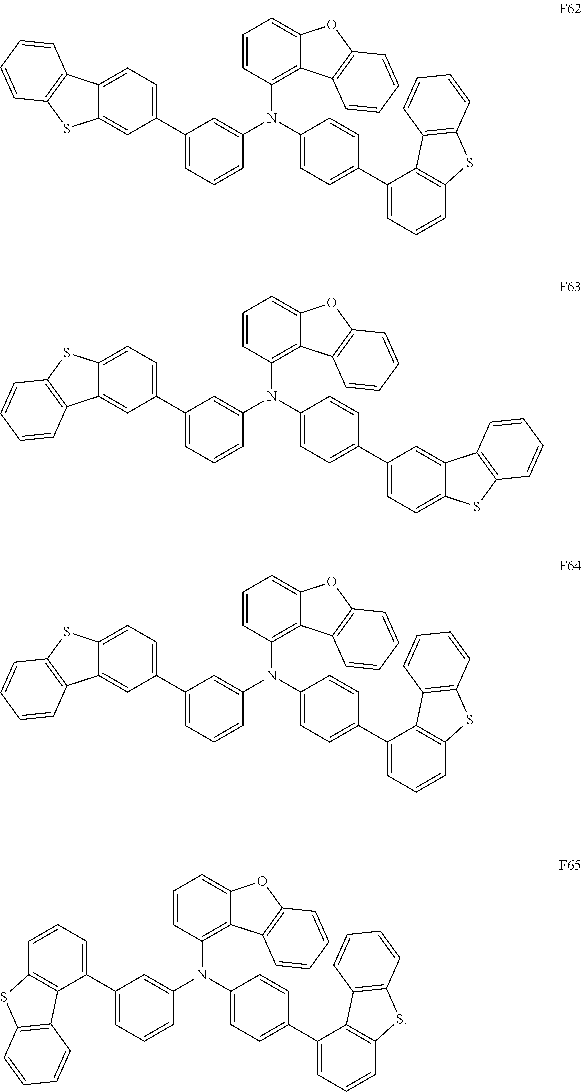 [SMILES: c1cc(-c2ccc3c(c2)sc2ccccc23)cc(N(c2ccc(-c3cccc4sc5ccccc5c34)cc2)c2cccc3oc4ccccc4c23)c1 ; c1cc(-c2ccc3sc4ccccc4c3c2)cc(N(c2ccc(-c3ccc4sc5ccccc5c4c3)cc2)c2cccc3oc4ccccc4c23)c1 ; c1cc(-c2ccc3sc4ccccc4c3c2)cc(N(c2ccc(-c3cccc4sc5ccccc5c34)cc2)c2cccc3oc4ccccc4c23)c1 ; c1cc(-c2cccc3sc4ccccc4c23)cc(N(c2ccc(-c3cccc4sc5ccccc5c34)cc2)c2cccc3oc4ccccc4c23)c1]

12. An amine compound represented by Formula 1: 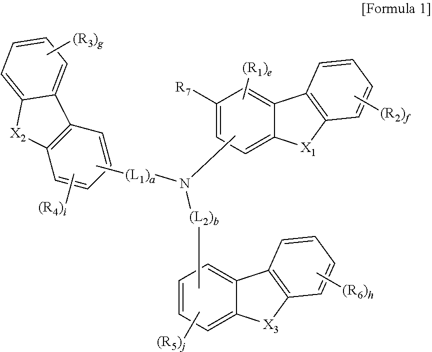
wherein in Formula 1,
X₁, X₂, and X₃ are each independently O or S,
R₁ to R₆ are each independently a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, or are combined with an adjacent group to form a ring,
R₇ is a hydrogen atom, a deuterium atom, a halogen atom, or a substituted or unsubstituted alkyl group of 1 to 20 carbon atoms,
L₁ and L₂ are each independently a substituted or unsubstituted arylene group of 6 to 30 ring-forming carbon atoms, where a heteroaryl group is excluded,
a and b are each independently an integer from 1 to 3,
e is an integer from 0 to 2,
f to h are each independently an integer from 0 to 4,
i and j are each independently an integer from 0 to 3, and
X₁, X₂, and X₃ are not O at the same time.

13. The amine compound of claim 12, wherein Formula 1 is represented by Formula 2: 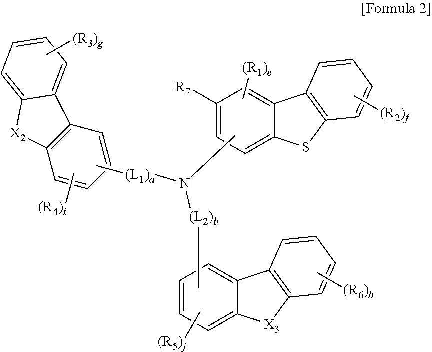
wherein in Formula 2,
X₂, X₃, R₁ to R₇, L₁, L₂, a, b, and e to j are the same as defined in connection with Formula 1.

14. The amine compound of claim 12, wherein Formula 1 is represented by Formula 3: 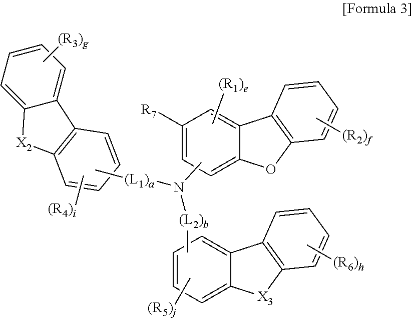
wherein in Formula 3,
X₂, X₃, R₁ to R₇, L₁, L₂, a, b, and e to j are the same as defined in connection with Formula 1.

15. The amine compound of claim 13, wherein Formula 2 is represented by one of Formula 4-1 to Formula 4-3: 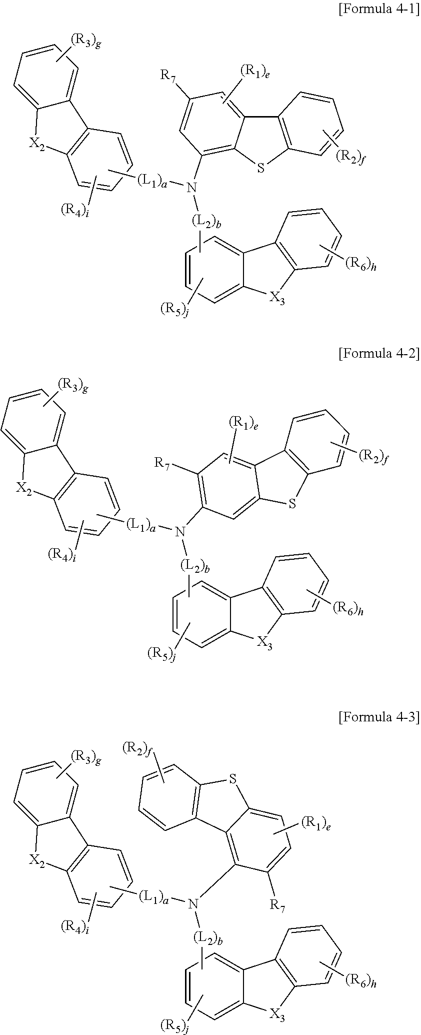
wherein in Formula 4-1 to Formula 4-3,
X₂, X₃, R₁ to R₇, L₁, L₂, a, b, and e to j are the same as defined in connection with Formula 2.

16. The amine compound of claim 14, wherein Formula 3 is represented by one of Formula 5-1 to Formula 5-3: 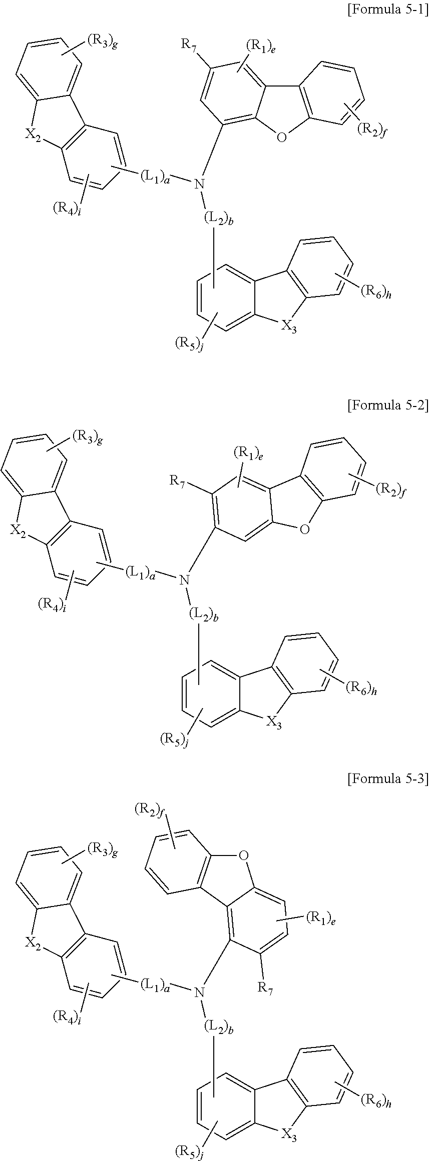
wherein in Formula 5-1 to Formula 5-3,
X₂, X₃, R₁ to R₇, L₁, L₂, a, b, and e to j are the same as defined in connection with Formula 3.

17. The amine compound of claim 12, wherein
a and b are each 1, and
L₁ and L₂ are each independently a substituted or unsubstituted phenylene group, a substituted or unsubstituted biphenylene group, a substituted or unsubstituted terphenyl group, a substituted or unsubstituted naphthylene group, or a substituted or unsubstituted phenanthrylene group.

18. The amine compound of claim 12, wherein
a and b are each 1, and
L₁ and L₂ are each independently represented by one of L-1 to L-11: 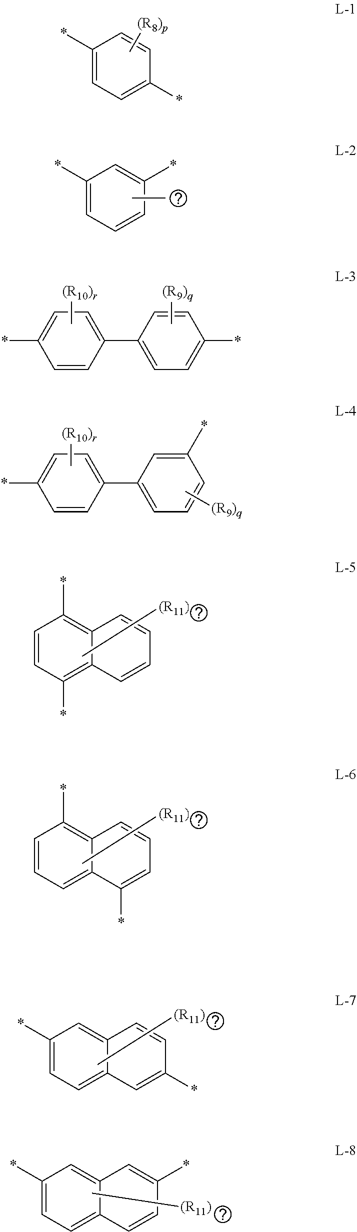 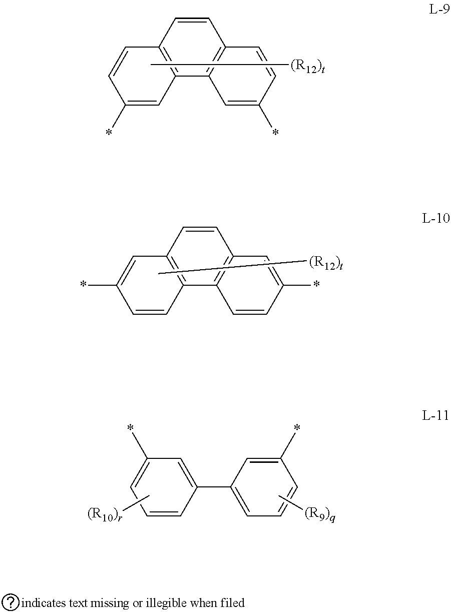
wherein in L-1 to L-11,
R₈ to R₁₂ are each independently a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms,
p to r are each independently an integer from 0 to 4,
s is an integer from 0 to 6,
t is an integer from 0 to 8, and
* indicates a binding site to a neighboring atom.

19. The amine compound of claim 12, wherein the amine compound represented by Formula 1 is selected from Compound Group 1:

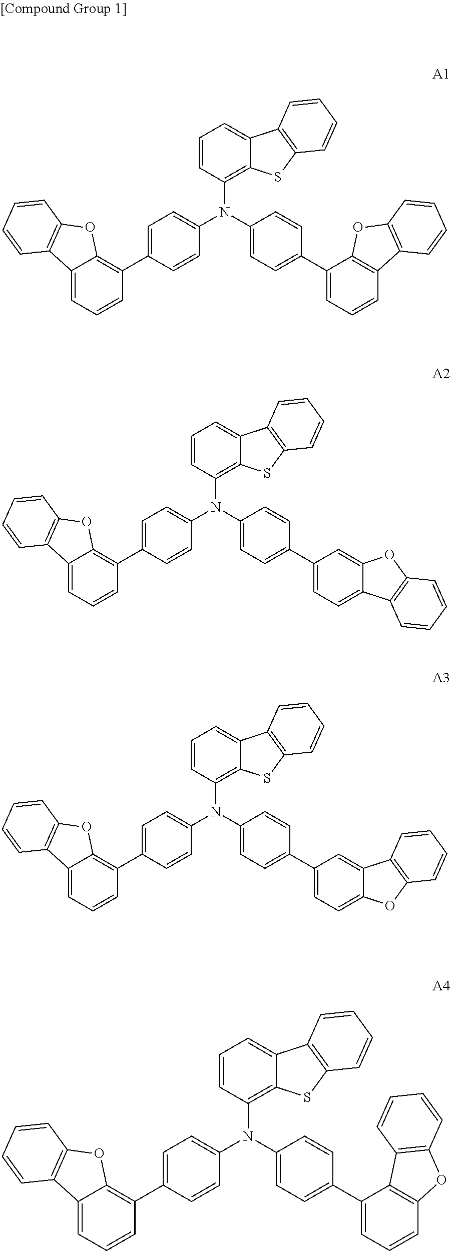 [SMILES: c1ccc2c(c1)oc1c(-c3ccc(N(c4ccc(-c5cccc6c5oc5ccccc56)cc4)c4cccc5c4sc4ccccc45)cc3)cccc12 ; c1ccc2c(c1)oc1cc(-c3ccc(N(c4ccc(-c5cccc6c5oc5ccccc56)cc4)c4cccc5c4sc4ccccc45)cc3)ccc12 ; c1ccc2c(c1)oc1ccc(-c3ccc(N(c4ccc(-c5cccc6c5oc5ccccc56)cc4)c4cccc5c4sc4ccccc45)cc3)cc12 ; c1ccc2c(c1)oc1c(-c3ccc(N(c4ccc(-c5cccc6oc7ccccc7c56)cc4)c4cccc5c4sc4ccccc45)cc3)cccc12]

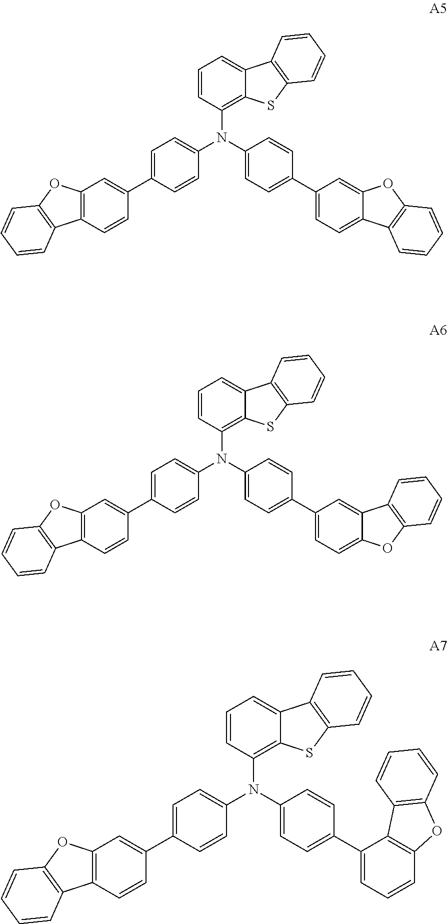 [SMILES: c1ccc2c(c1)oc1cc(-c3ccc(N(c4ccc(-c5ccc6c(c5)oc5ccccc56)cc4)c4cccc5c4sc4ccccc45)cc3)ccc12 ; c1ccc2c(c1)oc1cc(-c3ccc(N(c4ccc(-c5ccc6oc7ccccc7c6c5)cc4)c4cccc5c4sc4ccccc45)cc3)ccc12 ; c1ccc2c(c1)oc1cc(-c3ccc(N(c4ccc(-c5cccc6oc7ccccc7c56)cc4)c4cccc5c4sc4ccccc45)cc3)ccc12]

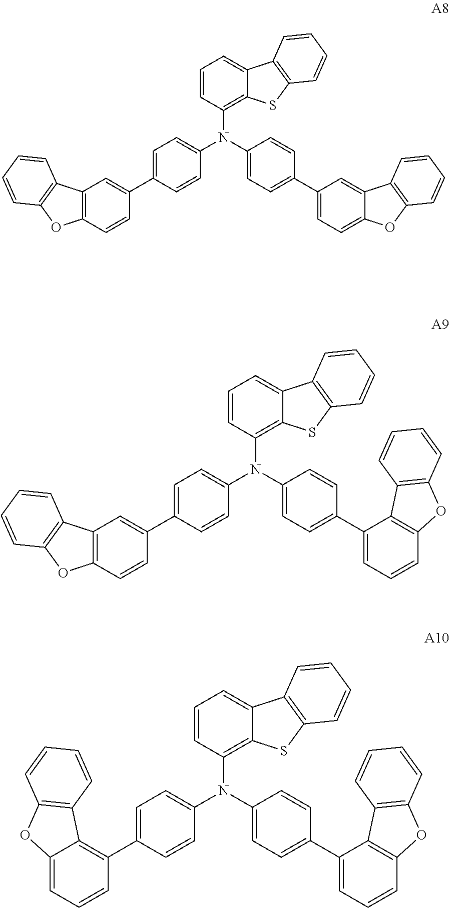 [SMILES: c1ccc2c(c1)oc1ccc(-c3ccc(N(c4ccc(-c5ccc6oc7ccccc7c6c5)cc4)c4cccc5c4sc4ccccc45)cc3)cc12 ; c1ccc2c(c1)oc1ccc(-c3ccc(N(c4ccc(-c5cccc6oc7ccccc7c56)cc4)c4cccc5c4sc4ccccc45)cc3)cc12 ; c1ccc2c(c1)oc1cccc(-c3ccc(N(c4ccc(-c5cccc6oc7ccccc7c56)cc4)c4cccc5c4sc4ccccc45)cc3)c12]

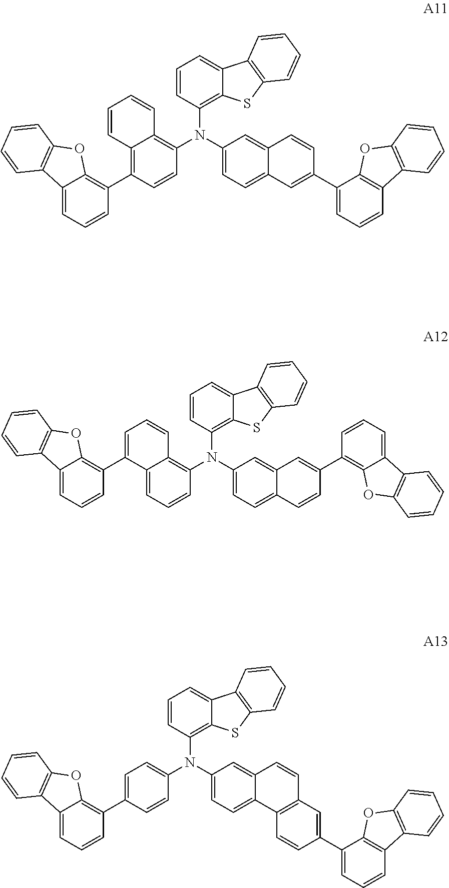 [SMILES: c1ccc2c(c1)oc1c(-c3ccc4cc(N(c5ccc(-c6cccc7c6oc6ccccc67)c6ccccc56)c5cccc6c5sc5ccccc56)ccc4c3)cccc12 ; c1ccc2c(c1)oc1c(-c3ccc4ccc(N(c5cccc6c(-c7cccc8c7oc7ccccc78)cccc56)c5cccc6c5sc5ccccc56)cc4c3)cccc12 ; c1ccc2c(c1)oc1c(-c3ccc(N(c4ccc5c(ccc6cc(-c7cccc8c7oc7ccccc78)ccc65)c4)c4cccc5c4sc4ccccc45)cc3)cccc12]

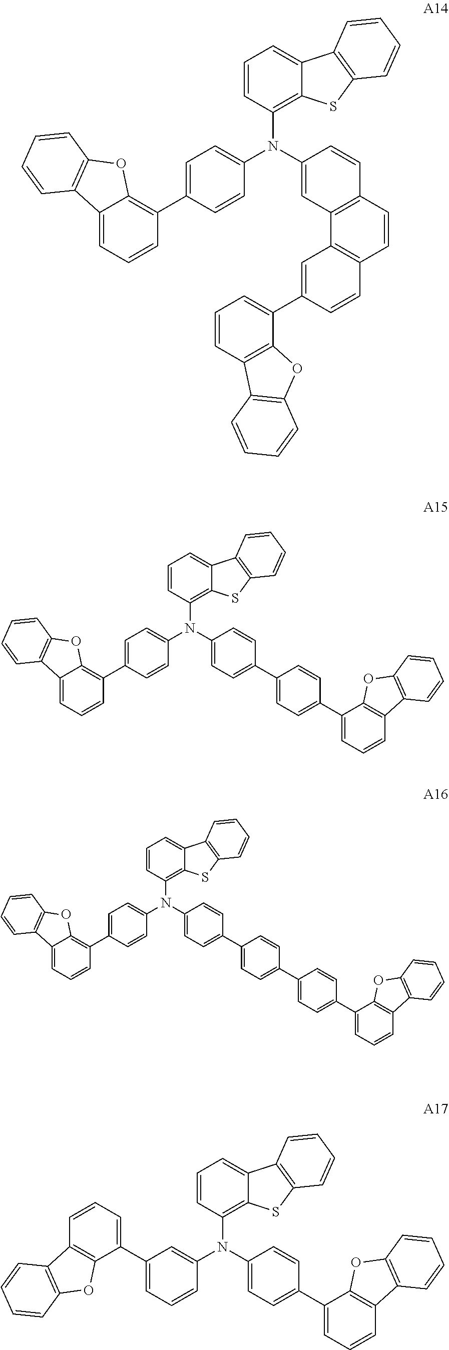 [SMILES: c1ccc2c(c1)oc1c(-c3ccc(N(c4ccc5ccc6ccc(-c7cccc8c7oc7ccccc78)cc6c5c4)c4cccc5c4sc4ccccc45)cc3)cccc12 ; c1ccc2c(c1)oc1c(-c3ccc(-c4ccc(N(c5ccc(-c6cccc7c6oc6ccccc67)cc5)c5cccc6c5sc5ccccc56)cc4)cc3)cccc12 ; c1ccc2c(c1)oc1c(-c3ccc(-c4ccc(-c5ccc(N(c6ccc(-c7cccc8c7oc7ccccc78)cc6)c6cccc7c6sc6ccccc67)cc5)cc4)cc3)cccc12 ; c1cc(-c2cccc3c2oc2ccccc23)cc(N(c2ccc(-c3cccc4c3oc3ccccc34)cc2)c2cccc3c2sc2ccccc23)c1]

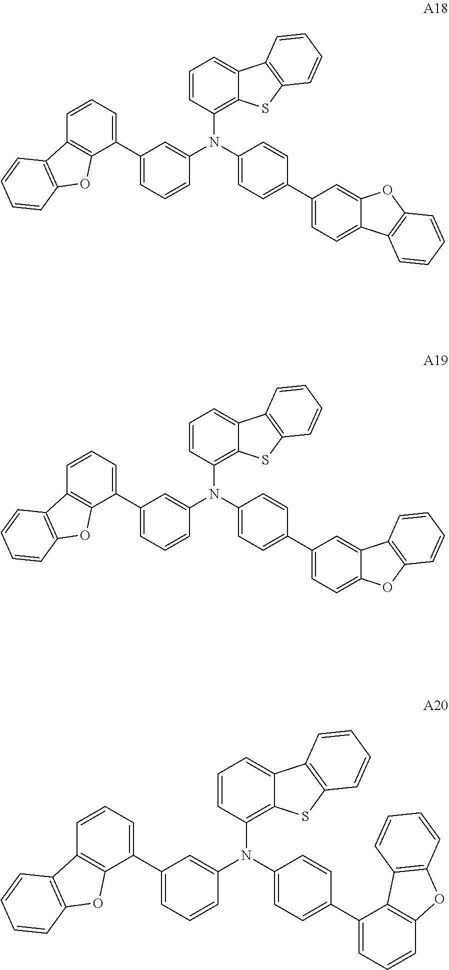 [SMILES: c1cc(-c2cccc3c2oc2ccccc23)cc(N(c2ccc(-c3ccc4c(c3)oc3ccccc34)cc2)c2cccc3c2sc2ccccc23)c1 ; c1cc(-c2cccc3c2oc2ccccc23)cc(N(c2ccc(-c3ccc4oc5ccccc5c4c3)cc2)c2cccc3c2sc2ccccc23)c1 ; c1cc(-c2cccc3c2oc2ccccc23)cc(N(c2ccc(-c3cccc4oc5ccccc5c34)cc2)c2cccc3c2sc2ccccc23)c1]

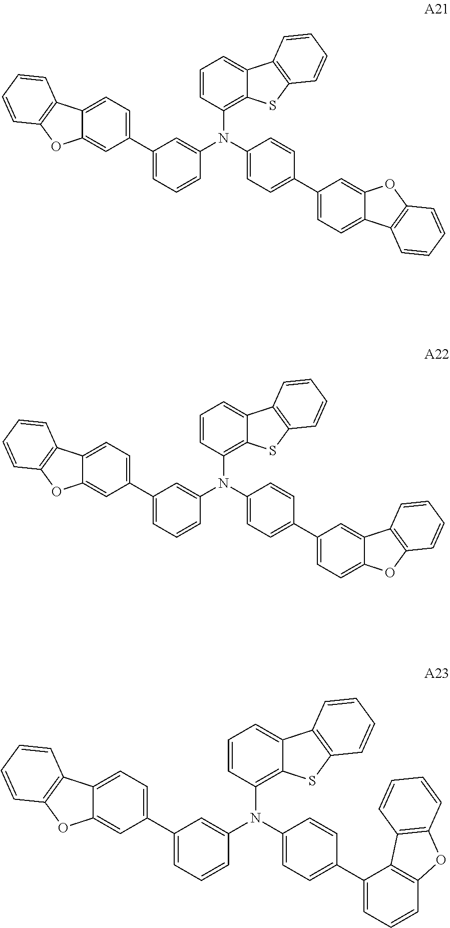 [SMILES: c1cc(-c2ccc3c(c2)oc2ccccc23)cc(N(c2ccc(-c3ccc4c(c3)oc3ccccc34)cc2)c2cccc3c2sc2ccccc23)c1 ; c1cc(-c2ccc3c(c2)oc2ccccc23)cc(N(c2ccc(-c3ccc4oc5ccccc5c4c3)cc2)c2cccc3c2sc2ccccc23)c1 ; c1cc(-c2ccc3c(c2)oc2ccccc23)cc(N(c2ccc(-c3cccc4oc5ccccc5c34)cc2)c2cccc3c2sc2ccccc23)c1]

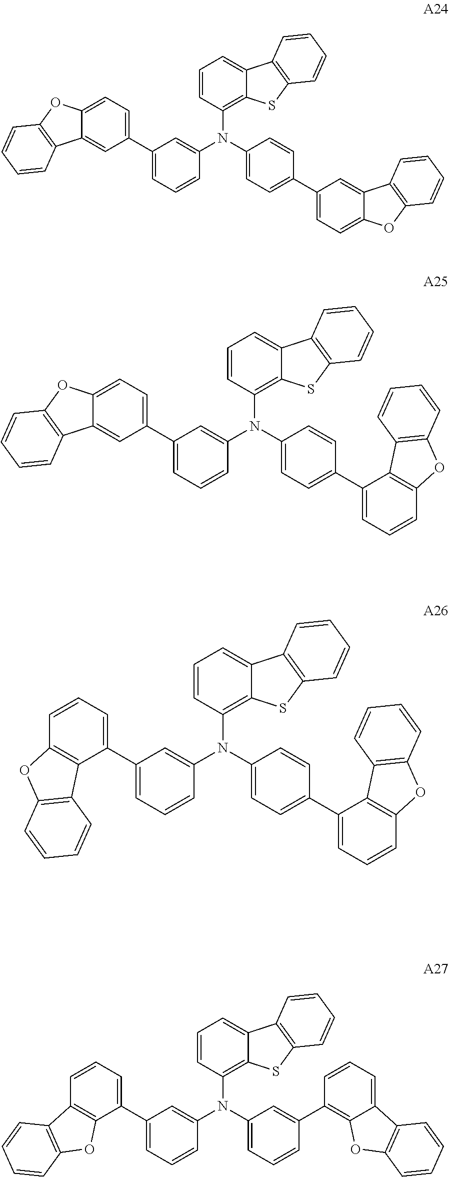 [SMILES: c1cc(-c2ccc3oc4ccccc4c3c2)cc(N(c2ccc(-c3ccc4oc5ccccc5c4c3)cc2)c2cccc3c2sc2ccccc23)c1 ; c1cc(-c2ccc3oc4ccccc4c3c2)cc(N(c2ccc(-c3cccc4oc5ccccc5c34)cc2)c2cccc3c2sc2ccccc23)c1 ; c1cc(-c2cccc3oc4ccccc4c23)cc(N(c2ccc(-c3cccc4oc5ccccc5c34)cc2)c2cccc3c2sc2ccccc23)c1 ; c1cc(-c2cccc3c2oc2ccccc23)cc(N(c2cccc(-c3cccc4c3oc3ccccc34)c2)c2cccc3c2sc2ccccc23)c1]

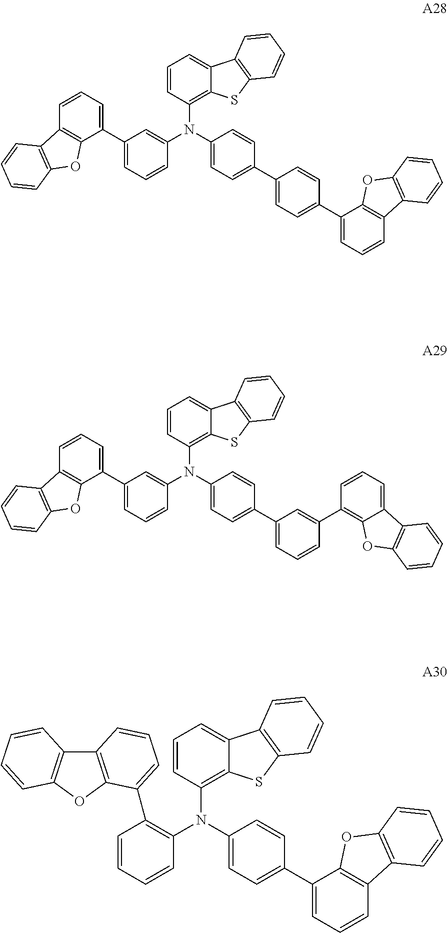 [SMILES: c1cc(-c2cccc3c2oc2ccccc23)cc(N(c2ccc(-c3ccc(-c4cccc5c4oc4ccccc45)cc3)cc2)c2cccc3c2sc2ccccc23)c1 ; c1cc(-c2ccc(N(c3cccc(-c4cccc5c4oc4ccccc45)c3)c3cccc4c3sc3ccccc34)cc2)cc(-c2cccc3c2oc2ccccc23)c1 ; c1ccc(N(c2ccc(-c3cccc4c3oc3ccccc34)cc2)c2cccc3c2sc2ccccc23)c(-c2cccc3c2oc2ccccc23)c1]

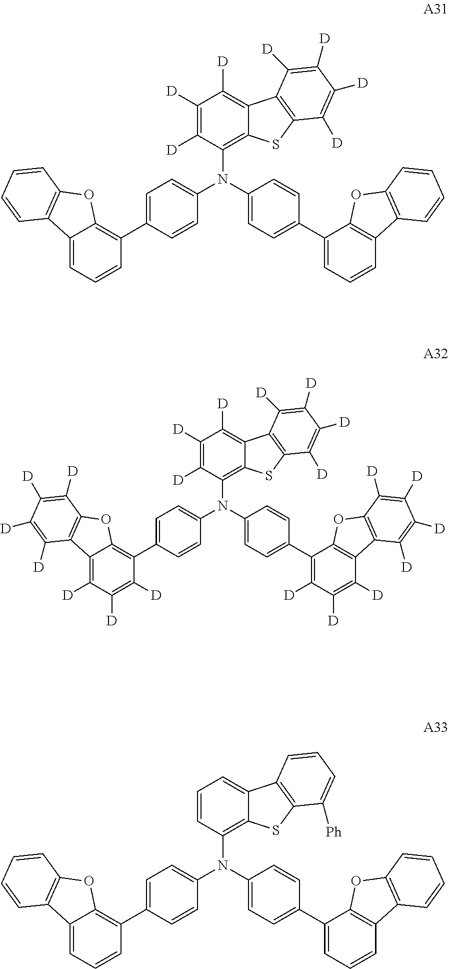 [SMILES: [2H]c1c([2H])c([2H])c2c(sc3c(N(c4ccc(-c5cccc6c5oc5ccccc56)cc4)c4ccc(-c5cccc6c5oc5ccccc56)cc4)c([2H])c([2H])c([2H])c32)c1[2H] ; [2H]c1c([2H])c([2H])c2c(oc3c(-c4ccc(N(c5ccc(-c6c([2H])c([2H])c([2H])c7c6oc6c([2H])c([2H])c([2H])c([2H])c67)cc5)c5c([2H])c([2H])c([2H])c6c5sc5c([2H])c([2H])c([2H])c([2H])c56)cc4)c([2H])c([2H])c([2H])c32)c1[2H] ; c1ccc(-c2cccc3c2sc2c(N(c4ccc(-c5cccc6c5oc5ccccc56)cc4)c4ccc(-c5cccc6c5oc5ccccc56)cc4)cccc23)cc1]

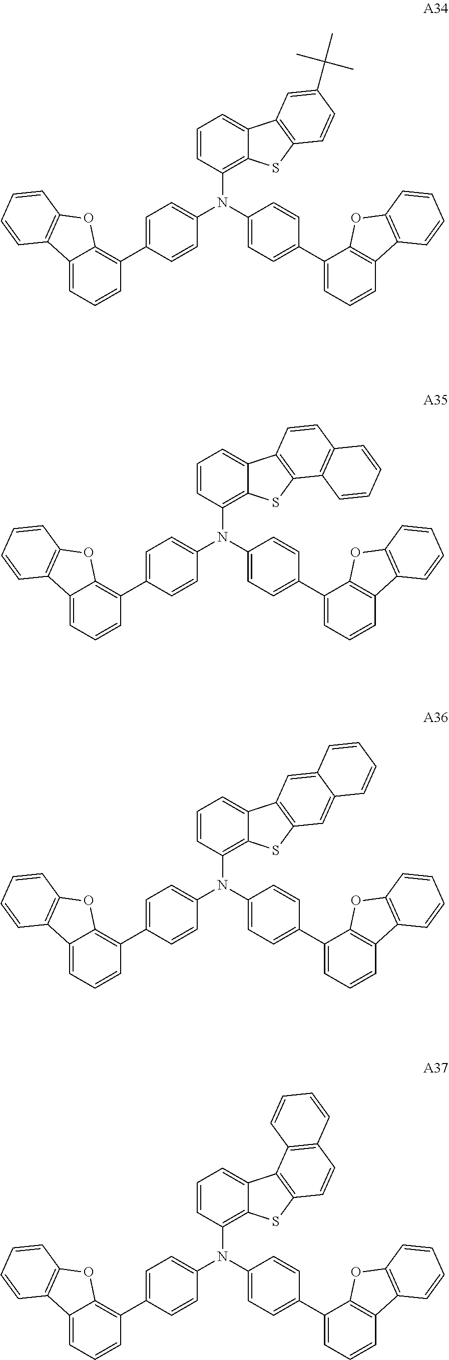 [SMILES: CC(C)(C)c1ccc2sc3c(N(c4ccc(-c5cccc6c5oc5ccccc56)cc4)c4ccc(-c5cccc6c5oc5ccccc56)cc4)cccc3c2c1 ; c1ccc2c(c1)ccc1c3cccc(N(c4ccc(-c5cccc6c5oc5ccccc56)cc4)c4ccc(-c5cccc6c5oc5ccccc56)cc4)c3sc21 ; c1ccc2cc3c(cc2c1)sc1c(N(c2ccc(-c4cccc5c4oc4ccccc45)cc2)c2ccc(-c4cccc5c4oc4ccccc45)cc2)cccc13 ; c1ccc2c(c1)ccc1sc3c(N(c4ccc(-c5cccc6c5oc5ccccc56)cc4)c4ccc(-c5cccc6c5oc5ccccc56)cc4)cccc3c12]

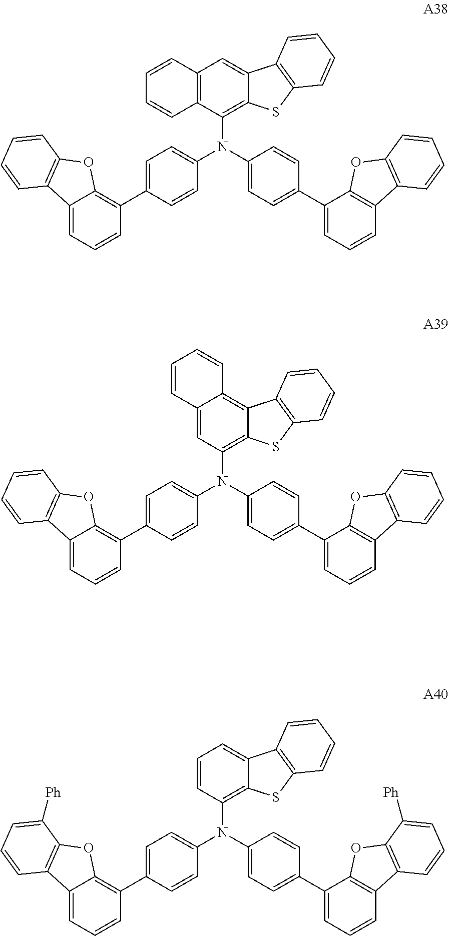 [SMILES: c1ccc2c(N(c3ccc(-c4cccc5c4oc4ccccc45)cc3)c3ccc(-c4cccc5c4oc4ccccc45)cc3)c3sc4ccccc4c3cc2c1 ; c1ccc2c(c1)cc(N(c1ccc(-c3cccc4c3oc3ccccc34)cc1)c1ccc(-c3cccc4c3oc3ccccc34)cc1)c1sc3ccccc3c12 ; c1ccc(-c2cccc3c2oc2c(-c4ccc(N(c5ccc(-c6cccc7c6oc6c(-c8ccccc8)cccc67)cc5)c5cccc6c5sc5ccccc56)cc4)cccc23)cc1]

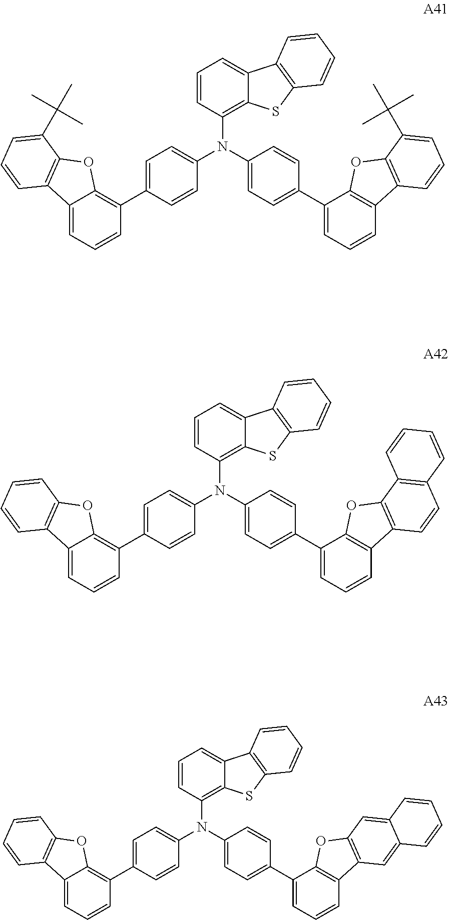 [SMILES: CC(C)(C)c1cccc2c1oc1c(-c3ccc(N(c4ccc(-c5cccc6c5oc5c(C(C)(C)C)cccc56)cc4)c4cccc5c4sc4ccccc45)cc3)cccc12 ; c1ccc2c(c1)ccc1c3cccc(-c4ccc(N(c5ccc(-c6cccc7c6oc6ccccc67)cc5)c5cccc6c5sc5ccccc56)cc4)c3oc21 ; c1ccc2cc3c(cc2c1)oc1c(-c2ccc(N(c4ccc(-c5cccc6c5oc5ccccc56)cc4)c4cccc5c4sc4ccccc45)cc2)cccc13]

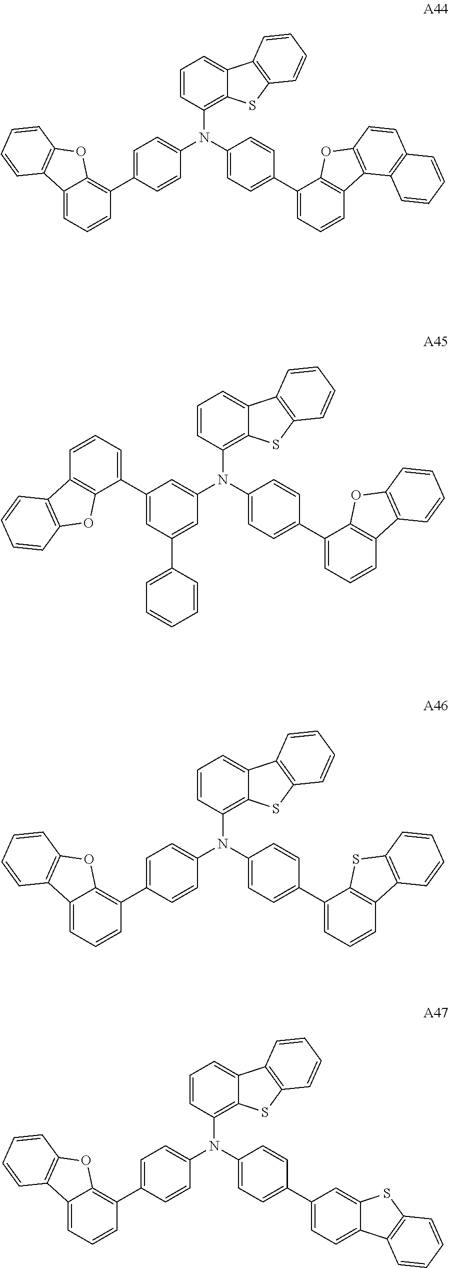 [SMILES: c1ccc2c(c1)ccc1oc3c(-c4ccc(N(c5ccc(-c6cccc7c6oc6ccccc67)cc5)c5cccc6c5sc5ccccc56)cc4)cccc3c12 ; c1ccc(-c2cc(-c3cccc4c3oc3ccccc34)cc(N(c3ccc(-c4cccc5c4oc4ccccc45)cc3)c3cccc4c3sc3ccccc34)c2)cc1 ; c1ccc2c(c1)oc1c(-c3ccc(N(c4ccc(-c5cccc6c5sc5ccccc56)cc4)c4cccc5c4sc4ccccc45)cc3)cccc12 ; c1ccc2c(c1)oc1c(-c3ccc(N(c4ccc(-c5ccc6c(c5)sc5ccccc56)cc4)c4cccc5c4sc4ccccc45)cc3)cccc12]

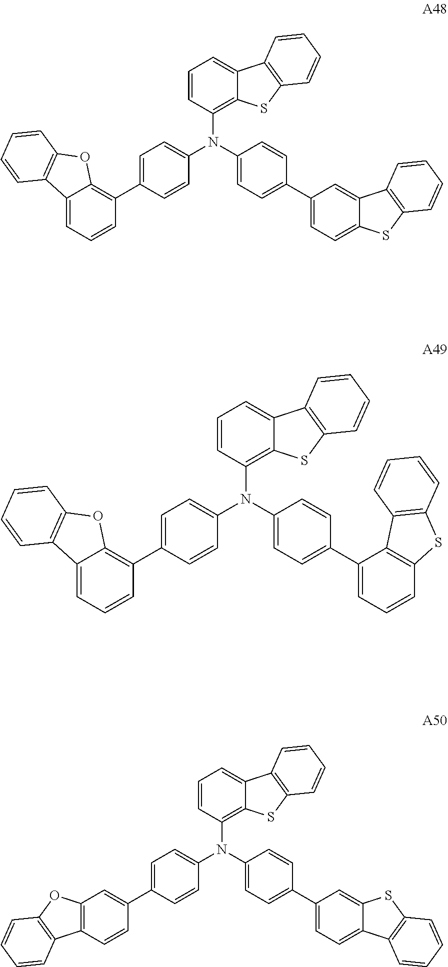 [SMILES: c1ccc2c(c1)oc1c(-c3ccc(N(c4ccc(-c5ccc6sc7ccccc7c6c5)cc4)c4cccc5c4sc4ccccc45)cc3)cccc12 ; c1ccc2c(c1)oc1c(-c3ccc(N(c4ccc(-c5cccc6sc7ccccc7c56)cc4)c4cccc5c4sc4ccccc45)cc3)cccc12 ; c1ccc2c(c1)oc1cc(-c3ccc(N(c4ccc(-c5ccc6c(c5)sc5ccccc56)cc4)c4cccc5c4sc4ccccc45)cc3)ccc12]

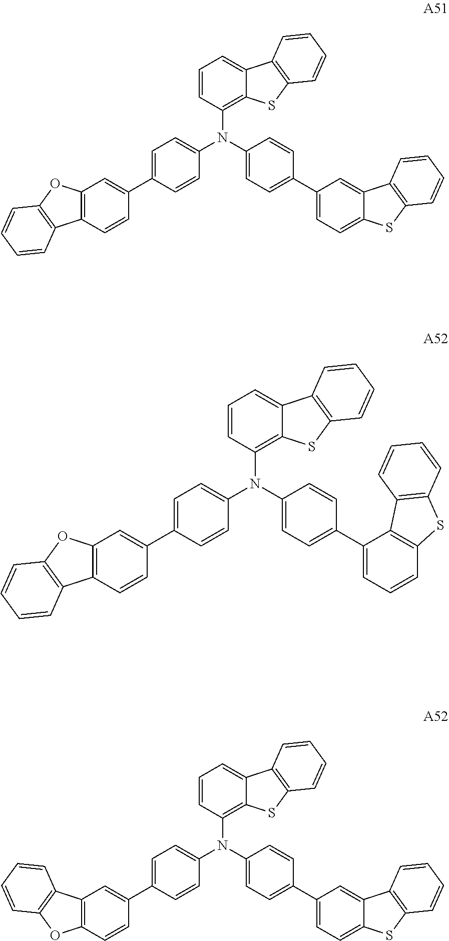 [SMILES: c1ccc2c(c1)oc1cc(-c3ccc(N(c4ccc(-c5ccc6sc7ccccc7c6c5)cc4)c4cccc5c4sc4ccccc45)cc3)ccc12 ; c1ccc2c(c1)oc1cc(-c3ccc(N(c4ccc(-c5cccc6sc7ccccc7c56)cc4)c4cccc5c4sc4ccccc45)cc3)ccc12 ; c1ccc2c(c1)oc1ccc(-c3ccc(N(c4ccc(-c5ccc6sc7ccccc7c6c5)cc4)c4cccc5c4sc4ccccc45)cc3)cc12]

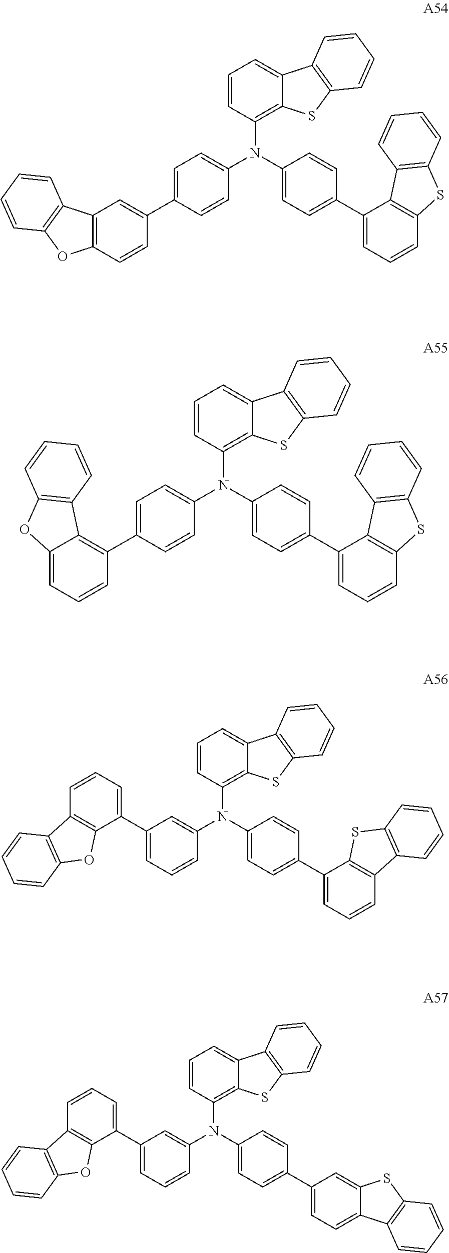 [SMILES: c1ccc2c(c1)oc1ccc(-c3ccc(N(c4ccc(-c5cccc6sc7ccccc7c56)cc4)c4cccc5c4sc4ccccc45)cc3)cc12 ; c1ccc2c(c1)oc1cccc(-c3ccc(N(c4ccc(-c5cccc6sc7ccccc7c56)cc4)c4cccc5c4sc4ccccc45)cc3)c12 ; c1cc(-c2cccc3c2oc2ccccc23)cc(N(c2ccc(-c3cccc4c3sc3ccccc34)cc2)c2cccc3c2sc2ccccc23)c1 ; c1cc(-c2cccc3c2oc2ccccc23)cc(N(c2ccc(-c3ccc4c(c3)sc3ccccc34)cc2)c2cccc3c2sc2ccccc23)c1]

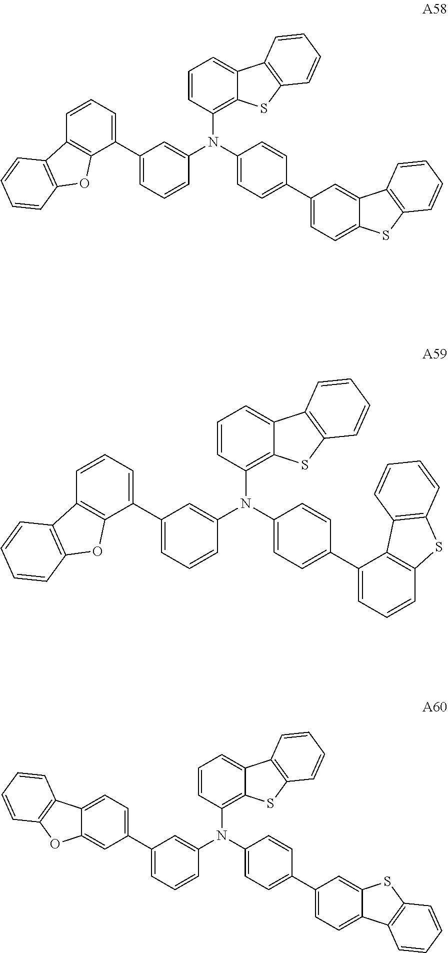 [SMILES: c1cc(-c2cccc3c2oc2ccccc23)cc(N(c2ccc(-c3ccc4sc5ccccc5c4c3)cc2)c2cccc3c2sc2ccccc23)c1 ; c1cc(-c2cccc3c2oc2ccccc23)cc(N(c2ccc(-c3cccc4sc5ccccc5c34)cc2)c2cccc3c2sc2ccccc23)c1 ; c1cc(-c2ccc3c(c2)oc2ccccc23)cc(N(c2ccc(-c3ccc4c(c3)sc3ccccc34)cc2)c2cccc3c2sc2ccccc23)c1]

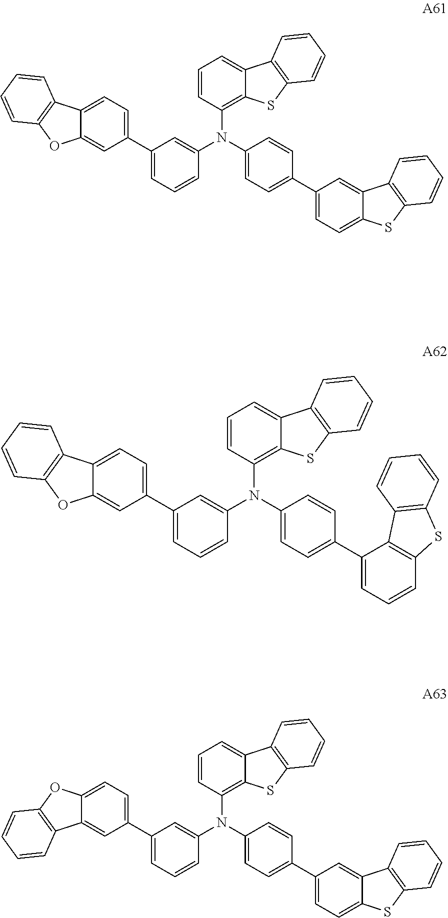 [SMILES: c1cc(-c2ccc3c(c2)oc2ccccc23)cc(N(c2ccc(-c3ccc4sc5ccccc5c4c3)cc2)c2cccc3c2sc2ccccc23)c1 ; c1cc(-c2ccc3c(c2)oc2ccccc23)cc(N(c2ccc(-c3cccc4sc5ccccc5c34)cc2)c2cccc3c2sc2ccccc23)c1 ; c1cc(-c2ccc3oc4ccccc4c3c2)cc(N(c2ccc(-c3ccc4sc5ccccc5c4c3)cc2)c2cccc3c2sc2ccccc23)c1]

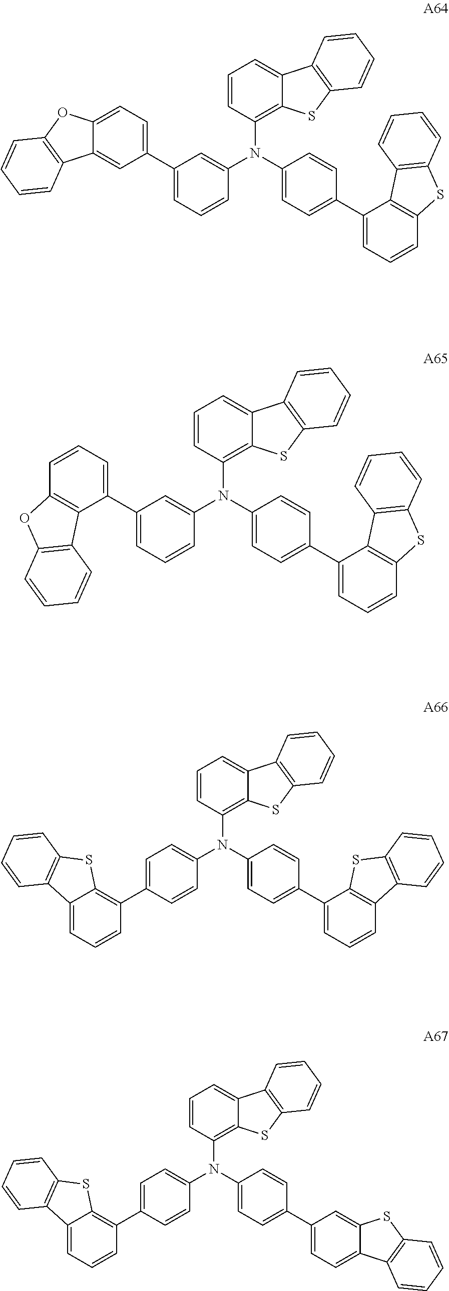 [SMILES: c1cc(-c2ccc3oc4ccccc4c3c2)cc(N(c2ccc(-c3cccc4sc5ccccc5c34)cc2)c2cccc3c2sc2ccccc23)c1 ; c1cc(-c2cccc3oc4ccccc4c23)cc(N(c2ccc(-c3cccc4sc5ccccc5c34)cc2)c2cccc3c2sc2ccccc23)c1 ; c1ccc2c(c1)sc1c(-c3ccc(N(c4ccc(-c5cccc6c5sc5ccccc56)cc4)c4cccc5c4sc4ccccc45)cc3)cccc12 ; c1ccc2c(c1)sc1cc(-c3ccc(N(c4ccc(-c5cccc6c5sc5ccccc56)cc4)c4cccc5c4sc4ccccc45)cc3)ccc12]

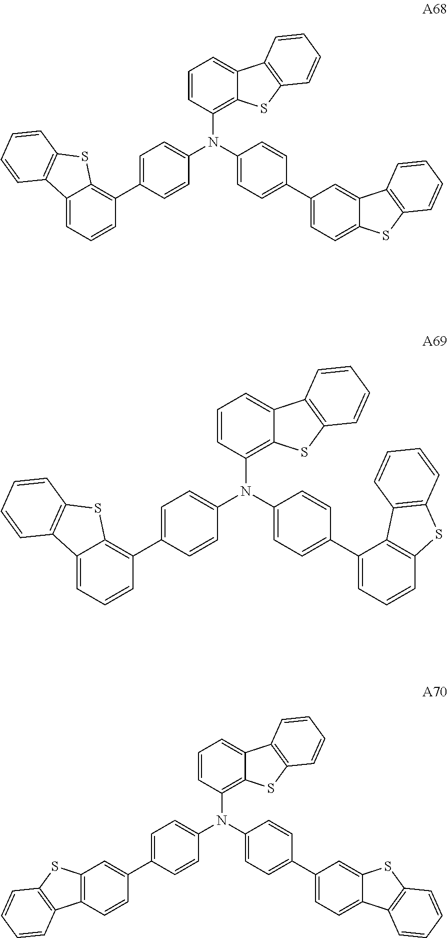 [SMILES: c1ccc2c(c1)sc1ccc(-c3ccc(N(c4ccc(-c5cccc6c5sc5ccccc56)cc4)c4cccc5c4sc4ccccc45)cc3)cc12 ; c1ccc2c(c1)sc1c(-c3ccc(N(c4ccc(-c5cccc6sc7ccccc7c56)cc4)c4cccc5c4sc4ccccc45)cc3)cccc12 ; c1ccc2c(c1)sc1cc(-c3ccc(N(c4ccc(-c5ccc6c(c5)sc5ccccc56)cc4)c4cccc5c4sc4ccccc45)cc3)ccc12]

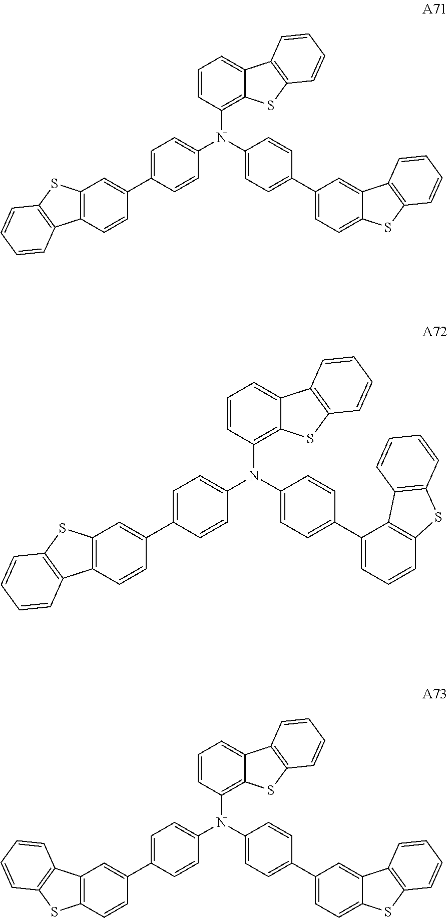 [SMILES: c1ccc2c(c1)sc1cc(-c3ccc(N(c4ccc(-c5ccc6sc7ccccc7c6c5)cc4)c4cccc5c4sc4ccccc45)cc3)ccc12 ; c1ccc2c(c1)sc1cc(-c3ccc(N(c4ccc(-c5cccc6sc7ccccc7c56)cc4)c4cccc5c4sc4ccccc45)cc3)ccc12 ; c1ccc2c(c1)sc1ccc(-c3ccc(N(c4ccc(-c5ccc6sc7ccccc7c6c5)cc4)c4cccc5c4sc4ccccc45)cc3)cc12]

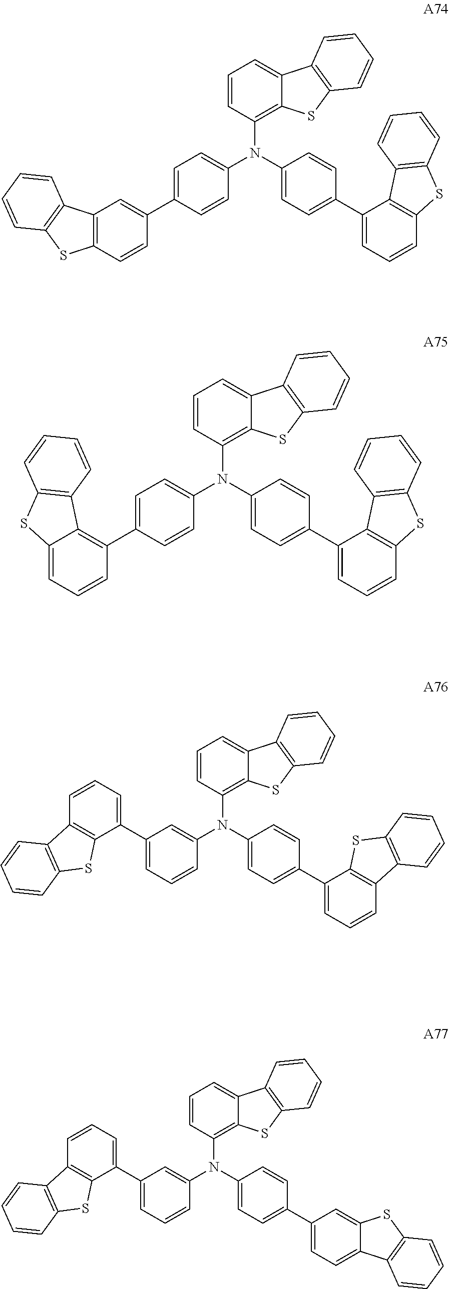 [SMILES: c1ccc2c(c1)sc1ccc(-c3ccc(N(c4ccc(-c5cccc6sc7ccccc7c56)cc4)c4cccc5c4sc4ccccc45)cc3)cc12 ; c1ccc2c(c1)sc1c(N(c3ccc(-c4cccc5sc6ccccc6c45)cc3)c3ccc(-c4cccc5sc6ccccc6c45)cc3)cccc12 ; c1cc(-c2cccc3c2sc2ccccc23)cc(N(c2ccc(-c3cccc4c3sc3ccccc34)cc2)c2cccc3c2sc2ccccc23)c1 ; c1cc(-c2cccc3c2sc2ccccc23)cc(N(c2ccc(-c3ccc4c(c3)sc3ccccc34)cc2)c2cccc3c2sc2ccccc23)c1]

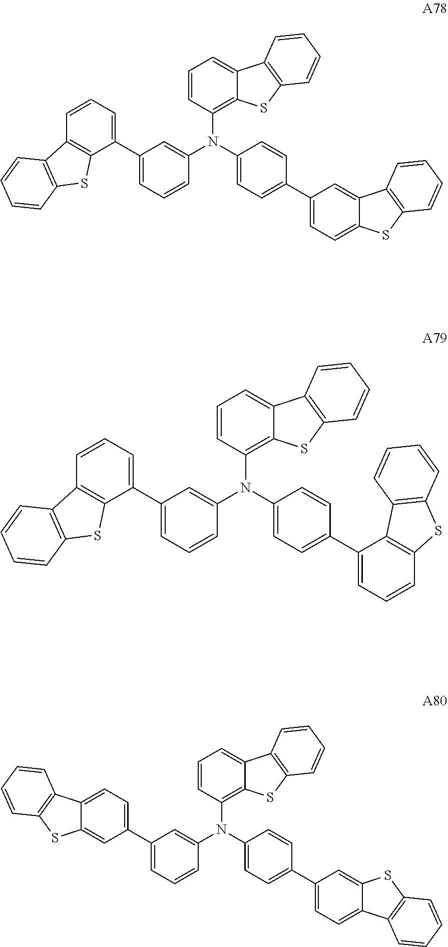 [SMILES: c1cc(-c2cccc3c2sc2ccccc23)cc(N(c2ccc(-c3ccc4sc5ccccc5c4c3)cc2)c2cccc3c2sc2ccccc23)c1 ; c1cc(-c2cccc3c2sc2ccccc23)cc(N(c2ccc(-c3cccc4sc5ccccc5c34)cc2)c2cccc3c2sc2ccccc23)c1 ; c1cc(-c2ccc3c(c2)sc2ccccc23)cc(N(c2ccc(-c3ccc4c(c3)sc3ccccc34)cc2)c2cccc3c2sc2ccccc23)c1]

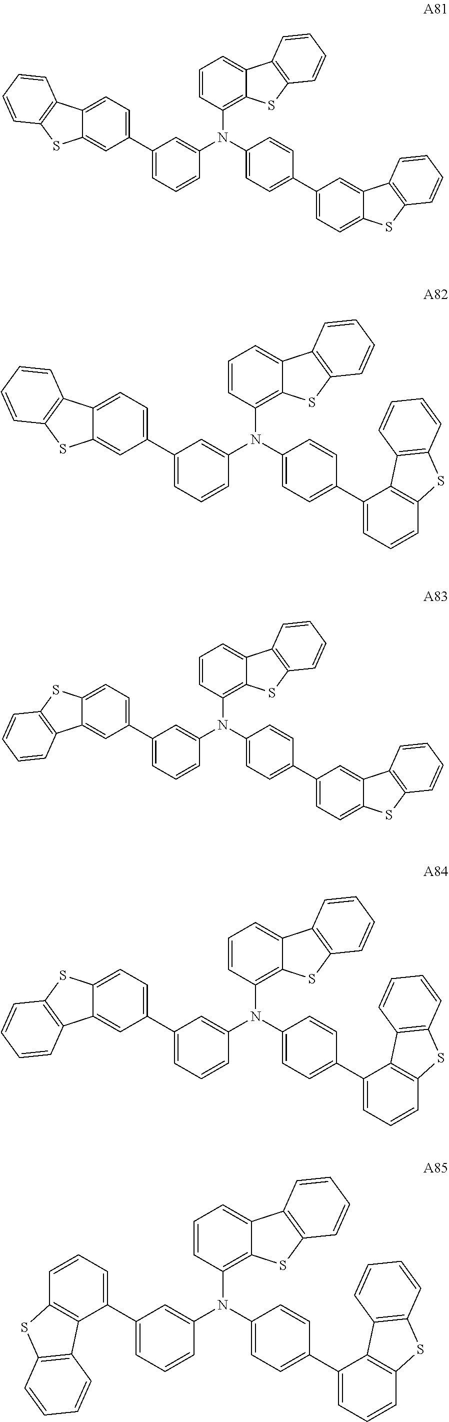 [SMILES: c1cc(-c2ccc3c(c2)sc2ccccc23)cc(N(c2ccc(-c3ccc4sc5ccccc5c4c3)cc2)c2cccc3c2sc2ccccc23)c1 ; c1cc(-c2ccc3c(c2)sc2ccccc23)cc(N(c2ccc(-c3cccc4sc5ccccc5c34)cc2)c2cccc3c2sc2ccccc23)c1 ; c1cc(-c2ccc3sc4ccccc4c3c2)cc(N(c2ccc(-c3ccc4sc5ccccc5c4c3)cc2)c2cccc3c2sc2ccccc23)c1 ; c1cc(-c2ccc3sc4ccccc4c3c2)cc(N(c2ccc(-c3cccc4sc5ccccc5c34)cc2)c2cccc3c2sc2ccccc23)c1 ; c1cc(-c2cccc3sc4ccccc4c23)cc(N(c2ccc(-c3cccc4sc5ccccc5c34)cc2)c2cccc3c2sc2ccccc23)c1]

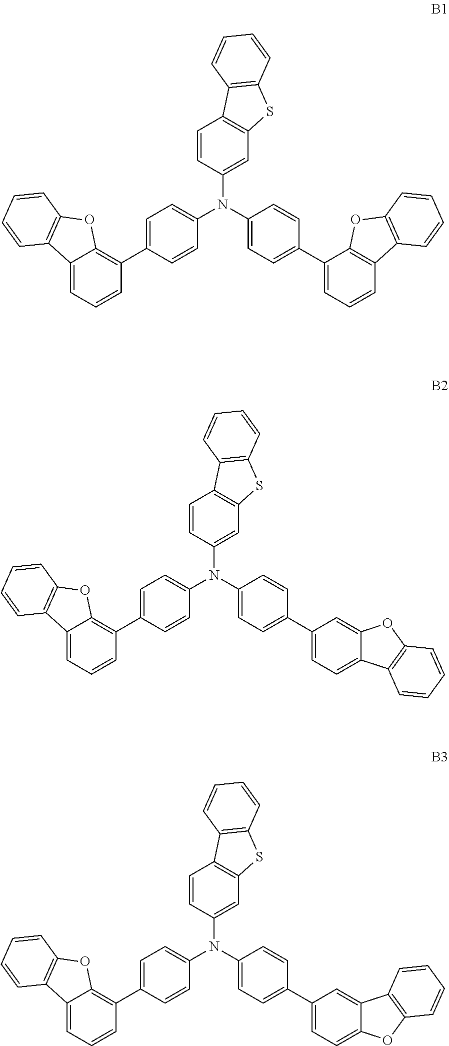 [SMILES: c1ccc2c(c1)oc1c(-c3ccc(N(c4ccc(-c5cccc6c5oc5ccccc56)cc4)c4ccc5c(c4)sc4ccccc45)cc3)cccc12 ; c1ccc2c(c1)oc1cc(-c3ccc(N(c4ccc(-c5cccc6c5oc5ccccc56)cc4)c4ccc5c(c4)sc4ccccc45)cc3)ccc12 ; c1ccc2c(c1)oc1ccc(-c3ccc(N(c4ccc(-c5cccc6c5oc5ccccc56)cc4)c4ccc5c(c4)sc4ccccc45)cc3)cc12]

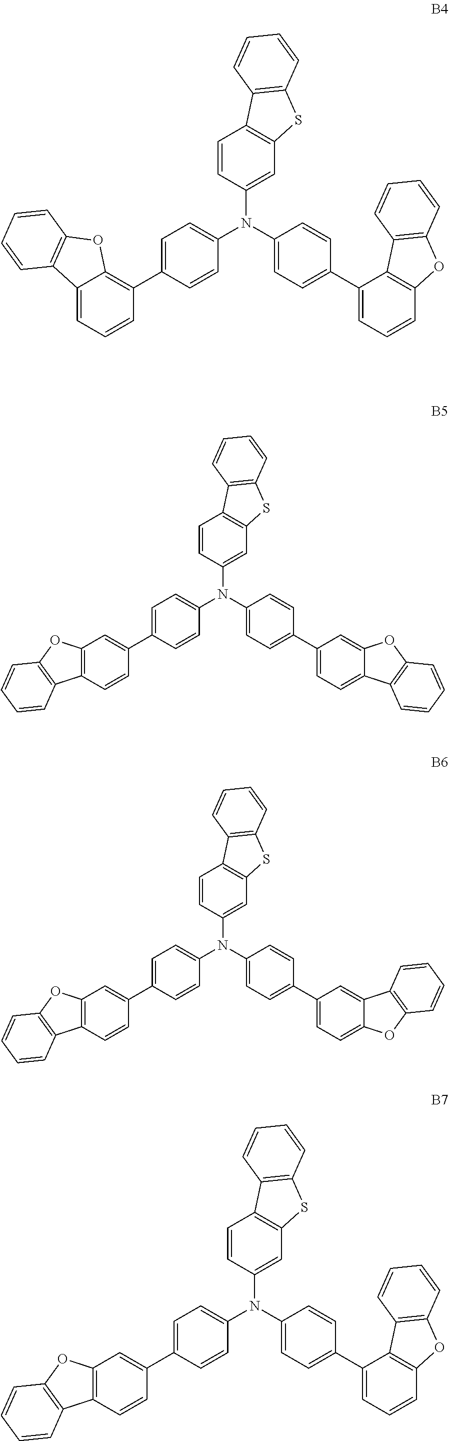 [SMILES: c1ccc2c(c1)oc1c(-c3ccc(N(c4ccc(-c5cccc6oc7ccccc7c56)cc4)c4ccc5c(c4)sc4ccccc45)cc3)cccc12 ; c1ccc2c(c1)oc1cc(-c3ccc(N(c4ccc(-c5ccc6c(c5)oc5ccccc56)cc4)c4ccc5c(c4)sc4ccccc45)cc3)ccc12 ; c1ccc2c(c1)oc1cc(-c3ccc(N(c4ccc(-c5ccc6oc7ccccc7c6c5)cc4)c4ccc5c(c4)sc4ccccc45)cc3)ccc12 ; c1ccc2c(c1)oc1cc(-c3ccc(N(c4ccc(-c5cccc6oc7ccccc7c56)cc4)c4ccc5c(c4)sc4ccccc45)cc3)ccc12]

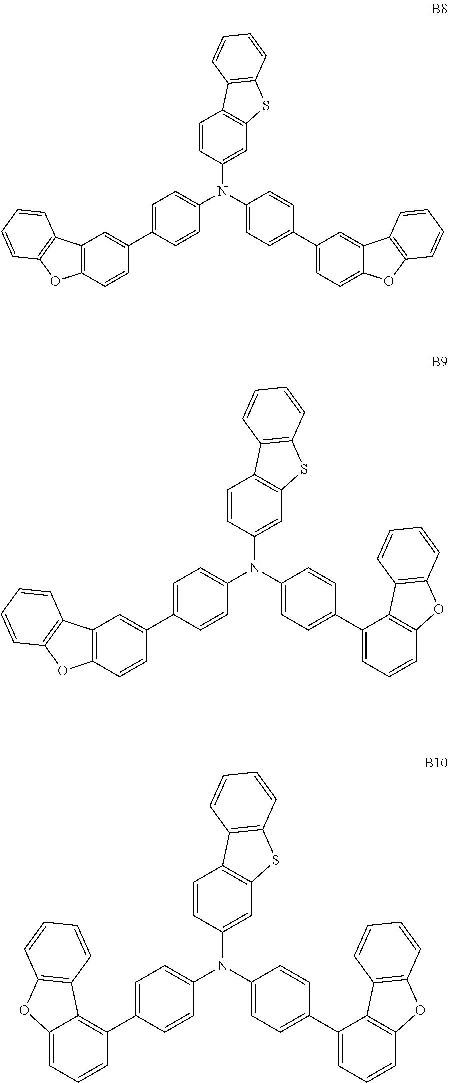 [SMILES: c1ccc2c(c1)oc1ccc(-c3ccc(N(c4ccc(-c5ccc6oc7ccccc7c6c5)cc4)c4ccc5c(c4)sc4ccccc45)cc3)cc12 ; c1ccc2c(c1)oc1ccc(-c3ccc(N(c4ccc(-c5cccc6oc7ccccc7c56)cc4)c4ccc5c(c4)sc4ccccc45)cc3)cc12 ; c1ccc2c(c1)oc1cccc(-c3ccc(N(c4ccc(-c5cccc6oc7ccccc7c56)cc4)c4ccc5c(c4)sc4ccccc45)cc3)c12]

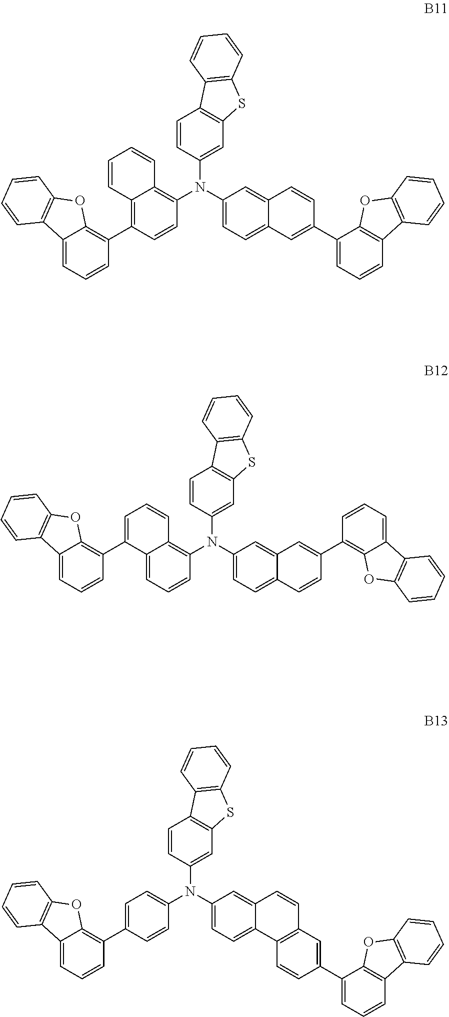 [SMILES: c1ccc2c(c1)oc1c(-c3ccc4cc(N(c5ccc6c(c5)sc5ccccc56)c5ccc(-c6cccc7c6oc6ccccc67)c6ccccc56)ccc4c3)cccc12 ; c1ccc2c(c1)oc1c(-c3ccc4ccc(N(c5ccc6c(c5)sc5ccccc56)c5cccc6c(-c7cccc8c7oc7ccccc78)cccc56)cc4c3)cccc12 ; c1ccc2c(c1)oc1c(-c3ccc(N(c4ccc5c(ccc6cc(-c7cccc8c7oc7ccccc78)ccc65)c4)c4ccc5c(c4)sc4ccccc45)cc3)cccc12]

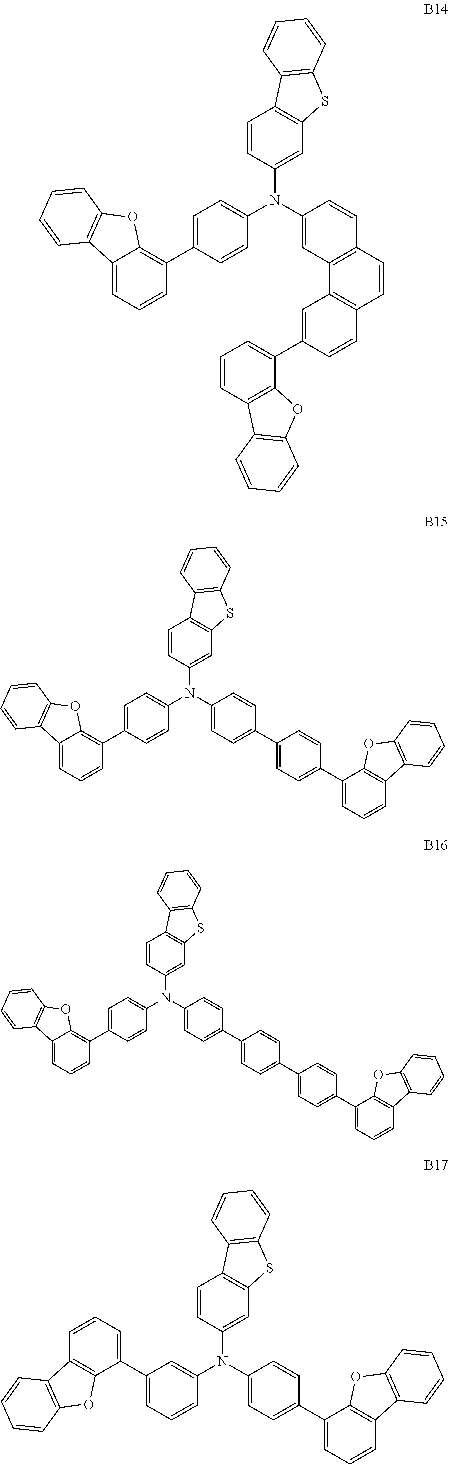 [SMILES: c1ccc2c(c1)oc1c(-c3ccc(N(c4ccc5c(c4)sc4ccccc45)c4ccc5ccc6ccc(-c7cccc8c7oc7ccccc78)cc6c5c4)cc3)cccc12 ; c1ccc2c(c1)oc1c(-c3ccc(-c4ccc(N(c5ccc(-c6cccc7c6oc6ccccc67)cc5)c5ccc6c(c5)sc5ccccc56)cc4)cc3)cccc12 ; c1ccc2c(c1)oc1c(-c3ccc(-c4ccc(-c5ccc(N(c6ccc(-c7cccc8c7oc7ccccc78)cc6)c6ccc7c(c6)sc6ccccc67)cc5)cc4)cc3)cccc12 ; c1cc(-c2cccc3c2oc2ccccc23)cc(N(c2ccc(-c3cccc4c3oc3ccccc34)cc2)c2ccc3c(c2)sc2ccccc23)c1]

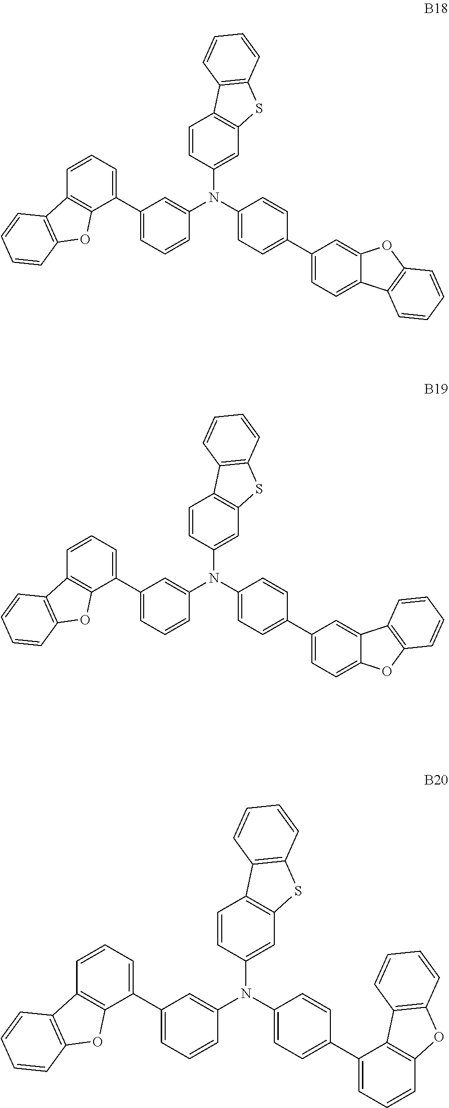 [SMILES: c1cc(-c2cccc3c2oc2ccccc23)cc(N(c2ccc(-c3ccc4c(c3)oc3ccccc34)cc2)c2ccc3c(c2)sc2ccccc23)c1 ; c1cc(-c2cccc3c2oc2ccccc23)cc(N(c2ccc(-c3ccc4oc5ccccc5c4c3)cc2)c2ccc3c(c2)sc2ccccc23)c1 ; c1cc(-c2cccc3c2oc2ccccc23)cc(N(c2ccc(-c3cccc4oc5ccccc5c34)cc2)c2ccc3c(c2)sc2ccccc23)c1]

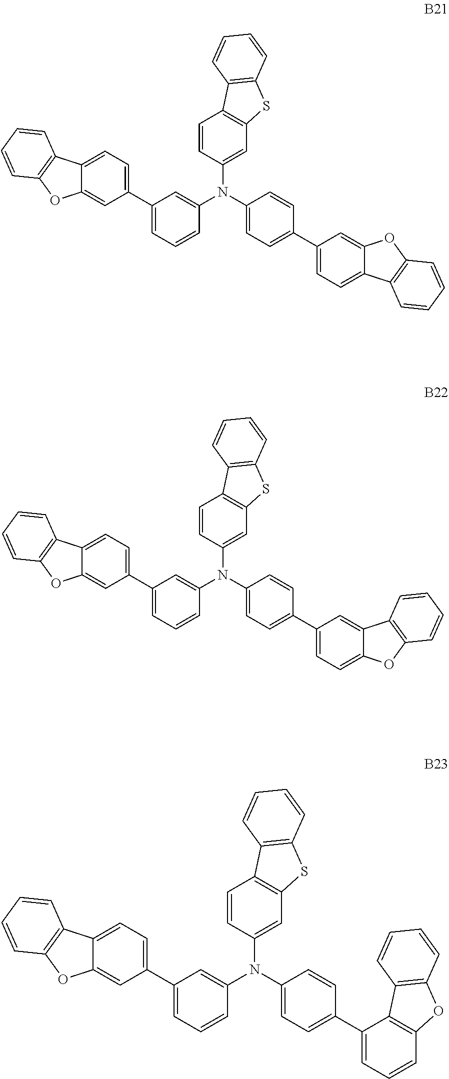 [SMILES: c1cc(-c2ccc3c(c2)oc2ccccc23)cc(N(c2ccc(-c3ccc4c(c3)oc3ccccc34)cc2)c2ccc3c(c2)sc2ccccc23)c1 ; c1cc(-c2ccc3c(c2)oc2ccccc23)cc(N(c2ccc(-c3ccc4oc5ccccc5c4c3)cc2)c2ccc3c(c2)sc2ccccc23)c1 ; c1cc(-c2ccc3c(c2)oc2ccccc23)cc(N(c2ccc(-c3cccc4oc5ccccc5c34)cc2)c2ccc3c(c2)sc2ccccc23)c1]

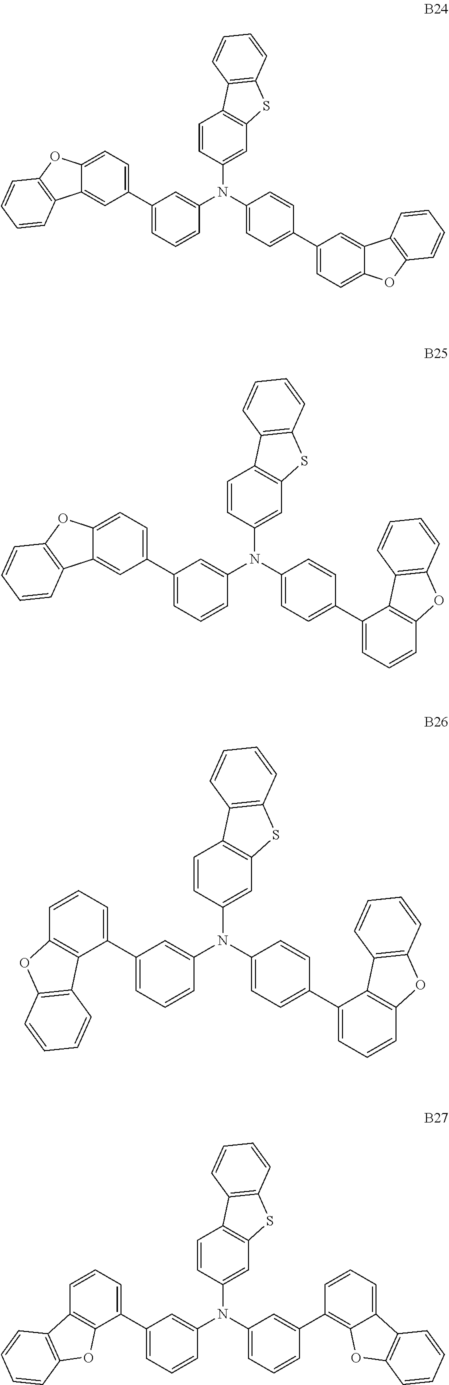 [SMILES: c1cc(-c2ccc3oc4ccccc4c3c2)cc(N(c2ccc(-c3ccc4oc5ccccc5c4c3)cc2)c2ccc3c(c2)sc2ccccc23)c1 ; c1cc(-c2ccc3oc4ccccc4c3c2)cc(N(c2ccc(-c3cccc4oc5ccccc5c34)cc2)c2ccc3c(c2)sc2ccccc23)c1 ; c1cc(-c2cccc3oc4ccccc4c23)cc(N(c2ccc(-c3cccc4oc5ccccc5c34)cc2)c2ccc3c(c2)sc2ccccc23)c1 ; c1cc(-c2cccc3c2oc2ccccc23)cc(N(c2cccc(-c3cccc4c3oc3ccccc34)c2)c2ccc3c(c2)sc2ccccc23)c1]

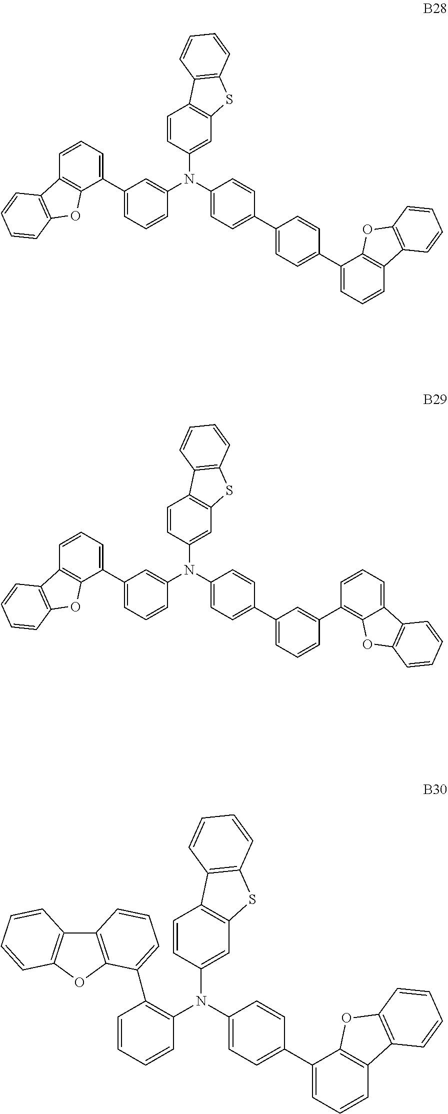 [SMILES: c1cc(-c2cccc3c2oc2ccccc23)cc(N(c2ccc(-c3ccc(-c4cccc5c4oc4ccccc45)cc3)cc2)c2ccc3c(c2)sc2ccccc23)c1 ; c1cc(-c2ccc(N(c3cccc(-c4cccc5c4oc4ccccc45)c3)c3ccc4c(c3)sc3ccccc34)cc2)cc(-c2cccc3c2oc2ccccc23)c1 ; c1ccc(N(c2ccc(-c3cccc4c3oc3ccccc34)cc2)c2ccc3c(c2)sc2ccccc23)c(-c2cccc3c2oc2ccccc23)c1]

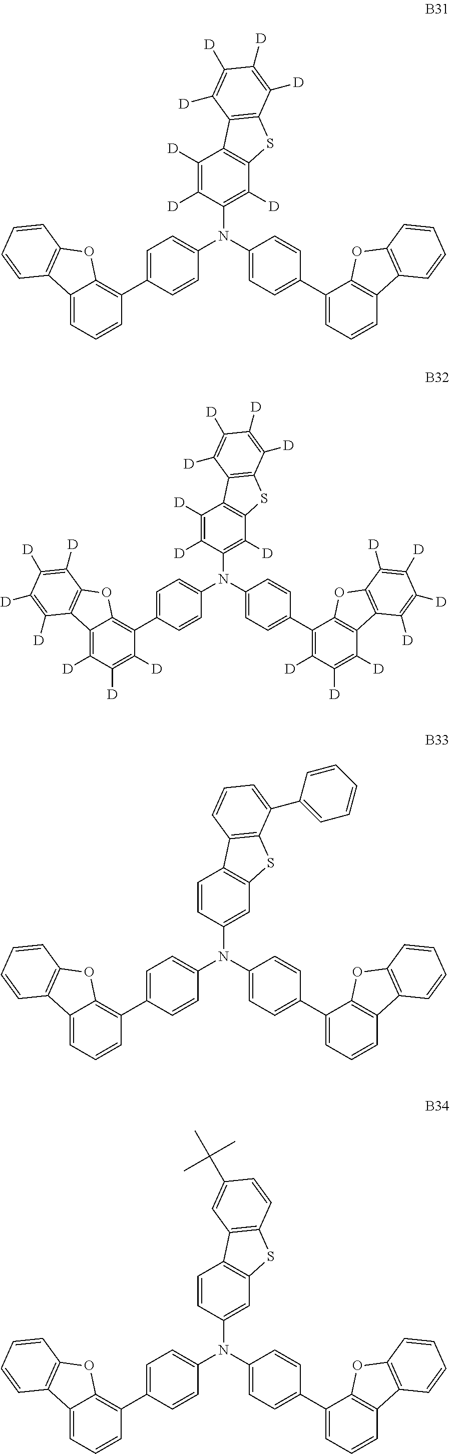

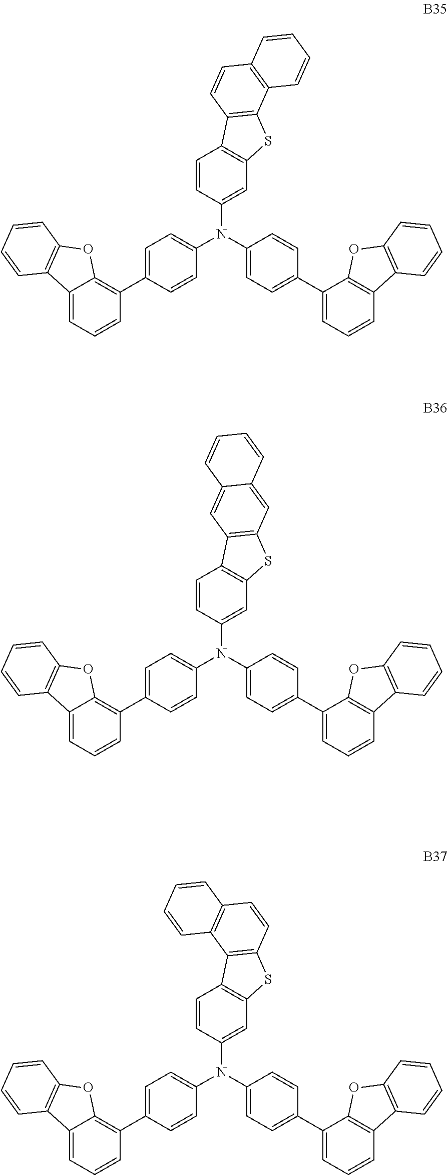 [SMILES: c1ccc2c(c1)ccc1c3ccc(N(c4ccc(-c5cccc6c5oc5ccccc56)cc4)c4ccc(-c5cccc6c5oc5ccccc56)cc4)cc3sc21 ; c1ccc2cc3c(cc2c1)sc1cc(N(c2ccc(-c4cccc5c4oc4ccccc45)cc2)c2ccc(-c4cccc5c4oc4ccccc45)cc2)ccc13 ; c1ccc2c(c1)ccc1sc3cc(N(c4ccc(-c5cccc6c5oc5ccccc56)cc4)c4ccc(-c5cccc6c5oc5ccccc56)cc4)ccc3c12]

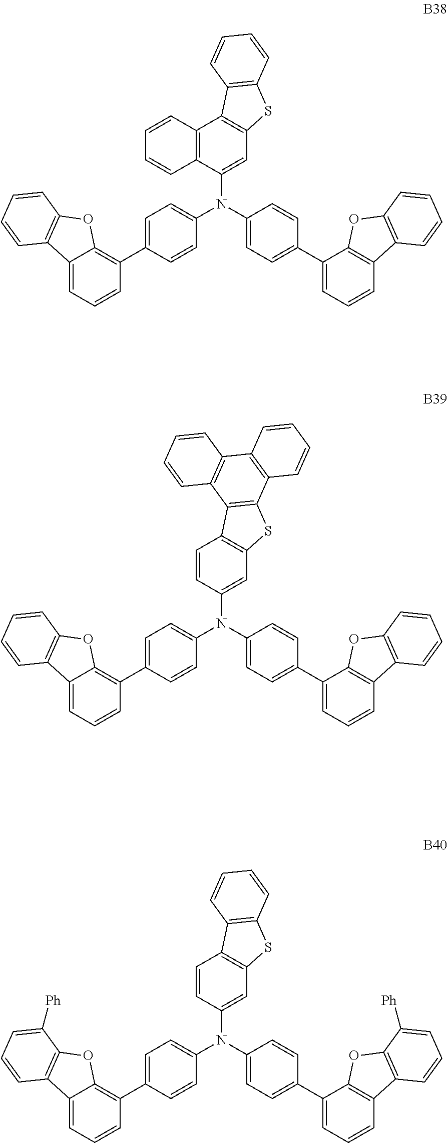 [SMILES: c1ccc2c(c1)oc1c(-c3ccc(N(c4ccc(-c5cccc6c5oc5ccccc56)cc4)c4cc5sc6ccccc6c5c5ccccc45)cc3)cccc12 ; c1ccc2c(c1)oc1c(-c3ccc(N(c4ccc(-c5cccc6c5oc5ccccc56)cc4)c4ccc5c(c4)sc4c6ccccc6c6ccccc6c54)cc3)cccc12 ; c1ccc(-c2cccc3c2oc2c(-c4ccc(N(c5ccc(-c6cccc7c6oc6c(-c8ccccc8)cccc67)cc5)c5ccc6c(c5)sc5ccccc56)cc4)cccc23)cc1]

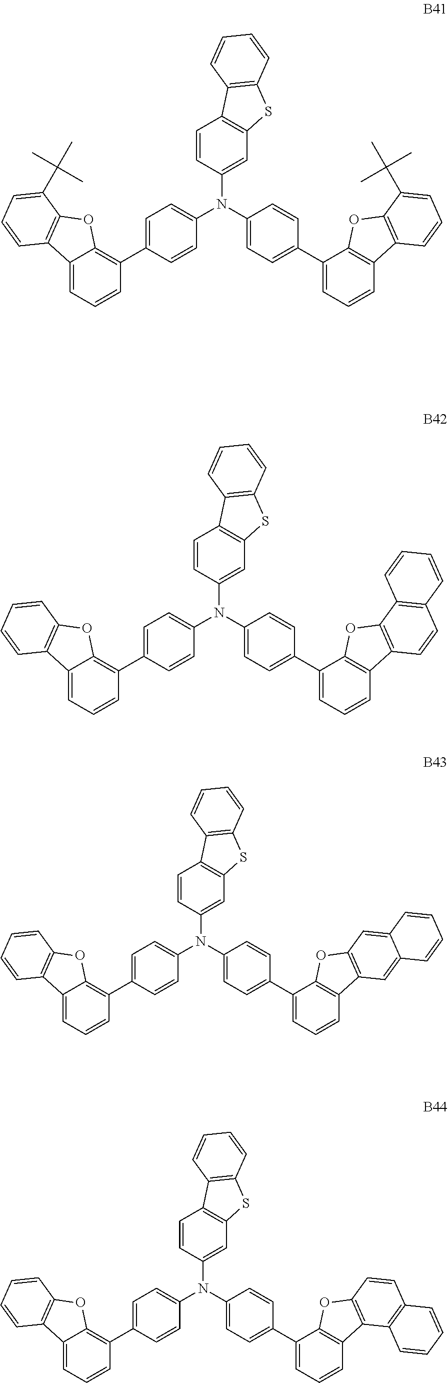 [SMILES: CC(C)(C)c1cccc2c1oc1c(-c3ccc(N(c4ccc(-c5cccc6c5oc5c(C(C)(C)C)cccc56)cc4)c4ccc5c(c4)sc4ccccc45)cc3)cccc12 ; c1ccc2c(c1)ccc1c3cccc(-c4ccc(N(c5ccc(-c6cccc7c6oc6ccccc67)cc5)c5ccc6c(c5)sc5ccccc56)cc4)c3oc21 ; c1ccc2cc3c(cc2c1)oc1c(-c2ccc(N(c4ccc(-c5cccc6c5oc5ccccc56)cc4)c4ccc5c(c4)sc4ccccc45)cc2)cccc13 ; c1ccc2c(c1)ccc1oc3c(-c4ccc(N(c5ccc(-c6cccc7c6oc6ccccc67)cc5)c5ccc6c(c5)sc5ccccc56)cc4)cccc3c12]

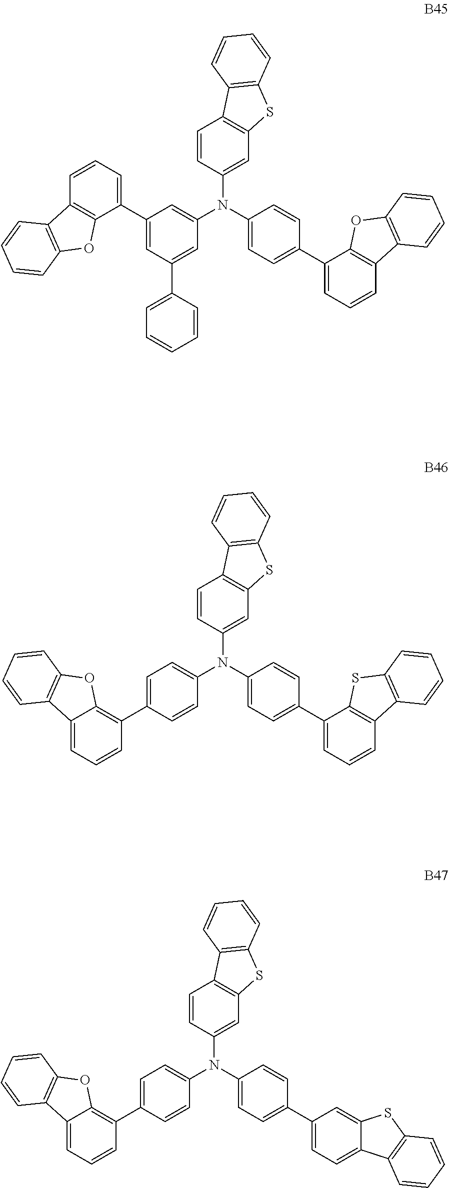 [SMILES: c1ccc(-c2cc(-c3cccc4c3oc3ccccc34)cc(N(c3ccc(-c4cccc5c4oc4ccccc45)cc3)c3ccc4c(c3)sc3ccccc34)c2)cc1 ; c1ccc2c(c1)oc1c(-c3ccc(N(c4ccc(-c5cccc6c5sc5ccccc56)cc4)c4ccc5c(c4)sc4ccccc45)cc3)cccc12 ; c1ccc2c(c1)oc1c(-c3ccc(N(c4ccc(-c5ccc6c(c5)sc5ccccc56)cc4)c4ccc5c(c4)sc4ccccc45)cc3)cccc12]

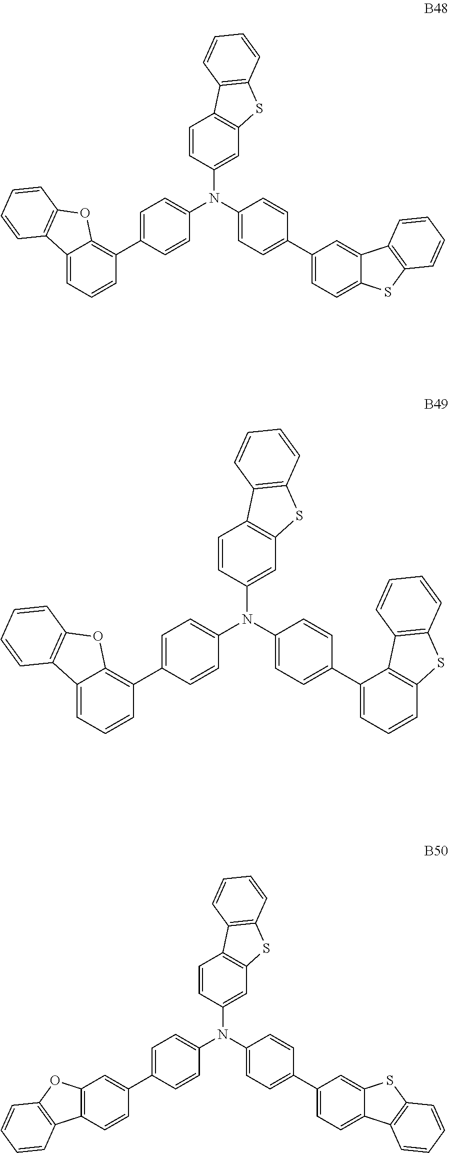 [SMILES: c1ccc2c(c1)oc1c(-c3ccc(N(c4ccc(-c5ccc6sc7ccccc7c6c5)cc4)c4ccc5c(c4)sc4ccccc45)cc3)cccc12 ; c1ccc2c(c1)oc1c(-c3ccc(N(c4ccc(-c5cccc6sc7ccccc7c56)cc4)c4ccc5c(c4)sc4ccccc45)cc3)cccc12 ; c1ccc2c(c1)oc1cc(-c3ccc(N(c4ccc(-c5ccc6c(c5)sc5ccccc56)cc4)c4ccc5c(c4)sc4ccccc45)cc3)ccc12]

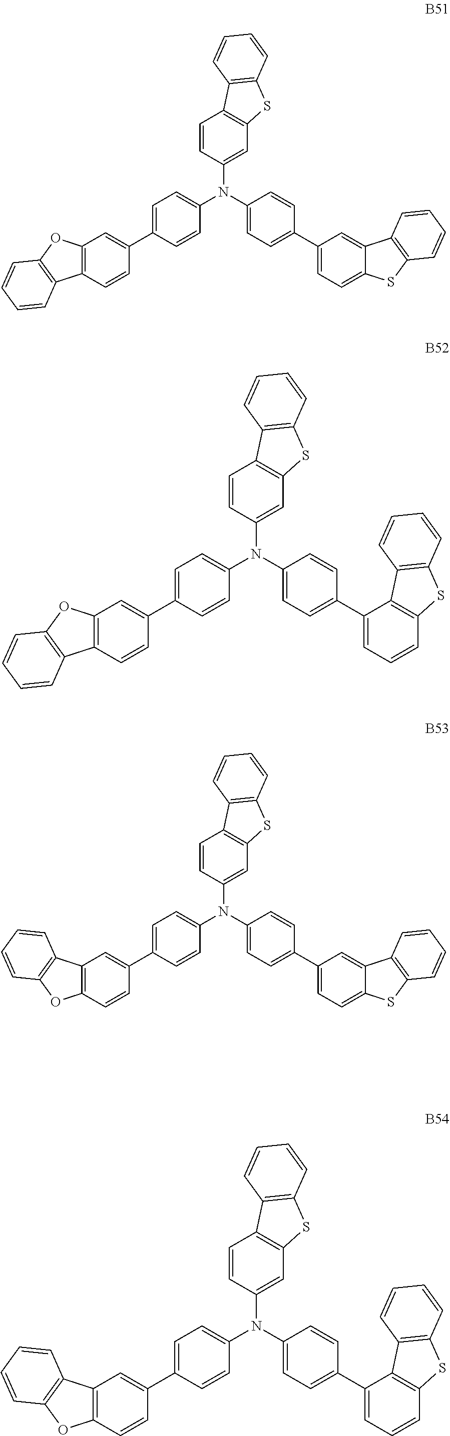 [SMILES: c1ccc2c(c1)oc1cc(-c3ccc(N(c4ccc(-c5ccc6sc7ccccc7c6c5)cc4)c4ccc5c(c4)sc4ccccc45)cc3)ccc12 ; c1ccc2c(c1)oc1cc(-c3ccc(N(c4ccc(-c5cccc6sc7ccccc7c56)cc4)c4ccc5c(c4)sc4ccccc45)cc3)ccc12 ; c1ccc2c(c1)oc1ccc(-c3ccc(N(c4ccc(-c5ccc6sc7ccccc7c6c5)cc4)c4ccc5c(c4)sc4ccccc45)cc3)cc12 ; c1ccc2c(c1)oc1ccc(-c3ccc(N(c4ccc(-c5cccc6sc7ccccc7c56)cc4)c4ccc5c(c4)sc4ccccc45)cc3)cc12]

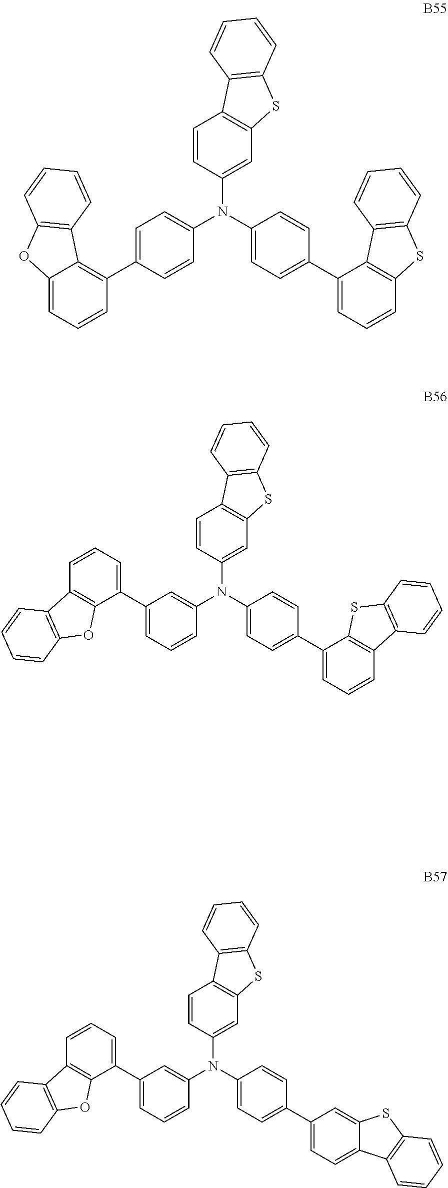 [SMILES: c1ccc2c(c1)oc1cccc(-c3ccc(N(c4ccc(-c5cccc6sc7ccccc7c56)cc4)c4ccc5c(c4)sc4ccccc45)cc3)c12 ; c1cc(-c2cccc3c2oc2ccccc23)cc(N(c2ccc(-c3cccc4c3sc3ccccc34)cc2)c2ccc3c(c2)sc2ccccc23)c1 ; c1cc(-c2cccc3c2oc2ccccc23)cc(N(c2ccc(-c3ccc4c(c3)sc3ccccc34)cc2)c2ccc3c(c2)sc2ccccc23)c1]

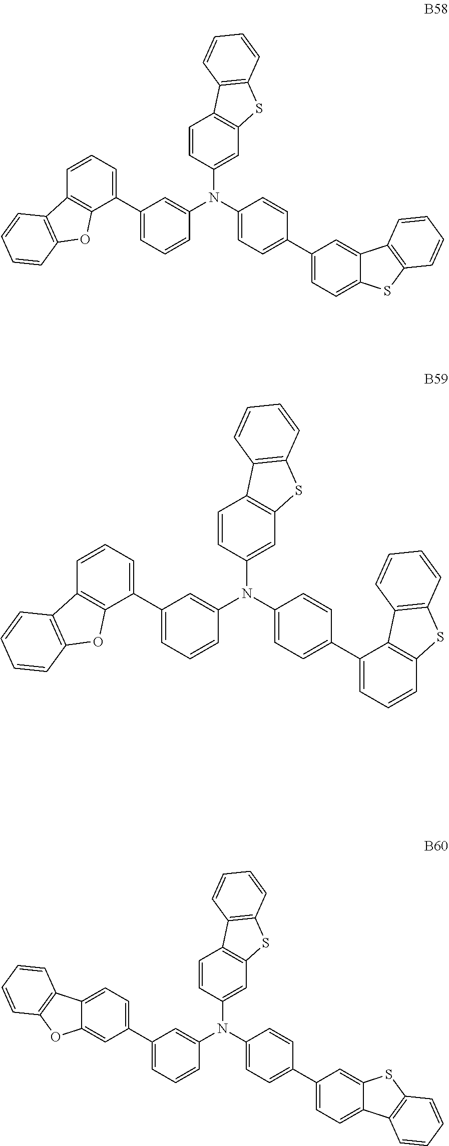 [SMILES: c1cc(-c2cccc3c2oc2ccccc23)cc(N(c2ccc(-c3ccc4sc5ccccc5c4c3)cc2)c2ccc3c(c2)sc2ccccc23)c1 ; c1cc(-c2cccc3c2oc2ccccc23)cc(N(c2ccc(-c3cccc4sc5ccccc5c34)cc2)c2ccc3c(c2)sc2ccccc23)c1 ; c1cc(-c2ccc3c(c2)oc2ccccc23)cc(N(c2ccc(-c3ccc4c(c3)sc3ccccc34)cc2)c2ccc3c(c2)sc2ccccc23)c1]

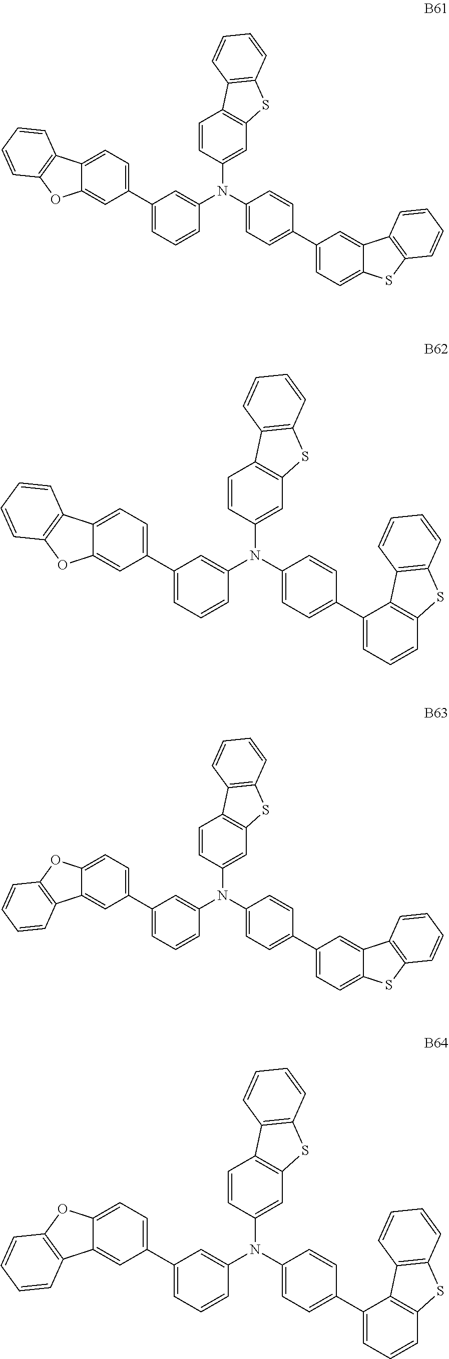 [SMILES: c1cc(-c2ccc3c(c2)oc2ccccc23)cc(N(c2ccc(-c3ccc4sc5ccccc5c4c3)cc2)c2ccc3c(c2)sc2ccccc23)c1 ; c1cc(-c2ccc3c(c2)oc2ccccc23)cc(N(c2ccc(-c3cccc4sc5ccccc5c34)cc2)c2ccc3c(c2)sc2ccccc23)c1 ; c1cc(-c2ccc3oc4ccccc4c3c2)cc(N(c2ccc(-c3ccc4sc5ccccc5c4c3)cc2)c2ccc3c(c2)sc2ccccc23)c1 ; c1cc(-c2ccc3oc4ccccc4c3c2)cc(N(c2ccc(-c3cccc4sc5ccccc5c34)cc2)c2ccc3c(c2)sc2ccccc23)c1]

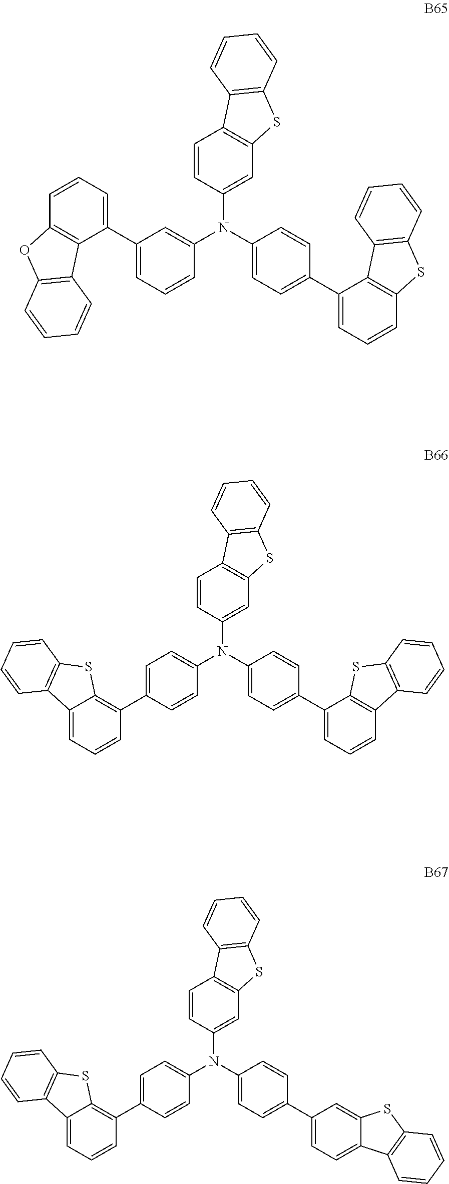 [SMILES: c1cc(-c2cccc3oc4ccccc4c23)cc(N(c2ccc(-c3cccc4sc5ccccc5c34)cc2)c2ccc3c(c2)sc2ccccc23)c1 ; c1ccc2c(c1)sc1cc(N(c3ccc(-c4cccc5c4sc4ccccc45)cc3)c3ccc(-c4cccc5c4sc4ccccc45)cc3)ccc12 ; c1ccc2c(c1)sc1cc(-c3ccc(N(c4ccc(-c5cccc6c5sc5ccccc56)cc4)c4ccc5c(c4)sc4ccccc45)cc3)ccc12]

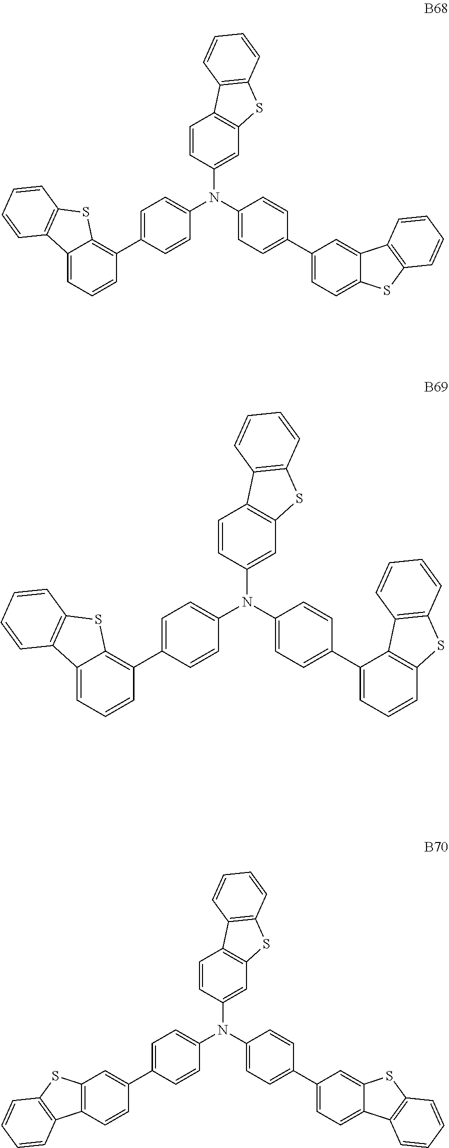 [SMILES: c1ccc2c(c1)sc1cc(N(c3ccc(-c4ccc5sc6ccccc6c5c4)cc3)c3ccc(-c4cccc5c4sc4ccccc45)cc3)ccc12 ; c1ccc2c(c1)sc1cc(N(c3ccc(-c4cccc5c4sc4ccccc45)cc3)c3ccc(-c4cccc5sc6ccccc6c45)cc3)ccc12 ; c1ccc2c(c1)sc1cc(-c3ccc(N(c4ccc(-c5ccc6c(c5)sc5ccccc56)cc4)c4ccc5c(c4)sc4ccccc45)cc3)ccc12]

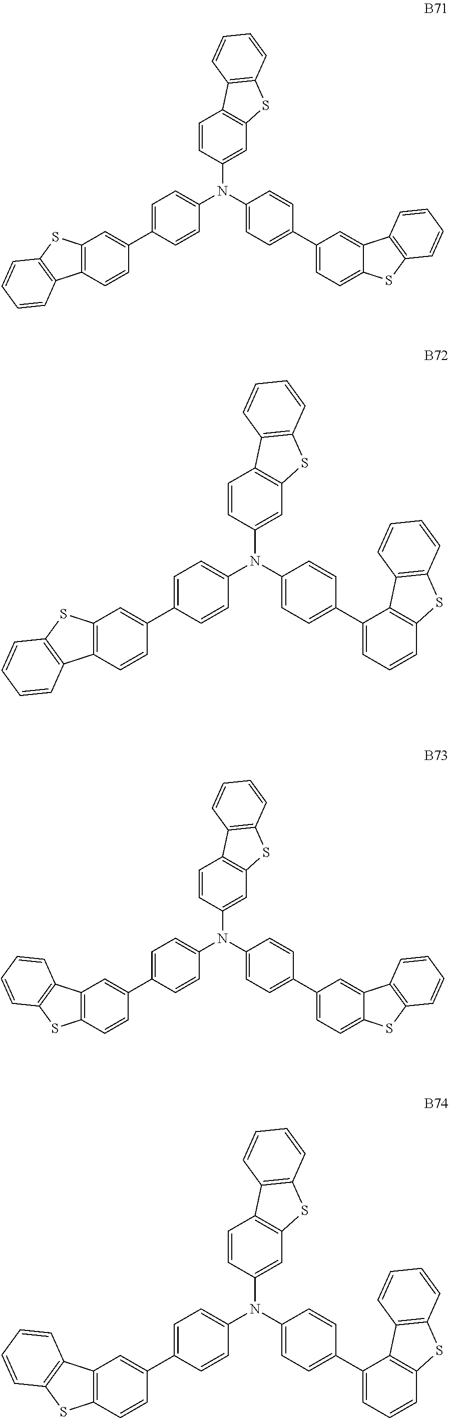 [SMILES: c1ccc2c(c1)sc1cc(-c3ccc(N(c4ccc(-c5ccc6sc7ccccc7c6c5)cc4)c4ccc5c(c4)sc4ccccc45)cc3)ccc12 ; c1ccc2c(c1)sc1cc(-c3ccc(N(c4ccc(-c5cccc6sc7ccccc7c56)cc4)c4ccc5c(c4)sc4ccccc45)cc3)ccc12 ; c1ccc2c(c1)sc1cc(N(c3ccc(-c4ccc5sc6ccccc6c5c4)cc3)c3ccc(-c4ccc5sc6ccccc6c5c4)cc3)ccc12 ; c1ccc2c(c1)sc1cc(N(c3ccc(-c4ccc5sc6ccccc6c5c4)cc3)c3ccc(-c4cccc5sc6ccccc6c45)cc3)ccc12]

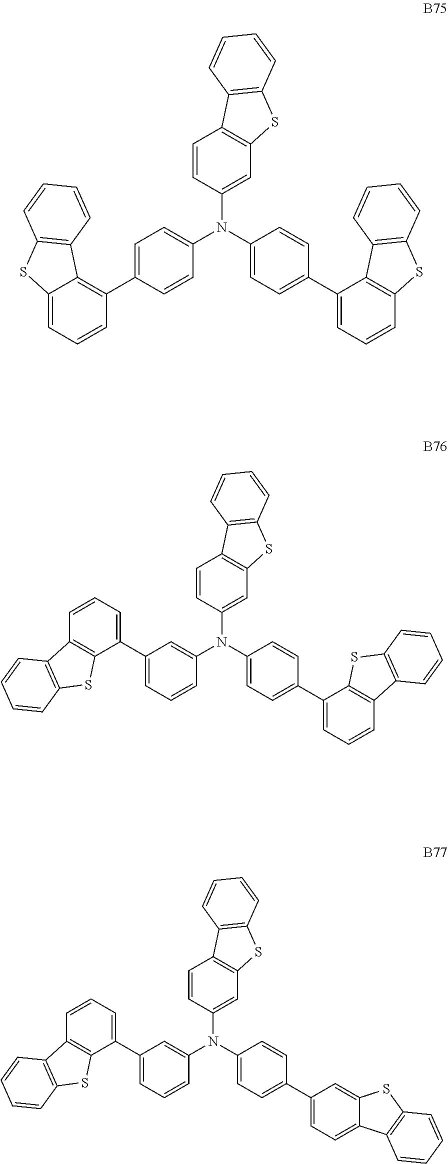 [SMILES: c1ccc2c(c1)sc1cc(N(c3ccc(-c4cccc5sc6ccccc6c45)cc3)c3ccc(-c4cccc5sc6ccccc6c45)cc3)ccc12 ; c1cc(-c2cccc3c2sc2ccccc23)cc(N(c2ccc(-c3cccc4c3sc3ccccc34)cc2)c2ccc3c(c2)sc2ccccc23)c1 ; c1cc(-c2cccc3c2sc2ccccc23)cc(N(c2ccc(-c3ccc4c(c3)sc3ccccc34)cc2)c2ccc3c(c2)sc2ccccc23)c1]

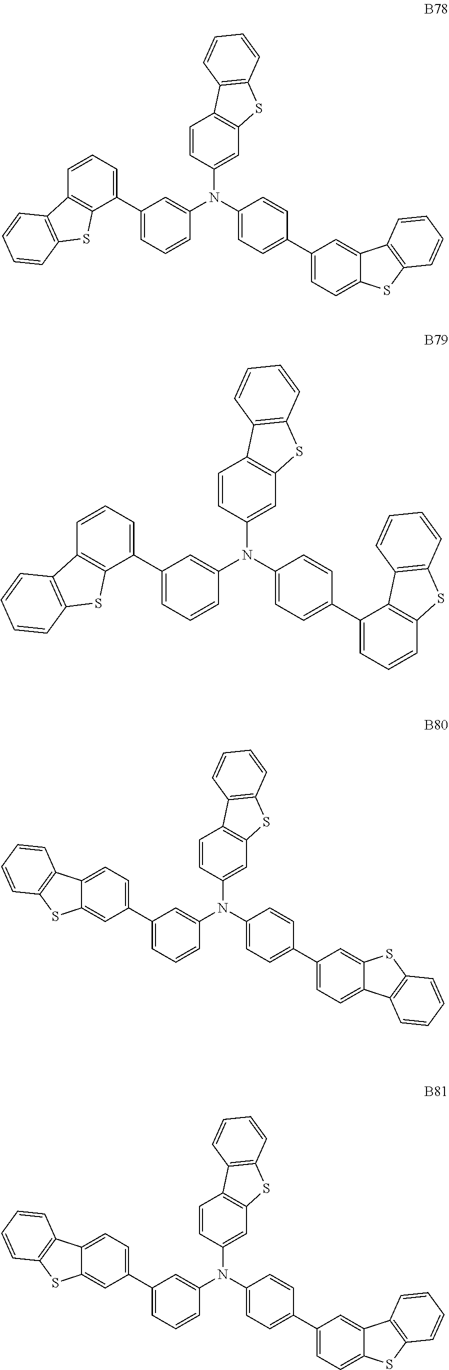 [SMILES: c1cc(-c2cccc3c2sc2ccccc23)cc(N(c2ccc(-c3ccc4sc5ccccc5c4c3)cc2)c2ccc3c(c2)sc2ccccc23)c1 ; c1cc(-c2cccc3c2sc2ccccc23)cc(N(c2ccc(-c3cccc4sc5ccccc5c34)cc2)c2ccc3c(c2)sc2ccccc23)c1 ; c1cc(-c2ccc3c(c2)sc2ccccc23)cc(N(c2ccc(-c3ccc4c(c3)sc3ccccc34)cc2)c2ccc3c(c2)sc2ccccc23)c1 ; c1cc(-c2ccc3c(c2)sc2ccccc23)cc(N(c2ccc(-c3ccc4sc5ccccc5c4c3)cc2)c2ccc3c(c2)sc2ccccc23)c1]

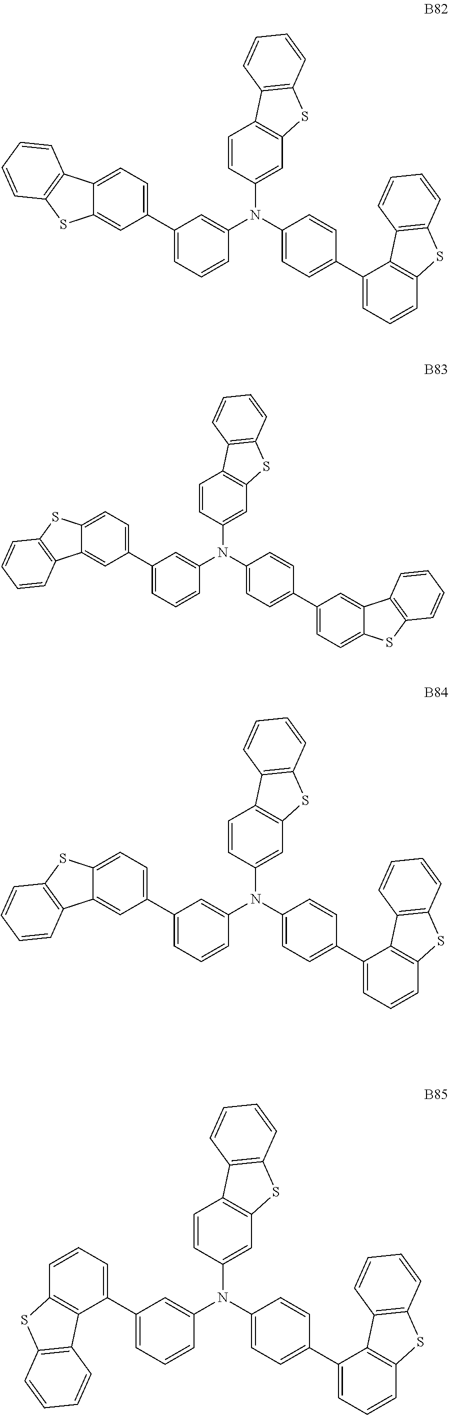 [SMILES: c1cc(-c2ccc3c(c2)sc2ccccc23)cc(N(c2ccc(-c3cccc4sc5ccccc5c34)cc2)c2ccc3c(c2)sc2ccccc23)c1 ; c1cc(-c2ccc3sc4ccccc4c3c2)cc(N(c2ccc(-c3ccc4sc5ccccc5c4c3)cc2)c2ccc3c(c2)sc2ccccc23)c1 ; c1cc(-c2ccc3sc4ccccc4c3c2)cc(N(c2ccc(-c3cccc4sc5ccccc5c34)cc2)c2ccc3c(c2)sc2ccccc23)c1 ; c1cc(-c2cccc3sc4ccccc4c23)cc(N(c2ccc(-c3cccc4sc5ccccc5c34)cc2)c2ccc3c(c2)sc2ccccc23)c1]

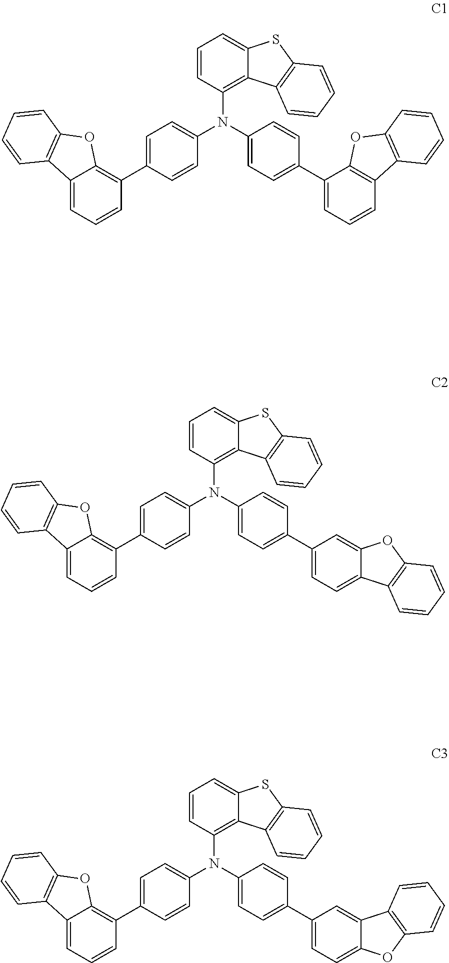 [SMILES: c1ccc2c(c1)oc1c(-c3ccc(N(c4ccc(-c5cccc6c5oc5ccccc56)cc4)c4cccc5sc6ccccc6c45)cc3)cccc12 ; c1ccc2c(c1)oc1cc(-c3ccc(N(c4ccc(-c5cccc6c5oc5ccccc56)cc4)c4cccc5sc6ccccc6c45)cc3)ccc12 ; c1ccc2c(c1)oc1ccc(-c3ccc(N(c4ccc(-c5cccc6c5oc5ccccc56)cc4)c4cccc5sc6ccccc6c45)cc3)cc12]

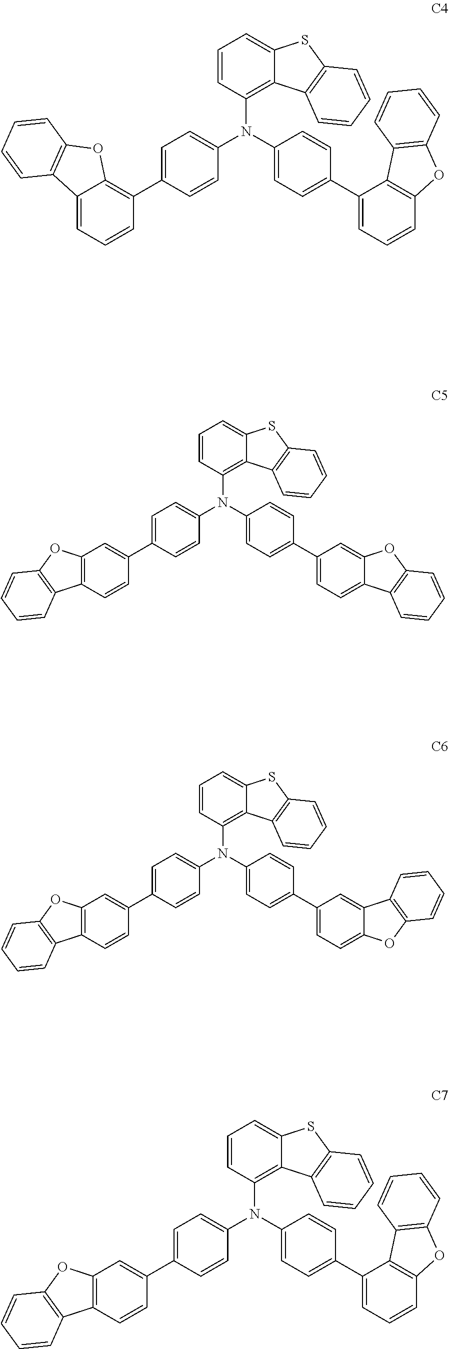 [SMILES: c1ccc2c(c1)oc1c(-c3ccc(N(c4ccc(-c5cccc6oc7ccccc7c56)cc4)c4cccc5sc6ccccc6c45)cc3)cccc12 ; c1ccc2c(c1)oc1cc(-c3ccc(N(c4ccc(-c5ccc6c(c5)oc5ccccc56)cc4)c4cccc5sc6ccccc6c45)cc3)ccc12 ; c1ccc2c(c1)oc1cc(-c3ccc(N(c4ccc(-c5ccc6oc7ccccc7c6c5)cc4)c4cccc5sc6ccccc6c45)cc3)ccc12 ; c1ccc2c(c1)oc1cc(-c3ccc(N(c4ccc(-c5cccc6oc7ccccc7c56)cc4)c4cccc5sc6ccccc6c45)cc3)ccc12]

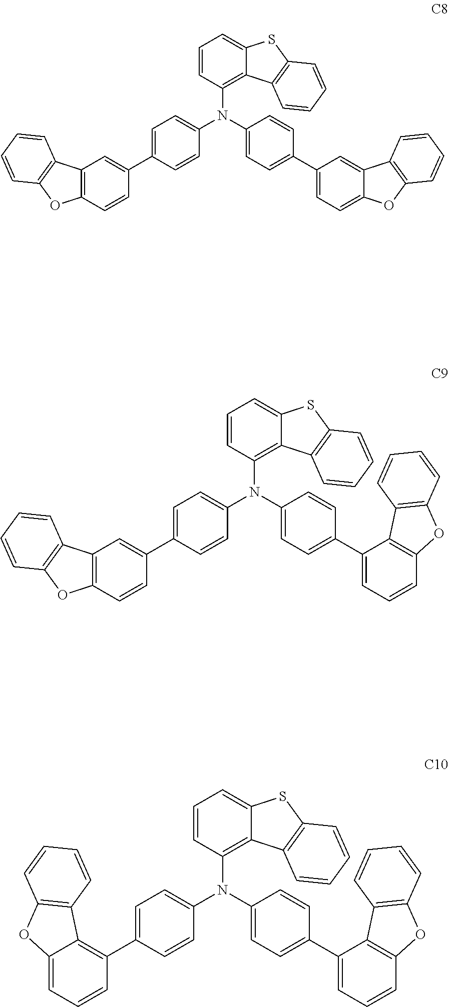 [SMILES: c1ccc2c(c1)oc1ccc(-c3ccc(N(c4ccc(-c5ccc6oc7ccccc7c6c5)cc4)c4cccc5sc6ccccc6c45)cc3)cc12 ; c1ccc2c(c1)oc1ccc(-c3ccc(N(c4ccc(-c5cccc6oc7ccccc7c56)cc4)c4cccc5sc6ccccc6c45)cc3)cc12 ; c1ccc2c(c1)oc1cccc(-c3ccc(N(c4ccc(-c5cccc6oc7ccccc7c56)cc4)c4cccc5sc6ccccc6c45)cc3)c12]

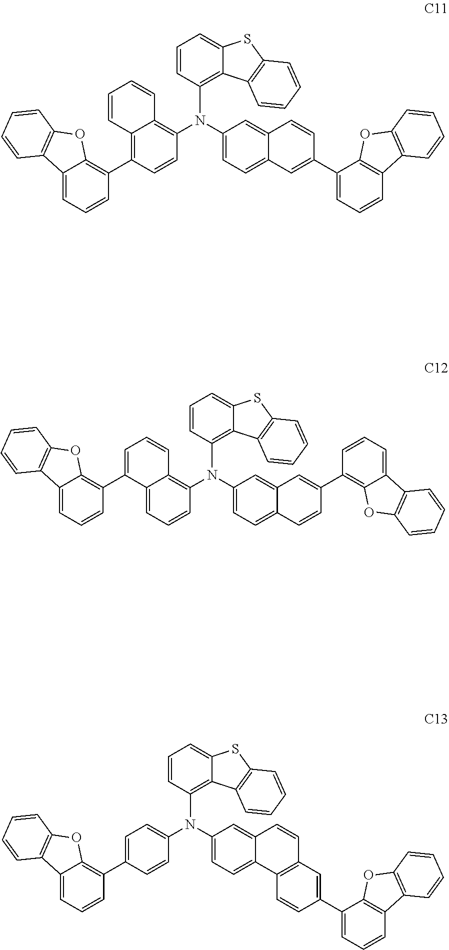 [SMILES: c1ccc2c(c1)oc1c(-c3ccc4cc(N(c5ccc(-c6cccc7c6oc6ccccc67)c6ccccc56)c5cccc6sc7ccccc7c56)ccc4c3)cccc12 ; c1ccc2c(c1)oc1c(-c3ccc4ccc(N(c5cccc6c(-c7cccc8c7oc7ccccc78)cccc56)c5cccc6sc7ccccc7c56)cc4c3)cccc12 ; c1ccc2c(c1)oc1c(-c3ccc(N(c4ccc5c(ccc6cc(-c7cccc8c7oc7ccccc78)ccc65)c4)c4cccc5sc6ccccc6c45)cc3)cccc12]

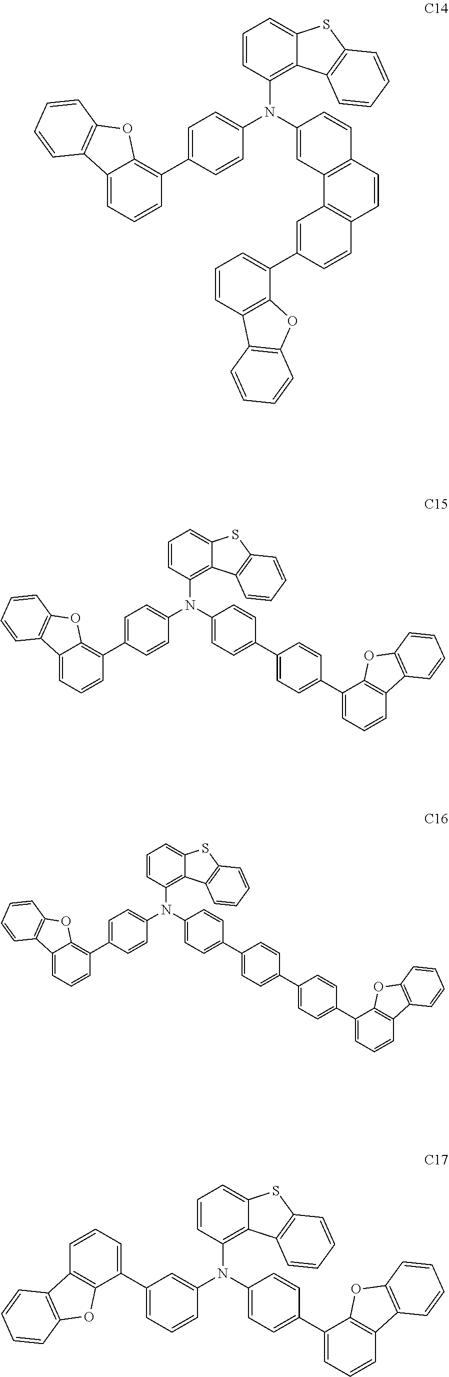 [SMILES: c1ccc2c(c1)oc1c(-c3ccc(N(c4ccc5ccc6ccc(-c7cccc8c7oc7ccccc78)cc6c5c4)c4cccc5sc6ccccc6c45)cc3)cccc12 ; c1ccc2c(c1)oc1c(-c3ccc(-c4ccc(N(c5ccc(-c6cccc7c6oc6ccccc67)cc5)c5cccc6sc7ccccc7c56)cc4)cc3)cccc12 ; c1ccc2c(c1)oc1c(-c3ccc(-c4ccc(-c5ccc(N(c6ccc(-c7cccc8c7oc7ccccc78)cc6)c6cccc7sc8ccccc8c67)cc5)cc4)cc3)cccc12 ; c1cc(-c2cccc3c2oc2ccccc23)cc(N(c2ccc(-c3cccc4c3oc3ccccc34)cc2)c2cccc3sc4ccccc4c23)c1]

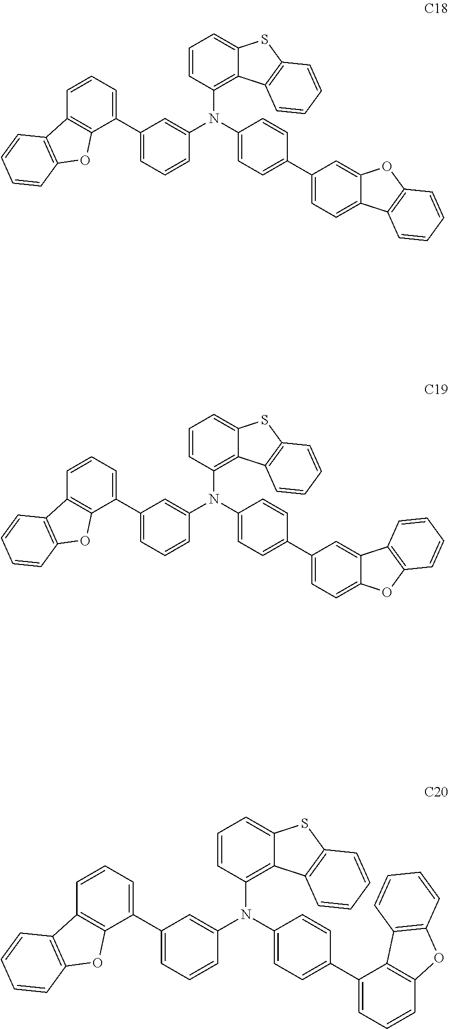 [SMILES: c1cc(-c2cccc3c2oc2ccccc23)cc(N(c2ccc(-c3ccc4c(c3)oc3ccccc34)cc2)c2cccc3sc4ccccc4c23)c1 ; c1cc(-c2cccc3c2oc2ccccc23)cc(N(c2ccc(-c3ccc4oc5ccccc5c4c3)cc2)c2cccc3sc4ccccc4c23)c1 ; c1cc(-c2cccc3c2oc2ccccc23)cc(N(c2ccc(-c3cccc4oc5ccccc5c34)cc2)c2cccc3sc4ccccc4c23)c1]

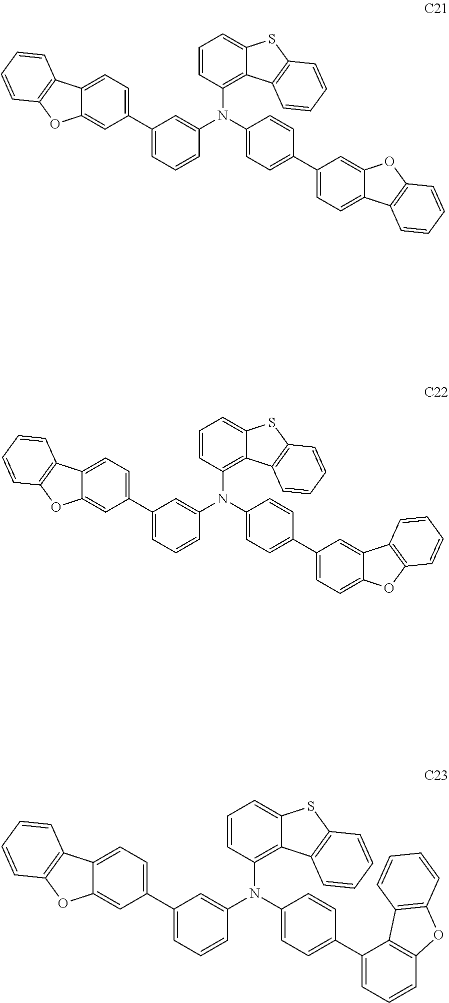 [SMILES: c1cc(-c2ccc3c(c2)oc2ccccc23)cc(N(c2ccc(-c3ccc4c(c3)oc3ccccc34)cc2)c2cccc3sc4ccccc4c23)c1 ; c1cc(-c2ccc3c(c2)oc2ccccc23)cc(N(c2ccc(-c3ccc4oc5ccccc5c4c3)cc2)c2cccc3sc4ccccc4c23)c1 ; c1cc(-c2ccc3c(c2)oc2ccccc23)cc(N(c2ccc(-c3cccc4oc5ccccc5c34)cc2)c2cccc3sc4ccccc4c23)c1]

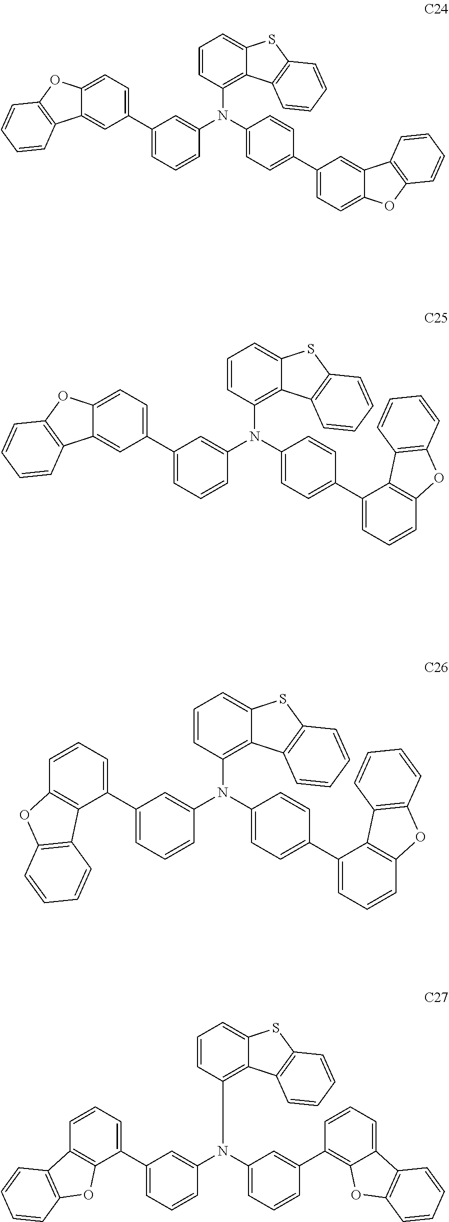 [SMILES: c1cc(-c2ccc3oc4ccccc4c3c2)cc(N(c2ccc(-c3ccc4oc5ccccc5c4c3)cc2)c2cccc3sc4ccccc4c23)c1 ; c1cc(-c2ccc3oc4ccccc4c3c2)cc(N(c2ccc(-c3cccc4oc5ccccc5c34)cc2)c2cccc3sc4ccccc4c23)c1 ; c1cc(-c2cccc3oc4ccccc4c23)cc(N(c2ccc(-c3cccc4oc5ccccc5c34)cc2)c2cccc3sc4ccccc4c23)c1 ; c1cc(-c2cccc3c2oc2ccccc23)cc(N(c2cccc(-c3cccc4c3oc3ccccc34)c2)c2cccc3sc4ccccc4c23)c1]

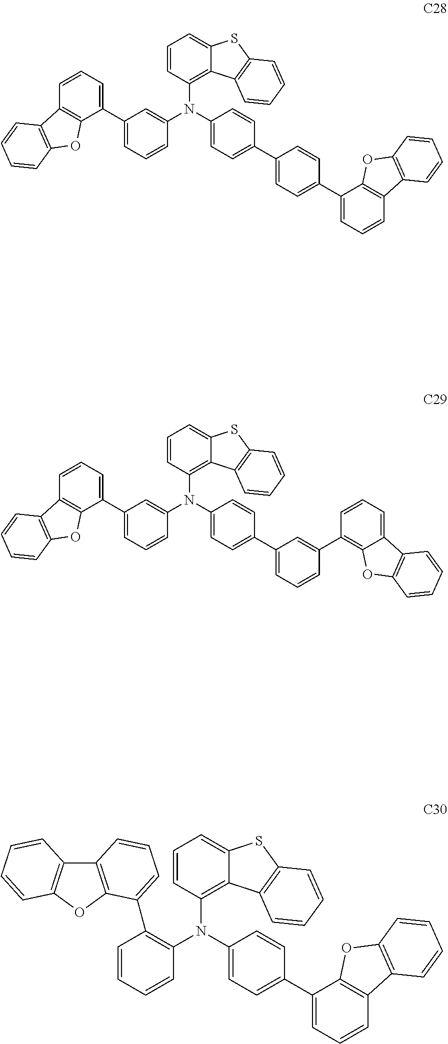 [SMILES: c1cc(-c2cccc3c2oc2ccccc23)cc(N(c2ccc(-c3ccc(-c4cccc5c4oc4ccccc45)cc3)cc2)c2cccc3sc4ccccc4c23)c1 ; c1cc(-c2ccc(N(c3cccc(-c4cccc5c4oc4ccccc45)c3)c3cccc4sc5ccccc5c34)cc2)cc(-c2cccc3c2oc2ccccc23)c1 ; c1ccc(N(c2ccc(-c3cccc4c3oc3ccccc34)cc2)c2cccc3sc4ccccc4c23)c(-c2cccc3c2oc2ccccc23)c1]

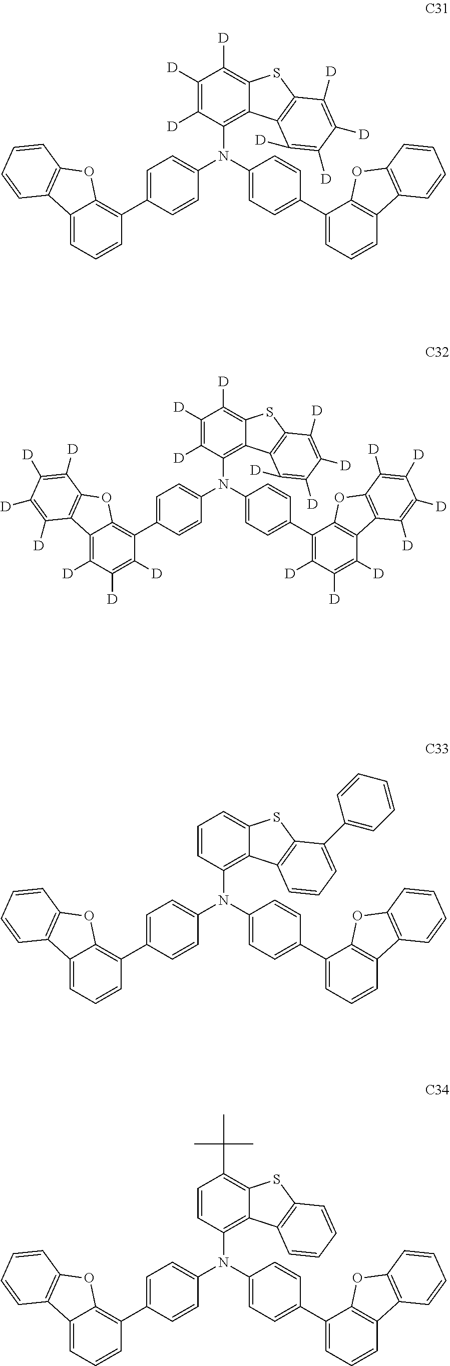

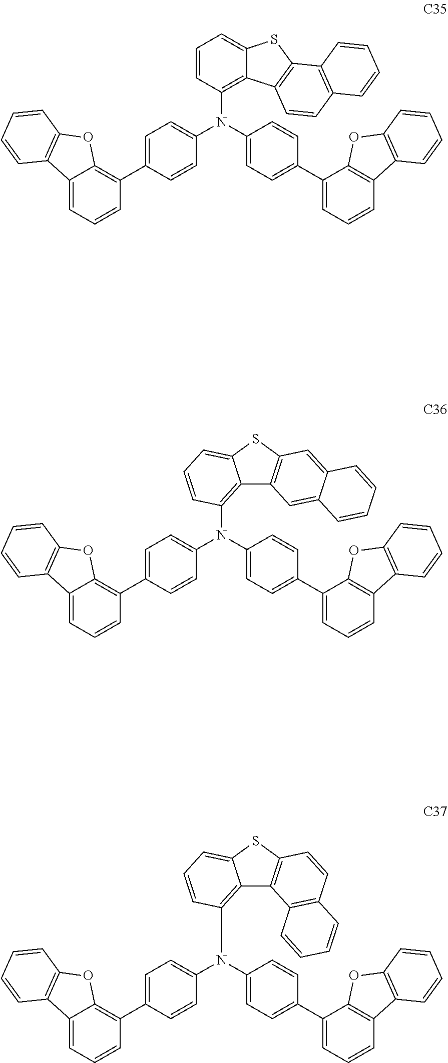 [SMILES: c1ccc2c(c1)ccc1c2sc2cccc(N(c3ccc(-c4cccc5c4oc4ccccc45)cc3)c3ccc(-c4cccc5c4oc4ccccc45)cc3)c21 ; c1ccc2cc3c(cc2c1)sc1cccc(N(c2ccc(-c4cccc5c4oc4ccccc45)cc2)c2ccc(-c4cccc5c4oc4ccccc45)cc2)c13 ; c1ccc2c(c1)ccc1sc3cccc(N(c4ccc(-c5cccc6c5oc5ccccc56)cc4)c4ccc(-c5cccc6c5oc5ccccc56)cc4)c3c12]

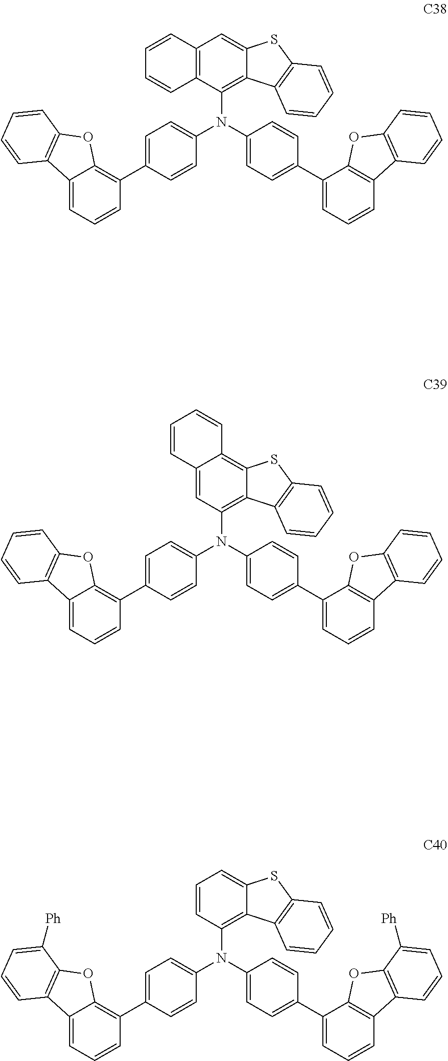 [SMILES: c1ccc2c(N(c3ccc(-c4cccc5c4oc4ccccc45)cc3)c3ccc(-c4cccc5c4oc4ccccc45)cc3)c3c(cc2c1)sc1ccccc13 ; c1ccc2c(c1)cc(N(c1ccc(-c3cccc4c3oc3ccccc34)cc1)c1ccc(-c3cccc4c3oc3ccccc34)cc1)c1c3ccccc3sc21 ; c1ccc(-c2cccc3c2oc2c(-c4ccc(N(c5ccc(-c6cccc7c6oc6c(-c8ccccc8)cccc67)cc5)c5cccc6sc7ccccc7c56)cc4)cccc23)cc1]

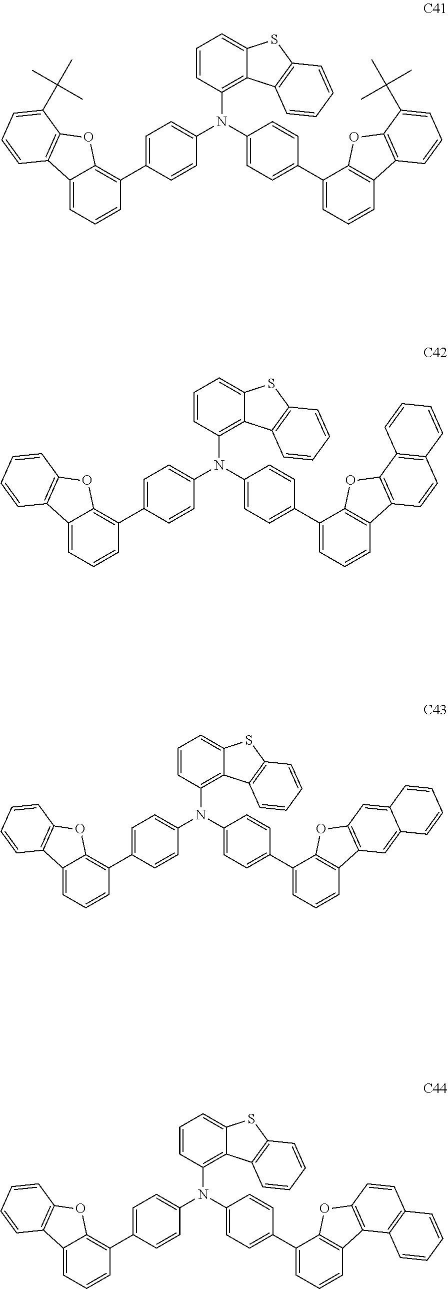 [SMILES: CC(C)(C)c1cccc2c1oc1c(-c3ccc(N(c4ccc(-c5cccc6c5oc5c(C(C)(C)C)cccc56)cc4)c4cccc5sc6ccccc6c45)cc3)cccc12 ; c1ccc2c(c1)ccc1c3cccc(-c4ccc(N(c5ccc(-c6cccc7c6oc6ccccc67)cc5)c5cccc6sc7ccccc7c56)cc4)c3oc21 ; c1ccc2cc3c(cc2c1)oc1c(-c2ccc(N(c4ccc(-c5cccc6c5oc5ccccc56)cc4)c4cccc5sc6ccccc6c45)cc2)cccc13 ; c1ccc2c(c1)ccc1oc3c(-c4ccc(N(c5ccc(-c6cccc7c6oc6ccccc67)cc5)c5cccc6sc7ccccc7c56)cc4)cccc3c12]

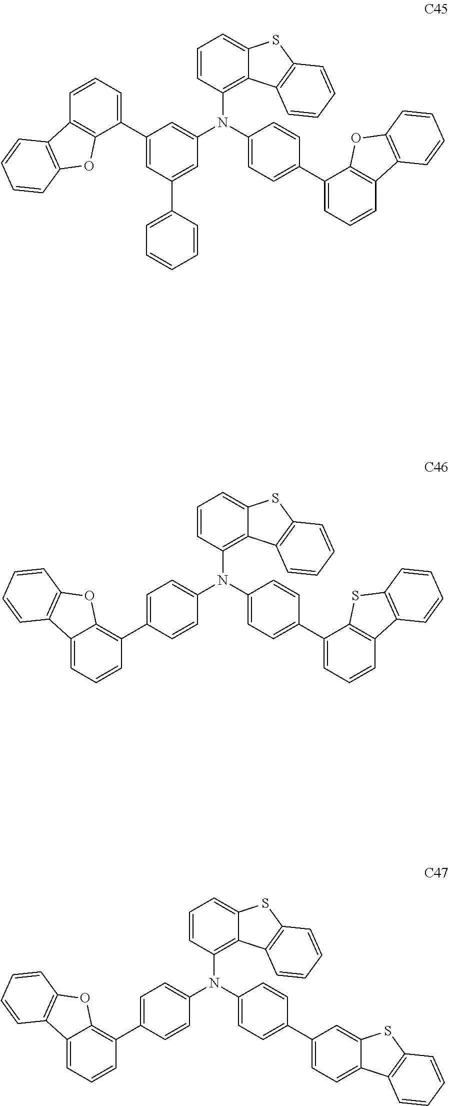 [SMILES: c1ccc(-c2cc(-c3cccc4c3oc3ccccc34)cc(N(c3ccc(-c4cccc5c4oc4ccccc45)cc3)c3cccc4sc5ccccc5c34)c2)cc1 ; c1ccc2c(c1)oc1c(-c3ccc(N(c4ccc(-c5cccc6c5sc5ccccc56)cc4)c4cccc5sc6ccccc6c45)cc3)cccc12 ; c1ccc2c(c1)oc1c(-c3ccc(N(c4ccc(-c5ccc6c(c5)sc5ccccc56)cc4)c4cccc5sc6ccccc6c45)cc3)cccc12]

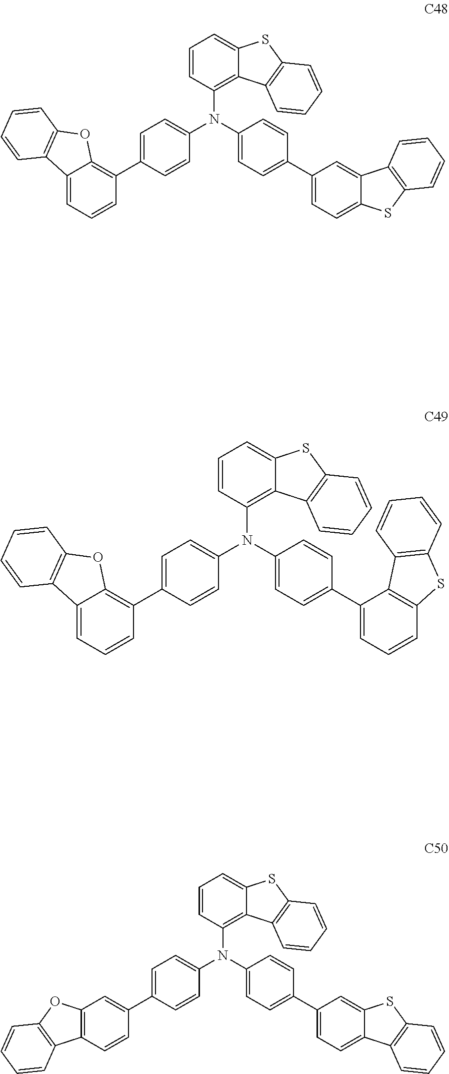 [SMILES: c1ccc2c(c1)oc1c(-c3ccc(N(c4ccc(-c5ccc6sc7ccccc7c6c5)cc4)c4cccc5sc6ccccc6c45)cc3)cccc12 ; c1ccc2c(c1)oc1c(-c3ccc(N(c4ccc(-c5cccc6sc7ccccc7c56)cc4)c4cccc5sc6ccccc6c45)cc3)cccc12 ; c1ccc2c(c1)oc1cc(-c3ccc(N(c4ccc(-c5ccc6c(c5)sc5ccccc56)cc4)c4cccc5sc6ccccc6c45)cc3)ccc12]

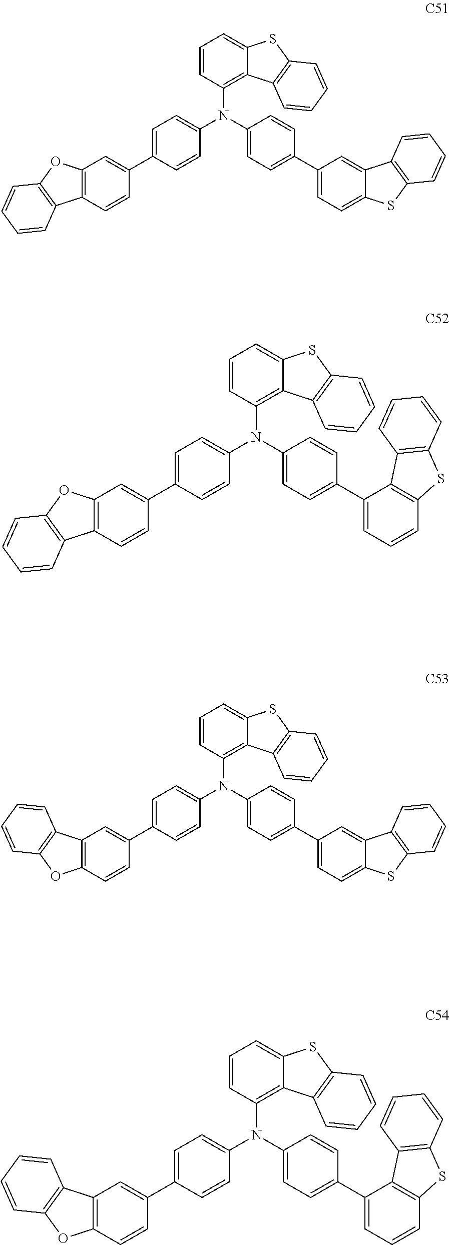 [SMILES: c1ccc2c(c1)oc1cc(-c3ccc(N(c4ccc(-c5ccc6sc7ccccc7c6c5)cc4)c4cccc5sc6ccccc6c45)cc3)ccc12 ; c1ccc2c(c1)oc1cc(-c3ccc(N(c4ccc(-c5cccc6sc7ccccc7c56)cc4)c4cccc5sc6ccccc6c45)cc3)ccc12 ; c1ccc2c(c1)oc1ccc(-c3ccc(N(c4ccc(-c5ccc6sc7ccccc7c6c5)cc4)c4cccc5sc6ccccc6c45)cc3)cc12 ; c1ccc2c(c1)oc1ccc(-c3ccc(N(c4ccc(-c5cccc6sc7ccccc7c56)cc4)c4cccc5sc6ccccc6c45)cc3)cc12]

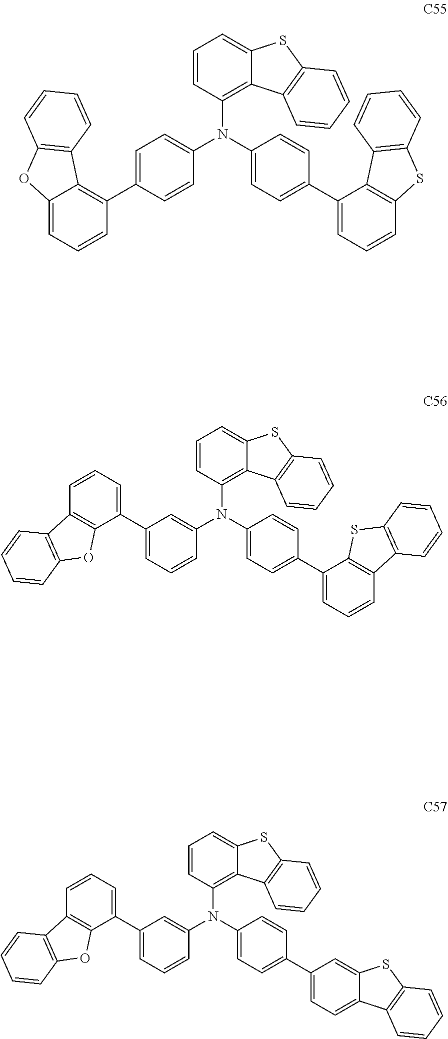 [SMILES: c1ccc2c(c1)oc1cccc(-c3ccc(N(c4ccc(-c5cccc6sc7ccccc7c56)cc4)c4cccc5sc6ccccc6c45)cc3)c12 ; c1cc(-c2cccc3c2oc2ccccc23)cc(N(c2ccc(-c3cccc4c3sc3ccccc34)cc2)c2cccc3sc4ccccc4c23)c1 ; c1cc(-c2cccc3c2oc2ccccc23)cc(N(c2ccc(-c3ccc4c(c3)sc3ccccc34)cc2)c2cccc3sc4ccccc4c23)c1]

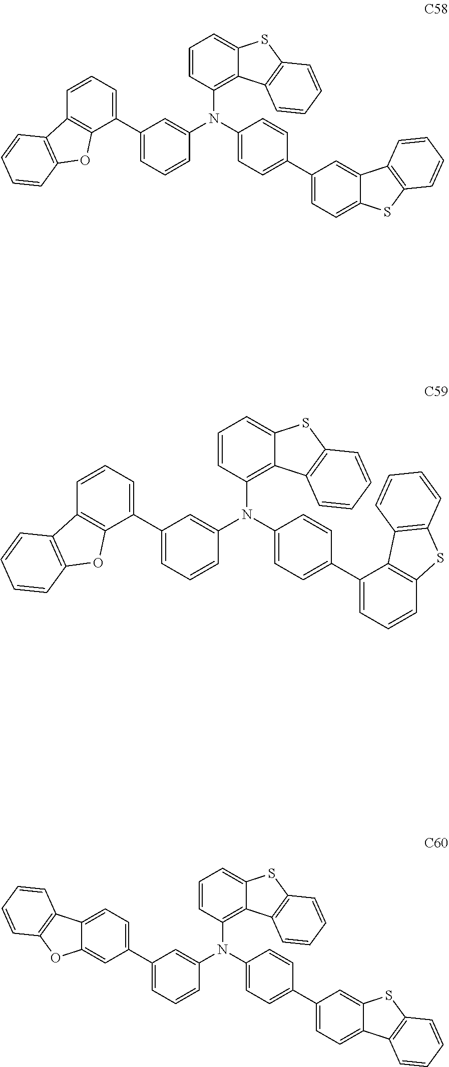 [SMILES: c1cc(-c2cccc3c2oc2ccccc23)cc(N(c2ccc(-c3ccc4sc5ccccc5c4c3)cc2)c2cccc3sc4ccccc4c23)c1 ; c1cc(-c2cccc3c2oc2ccccc23)cc(N(c2ccc(-c3cccc4sc5ccccc5c34)cc2)c2cccc3sc4ccccc4c23)c1 ; c1cc(-c2ccc3c(c2)oc2ccccc23)cc(N(c2ccc(-c3ccc4c(c3)sc3ccccc34)cc2)c2cccc3sc4ccccc4c23)c1]

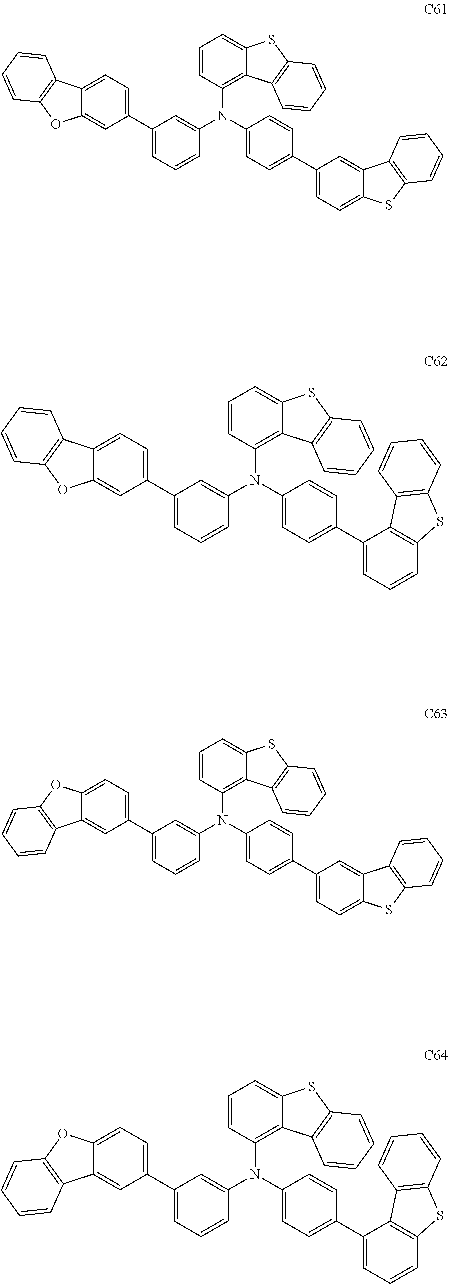 [SMILES: c1cc(-c2ccc3c(c2)oc2ccccc23)cc(N(c2ccc(-c3ccc4sc5ccccc5c4c3)cc2)c2cccc3sc4ccccc4c23)c1 ; c1cc(-c2ccc3c(c2)oc2ccccc23)cc(N(c2ccc(-c3cccc4sc5ccccc5c34)cc2)c2cccc3sc4ccccc4c23)c1 ; c1cc(-c2ccc3oc4ccccc4c3c2)cc(N(c2ccc(-c3ccc4sc5ccccc5c4c3)cc2)c2cccc3sc4ccccc4c23)c1 ; c1cc(-c2ccc3oc4ccccc4c3c2)cc(N(c2ccc(-c3cccc4sc5ccccc5c34)cc2)c2cccc3sc4ccccc4c23)c1]

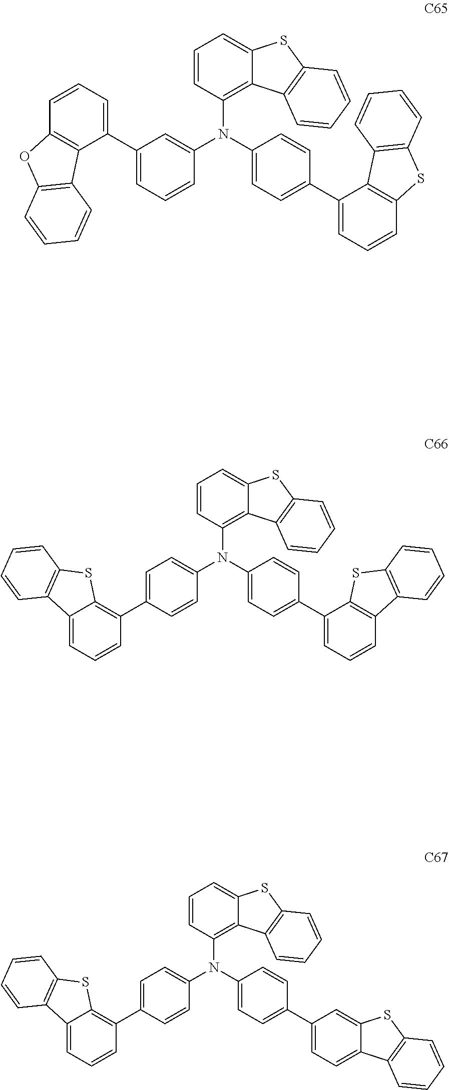 [SMILES: c1cc(-c2cccc3oc4ccccc4c23)cc(N(c2ccc(-c3cccc4sc5ccccc5c34)cc2)c2cccc3sc4ccccc4c23)c1 ; c1ccc2c(c1)sc1c(-c3ccc(N(c4ccc(-c5cccc6c5sc5ccccc56)cc4)c4cccc5sc6ccccc6c45)cc3)cccc12 ; c1ccc2c(c1)sc1cc(-c3ccc(N(c4ccc(-c5cccc6c5sc5ccccc56)cc4)c4cccc5sc6ccccc6c45)cc3)ccc12]

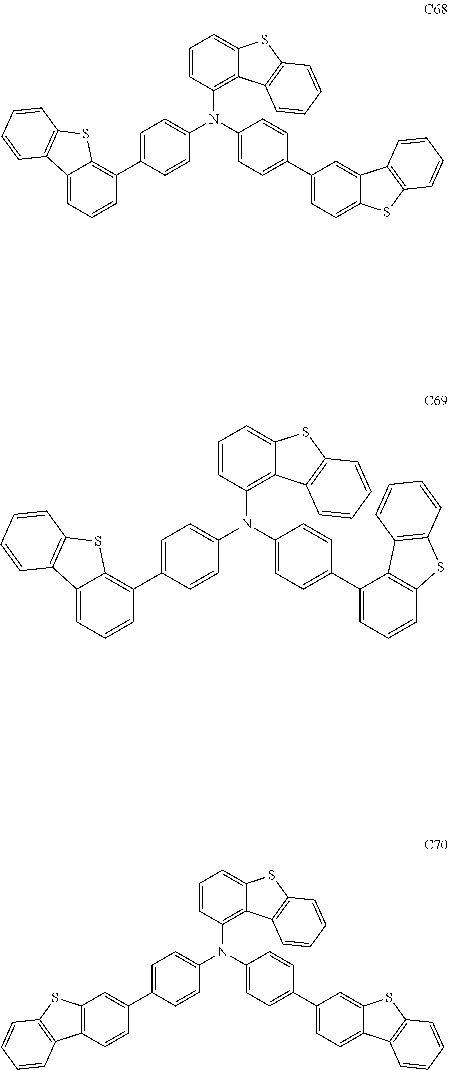 [SMILES: c1ccc2c(c1)sc1ccc(-c3ccc(N(c4ccc(-c5cccc6c5sc5ccccc56)cc4)c4cccc5sc6ccccc6c45)cc3)cc12 ; c1ccc2c(c1)sc1c(-c3ccc(N(c4ccc(-c5cccc6sc7ccccc7c56)cc4)c4cccc5sc6ccccc6c45)cc3)cccc12 ; c1ccc2c(c1)sc1cc(-c3ccc(N(c4ccc(-c5ccc6c(c5)sc5ccccc56)cc4)c4cccc5sc6ccccc6c45)cc3)ccc12]

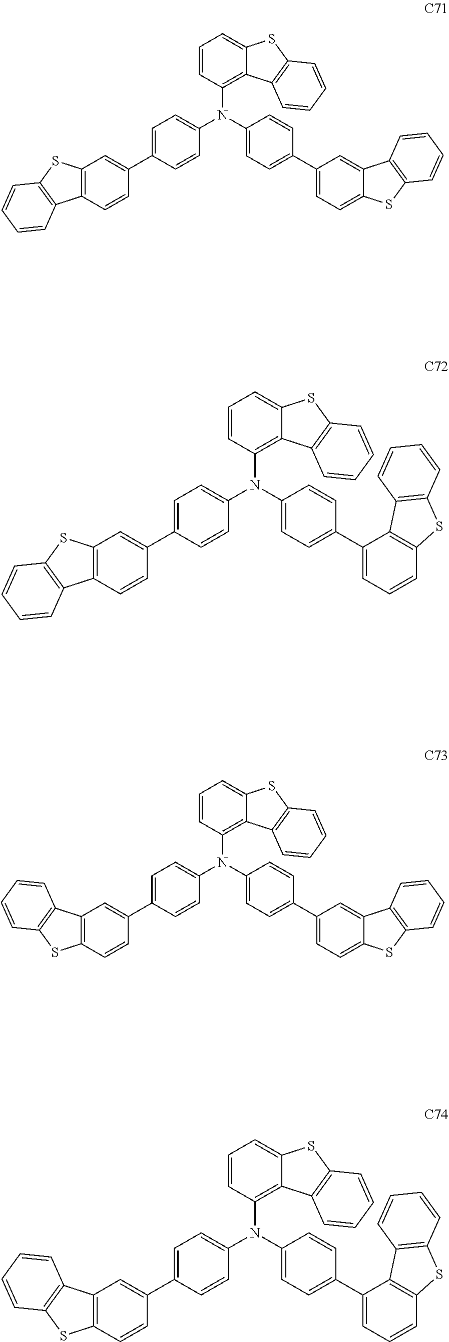 [SMILES: c1ccc2c(c1)sc1cc(-c3ccc(N(c4ccc(-c5ccc6sc7ccccc7c6c5)cc4)c4cccc5sc6ccccc6c45)cc3)ccc12 ; c1ccc2c(c1)sc1cc(-c3ccc(N(c4ccc(-c5cccc6sc7ccccc7c56)cc4)c4cccc5sc6ccccc6c45)cc3)ccc12 ; c1ccc2c(c1)sc1ccc(-c3ccc(N(c4ccc(-c5ccc6sc7ccccc7c6c5)cc4)c4cccc5sc6ccccc6c45)cc3)cc12 ; c1ccc2c(c1)sc1ccc(-c3ccc(N(c4ccc(-c5cccc6sc7ccccc7c56)cc4)c4cccc5sc6ccccc6c45)cc3)cc12]

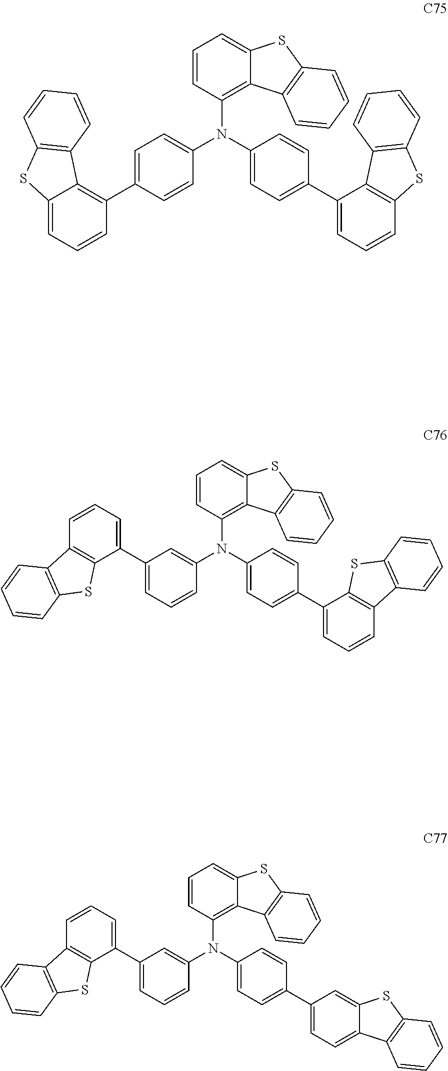 [SMILES: c1ccc2c(c1)sc1cccc(-c3ccc(N(c4ccc(-c5cccc6sc7ccccc7c56)cc4)c4cccc5sc6ccccc6c45)cc3)c12 ; c1cc(-c2cccc3c2sc2ccccc23)cc(N(c2ccc(-c3cccc4c3sc3ccccc34)cc2)c2cccc3sc4ccccc4c23)c1 ; c1cc(-c2cccc3c2sc2ccccc23)cc(N(c2ccc(-c3ccc4c(c3)sc3ccccc34)cc2)c2cccc3sc4ccccc4c23)c1]

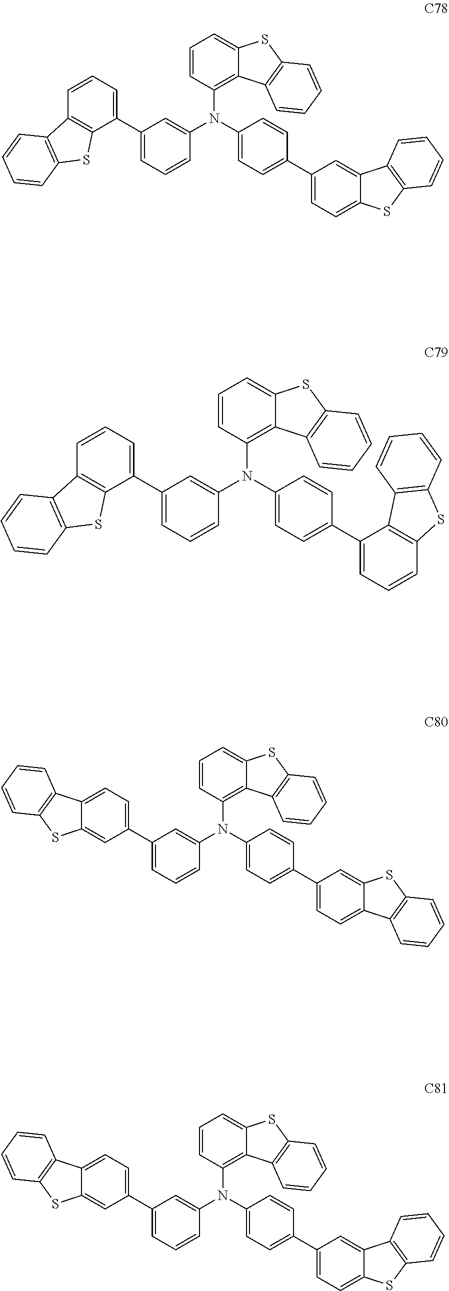 [SMILES: c1cc(-c2cccc3c2sc2ccccc23)cc(N(c2ccc(-c3ccc4sc5ccccc5c4c3)cc2)c2cccc3sc4ccccc4c23)c1 ; c1cc(-c2cccc3c2sc2ccccc23)cc(N(c2ccc(-c3cccc4sc5ccccc5c34)cc2)c2cccc3sc4ccccc4c23)c1 ; c1cc(-c2ccc3c(c2)sc2ccccc23)cc(N(c2ccc(-c3ccc4c(c3)sc3ccccc34)cc2)c2cccc3sc4ccccc4c23)c1 ; c1cc(-c2ccc3c(c2)sc2ccccc23)cc(N(c2ccc(-c3ccc4sc5ccccc5c4c3)cc2)c2cccc3sc4ccccc4c23)c1]

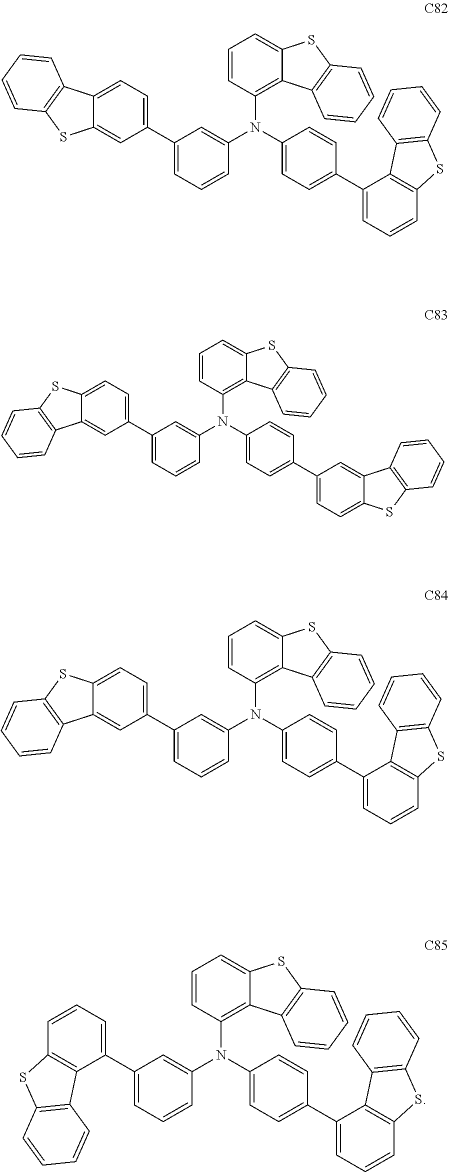 [SMILES: c1cc(-c2ccc3c(c2)sc2ccccc23)cc(N(c2ccc(-c3cccc4sc5ccccc5c34)cc2)c2cccc3sc4ccccc4c23)c1 ; c1cc(-c2ccc3sc4ccccc4c3c2)cc(N(c2ccc(-c3ccc4sc5ccccc5c4c3)cc2)c2cccc3sc4ccccc4c23)c1 ; c1cc(-c2ccc3sc4ccccc4c3c2)cc(N(c2ccc(-c3cccc4sc5ccccc5c34)cc2)c2cccc3sc4ccccc4c23)c1 ; c1cc(-c2cccc3sc4ccccc4c23)cc(N(c2ccc(-c3cccc4sc5ccccc5c34)cc2)c2cccc3sc4ccccc4c23)c1]

20. The amine compound of claim 12, wherein the amine compound represented by Formula 1 is selected from Compound Group 2: 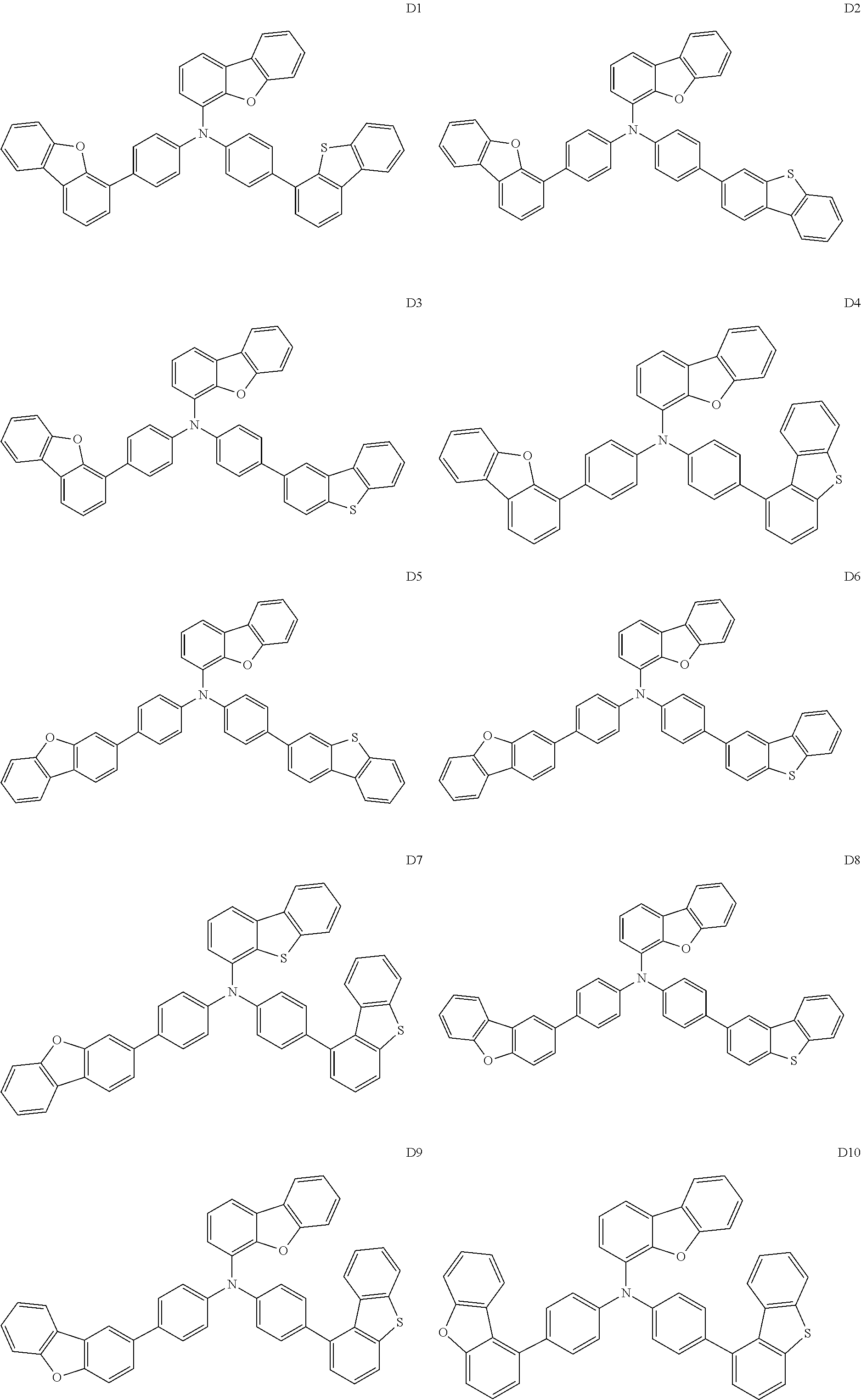 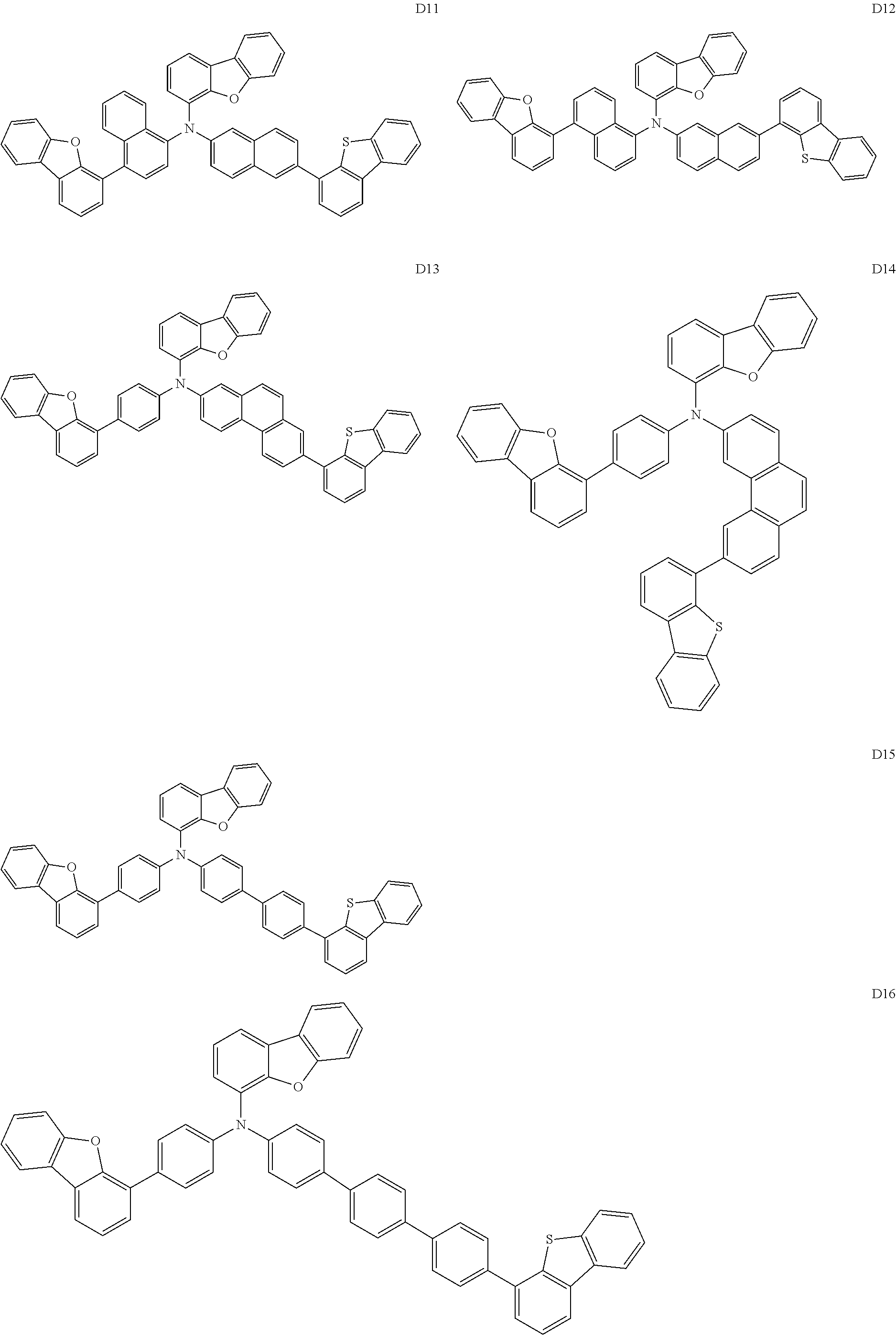 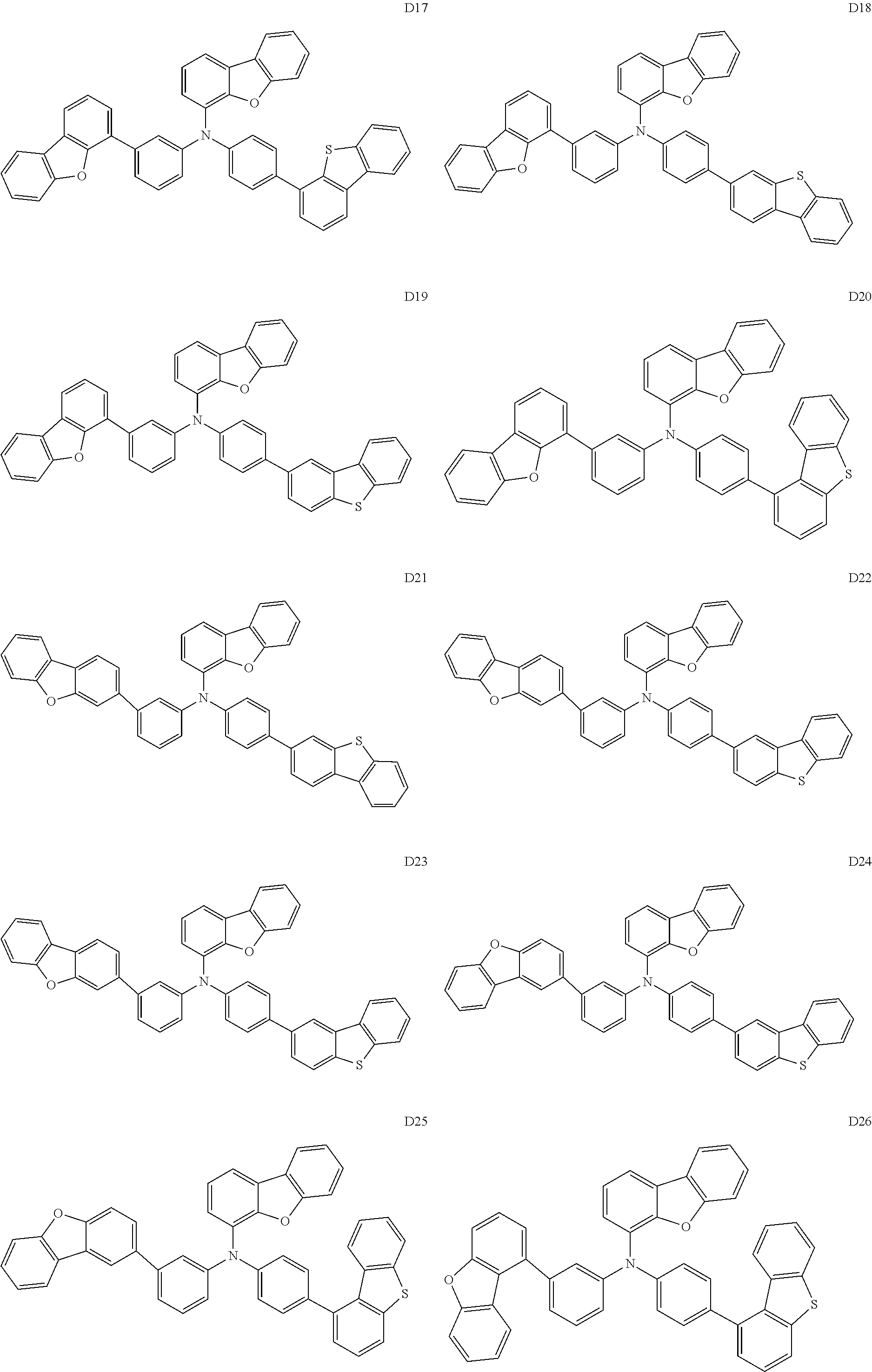 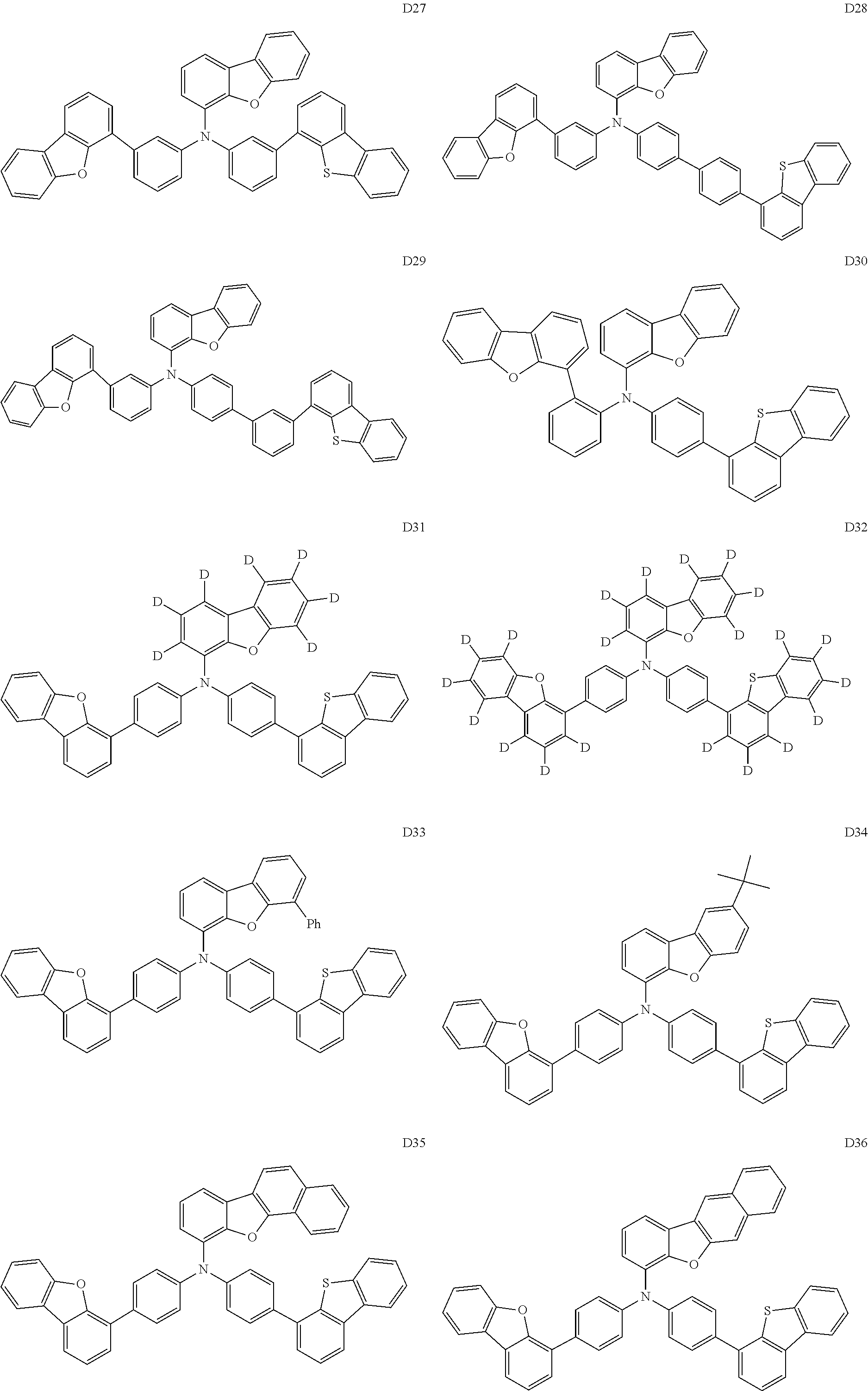 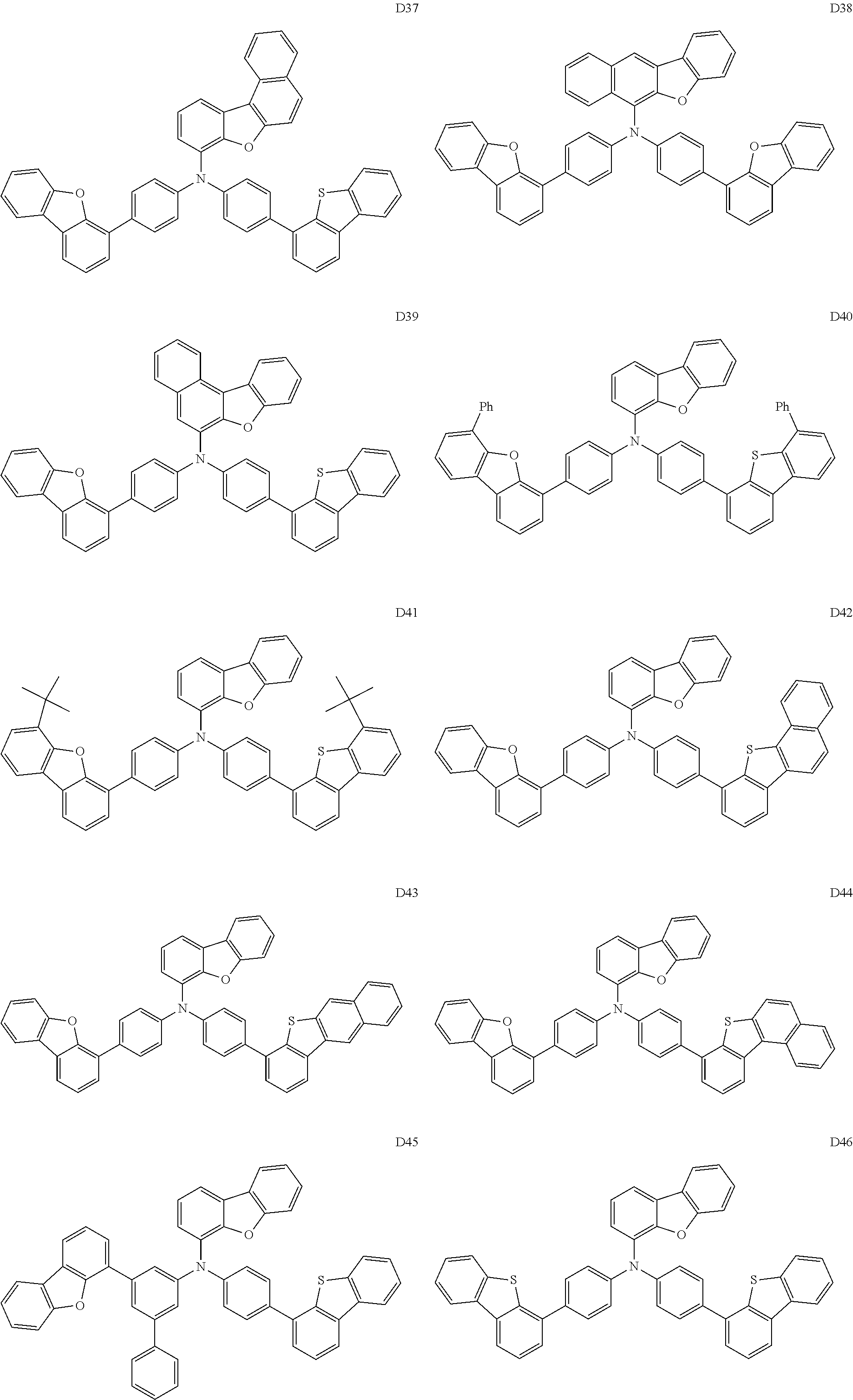 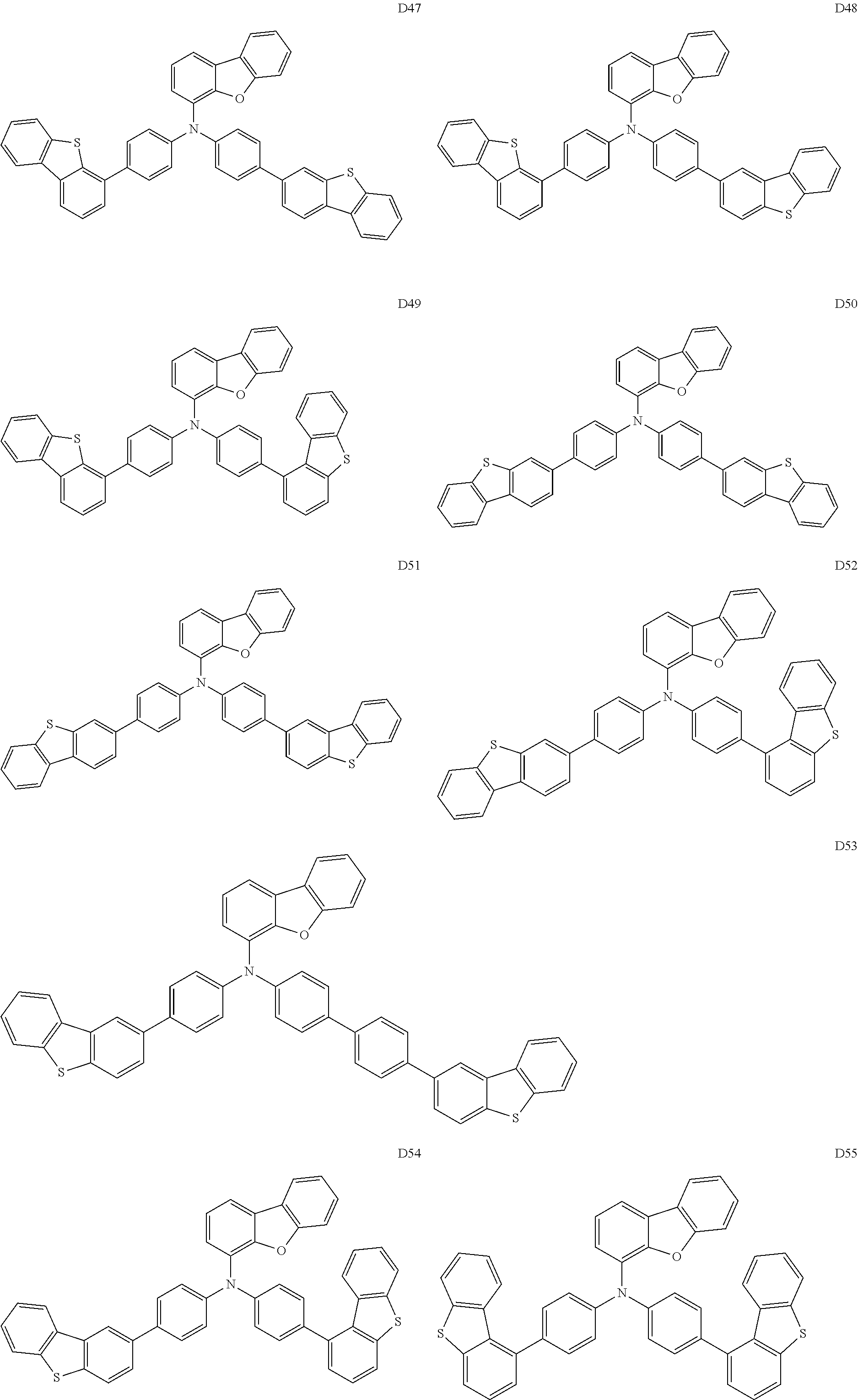 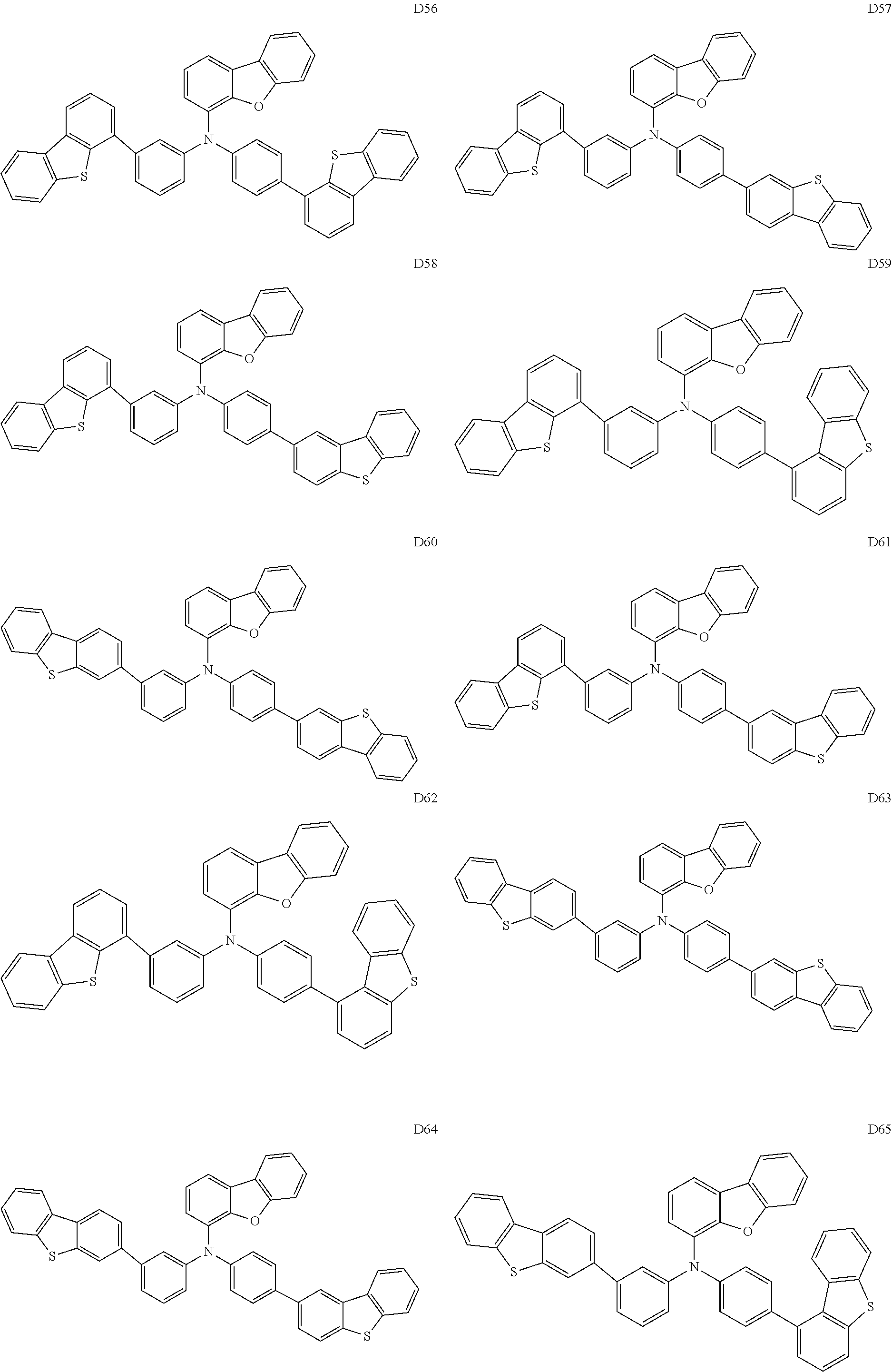 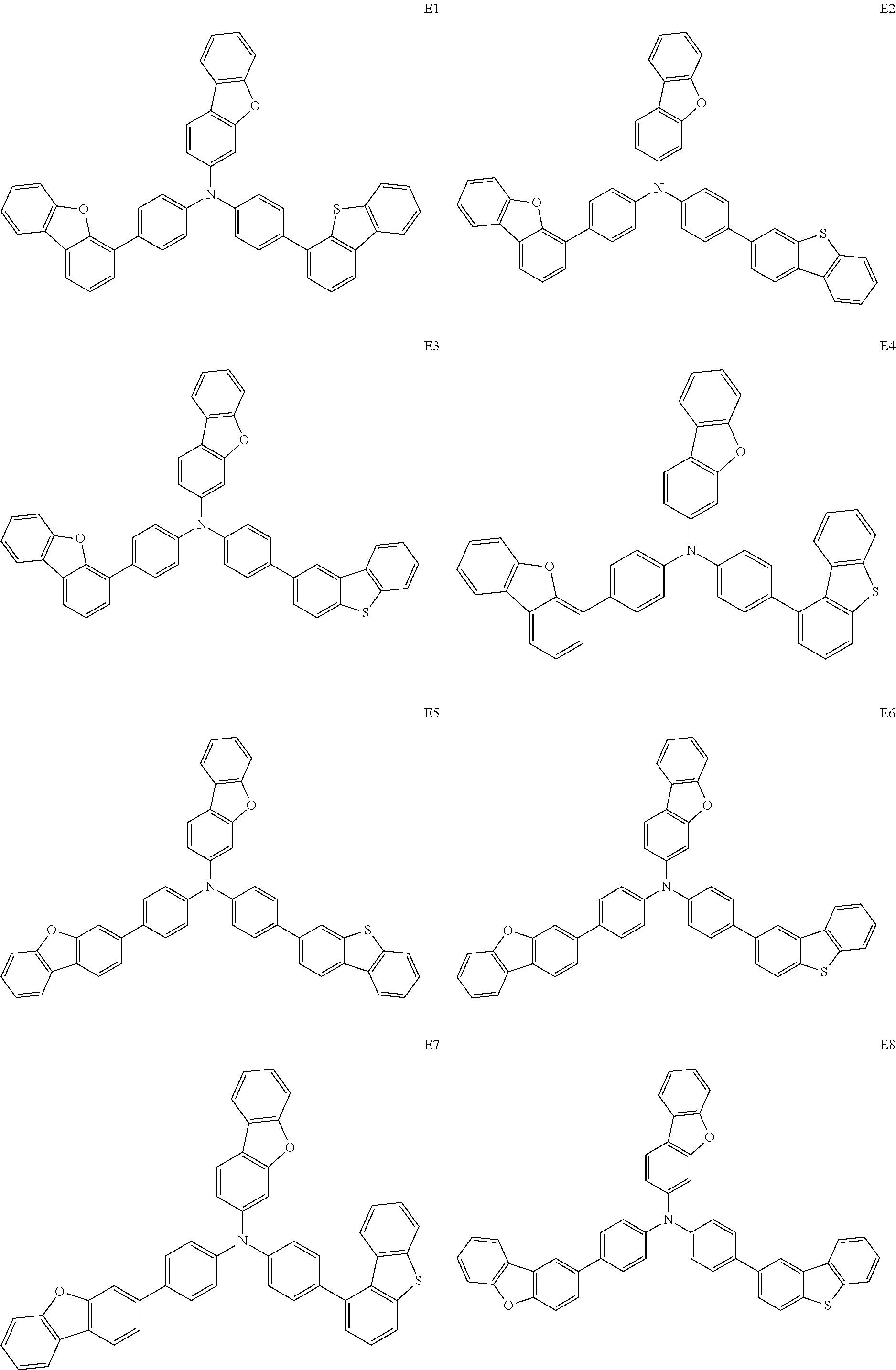 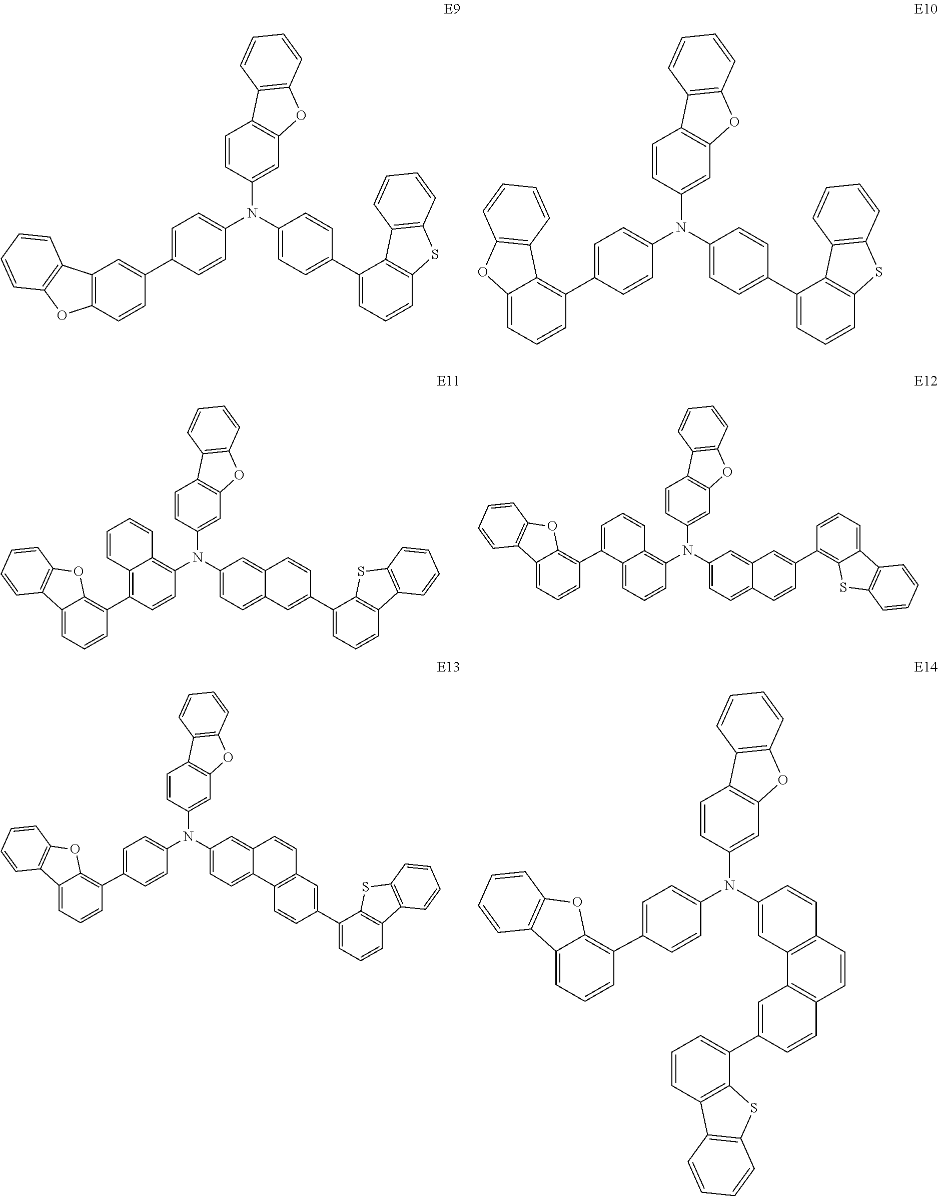 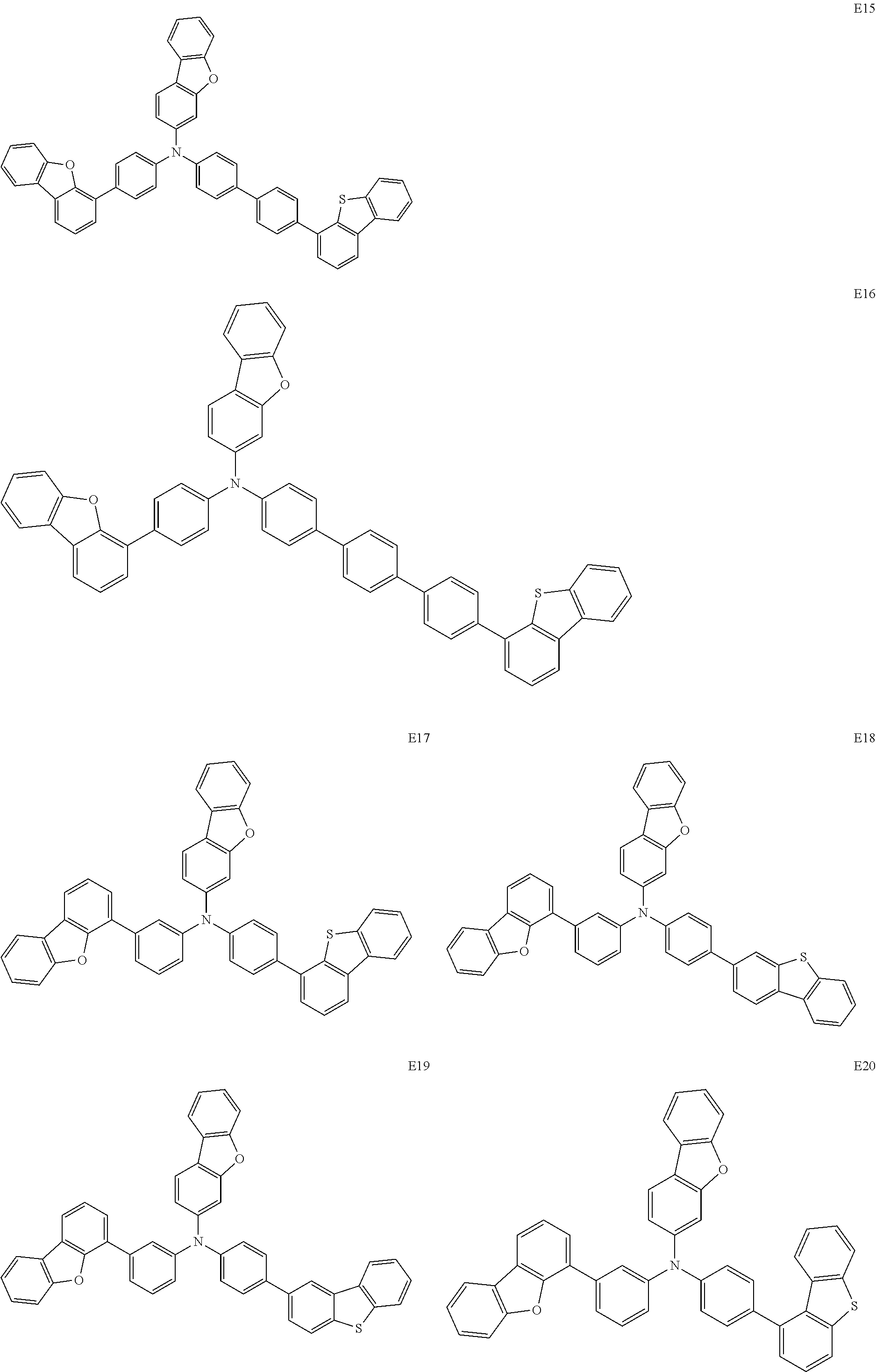 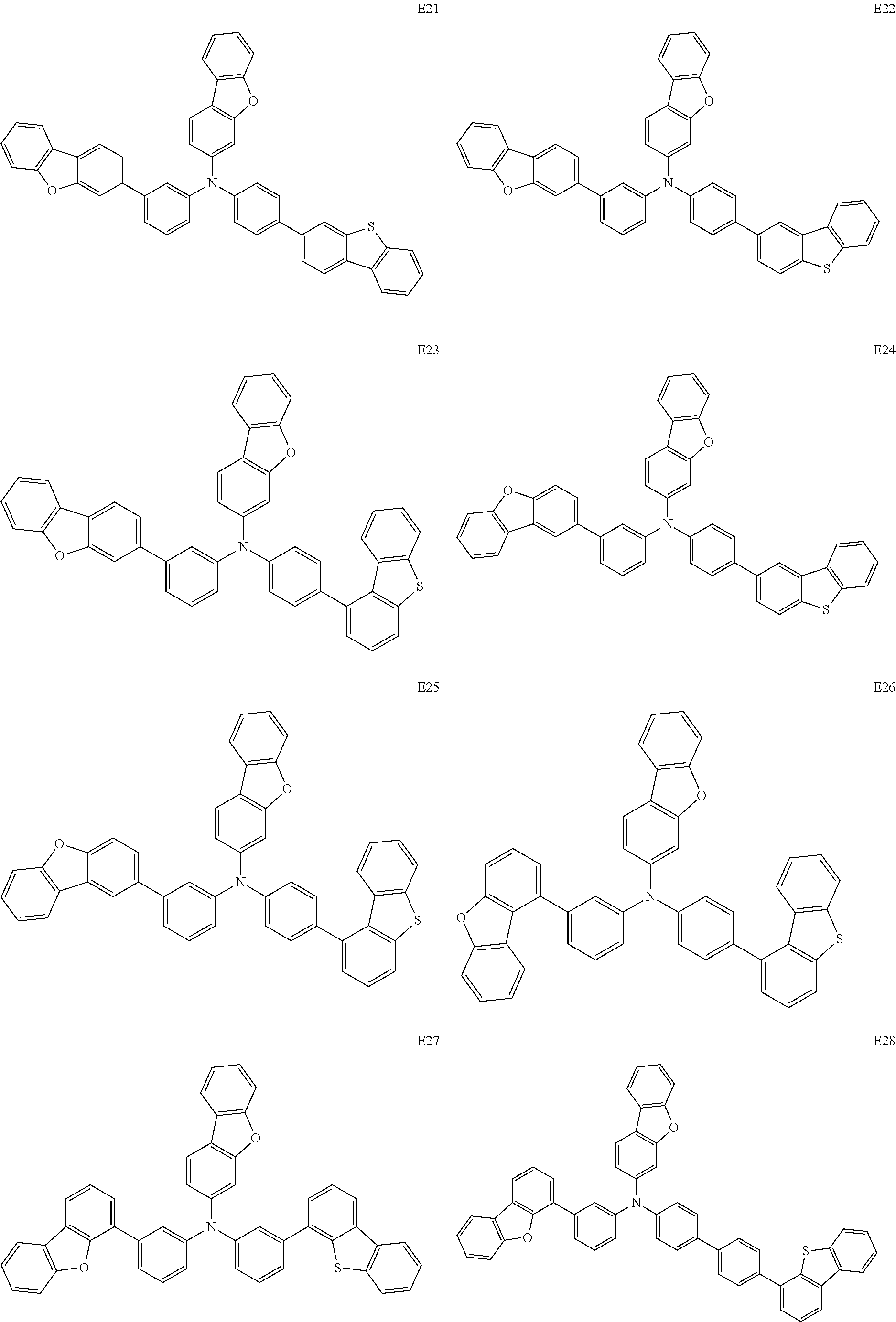 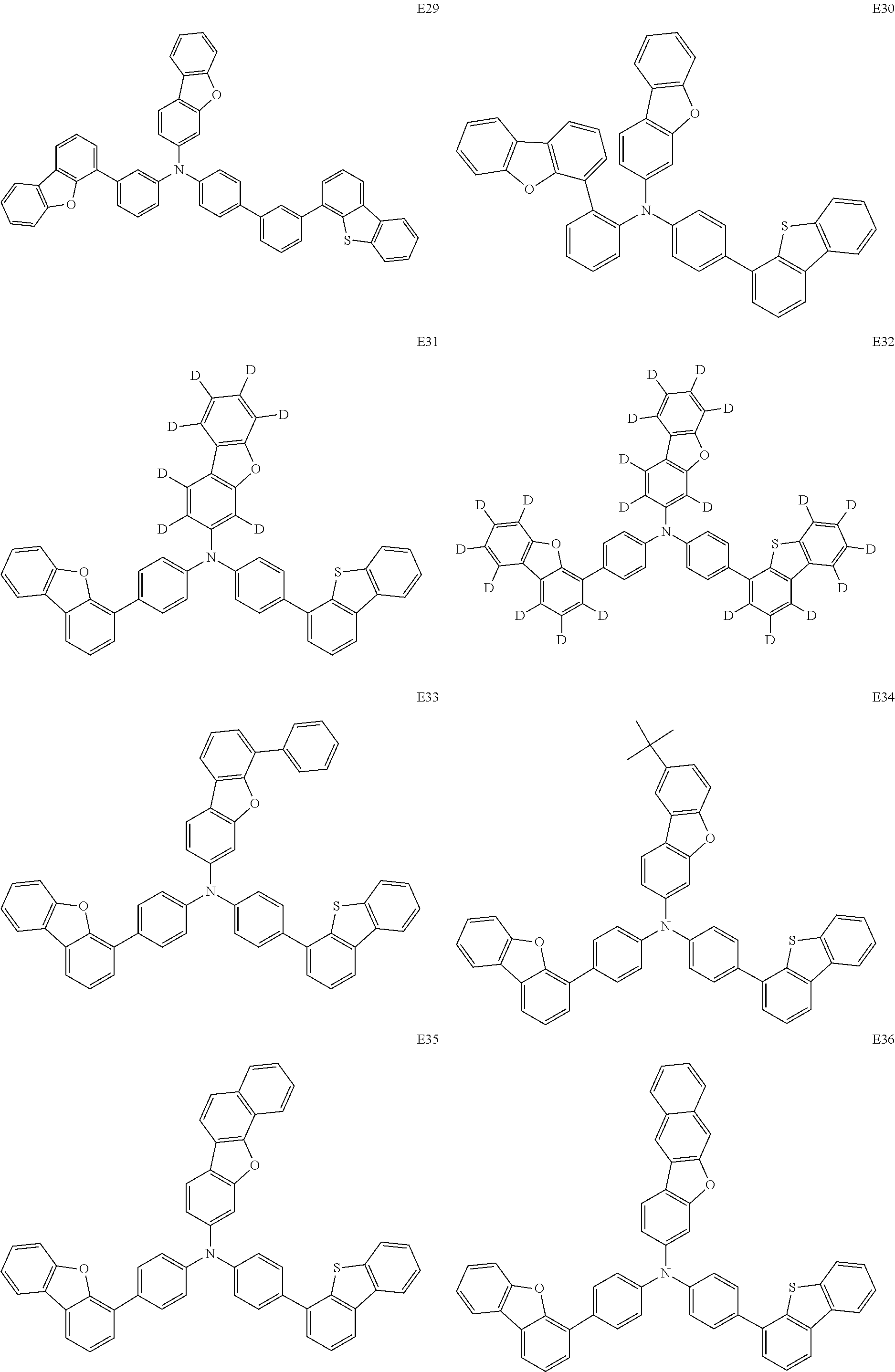 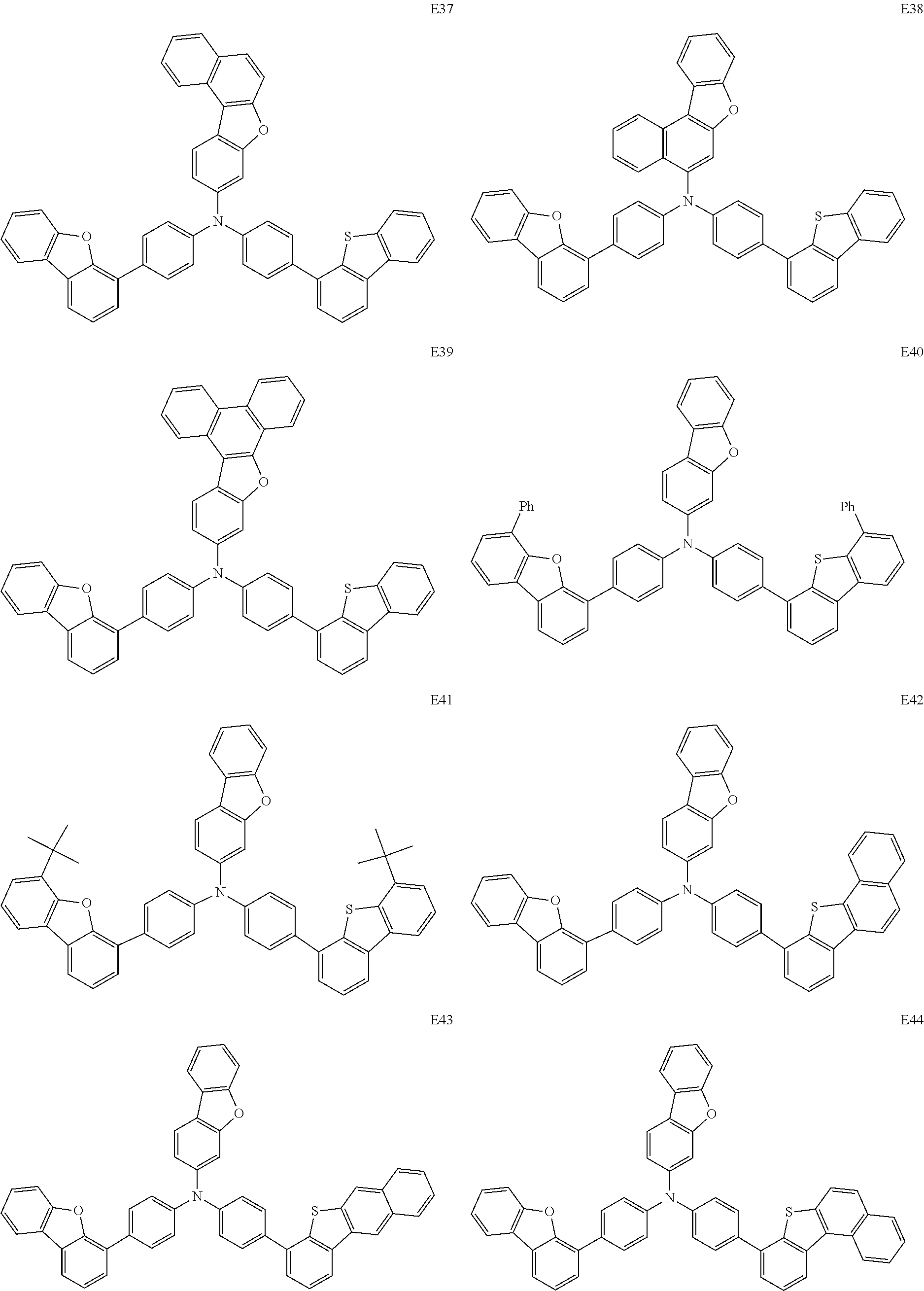 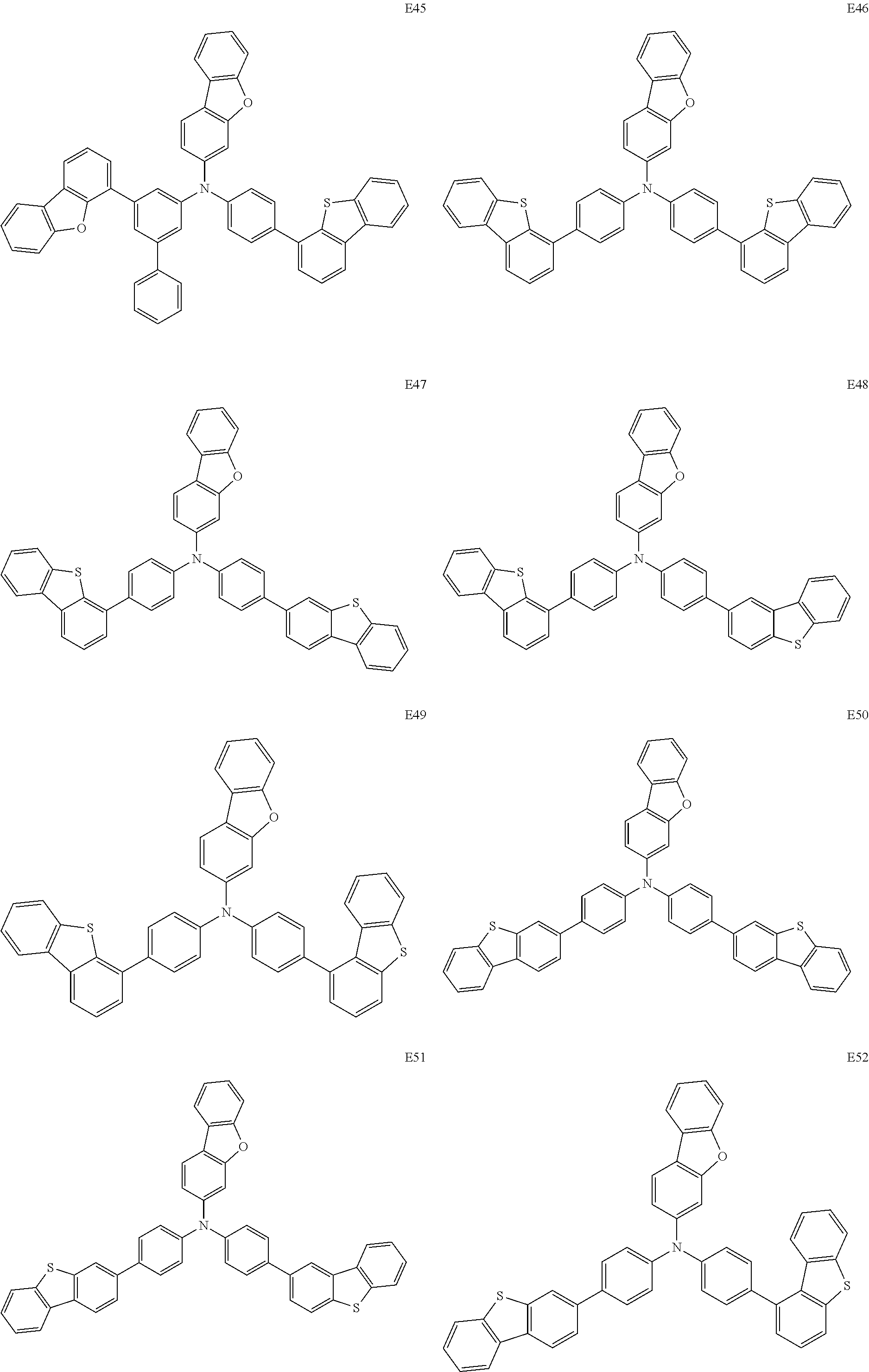 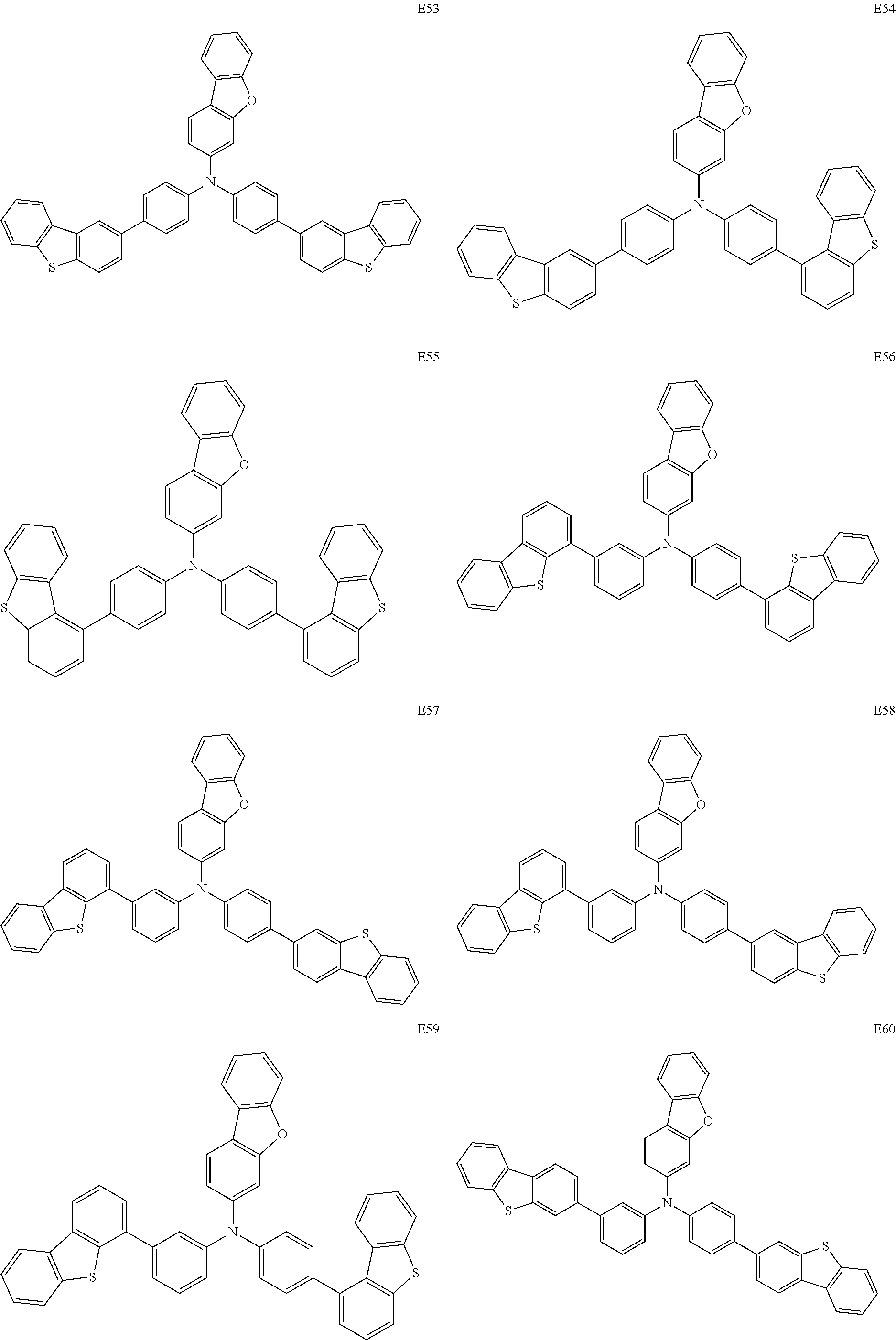 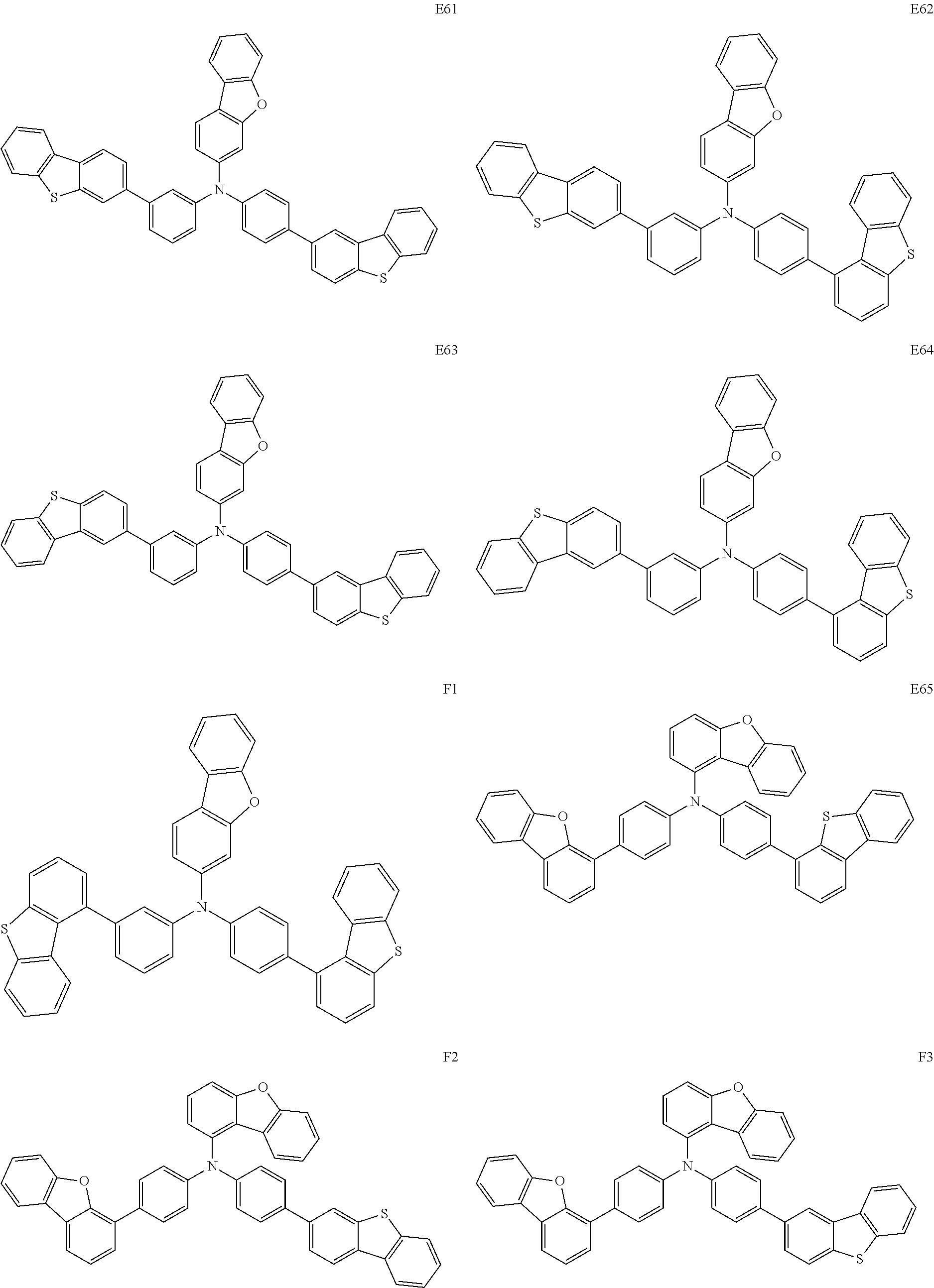 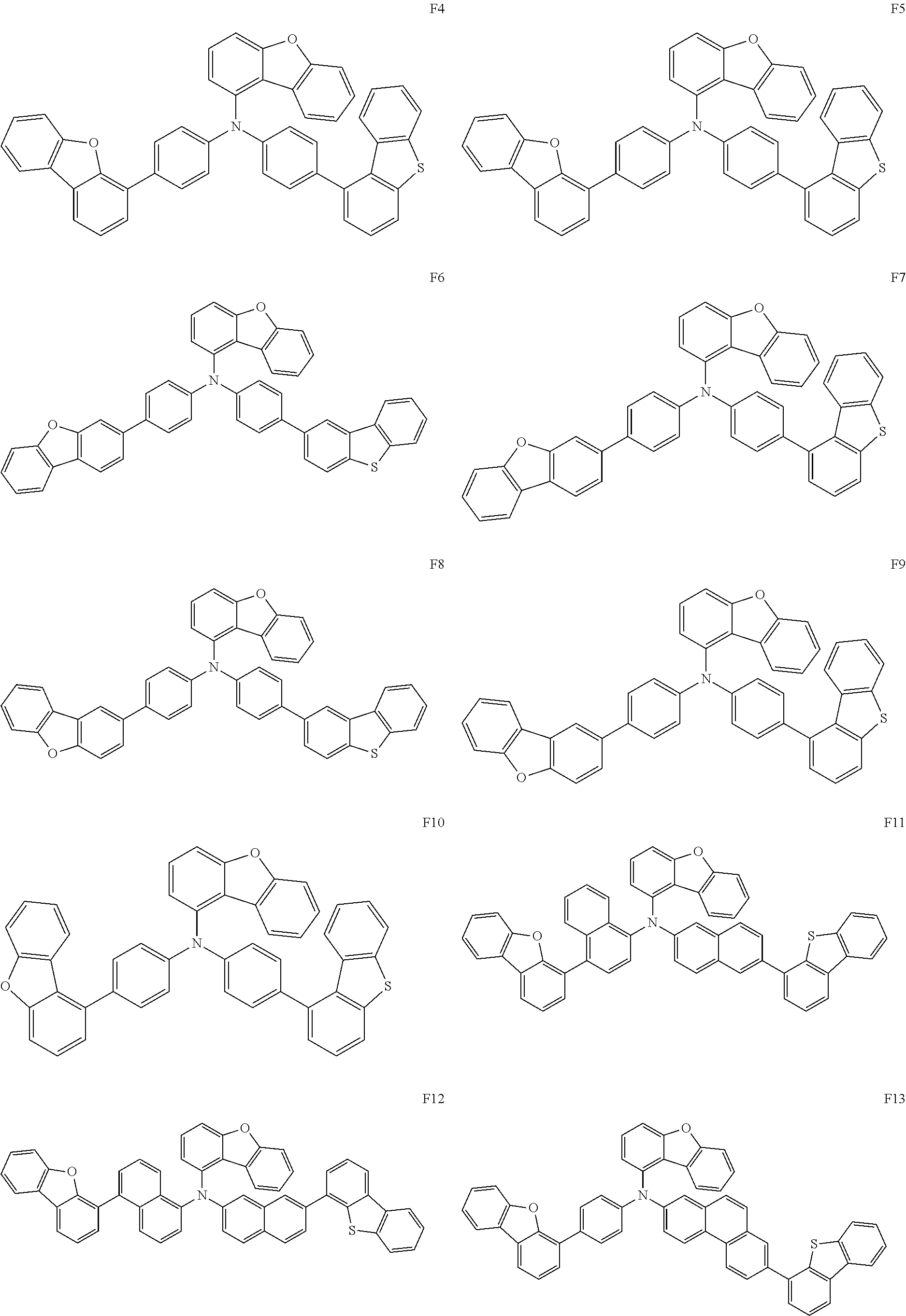 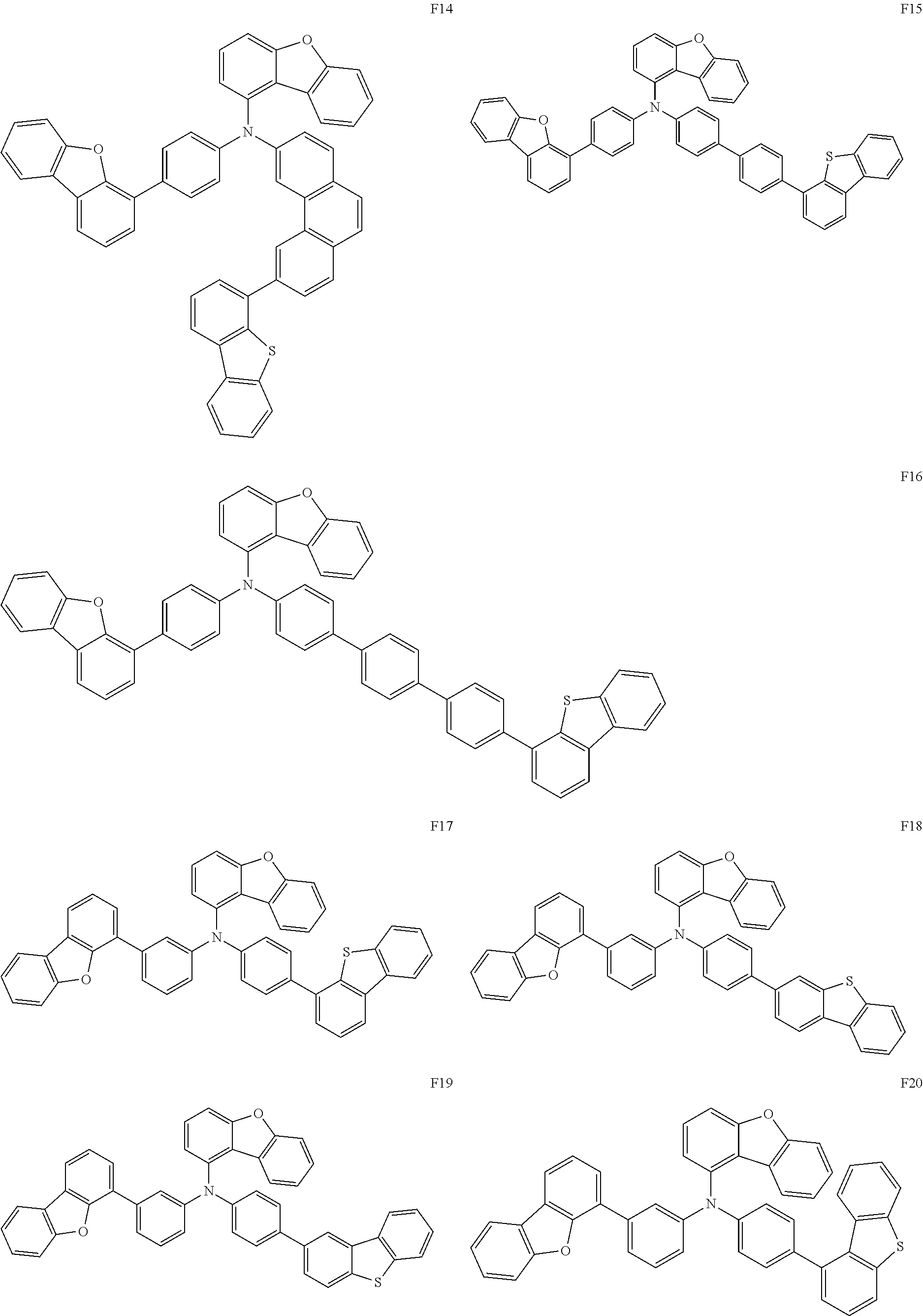 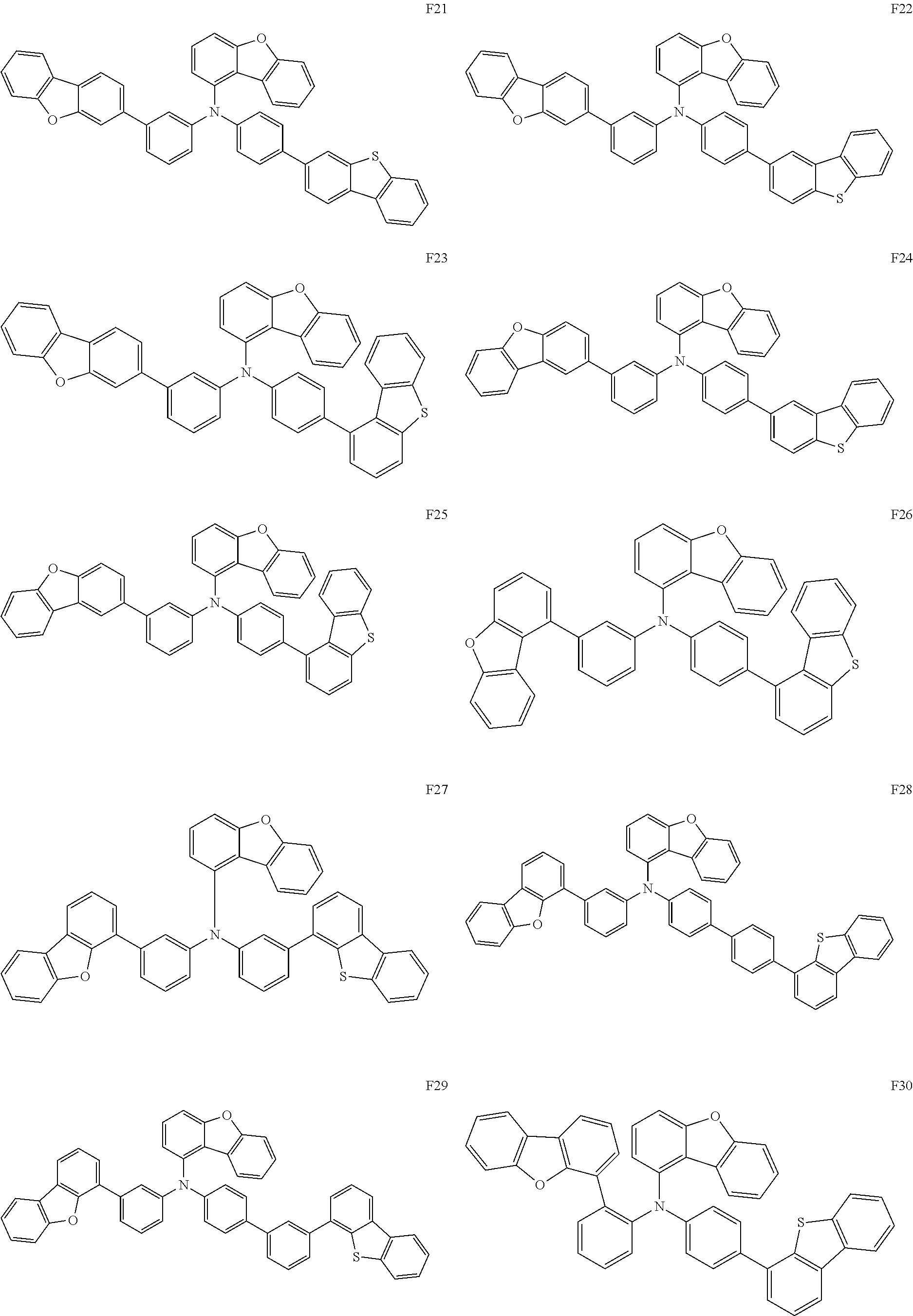 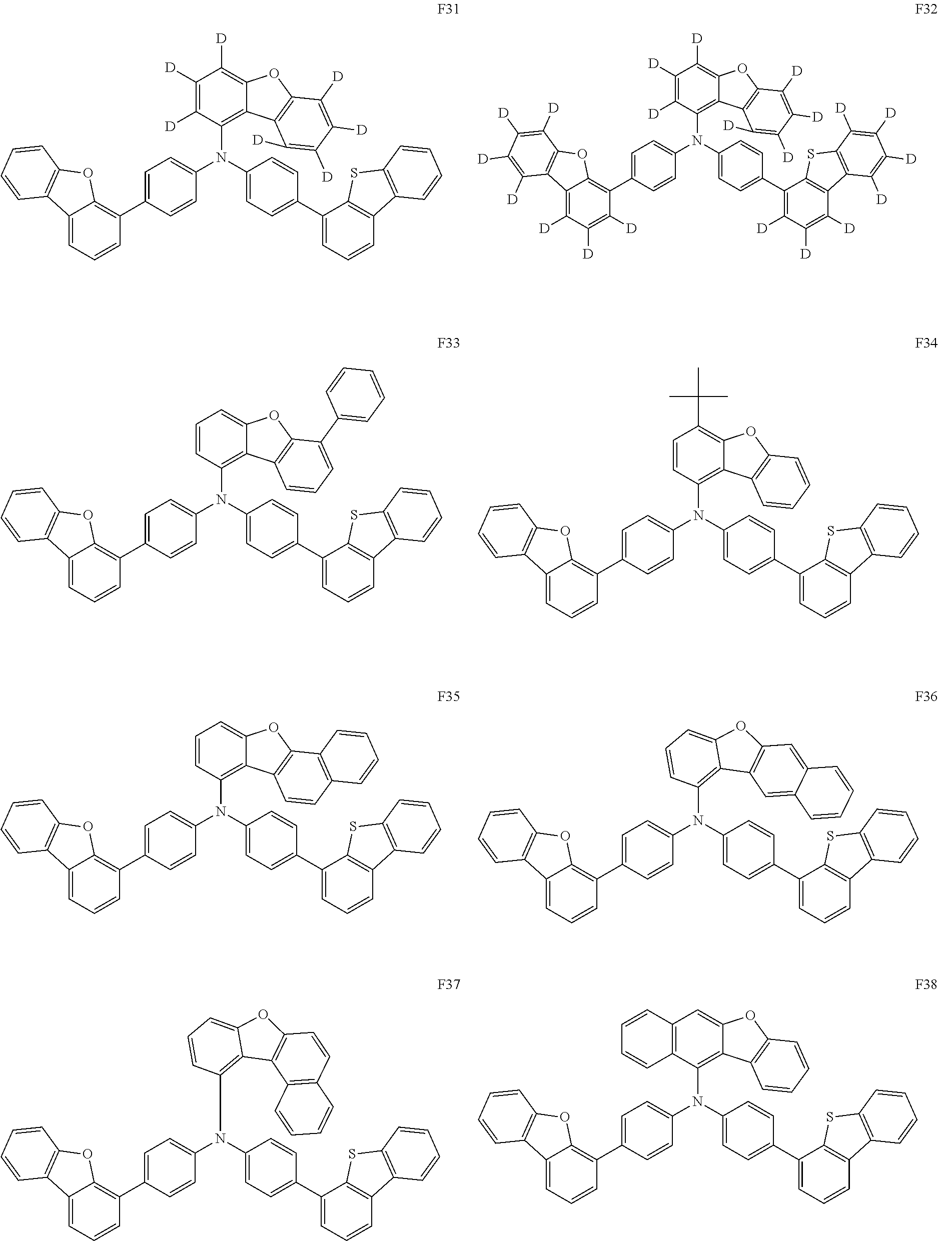 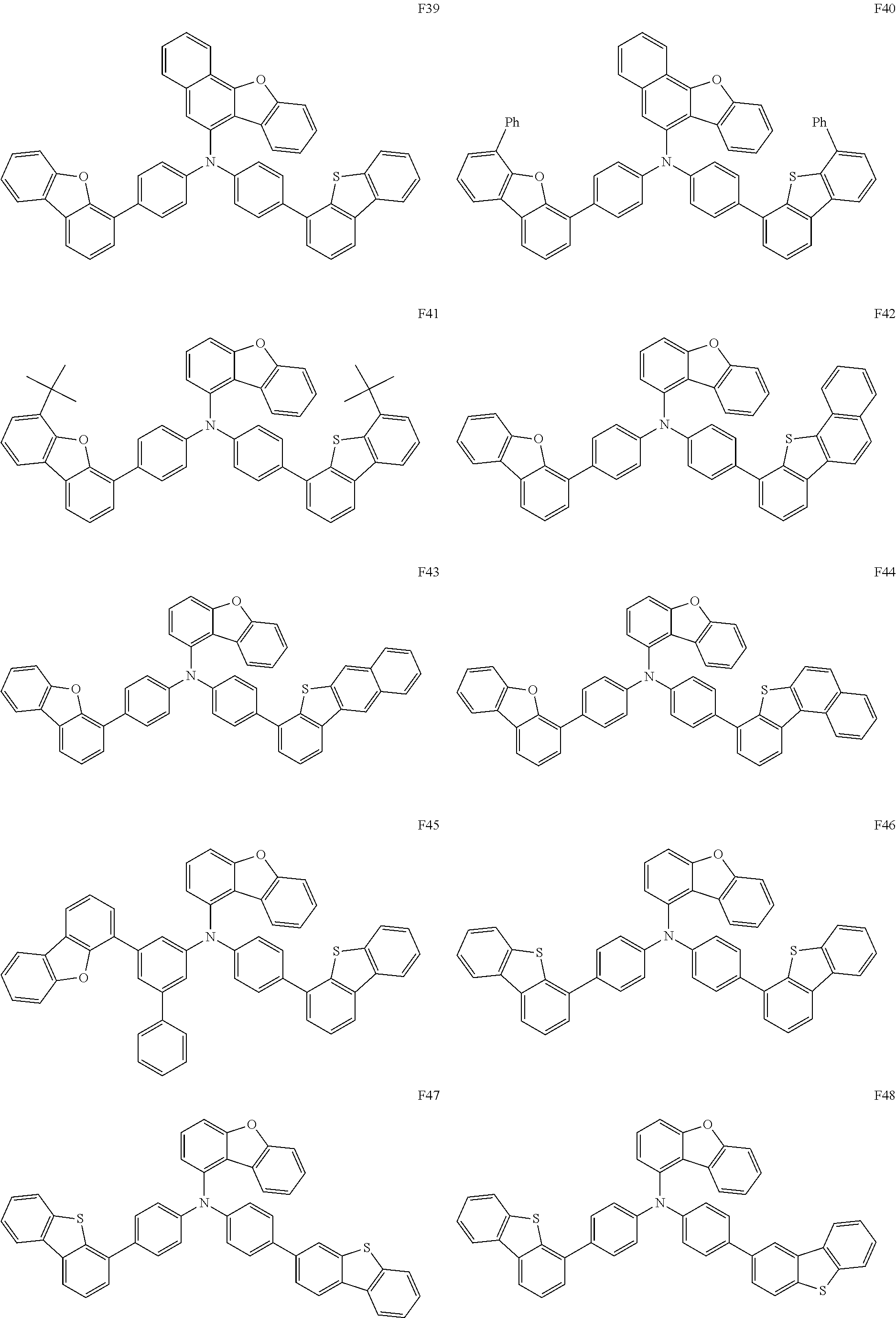 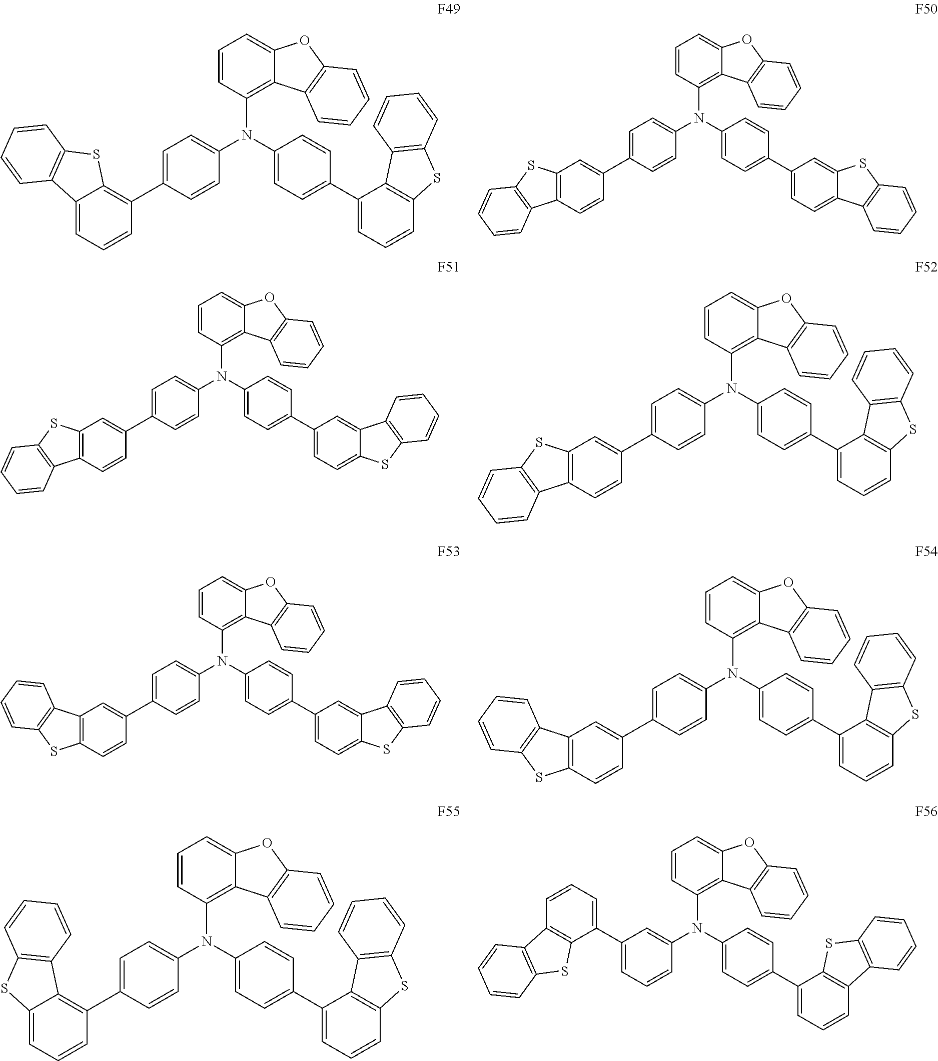 [chemical structure]
